(19) United States
(12) Patent Application Publication
(10) Pub. No.: US 2025/0041455 A1
(43) Pub. Date: Feb. 6, 2025

(54) ANTI-TFR:GAA AND ANTI-CD63:GAA INSERTION FOR TREATMENT OF POMPE DISEASE

(71) Applicant: Regeneron Pharmaceuticals, Inc., Tarrytown, NY (US)

(72) Inventors: Andrew Baik, Scarsdale, NY (US); Allen Lin, Woodside, NY (US); Maria Praggastis, Cortlandt Manor, NY (US); Katherine Cygnar, New York, NY (US); Evangelos Pefanis, Somers, NY (US); Leah Sabin, Goldens Bridge, NY (US)

(21) Appl. No.: 18/785,712

(22) Filed: Jul. 26, 2024

Related U.S. Application Data
(60) Provisional application No. 63/516,395, filed on Jul. 28, 2023.

(51) Int. Cl. A61K 48/00; A61K 38/47; A61K 47/68; A61P 3/00; C07K 16/28; C12N 9/22; C12N 15/11; C12N 15/86

(52) U.S. Cl. CPC A61K 48/0058; A61K 38/47; A61K 47/6815; A61K 48/0083; A61P 3/00; C07K 16/2881; C07K 16/2896; C12N 9/22; C12N 15/11; C12N 15/86; C12Y 302/0102; C07K 2317/565; C07K 2317/622; C07K 2317/92; C12N 2310/20; C12N 2750/14143; C12N 2800/80; C12N 2830/50

(57) ABSTRACT

Nucleic acid constructs and compositions that allow insertion of a multidomain therapeutic protein (e.g., GAA fusion protein) coding sequence into a target genomic locus such as an endogenous ALB locus and/or expression of the multidomain therapeutic protein (e.g., GAA fusion protein) coding sequence are also provided. The nucleic acid constructs and compositions can be used in methods of integration of a multidomain therapeutic protein (e.g., GAA fusion protein) nucleic acid into a target genomic locus, methods of expression of a multidomain therapeutic protein (e.g., GAA fusion protein) in a cell, methods of reducing glycogen accumulation, methods of treating Pompe disease or GAA deficiency in a subject, and method of preventing or reducing the onset of a sign or symptom of Pompe disease in a subject, including neonatal cells and subjects.

### CROSS-REFERENCE TO RELATED APPLICATIONS

This application claims the benefit of U.S. Application No. 63/516,395, filed Jul. 28, 2023, which is herein incorporated by reference in its entirety for all purposes.

### REFERENCE TO A SEQUENCE LISTING SUBMITTED AS AN XML FILE

The Sequence Listing written in file 616966SEQLIST.xml is 1,213,989 bytes, was created on Jul. 24, 2024, and is hereby incorporated by reference.

### BACKGROUND

Pompe disease (PD), or glycogen storage disease type II, is a monogenic, lysosomal disease caused by a deficiency in the activity of the enzyme lysosomal acid alpha-glucosidase (GAA). GAA deficiency results in an accumulation of its substrate, glycogen, in the lysosomes of cells in tissues including skeletal and cardiac muscle. This aberrant accumulation of glycogen in myofibers results in progressive damage of muscle tissue, with symptoms that can include cardiomegaly, mild to profound muscle weakness, and ultimately death due to cardiac or respiratory failure. Infantile onset PD (IOPD) is associated with GAA activity of <1% of normal. It is severe and affects visceral organs, muscles, and the central nervous system (CNS). Late onset PD (LOPD) is associated with GAA activity of 2-40%. It is less severe, with primarily respiratory and skeletal muscle involvement.

The only approved therapy for PD is enzyme replacement therapy (ERT). Recombinant human (rh) GAA is delivered by intravenous infusion into patients every other week. While ERT has been very successful in treating the cardiac manifestations of PD, skeletal muscle and the CNS remain minimally treated by ERT. The primary mechanism by which rhGAA reaches lysosomes is through uptake by the cation-independent mannose 6-phosphate (M6P) receptor (CIMPR), which binds M6P on rhGAA. However, CI-MPR expression in skeletal muscle is very low, and rhGAA is poorly mannose 6-phosphorylated. In addition, CI-MPR may be misdirected into autophagosomes in affected cells, rather than lysosomes, while a large amount of the drug is also taken up by the liver, an organ that does not have primary pathology in PD. The ERT does not cross the blood-brain-barrier. In addition, PD can require treatment early in life, which presents additional hurdles due to the unique environment in neonatal and juvenile patients.

### SUMMARY

Nucleic acid constructs and compositions that allow insertion of a multidomain therapeutic protein (e.g., GAA fusion protein) coding sequence into a target genomic locus such as an endogenous ALB locus and/or expression of the multidomain therapeutic protein (e.g., GAA fusion protein) coding sequence are provided. The nucleic acid constructs and compositions can be used in methods of integrating or inserting a multidomain therapeutic protein (e.g., GAA fusion protein) nucleic acid into a target genomic locus in a cell or a population of cells or a subject, methods of expressing a multidomain therapeutic protein (e.g., GAA fusion protein) in a cell or a population of cells or a subject, methods of reducing glycogen accumulation in a cell or a population of cells or a subject, and methods of treating Pompe disease or GAA deficiency in a subject, and method of preventing or reducing the onset of a sign or symptom of Pompe disease in a subject such as subjects with reduced GAA activity or expression and in a subject diagnosed with Pompe disease, including neonatal subjects. In some embodiments the cell, population of cells, or subject is a neonatal cell, a neonatal population of cells, or a neonatal subject.

In one aspect, provided are compositions comprising a nucleic acid construct comprising a coding sequence for a multidomain therapeutic protein comprising a delivery domain fused to a lysosomal alpha-glucosidase polypeptide, wherein the lysosomal alpha-glucosidase coding sequence is CpG-depleted relative to a wild type lysosomal alpha-glucosidase coding sequence, optionally wherein the delivery domain is a CD63-binding delivery domain or a TfR-binding delivery domain. In some such compositions, the nucleic acid construct comprises a polyadenylation signal or sequence downstream of the coding sequence for the multidomain therapeutic protein. In some such compositions, the polyadenylation signal comprises a bovine growth hormone (BGH) polyadenylation signal, a simian virus 40 (SV40) polyadenylation signal, or a combination of the bovine growth hormone polyadenylation signal and the SV40 polyadenylation signal. In some such compositions, the SV40 polyadenylation signal is a unidirectional SV40 late polyadenylation signal, wherein each instance of the sequence AATAAA in the reverse strand is mutated in the unidirectional SV40 late polyadenylation signal, optionally wherein the SV40 polyadenylation signal is at least 95%, at least 96%, at least 97%, at least 98%, or at least 99% identical to the sequence set forth in SEQ ID NO: 752, and optionally wherein the SV40 polyadenylation signal comprises the sequence set forth in SEQ ID NO: 752. In some such compositions, the polyadenylation signal comprises the BGH polyadenylation signal, optionally wherein the BGH polyadenylation signal is at least 95%, at least 96%, at least 97%, at least 98%, or at least 99% identical to the sequence set forth in SEQ ID NO: 751, optionally wherein the BGH polyadenylation signal comprises the sequence set forth in SEQ ID NO: 751. In some such compositions, the polyadenylation signal comprises the BGH polyadenylation signal and the SV40 polyadenylation signal, optionally wherein the BGH polyadenylation signal comprises the sequence set forth in SEQ ID NO: 751, and optionally wherein the SV40 polyadenylation signal comprises the sequence set forth in SEQ ID NO: 752, optionally wherein the polyadenylation signal comprising the BGH polyadenylation signal and the SV40 polyadenylation signal is at least 95%, at least 96%, at least 97%, at least 98%, or at least 99% identical to the sequence set forth in SEQ ID NO: 795, and optionally wherein the polyadenylation signal comprising the BGH polyadenylation signal and the SV40 polyadenylation signal comprises the sequence set forth in SEQ ID NO: 795. In some such compositions, the nucleic acid construct is a unidirectional nucleic acid construct.

In some such compositions, the coding sequence for the delivery domain is modified to remove one or more cryptic splice sites, the coding sequence for the lysosomal alpha-glucosidase polypeptide is modified to remove one or more cryptic splice sites, or the coding sequence for the multidomain therapeutic protein is modified to remove one or more cryptic splice sites. In some such compositions, the coding sequence for the delivery domain is CpG-depleted, or the coding sequence for the multidomain therapeutic protein is CpG-depleted. In some such compositions, the coding sequence for the delivery domain is codon-optimized and CpG-depleted, the coding sequence for the lysosomal alpha-glucosidase polypeptide is codon-optimized and CpG-depleted, or the coding sequence for the multidomain therapeutic protein is codon-optimized and CpG-depleted.

In some such compositions, the nucleic acid construct comprises a splice acceptor upstream of the coding sequence for the multidomain therapeutic protein. In some such compositions, the nucleic acid construct does not comprise a homology arm. In some such compositions, the nucleic acid construct comprises from 5′ to 3′: a splice acceptor, the coding sequence for the multidomain therapeutic protein, and a polyadenylation signal or sequence, wherein the nucleic acid construct does not comprise a promoter that drives the expression of the multidomain therapeutic protein, and wherein the nucleic acid construct does not comprise a homology arm. In some such compositions, the nucleic acid construct comprises homology arms. In some such compositions, the nucleic acid construct does not comprise a promoter that drives the expression of the multidomain therapeutic protein. In some such compositions, the coding sequence for the multidomain therapeutic protein is operably linked to a promoter, optionally wherein the promoter is a liver-specific promoter.

In some such compositions, the C-terminus of the delivery domain is fused to the N-terminus of the lysosomal alpha-glucosidase polypeptide. In some such compositions, the delivery domain is fused to the lysosomal alpha-glucosidase polypeptide via a peptide linker. In some such compositions, the lysosomal alpha-glucosidase polypeptide lacks the lysosomal alpha-glucosidase signal peptide and propeptide. In some such compositions, the lysosomal alpha-glucosidase polypeptide comprises, consists essentially of, or consists of the sequence set forth in SEQ ID NO: 727. In some such compositions, the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 750, optionally wherein the nucleotide at position 1095 is a G, the nucleotide at position 1098 is a C, and the nucleotide at position 2343 is a G. In some such compositions, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NO: 750 and encodes a lysosomal alpha-glucosidase protein comprising SEQ ID NO: 727, optionally wherein the nucleotide at position 1095 is a G, the nucleotide at position 1098 is a C, and the nucleotide at position 2343 is a G. In some such compositions, the lysosomal alpha-glucosidase coding sequence comprises, consists essentially of, or consists of the sequence set forth in any one of SEQ ID NO: 750. In some such compositions, the lysosomal alpha-glucosidase coding sequence is at least 90%, at least 91%, at least 92%, at least 93%, at least 94%, at least 95%, at least 96%, at least 97%, at least 98%, or at least 99% identical to SEQ ID NO: 749, optionally wherein the nucleotide at position 2343 is a G. In some such compositions, the lysosomal alpha-glucosidase coding sequence is at least 90%, at least 91%, at least 92%, at least 93%, at least 94%, at least 95%, at least 96%, at least 97%, at least 98%, or at least 99% identical to any one of SEQ ID NO: 749 and encodes a lysosomal alpha-glucosidase protein comprising SEQ ID NO: 727, optionally wherein the nucleotide at position 2343 is a G. In some such compositions, the lysosomal alpha-glucosidase coding sequence comprises, consists essentially of, or consists of the sequence set forth in any one of SEQ ID NO: 749.

In some such compositions, the delivery domain is the TfR-binding delivery domain. In some such compositions, the TfR-binding delivery domain comprises an anti-TfR antigen-binding protein, optionally wherein the antigen-binding protein binds to human transferrin receptor with a K_{D} of about 41 nM or a stronger affinity, optionally wherein the antigen-binding protein binds to human transferrin receptor with a K_{D} of about 3 nM or a stronger affinity, or optionally wherein the antigen-binding protein binds to human transferrin receptor with a K_{D} of about 0.45 nM to 3 nM. In some such compositions, the anti-TfR antigen binding protein comprises: (i) a HCVR that comprises the HCDR1, HCDR2 and HCDR3 of a HCVR comprising the amino acid sequence set forth in SEQ ID NO: 171, 181, 191, 201, 211, 221, 231, 241, 251, 261, 271, 281, 291, 301, 311, 321, 331, 341, 351, 361, 371, 381, 391, 401, 411, 421, 431, 441, 451, 461, 471, or 481 (or a variant thereof); and/or (ii) a LCVR that comprises the LCDR1, LCDR2 and LCDR3 of a LCVR comprising the amino acid sequence set forth in SEQ ID NO: 176, 186, 196, 206, 216, 226, 236, 246, 256, 266, 276, 286, 296, 306, 316, 326, 336, 346, 356, 366, 376, 386, 396, 406, 416, 426, 436, 446, 456, 466, 476, or 486 (or a variant thereof).

In some such compositions, the anti-TfR antigen binding protein comprises: (1) a HCVR comprising the HCDR1, HCDR2 and HCDR3 of a HCVR that comprises the amino acid sequence set forth in SEQ ID NO: 171 (or a variant thereof); and a LCVR comprising the LCDR1, LCDR2 and LCDR3 of a LCVR that comprises the amino acid sequence set forth in SEQ ID NO: 176 (or a variant thereof); (2) a HCVR comprising the HCDR1, HCDR2 and HCDR3 of a HCVR that comprises the amino acid sequence set forth in SEQ ID NO: 181 (or a variant thereof); and a LCVR comprising the LCDR1, LCDR2 and LCDR3 of a LCVR that comprises the amino acid sequence set forth in SEQ ID NO: 186 (or a variant thereof); (3) a HCVR comprising the HCDR1, HCDR2 and HCDR3 of a HCVR that comprises the amino acid sequence set forth in SEQ ID NO: 191 (or a variant thereof); and a LCVR comprising the LCDR1, LCDR2 and LCDR3 of a LCVR that comprises the amino acid sequence set forth in SEQ ID NO: 196 (or a variant thereof); (4) a HCVR comprising the HCDR1, HCDR2 and HCDR3 of a HCVR that comprises the amino acid sequence set forth in SEQ ID NO: 201 (or a variant thereof); and a LCVR comprising the LCDR1, LCDR2 and LCDR3 of a LCVR that comprises the amino acid sequence set forth in SEQ ID NO: 206 (or a variant thereof); (5) a HCVR comprising the HCDR1, HCDR2 and HCDR3 of a HCVR that comprises the amino acid sequence set forth in SEQ ID NO: 211 (or a variant thereof); and a LCVR comprising the LCDR1, LCDR2 and LCDR3 of a LCVR that comprises the amino acid sequence set forth in SEQ ID NO: 216 (or a variant thereof); (6) a HCVR comprising the HCDR1, HCDR2 and HCDR3 of a HCVR that comprises the amino acid sequence set forth in SEQ ID NO: 221 (or a variant thereof); and a LCVR comprising the LCDR1, LCDR2 and LCDR3 of a LCVR that comprises the amino acid sequence set forth in SEQ ID NO: 226 (or a variant thereof); (7) a HCVR comprising the HCDR1, HCDR2 and HCDR3 of a HCVR that comprises the amino acid sequence set forth in SEQ ID NO: 231 (or a variant thereof); and a LCVR comprising the LCDR1, LCDR2 and LCDR3 of a LCVR that comprises the amino acid sequence set forth in SEQ ID NO: 236 (or a variant thereof); (8) a HCVR comprising the HCDR1, HCDR2 and HCDR3 of a HCVR that comprises the amino acid sequence set forth in SEQ ID NO: 241 (or a variant thereof); and a LCVR comprising the LCDR1, LCDR2 and LCDR3 of a LCVR that comprises the amino acid sequence set forth in SEQ ID NO: 246 (or a variant thereof); (9) a HCVR comprising the HCDR1, HCDR2 and HCDR3 of a HCVR that comprises the amino acid sequence set forth in SEQ ID NO: 251 (or a variant thereof); and a LCVR comprising the LCDR1, LCDR2 and LCDR3 of a LCVR that comprises the amino acid sequence set forth in SEQ ID NO: 256 (or a variant thereof); (10) a HCVR comprising the HCDR1, HCDR2 and HCDR3 of a HCVR that comprises the amino acid sequence set forth in SEQ ID NO: 261 (or a variant thereof); and a LCVR comprising the LCDR1, LCDR2 and LCDR3 of a LCVR that comprises the amino acid sequence set forth in SEQ ID NO: 266 (or a variant thereof); (11) a HCVR comprising the HCDR1, HCDR2 and HCDR3 of a HCVR that comprises the amino acid sequence set forth in SEQ ID NO: 271 (or a variant thereof); and a LCVR comprising the LCDR1, LCDR2 and LCDR3 of a LCVR that comprises the amino acid sequence set forth in SEQ ID NO: 276 (or a variant thereof); (12) a HCVR comprising the HCDR1, HCDR2 and HCDR3 of a HCVR that comprises the amino acid sequence set forth in SEQ ID NO: 281 (or a variant thereof); and a LCVR comprising the LCDR1, LCDR2 and LCDR3 of a LCVR that comprises the amino acid sequence set forth in SEQ ID NO: 286 (or a variant thereof); (13) a HCVR comprising the HCDR1, HCDR2 and HCDR3 of a HCVR that comprises the amino acid sequence set forth in SEQ ID NO: 291 (or a variant thereof); and a LCVR comprising the LCDR1, LCDR2 and LCDR3 of a LCVR that comprises the amino acid sequence set forth in SEQ ID NO: 296 (or a variant thereof); (14) a HCVR comprising the HCDR1, HCDR2 and HCDR3 of a HCVR that comprises the amino acid sequence set forth in SEQ ID NO: 301 (or a variant thereof); and a LCVR comprising the LCDR1, LCDR2 and LCDR3 of a LCVR that comprises the amino acid sequence set forth in SEQ ID NO: 306 (or a variant thereof); (15) a HCVR comprising the HCDR1, HCDR2 and HCDR3 of a HCVR that comprises the amino acid sequence set forth in SEQ ID NO: 311 (or a variant thereof); and a LCVR comprising the LCDR1, LCDR2 and LCDR3 of a LCVR that comprises the amino acid sequence set forth in SEQ ID NO: 316 (or a variant thereof); (16) a HCVR comprising the HCDR1, HCDR2 and HCDR3 of a HCVR that comprises the amino acid sequence set forth in SEQ ID NO: 321 (or a variant thereof); and a LCVR comprising the LCDR1, LCDR2 and LCDR3 of a LCVR that comprises the amino acid sequence set forth in SEQ ID NO: 326 (or a variant thereof); (17) a HCVR comprising the HCDR1, HCDR2 and HCDR3 of a HCVR that comprises the amino acid sequence set forth in SEQ ID NO: 331 (or a variant thereof); and a LCVR comprising the LCDR1, LCDR2 and LCDR3 of a LCVR that comprises the amino acid sequence set forth in SEQ ID NO: 336 (or a variant thereof); (18) a HCVR comprising the HCDR1, HCDR2 and HCDR3 of a HCVR that comprises the amino acid sequence set forth in SEQ ID NO: 341 (or a variant thereof); and a LCVR comprising the LCDR1, LCDR2 and LCDR3 of a LCVR that comprises the amino acid sequence set forth in SEQ ID NO: 346 (or a variant thereof); (19) a HCVR comprising the HCDR1, HCDR2 and HCDR3 of a HCVR that comprises the amino acid sequence set forth in SEQ ID NO: 351 (or a variant thereof); and a LCVR comprising the LCDR1, LCDR2 and LCDR3 of a LCVR that comprises the amino acid sequence set forth in SEQ ID NO: 356 (or a variant thereof); (20) a HCVR comprising the HCDR1, HCDR2 and HCDR3 of a HCVR that comprises the amino acid sequence set forth in SEQ ID NO: 361 (or a variant thereof); and a LCVR comprising the LCDR1, LCDR2 and LCDR3 of a LCVR that comprises the amino acid sequence set forth in SEQ ID NO: 366 (or a variant thereof); (21) a HCVR comprising the HCDR1, HCDR2 and HCDR3 of a HCVR that comprises the amino acid sequence set forth in SEQ ID NO: 371 (or a variant thereof); and a LCVR comprising the LCDR1, LCDR2 and LCDR3 of a LCVR that comprises the amino acid sequence set forth in SEQ ID NO: 376 (or a variant thereof); (22) a HCVR comprising the HCDR1, HCDR2 and HCDR3 of a HCVR that comprises the amino acid sequence set forth in SEQ ID NO: 381 (or a variant thereof); and a LCVR comprising the LCDR1, LCDR2 and LCDR3 of a LCVR that comprises the amino acid sequence set forth in SEQ ID NO: 386 (or a variant thereof); (23) a HCVR comprising the HCDR1, HCDR2 and HCDR3 of a HCVR that comprises the amino acid sequence set forth in SEQ ID NO: 391 (or a variant thereof); and a LCVR comprising the LCDR1, LCDR2 and LCDR3 of a LCVR that comprises the amino acid sequence set forth in SEQ ID NO: 396 (or a variant thereof); (24) a HCVR comprising the HCDR1, HCDR2 and HCDR3 of a HCVR that comprises the amino acid sequence set forth in SEQ ID NO: 401 (or a variant thereof); and a LCVR comprising the LCDR1, LCDR2 and LCDR3 of a LCVR that comprises the amino acid sequence set forth in SEQ ID NO: 406 (or a variant thereof); (25) a HCVR comprising the HCDR1, HCDR2 and HCDR3 of a HCVR that comprises the amino acid sequence set forth in SEQ ID NO: 411 (or a variant thereof); and a LCVR comprising the LCDR1, LCDR2 and LCDR3 of a LCVR that comprises the amino acid sequence set forth in SEQ ID NO: 416 (or a variant thereof); (26) a HCVR comprising the HCDR1, HCDR2 and HCDR3 of a HCVR that comprises the amino acid sequence set forth in SEQ ID NO: 421 (or a variant thereof); and a LCVR comprising the LCDR1, LCDR2 and LCDR3 of a LCVR that comprises the amino acid sequence set forth in SEQ ID NO: 426 (or a variant thereof); (27) a HCVR comprising the HCDR1, HCDR2 and HCDR3 of a HCVR that comprises the amino acid sequence set forth in SEQ ID NO: 431 (or a variant thereof); and a LCVR comprising the LCDR1, LCDR2 and LCDR3 of a LCVR that comprises the amino acid sequence set forth in SEQ ID NO: 436 (or a variant thereof); (28) a HCVR comprising the HCDR1, HCDR2 and HCDR3 of a HCVR that comprises the amino acid sequence set forth in SEQ ID NO: 441 (or a variant thereof); and a LCVR comprising the LCDR1, LCDR2 and LCDR3 of a LCVR that comprises the amino acid sequence set forth in SEQ ID NO: 446 (or a variant thereof); (29) a HCVR comprising the HCDR1, HCDR2 and HCDR3 of a HCVR that comprises the amino acid sequence set forth in SEQ ID NO: 451 (or a variant thereof); and a LCVR comprising the LCDR1, LCDR2 and LCDR3 of a LCVR that comprises the amino acid sequence set forth in SEQ ID NO: 456 (or a variant thereof); (30) a HCVR comprising the HCDR1, HCDR2 and HCDR3 of a HCVR that comprises the amino acid sequence set forth in SEQ ID NO: 461 (or a variant thereof); and a LCVR comprising the LCDR1, LCDR2 and LCDR3 of a LCVR that comprises the amino acid sequence set forth in SEQ ID NO: 466 (or a variant thereof); (31) a HCVR comprising the HCDR1, HCDR2 and HCDR3 of a HCVR that comprises the amino acid sequence set forth in SEQ ID NO: 471 (or a variant thereof); and a LCVR comprising the LCDR1, LCDR2 and LCDR3 of a LCVR that comprises the amino acid sequence set forth in SEQ ID NO: 476 (or a variant thereof); or (32) a HCVR comprising the HCDR1, HCDR2 and HCDR3 of a HCVR that comprises the amino acid sequence set forth in SEQ ID NO: 481 (or a variant thereof); and a LCVR comprising the LCDR1, LCDR2 and LCDR3 of a LCVR that comprises the amino acid sequence set forth in SEQ ID NO: 486 (or a variant thereof).

In some such compositions, the anti-TfR antigen binding protein comprises: (1) a HCVR comprising the HCDR1, HCDR2 and HCDR3 of a HCVR that comprises the amino acid sequence set forth in SEQ ID NO: 391 (or a variant thereof); and a LCVR comprising the LCDR1, LCDR2 and LCDR3 of a LCVR that comprises the amino acid sequence set forth in SEQ ID NO: 396 (or a variant thereof); or (2) a HCVR comprising the HCDR1, HCDR2 and HCDR3 of a HCVR that comprises the amino acid sequence set forth in SEQ ID NO: 411 (or a variant thereof); and a LCVR comprising the LCDR1, LCDR2 and LCDR3 of a LCVR that comprises the amino acid sequence set forth in SEQ ID NO: 416 (or a variant thereof). In some such compositions, the anti-TfR antigen binding protein comprises: a HCVR comprising the HCDR1, HCDR2 and HCDR3 of a HCVR that comprises the amino acid sequence set forth in SEQ ID NO: 391 (or a variant thereof); and a LCVR comprising the LCDR1, LCDR2 and LCDR3 of a LCVR that comprises the amino acid sequence set forth in SEQ ID NO: 396 (or a variant thereof).

In some such compositions, the anti-TfR antigen binding protein comprises: (a) a HCVR that comprises: an HCDR1 comprising the amino acid sequence set forth in SEQ ID NO: 172 (or a variant thereof), an HCDR2 comprising the amino acid sequence set forth in SEQ ID NO: 173 (or a variant thereof), and an HCDR3 comprising the amino acid sequence set forth in SEQ ID NO: 174 (or a variant thereof); and a LCVR that comprises: an LCDR1 comprising the amino acid sequence set forth in SEQ ID NO: 177 (or a variant thereof), an LCDR2 comprising the amino acid sequence set forth in SEQ ID NO: 178 (or a variant thereof), and an LCDR3 comprising the amino acid sequence set forth in SEQ ID NO: 179 (or a variant thereof); (b) a HCVR that comprises: an HCDR1 comprising the amino acid sequence set forth in SEQ ID NO: 182 (or a variant thereof), an HCDR2 comprising the amino acid sequence set forth in SEQ ID NO: 183 (or a variant thereof), and an HCDR3 comprising the amino acid sequence set forth in SEQ ID NO: 184 (or a variant thereof); and a LCVR that comprises: an LCDR1 comprising the amino acid sequence set forth in SEQ ID NO: 187 (or a variant thereof), an LCDR2 comprising the amino acid sequence set forth in SEQ ID NO: 188 (or a variant thereof), and an LCDR3 comprising the amino acid sequence set forth in SEQ ID NO: 189 (or a variant thereof); (c) a HCVR that comprises: an HCDR1 comprising the amino acid sequence set forth in SEQ ID NO: 192 (or a variant thereof), an HCDR2 comprising the amino acid sequence set forth in SEQ ID NO: 193 (or a variant thereof), and an HCDR3 comprising the amino acid sequence set forth in SEQ ID NO: 194 (or a variant thereof); and a LCVR that comprises: an LCDR1 comprising the amino acid sequence set forth in SEQ ID NO: 197 (or a variant thereof), an LCDR2 comprising the amino acid sequence set forth in SEQ ID NO: 198 (or a variant thereof), and an LCDR3 comprising the amino acid sequence set forth in SEQ ID NO: 199 (or a variant thereof); (d) a HCVR that comprises: an HCDR1 comprising the amino acid sequence set forth in SEQ ID NO: 202 (or a variant thereof), an HCDR2 comprising the amino acid sequence set forth in SEQ ID NO: 203 (or a variant thereof), and an HCDR3 comprising the amino acid sequence set forth in SEQ ID NO: 204 (or a variant thereof); and a LCVR that comprises: an LCDR1 comprising the amino acid sequence set forth in SEQ ID NO: 207 (or a variant thereof), an LCDR2 comprising the amino acid sequence set forth in SEQ ID NO: 208 (or a variant thereof), and an LCDR3 comprising the amino acid sequence set forth in SEQ ID NO: 209 (or a variant thereof); (e) a HCVR that comprises: an HCDR1 comprising the amino acid sequence set forth in SEQ ID NO: 212 (or a variant thereof), an HCDR2 comprising the amino acid sequence set forth in SEQ ID NO: 213 (or a variant thereof), and an HCDR3 comprising the amino acid sequence set forth in SEQ ID NO: 214 (or a variant thereof); and a LCVR that comprises: an LCDR1 comprising the amino acid sequence set forth in SEQ ID NO: 217 (or a variant thereof), an LCDR2 comprising the amino acid sequence set forth in SEQ ID NO: 218 (or a variant thereof), and an LCDR3 comprising the amino acid sequence set forth in SEQ ID NO: 219 (or a variant thereof); (f) a HCVR that comprises: an HCDR1 comprising the amino acid sequence set forth in SEQ ID NO: 222 (or a variant thereof), an HCDR2 comprising the amino acid sequence set forth in SEQ ID NO: 223 (or a variant thereof), and an HCDR3 comprising the amino acid sequence set forth in SEQ ID NO: 224 (or a variant thereof); and a LCVR that comprises: an LCDR1 comprising the amino acid sequence set forth in SEQ ID NO: 227 (or a variant thereof), an LCDR2 comprising the amino acid sequence set forth in SEQ ID NO: 228 (or a variant thereof), and an LCDR3 comprising the amino acid sequence set forth in SEQ ID NO: 229 (or a variant thereof); (g) a HCVR that comprises: an HCDR1 comprising the amino acid sequence set forth in SEQ ID NO: 232 (or a variant thereof), an HCDR2 comprising the amino acid sequence set forth in SEQ ID NO: 233 (or a variant thereof), and an HCDR3 comprising the amino acid sequence set forth in SEQ ID NO: 234 (or a variant thereof); and a LCVR that comprises: an LCDR1 comprising the amino acid sequence set forth in SEQ ID NO: 237 (or a variant thereof), an LCDR2 comprising the amino acid sequence set forth in SEQ ID NO: 238 (or a variant thereof), and an LCDR3 comprising the amino acid sequence set forth in SEQ ID NO: 239 (or a variant thereof); (h) a HCVR that comprises: an HCDR1 comprising the amino acid sequence set forth in SEQ ID NO: 242 (or a variant thereof), an HCDR2 comprising the amino acid sequence set forth in SEQ ID NO: 243 (or a variant thereof), and an HCDR3 comprising the amino acid sequence set forth in SEQ ID NO: 244 (or a variant thereof); and a LCVR that comprises: an LCDR1 comprising the amino acid sequence set forth in SEQ ID NO: 247 (or a variant thereof), an LCDR2 comprising the amino acid sequence set forth in SEQ ID NO: 248 (or a variant thereof), and an LCDR3 comprising the amino acid sequence set forth in SEQ ID NO: 249 (or a variant thereof); (i) a HCVR that comprises: an HCDR1 comprising the amino acid sequence set forth in SEQ ID NO: 252 (or a variant thereof), an HCDR2 comprising the amino acid sequence set forth in SEQ ID NO: 253 (or a variant thereof), and an HCDR3 comprising the amino acid sequence set forth in SEQ ID NO: 254 (or a variant thereof); and a LCVR that comprises: an LCDR1 comprising the amino acid sequence set forth in SEQ ID NO: 257 (or a variant thereof), an LCDR2 comprising the amino acid sequence set forth in SEQ ID NO: 258 (or a variant thereof), and an LCDR3 comprising the amino acid sequence set forth in SEQ ID NO: 259 (or a variant thereof); (j) a HCVR that comprises: an HCDR1 comprising the amino acid sequence set forth in SEQ ID NO: 262 (or a variant thereof), an HCDR2 comprising the amino acid sequence set forth in SEQ ID NO: 263 (or a variant thereof), and an HCDR3 comprising the amino acid sequence set forth in SEQ ID NO: 264 (or a variant thereof); and a LCVR that comprises: an LCDR1 comprising the amino acid sequence set forth in SEQ ID NO: 267 (or a variant thereof), an LCDR2 comprising the amino acid sequence set forth in SEQ ID NO: 268 (or a variant thereof), and an LCDR3 comprising the amino acid sequence set forth in SEQ ID NO: 269 (or a variant thereof); (k) a HCVR that comprises: an HCDR1 comprising the amino acid sequence set forth in SEQ ID NO: 272 (or a variant thereof), an HCDR2 comprising the amino acid sequence set forth in SEQ ID NO: 273 (or a variant thereof), and an HCDR3 comprising the amino acid sequence set forth in SEQ ID NO: 274 (or a variant thereof); and a LCVR that comprises: an LCDR1 comprising the amino acid sequence set forth in SEQ ID NO: 277 (or a variant thereof), an LCDR2 comprising the amino acid sequence set forth in SEQ ID NO: 278 (or a variant thereof), and an LCDR3 comprising the amino acid sequence set forth in SEQ ID NO: 279 (or a variant thereof); (1) a HCVR that comprises: an HCDR1 comprising the amino acid sequence set forth in SEQ ID NO: 282 (or a variant thereof), an HCDR2 comprising the amino acid sequence set forth in SEQ ID NO: 283 (or a variant thereof), and an HCDR3 comprising the amino acid sequence set forth in SEQ ID NO: 284 (or a variant thereof); and a LCVR that comprises: an LCDR1 comprising the amino acid sequence set forth in SEQ ID NO: 287 (or a variant thereof), an LCDR2 comprising the amino acid sequence set forth in SEQ ID NO: 288 (or a variant thereof), and an LCDR3 comprising the amino acid sequence set forth in SEQ ID NO: 289 (or a variant thereof); (m) a HCVR that comprises: an HCDR1 comprising the amino acid sequence set forth in SEQ ID NO: 292 (or a variant thereof), an HCDR2 comprising the amino acid sequence set forth in SEQ ID NO: 293 (or a variant thereof), and an HCDR3 comprising the amino acid sequence set forth in SEQ ID NO: 294 (or a variant thereof); and a LCVR that comprises: an LCDR1 comprising the amino acid sequence set forth in SEQ ID NO: 297 (or a variant thereof), an LCDR2 comprising the amino acid sequence set forth in SEQ ID NO: 298 (or a variant thereof), and an LCDR3 comprising the amino acid sequence set forth in SEQ ID NO: 299 (or a variant thereof); (n) a HCVR that comprises: an HCDR1 comprising the amino acid sequence set forth in SEQ ID NO: 302 (or a variant thereof), an HCDR2 comprising the amino acid sequence set forth in SEQ ID NO: 303 (or a variant thereof), and an HCDR3 comprising the amino acid sequence set forth in SEQ ID NO: 304 (or a variant thereof); and a LCVR that comprises: an LCDR1 comprising the amino acid sequence set forth in SEQ ID NO: 307 (or a variant thereof), an LCDR2 comprising the amino acid sequence set forth in SEQ ID NO: 308 (or a variant thereof), and an LCDR3 comprising the amino acid sequence set forth in SEQ ID NO: 309 (or a variant thereof); (o) a HCVR that comprises: an HCDR1 comprising the amino acid sequence set forth in SEQ ID NO: 312 (or a variant thereof), an HCDR2 comprising the amino acid sequence set forth in SEQ ID NO: 313 (or a variant thereof), and an HCDR3 comprising the amino acid sequence set forth in SEQ ID NO: 314 (or a variant thereof); and a LCVR that comprises: an LCDR1 comprising the amino acid sequence set forth in SEQ ID NO: 317 (or a variant thereof), an LCDR2 comprising the amino acid sequence set forth in SEQ ID NO: 318 (or a variant thereof), and an LCDR3 comprising the amino acid sequence set forth in SEQ ID NO: 319 (or a variant thereof); (p) a HCVR that comprises: an HCDR1 comprising the amino acid sequence set forth in SEQ ID NO: 322 (or a variant thereof), an HCDR2 comprising the amino acid sequence set forth in SEQ ID NO: 323 (or a variant thereof), and an HCDR3 comprising the amino acid sequence set forth in SEQ ID NO: 324 (or a variant thereof); and a LCVR that comprises: an LCDR1 comprising the amino acid sequence set forth in SEQ ID NO: 327 (or a variant thereof), an LCDR2 comprising the amino acid sequence set forth in SEQ ID NO: 328 (or a variant thereof), and an LCDR3 comprising the amino acid sequence set forth in SEQ ID NO: 329 (or a variant thereof); (q) a HCVR that comprises: an HCDR1 comprising the amino acid sequence set forth in SEQ ID NO: 332 (or a variant thereof), an HCDR2 comprising the amino acid sequence set forth in SEQ ID NO: 333 (or a variant thereof), and an HCDR3 comprising the amino acid sequence set forth in SEQ ID NO: 334 (or a variant thereof); and a LCVR that comprises: an LCDR1 comprising the amino acid sequence set forth in SEQ ID NO: 337 (or a variant thereof), an LCDR2 comprising the amino acid sequence set forth in SEQ ID NO: 338 (or a variant thereof), and an LCDR3 comprising the amino acid sequence set forth in SEQ ID NO: 339 (or a variant thereof); (r) a HCVR that comprises: an HCDR1 comprising the amino acid sequence set forth in SEQ ID NO: 342 (or a variant thereof), an HCDR2 comprising the amino acid sequence set forth in SEQ ID NO: 343 (or a variant thereof), and an HCDR3 comprising the amino acid sequence set forth in SEQ ID NO: 344 (or a variant thereof); and a LCVR that comprises: an LCDR1 comprising the amino acid sequence set forth in SEQ ID NO: 347 (or a variant thereof), an LCDR2 comprising the amino acid sequence set forth in SEQ ID NO: 348 (or a variant thereof), and an LCDR3 comprising the amino acid sequence set forth in SEQ ID NO: 349 (or a variant thereof); (s) a HCVR that comprises: an HCDR1 comprising the amino acid sequence set forth in SEQ ID NO: 352 (or a variant thereof), an HCDR2 comprising the amino acid sequence set forth in SEQ ID NO: 353 (or a variant thereof), and an HCDR3 comprising the amino acid sequence set forth in SEQ ID NO: 354 (or a variant thereof); and a LCVR that comprises: an LCDR1 comprising the amino acid sequence set forth in SEQ ID NO: 357 (or a variant thereof), an LCDR2 comprising the amino acid sequence set forth in SEQ ID NO: 358 (or a variant thereof), and an LCDR3 comprising the amino acid sequence set forth in SEQ ID NO: 359 (or a variant thereof); (t) a HCVR that comprises: an HCDR1 comprising the amino acid sequence set forth in SEQ ID NO: 362 (or a variant thereof), an HCDR2 comprising the amino acid sequence set forth in SEQ ID NO: 363 (or a variant thereof), and an HCDR3 comprising the amino acid sequence set forth in SEQ ID NO: 364 (or a variant thereof); and a LCVR that comprises: an LCDR1 comprising the amino acid sequence set forth in SEQ ID NO: 367 (or a variant thereof), an LCDR2 comprising the amino acid sequence set forth in SEQ ID NO: 368 (or a variant thereof), and an LCDR3 comprising the amino acid sequence set forth in SEQ ID NO: 369 (or a variant thereof); (u) a HCVR that comprises: an HCDR1 comprising the amino acid sequence set forth in SEQ ID NO: 372 (or a variant thereof), an HCDR2 comprising the amino acid sequence set forth in SEQ ID NO: 373 (or a variant thereof), and an HCDR3 comprising the amino acid sequence set forth in SEQ ID NO: 374 (or a variant thereof); and a LCVR that comprises: an LCDR1 comprising the amino acid sequence set forth in SEQ ID NO: 377 (or a variant thereof), an LCDR2 comprising the amino acid sequence set forth in SEQ ID NO: 378 (or a variant thereof), and an LCDR3 comprising the amino acid sequence set forth in SEQ ID NO: 379 (or a variant thereof); (v) a HCVR that comprises: an HCDR1 comprising the amino acid sequence set forth in SEQ ID NO: 382 (or a variant thereof), an HCDR2 comprising the amino acid sequence set forth in SEQ ID NO: 383 (or a variant thereof), and an HCDR3 comprising the amino acid sequence set forth in SEQ ID NO: 384 (or a variant thereof); and a LCVR that comprises: an LCDR1 comprising the amino acid sequence set forth in SEQ ID NO: 387 (or a variant thereof), an LCDR2 comprising the amino acid sequence set forth in SEQ ID NO: 388 (or a variant thereof), and an LCDR3 comprising the amino acid sequence set forth in SEQ ID NO: 389 (or a variant thereof); (w) a HCVR that comprises: an HCDR1 comprising the amino acid sequence set forth in SEQ ID NO: 392 (or a variant thereof), an HCDR2 comprising the amino acid sequence set forth in SEQ ID NO: 393 (or a variant thereof), and an HCDR3 comprising the amino acid sequence set forth in SEQ ID NO: 394 (or a variant thereof); and a LCVR that comprises: an LCDR1 comprising the amino acid sequence set forth in SEQ ID NO: 397 (or a variant thereof), an LCDR2 comprising the amino acid sequence set forth in SEQ ID NO: 398 (or a variant thereof), and an LCDR3 comprising the amino acid sequence set forth in SEQ ID NO: 399 (or a variant thereof); (x) a HCVR that comprises: an HCDR1 comprising the amino acid sequence set forth in SEQ ID NO: 402 (or a variant thereof), an HCDR2 comprising the amino acid sequence set forth in SEQ ID NO: 403 (or a variant thereof), and an HCDR3 comprising the amino acid sequence set forth in SEQ ID NO: 404 (or a variant thereof); and a LCVR that comprises: an LCDR1 comprising the amino acid sequence set forth in SEQ ID NO: 407 (or a variant thereof), an LCDR2 comprising the amino acid sequence set forth in SEQ ID NO: 408 (or a variant thereof), and an LCDR3 comprising the amino acid sequence set forth in SEQ ID NO: 409 (or a variant thereof); (y) a HCVR that comprises: an HCDR1 comprising the amino acid sequence set forth in SEQ ID NO: 412 (or a variant thereof), an HCDR2 comprising the amino acid sequence set forth in SEQ ID NO: 413 (or a variant thereof), and an HCDR3 comprising the amino acid sequence set forth in SEQ ID NO: 414 (or a variant thereof); and a LCVR that comprises: an LCDR1 comprising the amino acid sequence set forth in SEQ ID NO: 417 (or a variant thereof), an LCDR2 comprising the amino acid sequence set forth in SEQ ID NO: 418 (or a variant thereof), and an LCDR3 comprising the amino acid sequence set forth in SEQ ID NO: 419 (or a variant thereof); (z) a HCVR that comprises: an HCDR1 comprising the amino acid sequence set forth in SEQ ID NO: 422 (or a variant thereof), an HCDR2 comprising the amino acid sequence set forth in SEQ ID NO: 423 (or a variant thereof), and an HCDR3 comprising the amino acid sequence set forth in SEQ ID NO: 424 (or a variant thereof); and a LCVR that comprises: an LCDR1 comprising the amino acid sequence set forth in SEQ ID NO: 427 (or a variant thereof), an LCDR2 comprising the amino acid sequence set forth in SEQ ID NO: 428 (or a variant thereof), and an LCDR3 comprising the amino acid sequence set forth in SEQ ID NO: 429 (or a variant thereof); (aa) a HCVR that comprises: an HCDR1 comprising the amino acid sequence set forth in SEQ ID NO: 432 (or a variant thereof), an HCDR2 comprising the amino acid sequence set forth in SEQ ID NO: 433 (or a variant thereof), and an HCDR3 comprising the amino acid sequence set forth in SEQ ID NO: 434 (or a variant thereof); and a LCVR that comprises: an LCDR1 comprising the amino acid sequence set forth in SEQ ID NO: 437 (or a variant thereof), an LCDR2 comprising the amino acid sequence set forth in SEQ ID NO: 438 (or a variant thereof), and an LCDR3 comprising the amino acid sequence set forth in SEQ ID NO: 439 (or a variant thereof); (ab) a HCVR that comprises: an HCDR1 comprising the amino acid sequence set forth in SEQ ID NO: 442 (or a variant thereof), an HCDR2 comprising the amino acid sequence set forth in SEQ ID NO: 443 (or a variant thereof), and an HCDR3 comprising the amino acid sequence set forth in SEQ ID NO: 444 (or a variant thereof); and a LCVR that comprises: an LCDR1 comprising the amino acid sequence set forth in SEQ ID NO: 447 (or a variant thereof), an LCDR2 comprising the amino acid sequence set forth in SEQ ID NO: 448 (or a variant thereof), and an LCDR3 comprising the amino acid sequence set forth in SEQ ID NO: 449 (or a variant thereof); (ac) a HCVR that comprises: an HCDR1 comprising the amino acid sequence set forth in SEQ ID NO: 452 (or a variant thereof), an HCDR2 comprising the amino acid sequence set forth in SEQ ID NO: 453 (or a variant thereof), and an HCDR3 comprising the amino acid sequence set forth in SEQ ID NO: 454 (or a variant thereof); and a LCVR that comprises: an LCDR1 comprising the amino acid sequence set forth in SEQ ID NO: 457 (or a variant thereof), an LCDR2 comprising the amino acid sequence set forth in SEQ ID NO: 458 (or a variant thereof), and an LCDR3 comprising the amino acid sequence set forth in SEQ ID NO: 459 (or a variant thereof); (ad) a HCVR that comprises: an HCDR1 comprising the amino acid sequence set forth in SEQ ID NO: 462 (or a variant thereof), an HCDR2 comprising the amino acid sequence set forth in SEQ ID NO: 463 (or a variant thereof), and an HCDR3 comprising the amino acid sequence set forth in SEQ ID NO: 464 (or a variant thereof); and a LCVR that comprises: an LCDR1 comprising the amino acid sequence set forth in SEQ ID NO: 467 (or a variant thereof), an LCDR2 comprising the amino acid sequence set forth in SEQ ID NO: 468 (or a variant thereof), and an LCDR3 comprising the amino acid sequence set forth in SEQ ID NO: 469 (or a variant thereof); (ae) a HCVR that comprises: an HCDR1 comprising the amino acid sequence set forth in SEQ ID NO: 472 (or a variant thereof), an HCDR2 comprising the amino acid sequence set forth in SEQ ID NO: 473 (or a variant thereof), and an HCDR3 comprising the amino acid sequence set forth in SEQ ID NO: 474 (or a variant thereof); and a LCVR that comprises: an LCDR1 comprising the amino acid sequence set forth in SEQ ID NO: 477 (or a variant thereof), an LCDR2 comprising the amino acid sequence set forth in SEQ ID NO: 478 (or a variant thereof), and an LCDR3 comprising the amino acid sequence set forth in SEQ ID NO: 479 (or a variant thereof); and/or (af) a HCVR that comprises: an HCDR1 comprising the amino acid sequence set forth in SEQ ID NO: 482 (or a variant thereof), an HCDR2 comprising the amino acid sequence set forth in SEQ ID NO: 483 (or a variant thereof), and an HCDR3 comprising the amino acid sequence set forth in SEQ ID NO: 484 (or a variant thereof); and a LCVR that comprises: an LCDR1 comprising the amino acid sequence set forth in SEQ ID NO: 487 (or a variant thereof), an LCDR2 comprising the amino acid sequence set forth in SEQ ID NO: 488 (or a variant thereof), and an LCDR3 comprising the amino acid sequence set forth in SEQ ID NO: 489 (or a variant thereof).

In some such compositions, the anti-TfR antigen binding protein comprises: (a) a HCVR that comprises: an HCDR1 comprising the amino acid sequence set forth in SEQ ID NO: 392 (or a variant thereof), an HCDR2 comprising the amino acid sequence set forth in SEQ ID NO: 393 (or a variant thereof), and an HCDR3 comprising the amino acid sequence set forth in SEQ ID NO: 394 (or a variant thereof); and a LCVR that comprises: an LCDR1 comprising the amino acid sequence set forth in SEQ ID NO: 397 (or a variant thereof), an LCDR2 comprising the amino acid sequence set forth in SEQ ID NO: 398 (or a variant thereof), and an LCDR3 comprising the amino acid sequence set forth in SEQ ID NO: 399 (or a variant thereof); or (b) a HCVR that comprises: an HCDR1 comprising the amino acid sequence set forth in SEQ ID NO: 412 (or a variant thereof), an HCDR2 comprising the amino acid sequence set forth in SEQ ID NO: 413 (or a variant thereof), and an HCDR3 comprising the amino acid sequence set forth in SEQ ID NO: 414 (or a variant thereof); and a LCVR that comprises: an LCDR1 comprising the amino acid sequence set forth in SEQ ID NO: 417 (or a variant thereof), an LCDR2 comprising the amino acid sequence set forth in SEQ ID NO: 418 (or a variant thereof), and an LCDR3 comprising the amino acid sequence set forth in SEQ ID NO: 419 (or a variant thereof). In some such compositions, the anti-TfR antigen binding protein comprises: a HCVR that comprises: an HCDR1 comprising the amino acid sequence set forth in SEQ ID NO: 392 (or a variant thereof), an HCDR2 comprising the amino acid sequence set forth in SEQ ID NO: 393 (or a variant thereof), and an HCDR3 comprising the amino acid sequence set forth in SEQ ID NO: 394 (or a variant thereof); and a LCVR that comprises: an LCDR1 comprising the amino acid sequence set forth in SEQ ID NO: 397 (or a variant thereof), an LCDR2 comprising the amino acid sequence set forth in SEQ ID NO: 398 (or a variant thereof), and an LCDR3 comprising the amino acid sequence set forth in SEQ ID NO: 399 (or a variant thereof).

In some such compositions, the anti-TfR antigen binding protein comprises: (i) a HCVR that comprises the amino acid sequence set forth in SEQ ID NO: 171 (or a variant thereof); and a LCVR that comprises the amino acid sequence set forth in SEQ ID NO: 176 (or a variant thereof); (ii) a HCVR that comprises the amino acid sequence set forth in SEQ ID NO: 181 (or a variant thereof); and a LCVR that comprises the amino acid sequence set forth in SEQ ID NO: 186 (or a variant thereof); (iii) a HCVR that comprises the amino acid sequence set forth in SEQ ID NO: 191 (or a variant thereof); and a LCVR that comprises the amino acid sequence set forth in SEQ ID NO: 196 (or a variant thereof); (iv) a HCVR that comprises the amino acid sequence set forth in SEQ ID NO: 201 (or a variant thereof); and a LCVR that comprises the amino acid sequence set forth in SEQ ID NO: 206 (or a variant thereof); (v) a HCVR that comprises the amino acid sequence set forth in SEQ ID NO: 211 (or a variant thereof); and a LCVR that comprises the amino acid sequence set forth in SEQ ID NO: 216 (or a variant thereof); (vi) a HCVR that comprises the amino acid sequence set forth in SEQ ID NO: 221 (or a variant thereof); and a LCVR that comprises the amino acid sequence set forth in SEQ ID NO: 226 (or a variant thereof); (vii) a HCVR that comprises the amino acid sequence set forth in SEQ ID NO: 231 (or a variant thereof); and a LCVR that comprises the amino acid sequence set forth in SEQ ID NO: 236 (or a variant thereof); (viii) a HCVR that comprises the amino acid sequence set forth in SEQ ID NO: 241 (or a variant thereof); and a LCVR that comprises the amino acid sequence set forth in SEQ ID NO: 246 (or a variant thereof); (ix) a HCVR that comprises the amino acid sequence set forth in SEQ ID NO: 251 (or a variant thereof); and a LCVR that comprises the amino acid sequence set forth in SEQ ID NO: 256 (or a variant thereof); (x) a HCVR that comprises the amino acid sequence set forth in SEQ ID NO: 261 (or a variant thereof); and a LCVR that comprises the amino acid sequence set forth in SEQ ID NO: 266 (or a variant thereof); (xi) a HCVR that comprises the amino acid sequence set forth in SEQ ID NO: 271 (or a variant thereof); and a LCVR that comprises the amino acid sequence set forth in SEQ ID NO: 276 (or a variant thereof); (xii) a HCVR that comprises the amino acid sequence set forth in SEQ ID NO: 281 (or a variant thereof); and a LCVR that comprises the amino acid sequence set forth in SEQ ID NO: 286 (or a variant thereof); (xiii) a HCVR that comprises the amino acid sequence set forth in SEQ ID NO: 291 (or a variant thereof); and a LCVR that comprises the amino acid sequence set forth in SEQ ID NO: 296 (or a variant thereof); (xiv) a HCVR that comprises the amino acid sequence set forth in SEQ ID NO: 301 (or a variant thereof); and a LCVR that comprises the amino acid sequence set forth in SEQ ID NO: 306 (or a variant thereof); (xv) a HCVR that comprises the amino acid sequence set forth in SEQ ID NO: 311 (or a variant thereof); and a LCVR that comprises the amino acid sequence set forth in SEQ ID NO: 316 (or a variant thereof); (xvi) a HCVR that comprises the amino acid sequence set forth in SEQ ID NO: 321 (or a variant thereof); and a LCVR that comprises the amino acid sequence set forth in SEQ ID NO: 326 (or a variant thereof); (xvii) a HCVR that comprises the amino acid sequence set forth in SEQ ID NO: 331 (or a variant thereof); and a LCVR that comprises the amino acid sequence set forth in SEQ ID NO: 336 (or a variant thereof); (xviii) a HCVR that comprises the amino acid sequence set forth in SEQ ID NO: 341 (or a variant thereof); and a LCVR that comprises the amino acid sequence set forth in SEQ ID NO: 346 (or a variant thereof); (xix) a HCVR that comprises the amino acid sequence set forth in SEQ ID NO: 351 (or a variant thereof); and a LCVR that comprises the amino acid sequence set forth in SEQ ID NO: 356 (or a variant thereof); (xx) a HCVR that comprises the amino acid sequence set forth in SEQ ID NO: 361 (or a variant thereof); and a LCVR that comprises the amino acid sequence set forth in SEQ ID NO: 366 (or a variant thereof); (xxi) a HCVR that comprises the amino acid sequence set forth in SEQ ID NO: 371 (or a variant thereof); and a LCVR that comprises the amino acid sequence set forth in SEQ ID NO: 376 (or a variant thereof); (xxii) a HCVR that comprises the amino acid sequence set forth in SEQ ID NO: 381 (or a variant thereof); and a LCVR that comprises the amino acid sequence set forth in SEQ ID NO: 386 (or a variant thereof); (xxiii) a HCVR that comprises the amino acid sequence set forth in SEQ ID NO: 391 (or a variant thereof); and a LCVR that comprises the amino acid sequence set forth in SEQ ID NO: 396 (or a variant thereof); (xxiv) a HCVR that comprises the amino acid sequence set forth in SEQ ID NO: 401 (or a variant thereof); and a LCVR that comprises the amino acid sequence set forth in SEQ ID NO: 406 (or a variant thereof); (xxv) a HCVR that comprises the amino acid sequence set forth in SEQ ID NO: 411 (or a variant thereof); and a LCVR that comprises the amino acid sequence set forth in SEQ ID NO: 416 (or a variant thereof); (xxvi) a HCVR that comprises the amino acid sequence set forth in SEQ ID NO: 421 (or a variant thereof); and a LCVR that comprises the amino acid sequence set forth in SEQ ID NO: 426 (or a variant thereof); (xxvii) a HCVR that comprises the amino acid sequence set forth in SEQ ID NO: 431 (or a variant thereof); and a LCVR that comprises the amino acid sequence set forth in SEQ ID NO: 436 (or a variant thereof); (xxviii) a HCVR that comprises the amino acid sequence set forth in SEQ ID NO: 441 (or a variant thereof); and a LCVR that comprises the amino acid sequence set forth in SEQ ID NO: 446 (or a variant thereof); (xxix) a HCVR that comprises the amino acid sequence set forth in SEQ ID NO: 451 (or a variant thereof); and a LCVR that comprises the amino acid sequence set forth in SEQ ID NO: 456 (or a variant thereof); (xxx) a HCVR that comprises the amino acid sequence set forth in SEQ ID NO: 461 (or a variant thereof); and a LCVR that comprises the amino acid sequence set forth in SEQ ID NO: 466 (or a variant thereof); (xxxi) a HCVR that comprises the amino acid sequence set forth in SEQ ID NO: 471 (or a variant thereof); and a LCVR that comprises the amino acid sequence set forth in SEQ ID NO: 476 (or a variant thereof); and/or (xxxii) a HCVR that comprises the amino acid sequence set forth in SEQ ID NO: 481 (or a variant thereof); and a LCVR that comprises the amino acid sequence set forth in SEQ ID NO: 486 (or a variant thereof).

In some such compositions, the anti-TfR antigen binding protein comprises: (i) a HCVR that comprises the amino acid sequence set forth in SEQ ID NO: 391 (or a variant thereof); and a LCVR that comprises the amino acid sequence set forth in SEQ ID NO: 396 (or a variant thereof); or (ii) a HCVR that comprises the amino acid sequence set forth in SEQ ID NO: 411 (or a variant thereof); and a LCVR that comprises the amino acid sequence set forth in SEQ ID NO: 416 (or a variant thereof). In some such compositions, the anti-TfR antigen binding protein comprises: a HCVR that comprises the amino acid sequence set forth in SEQ ID NO: 391 (or a variant thereof); and a LCVR that comprises the amino acid sequence set forth in SEQ ID NO: 396 (or a variant thereof).

In some such compositions, the TfR-binding delivery domain is an antigen-binding protein that binds to one or more epitopes of hTfR selected from: (a) an epitope comprising the sequence LLNE (SEQ ID NO: 796) and/or an epitope comprising the sequence TYKEL (SEQ ID NO: 706); (b) an epitope comprising the sequence DSTDFTGT (SEQ ID NO: 797) and/or an epitope comprising the sequence VKHPVTGQF (SEQ ID NO: 798) and/or an epitope comprising the sequence IERIPEL (SEQ ID NO: 799); (c) an epitope comprising the sequence LNENSYVPREAGSQKDEN (SEQ ID NO: 800); (d) an epitope comprising the sequence FEDL (SEQ ID NO: 718); (e) an epitope comprising the sequence IVDKNGRL (SEQ ID NO: 801); (f) an epitope comprising the sequence IVDKNGRLVY (SEQ ID NO: 802); (g) an epitope comprising the sequence DQTKF (SEQ ID NO: 803); (h) an epitope comprising the sequence LVENPGGY (SEQ ID NO: 804) and/or an epitope comprising the sequence PIVNAELSF (SEQ ID NO: 805) and/or an epitope comprising the sequence PYLGTTMDT (SEQ ID NO: 806); (i) an epitope comprising the sequence LLNENSYVPREAGSQKDENLAL (SEQ ID NO: 704) and/or an epitope comprising the sequence IYMDQTKFPIVNAEL (SEQ ID NO: 705) and/or an epitope comprising the sequence TYKEL (SEQ ID NO: 706); (j) an epitope comprising the sequence KRKLSEKLDSTDFTGTIKL (SEQ ID NO: 707) and/or an epitope comprising the sequence YTLIEKTMQNVKHPVTGQFL (SEQ ID NO: 708) and/or an epitope comprising the sequence LIERIPELNKVARAAAE (SEQ ID NO: 709); (k) an epitope comprising the sequence LNENSYVPREAGSQKDENL (SEQ ID NO: 710); (1) an epitope comprising the sequence GTKKDFEDL (SEQ ID NO: 711); (m) an epitope comprising the sequence SVIIVDKNGRLVYLVENPGGYVAYSK (SEQ ID NO: 712); (n) an epitope comprising the sequence LLNENSYVPREAGSQKDEN (SEQ ID NO: 713) and/or an epitope comprising the sequence DQTKFPIVNAEL (SEQ ID NO: 714) and/or an epitope comprising the sequence TYKELIERIPELNK (SEQ ID NO: 715); (o) an epitope comprising the sequence LLNENSYVPREAGSQKDEN (SEQ ID NO: 713) and/or an epitope comprising the sequence TYKELIERIPELNK (SEQ ID NO: 715); (p) an epitope comprising the sequence SVIIVDKNGRLVYLVENPGGYVAY (SEQ ID NO: 716); (q) an epitope comprising the sequence IYMDQTKFPIVNAEL (SEQ ID NO: 705) and/or an epitope comprising the sequence FGNMEGDCPSDWKTDSTCRM (SEQ ID NO: 717); (r) an epitope comprising the sequence LLNENSYVPREAGSQKDENLAL (SEQ ID NO: 704) and/or an epitope comprising the sequence LVENPGGYVAYSKAATVTGKL (SEQ ID NO: 719) and/or an epitope comprising the sequence IYMDQTKFPIVNAELSF (SEQ ID NO: 720) and/or an epitope comprising the sequence ISRAAAEKL (SEQ ID NO: 721) and/or an epitope comprising the sequence VTSESKNVKLTVSNVLKE (SEQ ID NO: 722) and/or an epitope comprising the sequence FCEDTDYPYLGTTMDT (SEQ ID NO: 723); (s) an epitope comprised within or overlapping with the sequence LLNENSYVPREAGSQKDENLAL (SEQ ID NO: 704) and/or an epitope comprised within or overlapping with the sequence IYMDQTKFPIVNAEL (SEQ ID NO: 705) and/or an epitope comprised within or overlapping with the sequence TYKEL (SEQ ID NO: 706); (t) an epitope comprised within or overlapping with the sequence KRKLSEKLDSTDFTGTIKL (SEQ ID NO: 707) and/or an epitope comprised within or overlapping with the sequence YTLIEKTMQNVKHPVTGQFL (SEQ ID NO: 708) and/or an epitope comprised within or overlapping with the sequence LIERIPELNKVARAAAE (SEQ ID NO: 709); (u) an epitope comprised within or overlapping with the sequence LNENSYVPREAGSQKDENL (SEQ ID NO: 710); (v) an epitope comprised within or overlapping with the sequence GTKKDFEDL (SEQ ID NO: 711); (w) an epitope comprised within or overlapping with the sequence SVIIVDKNGRLVYLVENPGGYVAYSK (SEQ ID NO: 712); (x) an epitope comprised within or overlapping with the sequence LLNENSYVPREAGSQKDEN (SEQ ID NO: 713) and/or an epitope comprised within or overlapping with the sequence DQTKFPIVNAEL (SEQ ID NO: 714) and/or an epitope comprised within or overlapping with the sequence TYKELIERIPELNK (SEQ ID NO: 715); (y) an epitope comprised within or overlapping with the sequence LLNENSYVPREAGSQKDEN (SEQ ID NO: 713) and/or an epitope comprised within or overlapping with the sequence TYKELIERIPELNK (SEQ ID NO: 715); (z) an epitope comprised within or overlapping with the sequence SVIIVDKNGRLVYLVENPGGYVAY (SEQ ID NO: 716); (aa) an epitope comprised within or overlapping with the sequence IYMDQTKFPIVNAEL (SEQ ID NO: 705) and/or an epitope comprised within or overlapping with the sequence FGNMEGDCPSDWKTDSTCRM (SEQ ID NO: 717); and (ab) an epitope comprised within or overlapping with the sequence LLNENSYVPREAGSQKDENLAL (SEQ ID NO: 704) and/or an epitope comprised within or overlapping with the sequence LVENPGGYVAYSKAATVTGKL (SEQ ID NO: 719) and/or an epitope comprised within or overlapping with the sequence IYMDQTKFPIVNAELSF (SEQ ID NO: 720) and/or an epitope comprised within or overlapping with the sequence ISRAAAEKL (SEQ ID NO: 721) and/or an epitope comprised within or overlapping with the sequence VTSESKNVKLTVSNVLKE (SEQ ID NO: 722) and/or an epitope comprised within or overlapping with the sequence FCEDTDYPYLGTTMDT (SEQ ID NO: 723).

In some such compositions, the TfR-binding delivery domain comprises an antibody or antigen-binding fragment thereof that binds to one or more epitopes of hTfR selected from: (a) an epitope consisting of the sequence LLNE (SEQ ID NO: 796) and/or an epitope consisting of the sequence TYKEL (SEQ ID NO: 706); (b) an epitope consisting of the sequence DSTDFTGT (SEQ ID NO: 797) and/or an epitope consisting of the sequence VKHPVTGQF (SEQ ID NO: 798) and/or an epitope consisting of the sequence IERIPEL (SEQ ID NO: 799); (c) an epitope consisting of the sequence LNENSYVPREAGSQKDEN (SEQ ID NO: 800); (d) an epitope consisting of the sequence FEDL (SEQ ID NO: 718); (e) an epitope consisting of the sequence IVDKNGRL (SEQ ID NO: 801); (f) an epitope consisting of the sequence IVDKNGRLVY (SEQ ID NO: 802); (g) an epitope consisting of the sequence DQTKF (SEQ ID NO: 803); (h) an epitope consisting of the sequence LVENPGGY (SEQ ID NO: 804) and/or an epitope consisting of the sequence PIVNAELSF (SEQ ID NO: 805) and/or an epitope consisting of the sequence PYLGTTMDT (SEQ ID NO: 806); (i) an epitope consisting of the sequence LLNENSYVPREAGSQKDENLAL (SEQ ID NO: 704) and/or an epitope consisting of the sequence IYMDQTKFPIVNAEL (SEQ ID NO: 705) and/or an epitope consisting of the sequence TYKEL (SEQ ID NO: 706); (j) an epitope consisting of the sequence KRKLSEKLDSTDFTGTIKL (SEQ ID NO: 707) and/or an epitope consisting of the sequence YTLIEKTMQNVKHPVTGQFL (SEQ ID NO: 708) and/or an epitope consisting of the sequence LIERIPELNKVARAAAE (SEQ ID NO: 709); (k) an epitope consisting of the sequence LNENSYVPREAGSQKDENL (SEQ ID NO: 710); (1) an epitope consisting of the sequence GTKKDFEDL (SEQ ID NO: 711); (m) an epitope consisting of the sequence SVIIVDKNGRLVYLVENPGGYVAYSK (SEQ ID NO: 712); (n) an epitope consisting of the sequence LLNENSYVPREAGSQKDEN (SEQ ID NO: 713) and/or an epitope consisting of the sequence DQTKFPIVNAEL (SEQ ID NO: 714) and/or an epitope consisting of the sequence TYKELIERIPELNK (SEQ ID NO: 715); (o) an epitope consisting of the sequence LLNENSYVPREAGSQKDEN (SEQ ID NO: 713) and/or an epitope consisting of the sequence TYKELIERIPELNK (SEQ ID NO: 715); (p) an epitope consisting of the sequence SVIIVDKNGRLVYLVENPGGYVAY (SEQ ID NO: 716); (q) an epitope consisting of the sequence IYMDQTKFPIVNAEL (SEQ ID NO: 705) and/or an epitope consisting of the sequence FGNMEGDCPSDWKTDSTCRM (SEQ ID NO: 717); and (r) an epitope consisting of the sequence LLNENSYVPREAGSQKDENLAL (SEQ ID NO: 704) and/or an epitope consisting of the sequence LVENPGGYVAYSKAATVTGKL (SEQ ID NO: 719) and/or an epitope consisting of the sequence IYMDQTKFPIVNAELSF (SEQ ID NO: 720) and/or an epitope consisting of the sequence ISRAAAEKL (SEQ ID NO: 721) and/or an epitope consisting of the sequence VTSESKNVKLTVSNVLKE (SEQ ID NO: 722) and/or an epitope consisting of the sequence FCEDTDYPYLGTTMDT (SEQ ID NO: 723).

In some such compositions, the TfR-binding delivery domain comprises an anti-TfR antibody, antibody fragment, or single-chain variable fragment (scFv). In some such compositions, the TfR-binding delivery domain is the single-chain variable fragment (scFv), optionally wherein the multidomain therapeutic protein comprises domains arranged in the following orientation: N′-heavy chain variable region-light chain variable region-lysosomal alpha-glucosidase polypeptide-C′ or N′-light chain variable region-heavy chain variable region-lysosomal alpha-glucosidase polypeptide-C′, optionally wherein the scFv and lysosomal alpha-glucosidase polypeptide are connected by a peptide linker, and optionally wherein the peptide linker which is -(GGGGS)ₘ- (SEQ ID NO: 537); wherein m is 1, 2, 3, 4, 5, 6, 7, 8, 9, or 10, optionally wherein the scFv variable regions are connected by a peptide linker, and optionally wherein the peptide linker which is -(GGGGS)ₘ- (SEQ ID NO: 537); wherein m is 1, 2, 3, 4, 5, 6, 7, 8, 9, or 10. In some such compositions, the multidomain therapeutic protein comprises a heavy chain variable region (V_{H}) and a light chain variable region (V_{L}), and the lysosomal alpha-glucosidase polypeptide, wherein the V_{H}, V_{L} and lysosomal alpha-glucosidase polypeptide are arranged as follows: (i) V_{L}-V_{H}-lysosomal alpha-glucosidase polypeptide; (ii) V_{H}-V_{L}-lysosomal alpha-glucosidase polypeptide; (iii) V_{L}-[(GGGGS)₃(SEQ ID NO: 616)]-V_{H}-[(GGGGS)₂(SEQ ID NO: 617)]-lysosomal alpha-glucosidase polypeptide; or (iv) V_{H}-[(GGGGS)3(SEQ ID NO: 616)]-V_{L}-[(GGGGS)₂(SEQ ID NO: 617)]-lysosomal alpha-glucosidase polypeptide.

In some such compositions, the scFv comprises, consists essentially of, or consists of the sequence set forth in SEQ ID NO: 508. In some such compositions, the scFv coding sequence is at least 90%, at least 91%, at least 92%, at least 93%, at least 94%, at least 95%, at least 96%, at least 97%, at least 98%, or at least 99% identical to SEQ ID NO: 532 and encodes an scFv comprising SEQ ID NO: 508. In some such compositions, the scFv coding sequence comprises, consists essentially of, or consists of the sequence set forth in SEQ ID NO: 532. In some such compositions, the multidomain therapeutic protein comprises, consists essentially of, or consists of the sequence set forth in SEQ ID NO: 746. In some such compositions, the multidomain therapeutic protein coding sequence is at least 90%, at least 91%, at least 92%, at least 93%, at least 94%, at least 95%, at least 96%, at least 97%, at least 98%, or at least 99% identical to SEQ ID NO: 745, optionally wherein the nucleotide at position 1857 is G, the nucleotide at position 1860 is C, and the nucleotide at position 3105 is G. In some such compositions, the multidomain therapeutic protein coding sequence is at least 90%, at least 91%, at least 92%, at least 93%, at least 94%, at least 95%, at least 96%, at least 97%, at least 98%, or at least 99% identical to SEQ ID NO: 745 and encodes a multidomain therapeutic protein comprising SEQ ID NO: 746, optionally wherein the nucleotide at position 1857 is G, the nucleotide at position 1860 is C, and the nucleotide at position 3105 is G. In some such compositions, the multidomain therapeutic protein coding sequence comprises, consists essentially of, or consists of the sequence set forth in SEQ ID NO: 745.

In some such compositions, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises SEQ ID NO: 745, optionally wherein the nucleic acid construct comprises the sequence set forth in SEQ ID NO: 780, optionally wherein the nucleic acid construct comprises the sequence set forth in SEQ ID NO: 764, wherein the polyadenylation signal comprises a BGH polyadenylation signal and a unidirectional SV40 late polyadenylation signal, optionally wherein the BGH polyadenylation signal comprises the sequence set forth in SEQ ID NO: 751 and the unidirectional SV40 late polyadenylation signal comprises the sequence set forth in SEQ ID NO: 752, optionally wherein the polyadenylation signal comprising the BGH polyadenylation signal and the unidirectional SV40 late polyadenylation signal comprises the sequence set forth in SEQ ID NO: 795, wherein the nucleic acid construct does not comprise a promoter that drives the expression of the multidomain therapeutic protein, and wherein the nucleic acid construct does not comprise a homology arm. In some such compositions, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises SEQ ID NO: 745, optionally wherein the nucleic acid construct comprises the sequence set forth in SEQ ID NO: 781, optionally wherein the nucleic acid construct comprises the sequence set forth in SEQ ID NO: 765, wherein the polyadenylation signal comprises a BGH polyadenylation signal, optionally wherein the BGH polyadenylation signal comprises the sequence set forth in SEQ ID NO: 751, wherein the nucleic acid construct does not comprise a promoter that drives the expression of the multidomain therapeutic protein, and wherein the nucleic acid construct does not comprise a homology arm.

In some such compositions, the delivery domain is the CD63-binding delivery domain. In some such compositions, the CD63-binding delivery domain comprises an anti-CD63 antigen-binding protein. In some such compositions, the CD63-binding delivery domain comprises an anti-CD63 antibody, antibody fragment, or single-chain variable fragment (scFv). In some such compositions, the CD63-binding delivery domain is the single-chain variable fragment (scFv). In some such compositions, the scFv comprises, consists essentially of, or consists of the sequence set forth in SEQ ID NO: 730. In some such compositions, the scFv coding sequence is at least 90%, at least 91%, at least 92%, at least 93%, at least 94%, at least 95%, at least 96%, at least 97%, at least 98%, or at least 99% identical to SEQ ID NO: 759, optionally wherein the nucleotide at position 3 is A, the nucleotide at position 132 is A, and the nucleotide at position 273 is T. In some such compositions, the scFv coding sequence is at least 90%, at least 91%, at least 92%, at least 93%, at least 94%, at least 95%, at least 96%, at least 97%, at least 98%, or at least 99% identical to SEQ ID NO: 759 and encodes an scFv comprising SEQ ID NO: 730, optionally wherein the nucleotide at position 3 is A, the nucleotide at position 132 is A, and the nucleotide at position 273 is T. In some such compositions, the scFv coding sequence comprises, consists essentially of, or consists of the sequence set forth in SEQ ID NO: 759. In some such compositions, the scFv coding sequence is at least 90%, at least 91%, at least 92%, at least 93%, at least 94%, at least 95%, at least 96%, at least 97%, at least 98%, or at least 99% identical to SEQ ID NO: 760, optionally wherein the nucleotide at position 273 is T. In some such compositions, the scFv coding sequence is at least 90%, at least 91%, at least 92%, at least 93%, at least 94%, at least 95%, at least 96%, at least 97%, at least 98%, or at least 99% identical to SEQ ID NO: 760 and encodes an scFv comprising SEQ ID NO: 730, optionally wherein the nucleotide at position 273 is T. In some such compositions, the scFv coding sequence comprises, consists essentially of, or consists of the sequence set forth in SEQ ID NO: 760. In some such compositions, the scFv coding sequence is at least 90%, at least 91%, at least 92%, at least 93%, at least 94%, at least 95%, at least 96%, at least 97%, at least 98%, or at least 99% identical to SEQ ID NO: 732. In some such compositions, the scFv coding sequence is at least 90%, at least 91%, at least 92%, at least 93%, at least 94%, at least 95%, at least 96%, at least 97%, at least 98%, or at least 99% identical to SEQ ID NO: 732 and encodes an scFv comprising SEQ ID NO: 730. In some such compositions, the scFv coding sequence comprises, consists essentially of, or consists of the sequence set forth in SEQ ID NO: 732.

In some such compositions, the multidomain therapeutic protein comprises, consists essentially of, or consists of the sequence set forth in SEQ ID NO: 733. In some such compositions, the multidomain therapeutic protein coding sequence is at least 90%, at least 91%, at least 92%, at least 93%, at least 94%, at least 95%, at least 96%, at least 97%, at least 98%, or at least 99% identical to SEQ ID NO: 756, optionally wherein the nucleotide at position 3 is A, the nucleotide at position 132 is A, the nucleotide at position 273 is T, the nucleotide at position 723 is G, the nucleotide at position 1830 is G, the nucleotide at position 1833 is C, and the nucleotide at position 3078 is G. In some such compositions, the multidomain therapeutic protein coding sequence is at least 90%, at least 91%, at least 92%, at least 93%, at least 94%, at least 95%, at least 96%, at least 97%, at least 98%, or at least 99% identical to SEQ ID NO: 756 and encodes a multidomain therapeutic protein comprising SEQ ID NO: 733, optionally wherein the nucleotide at position 3 is A, the nucleotide at position 132 is A, the nucleotide at position 273 is T, the nucleotide at position 723 is G, the nucleotide at position 1830 is G, the nucleotide at position 1833 is C, and the nucleotide at position 3078 is G. In some such compositions, the multidomain therapeutic protein coding sequence comprises, consists essentially of, or consists of the sequence set forth in SEQ ID NO: 756. In some such compositions, the multidomain therapeutic protein coding sequence is at least 90%, at least 91%, at least 92%, at least 93%, at least 94%, at least 95%, at least 96%, at least 97%, at least 98%, or at least 99% identical to SEQ ID NO: 757, optionally wherein the nucleotide at position 273 is T, the nucleotide at position 723 is G, the nucleotide at position 1830 is G, the nucleotide at position 1833 is C, and the nucleotide at position 3078 is G. In some such compositions, the multidomain therapeutic protein coding sequence is at least 90%, at least 91%, at least 92%, at least 93%, at least 94%, at least 95%, at least 96%, at least 97%, at least 98%, or at least 99% identical to SEQ ID NO: 757 and encodes a multidomain therapeutic protein comprising SEQ ID NO: 733, optionally wherein the nucleotide at position 273 is T, the nucleotide at position 723 is G, the nucleotide at position 1830 is G, the nucleotide at position 1833 is C, and the nucleotide at position 3078 is G. In some such compositions, the multidomain therapeutic protein coding sequence comprises, consists essentially of, or consists of the sequence set forth in SEQ ID NO: 757. In some such compositions, the multidomain therapeutic protein coding sequence is at least 90%, at least 91%, at least 92%, at least 93%, at least 94%, at least 95%, at least 96%, at least 97%, at least 98%, or at least 99% identical to SEQ ID NO: 758, optionally wherein the nucleotide at position 3078 is G. In some such compositions, the multidomain therapeutic protein coding sequence is at least 90%, at least 91%, at least 92%, at least 93%, at least 94%, at least 95%, at least 96%, at least 97%, at least 98%, or at least 99% identical to SEQ ID NO: 758 and encodes a multidomain therapeutic protein comprising SEQ ID NO: 733, optionally wherein the nucleotide at position 3078 is G. In some such compositions, the multidomain therapeutic protein coding sequence comprises, consists essentially of, or consists of the sequence set forth in SEQ ID NO: 758.

In some such compositions, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises SEQ ID NO: 756, optionally wherein the nucleic acid construct comprises the sequence set forth in SEQ ID NO: 793, optionally wherein the nucleic acid construct comprises the sequence set forth in SEQ ID NO: 777, wherein the polyadenylation signal comprises a BGH polyadenylation signal and a unidirectional SV40 late polyadenylation signal, optionally wherein the BGH polyadenylation signal comprises the sequence set forth in SEQ ID NO: 751 and the unidirectional SV40 late polyadenylation signal comprises the sequence set forth in SEQ ID NO: 752, optionally wherein the polyadenylation signal comprising the BGH polyadenylation signal and the unidirectional SV40 late polyadenylation signal comprises the sequence set forth in SEQ ID NO: 795, wherein the nucleic acid construct does not comprise a promoter that drives the expression of the multidomain therapeutic protein, and wherein the nucleic acid construct does not comprise a homology arm. In some such compositions, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises SEQ ID NO: 756, optionally wherein the nucleic acid construct comprises the sequence set forth in SEQ ID NO: 794, optionally wherein the nucleic acid construct comprises the sequence set forth in SEQ ID NO: 778, wherein the polyadenylation signal comprises a BGH polyadenylation signal, optionally wherein the BGH polyadenylation signal comprises the sequence set forth in SEQ ID NO: 751, wherein the nucleic acid construct does not comprise a promoter that drives the expression of the multidomain therapeutic protein, and wherein the nucleic acid construct does not comprise a homology arm.

In some such compositions, the nucleic acid construct is in a nucleic acid vector or a lipid nanoparticle. In some such compositions, the nucleic acid construct is in the nucleic acid vector, optionally wherein the nucleic acid vector is a viral vector. In some such compositions, the nucleic acid vector is an adeno-associated viral (AAV) vector, optionally wherein the nucleic acid construct is flanked by inverted terminal repeats (ITRs) on each end, optionally wherein the ITR on at least one end comprises, consists essentially of, or consists of SEQ ID NO: 160, and optionally wherein the ITR on each end comprises, consists essentially of, or consists of SEQ ID NO: 160. In some such compositions, the AAV vector is a single-stranded AAV (ssAAV) vector. In some such compositions, the AAV vector is a recombinant AAV8 (rAAV8) vector, optionally wherein the AAV vector is a single-stranded rAAV8 vector.

In some such compositions, the composition is in combination with a nuclease agent that targets a nuclease target site in a target genomic locus. In some such compositions, the target genomic locus is an albumin gene, optionally wherein the albumin gene is a human albumin gene. In some such compositions, the nuclease target site is in intron 1 of the albumin gene. In some such compositions, the nuclease agent comprises: (a) a zinc finger nuclease (ZFN); (b) a transcription activator-like effector nuclease (TALEN); or (c) (i) a Cas protein or a nucleic acid encoding the Cas protein; and (ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence. In some such compositions, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

In some such compositions, the guide RNA target sequence is in intron 1 of an albumin gene. In some such compositions, the DNA-targeting segment comprises any one of SEQ ID NOS: 30-61, optionally wherein the DNA-targeting segment comprises any one of SEQ ID NOS: 36, 30, 33, and 41, or wherein the DNA-targeting segment consists of any one of SEQ ID NOS: 30-61, optionally wherein the DNA-targeting segment consists of any one of SEQ ID NOS: 36, 30, 33, and 41. In some such compositions, the guide RNA comprises any one of SEQ ID NOS: 62-125, optionally wherein the guide RNA comprises any one of SEQ ID NOS: 68, 100, 62, 94, 65, 97, 73, and 105. In some such compositions, the DNA-targeting segment comprises or consists of SEQ ID NO: 36. In some such compositions, the guide RNA comprises SEQ ID NO: 68 or 100. In some such compositions, the composition comprises the guide RNA in the form of RNA. In some such compositions, the guide RNA comprises at least one modification. In some such compositions, the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. In some such compositions, the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

In some such compositions, the Cas protein is a Cas9 protein, optionally wherein the Cas protein is derived from a Streptococcus pyogenes Cas9 protein. In some such compositions, the Cas protein comprises the sequence set forth in SEQ ID NO: 11. In some such compositions, the composition comprises the nucleic acid encoding the Cas protein, wherein the nucleic acid comprises an mRNA encoding the Cas protein. In some such compositions, the mRNA encoding the Cas protein comprises at least one modification. In some such compositions, the mRNA encoding the Cas protein is fully substituted with N1-methyl-pseudouridine. In some such compositions, the mRNA encoding the Cas protein comprises the sequence set forth in SEQ ID NO: 1 or 2. In some such compositions, the composition comprises the nucleic acid encoding the Cas protein, wherein the nucleic acid comprises an mRNA encoding the Cas protein, the mRNA encoding the Cas protein comprises the sequence set forth in SEQ ID NO: 1 or 2, and the mRNA encoding the Cas protein is fully substituted with N1-methyl-pseudouridine, comprises a 5′ cap, and comprises a poly(A) tail.

In some such compositions, the composition comprises the guide RNA in the form of RNA, and the guide RNA comprises SEQ ID NO: 68 or 100, and wherein the composition comprises administering the nucleic acid encoding the Cas protein, wherein the nucleic acid comprises an mRNA encoding the Cas protein, and the mRNA encoding the Cas protein comprises the sequence set forth in SEQ ID NO: 1 or 2. In some such compositions, the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition the nucleic acid encoding the Cas protein, wherein the nucleic acid comprises an mRNA encoding the Cas protein, the mRNA encoding the Cas protein comprises the sequence set forth in SEQ ID NO: 1 or 2, and the mRNA encoding the Cas protein is fully substituted with N1-methyl-pseudouridine, comprises a 5′ cap, and comprises a poly(A) tail.

In some such compositions, the Cas protein or the nucleic acid encoding the Cas protein and the guide RNA or the one or more DNAs encoding the guide RNA are associated with a lipid nanoparticle. In some such compositions, the lipid nanoparticle comprises a cationic lipid, a neutral lipid, a helper lipid, and a stealth lipid. In some such compositions, the cationic lipid is Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate), and/or wherein the neutral lipid is distearoylphosphatidylcholine or 1,2-distearoyl-sn-glycero-3-phosphocholine (DSPC), and/or wherein the helper lipid is cholesterol, and/or wherein the stealth lipid is 1,2-dimyristoyl-rac-glycero-3-methoxypolyethylene glycol-2000. In some such compositions, the cationic lipid is Lipid A, the neutral lipid is DSPC, the helper lipid is cholesterol, and the stealth lipid is PEG2k-DMG. In some such compositions, the lipid nanoparticle comprises four lipids at the following molar ratios: about 50 mol % Lipid A, about 9 mol % DSPC, about 38 mol % cholesterol, and about 3 mol % PEG2k-DMG.

In another aspect, provided are cells comprising any of the above compositions. In some such cells, the nucleic acid construct or the coding sequence for the multidomain therapeutic protein is integrated into a target genomic locus, and wherein the multidomain therapeutic protein is expressed from the target genomic locus, or wherein the nucleic acid construct or the coding sequence for the multidomain therapeutic protein is integrated into intron 1 of an endogenous albumin locus, and wherein the multidomain therapeutic protein is expressed from the endogenous albumin locus. In some such cells, the percentage of unintended transcripts from the target genomic locus containing comprising the integrated nucleic acid construct or coding sequence for the multidomain therapeutic protein is less than about 10%, less than about 9%, less than about 8%, less than about 7%, less than about 6%, less than about 5%, less than about 4%, less than about 3%, less than about 2%, or less than about 1%. In some such cells, the cell is a liver cell or a hepatocyte. In some such cells, the cell is a human cell.

In another aspect, provided are methods of inserting a nucleic acid encoding a multidomain therapeutic protein comprising a delivery domain fused to a lysosomal alpha-glucosidase into a target genomic locus in a cell or a population of cells, comprising administering to the cell or the population of cells any of the above compositions, wherein the nuclease agent cleaves the nuclease target site in the target genomic locus, and the nucleic acid construct or the nucleic acid encoding the multidomain therapeutic protein is inserted into the target genomic locus. In some such methods, the percentage of unintended transcripts from the target genomic locus containing comprising the inserted nucleic acid construct or nucleic acid encoding the multidomain therapeutic protein is less than about 10%, less than about 9%, less than about 8%, less than about 7%, less than about 6%, less than about 5%, less than about 4%, less than about 3%, less than about 2%, or less than about 1%. In another aspect, provided are methods of expressing a multidomain therapeutic protein comprising a delivery domain fused to a lysosomal alpha-glucosidase in a cell or a population of cells, comprising administering to the cell or the population of cells any of the above compositions, wherein the coding sequence for the multidomain therapeutic protein is operably linked to a promoter in the nucleic acid construct and is expressed in the cell or population of cells. In another aspect, provided are methods of expressing a multidomain therapeutic protein comprising a delivery domain fused to a lysosomal alpha-glucosidase from a target genomic locus in a cell or a population of cells, comprising administering to the cell or the population of cells any of the above compositions, wherein the nuclease agent cleaves the nuclease target site in the target genomic locus, the nucleic acid construct or the coding sequence for the multidomain therapeutic protein is inserted into the target genomic locus to create a modified target genomic locus, and the multidomain therapeutic protein comprising the delivery domain fused to the lysosomal alpha-glucosidase is expressed from the modified target genomic locus. In some such methods, the percentage of unintended transcripts from the target genomic locus containing comprising the inserted nucleic acid construct or coding sequence for the multidomain therapeutic protein is less than about 10%, less than about 9%, less than about 8%, less than about 7%, less than about 6%, less than about 5%, less than about 4%, less than about 3%, less than about 2%, or less than about 1%.

In some such methods, the cell is a liver cell or a hepatocyte or the population of cells is a population of liver cells or hepatocytes. In some such methods, the cell is a human cell or the population of cells is a population of human cells. In some such methods, the cell is a neonatal cell or the population of cells is a population of neonatal cells. In some such methods, the neonatal cell or the population of neonatal cells is from a human neonatal subject within 24 weeks after birth, optionally wherein the neonatal cell or the population of neonatal cells is from a human neonatal subject within 12 weeks after birth, optionally wherein the neonatal cell or the population of neonatal cells is from a human neonatal subject within 8 weeks after birth, and optionally wherein the neonatal cell or the population of neonatal cells is from a human neonatal subject within 4 weeks after birth. In some such methods, the cell is not a neonatal cell or the population of cells is not a population of neonatal cells. In some such methods, the cell is in vitro or ex vivo or the population of cells is in vitro or ex vivo. In some such methods, the cell is in vivo in a subject or the population of cells is in vivo in a subject.

In another aspect, provided are methods of inserting a nucleic acid encoding a multidomain therapeutic protein comprising a delivery domain fused to a lysosomal alpha-glucosidase into a target genomic locus in a cell in a subject, comprising administering to the subject any of the above compositions, wherein the nuclease agent cleaves the nuclease target site in the target genomic locus, and the nucleic acid construct or the nucleic acid encoding the multidomain therapeutic protein is inserted into the target genomic locus. In some such methods, the percentage of unintended transcripts from the target genomic locus containing comprising the inserted nucleic acid construct or coding sequence for the multidomain therapeutic protein is less than about 10%, less than about 9%, less than about 8%, less than about 7%, less than about 6%, less than about 5%, less than about 4%, less than about 3%, less than about 2%, or less than about 1%. In another aspect, provided are methods of expressing a multidomain therapeutic protein comprising a delivery domain fused to a lysosomal alpha-glucosidase protein in a cell in a subject, comprising administering to the subject any of the above compositions, wherein the coding sequence for the multidomain therapeutic protein is operably linked to a promoter in the nucleic acid construct and is expressed in the cell. In another aspect, provided are methods of expressing a multidomain therapeutic protein comprising a delivery domain fused to a lysosomal alpha-glucosidase protein from a target genomic locus in a cell in a subject, comprising administering to the subject any of the above compositions, wherein the nuclease agent cleaves the nuclease target site in the target genomic locus, the nucleic acid construct or the coding sequence for the multidomain therapeutic protein is inserted into the target genomic locus to create a modified target genomic locus, and the multidomain therapeutic protein comprising the delivery domain fused to the lysosomal alpha-glucosidase is expressed from the modified target genomic locus. In some such methods, the percentage of unintended transcripts from the target genomic locus containing comprising the inserted nucleic acid construct or coding sequence for the multidomain therapeutic protein is less than about 10%, less than about 9%, less than about 8%, less than about 7%, less than about 6%, less than about 5%, less than about 4%, less than about 3%, less than about 2%, or less than about 1%.

In some such methods, the expressed multidomain therapeutic protein is delivered to and internalized by skeletal muscle and heart tissue in the subject or wherein the expressed multidomain therapeutic protein is delivered to and internalized by skeletal muscle, heart, and central nervous system tissue in the subject. In some such methods, the cell is a liver cell or a hepatocyte. In some such methods, the cell is a human cell. In some such methods, the cell is a neonatal cell. In some such methods, the neonatal subject is a human subject within 24 weeks after birth, optionally wherein the neonatal subject is a human subject within 12 weeks after birth, optionally wherein the neonatal subject is a human subject within 8 weeks after birth, and optionally wherein the neonatal subject is a human subject within 4 weeks after birth. In some such methods, the cell is not a neonatal cell.

In another aspect, provided are methods of treating a lysosomal alpha-glucosidase deficiency in a subject in need thereof, comprising administering to the subject any of the above compositions, wherein the coding sequence for the multidomain therapeutic protein is operably linked to a promoter in the nucleic acid construct and is expressed in the subject. In another aspect, provided are methods of treating a lysosomal alpha-glucosidase deficiency in a subject in need thereof, comprising administering to the subject any of the above compositions, wherein the nuclease agent cleaves the nuclease target site in the target genomic locus, the nucleic acid construct or the coding sequence for the multidomain therapeutic protein is inserted into the target genomic locus to create a modified target genomic locus, and the multidomain therapeutic protein comprising the delivery domain fused to the lysosomal alpha-glucosidase is expressed from the modified target genomic locus. In some such methods, the percentage of unintended transcripts from the target genomic locus containing comprising the inserted nucleic acid construct or coding sequence for the multidomain therapeutic protein is less than about 10%, less than about 9%, less than about 8%, less than about 7%, less than about 6%, less than about 5%, less than about 4%, less than about 3%, less than about 2%, or less than about 1%. In another aspect, provided are methods of reducing glycogen accumulation in a tissue in a subject in need thereof, comprising administering to the subject any of the above compositions, wherein the coding sequence for the multidomain therapeutic protein is operably linked to a promoter in the nucleic acid construct and is expressed in the subject and reduces glycogen accumulation in the tissue. In another aspect, provided are methods of reducing glycogen accumulation in a tissue in a subject in need thereof, comprising administering to the subject any of the above compositions, wherein the nuclease agent cleaves the nuclease target site in the target genomic locus, the nucleic acid construct or the coding sequence for the multidomain therapeutic protein is inserted into the target genomic locus to create a modified target genomic locus, and the multidomain therapeutic protein comprising the delivery domain fused to the lysosomal alpha-glucosidase is expressed from the modified target genomic locus and reduces glycogen accumulation in the tissue. In some such methods, the percentage of unintended transcripts from the target genomic locus containing comprising the inserted nucleic acid construct or coding sequence for the multidomain therapeutic protein is less than about 10%, less than about 9%, less than about 8%, less than about 7%, less than about 6%, less than about 5%, less than about 4%, less than about 3%, less than about 2%, or less than about 1%. In some such methods, the subject has Pompe disease. In another aspect, provided are methods of treating Pompe disease in a subject in need thereof, comprising administering to the subject any of the above compositions, wherein the coding sequence for the multidomain therapeutic protein is operably linked to a promoter in the nucleic acid construct and is expressed in the subject, thereby treating the Pompe disease. In another aspect, provided are methods of treating Pompe disease in a subject in need thereof, comprising administering to the subject any of the above compositions, wherein the nuclease agent cleaves the nuclease target site in the target genomic locus, the nucleic acid construct or the coding sequence for the multidomain therapeutic protein is inserted into the target genomic locus to create a modified target genomic locus, and the multidomain therapeutic protein comprising the delivery domain fused to the lysosomal alpha-glucosidase is expressed from the modified target genomic locus, thereby treating the Pompe disease. In some such methods, the percentage of unintended transcripts from the target genomic locus containing comprising the inserted nucleic acid construct or coding sequence for the multidomain therapeutic protein is less than about 10%, less than about 9%, less than about 8%, less than about 7%, less than about 6%, less than about 5%, less than about 4%, less than about 3%, less than about 2%, or less than about 1%.

In some such methods, the Pompe disease is infantile-onset Pompe disease. In some such methods, the Pompe disease is late-onset Pompe disease. In some such methods, the subject is a human subject. In some such methods, the subject is a neonatal subject, optionally wherein the neonatal subject is a human subject within 24 weeks after birth, within 12 weeks after birth, within 8 weeks after birth, or within 4 weeks after birth. In some such methods, the subject is not a neonatal subject. In some such methods, the method results in a therapeutically effective level of circulating multidomain therapeutic protein or lysosomal alpha-glucosidase in the subject. In some such methods, the method reduces glycogen accumulation in skeletal muscle, heart tissue, or central nervous system tissue in the subject, optionally wherein the method reduces glycogen accumulation in skeletal muscle, heart tissue, and central nervous system tissue in the subject, optionally wherein the method results in reduced glycogen levels in skeletal muscle, heart, and central nervous system tissue in the subject comparable to wild type levels at the same age, or wherein the method reduces glycogen accumulation in skeletal muscle or heart tissue in the subject, optionally wherein the method reduces glycogen accumulation in skeletal muscle and heart tissue in the subject, optionally wherein the method results in reduced glycogen levels in skeletal muscle and heart tissue in the subject comparable to wild type levels at the same age. In some such methods, the method improves muscle strength in the subject or prevents loss of muscle strength in the subject compared to a control subject. In some such methods, the method results in the subject having muscle strength comparable to wild type levels at the same age.

In another aspect, provided are methods of preventing or reducing the onset of a sign or symptom of Pompe disease in a subject in need thereof, comprising administering to the subject any of the above compositions, wherein the coding sequence for the multidomain therapeutic protein is operably linked to a promoter in the nucleic acid construct and is expressed in the subject, thereby preventing or reducing the onset of a sign or symptom of the Pompe disease in the subject. In another aspect, provided are methods of preventing or reducing the onset of a sign or symptom of Pompe disease in a subject in need thereof, comprising administering to the subject any of the above compositions, wherein the nuclease agent cleaves the nuclease target site, the nucleic acid construct or the coding sequence for the multidomain therapeutic protein is inserted into the target genomic locus to create a modified target genomic locus, and the multidomain therapeutic protein comprising the delivery domain fused to the lysosomal alpha-glucosidase is expressed from the modified target genomic locus, thereby preventing or reducing the onset of a sign or symptom of the Pompe disease in the subject. In some such methods, the percentage of unintended transcripts from the target genomic locus containing comprising the inserted nucleic acid construct or coding sequence for the multidomain therapeutic protein is less than about 10%, less than about 9%, less than about 8%, less than about 7%, less than about 6%, less than about 5%, less than about 4%, less than about 3%, less than about 2%, or less than about 1%.

In some such methods, the Pompe disease is infantile-onset Pompe disease. In some such methods, the Pompe disease is late-onset Pompe disease. In some such methods, the method results in a therapeutically effective level of circulating multidomain therapeutic protein or lysosomal alpha-glucosidase in the subject. In some such methods, the method prevents or reduces glycogen accumulation in skeletal muscle, heart, or central nervous system tissue in the subject. In some such methods, the method prevents or reduces glycogen accumulation in skeletal muscle, heart, and central nervous system tissue in the subject, or wherein the method prevents or reduces glycogen accumulation in skeletal muscle and heart tissue in the subject. In some such methods, the subject is a human subject. In some such methods, the subject is a neonatal subject. In some such methods, the neonatal subject is a human subject within 24 weeks after birth, optionally wherein the neonatal subject is a human subject within 12 weeks after birth, optionally wherein the neonatal subject is a human subject within 8 weeks after birth, and optionally wherein the neonatal subject is a human subject within 4 weeks after birth. In some such methods, the subject is not a neonatal subject.

In some such methods, the method results in increased expression of the multidomain therapeutic protein in the subject compared to a method comprising administering an episomal expression vector encoding the multidomain therapeutic protein to a control subject. In some such methods, the method results in increased serum levels of the multidomain therapeutic protein in the subject compared to a method comprising administering an episomal expression vector encoding the multidomain therapeutic protein to a control subject. In some such methods, the method results in serum levels of the multidomain therapeutic protein in the subject of at least about 1 μg/mL, at least about 2 μg/mL, at least about 3 μg/mL, at least about 4 μg/mL, at least about 5 μg/mL, at least about 6 μg/mL, at least about 7 μg/mL, at least about 8 μg/mL, at least about 9 μg/mL, or at least about 10 μg/mL. In some such methods, the method results in serum levels of the multidomain therapeutic protein in the subject of at least about 2 μg/mL or at least about 5 μg/mL. In some such methods, the method results in serum levels of the multidomain therapeutic protein in the subject of between about 2 μg/mL and about 30 μg/mL or between about 2 μg/mL and about 20 μg/mL. In some such methods, the method results in serum levels of the multidomain therapeutic protein in the subject of between about 5 μg/mL and about g/mL or between about 5 μg/mL and about 20 μg/mL. In some such methods, the method achieves lysosomal alpha-glucosidase activity levels of at least about 40% of normal, at least about 45% of normal, at least about 50%, at least about 60%, at least about 70%, at least about 80%, at least about 90%, or 100% of normal. In some such methods, (I) the subject has infantile-onset Pompe disease, and the method achieves lysosomal alpha-glucosidase expression or activity levels of at least about 1% or more than about 1% of normal; or (II) the subject has late-onset Pompe disease, and the method achieves lysosomal alpha-glucosidase expression or activity levels of at least about 40% of normal or more than about 40% of normal. In some such methods, the expression or activity of the multidomain therapeutic protein is at least 50% of the expression or activity of the multidomain therapeutic protein at a peak level of expression measured for the subject at 24 weeks after the administering. In some such methods, the expression or activity of the multidomain therapeutic protein is at least 50% of the expression or activity of the multidomain therapeutic protein at a peak level of expression measured for the subject at one year after the administering. In some such methods, the expression or activity of the multidomain therapeutic protein is at least 60% of the expression or activity of the multidomain therapeutic protein at a peak level of expression measured for the subject at 24 weeks after the administering. In some such methods, the expression or activity of the multidomain therapeutic protein is at least 50% of the expression or activity of the multidomain therapeutic protein at a peak level of expression measured for the subject at two years after the administering. In some such methods, the expression or activity of the multidomain therapeutic protein is at least 60% of the expression or activity of the multidomain therapeutic protein at a peak level of expression measured for the subject at 2 years after the administering. In some such methods, the expression or activity of the multidomain therapeutic protein is at least 60% of the expression or activity of the multidomain therapeutic protein at a peak level of expression measured for the subject at 24 weeks after the administering.

In some such methods, the method further comprises assessing preexisting AAV immunity in the subject prior to administering the nucleic acid construct to the subject. In some such methods, the preexisting AAV immunity is preexisting AAV8 immunity. In some such methods, assessing preexisting AAV immunity comprises assessing immunogenicity using a total antibody immune assay or a neutralizing antibody assay.

In some such methods, the nucleic acid construct is administered simultaneously with the nuclease agent or the one or more nucleic acids encoding the nuclease agent. In some such methods, the nucleic acid construct is not administered simultaneously with the nuclease agent or the one or more nucleic acids encoding the nuclease agent. In some such methods, the nucleic acid construct is administered prior to the nuclease agent or the one or more nucleic acids encoding the nuclease agent. In some such methods, the nucleic acid construct is administered after the nuclease agent or the one or more nucleic acids encoding the nuclease agent.

### BRIEF DESCRIPTION OF THE FIGURES

FIG. 1 shows amino acid sequences of various anti-human transferrin receptor scFv molecules in Vₖ-3×G₄S(SEQ ID NO: 616)-V_{H} format.

FIGS. 2A-2C show anti-human TFRC scFv antibody clones deliver GAA to the cerebrum of Tfrc^{hum} mice. Anti-human TfR:GAA molecules 69261, 69329, 12839, 12841, 12843 and 12845 (FIG. 2A) 69348, 12795, 12799, 12801, 12850 and 12798 (FIG. 2B); and 12802, 69340, 12847, 12848, 69307 and 69323 (FIG. 2C) were tested. Each lane=1 mouse. Delivery by HDD.

FIG. 3 shows a subset of anti-hTFRC antibodies (12798, 12850, 69323, 12841, 12843, 12845, 12847, 12848, 12799, 69307 and 12839) delivered mature GAA to the brain parenchyma in scfv:GAA format (delivery by HDD). Lane E corresponds to endothelium and Lane P corresponds to parenchyma. Ratio of affinity for mfTfR:human TfR are indicated below the image (mf refers to Macaca fascicularis monkey).

FIG. 4 shows anti-hTFRC antibodies (12799, 12843, 12847 and 12839) delivered mature GAA to the brain parenchyma in scfv:GAA format (AAV8 episomal liver depot gene therapy). Lane E corresponds to endothelium and Lane P corresponds to parenchyma.

FIG. 5 shows episomalAAV8 liver depot anti-hTFRC scfv:GAA antibodies delivered GAA protein to CNS (cerebellum, cerebrum, spinal cord), heart, and muscle (quadricep) in Gaa^{-/-}/Tfrc^{hum} mice.

FIG. 6 shows episomal AAV8 liver depot anti-hTFRC scfv:GAA antibodies (12839, 12843 and 12847) rescued glycogen storage in central nervous system (CNS) (cerebellum, cerebrum, spinal cord), heart, and muscle (quadricep) in Gaa^{-/-}/Tfrc^{hum} mice.

FIGS. 7A-7D show episomal AAV8 liver depot anti-hTFRC scfv:GAA antibodies (12847, 12843 and 12799) rescued glycogen storage in brain (brain thalamus (FIG. 7A), brain cerebral cortex (FIG. 7B), brain hippocampus CA1 (FIG. 7C)) and muscle (quadricep (FIG. 7D)) in Gaa^{-/-}/Tfrc^{hum} mice.

FIG. 8 shows albumin insertion of anti-hTFRC 12847scfv:GAA delivers mature GAA protein to CNS and muscle of Pompe model mice.

FIG. 9 shows albumin insertion of anti-hTFRC 12847scfv:GAA rescues glycogen storage in CNS and muscle of Pompe model mice. One Way ANOVA (* p<0.01; **p<0.001; ***p<0.0001).

FIG. 10 shows GAA activity in serum following Cas9-mediated insertion of AAV-delivered anti-TfR1:GAA or anti-CD63:GAA into the cynomolgus monkey albumin locus. Vehicle-only was used as a negative control. One unit of GAA activity is defined as the amount of enzyme that generates 1.0 μmol of 4-MU per min at pH 4.5 at 37° C. Error bars are SEM. N=1 for vehicle; N=2-4 for all others.

FIG. 11 shows albumin insertion of anti-hTFRC 12847scfv:GAA delivers mature GAA protein to CNS and muscle of cynomolgus monkeys. For the bar graphs, mature GAA was quantified by western blot of tissue lysates, and error bars are SD.

FIG. 12 shows the interaction of Mammarenavirus machupoense GP1 protein (PDB 3KAS), human ferritin (PDB 6GSR), Plasmodium vivax Sal-1 PvRBP2b protein (PDB 6D04), human HFE protein (PDB 1DE4), and human transferrin (PDB 1SUV) molecules superimposed on two TfR molecules in a symmetrical unit. For Mammarenavirus machupoense GP1 protein and human ferritin, only one copy in the symmetrical unit is shown to reduce complexity of the figure for clear view.

FIG. 13 depicts Hydrogen-Deuterium Exchange Mass Spectrometry (HDX) protections for the antibodies tested in HDX-MS experiments can be assigned to 5 regions in TfR (PDB 1SUV).

FIG. 14 illustrates TfR regions protected by REGN17513, a representation of antibodies that cause HDX protections in TfR apical domain that overlap with Mammarenavirus machupoense GP1 protein, human ferritin, and Plasmodium vivax PvRBP2b protein binding sites.

FIG. 15 illustrates TfR regions protected by REGN17510, a representation of antibodies with HDX protections in TfR apical domain that are not shared by other TfR binding partners shown in FIG. 15.

FIG. 16 illustrates TfR regions protected by REGN17515, a representation of antibodies with HDX protections in TfR apical domain that share binding sites with human ferritin and Plasmodium vivax Sal-1 PvRBP2b protein.

FIG. 17 illustrates TfR regions protected by REGN17514, a representation of antibodies with HDX protections in TfR protease-like domain and share binding sites with Plasmodium vivax Sal-1 PvRBP2b protein.

FIG. 18 illustrates TfR regions protected by REGN17508, a representation of antibodies with HDX protections in TfR protease-like domain. This region is not utilized by other TfR interacting molecules shown in FIG. 18.

FIGS. 19A and 19B show GAA enzymatic activity in the media after insertion of various anti-TfR:GAA insertion templates (CpG depleted and native) into the albumin locus of primary human hepatocytes after delivery by rAAV2.

FIG. 20A shows western blots showing that anti-human TfR antibody clones (0 CpG and native) deliver GAA to the brain (cerebrum) of 3-month-old Gaa^{-/-}/Tfrc^{hum} mice or Gaa^{-/-}/CD63^{hum} mice dosed intravenously with LNP-g666 (3 mg/kg) and various recombinant AAV8 anti-TfR:GAA or AAV8 anti-CD63:GAA insertion templates. Each lane=1 mouse.

FIG. 20B shows that albumin insertion of anti-hTfR:GAA rescues glycogen storage in cerebrum, quadriceps, diaphragm, and heart in Gaa^{-/-}/Tfrc^{hum} mice or Gaa^{-/-}/CD63^{hum} mice dosed intravenously with LNP-g666 (3 mg/kg) and various recombinant AAV8 anti-TfR:GAA or AAV8 anti-CD63:GAA insertion templates. Glycogen levels were measured at 3 weeks post-administration. Wt untreated mice were a positive control, and Gaa^{-/-} untreated mice were a negative control.

FIG. 21 shows levels of anti-CD63:GAA in the serum over a 10-month time course following administration of LNP-g666 (1 mg/kg) and a recombinant AAV8 anti-CD63:GAA insertion template (1.2e13 vg/kg) (“Insertion”) or following administration of episomal AAV encoding of anti-CD63:GAA (4e12 vg/kg) (“Episomal”) to adult Pompe disease model male and female mice (n=12; GAA^{-/-}; CD63 hu/hu).

FIG. 22 shows glycogen levels in the heart, quadricep, diaphragm, and spinal cord in Pompe disease model mice (GAA^{-/-}; CD63^{hu/hu}) at 10 months after administration of LNP-g666 and a recombinant AAV8 anti-CD63:GAA insertion template or at 10 months after administration of episomal AAV encoding anti-CD63:GAA to adult Pompe disease model male and female mice (n=12; GAA^{-/-}; CD63^{hu/hu}). Wild type GAA mice (GAA^{+/+}; CD63^{hu/hu}; n=4) and untreated Pompe disease model mice (n=4) were used as controls. The horizontal dotted line is the lower limit of detection of the assay.

FIGS. 23A-23B show levels of anti-CD63:GAA in the serum over a 15-month time course following administration of LNP-g666 and a recombinant AAV8 anti-CD63:GAA insertion template (n=10; male and female; “Insertion”) or following administration of episomal AAV encoding of anti-CD63:GAA (n=6; male and female; “Episomal”) to neonatal (P1) Pompe disease model mice (GAA^{-/-}; CD63^{hu/hu}). The horizontal dotted line is the lower limit of detection of the assay. The error bars in FIG. 23A are ±SD, and the error bars in FIG. 23B are ±SEM.

FIG. 24A shows glycogen levels in the heart, quadricep, gastrocnemius, and diaphragm in Pompe disease model mice (GAA^{-/-}; CD63^{hu/hu}) at 3 months after administration of LNP-g666 and a recombinant AAV8 anti-CD63:GAA insertion template (n=5; male and female, “I”) or at 3 months after administration of episomal AAV encoding anti-CD63:GAA (n=3; male and female, “E”) to neonatal (P1) mice. Untreated Pompe disease model mice were used as controls.

FIG. 24B shows glycogen levels in the heart, quadricep, gastrocnemius, diaphragm, cerebrum, and spinal cord in Pompe disease model mice (GAA^{-/-}; CD63^{hu/hu}) at 15 months after administration of LNP-g666 and a recombinant AAV8 anti-CD63:GAA insertion template (n=10; male and female, “I”) or at 15 months after administration of episomal AAV encoding anti-CD63:GAA (n=6; male and female, “E”) to neonatal (P1) mice. Untreated Pompe disease model mice (“U”) and wild type mice (“W”) were used as controls.

FIG. 25 shows grip strength in Pompe disease model mice (GAA^{-/-}; CD63^{hu/hu}) at 15 months after administration of LNP-g666 and a recombinant AAV8 anti-CD63:GAA insertion template (n=10; male and female, “P1 insertion AAV+LNP”) or at 15 months after administration of episomal AAV encoding anti-CD63:GAA (n=6; male and female, “P1 episomal AAV”) to neonatal (P1) mice. Wild type GAA mice (GAA^{+/+}; CD63^{hu/hu}; “Wild type”) and untreated Pompe disease model mice (“Untreated KO”) were used as controls.

FIG. 26 shows IFNα responses as measured by an IFNα ELISA in a primary human plasmacytoid DC-based assay. Various rAAV6 CpG-depleted anti-CD63:GAA templates were tested as compared to the first generation (non-CpG-depleted) anti-CD63:GAA template. rAAV6-GFP was used as a positive control, and a CpG-depleted (0 CpG) F9 template was used as a negative control.

FIG. 27 shows GAA enzymatic activity in the media after insertion of various anti-CD63:GAA and anti-TfR:GAA insertion templates into the albumin locus of primary human hepatocytes after delivery by rAAV2.

FIG. 28 shows GAA enzymatic activity in the media after insertion of various anti-CD63:GAA insertion templates into the albumin locus of primary human hepatocytes after delivery by rAAV6.

FIGS. 29A-29B show GAA serum expression in GAA^{-/-} mice following administration of LNP-g666 and various recombinant AAV8 anti-CD63:GAA insertion templates. Untreated KO and untreated WT mice were used as controls.

FIG. 30A shows GAA activity in serum measured using a fluorometric substrate assay in cynomolgus macaques that were administered recombinant AAV8 containing a CpG depleted anti-CD63:GAA template and LNP-g9860. Three different AAV8 doses were used (0.3e13 vg/kg, 1.5e13 vg/kg, and 5.6e13 vg/kg) with a 3 mg/kg LNP dose. N=1 in the vehicle control group, and N=3 in the dosed groups.

FIG. 30B shows expression of mature GAA in tissue lysates from cynomolgus macaques that were administered recombinant AAV8 containing a CpG depleted anti-CD63:GAA template and LNP-g9860. Three different AAV8 doses were used (0.3e13 vg/kg, 1.5e13 vg/kg, and 5.6e13 vg/kg) with a 3 mg/kg LNP dose. N=1 in the vehicle control group, and N=3 in the dosed groups. Tissues were collected at sacrifice (Day 89) and probed by western blot for presence of a 76 kDa lysosomal form of GAA.

FIG. 31 shows a schematic of LNP-g9860, which is a lipid nanoparticle containing Cas9 mRNA and sgRNA 9860 targeting human albumin (ALB) intron 1, and a recombinant AAV8 (rAAV8) capsid packaged with an anti-CD63:GAA insertion template.

FIG. 32 shows a schematic of targeting of GAA to the lysosome via fusion to anti-CD63 scFv.

FIG. 33 shows a schematic for CRISPR/Cas9-mediated insertion of an anti-CD63:GAA insertion template at the ALB locus. The human ALB locus is depicted, with the Cas9 cut site denoted with scissors. The splice acceptor site flanking the anti-CD63:GAA transgene in the insertion template is depicted. Following insertion and transcription driven by the endogenous ALB promoter, splicing between ALB exon 1 and the inserted anti-CD63:GAA DNA template occurs, diagrammed in dashed lines, to produce a hybrid ALB-anti-CD63: GAA mRNA. The ALB signal peptide promotes secretion of anti-CD63:GAA and is removed during protein maturation to yield anti-CD63:GAA in plasma.

FIG. 34 shows a schematic of LNP-g9860, which is a lipid nanoparticle containing Cas9 mRNA and sgRNA 9860 targeting human albumin (ALB) intron 1, and a recombinant AAV8 (rAAV8) capsid packaged with an anti-TfR:GAA insertion template.

FIG. 35 shows a schematic of targeting of GAA through multiple paths via fusion to anti-TfR scFv.

FIG. 36 shows a schematic for CRISPR/Cas9-mediated insertion of an anti-TfR:GAA insertion template at the ALB locus. The human ALB locus is depicted, with the Cas9 cut site denoted with scissors. The splice acceptor site flanking the anti-TfR:GAA transgene in the insertion template is depicted. Following insertion and transcription driven by the endogenous ALB promoter, splicing between ALB exon 1 and the inserted anti-TfR:GAA DNA template occurs, diagrammed in dashed lines, to produce a hybrid ALB-anti-TfR:GAA mRNA. The ALB signal peptide promotes secretion of anti-TfR:GAA and is removed during protein maturation to yield anti-TfR:GAA in plasma.

FIG. 37 shows ALB-anti-CD63: GAA transcripts identified in cynomolgus monkeys. During construct VVT1254-LNP-g9860-mediated gene insertion, the anti-CD63:GAA DNA template, supplied by construct VVT1254, is inserted into intron 1 of the ALB gene. The polyadenylation sequence following the anti-CD63:GAA transgene is labeled as pA. RNA sequencing analysis was performed to identify the splicing patterns in ALB-anti-CD63: GAA fusion transcripts produced in PHH incubated with construct VVT1254-LNP-g9860. The intended ALB-anti-CD63:GAA fusion transcripts have only 1 splicing event from ALB exon 1 to the splice acceptor site encoded within the inserted anti-CD63:GAA DNA template (intended transcript). Cryptic splice donor or acceptor sites that resulted in unintended transcripts are identified by arrow heads at the positions indicated. The unintended transcript, formed by splicing from nucleotide position 3078 to ALB exon 2, is shown as an example.

FIG. 38 shows GAA activity in supernatants from PXB human hepatocytes treated with LNP-g9860+AAVs encoding anti-CD63:GAA gene insertion templates with various modifications to cryptic splice sites and polyA sequences.

FIG. 39 shows GAA activity in supernatants from PXB human hepatocytes treated with LNP-g9860+AAVs encoding anti-TfR:GAA gene insertion templates with various modifications to cryptic splice sites and polyA sequences.

FIG. 40 shows GAA activity in supernatants from PXB human hepatocytes treated with LNP-g9860+AAVs encoding anti-CD63:GAA gene insertion templates with various modifications to cryptic splice sites and polyA sequences as compared to the original anti-CD63:GAA gene insertion template.

FIG. 41 shows GAA activity in supernatants from PXB human hepatocytes treated with LNP-g9860+AAVs encoding anti-TfR:GAA gene insertion templates with various modifications to cryptic splice sites and polyA sequences as compared to the original anti-TfR:GAA gene insertion template.

FIG. 42 shows the experimental setup for anti-TfR:GAA template validation in Tfrc^{hum/hum};Gaa^{-/-} mice.

FIG. 43 shows quantification of transgene anti-TfR:GAA DNA in liver nucleotide preps and quantification of anti-TfR:GAA mRNA expression in liver by Tagman using standard protocols.

FIG. 44 shows western blots showing that anti-human TfR antibody clones with mutations to remove cryptic splice sites and with different polyA sequences deliver GAA to the brain (cerebellum, cerebrum), muscle (quad), liver, and serum of 4-month-old Gaa^{-/-}/Tfrc^{hum} mice dosed intravenously with LNP-g666 (3 mg/kg) and various recombinant AAV8 anti-TfR:GAA insertion templates. Each lane=1 mouse.

FIGS. 45A-45B show that albumin insertion of anti-human TfR antibody clones with mutations to remove cryptic splice sites and with different polyA sequences rescues glycogen storage in brain (FIG. 45A) and muscle (FIG. 45B) in Gaa^{-/-}/Tfrc^{hum} mice dosed intravenously with LNP-g666 (3 mg/kg) and various recombinant AAV8 anti-TfR:GAA insertion templates. Glycogen levels were measured at 3 weeks post-administration. Wt untreated mice were a positive control, and Gaa^{-/-} untreated mice were a negative control.

FIG. 46 shows the experimental setup for anti-TfR:GAA template validation in albumin humanized mice.

FIGS. 47A-47C show western blots (FIG. 47A) and quantification of bands in the western blots (FIGS. 47B-47C) showing expression of anti-human TfR:GAA in serum and liver of 3-month-old humanized albumin mice dosed intravenously with LNP-g9860 (3 mg/kg) and various recombinant AAV8 anti-TfR:GAA insertion templates (3e12 vg/kg). Each lane in FIG. 47A=1 mouse.

FIG. 48 shows quantification of transgene anti-TfR:GAA DNA in liver nucleotide preps and quantification of anti-TfR:GAA mRNA expression in liver by Tagman using standard protocols.

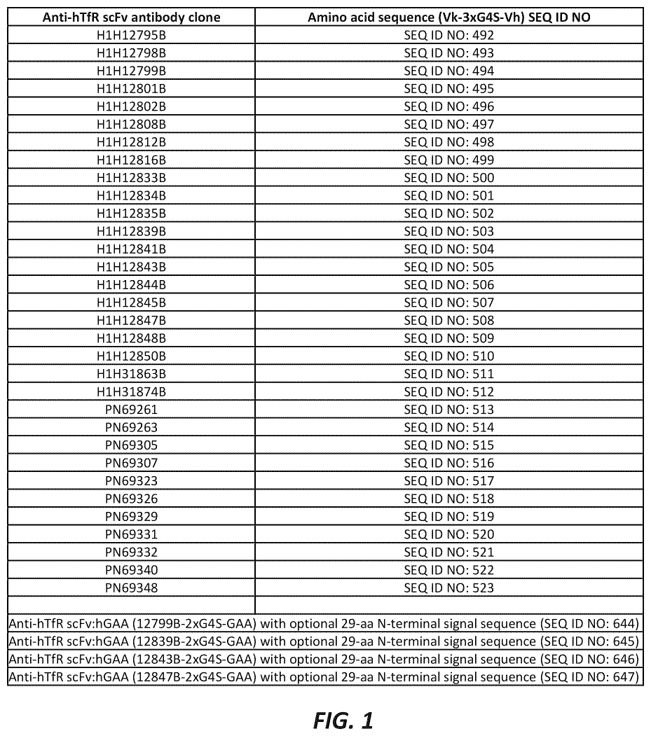

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “genomically integrated” refers to a nucleic acid that has been introduced into a cell such that the nucleotide sequence integrates into the genome of the cell. Any protocol may be used for the stable incorporation of a nucleic acid into the genome of a cell.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells in vitro, ex vivo, or in vivo. Numerous forms of viral vectors are known.

The term “isolated” with respect to cells, tissues (e.g., liver samples), proteins, and nucleic acids includes cells, tissues (e.g., liver samples), proteins, and nucleic acids that are relatively purified with respect to other bacterial, viral, cellular, or other components that may normally be present in situ, up to and including a substantially pure preparation of the cells, tissues (e.g., liver samples), proteins, and nucleic acids. The term “isolated” also includes cells, tissues (e.g., liver samples), proteins, and nucleic acids that have no naturally occurring counterpart, have been chemically synthesized and are thus substantially uncontaminated by other cells, tissues (e.g., liver samples), proteins, and nucleic acids, or has been separated or purified from most other components (e.g., cellular components) with which they are naturally accompanied (e.g., other cellular proteins, polynucleotides, or cellular components).

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a cell or animal. For example, an endogenous ALB sequence of a human refers to a native ALB sequence that naturally occurs at the ALB locus in the human.

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” (i.e., “codon optimized” sequences) takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a polypeptide of interest can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Res. 28(1):292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. For example, an “ALB locus” may refer to the specific location of an ALB gene, ALB DNA sequence, albumin-encoding sequence, or ALB position on a chromosome of the genome of an organism that has been identified as to where such a sequence resides. An “ALB locus” may comprise a regulatory element of an ALB gene, including, for example, an enhancer, a promoter, 5′ and/or 3′ untranslated region (UTR), or a combination thereof.

The term “gene” refers to DNA sequences in a chromosome that may contain, if naturally present, at least one coding and at least one non-coding region. The DNA sequence in a chromosome that codes for a product (e.g., but not limited to, an RNA product and/or a polypeptide product) can include the coding region interrupted with non-coding introns and sequence located adjacent to the coding region on both the 5′ and 3′ ends such that the gene corresponds to the full-length mRNA (including the 5′ and 3′ untranslated sequences). Additionally, other non-coding sequences including regulatory sequences (e.g., but not limited to, promoters, enhancers, and transcription factor binding sites), polyadenylation signals, internal ribosome entry sites, silencers, insulating sequence, and matrix attachment regions may be present in a gene. These sequences may be close to the coding region of the gene (e.g., but not limited to, within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a mouse cell, a rat cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The methods and compositions provided herein employ a variety of different components. Some components throughout the description can have active variants and fragments. The term “functional” refers to the innate ability of a protein or nucleic acid (or a fragment or variant thereof) to exhibit a biological activity or function. The biological functions of functional fragments or variants may be the same or may in fact be changed (e.g., with respect to their specificity or selectivity or efficacy) in comparison to the original molecule, but with retention of the molecule's basic biological function.

The term “variant” refers to a nucleotide sequence differing from the sequence most prevalent in a population (e.g., by one nucleotide) or a protein sequence different from the sequence most prevalent in a population (e.g., by one amino acid).

The term “fragment,” when referring to a protein, means a protein that is shorter or has fewer amino acids than the full-length protein. The term “fragment,” when referring to a nucleic acid, means a nucleic acid that is shorter or has fewer nucleotides than the full-length nucleic acid. A fragment can be, for example, when referring to a protein fragment, an N-terminal fragment (i.e., removal of a portion of the C-terminal end of the protein), a C-terminal fragment (i.e., removal of a portion of the N-terminal end of the protein), or an internal fragment (i.e., removal of a portion of each of the N-terminal and C-terminal ends of the protein). A fragment can be, for example, when referring to a nucleic acid fragment, a 5′ fragment (i.e., removal of a portion of the 3′ end of the nucleic acid), a 3′ fragment (i.e., removal of a portion of the 5′ end of the nucleic acid), or an internal fragment (i.e., removal of a portion each of the 5′ and 3′ ends of the nucleic acid).

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

TABLE 1
Amino Acid Categorizations.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “in vitro” includes artificial environments and to processes or reactions that occur within an artificial environment (e.g., a test tube or an isolated cell or cell line). The term “in vivo” includes natural environments (e.g., a cell or organism or body) and to processes or reactions that occur within a natural environment. The term “ex vivo” includes cells that have been removed from the body of an individual and processes or reactions that occur within such cells.

The term “antibody,” as used herein, includes immunoglobulin molecules comprising four polypeptide chains, two heavy (H) chains and two light (L) chains inter-connected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL or VK) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. The term “high affinity” antibody refers to those antibodies having a binding affinity to their target of at least 10⁻⁹ M, at least 1010 M; at least 10⁻¹² M; or at least 10⁻¹² M, as measured by surface plasmon resonance, e.g., BIACORE™ or solution-affinity ELISA. The term “antibody” may encompass any type of antibody, such as, e.g., monoclonal or polyclonal. Moreover, the antibody may be or any origin, such as, e.g., mammalian or non-mammalian. In one embodiment, the antibody may be mammalian or avian. In a further embodiment, the antibody may be or human origin and may further be a human monoclonal antibody.

The phrase “bispecific antibody” includes an antibody capable of selectively binding two or more epitopes. Bispecific antibodies generally comprise two different heavy chains, with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

The phrase “heavy chain,” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen (e.g., recognizing the antigen with a KD in the micromolar, nanomolar, or picomolar range), that is capable of expressing and secreting from a cell, and that comprises at least one CDR.

The phrase “light chain” includes an immunoglobulin light chain constant region sequence from any organism, and unless otherwise specified includes human kappa and lambda light chains. Light chain variable (VL) domains typically include three light chain CDRs and four framework (FR) regions, unless otherwise specified. Generally, a full-length light chain includes, from amino terminus to carboxyl terminus, a VL domain that includes FR1-CDR1-FR2-CDR2-FR3-CDR3-FR4, and a light chain constant domain. Light chains that can be used herein include, for example, those that do not selectively bind either the first or second antigen selectively bound by the antigen-binding protein. Suitable light chains include those that can be identified by screening for the most commonly employed light chains in existing antibody libraries (wet libraries or in silico), where the light chains do not substantially interfere with the affinity and/or selectivity of the antigen-binding domains of the antigen-binding proteins. Suitable light chains include those that can bind one or both epitopes that are bound by the antigen-binding regions of the antigen-binding protein.

The phrase “variable domain” includes an amino acid sequence of an immunoglobulin light or heavy chain (modified as desired) that comprises the following amino acid regions, in sequence from N-terminal to C-terminal (unless otherwise indicated): FR1, CDR1, FR2, CDR2, FR3, CDR3, FR4. A “variable domain” includes an amino acid sequence capable of folding into a canonical domain (VH or VL) having a dual beta sheet structure wherein the beta sheets are connected by a disulfide bond between a residue of a first beta sheet and a second beta sheet.

The phrase “complementarity determining region,” or the term “CDR,” includes an amino acid sequence encoded by a nucleic acid sequence of an organism's immunoglobulin genes that normally (i.e., in a wild type animal) appears between two framework regions in a variable region of a light or a heavy chain of an immunoglobulin molecule (e.g., an antibody or a T cell receptor). A CDR can be encoded by, for example, a germline sequence or a rearranged or unrearranged sequence, and, for example, by a naive or a mature B cell or a T cell. In some circumstances (e.g., for a CDR3), CDRs can be encoded by two or more sequences (e.g., germline sequences) that are not contiguous (e.g., in an unrearranged nucleic acid sequence) but are contiguous in a B cell nucleic acid sequence, for example, as the result of splicing or connecting the sequences (e.g., V-D-J recombination to form a heavy chain CDR3).

The term “antibody fragment” refers to one or more fragments of an antibody that retain the ability to specifically bind to an antigen. Examples of binding fragments encompassed within the term “antibody fragment” include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 241:544-546), which consists of a VH domain, (vi) an isolated CDR, and (vii) an scFv, which consists of the two domains of the Fv fragment, VL and VH, joined by a synthetic linker to form a single protein chain in which the VL and VH regions pair to form monovalent molecules. Other forms of single chain antibodies, such as diabodies are also encompassed under the term “antibody” (see e.g., Holliger et al. (1993) Proc. Natl. Acad. Sci. U.S.A. 90:6444-6448; Poljak et al. (1994) Structure 2:1121-1123).

The phrase “Fc-containing protein” includes antibodies, bispecific antibodies, immunoadhesins, and other binding proteins that comprise at least a functional portion of an immunoglobulin CH2 and CH3 region. A “functional portion” refers to a CH2 and CH3 region that can bind a Fc receptor (e.g., an FcγR; or an FcRn, i.e., a neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional.

Fc-containing proteins can comprise modifications in immunoglobulin domains, including where the modifications affect one or more effector function of the binding protein (e.g., modifications that affect FcγR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

For example, and not by way of limitation, the binding protein is an Fc-containing protein and exhibits enhanced serum half-life (as compared with the same Fc-containing protein without the recited modification(s)) and have a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at 428 and/or 433 (e.g., L/R/SI/P/Q or K) and/or 434 (e.g., H/F or Y); or a modification at 250 and/or 428; or a modification at 307 or 308 (e.g., 308F, V308F), and 434. In another example, the modification can comprise a 428L (e.g., M428L) and 434S (e.g., N434S) modification; a 428L, 2591 (e.g., V259I), and a 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); a 307 and/or 308 modification (e.g., 308F or 308P).

The term “antigen-binding protein,” as used herein, refers to a polypeptide or protein (one or more polypeptides complexed in a functional unit) that specifically recognizes an epitope on an antigen, such as a cell-specific antigen and/or a target antigen provided herein. An antigen-binding protein may be multi-specific. The term “multi-specific” with reference to an antigen-binding protein means that the protein recognizes different epitopes, either on the same antigen or on different antigens. A multi-specific antigen-binding protein provided herein can be a single multifunctional polypeptide, or it can be a multimeric complex of two or more polypeptides that are covalently or non-covalently associated with one another. The term “antigen-binding protein” includes antibodies or fragments thereof provided herein that may be linked to or co-expressed with another functional molecule, for example, another peptide or protein. For example, an antibody or fragment thereof can be functionally linked (e.g., by chemical coupling, genetic fusion, non-covalent association or otherwise) to one or more other molecular entities, such as a protein or fragment thereof to produce a bispecific or a multi-specific antigen-binding molecule with a second binding specificity.

As used herein, the term “epitope” refers to the portion of the antigen which is recognized by the multi-specific antigen-binding polypeptide. A single antigen (such as an antigenic polypeptide) may have more than one epitope. Epitopes may be defined as structural or functional. Functional epitopes are generally a subset of structural epitopes and are defined as those residues that directly contribute to the affinity of the interaction between the antigen-binding polypeptide and the antigen. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes formed from contiguous amino acids are typically retained on exposure to denaturing solvents, whereas epitopes formed by tertiary folding are typically lost on treatment with denaturing solvents.

The term “domain” refers to any part of a protein or polypeptide having a particular function or structure. Preferably, domains provided herein bind to cell-specific or target antigens. Cell-specific antigen- or target antigen-binding domains, and the like, as used herein, include any naturally occurring, enzymatically obtainable, synthetic, or genetically engineered polypeptide or glycoprotein that specifically binds an antigen.

The term “half-body” or “half-antibody,” which are used interchangeably, refers to half of an antibody, which essentially contains one heavy chain and one light chain. Antibody heavy chains can form dimers, thus the heavy chain of one half-body can associate with heavy chain associated with a different molecule (e.g., another half-body) or another Fc-containing polypeptide. Two slightly different Fc-domains may “heterodimerize” as in the formation of bispecific antibodies or other heterodimers, -trimers, -tetramers, and the like. See Vincent and Murini (2012) Biotechnol. J. 7(12):1444-1450; and Shimamoto et al. (2012) MAbs 4(5):586-91. In one embodiment, the half-body variable domain specifically recognizes the internalization effector and the half body Fc-domain dimerizes with an Fc-fusion protein that comprises a replacement enzyme (e.g., a peptibody).

The term “single-chain variable fragment” or “scFv” includes a single chain fusion polypeptide containing an immunoglobulin heavy chain variable region (VH) and an immunoglobulin light chain variable region (VL). In some embodiments, the VH and VL are connect by a linker sequence of 10 to 25 amino acids. ScFv polypeptides may also include other amino acid sequences, such as CL or CH1 regions. ScFv molecules can be manufactured by phage display or made by directly subcloning the heavy and light chains from a hybridoma or B-cell. See Ahmad et al. (2012) Clin. Dev. Immunol. 2012:980250, herein incorporated by reference in its entirety for all purposes.

As used herein, the term “neonatal” in the context of humans covers human subjects up to or under the age of 1 year (52 weeks), preferably up to or under the age of 24 weeks, more preferably up to or under the age of 12 weeks, more preferably up to or under the age of 8 weeks, and even more preferably up to or under the age of 4 weeks. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals. As used herein, a “neonatal cell” is a cell of a neonatal subject, and a population of neonatal cells is a population of cells of a neonatal subject.

As used herein, a “control” as in a control sample or a control subject is a comparator for a measurement, e.g., a diagnostic measurement of a sign or symptom of a disease. In certain embodiments, a control can be a subject sample from the same subject an earlier time point, e.g., before a treatment intervention. In certain embodiments, a control can be a measurement from a normal subject, i.e., a subject not having the disease of the treated subject, to provide a normal control, e.g., an enzyme concentration or activity in a subject sample. In certain embodiments, a normal control can be a population control, i.e., the average of subjects in the general population. In certain embodiments, a control can be an untreated subject with the same disease. In certain embodiments, a control can be a subject treated with a different therapy, e.g., the standard of care. In certain embodiments, a control can be a subject or a population of subjects from a natural history study of subjects with the disease of the subject being compared. In certain embodiments, the control is matched for certain factors to the subject being tested, e.g., age, gender. In certain embodiments, a control may be a control level for a particular lab, e.g., a clinical lab. Selection of an appropriate control is within the ability of those of skill in the art.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range. For example, 5-10 nucleotides is understood as 5, 6, 7, 8, 9, or 10 nucleotides, whereas 5-10% is understood to contain 5% and all possible values through 10%.

At least 17 nucleotides of a 20 nucleotide sequence is understood to include 17, 18, 19, or 20 nucleotides of the sequence provided, thereby providing an upper limit even if one is not specifically provided as it would be clearly understood. Similarly, up to 3 nucleotides would be understood to encompass 0, 1, 2, or 3 nucleotides, providing a lower limit even if one is not specifically provided. When “at least,” “up to,” or other similar language modifies a number, it can be understood to modify each number in the series.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, “detecting an analyte” and the like is understood as performing an assay in which the analyte can be detected, if present, wherein the analyte is present in an amount above the level of detection of the assay.

As used herein, “loss of function” is understood as an activity not being present, e.g., an enzyme activity not being present, for any reason. In certain embodiments, the absence of activity may be due to the absence of a protein having a function, e.g., protein is not transcribed or translated, protein is translated but not stable or not transported appropriately, either intracellularly or systemically. In certain embodiments, the absence of activity may be due to the presence of a mutation, e.g., point mutation, truncation, abnormal splicing, such that a protein is present, but not functional. A loss of function can be a partial or complete loss of function. In certain embodiments, various degrees of loss of function may be known that result in various conditions, severity of disease, or age of onset. As used herein, a loss of function is preferably not a transient loss of function, e.g., due to a stress response or other response that results in a temporary loss of a functional protein. Therapeutic interventions to correct for a loss of function of a protein may include compensation for the loss of function with the protein that is deficient, or with proteins that compensate for the loss of function, but that have a different sequence or structure than the protein for which the function is lost. It is understood that a loss of function of one protein may be compensated for by providing or altering the activity of another protein in the same biological pathway. In certain embodiments, the protein to compensate for the loss of function includes one or more of a truncation, mutation, or non-native sequence to direct trafficking of the protein, either intracellularly or systemically, to overcome the loss of function of the protein. The therapeutic intervention may or may not correct the loss of function of the protein in all cell types or tissues. The therapeutic intervention may include expression of the protein to compensate for a loss of function at a site remote from where the protein lacking function is typically expressed, e.g., where the deficiency results in dysfunction of a cell or organ. The therapeutic intervention may include expression of the protein in the liver to compensate for a loss of function at a site remote from the liver. A number of genetic mutations have been linked with specific loss of function mutations, in both humans and other species.

As used herein, “enzyme deficiency” is understood as an insufficient level of an enzyme activity due to a loss of function of the protein. An enzyme deficiency can be partial or total, and may result in differences in time of onset or severity of signs or symptoms of the enzyme deficiency depending on the level and site of the loss of function. As used herein, enzyme deficiency is preferably not a transient enzyme deficiency due to stress or other factors. A number of genetic mutations have been linked with enzyme deficiencies, in both humans and other species. In certain embodiments, enzyme deficiencies result in inborn errors of metabolism. In certain embodiments, enzyme deficiencies result in lysosomal storage diseases. In certain embodiments, enzyme deficiencies result in galactosemia. In certain embodiments, enzyme deficiencies result in bleeding disorders.

As used herein, it is understood that when the maximum amount of a value is represented by 100% (e.g., 100% inhibition or 100% encapsulation) that the value is limited by the method of detection. For example, 100% inhibition is understood as inhibition to a level below the level of detection of the assay, and 100% encapsulation is understood as no material intended for encapsulation can be detected outside the vesicles.

Unless otherwise apparent from the context, the term “about” encompasses values ±5% of a stated value. In certain embodiments, the term “about” is understood to encompass tolerated variation or error within the art, e.g., 2 standard deviations from the mean, or the sensitivity of the method used to take a measurement, or a percent of a value as tolerated in the art, e.g., with age. When “about” is present before the first value of a series, it can be understood to modify each value in the series.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p<0.05.

In the event of a conflict between a sequence in the application and an indicated accession number or position in an accession number, the sequence in the application predominates.

### DETAILED DESCRIPTION

### I. Overview

Compositions and methods for inserting a nucleic acid encoding a multidomain therapeutic protein (e.g., GAA fusion protein) into a target genomic locus in a cell, a population of cells, or a subject (e.g., a neonatal cell, a population of neonatal cells, or a neonatal subject) or for expressing a nucleic acid encoding a multidomain therapeutic protein (e.g., GAA fusion protein) from a target genomic locus in a cell, a population of cells, or a subject (e.g., a neonatal cell, a population of neonatal cells, or a neonatal subject) are also provided. Compositions and methods for treating GAA deficiency, reducing glycogen accumulation in a tissue, treating Pompe disease, or preventing or reducing the onset of a sign or symptom of Pompe disease in a subject (e.g., a neonatal subject) are provided. Also provided are cells or populations of cells (e.g., neonatal cells or populations of neonatal cells) comprising a nucleic acid construct comprising a coding sequence for a multidomain therapeutic protein (e.g., GAA fusion protein) inserted into a target genomic locus.

Also provided herein are nucleic acid constructs and compositions (e.g., episomal expression vectors) for expression of a multidomain therapeutic protein (e.g., GAA fusion protein). Also provided herein are nucleic acid constructs and compositions that allow insertion of a multidomain therapeutic protein (e.g., GAA fusion protein) coding sequence into a target genomic locus such as an endogenous ALB locus and/or expression of the multidomain therapeutic protein (e.g., GAA fusion protein) coding sequence. The nucleic acid constructs and compositions can be used in methods of integrating or inserting a multidomain therapeutic protein (e.g., GAA fusion protein) nucleic acid into a target genomic locus in a cell or a population of cells or a subject, methods of expressing a multidomain therapeutic protein (e.g., GAA fusion protein) in a cell or a population of cells or a subject, methods of reducing glycogen accumulation in a cell or a population of cells or a subject, methods of treating Pompe disease or GAA deficiency in a subject, and method of preventing or reducing the onset of a sign or symptom of Pompe disease in a subject, including neonatal cells and subjects.

Also provided are compositions or combinations or kits comprising a nucleic acid construct comprising a coding sequence for the multidomain therapeutic protein in combination with a nuclease agent or one or more nucleic acids encoding the nuclease agent, wherein the nuclease agent targets a nuclease target site in a target genomic locus. As used herein, the term “in combination with” means that additional component(s) may be administered prior to, concurrent with, or after the administration of the nucleic acid construct. The different components of the combination can be formulated into a single composition, e.g., for simultaneous delivery, or formulated separately into two or more compositions (e.g., a kit including each component, for example, wherein the further agent is in a separate formulation).

More specifically, described herein in some embodiments is a therapeutic product based on the CRISPR/Cas9 gene editing technology and optionally contained in a lipid nanoparticle (LNP) delivery system, associated with a multidomain therapeutic protein (e.g., GAA fusion protein) DNA gene insertion template optionally contained in a recombinant adeno-associated virus serotype 8 (rAAV8). The CRISPR/Cas9 component has been designed to target and cut the double stranded DNA at a target gene locus (e.g., a safe harbor locus such as an ALB gene locus in hepatocytes), allowing for the multidomain therapeutic protein (e.g., GAA fusion protein) DNA template to be inserted in the genome at the target genomic locus. Transgene insertion provides a functional multidomain therapeutic protein (e.g., GAA fusion protein) gene, encoding the missing or defective genomic GAA in Pompe disease patients.

Some of the multidomain therapeutic protein (e.g., GAA fusion protein) coding sequences in the constructs disclosed herein are optimized for expression as compared to native GAA coding sequence. For example, the coding sequences in the constructs disclosed herein may include one or more modifications such as codon optimization (e.g., to human codons), depletion of CpG dinucleotides, mutation of cryptic splice sites, or any combination thereof. Other multidomain therapeutic protein coding sequences in the constructs disclosed herein comprise native GAA coding sequences.

In some embodiments, to minimize mis-splicing in the multidomain therapeutic protein nucleic acid constructs, we applied a two-pronged approach: (1) identifying cryptic splice donors functionally via RNA sequencing (RNA-Seq), rather than prediction based on consensus sequences, and introducing synonymous mutations that disrupt the critical “GU” nucleotide pair that forms the core of the splice donor sequence, and (2) adding additional elements which have the net effect of increasing the time from when the polyA is transcribed to when RNA polymerase reaches the next splice acceptor site. We introduced either concatenated polyA signals (e.g., bovine growth hormone (BGH) and SV40), MAZ elements which cause polymerase pausing, or additional stuffer sequence to increase the time between when RNA polymerase transcribes the polyA to the time when it transcribes the next splice acceptor. The SV40 polyA is bidirectional, but the polyadenylation in the “late” orientation is more efficient than the polyadenylation in the “early” orientation. In some embodiments, to concatenate SV40 “late” polyA with BGH polyA, we mutated the transcription terminator sequences that are present in the “early” inverse orientation of SV40, thus making this version of the SV40 polyA unidirectional rather than bidirectional. Thus, if our DNA insertion templates are inserted into the genome in the non-functional “reverse” orientation, transcription should proceed straight through the entire locus (e.g., albumin locus) and the non-functional insertion should be spliced out along with the first intron, as there are no transcription terminator sequences present in the “reverse” orientation.

II. Multidomain Therapeutic Proteins and Compositions for Inserting Nucleic Acid Constructs Encoding and/or for Expressing Multidomain Therapeutic Proteins in Cells

Multidomain therapeutic proteins comprising a TfR-binding delivery domain or a CD63-binding delivery domain fused to a lysosomal alpha-glucosidase (GAA) polypeptide and nucleic acid constructs and compositions that allow insertion of a multidomain therapeutic protein coding sequence into a target genomic locus such as an endogenous ALB locus and/or expression of the multidomain therapeutic protein coding sequence are provided. The multidomain therapeutic proteins and nucleic acid constructs and compositions can be administered to cells, populations of cells, or subjects and can be used in methods of integration of a multidomain therapeutic protein nucleic acid into a target genomic locus, methods of expression of a multidomain therapeutic protein in a cell or population of cells or a subject, methods of reducing glycogen accumulation in a cell or a population of cells or a tissue in a subject, methods of treating Pompe disease or GAA deficiency in a subject, and methods of preventing or reducing the onset of a sign or symptom of Pompe disease or GAA deficiency in a subject.

Provided herein are multidomain therapeutic proteins comprising a TfR-binding delivery domain or a CD63-binding delivery domain fused to a lysosomal alpha-glucosidase (GAA) polypeptide. The multidomain therapeutic proteins and compositions can be used in methods of introducing a multidomain therapeutic protein into a cell or a population of cells or a subject, methods of treating Pompe disease or GAA deficiency in a subject, and methods of preventing or reducing the onset of a sign or symptom of Pompe disease or GAA deficiency in a subject

Provided herein are nucleic acid constructs and compositions that allow insertion of a multidomain therapeutic protein coding sequence into a target genomic locus such as an endogenous albumin (ALB) locus and/or expression of the multidomain therapeutic protein coding sequence. Also provided herein are nucleic acid constructs and compositions (e.g., episomal expression vectors) for expression of a multidomain therapeutic protein. The nucleic acid constructs and compositions can be used in methods of introducing a nucleic acid construct comprising a multidomain therapeutic protein coding sequence into a cell or a population of cells or a subject, methods of integration of a multidomain therapeutic protein nucleic acid into a target genomic locus, methods of expression of a multidomain therapeutic protein in a cell, methods of treating Pompe disease or GAA deficiency in a subject, and methods of preventing or reducing the onset of a sign or symptom of Pompe disease or GAA deficiency in a subject. Also provided are nuclease agents (e.g., targeting an endogenous ALB locus) or nucleic acids encoding nuclease agents to facilitate integration of the nucleic acid constructs into a target genomic locus such as an endogenous ALB locus.

### A. Multidomain Therapeutic Proteins and Nucleic Acid Constructs Encoding a Multidomain Therapeutic Protein

The compositions and methods described herein include the use of multidomain therapeutic proteins comprising a lysosomal alpha-glucosidase (GAA) polypeptide (GAA or a biologically active portion thereof, to provide GAA enzyme replacement activity) linked to or fused to a TfR-binding delivery domain or a CD63-binding delivery domain. The compositions and methods described herein also include the use of a nucleic acid construct that comprises a coding sequence for a multidomain therapeutic protein. The compositions and methods described herein can also include the use of a nucleic acid construct that comprises a multidomain therapeutic protein coding sequence or a reverse complement of the multidomain therapeutic protein coding sequence. Such nucleic acid constructs can be for expression of the multidomain therapeutic protein in a cell. Such nucleic acid constructs can be for insertion into a target genomic locus or into a cleavage site created by a nuclease agent or CRISPR/Cas system as disclosed elsewhere herein. The term cleavage site includes a DNA sequence at which a nick or double-strand break is created by a nuclease agent (e.g., a Cas9 protein complexed with a guide RNA). In some embodiments, a double-stranded break is created by a Cas9 protein complexed with a guide RNA, e.g., a Spy Cas9 protein complexed with a Spy Cas9 guide RNA.

The length of the nucleic acid constructs disclosed herein can vary. The construct can be, for example, from about 1 kb to about 5 kb, such as from about 1 kb to about 4.5 kb or about 1 kb to about 4 kb. An exemplary nucleic acid construct is between about 1 kb to about 5 kb in length or between about 1 kb to about 4 kb in length. Alternatively, a nucleic acid construct can be between about 1 kb to about 1.5 kb, about 1.5 kb to about 2 kb, about 2 kb to about 2.5 kb, about 2.5 kb to about 3 kb, about 3 kb to about 3.5 kb, about 3.5 kb to about 4 kb, about 4 kb to about 4.5 kb, or about 4.5 kb to about 5 kb in length. Alternatively, a nucleic acid construct can be, for example, no more than 5 kb, no more than 4.5 kb, no more than 4 kb, no more than 3.5 kb, no more than 3 kb, or no more than 2.5 kb in length.

The constructs can comprise deoxyribonucleic acid (DNA) or ribonucleic acid (RNA), can be single-stranded, double-stranded, or partially single-stranded and partially double-stranded, and can be introduced into a host cell in linear or circular (e.g., minicircle) form. See, e.g., US 2010/0047805, US 2011/0281361, and US 2011/0207221, each of which is herein incorporated by reference in their entirety for all purposes. If introduced in linear form, the ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in their entirety for all purposes. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. A construct can be introduced into a cell as part of a vector molecule having additional sequences such as, for example, replication origins, promoters, and genes encoding antibiotic resistance. A construct may omit viral elements. Moreover, constructs can be introduced as a naked nucleic acid, can be introduced as a nucleic acid complexed with an agent such as a liposome or poloxamer, or can be delivered by viruses (e.g., adenovirus, adeno-associated virus (AAV), herpesvirus, retrovirus, or lentivirus).

The constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Some constructs may be inserted so that their expression is driven by the endogenous promoter at the insertion site (e.g., the endogenous ALB promoter when the construct is integrated into the host cell's ALB locus). Such constructs may not comprise a promoter that drives the expression of the multidomain therapeutic protein. For example, the expression of the multidomain therapeutic protein can be driven by a promoter of the host cell (e.g., the endogenous ALB promoter when the transgene is integrated into a host cell's ALB locus). In such cases, the construct may lack control elements (e.g., promoter and/or enhancer) that drive its expression (e.g., a promoterless construct). In other cases, the construct may comprise a promoter and/or enhancer, for example, a constitutive promoter or an inducible or tissue-specific (e.g., liver- or platelet-specific) promoter that drives expression of the multidomain therapeutic protein in an episome or upon integration. For example, the construct may be a construct for expression (e.g., an episomal construct) but not for insertion. In some embodiments, the construct is not for insertion. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. For example, the promoter may be a CMV promoter or a truncated CMV promoter. In another example, the promoter may be an EF1a promoter. Non-limiting exemplary inducible promoters include those inducible by heat shock, light, chemicals, peptides, metals, steroids, antibiotics, or alcohol. The inducible promoter may be one that has a low basal (non-induced) expression level, such as the Tet-On® promoter (Clontech). Although not required for expression, the constructs may comprise transcriptional or translational regulatory sequences such as promoters, enhancers, insulators, internal ribosome entry sites, additional sequences encoding peptides, and/or polyadenylation signals. The construct may comprise a sequence encoding a multidomain therapeutic protein downstream of and operably linked to a signal sequence encoding a signal peptide. In some examples, the nucleic acid construct works in homology-independent insertion of a nucleic acid that encodes a multidomain therapeutic protein. Such nucleic acid constructs can work, for example, in non-dividing cells (e.g., cells in which non-homologous end joining (NHEJ), not homologous recombination (HR), is the primary mechanism by which double-stranded DNA breaks are repaired) or dividing cells (e.g., actively dividing cells). Such constructs can be, for example, homology-independent donor constructs. In preferred embodiments, promoters and other regulatory sequences are appropriate for use in humans, e.g., recognized by regulatory factors in human cells, e.g., in human liver cells, and acceptable to regulatory authorities for use in humans. Examples of liver-specific promoters include TTR promoters, such as human or mouse TTR promoters. In one example, the construct may comprise a TTR promoter, such as a mouse TTR promoter or a human TTR promoter (e.g., the coding sequence for the multidomain therapeutic protein is operably linked to the TTR promoter). In one example, the construct may comprise a SERPINA1 enhancer, such as a mouse SERPINA1 enhancer or a human SERPINA1 enhancer (e.g., the coding sequence for the multidomain therapeutic protein is operably linked to the SERPINA1 enhancer). In one example, the construct may comprise a TTR promoter and a SERPINA1 enhancer, such as a human SERPINA1 enhancer and a mouse TTR promoter (e.g., the coding sequence for the multidomain therapeutic protein is operably linked to the SERPINA1 enhancer and the TTR promoter).

The constructs disclosed herein can be modified to include or exclude any suitable structural feature as needed for any particular use and/or that confers one or more desired function. For example, some constructs disclosed herein do not comprise a homology arm. Some constructs disclosed herein are capable of insertion into a target genomic locus or a cut site in a target DNA sequence for a nuclease agent (e.g., capable of insertion into a safe harbor gene, such as an ALB locus) by non-homologous end joining. For example, such constructs can be inserted into a blunt end double-strand break following cleavage with a nuclease agent (e.g., CRISPR/Cas system, e.g., a SpyCas9 CRISPR/Cas system) as disclosed herein. In a specific example, the construct can be delivered via AAV and can be capable of insertion by non-homologous end joining (e.g., the construct does not comprise a homology arm).

In a particular example, the construct can be inserted via homology-independent targeted integration. For example, the multidomain therapeutic protein coding sequence in the construct can be flanked on each side by a target site for a nuclease agent (e.g., the same target site as in the target DNA sequence for targeted insertion (e.g., in a safe harbor gene), and the same nuclease agent being used to cleave the target DNA sequence for targeted insertion). The nuclease agent can then cleave the target sites flanking the multidomain therapeutic protein. In a specific example, the construct is delivered AAV-mediated delivery, and cleavage of the target sites flanking the multidomain therapeutic protein coding sequence can remove the inverted terminal repeats (ITRs) of the AAV. In some instances, the target DNA sequence for targeted insertion (e.g., target DNA sequence in a safe harbor locus such as a gRNA target sequence including the flanking protospacer adjacent motif) is no longer present if the multidomain therapeutic protein coding sequence is inserted into the cut site or target DNA sequence in the correct orientation but it is reformed if the multidomain therapeutic protein coding sequence is inserted into the cut site or target DNA sequence in the opposite orientation. This can help ensure that the multidomain therapeutic protein coding sequence) is inserted in the correct orientation for expression.

The constructs disclosed herein can comprise a polyadenylation sequence or polyadenylation tail sequence (e.g., downstream or 3′ of a multidomain therapeutic protein coding sequence). Methods of designing a suitable polyadenylation tail sequence are well-known. The polyadenylation tail sequence can be encoded, for example, as a “poly-A” stretch downstream of the multidomain therapeutic protein coding sequence. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes. The term polyadenylation signal sequence refers to any sequence that directs termination of transcription and addition of a poly-A tail to the mRNA transcript. In eukaryotes, transcription terminators are recognized by protein factors, and termination is followed by polyadenylation, a process of adding a poly(A) tail to the mRNA transcripts in presence of the poly(A) polymerase. The mammalian poly(A) signal typically consists of a core sequence, about 45 nucleotides long, that may be flanked by diverse auxiliary sequences that serve to enhance cleavage and polyadenylation efficiency. The core sequence consists of a highly conserved upstream element (AATAAA or AAUAAA) in the mRNA, referred to as a poly A recognition motif or poly A recognition sequence), recognized by cleavage and polyadenylation-specificity factor (CPSF), and a poorly defined downstream region (rich in Us or Gs and Us), bound by cleavage stimulation factor (CstF). Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. In one example, the polyadenylation signal is a simian virus 40 (SV40) late polyadenylation signal. For example, the polyadenylation signal can comprise, consist essentially of, or consist of SEQ ID NO: 615, 169, or 161. For example, the polyadenylation signal can comprise, consist essentially of, or consist of SEQ ID NO: 169 or 161. For example, the polyadenylation signal can comprise, consist essentially of, or consist of SEQ ID NO: 169. For example, the polyadenylation signal can comprise, consist essentially of, or consist of SEQ ID NO: 615. In another example, the polyadenylation signal is a bovine growth hormone (BGH) polyadenylation signal or a CpG depleted BGH polyadenylation signal. For example, the polyadenylation signal can comprise, consist essentially of, or consist of SEQ ID NO: 162.

In one example, the polyadenylation signal can comprise a BGH polyadenylation signal. For example, the BGH polyadenylation signal can comprise, consist essentially of, or consist of SEQ ID NO: 751. In another example, the polyadenylation signal can comprise an SV40 polyadenylation signal. For example, the SV40 polyadenylation signal can be a unidirectional SV40 late polyadenylation signal. For example, the transcription terminator sequences that are present in the “early” inverse orientation of SV40 can be mutated (e.g., by mutating the reverse strand AAUAAA sequences to AAUCAA). The SV40 polyA is bidirectional, but the polyadenylation in the “late” orientation is more efficient than the polyadenylation in the “early” orientation. For example, the unidirectional SV40 late polyadenylation signal can comprise, consist essentially of, or consist of SEQ ID NO: 752. In another example, a synthetic polyadenylation signal can be used. For example, the synthetic polyadenylation signal can comprise, consist essentially of, or consist of SEQ ID NO: 753. In another example, two or more polyadenylation signals can be used in combination. For example, the polyadenylation signal can comprise a combination of a BGH polyadenylation signal and an SV40 polyadenylation signal (e.g., an SV40 late polyadenylation signal, such as a unidirectional SV40 late polyadenylation signal). For example, the polyadenylation signal can comprise a combination of a BGH polyadenylation signal and a unidirectional SV40 late polyadenylation signal. For example, the BGH polyadenylation signal can comprise, consist essentially of, or consist of SEQ ID NO: 751, and the unidirectional SV40 late polyadenylation signal can comprise, consist essentially of, or consist of SEQ ID NO: 752. In a specific example, the BGH polyadenylation signal can be upstream (5′) of the SV40 polyadenylation signal (e.g., unidirectional SV40 late polyadenylation signal). For example, the combined polyadenylation signal can comprise the sequence set forth in SEQ ID NO: 795. In another example, the polyadenylation signal can comprise a combination of a BGH polyadenylation signal and a synthetic polyadenylation signal. For example, the BGH polyadenylation signal can comprise, consist essentially of, or consist of SEQ ID NO: 751, and the synthetic polyadenylation signal can comprise, consist essentially of, or consist of SEQ ID NO: 753. In some embodiments, the nucleic acid construct is a unidirectional construct.

In some embodiments, a stuffer sequence can be used to increase the time between when RNA polymerase transcribes the polyA to the time when it transcribes the next splice acceptor. For example, the stuffer sequence can be used between two different polyadenylation signals (e.g., between a BGH polyadenylation signal and a synthetic polyadenylation signal. For example, the stuffer sequence can comprise, consist essentially of, or consist of SEQ ID NO: 754.

In some embodiments, MAZ elements that cause polymerase pausing are used in combination with a polyadenylation signal (e.g., a BGH polyadenylation signal or an SV40 polyadenylation signal). For example, one or more (e.g., at least 1, at least 2, at least 3, at least 4, or about 1 to about 4, about 2 to about 4, about 3 to about 4, or 1, 2, 3, or 4) MAZ elements can be used in combination with a polyadenylation signal. For example, the MAZ element can comprise, consist essentially of, or consist of SEQ ID NO: 755.

In some embodiments, unidirectional SV40 late polyadenylation signals are used. The SV40 polyA is bidirectional, but the polyadenylation in the “late” orientation is more efficient than the polyadenylation in the “early” orientation. The unidirectional SV40 late polyadenylation signals described herein are positioned in the “late” orientation, with the polyadenylation signals present in the “early” orientation mutated or inactivated. In some embodiments, each instance of the sequence AATAAA in the reverse strand is mutated in the unidirectional SV40 late polyadenylation signal. For example, the two conserved AATAAA poly(A) signals present in the SV40 “early” poly(A) to AATCAA. In some embodiments, the unidirectional SV40 late polyadenylation signal is at least 95%, at least 96%, at least 97%, at least 98%, or at least 99% identical to the sequence set forth in SEQ ID NO: 752. In some embodiments, the unidirectional SV40 late polyadenylation signal comprises, consists essentially of, or consists of the sequence set forth in SEQ ID NO: 752.

The unidirectional SV40 late polyadenylation signals can be used in combination with (e.g., in tandem with) one or more additional polyadenylation signals. Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. For example, the unidirectional SV40 late polyadenylation signals can be used in combination with (e.g., in tandem with) a bovine growth hormone (BGH) polyadenylation signal, optionally wherein the BGH polyadenylation signal is upstream of (5′ of) the unidirectional SV40 late polyadenylation signal. In some embodiments, the BGH polyadenylation signal is at least 95%, at least 96%, at least 97%, at least 98%, or at least 99% identical to the sequence set forth in SEQ ID NO: 751. In some embodiments, the BGH polyadenylation signal comprises, consists essentially of, or consists of the sequence set forth in SEQ ID NO: 751. In some embodiments, the combination of the BGH polyadenylation signal and the unidirectional SV40 late polyadenylation signal is at least 95%, at least 96%, at least 97%, at least 98%, or at least 99% identical to the sequence set forth in SEQ ID NO: 795. In some embodiments, the combination of the BGH polyadenylation signal and the unidirectional SV40 late polyadenylation signal comprises, consists essentially of, or consists of the sequence set forth in SEQ ID NO: 795.

In some embodiments, a stuffer sequence can be used to increase the time between when RNA polymerase transcribes the polyA to the time when it transcribes the next splice acceptor. For example, the stuffer sequence can be used between two different polyadenylation signals (e.g., between a BGH polyadenylation signal and a synthetic polyadenylation signal. For example, the stuffer sequence can comprise, consist essentially of, or consist of SEQ ID NO: 754.

In some embodiments, MAZ elements that cause polymerase pausing are used in combination with a polyadenylation signal (e.g., a BGH polyadenylation signal or an SV40 polyadenylation signal). For example, one or more (e.g., at least 1, at least 2, at least 3, at least 4, or about 1 to about 4, about 2 to about 4, about 3 to about 4, or 1, 2, 3, or 4) MAZ elements can be used in combination with a polyadenylation signal. For example, the MAZ element can comprise, consist essentially of, or consist of SEQ ID NO: 755.

The constructs disclosed herein may also comprise splice acceptor sites (e.g., operably linked to the multidomain therapeutic protein coding sequence, such as upstream or 5′ of the multidomain therapeutic protein coding sequence). The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a splice acceptor from a gene encoding the polypeptide of interest (e.g., a GAA splice acceptor). For example, such a splice acceptor can be derived from the human GAA gene. Alternatively, such a splice acceptor can be derived from the mouse GAA gene. Additional suitable splice acceptor sites useful in eukaryotes, including artificial splice acceptors, are well-known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes. In a specific example, the splice acceptor is a mouse Alb exon 2 splice acceptor. In a specific example, the splice acceptor can comprise, consist essentially of, or consist of SEQ ID NO: 163.

In some examples, the nucleic acid constructs disclosed herein can be bidirectional constructs, which are described in more detail below. In some examples, the nucleic acid constructs disclosed herein can be unidirectional constructs, which are described in more detail below. Likewise, in some examples, the nucleic acid constructs disclosed herein can be in a vector (e.g., viral vector, such as AAV, or rAAV8) and/or a lipid nanoparticle as described in more detail elsewhere herein.

### (1) Multidomain Therapeutic Proteins

A multidomain therapeutic protein as described herein includes a lysosomal alpha-glucosidase polypeptide (GAA or a biologically active portion thereof, to provide GAA enzyme replacement activity) linked to or fused to a TfR-binding delivery domain or a CD63-binding delivery domain. TfR-binding domains, CD63-binding delivery domains, and GAA polypeptides are described in more detail below. Examples of multidomain therapeutic proteins can be found in WO 2013/138400, WO 2017/007796, WO 2017/190079, WO 2017/100467, WO 2018/226861, WO 2019/157224, and WO 2019/222663, each of which is herein incorporated by reference in its entirety for all purposes. For example, the multidomain therapeutic proteins described herein can comprise a TfR-binding delivery domain linked to or fused to a GAA polypeptide. The TfR-binding domain provides binding to the internalization factor TfR. The multidomain therapeutic protein produced by the liver is targeted the muscle and CNS by targeting TfR, which is expressed in muscle and on brain endothelial cells. Transcytosis of TfR in these cells enables blood-brain-barrier crossing. For example, the multidomain therapeutic proteins described herein can comprise a CD63-binding delivery domain linked to or fused to a GAA polypeptide. The CD63-binding domain provides binding to the internalization factor CD63. The multidomain therapeutic protein is targeted to the muscle by targeting CD63, which is a rapidly internalizing protein highly expressed in the muscle. In some multidomain therapeutic proteins, the delivery domain is covalently linked to the GAA. The covalent linkage may be any type of covalent bond (i.e., any bond that involved sharing of electrons). In some cases, the covalent bond is a peptide bond between two amino acids, such that the GAA and the delivery domain in whole or in part form a continuous polypeptide chain, as in a fusion protein. In some cases, the GAA portion and the delivery domain portion are directly linked. In other cases, a linker, such as a peptide linker, is used to tether the two portions. Any suitable linker can be used. See Chen et al., “Fusion protein linkers: property, design and functionality,” 65(10) Adv Drug Deliv Rev. 1357-69 (2013). In some cases, a cleavable linker is used. For example, a cathepsin cleavable linker can be inserted between the delivery domain and the GAA to facilitate removal of the delivery domain in the lysosome. In another example, the linker can comprise an amino acid sequence, e.g., about 10 amino acids in length, for example, 1, 2, 3, 4, 5, 6, 7, 8, 8, or 10 repeats of Gly4Ser (SEQ ID NO: 537). In one example, the linker comprises, consists essentially of, or consists of three such repeats (SEQ ID NO: 616). For example, the coding sequence for the linker can comprise, consist essentially of, or consist of any one of SEQ ID NOS: 618-622 and 747. In another example, the linker comprises, consists essentially of, or consists of two such repeats (SEQ ID NO: 617). For example, the coding sequence for the linker can comprise, consist essentially of, or consist of any one of SEQ ID NOS: 623-629. In another example, the linker comprises, consists essentially of, or consists of one such repeat (SEQ ID NO: 537). For example, the coding sequence for the linker can comprise, consist essentially of, or consist of SEQ ID NO: 630 or 748. In another example, a rigid linker can be used such as a 2λH4 linker. In one example, the linker comprises, consists essentially of, or consists of AEAAAKEAAAKEAAAKEAAAKALEAEAAAKEAAAKEAAAKEAAAKA (SEQ ID NO: 842). For example, the coding sequence for the linker can comprise, consist essentially of, or consist of SEQ ID NO: 841.

In a particular multidomain therapeutic protein, the GAA (e.g., N-terminus) is covalently linked to the C-terminus of the heavy chain of an anti-TfR or anti-CD63 antibody or to the C-terminus of the light chain (i.e., the multidomain therapeutic protein is in the format of anti-TfR:GAA or anti-CD63:GAA from N-terminus to C-terminus). In another particular multidomain therapeutic protein, the GAA is covalently linked to the N-terminus of the heavy chain of an anti-TfR or anti-CD63 antibody or to the N-terminus of the light chain (i.e., the multidomain therapeutic protein is in the format of GAA:anti-TfR or GAA:anti-CD63 from N-terminus to C-terminus). In another particular embodiment, the GAA (e.g., N-terminus) is linked to the C-terminus of an anti-TfR or anti-CD63 scFv domain (i.e., the multidomain therapeutic protein is in the format of anti-TfR-scFv:GAA or anti-CD63-scFv:GAA, such as anti-TfR-scFv(V_{L}V_{H}):GAA or anti-CD63-scFv(V_{L}V_{H}):GAA, from N-terminus to C-terminus). In another particular embodiment, the GAA (e.g., N-terminus) is linked to the C-terminus of an anti-TfR or anti-CD63 Fab heavy chain (i.e., the multi domain therapeutic protein is in the format of anti-TfR-Fab(LightHeavy):GAA or anti-CD63-Fab(LightHeavy):GAA from N-terminus to C-terminus). In another particular embodiment, the GAA (e.g., N-terminus) is linked to the C-terminus of an anti-TfR or anti-CD63 Fab light chain (i.e., the multi domain therapeutic protein is in the format of anti-TfR-Fab(HeavyLight):GAA or anti-CD63-Fab(HeavyLight):GAA from N-terminus to C-terminus).

### (a) Lysosomal Alpha-Glucosidase (GAA)

Lysosomal alpha-glucosidase (GAA; also known as acid alpha-glucosidase, acid alpha-glucosidase preproprotein, acid maltase, aglucosidase alfa, alpha-1,4-glucosidase, amyloglucosidase, glucoamylase, LYAG) is encoded by GAA. This enzyme is active in lysosomes, where it breaks down glycogen into glucose.

The human GAA gene (NCBI GeneID 2548) encodes a 952 amino acid protein. In the lysosome, human GAA is sequentially processed by proteases to polypeptides of 76-, 19.4-, and 3.9-kDa that remain associated. Further cleavage between R(200) and A(204) inefficiently converts the 76-kDa polypeptide to the mature 70-kDa form with an additional 10.4-kDa polypeptide. GAA maturation increases its affinity for glycogen by 7-10 fold. A signal peptide is encoded by amino acids 1-27, a propeptide encoded by amino acids 28-69, lysosomal alpha-glucosidase after removal of the signal peptide and propeptide is encoded by amino acids 70-952, the 76 kDa lysosomal alpha-glucosidase is encoded by amino acids 123-952, and the 70 kDa lysosomal alpha-glucosidase is encoded by amino acids 204-952.

The GAA expressed from the compositions and methods disclosed herein can be any wild type or variant GAA. In one example, the GAA is a human GAA protein. Human GAA is assigned UniProt reference number P10253. An exemplary amino acid sequence for human GAA is assigned NCBI Accession No. NP_000143.2 and is set forth in SEQ ID NO: 724. An exemplary human GAA mRNA (cDNA) sequence is assigned NCBI Accession No. NM_000152.5 and is set forth in SEQ ID NO: 725. An exemplary human GAA coding sequence is assigned CCDS ID CCDS32760.1 and is set forth in SEQ ID NO: 726. An exemplary mature human GAA amino acid sequence (i.e., the human GAA sequence after removal of the signal peptide and propeptide) starting at amino acid 70 (i.e., GAA 70-952) is set forth in SEQ ID NO: 727. An exemplary coding sequence for GAA 70-952 is set forth in SEQ ID NO: 728.

In some examples, the GAA (e.g., human GAA) is a wild type GAA (e.g., wild type human GAA) sequence or a fragment thereof. For example, the GAA can be a fragment comprising the mature GAA amino acid sequence (i.e., the GAA sequence after removal of the signal peptide and propeptide), a fragment comprising the 77 kDa form of GAA, or a fragment comprising the 70 kDa form of GAA. In a specific example, the GAA can comprise SEQ ID NO: 727 or can be at least 90%, at least 91%, at least 92%, at least 93%, at least 94%, at least 95%, at least 96%, at least 97%, at least 98%, at least 99%, or at least 99.5% identical to SEQ ID NO: 727. In another specific example, the GAA can consist essentially of SEQ ID NO: 727. In another specific example, the GAA can consist of SEQ ID NO: 727.

The GAA coding sequences in the constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a GAA coding sequence in a construct disclosed herein has one or more cryptic splice sites mutated or removed. In some embodiments, the nucleotide at position 1095 (or the corresponding position when the GAA coding sequence is aligned with SEQ ID NO: 750) is a “G.” In some embodiments, the nucleotide at position 1098 (or the corresponding position when the GAA coding sequence is aligned with SEQ ID NO: 750) is a “C.” In some embodiments, the nucleotide at position 2343 (or the corresponding position when the GAA coding sequence is aligned with SEQ ID NO: 750) is a “G.” In some embodiments, the nucleotide at position 1095 (or the corresponding position when the GAA coding sequence is aligned with SEQ ID NO: 750) is a “G,” the nucleotide at position 1098 (or the corresponding position when the GAA coding sequence is aligned with SEQ ID NO: 750) is a “C,” and the nucleotide at position 2343 (or the corresponding position when the GAA coding sequence is aligned with SEQ ID NO: 750) is a “G.” In another example, a GAA coding sequence in a construct disclosed herein has all identified cryptic splice sites mutated or removed. In another example, a GAA coding sequence in a construct disclosed herein has one or more CpG dinucleotides removed (i.e., is CpG depleted). In another example, a GAA coding sequence in a construct disclosed herein has all CpG dinucleotides removed (i.e., is fully CpG depleted). In another example, a GAA coding sequence in a construct disclosed herein is codon optimized (e.g., codon optimized for expression in a human or mammal). In a specific example, a GAA coding sequence in a construct disclosed herein has one or more CpG dinucleotides removed (i.e., is CpG depleted) and has one or more cryptic splice sites mutated or removed. In another specific example, a GAA coding sequence in a construct disclosed herein has all CpG dinucleotides removed and has one or more or all identified cryptic splice sites mutated or removed. In another specific example, a GAA coding sequence in a construct disclosed herein has one or more CpG dinucleotides removed (i.e., is CpG depleted) and is codon optimized (e.g., codon optimized for expression in a human or mammal). In another specific example, a GAA coding sequence in a construct disclosed herein has all CpG dinucleotides removed (i.e., is fully CpG depleted) and is codon optimized (e.g., codon optimized for expression in a human or mammal).

Various codon optimized GAA coding sequences are provided. The GAA coding sequence can be, for example, CpG-depleted (e.g., fully CpG depleted) and/or codon optimized (e.g., CpG depleted (e.g., fully CpG-depleted) and codon optimized). In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 750, 749, and 649. In another example, the GAA coding sequence is (or comprises a sequence) at least 95%, at least 96%, at least 97%, at least 98%, at least 99%, at least 99.5%, or 100% identical to any one of SEQ ID NOS: 750, 749, and 649. In another example, the GAA coding sequence is (or comprises a sequence) at least 99%, at least 99.5%, or 100% identical to any one of SEQ ID NOS: 750, 749, and 649. In another example, the GAA coding sequence comprises the sequence set forth in any one of SEQ ID NOS: 750, 749, and 649. In another example, the GAA coding sequence consists essentially of the sequence set forth in any one of SEQ ID NOS: 750, 749, and 649. In another example, the GAA coding sequence consists of the sequence set forth in any one of SEQ ID NOS: 750, 749, and 649. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 727 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 727 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 727 (and, e.g., retaining the activity of native GAA). Optionally, the GAA coding sequence in the above examples encodes a GAA protein comprising the sequence set forth in SEQ ID NO: 727. Optionally, the GAA coding sequence in the above examples encodes a GAA protein consisting essentially of the sequence set forth in SEQ ID NO: 727. Optionally, the GAA coding sequence in the above examples encodes a GAA protein consisting of the sequence set forth in SEQ ID NO: 727. In some embodiments, the nucleotide at position 1095 (or the corresponding position when the GAA coding sequence is aligned with SEQ ID NO: 750) is a “G.” In some embodiments, the nucleotide at position 1098 (or the corresponding position when the GAA coding sequence is aligned with SEQ ID NO: 750) is a “C.” In some embodiments, the nucleotide at position 2343 (or the corresponding position when the GAA coding sequence is aligned with SEQ ID NO: 750) is a “G.” In some embodiments, the nucleotide at position 1095 (or the corresponding position when the GAA coding sequence is aligned with SEQ ID NO: 750) is a “G,” the nucleotide at position 1098 (or the corresponding position when the GAA coding sequence is aligned with SEQ ID NO: 750) is a “C,” and the nucleotide at position 2343 (or the corresponding position when the GAA coding sequence is aligned with SEQ ID NO: 750) is a “G.”

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 750. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 750 and encodes a GAA protein (or a GAA protein comprising a sequence) at least 99%, at least 99.5%, or 100% identical to SEQ ID NO: 727. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 750 and encodes a GAA protein comprising the sequence set forth in SEQ ID NO: 727. In another example, the GAA coding sequence is (or comprises a sequence) at least 95%, at least 96%, at least 97%, at least 98%, at least 99%, at least 99.5%, or 100% identical to SEQ ID NO: 750. In another example, the GAA coding sequence is (or comprises a sequence) at least 95%, at least 96%, at least 97%, at least 98%, at least 99%, at least 99.5%, or 100% identical to SEQ ID NO: 750 and encodes a GAA protein (or a GAA protein comprising a sequence) at least 99%, at least 99.5%, or 100% identical to SEQ ID NO: 727. In another example, the GAA coding sequence is (or comprises a sequence) at least 95%, at least 96%, at least 97%, at least 98%, at least 99%, at least 99.5%, or 100% identical to SEQ ID NO: 750 and encodes a GAA protein comprising the sequence set forth in SEQ ID NO: 727. In another example, the GAA coding sequence is (or comprises a sequence) at least 99%, at least 99.5%, or 100% identical to SEQ ID NO: 750. In another example, the GAA coding sequence is (or comprises a sequence) at least 99%, at least 99.5%, or 100% identical to SEQ ID NO: 750 and encodes a GAA protein (or a GAA protein comprising a sequence) at least 99%, at least 99.5%, or 100% identical to SEQ ID NO: 727. In another example, the GAA coding sequence is (or comprises a sequence) at least 99%, at least 99.5%, or 100% identical to SEQ ID NO: 750 and encodes a GAA protein comprising the sequence set forth in SEQ ID NO: 727. In another example, the GAA coding sequence comprises the sequence set forth in SEQ ID NO: 750. In another example, the GAA coding sequence consists essentially of the sequence set forth in SEQ ID NO: 750. In another example, the GAA coding sequence consists of the sequence set forth in SEQ ID NO: 750. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 727 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 727 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 727 (and, e.g., retaining the activity of native GAA). Optionally, the GAA coding sequence in the above examples encodes a GAA protein comprising the sequence set forth in SEQ ID NO: 727. Optionally, the GAA coding sequence in the above examples encodes a GAA protein consisting essentially of the sequence set forth in SEQ ID NO: 727. Optionally, the GAA coding sequence in the above examples encodes a GAA protein consisting of the sequence set forth in SEQ ID NO: 727. In some embodiments, the nucleotide at position 1095 (or the corresponding position when the GAA coding sequence is aligned with SEQ ID NO: 750) is a “G.” In some embodiments, the nucleotide at position 1098 (or the corresponding position when the GAA coding sequence is aligned with SEQ ID NO: 750) is a “C.” In some embodiments, the nucleotide at position 2343 (or the corresponding position when the GAA coding sequence is aligned with SEQ ID NO: 750) is a “G.” In some embodiments, the nucleotide at position 1095 (or the corresponding position when the GAA coding sequence is aligned with SEQ ID NO: 750) is a “G,” the nucleotide at position 1098 (or the corresponding position when the GAA coding sequence is aligned with SEQ ID NO: 750) is a “C,” and the nucleotide at position 2343 (or the corresponding position when the GAA coding sequence is aligned with SEQ ID NO: 750) is a “G.”

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 749. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 749 and encodes a GAA protein (or a GAA protein comprising a sequence) at least 99%, at least 99.5%, or 100% identical to SEQ ID NO: 727. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 749 and encodes a GAA protein comprising the sequence set forth in SEQ ID NO: 727. In another example, the GAA coding sequence is (or comprises a sequence) at least 95%, at least 96%, at least 97%, at least 98%, at least 99%, at least 99.5%, or 100% identical to SEQ ID NO: 749. In another example, the GAA coding sequence is (or comprises a sequence) at least 95%, at least 96%, at least 97%, at least 98%, at least 99%, at least 99.5%, or 100% identical to SEQ ID NO: 749 and encodes a GAA protein (or a GAA protein comprising a sequence) at least 99%, at least 99.5%, or 100% identical to SEQ ID NO: 727. In another example, the GAA coding sequence is (or comprises a sequence) at least 95%, at least 96%, at least 97%, at least 98%, at least 99%, at least 99.5%, or 100% identical to SEQ ID NO: 749 and encodes a GAA protein comprising the sequence set forth in SEQ ID NO: 727. In another example, the GAA coding sequence is (or comprises a sequence) at least 99%, at least 99.5%, or 100% identical to SEQ ID NO: 749. In another example, the GAA coding sequence is (or comprises a sequence) at least 99%, at least 99.5%, or 100% identical to SEQ ID NO: 749 and encodes a GAA protein (or a GAA protein comprising a sequence) at least 99%, at least 99.5%, or 100% identical to SEQ ID NO: 727. In another example, the GAA coding sequence is (or comprises a sequence) at least 99%, at least 99.5%, or 100% identical to SEQ ID NO: 749 and encodes a GAA protein comprising the sequence set forth in SEQ ID NO: 727. In another example, the GAA coding sequence comprises the sequence set forth in SEQ ID NO: 749. In another example, the GAA coding sequence consists essentially of the sequence set forth in SEQ ID NO: 749. In another example, the GAA coding sequence consists of the sequence set forth in SEQ ID NO: 749. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 727 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 727 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 727 (and, e.g., retaining the activity of native GAA). Optionally, the GAA coding sequence in the above examples encodes a GAA protein comprising the sequence set forth in SEQ ID NO: 727. Optionally, the GAA coding sequence in the above examples encodes a GAA protein consisting essentially of the sequence set forth in SEQ ID NO: 727. Optionally, the GAA coding sequence in the above examples encodes a GAA protein consisting of the sequence set forth in SEQ ID NO: 727. In some embodiments, the nucleotide at position 1095 (or the corresponding position when the GAA coding sequence is aligned with SEQ ID NO: 750) is a “G.” In some embodiments, the nucleotide at position 1098 (or the corresponding position when the GAA coding sequence is aligned with SEQ ID NO: 750) is a “C.” In some embodiments, the nucleotide at position 2343 (or the corresponding position when the GAA coding sequence is aligned with SEQ ID NO: 750) is a “G.” In some embodiments, the nucleotide at position 1095 (or the corresponding position when the GAA coding sequence is aligned with SEQ ID NO: 750) is a “G,” the nucleotide at position 1098 (or the corresponding position when the GAA coding sequence is aligned with SEQ ID NO: 750) is a “C,” and the nucleotide at position 2343 (or the corresponding position when the GAA coding sequence is aligned with SEQ ID NO: 750) is a “G.”

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 649. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 649 and encodes a GAA protein (or a GAA protein comprising a sequence) at least 99%, at least 99.5%, or 100% identical to SEQ ID NO: 727. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 649 and encodes a GAA protein comprising the sequence set forth in SEQ ID NO: 727. In another example, the GAA coding sequence is (or comprises a sequence) at least 95%, at least 96%, at least 97%, at least 98%, at least 99%, at least 99.5%, or 100% identical to SEQ ID NO: 649. In another example, the GAA coding sequence is (or comprises a sequence) at least 95%, at least 96%, at least 97%, at least 98%, at least 99%, at least 99.5%, or 100% identical to SEQ ID NO: 649 and encodes a GAA protein (or a GAA protein comprising a sequence) at least 99%, at least 99.5%, or 100% identical to SEQ ID NO: 727. In another example, the GAA coding sequence is (or comprises a sequence) at least 95%, at least 96%, at least 97%, at least 98%, at least 99%, at least 99.5%, or 100% identical to SEQ ID NO: 649 and encodes a GAA protein comprising the sequence set forth in SEQ ID NO: 727. In another example, the GAA coding sequence is (or comprises a sequence) at least 99%, at least 99.5%, or 100% identical to SEQ ID NO: 649. In another example, the GAA coding sequence is (or comprises a sequence) at least 99%, at least 99.5%, or 100% identical to SEQ ID NO: 649 and encodes a GAA protein (or a GAA protein comprising a sequence) at least 99%, at least 99.5%, or 100% identical to SEQ ID NO: 727. In another example, the GAA coding sequence is (or comprises a sequence) at least 99%, at least 99.5%, or 100% identical to SEQ ID NO: 649 and encodes a GAA protein comprising the sequence set forth in SEQ ID NO: 727. In another example, the GAA coding sequence comprises the sequence set forth in SEQ ID NO: 649. In another example, the GAA coding sequence consists essentially of the sequence set forth in SEQ ID NO: 649. In another example, the GAA coding sequence consists of the sequence set forth in SEQ ID NO: 649. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 727 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 727 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 727 (and, e.g., retaining the activity of native GAA). Optionally, the GAA coding sequence in the above examples encodes a GAA protein comprising the sequence set forth in SEQ ID NO: 727. Optionally, the GAA coding sequence in the above examples encodes a GAA protein consisting essentially of the sequence set forth in SEQ ID NO: 727. Optionally, the GAA coding sequence in the above examples encodes a GAA protein consisting of the sequence set forth in SEQ ID NO: 727. In some embodiments, the nucleotide at position 1095 (or the corresponding position when the GAA coding sequence is aligned with SEQ ID NO: 750) is a “G.” In some embodiments, the nucleotide at position 1098 (or the corresponding position when the GAA coding sequence is aligned with SEQ ID NO: 750) is a “C.” In some embodiments, the nucleotide at position 2343 (or the corresponding position when the GAA coding sequence is aligned with SEQ ID NO: 750) is a “G.” In some embodiments, the nucleotide at position 1095 (or the corresponding position when the GAA coding sequence is aligned with SEQ ID NO: 750) is a “G,” the nucleotide at position 1098 (or the corresponding position when the GAA coding sequence is aligned with SEQ ID NO: 750) is a “C,” and the nucleotide at position 2343 (or the corresponding position when the GAA coding sequence is aligned with SEQ ID NO: 750) is a “G.”

Various other codon optimized GAA coding sequences are provided. The GAA coding sequence can be, for example, CpG-depleted (e.g., fully CpG depleted) and/or codon optimized (e.g., CpG depleted (e.g., fully CpG-depleted) and codon optimized).

When specific GAA or multidomain therapeutic protein nucleic acid constructs sequences are disclosed herein, they are meant to encompass the sequence disclosed or the reverse complement of the sequence. For example, if a GAA or multidomain therapeutic protein nucleic acid construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the GAA or multidomain therapeutic protein nucleic acid constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### (b) CD63-Binding Delivery Domain

The multidomain therapeutic proteins disclosed herein can comprise a CD63-binding delivery domain fused to a GAA polypeptide. The CD63-binding domain provides binding to the internalization factor CD63 (UniProt Ref. P08962-1). CD63 (also known as CD63 antigen, granulophysin, lysosomal-associated membrane protein 3, LAMP-3, lysosome integral membrane protein 1, Limp1, melanoma-associated antigen ME491, OMA81H, ocular melanoma-associated antigen, tetraspanin-30, or Tspan-30) is a member of the tetraspanin superfamily of cell surface proteins that span the cell membrane four times. It is encoded by the CD63 gene (also known as MLA1 or TSPAN30). CD63 is expressed in virtually all tissues and is thought to be involved in forming and stabilizing signaling complexes. CD63 localizes to the cell membrane, lysosomal membrane, and late endosomal membrane. CD63 is known to associate with integrins and may be involved in epithelial-mesenchymal transitioning.

In some multidomain therapeutic proteins, the CD63-binding delivery domain is an antibody, an antibody fragment or other antigen-binding protein. In some multidomain therapeutic proteins, the CD63-binding delivery domain is an antigen-binding protein. Examples of antigen-binding proteins include, for example, a receptor-fusion molecule, a trap molecule, a receptor-Fc fusion molecule, an antibody, an Fab fragment, an F(ab′)2 fragment, an Fd fragment, an Fv fragment, a single-chain Fv (scFv) molecule, a dAb fragment, an isolated complementarity determining region (CDR), a CDR3 peptide, a constrained FR3-CDR3-FR4 peptide, a domain-specific antibody, a single domain antibody, a domain-deleted antibody, a chimeric antibody, a CDR-grafted antibody, a diabody, a triabody, a tetrabody, a minibody, a nanobody, a monovalent nanobody, a bivalent nanobody, a small modular immunopharmaceutical (SMIP), a camelid antibody (VHH heavy chain homodimeric antibody), and a shark variable IgNAR domain. Examples of CD63-binding delivery domains can be found in WO 2013/138400, WO 2017/007796, WO 2017/190079, WO 2017/100467, WO 2018/226861, WO 2019/157224, and WO 2019/222663, each of which is herein incorporated by reference in its entirety for all purposes.

In a particular multidomain therapeutic protein, the CD63-binding delivery domain is an anti-CD63 scFv. In a specific example, the anti-CD63 scFv can comprise SEQ ID NO: 730 or can be at least 90%, at least 91%, at least 92%, at least 93%, at least 94%, at least 95%, at least 96%, at least 97%, at least 98%, at least 99%, or at least 99.5% identical to SEQ ID NO: 730. In another specific example, the anti-CD63 scFv can consist essentially of SEQ ID NO: 730. In another specific example, the anti-CD63 scFv can consist of SEQ ID NO: 730.

The CD63-binding delivery domain coding sequences in the constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a CD63-binding delivery domain coding sequence in a construct disclosed herein has one or more cryptic splice sites mutated or removed. In another example, a CD63-binding delivery domain coding sequence in a construct disclosed herein has all identified cryptic splice sites mutated or removed. In another example, a CD63-binding delivery domain coding sequence in a construct disclosed herein has one or more CpG dinucleotides removed (i.e., is CpG depleted). In another example, a CD63-binding delivery domain coding sequence in a construct disclosed herein has all CpG dinucleotides removed (i.e., is fully CpG depleted). In another example, a CD63-binding delivery domain coding sequence in a construct disclosed herein is codon optimized (e.g., codon optimized for expression in a human or mammal). In a specific example, a CD63-binding delivery domain coding sequence in a construct disclosed herein has one or more CpG dinucleotides removed (i.e., is CpG depleted) and has one or more cryptic splice sites mutated or removed. In another specific example, a CD63-binding delivery domain coding sequence in a construct disclosed herein has all CpG dinucleotides removed and has one or more or all identified cryptic splice sites mutated or removed. In another specific example, a CD63-binding delivery domain coding sequence in a construct disclosed herein has one or more CpG dinucleotides removed (i.e., is CpG depleted) and is codon optimized (e.g., codon optimized for expression in a human or mammal). In another specific example, a CD63-binding delivery domain coding sequence in a construct disclosed herein has all CpG dinucleotides removed (i.e., is fully CpG depleted) and is codon optimized (e.g., codon optimized for expression in a human or mammal).

Various anti-CD63 scFv coding sequences are provided. In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 759, 760, and 732. In another example, the anti-CD63 scFv coding sequence is (or comprises a sequence) at least 95%, at least 96%, at least 97%, at least 98%, at least 99%, at least 99.5%, or 100% identical to any one of SEQ ID NOS: 759, 760, and 732. In another example, the anti-CD63 scFv coding sequence is (or comprises a sequence) at least 99%, at least 99.5%, or 100% identical to any one of SEQ ID NOS: 759, 760, and 732. In another example, the anti-CD63 scFv coding sequence comprises the sequence set forth in any one of SEQ ID NOS: 759, 760, and 732. In another example, the anti-CD63 scFv coding sequence consists essentially of the sequence set forth in any one of SEQ ID NOS: 759, 760, and 732. In another example, the anti-CD63 scFv coding sequence consists of the sequence set forth in any one of SEQ ID NOS: 759, 760, and 732. In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 759. In another example, the anti-CD63 scFv coding sequence is (or comprises a sequence) at least 95%, at least 96%, at least 97%, at least 98%, at least 99%, at least 99.5%, or 100% identical to SEQ ID NO: 759. In another example, the anti-CD63 scFv coding sequence is (or comprises a sequence) at least 99%, at least 99.5%, or 100% identical to SEQ ID NO: 759. In another example, the anti-CD63 scFv coding sequence comprises the sequence set forth in SEQ ID NO: 759. In another example, the anti-CD63 scFv coding sequence consists essentially of the sequence set forth in SEQ ID NO: 759. In another example, the anti-CD63 scFv coding sequence consists of the sequence set forth in SEQ ID NO: 759. Optionally, the anti-CD63 scFv coding sequence encodes an anti-CD63 scFv protein (or an anti-CD63 scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 730 (and, e.g., retaining CD63-binding activity). Optionally, the anti-CD63 scFv coding sequence encodes an anti-CD63 scFv protein (or an anti-CD63 scFv protein comprising a sequence) at least 95%, at least 96%, at least 97%, at least 98%, at least 99%, at least 99.5%, or 100% identical to SEQ ID NO: 730 (and, e.g., retaining CD63-binding activity). Optionally, the anti-CD63 scFv coding sequence in the above examples encodes an anti-CD63 scFv protein (or an anti-CD63 scFv protein comprising a sequence) at least 99%, at least 99.5%, or 100% identical to SEQ ID NO: 730 (and, e.g., retaining CD63-binding activity). Optionally, the anti-CD63 scFv coding sequence in the above examples encodes an anti-CD63 scFv protein comprising the sequence set forth in SEQ ID NO: 730. Optionally, the anti-CD63 scFv coding sequence in the above examples encodes an anti-CD63 scFv protein consisting essentially of the sequence set forth in SEQ ID NO: 730. Optionally, the anti-CD63 scFv coding sequence in the above examples encodes an anti-CD63 scFv protein consisting of the sequence set forth in SEQ ID NO: 730. In some embodiments, the nucleotide at position 3 (or the corresponding position when the anti-CD63 scFv coding sequence is aligned with SEQ ID NO: 759) is an “A.” In some embodiments, the nucleotide at position 132 (or the corresponding position when the anti-CD63 scFv coding sequence is aligned with SEQ ID NO: 759) is an “A.” In some embodiments, the nucleotide at position 273 (or the corresponding position when the anti-CD63 scFv coding sequence is aligned with SEQ ID NO: 759) is a “T.” In some embodiments, the nucleotide at position 3 (or the corresponding position when the anti-CD63 scFv coding sequence is aligned with SEQ ID NO: 759) is an “A,” the nucleotide at position 132 (or the corresponding position when the anti-CD63 scFv coding sequence is aligned with SEQ ID NO: 759) is an “A,” and the nucleotide at position 273 (or the corresponding position when the anti-CD63 scFv coding sequence is aligned with SEQ ID NO: 759) is a “T.”

In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 759. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 759 and encodes an anti-CD63 scFv protein (or an anti-CD63 scFv protein comprising a sequence) at least 99%, at least 99.5%, or 100% identical to SEQ ID NO: 730. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 759 and encodes an anti-CD63 scFv protein comprising the sequence set forth in SEQ ID NO: 730. In another example, the anti-CD63 scFv coding sequence is (or comprises a sequence) at least 95%, at least 96%, at least 97%, at least 98%, at least 99%, at least 99.5%, or 100% identical to SEQ ID NO: 759. In another example, the anti-CD63 scFv coding sequence is (or comprises a sequence) at least 95%, at least 96%, at least 97%, at least 98%, at least 99%, at least 99.5%, or 100% identical to SEQ ID NO: 759 and encodes an anti-CD63 scFv protein (or an anti-CD63 scFv protein comprising a sequence) at least 99%, at least 99.5%, or 100% identical to SEQ ID NO: 730. In another example, the anti-CD63 scFv coding sequence is (or comprises a sequence) at least 95%, at least 96%, at least 97%, at least 98%, at least 99%, at least 99.5%, or 100% identical to SEQ ID NO: 759 and encodes an anti-CD63 scFv protein comprising the sequence set forth in SEQ ID NO: 730. In another example, the anti-CD63 scFv coding sequence is (or comprises a sequence) at least 99%, at least 99.5%, or 100% identical to SEQ ID NO: 759. In another example, the anti-CD63 scFv coding sequence is (or comprises a sequence) at least 99%, at least 99.5%, or 100% identical to SEQ ID NO: 759 and encodes an anti-CD63 scFv protein (or an anti-CD63 scFv protein comprising a sequence) at least 99%, at least 99.5%, or 100% identical to SEQ ID NO: 730. In another example, the anti-CD63 scFv coding sequence is (or comprises a sequence) at least 99%, at least 99.5%, or 100% identical to SEQ ID NO: 759 and encodes an anti-CD63 scFv protein comprising the sequence set forth in SEQ ID NO: 730. In another example, the anti-CD63 scFv coding sequence comprises the sequence set forth in SEQ ID NO: 759. In another example, the anti-CD63 scFv coding sequence consists essentially of the sequence set forth in SEQ ID NO: 759. In another example, the anti-CD63 scFv coding sequence consists of the sequence set forth in SEQ ID NO: 759. The anti-CD63 scFv coding sequence can be, for example, CpG-depleted (e.g., fully CpG-depleted) and/or codon optimized. For example, the anti-CD63 scFv coding sequence can be CpG depleted (e.g., fully CpG-depleted) and codon optimized. Optionally, the anti-CD63 scFv coding sequence encodes an anti-CD63 scFv protein (or an anti-CD63 scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 730 (and, e.g., retaining CD63-binding activity). Optionally, the anti-CD63 scFv coding sequence encodes an anti-CD63 scFv protein (or an anti-CD63 scFv protein comprising a sequence) at least 95%, at least 96%, at least 97%, at least 98%, at least 99%, at least 99.5%, or 100% identical to SEQ ID NO: 730 (and, e.g., retaining CD63-binding activity). Optionally, the anti-CD63 scFv coding sequence in the above examples encodes an anti-CD63 scFv protein (or an anti-CD63 scFv protein comprising a sequence) at least 99%, at least 99.5%, or 100% identical to SEQ ID NO: 730 (and, e.g., retaining CD63-binding activity). Optionally, the anti-CD63 scFv coding sequence in the above examples encodes an anti-CD63 scFv protein comprising the sequence set forth in SEQ ID NO: 730. Optionally, the anti-CD63 scFv coding sequence in the above examples encodes an anti-CD63 scFv protein consisting essentially of the sequence set forth in SEQ ID NO: 730. Optionally, the anti-CD63 scFv coding sequence in the above examples encodes an anti-CD63 scFv protein consisting of the sequence set forth in SEQ ID NO: 730. In some embodiments, the nucleotide at position 3 (or the corresponding position when the anti-CD63 scFv coding sequence is aligned with SEQ ID NO: 759) is an “A.” In some embodiments, the nucleotide at position 132 (or the corresponding position when the anti-CD63 scFv coding sequence is aligned with SEQ ID NO: 759) is an “A.” In some embodiments, the nucleotide at position 273 (or the corresponding position when the anti-CD63 scFv coding sequence is aligned with SEQ ID NO: 759) is a “T.” In some embodiments, the nucleotide at position 3 (or the corresponding position when the anti-CD63 scFv coding sequence is aligned with SEQ ID NO: 759) is an “A,” the nucleotide at position 132 (or the corresponding position when the anti-CD63 scFv coding sequence is aligned with SEQ ID NO: 759) is an “A,” and the nucleotide at position 273 (or the corresponding position when the anti-CD63 scFv coding sequence is aligned with SEQ ID NO: 759) is a “T.”

In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 760. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 760 and encodes an anti-CD63 scFv protein (or an anti-CD63 scFv protein comprising a sequence) at least 99%, at least 99.5%, or 100% identical to SEQ ID NO: 730. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 760 and encodes an anti-CD63 scFv protein comprising the sequence set forth in SEQ ID NO: 730. In another example, the anti-CD63 scFv coding sequence is (or comprises a sequence) at least 95%, at least 96%, at least 97%, at least 98%, at least 99%, at least 99.5%, or 100% identical to SEQ ID NO: 760. In another example, the anti-CD63 scFv coding sequence is (or comprises a sequence) at least 95%, at least 96%, at least 97%, at least 98%, at least 99%, at least 99.5%, or 100% identical to SEQ ID NO: 760 and encodes an anti-CD63 scFv protein (or an anti-CD63 scFv protein comprising a sequence) at least 99%, at least 99.5%, or 100% identical to SEQ ID NO: 730. In another example, the anti-CD63 scFv coding sequence is (or comprises a sequence) at least 95%, at least 96%, at least 97%, at least 98%, at least 99%, at least 99.5%, or 100% identical to SEQ ID NO: 760 and encodes an anti-CD63 scFv protein comprising the sequence set forth in SEQ ID NO: 730. In another example, the anti-CD63 scFv coding sequence is (or comprises a sequence) at least 99%, at least 99.5%, or 100% identical to SEQ ID NO: 760. In another example, the anti-CD63 scFv coding sequence is (or comprises a sequence) at least 99%, at least 99.5%, or 100% identical to SEQ ID NO: 760 and encodes an anti-CD63 scFv protein (or an anti-CD63 scFv protein comprising a sequence) at least 99%, at least 99.5%, or 100% identical to SEQ ID NO: 730. In another example, the anti-CD63 scFv coding sequence is (or comprises a sequence) at least 99%, at least 99.5%, or 100% identical to SEQ ID NO: 760 and encodes an anti-CD63 scFv protein comprising the sequence set forth in SEQ ID NO: 730. In another example, the anti-CD63 scFv coding sequence comprises the sequence set forth in SEQ ID NO: 760. In another example, the anti-CD63 scFv coding sequence consists essentially of the sequence set forth in SEQ ID NO: 760. In another example, the anti-CD63 scFv coding sequence consists of the sequence set forth in SEQ ID NO: 760. The anti-CD63 scFv coding sequence can be, for example, CpG-depleted (e.g., fully CpG-depleted) and/or codon optimized. For example, the anti-CD63 scFv coding sequence can be CpG depleted (e.g., fully CpG-depleted) and codon optimized. Optionally, the anti-CD63 scFv coding sequence encodes an anti-CD63 scFv protein (or an anti-CD63 scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 730 (and, e.g., retaining CD63-binding activity). Optionally, the anti-CD63 scFv coding sequence encodes an anti-CD63 scFv protein (or an anti-CD63 scFv protein comprising a sequence) at least 95%, at least 96%, at least 97%, at least 98%, at least 99%, at least 99.5%, or 100% identical to SEQ ID NO: 730 (and, e.g., retaining CD63-binding activity). Optionally, the anti-CD63 scFv coding sequence in the above examples encodes an anti-CD63 scFv protein (or an anti-CD63 scFv protein comprising a sequence) at least 99%, at least 99.5%, or 100% identical to SEQ ID NO: 730 (and, e.g., retaining CD63-binding activity). Optionally, the anti-CD63 scFv coding sequence in the above examples encodes an anti-CD63 scFv protein comprising the sequence set forth in SEQ ID NO: 730. Optionally, the anti-CD63 scFv coding sequence in the above examples encodes an anti-CD63 scFv protein consisting essentially of the sequence set forth in SEQ ID NO: 730. Optionally, the anti-CD63 scFv coding sequence in the above examples encodes an anti-CD63 scFv protein consisting of the sequence set forth in SEQ ID NO: 730. In some embodiments, the nucleotide at position 3 (or the corresponding position when the anti-CD63 scFv coding sequence is aligned with SEQ ID NO: 759) is an “A.” In some embodiments, the nucleotide at position 132 (or the corresponding position when the anti-CD63 scFv coding sequence is aligned with SEQ ID NO: 759) is an “A.” In some embodiments, the nucleotide at position 273 (or the corresponding position when the anti-CD63 scFv coding sequence is aligned with SEQ ID NO: 759) is a “T.” In some embodiments, the nucleotide at position 3 (or the corresponding position when the anti-CD63 scFv coding sequence is aligned with SEQ ID NO: 759) is an “A,” the nucleotide at position 132 (or the corresponding position when the anti-CD63 scFv coding sequence is aligned with SEQ ID NO: 759) is an “A,” and the nucleotide at position 273 (or the corresponding position when the anti-CD63 scFv coding sequence is aligned with SEQ ID NO: 759) is a “T.”

In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 732. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 732 and encodes an anti-CD63 scFv protein (or an anti-CD63 scFv protein comprising a sequence) at least 99%, at least 99.5%, or 100% identical to SEQ ID NO: 730. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 732 and encodes an anti-CD63 scFv protein comprising the sequence set forth in SEQ ID NO: 730. In another example, the anti-CD63 scFv coding sequence is (or comprises a sequence) at least 95%, at least 96%, at least 97%, at least 98%, at least 99%, at least 99.5%, or 100% identical to SEQ ID NO: 732. In another example, the anti-CD63 scFv coding sequence is (or comprises a sequence) at least 95%, at least 96%, at least 97%, at least 98%, at least 99%, at least 99.5%, or 100% identical to SEQ ID NO: 732 and encodes an anti-CD63 scFv protein (or an anti-CD63 scFv protein comprising a sequence) at least 99%, at least 99.5%, or 100% identical to SEQ ID NO: 730. In another example, the anti-CD63 scFv coding sequence is (or comprises a sequence) at least 95%, at least 96%, at least 97%, at least 98%, at least 99%, at least 99.5%, or 100% identical to SEQ ID NO: 732 and encodes an anti-CD63 scFv protein comprising the sequence set forth in SEQ ID NO: 730. In another example, the anti-CD63 scFv coding sequence is (or comprises a sequence) at least 99%, at least 99.5%, or 100% identical to SEQ ID NO: 732. In another example, the anti-CD63 scFv coding sequence is (or comprises a sequence) at least 99%, at least 99.5%, or 100% identical to SEQ ID NO: 732 and encodes an anti-CD63 scFv protein (or an anti-CD63 scFv protein comprising a sequence) at least 99%, at least 99.5%, or 100% identical to SEQ ID NO: 730. In another example, the anti-CD63 scFv coding sequence is (or comprises a sequence) at least 99%, at least 99.5%, or 100% identical to SEQ ID NO: 732 and encodes an anti-CD63 scFv protein comprising the sequence set forth in SEQ ID NO: 730. In another example, the anti-CD63 scFv coding sequence comprises the sequence set forth in SEQ ID NO: 732. In another example, the anti-CD63 scFv coding sequence consists essentially of the sequence set forth in SEQ ID NO: 732. In another example, the anti-CD63 scFv coding sequence consists of the sequence set forth in SEQ ID NO: 732. The anti-CD63 scFv coding sequence can be, for example, CpG-depleted (e.g., fully CpG-depleted) and/or codon optimized. For example, the anti-CD63 scFv coding sequence can be CpG depleted (e.g., fully CpG-depleted) and codon optimized. Optionally, the anti-CD63 scFv coding sequence encodes an anti-CD63 scFv protein (or an anti-CD63 scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 730 (and, e.g., retaining CD63-binding activity). Optionally, the anti-CD63 scFv coding sequence encodes an anti-CD63 scFv protein (or an anti-CD63 scFv protein comprising a sequence) at least 95%, at least 96%, at least 97%, at least 98%, at least 99%, at least 99.5%, or 100% identical to SEQ ID NO: 730 (and, e.g., retaining CD63-binding activity). Optionally, the anti-CD63 scFv coding sequence in the above examples encodes an anti-CD63 scFv protein (or an anti-CD63 scFv protein comprising a sequence) at least 99%, at least 99.5%, or 100% identical to SEQ ID NO: 730 (and, e.g., retaining CD63-binding activity). Optionally, the anti-CD63 scFv coding sequence in the above examples encodes an anti-CD63 scFv protein comprising the sequence set forth in SEQ ID NO: 730. Optionally, the anti-CD63 scFv coding sequence in the above examples encodes an anti-CD63 scFv protein consisting essentially of the sequence set forth in SEQ ID NO: 730. Optionally, the anti-CD63 scFv coding sequence in the above examples encodes an anti-CD63 scFv protein consisting of the sequence set forth in SEQ ID NO: 730. In some embodiments, the nucleotide at position 3 (or the corresponding position when the anti-CD63 scFv coding sequence is aligned with SEQ ID NO: 759) is an “A.” In some embodiments, the nucleotide at position 132 (or the corresponding position when the anti-CD63 scFv coding sequence is aligned with SEQ ID NO: 759) is an “A.” In some embodiments, the nucleotide at position 273 (or the corresponding position when the anti-CD63 scFv coding sequence is aligned with SEQ ID NO: 759) is a “T.” In some embodiments, the nucleotide at position 3 (or the corresponding position when the anti-CD63 scFv coding sequence is aligned with SEQ ID NO: 759) is an “A,” the nucleotide at position 132 (or the corresponding position when the anti-CD63 scFv coding sequence is aligned with SEQ ID NO: 759) is an “A,” and the nucleotide at position 273 (or the corresponding position when the anti-CD63 scFv coding sequence is aligned with SEQ ID NO: 759) is a “T.”

When specific anti-CD63 scFv or multidomain therapeutic protein nucleic acid constructs sequences are disclosed herein, they are meant to encompass the sequence disclosed or the reverse complement of the sequence. For example, if an anti-CD63 scFv or multidomain therapeutic protein nucleic acid construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the anti-CD63 scFv or multidomain therapeutic protein nucleic acid constructs are part of a single-stranded recombinant AAV vector. Single-stranded AAV genomes are packaged as either sense (plus-stranded) or anti-sense (minus-stranded genomes), and single-stranded AAV genomes of + and -polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### (c) TfR-Binding Delivery Domain

The multidomain therapeutic proteins disclosed herein can comprise a TfR-binding delivery domain fused to a GAA polypeptide. The TfR-binding domain provides binding to the internalization factor transferrin receptor protein 1(TfR; UniProt Ref. P02786). TfR (also known as TR, TfR1, and Trfr) is encoded by the TFRC gene. TfR is expressed in muscle and on brain endothelial cells. Transcytosis of TfR in these cells enables blood-brain-barrier crossing. In some embodiments, the multidomain therapeutic proteins comprising a TfR-binding delivery domain (e.g., scFv) fused to a GAA polypeptide do not alter transferrin uptake. In some embodiments, the multidomain therapeutic proteins comprising a TfR-binding delivery domain (e.g., scFv) fused to a GAA polypeptide do not alter iron homeostasis. In some embodiments, the multidomain therapeutic proteins comprising a TfR-binding delivery domain (e.g., scFv) fused to a GAA polypeptide do not alter transferrin uptake or iron homeostasis.

Transferrin receptor 1 (TfR) is a membrane receptor involved in the control of iron supply to the cell through the binding of transferrin, the major iron-carrier protein. Transferrin receptor 1 is expressed from the TFRC gene. Transferrin receptor 1 may be referred to, herein, at TFRC. This receptor plays a key role in the control of cell proliferation because iron is essential for sustaining ribonucleotide reductase activity, and is the only enzyme that catalyzes the conversion of ribonucleotides to deoxyribonucleotides. Preferably, the TfR is human TfR (hTfR). See e.g., Accession numbers NP_001121620.1; BAD92491.1; and NP_001300894.1; and e!Ensembl entry: ENSG00000072274. The human transferrin receptor 1 is expressed in several tissues, including but not limited to: cerebral cortex; cerebellum; hippocampus; caudate; parathyroid gland; adrenal gland; bronchus; lung; oral mucosa; esophagus; stomach; duodenum; small intestine; colon; rectum; liver; gallbladder; pancreas; kidney; urinary bladder; testis; epididymis; prostate; vagina; ovary; fallopian tube; endometrium; cervix; placenta; breast; heart muscle; smooth muscle; soft tissue; skin; appendix; lymph node; tonsil; and bone marrow. A related transferrin receptor is transferrin receptor 2 (TfR2). Human transferrin receptor 2 bears about 45% sequence identity to human transferrin receptor 1. Trinder & Baker, Transferrin receptor 2: a new molecule in iron metabolism. Int J Biochem Cell Biol. 2003 March; 35(3):292-6. Unless otherwise stated, transferrin receptor as used herein generally refers to transferrin receptor 1 (e.g., human transferrin receptor 1).

Human Transferrin (Tf) is a single chain, 80 kDa member of the anion-binding superfamily of proteins. Transferrin is a 698 amino acid precursor that is divided into a 19 aa signal sequence plus a 679 aa mature segment that typically contains 19 intrachain disulfide bonds. The N- and C-terminal flanking regions (or domains) bind ferric iron through the interaction of an obligate anion (e.g., bicarbonate) and four amino acids (His, Asp, and two Tyr). Apotransferrin (or iron-free) will initially bind one atom of iron at the C-terminus, and this is followed by subsequent iron binding by the N-terminus to form holotransferrin (diferric Tf, Holo-Tf). Through its C-terminal iron-binding domain, holotransferrin will interact with the TfR on the surface of cells where it is internalized into acidified endosomes. Iron dissociates from the Tf molecule within these endosomes, and is transported into the cytosol as ferrous iron. In addition to TfR, transferrin is reported to bind to cubulin, IGFBP3, microbial iron-binding proteins and liver-specific TfR2.

The blood-brain barrier (BBB) is located within the microvasculature of the brain, and it regulates passage of molecules from the blood to the brain. Burkhart et al., Accessing targeted nanoparticles to the brain: the vascular route. Curr Med Chem. 2014; 21(36):4092-9. The transcellular passage through the brain capillary endothelial cells can take place via 1) cell entry by leukocytes; 2) carrier-mediated influx of e.g., glucose by glucose transporter 1 (GLUT-1), amino acids by e.g., the L-type amino acid transporter 1 (LAT-1) and small peptides by e.g., organic anion-transporting peptide-B (OATP-B); 3) paracellular passage of small hydrophobic molecules; 4) adsorption-mediated transcytosis of e.g., albumin and cationized molecules; 5) passive diffusion of lipid soluble, non-polar solutes, including CO₂ and O₂; and 5) receptor-mediated transcytosis of, e.g., insulin by the insulin receptor and Tf by the TfR. Johnsen et al., Targeting the transferrin receptor for brain drug delivery, Prog Neurobiol. 2019 October; 181:101665.

For example, anti-TfR:GAA fusion proteins exhibiting high affinity to the transferrin receptor and superior blood-brain barrier crossing are provided. Surprisingly, fusions exhibiting high binding affinity to TfR crossed the blood-brain barrier more efficiently than that of low affinity binders. We found that high affinity antibodies impart the best delivery to the CNS and muscle in the anti-hTFRscfv:GAA format. This is in contrast to previous findings with mono- and bivalent anti-TFR antibodies, where low affinity antibodies crossed the BBB more effectively. The fusions provided herein have an ability to efficiently deliver GAA to the brain and, thus, are an effective treatment of diseases such as GAA deficiency (e.g., Pompe disease).

Provided herein are antigen-binding proteins, such as antibodies, antigen-binding fragments thereof, such as Fabs and scFvs, that bind specifically to the transferrin receptor, preferably the human transferrin receptor 1 (anti-hTfR). For example, in an embodiment, the anti-hTfR is in the form of a fusion protein. The fusion protein includes the anti-hTfR antigen-binding protein fused to a GAA polypeptide. The anti-hTfRs efficiently cross the blood-brain barrier (BBB) and can, thereby, deliver the fused GAA to the brain.

An antigen-binding protein that specifically binds to transferrin receptor and fusions thereof, for example, a tag such as His6 and/or myc (e.g., human transferrin receptor (e.g., REGN2431) or monkey transferrin receptor (e.g., REGN2054)) binds at about 25° C., e.g., in a surface plasmon resonance assay, with a K_{D} of about 20 nM or a higher affinity. Such an antigen-binding protein may be referred to as “anti-TfR.” In some embodiments, the antigen-binding protein binds to human transferrin receptor with a K_{D} of about 0.41 nM or a stronger affinity. In some embodiments, the antigen-binding protein binds to human transferrin receptor with a K_{D} of about 3 nM or a stronger affinity. In some embodiments, the antigen-binding protein binds to human transferrin receptor with a K_{D} of about 0.45 nM to 3 nM. In some embodiments, a Fab having an HCVR and LCVR binds to human transferrin receptor with a K_{D} of about 0.65 nM or a stronger affinity. In some embodiments, a fusion protein disclosed herein binds to human transferrin receptor with a K_{D} of about 1×10⁻⁷ M or a stronger affinity.

In an embodiment, an anti-hTfR scFv:GAA fusion protein includes an scFv comprising the arrangement of variable regions as follows: LCVR-HCVR or HCVR-LCVR, wherein the HCVR and LCVR are optionally connected by a linker and the scFv is connected, optionally by a linker, to a GAA polypeptide (e.g., LCVR-(Gly₄Ser)₃(SEQ ID NO: 616)-HCVR-(Gly₄Ser)₂(SEQ ID NO: 617))-GAA; or LCVR-(Gly₄Ser)₃(SEQ ID NO: 616)-HCVR-(Gly₄Ser)₂(SEQ ID NO: 617))-GAA) (Gly₄Ser=SEQ ID NO: 537)). In one example, an scFv comprises an arrangement of variable regions as follows: LCVR-HCVR. In another example, an scFv comprises an arrangement of variable regions as follows: HCVR-LCVR. In one example, the linker between the HCVR and LCVR comprises, consists essentially of, or consists of three such repeats (SEQ ID NO: 616). For example, the coding sequence for the linker can comprise, consist essentially of, or consist of any one of SEQ ID NOS: 618-622 and 747. In another example, the linker between the HCVR and LCVR comprises, consists essentially of, or consists of two such repeats (SEQ ID NO: 617). For example, the coding sequence for the linker can comprise, consist essentially of, or consist of any one of SEQ ID NOS: 623-629. In another example, the linker between the HCVR and LCVR comprises, consists essentially of, or consists of one such repeat (SEQ ID NO: 537). For example, the coding sequence for the linker can comprise, consist essentially of, or consist of SEQ ID NO: 630 or 748. In one example, the linker between the scFv and GAA comprises, consists essentially of, or consists of three such repeats (SEQ ID NO: 616). For example, the coding sequence for the linker can comprise, consist essentially of, or consist of any one of SEQ ID NOS: 618-622 and 747. In another example, the linker between the scFv and GAA comprises, consists essentially of, or consists of two such repeats (SEQ ID NO: 617). For example, the coding sequence for the linker can comprise, consist essentially of, or consist of any one of SEQ ID NOS: 623-629. In another example, the linker between the scFv and GAA comprises, consists essentially of, or consists of one such repeat (SEQ ID NO: 537). For example, the coding sequence for the linker can comprise, consist essentially of, or consist of SEQ ID NO: 630 or 748. In another example, a rigid linker can be used such as a 2×H4 linker. In one example, the linker comprises, consists essentially of, or consists of AEAAAKEAAAKEAAAKEAAAKALEAEAAAKEAAAKEAAAKEAAAKA (SEQ ID NO: 842). For example, the coding sequence for the linker can comprise, consist essentially of, or consist of SEQ ID NO: 841.

An anti-hTfR:GAA optionally comprises a signal peptide, connected to the antigen-binding protein that binds specifically to transferrin receptor (TfR), preferably, human transferrin receptor (hTfR) which is fused (optionally by a linker) to GAA. In an embodiment, the signal peptide is the mROR signal sequence (e.g., mROR signal sequence-LCVR-(Gly₄Ser)₃(SEQ ID NO: 616)-HCVR-(Gly₄Ser)₂(SEQ ID NO: 617))-GAA; or LCVR-(Gly₄Ser)₃(SEQ ID NO: 616)-HCVR-(Gly₄Ser)₂(SEQ ID NO: 617))-GAA) (Gly₄Ser=SEQ ID NO: 537)). The term “fused” or “tethered” with regard to fused polypeptides refers to polypeptides joined directly or indirectly (e.g., via a linker or other polypeptide).

In an embodiment, the assignment of amino acids to each framework or CDR domain in an immunoglobulin is in accordance with the definitions of Sequences of Proteins of Immunological Interest, Kabat et al.; National Institutes of Health, Bethesda, Md.; 5^{th} ed.; NIH Publ. No. 91-3242 (1991); Kabat (1978) Adv. Prot. Chem. 32:1-75; Kabat et al., (1977) J. Biol. Chem. 252:6609-6616; Chothia, et al., (1987) J Mol. Biol. 196:901-917 or Chothia, et al., (1989) Nature 342: 878-883. Thus, included are antibodies and antigen-binding fragments including the CDRs of a V_{H} and the CDRs of a V_{L}, which V_{H} and V_{L} comprise amino acid sequences as set forth herein (see, e.g., sequences of Table 2, or variants thereof), wherein the CDRs are as defined according to Kabat and/or Chothia.

In some multidomain therapeutic proteins, the TfR-binding delivery domain is an antibody, an antibody fragment or other antigen-binding protein. In some multidomain therapeutic proteins, the TfR-binding delivery domain is an antigen-binding protein. Examples of antigen-binding proteins include, for example, a receptor-fusion molecule, a trap molecule, a receptor-Fc fusion molecule, an antibody, an Fab fragment, an F(ab′)₂ fragment, an Fd fragment, an Fv fragment, a single-chain Fv (scFv) molecule, a dAb fragment, an isolated complementarity determining region (CDR), a CDR3 peptide, a constrained FR3-CDR3-FR4 peptide, a domain-specific antibody, a single domain antibody, a domain-deleted antibody, a chimeric antibody, a CDR-grafted antibody, a diabody, a triabody, a tetrabody, a minibody, a nanobody, a monovalent nanobody, a bivalent nanobody, a small modular immunopharmaceutical (SMIP), a camelid antibody (VHH heavy chain homodimeric antibody), and a shark variable IgNAR domain.

Provided herein are antibodies that bind specifically to the human transferrin receptor 1. The term “antibody,” as used herein, refers to immunoglobulin molecules comprising four polypeptide chains, two heavy chains (HCs) and two light chains (LCs), inter-connected by disulfide bonds. In an embodiment, each antibody heavy chain (HC) comprises a heavy chain variable region (“HCVR” or “V_{H}”) (e.g., comprising SEQ ID NO: 171, 680, 181, 681, 191, 682, 201, 211, 221, 685, 231, 687, 241, 689, 251, 261, 691, 271, 281, 692, 291, 301, 311, 694, 321, 331, 696, 341, 351, 697, 361, 699, 371, 700, 381, 391, 401, 411, 421, 701, 431, 441, 451, 461, 471, 702, and/or 481 or a variant thereof) and a heavy chain constant region (e.g., human IgG, human IgG1 or human IgG4); and each antibody light chain (LC) comprises a light chain variable region (“LCVR or “V_{L}”) (e.g., SEQ ID NO: 176, 186, 196, 206, 683, 216, 684, 226, 686, 236, 688, 246, 690, 256, 266, 276, 286, 693, 296, 306, 316, 695, 326, 336, 346, 356, 698, 366, 376, 386, 396, 406, 416, 426, 436, 446, 456, 466, 476, 632, 486, and/or 703 or a variant thereof) and a light chain constant region (e.g., human kappa or human lambda). In an embodiment, each antibody heavy chain (HC) comprises a heavy chain variable region (“HCVR” or “V_{H}”) (e.g., comprising SEQ ID NO: 391 or 411, or a variant thereof) and a heavy chain constant region (e.g., human IgG, human IgG1 or human IgG4); and each antibody light chain (LC) comprises a light chain variable region (“LCVR or “V_{L}”) (e.g., SEQ ID NO: 396 or 416, or a variant thereof) and a light chain constant region (e.g., human kappa or human lambda). In an embodiment, each antibody heavy chain (HC) comprises a heavy chain variable region (“HCVR” or “V_{H}”) (e.g., comprising SEQ ID NO: 391, or a variant thereof) and a heavy chain constant region (e.g., human IgG, human IgG1 or human IgG4); and each antibody light chain (LC) comprises a light chain variable region (“LCVR or “V_{L}”) (e.g., SEQ ID NO: 396, or a variant thereof) and a light chain constant region (e.g., human kappa or human lambda). In an embodiment, each antibody heavy chain (HC) comprises a heavy chain variable region (“HCVR” or “V_{H}”) (e.g., comprising SEQ ID NO: 411, or a variant thereof) and a heavy chain constant region (e.g., human IgG, human IgG1 or human IgG4); and each antibody light chain (LC) comprises a light chain variable region (“LCVR or “V_{L}”) (e.g., SEQ ID NO: 416, or a variant thereof) and a light chain constant region (e.g., human kappa or human lambda). The V_{H} and V_{L} regions can be further subdivided into regions of hypervariability, termed complementarity determining regions (CDR), interspersed with regions that are more conserved, termed framework regions (FR). Each V_{H} and V_{L} comprises three CDRs and four FRs. Anti-TfR antibodies disclosed herein can also be fused to GAA.

An anti-TfR antigen-binding protein provided herein may be an antigen-binding fragment of an antibody which may be tethered to GAA. The terms “antigen-binding portion” or “antigen-binding fragment” of an antibody, as used herein, refers to an immunoglobulin molecule that binds antigen but that does not include all of the sequences of a full antibody (preferably, the full antibody is an IgG). Non-limiting examples of antigen-binding fragments include: (i) Fab fragments; (ii) F(ab′)₂ fragments; (iii) Fd fragments; (iv) Fv fragments; (v) single-chain Fv (scFv) molecules; and (vi) dAb fragments; consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies and small modular immunopharmaceuticals (SMIPs), are also encompassed within the expression “antigen-binding fragment,” as used herein.

An anti-TfR antigen-binding protein may be an scFv which may be tethered to a GAA polypeptide. An scFv (single chain fragment variable) has variable regions of heavy (V_{H}) and light (V_{L}) domains (in either order), which, preferably, are joined together by a flexible linker (e.g., peptide linker). The length of the flexible linker used to link both of the V regions may be important for yielding the correct folding of the polypeptide chain. Previously, it has been estimated that the peptide linker must span 3.5 nm (35 Å) between the carboxy terminus of the variable domain and the amino terminus of the other domain without affecting the ability of the domains to fold and form an intact antigen-binding site (Huston et al., Protein engineering of single-chain Fv analogs and fusion proteins. Methods in Enzymology. 1991; 203:46-88). In an embodiment, the linker comprises an amino acid sequence of such length to separate the variable domains by about 3.5 nm.

In some embodiments, an anti-TfR antigen-binding protein described herein comprises a monovalent or “one-armed” antibody. The monovalent or “one-armed” antibodies as used herein refer to immunoglobulin proteins comprising a single variable domain. For example, the one-armed antibody may comprise a single variable domain within a Fab wherein the Fab is linked to at least one Fc fragment. In certain embodiments, the one-armed antibody comprises: (i) a heavy chain comprising a heavy chain constant region and a heavy chain variable region, (ii) a light chain comprising a light chain constant region and a light chain variable region, and (iii) a polypeptide comprising a Fc fragment or a truncated heavy chain. In certain embodiments, the Fc fragment or a truncated heavy chain comprised in the separate polypeptide is a “dummy Fe,” which refers to an Fc fragment that is not linked to an antigen binding domain. The one-armed antibodies of the present disclosure may comprise any of the HCVR/LCVR pairs or CDR amino acid sequences as set forth in Table 2 herein. One-armed antibodies comprising a full-length heavy chain, a full-length light chain and an additional Fc domain polypeptide can be constructed using standard methodologies (see, e.g., WO2010151792, which is incorporated herein by reference in its entirety), wherein the heavy chain constant region differs from the Fc domain polypeptide by at least two amino acids (e.g., H95R and Y96F according to the IMGT exon numbering system; or H435R and Y436F according to the EU numbering system). Such modifications are useful in purification of the monovalent antibodies (see WO2010151792).

An antigen-binding fragment of an antibody will, in an embodiment, comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody described herein include: (i) V_{H}-CH1; (ii) V_{H}-CH2; (iii) V_{H}-CH3; (iv) V_{H}-CH1-CH2; (v) V_{H}-CH1-CH2-CH3; (vi) V_{H}-CH2-CH3; (vii) V_{H}-CL; (viii) V_{L}-CH1; (ix) V_{L}-CH2; (x) V_{L}-CH3; (xi) V_{L}-CH1-CH2; (xii) V_{L}-CH1-CH2-CH3; (xiii) V_{L}-CH2-CH3; and (xiv) V_{L}-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody described herein may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)). The present disclosure includes an antigen-binding fragment of an antigen-binding protein such as an antibody set forth herein.

Antigen-binding proteins (e.g., antibodies and antigen-binding fragments) may be monospecific or multi-specific (e.g., bispecific). Multispecific antigen-binding proteins are discussed further herein. The present disclosure includes monospecific as well as multispecific (e.g., bispecific) antigen-binding fragments comprising one or more variable domains from an antigen-binding protein that is specifically set forth herein.

The term “specifically binds” or “binds specifically” refers to those antigen-binding proteins (e.g., antibodies or antigen-binding fragments thereof) having a binding affinity to an antigen, such as human TfR protein, mouse TfR protein or monkey TfR protein, expressed as K_{D}, of at least about 10⁻⁹ M (e.g., 0.01, 0.1, 0.2, 0.3, 0.4, 0.5, 0.6, 0.7, 0.8, 0.9 or 1.0 nM), as measured by real-time, label free bio-layer interferometry assay, for example, at 25° C. or 37° C., e.g., an Octet® HTX biosensor, or by surface plasmon resonance, e.g., BIACORE™, or by solution-affinity ELISA. The present disclosure includes antigen-binding proteins that specifically bind to TfR protein. “Anti-TfR” refers to an antigen-binding protein (or other molecule), for example, an antibody or antigen-binding fragment thereof, that binds specifically to TfR.

“Isolated” antigen-binding proteins (e.g., antibodies or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules (e.g., minor or insignificant amounts of impurity may remain) or to an absence of water, buffers, or salts or to components of a pharmaceutical formulation that includes the antigen-binding proteins (e.g., antibodies or antigen-binding fragments).

The present disclosure includes antigen-binding proteins, e.g., antibodies or antigen-binding fragments, that bind to the same epitope as an antigen-binding protein described herein. In some embodiments, provided is an antigen-binding protein that binds specifically to transferrin receptor or an antigenic-fragment thereof or variant thereof which binds to one or more epitopes of hTfR selected from: (a) an epitope comprising the sequence LLNE (SEQ ID NO: 796) and/or an epitope comprising the sequence TYKEL (SEQ ID NO: 706); (b) an epitope comprising the sequence DSTDFTGT (SEQ ID NO: 797) and/or an epitope comprising the sequence VKHPVTGQF (SEQ ID NO: 798) and/or an epitope comprising the sequence IERIPEL (SEQ ID NO: 799); (c) an epitope comprising the sequence LNENSYVPREAGSQKDEN (SEQ ID NO: 800); (d) an epitope comprising the sequence FEDL (SEQ ID NO: 718); (e) an epitope comprising the sequence IVDKNGRL (SEQ ID NO: 801); (f) an epitope comprising the sequence IVDKNGRLVY (SEQ ID NO: 802); (g) an epitope comprising the sequence DQTKF (SEQ ID NO: 803); (h) an epitope comprising the sequence LVENPGGY (SEQ ID NO: 804) and/or an epitope comprising the sequence PIVNAELSF (SEQ ID NO: 805) and/or an epitope comprising the sequence PYLGTTMDT (SEQ ID NO: 806); (i) an epitope comprising the sequence LLNENSYVPREAGSQKDENLAL (SEQ ID NO: 704) and/or an epitope comprising the sequence IYMDQTKFPIVNAEL (SEQ ID NO: 705) and/or an epitope comprising the sequence TYKEL (SEQ ID NO: 706); (j) an epitope comprising the sequence KRKLSEKLDSTDFTGTIKL (SEQ ID NO: 707) and/or an epitope comprising the sequence YTLIEKTMQNVKHPVTGQFL (SEQ ID NO: 708) and/or an epitope comprising the sequence LIERIPELNKVARAAAE (SEQ ID NO: 709); (k) an epitope comprising the sequence LNENSYVPREAGSQKDENL (SEQ ID NO: 710); (1) an epitope comprising the sequence GTKKDFEDL (SEQ ID NO: 711); (m) an epitope comprising the sequence SVIIVDKNGRLVYLVENPGGYVAYSK (SEQ ID NO: 712); (n) an epitope comprising the sequence LLNENSYVPREAGSQKDEN (SEQ ID NO: 713) and/or an epitope comprising the sequence DQTKFPIVNAEL (SEQ ID NO: 714) and/or an epitope comprising the sequence TYKELIERIPELNK (SEQ ID NO: 715); (o) an epitope comprising the sequence LLNENSYVPREAGSQKDEN (SEQ ID NO: 713) and/or an epitope comprising the sequence TYKELIERIPELNK (SEQ ID NO: 715); (p) an epitope comprising the sequence SVIIVDKNGRLVYLVENPGGYVAY (SEQ ID NO: 716); (q) an epitope comprising the sequence IYMDQTKFPIVNAEL (SEQ ID NO: 705) and/or an epitope comprising the sequence FGNMEGDCPSDWKTDSTCRM (SEQ ID NO: 717); (r) an epitope comprising the sequence LLNENSYVPREAGSQKDENLAL (SEQ ID NO: 704) and/or an epitope comprising the sequence LVENPGGYVAYSKAATVTGKL (SEQ ID NO: 719) and/or an epitope comprising the sequence IYMDQTKFPIVNAELSF (SEQ ID NO: 720) and/or an epitope comprising the sequence ISRAAAEKL (SEQ ID NO: 721) and/or an epitope comprising the sequence VTSESKNVKLTVSNVLKE (SEQ ID NO: 722) and/or an epitope comprising the sequence FCEDTDYPYLGTTMDT (SEQ ID NO: 723); (s) an epitope comprised within or overlapping with the sequence LLNENSYVPREAGSQKDENLAL (SEQ ID NO: 704) and/or an epitope comprised within or overlapping with the sequence IYMDQTKFPIVNAEL (SEQ ID NO: 705) and/or an epitope comprised within or overlapping with the sequence TYKEL (SEQ ID NO: 706); (t) an epitope comprised within or overlapping with the sequence KRKLSEKLDSTDFTGTIKL (SEQ ID NO: 707) and/or an epitope comprised within or overlapping with the sequence YTLIEKTMQNVKHPVTGQFL (SEQ ID NO: 708) and/or an epitope comprised within or overlapping with the sequence LIERIPELNKVARAAAE (SEQ ID NO: 709); (u) an epitope comprised within or overlapping with the sequence LNENSYVPREAGSQKDENL (SEQ ID NO: 710); (v) an epitope comprised within or overlapping with the sequence GTKKDFEDL (SEQ ID NO: 711); (w) an epitope comprised within or overlapping with the sequence SVIIVDKNGRLVYLVENPGGYVAYSK (SEQ ID NO: 712); (x) an epitope comprised within or overlapping with the sequence LLNENSYVPREAGSQKDEN (SEQ ID NO: 713) and/or an epitope comprised within or overlapping with the sequence DQTKFPIVNAEL (SEQ ID NO: 714) and/or an epitope comprised within or overlapping with the sequence TYKELIERIPELNK (SEQ ID NO: 715); (y) an epitope comprised within or overlapping with the sequence LLNENSYVPREAGSQKDEN (SEQ ID NO: 713) and/or an epitope comprised within or overlapping with the sequence TYKELIERIPELNK (SEQ ID NO: 715); (z) an epitope comprised within or overlapping with the sequence SVIIVDKNGRLVYLVENPGGYVAY (SEQ ID NO: 716); (aa) an epitope comprised within or overlapping with the sequence IYMDQTKFPIVNAEL (SEQ ID NO: 705) and/or an epitope comprised within or overlapping with the sequence FGNMEGDCPSDWKTDSTCRM (SEQ ID NO: 717); and (bb) an epitope comprised within or overlapping with the sequence LLNENSYVPREAGSQKDENLAL (SEQ ID NO: 704) and/or an epitope comprised within or overlapping with the sequence LVENPGGYVAYSKAATVTGKL (SEQ ID NO: 719) and/or an epitope comprised within or overlapping with the sequence IYMDQTKFPIVNAELSF (SEQ ID NO: 720) and/or an epitope comprised within or overlapping with the sequence ISRAAAEKL (SEQ ID NO: 721) and/or an epitope comprised within or overlapping with the sequence VTSESKNVKLTVSNVLKE (SEQ ID NO: 722) and/or an epitope comprised within or overlapping with the sequence FCEDTDYPYLGTTMDT (SEQ ID NO: 723). In some embodiments, provided is an antigen-binding protein, wherein the antigen binding protein comprises an antibody or antigen-binding fragment thereof which binds to one or more epitopes of hTfR selected from: (a) an epitope consisting of the sequence LLNE (SEQ ID NO: 796) and/or an epitope consisting of the sequence TYKEL (SEQ ID NO: 706); (b) an epitope consisting of the sequence DSTDFTGT (SEQ ID NO: 797) and/or an epitope consisting of the sequence VKHPVTGQF (SEQ ID NO: 798) and/or an epitope consisting of the sequence IERIPEL (SEQ ID NO: 799); (c) an epitope consisting of the sequence LNENSYVPREAGSQKDEN (SEQ ID NO: 800); (d) an epitope consisting of the sequence FEDL (SEQ ID NO: 718); (e) an epitope consisting of the sequence IVDKNGRL (SEQ ID NO: 801); (f) an epitope consisting of the sequence IVDKNGRLVY (SEQ ID NO: 802); (g) an epitope consisting of the sequence DQTKF (SEQ ID NO: 803); (h) an epitope consisting of the sequence LVENPGGY (SEQ ID NO: 804) and/or an epitope consisting of the sequence PIVNAELSF (SEQ ID NO: 805) and/or an epitope consisting of the sequence PYLGTTMDT (SEQ ID NO: 806); (i) an epitope consisting of the sequence LLNENSYVPREAGSQKDENLAL (SEQ ID NO: 704) and/or an epitope consisting of the sequence IYMDQTKFPIVNAEL (SEQ ID NO: 705) and/or an epitope consisting of the sequence TYKEL (SEQ ID NO: 706); (j) an epitope consisting of the sequence KRKLSEKLDSTDFTGTIKL (SEQ ID NO: 707) and/or an epitope consisting of the sequence YTLIEKTMQNVKHPVTGQFL (SEQ ID NO: 708) and/or an epitope consisting of the sequence LIERIPELNKVARAAAE (SEQ ID NO: 709); (k) an epitope consisting of the sequence LNENSYVPREAGSQKDENL (SEQ ID NO: 710); (1) an epitope consisting of the sequence GTKKDFEDL (SEQ ID NO: 711); (m) an epitope consisting of the sequence SVIIVDKNGRLVYLVENPGGYVAYSK (SEQ ID NO: 712); (n) an epitope consisting of the sequence LLNENSYVPREAGSQKDEN (SEQ ID NO: 713) and/or an epitope consisting of the sequence DQTKFPIVNAEL (SEQ ID NO: 714) and/or an epitope consisting of the sequence TYKELIERIPELNK (SEQ ID NO: 715); (o) an epitope consisting of the sequence LLNENSYVPREAGSQKDEN (SEQ ID NO: 713) and/or an epitope consisting of the sequence TYKELIERIPELNK (SEQ ID NO: 715); (p) an epitope consisting of the sequence SVIIVDKNGRLVYLVENPGGYVAY (SEQ ID NO: 716); (q) an epitope consisting of the sequence IYMDQTKFPIVNAEL (SEQ ID NO: 705) and/or an epitope consisting of the sequence FGNMEGDCPSDWKTDSTCRM (SEQ ID NO: 717); and (r) an epitope consisting of the sequence LLNENSYVPREAGSQKDENLAL (SEQ ID NO: 704) and/or an epitope consisting of the sequence LVENPGGYVAYSKAATVTGKL (SEQ ID NO: 719) and/or an epitope consisting of the sequence IYMDQTKFPIVNAELSF (SEQ ID NO: 720) and/or an epitope consisting of the sequence ISRAAAEKL (SEQ ID NO: 721) and/or an epitope consisting of the sequence VTSESKNVKLTVSNVLKE (SEQ ID NO: 722) and/or an epitope consisting of the sequence FCEDTDYPYLGTTMDT (SEQ ID NO: 723).

An antigen is a molecule, such as a peptide (e.g., TfR or a fragment thereof (an antigenic fragment)), to which, for example, an antibody or antigen-binding fragment thereof binds. The specific region on an antigen that an antibody recognizes and binds to is called the epitope. Antigen-binding proteins (e.g., antibodies) described herein that specifically bind to such antigens are part of the present disclosure.

The term “epitope” refers to an antigenic determinant (e.g., on TfR) that interacts with a specific antigen-binding site of an antigen-binding protein, e.g., a variable region of an antibody, known as a paratope. A single antigen may have more than one epitope. Thus, different antibodies may bind to different areas on an antigen and may have different biological effects. The term “epitope” may also refer to a site on an antigen to which B and/or T cells respond and/or to a region of an antigen that is bound by an antibody. Epitopes may be defined as structural or functional. Functional epitopes are generally a subset of the structural epitopes and have those residues that directly contribute to the affinity of the interaction. Epitopes may be linear or conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes to which antigen-binding proteins described herein bind may be included in fragments of TfR, for example, the extracellular domain thereof. Antigen-binding proteins (e.g., antibodies) described herein that bind to such epitopes are part of the present disclosure.

Methods for determining the epitope of an antigen-binding protein, e.g., antibody or fragment or polypeptide, include alanine scanning mutational analysis, peptide blot analysis (Reineke (2004) Methods Mol. Biol. 248: 443-63), peptide cleavage analysis, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antigen-binding protein (e.g., antibody or fragment or polypeptide) interacts is hydrogen/deuterium exchange detected by mass spectrometry. See, e.g., Ehring (1999) Analytical Biochemistry 267: 252-259; Engen and Smith (2001) Anal. Chem. 73: 256A-265A.

The present disclosure includes antigen-binding proteins that compete for binding to a TfR epitope as discussed herein, with an antigen-binding protein described herein. The term “competes” as used herein, refers to an antigen-binding protein (e.g., antibody or antigen-binding fragment thereof) that binds to an antigen (e.g., TfR) and inhibits or blocks the binding of another antigen-binding protein (e.g., antibody or antigen-binding fragment thereof) to the antigen. Unless otherwise stated, the term also includes competition between two antigen-binding proteins e.g., antibodies, in both orientations, i.e., a first antibody that binds antigen and blocks binding by a second antibody and vice versa. Thus, in an embodiment, competition occurs in one such orientation. In certain embodiments, the first antigen-binding protein (e.g., antibody) and second antigen-binding protein (e.g., antibody) may bind to the same epitope. Alternatively, the first and second antigen-binding proteins (e.g., antibodies) may bind to different, but, for example, overlapping or non-overlapping epitopes, wherein binding of one inhibits or blocks the binding of the second antibody, e.g., via steric hindrance. Competition between antigen-binding proteins (e.g., antibodies) may be measured by methods known in the art, for example, by a real-time, label-free bio-layer interferometry assay. Also, binding competition between TfR-binding proteins (e.g., monoclonal antibodies (mAbs)) can be determined using a real time, label-free bio-layer interferometry assay on an Octet RED384 biosensor (Pall ForteBio Corp.).

Typically, an antibody or antigen-binding fragment described herein which is modified in some way retains the ability to specifically bind to TfR, e.g., retains at least 10% of its TfR binding activity (when compared to the parental antibody) when that activity is expressed on a molar basis. Preferably, an antibody or antigen-binding fragment described herein retains at least 20%, 50%, 70%, 80%, 90%, 95% or 100% or more of the TfR binding affinity as the parental antibody. It is also intended that an antibody or antigen-binding fragment described herein may include conservative or non-conservative amino acid substitutions (referred to as “conservative variants” or “function conserved variants” of the antibody) that do not substantially alter its biologic activity.

An anti-TfR antigen-binding protein provided herein may be a monoclonal antibody or an antigen-binding fragment of a monoclonal antibody which may be tethered to a GAA polypeptide. Provided herein are monoclonal anti-TfR antigen-binding proteins, e.g., antibodies and antigen-binding fragments thereof, as well as monoclonal compositions comprising a plurality of isolated monoclonal antigen-binding proteins. The term “monoclonal antibody” or “mAb,” as used herein, refers to a member of a population of substantially homogeneous antibodies, i.e., the antibody molecules comprising the population are identical in amino acid sequence except for possible naturally occurring mutations that may be present in minor amounts. A “plurality” of such monoclonal antibodies and fragments in a composition refers to a concentration of identical (i.e., as discussed above, in amino acid sequence except for possible naturally occurring mutations that may be present in minor amounts) antibodies and fragments which is above that which would normally occur in nature, e.g., in the blood of a host organism such as a mouse or a human.

In an embodiment, an anti-TfR antigen-binding protein, e.g., antibody or antigen-binding fragment (which may be tethered to a Payload) comprises a heavy chain constant domain, e.g., of the type IgA (e.g., IgA1 or IgA2), IgD, IgE, IgG (e.g., IgG1, IgG2, IgG3 and IgG4) or IgM. In an embodiment, an antigen-binding protein, e.g., antibody or antigen-binding fragment, comprises a light chain constant domain, e.g., of the type kappa or lambda. In an embodiment, a V_{H} as set forth herein is linked to a human heavy chain constant domain (e.g., IgG) and a V_{L} as set forth herein is linked to a human light chain constant domain (e.g., kappa). The present disclosure includes antigen-binding proteins comprising the variable domains set forth herein, which are linked to a heavy and/or light chain constant domain, e.g., as set forth herein.

Included herein are human anti-TfR antigen-binding proteins which may be tethered to a GAA polypeptide. The term “human” antigen-binding protein, such as an antibody or antigen-binding fragment, as used herein, includes antibodies and fragments having variable and constant regions derived from human germline immunoglobulin sequences whether in a human cell or grafted into a non-human cell, e.g., a mouse cell. See, e.g., U.S. Pat. No. 8,502,018, 6,596,541 or 5,789,215. The anti-TfR human mAbs provided herein may include amino acid residues not encoded by human germline immunoglobulin sequences (e.g., mutations introduced by random or site-specific mutagenesis in vitro or by somatic mutation in vivo), for example, in the CDRs and in particular CDR3. However, the term “human antibody,” as used herein, is not intended to include mAbs in which CDR sequences derived from the germline of another mammalian species (e.g., mouse) have been grafted onto human FR sequences. The term includes antibodies recombinantly produced in a non-human mammal or in cells of a non-human mammal. The term is not intended to include natural antibodies directly isolated from a human subject. The present disclosure includes human antigen-binding proteins (e.g., antibodies or antigen-binding fragments thereof described herein).

Also included herein are anti-TfR chimeric antigen-binding proteins, e.g., antibodies and antigen-binding fragments thereof (which may be tethered to a GAA polypeptide), and methods of use thereof. As used herein, a “chimeric antibody” is an antibody having the variable domain from a first antibody and the constant domain from a second antibody, where the first and second antibodies are from different species. (see, e.g., U.S. Pat. No. 4,816,567; and Morrison et al., (1984) Proc. Natl. Acad. Sci. USA 81: 6851-6855). The present disclosure includes chimeric antibodies comprising the variable domains which are set forth herein and a non-human constant domain.

The term “recombinant” anti-TfR antigen-binding proteins, such as antibodies or antigen-binding fragments thereof (which may be tethered to a GAA polypeptide), refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a cell (e.g., CHO cells) such as a cellular expression system or isolated from a recombinant combinatorial human antibody library. The present disclosure includes recombinant antigen-binding proteins, such as antibodies and antigen-binding fragments as set forth herein.

An antigen-binding fragment of an antibody will, in an embodiment, comprise less than a full antibody but still binds specifically to antigen, e.g., TfR, e.g., including at least one variable domain. The variable domain may be of any size or amino acid composition and will generally comprise at least one (e.g., 3) CDR(s), which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} and/or V_{L} domain which are bound non-covalently.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody described herein include: (i) V_{H}-CH1; (ii) V_{H}-CH2; (iii) V_{H}-CH3; (iv) V_{H}-CH1-CH2; (v) V_{H}-CH1-CH2-CH3; (vi) V_{H}-CH2-CH3; (vii) V_{H}-CL; (viii) V_{L}-CH1; (ix) V_{L}-CH2; (x) V_{L}-CH3; (xi) V_{L}-CH1-CH2; (xii) V_{L}-CH1-CH2-CH3; (xiii) V_{L}-CH2-CH3; and (xiv) V_{L}-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody described herein may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)). The present disclosure includes an antigen-binding fragment of an antigen-binding protein such as an antibody set forth herein.

Antigen-binding proteins (e.g., antibodies and antigen-binding fragments) may be monospecific or multi-specific (e.g., bispecific). Multispecific antigen-binding proteins are discussed further herein. The present disclosure includes monospecific as well as multispecific (e.g., bispecific) antigen-binding fragments comprising one or more variable domains from an antigen-binding protein that is specifically set forth herein.

A “variant” of a polypeptide, such as an immunoglobulin chain, refers to a polypeptide comprising an amino acid sequence that is at least about 70-99.9% (e.g., at least 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein (e.g., any of SEQ ID NOS: 171-174; 680; 176-179; 181-184; 681; 186-189; 191-194; 682; 196-199; 201-204; 206-209; 683; 211-214; 216-219; 684; 221-224; 685; 226-229; 686; 231-234; 687; 236-239; 688; 241-244; 689; 246-249; 690; 251-254; 256-259; 261-264; 691; 266-269; 271-274; 276-279; 281-284; 692; 286-289; 693; 291-294; 296-299; 301-304; 306-309; 311-314; 694; 316-319; 695; 321-324; 326-329; 331-334; 696; 336-339; 341-344; 346-349; 351-354; 697; 356-359; 698; 361-364; 699; 366-369; 371-374; 700; 376-379; 381-384; 386-389; 391-394; 396-399; 401-404; 406-409; 411-414; 416-419; 421-424; 701; 426-429; 431-434; 436-439; 441-444; 446-449; 451-454; 456-459; 461-464; 466-469; 471-474; 702; 476-479; 481-484; 486-489; 703; 492-523, 540-609, 632-638, 737, or 739); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment) and/or comprising the amino acid sequence but having one or more (e.g., 1, 2, 3, 4, 5, 6, 7, 8, 9 or 10) mutations (e.g., point mutation, insertion, truncation, and/or deletion).

Moreover, a variant of a polypeptide may include a polypeptide such as an immunoglobulin chain which may include the amino acid sequence of the reference polypeptide whose amino acid sequence is specifically set forth herein but for one or more (e.g., 1, 2, 3, 4, 5, 6, 7, 8, 9 or 10) mutations, e.g., one or more missense mutations (e.g., conservative substitutions), non-sense mutations, deletions, or insertions. For example, the present disclosure includes TfR-binding proteins which include an immunoglobulin light chain (or V_{L}) variant comprising the amino acid sequence set forth in SEQ ID NO: 176, 186, 196, 206, 683, 216, 684, 226, 686, 236, 688, 246, 690, 256, 266, 276, 286, 693, 296, 306, 316, 695, 326, 336, 346, 356, 698, 366, 376, 386, 396, 406, 416, 426, 436, 446, 456, 466, 476, 632, 486, or 703 but having one or more of such mutations and/or an immunoglobulin heavy chain (or V_{H}) variant comprising the amino acid sequence set forth in SEQ ID NO: 171, 680, 181, 681, 191, 682, 201, 211, 221, 685, 231, 687, 241, 689, 251, 261, 691, 271, 281, 692, 291, 301, 311, 694, 321, 331, 696, 341, 351, 697, 361, 699, 371, 700, 381, 391, 401, 411, 421, 701, 431, 441, 451, 461, 471, 702, or 481 but having one or more of such mutations. In an embodiment, a TfR-binding protein includes an immunoglobulin light chain variant comprising CDR-L1, CDR-L2 and CDR-L3 wherein one or more (e.g., 1 or 2 or 3) of such CDRs has one or more of such mutations (e.g., conservative substitutions) and/or an immunoglobulin heavy chain variant comprising CDR-H1, CDR-H2 and CDR-H3 wherein one or more (e.g., 1 or 2 or 3) of such CDRs has one or more of such mutations (e.g., conservative substitutions).

The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

A “conservatively modified variant” or a “conservative substitution”, e.g., of an immunoglobulin chain set forth herein, refers to a variant wherein there is one or more substitutions of amino acids in a polypeptide with other amino acids having similar characteristics (e.g., charge, side-chain size, hydrophobicity/hydrophilicity, backbone conformation and rigidity, etc.). Such changes can frequently be made without significantly disrupting the biological activity of the antibody or fragment. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4^{th} Ed.)). In addition, substitutions of structurally or functionally similar amino acids are less likely to significantly disrupt biological activity. The present disclosure includes TfR-binding proteins comprising such conservatively modified variant immunoglobulin chains.

Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Alternatively, a conservative replacement is any change having a positive value in the PAM250 log-likelihood matrix disclosed in Gonnet et al. (1992) Science 256: 1443-45.

Antibodies and antigen-binding fragments described herein comprise immunoglobulin chains including the amino acid sequences specifically set forth herein (and variants thereof) as well as cellular and in vitro post-translational modifications to the antibody or fragment. For example, the present disclosure includes antibodies and antigen-binding fragments thereof that specifically bind to TfR comprising heavy and/or light chain amino acid sequences set forth herein as well as antibodies and fragments wherein one or more asparagine, serine and/or threonine residues is glycosylated, one or more asparagine residues is deamidated, one or more residues (e.g., Met, Trp and/or His) is oxidized, the N-terminal glutamine is pyroglutamate (pyroE) and/or the C-terminal lysine or other amino acid is missing.

In an embodiment, an anti-hTfR:Payload or anti-hTfR:Payload (e.g., in scFv, Fab, antibody or antigen-binding fragment thereof format), e.g., wherein the Payload is human GAA, exhibits one or more of the following characteristics:
Affinity (K_{D}) for binding to human TfR at 25° C. in surface plasmon resonance format of about 41 nM or a higher affinity (e.g., about 1 or 0.1 nM or about 0.18 to about 1.2 nM, or higher);
Affinity (K_{D}) for binding to monkey TfR at 25° C. in surface plasmon resonance format of about 0 nM (no detectable binding) or a higher affinity (e.g., about 20 nM or higher);
Ratio of K_{D} for binding to monkey TfR/human TfR at 25° C. in surface plasmon resonance format of from 0 to 278 (e.g., about 17 or 18);
Blocks about 3, 5, 10 or 13% hTfR (e.g., Hmm-hTFRC such as REGN2431) binding to Human Holo-Tf when in Fab format (IgG1), e.g., no more than about 45% blocking;
Blocks about 6, 8, 10 or 13% hTfR (e.g., Hmm-hTFRC such as REGN2431) binding to Human Holo-Tf when in scFv (V_{K}-V_{H}) format, e.g., no more than about 45% blocking;
Blocks about 11, 17, 23 or 26% hTfR (e.g., Hmm-hTFRC such as REGN2431) binding to Human Holo-Tf when in scFv (V_{H}-V_{L}) format, e.g., no more than about 45% blocking;
Exhibits a ratio of about 1 or greater; 0.67 or greater; 1.08 or greater; 0.91 or greater; 0.65 or greater; 0.55 or greater; 0.50 or greater; 0.27 or greater; 0.72 or greater; 1.05 or greater; 0.49 or greater; 0.29 or greater; 1.29 or greater; 1.72 or greater; 1.79 or greater; 3.08 or greater; 1.24 or greater; 0.59 or greater; or 0.47 or greater (or about 1-2 or greater) mature hGAA protein in brain (normalized to that of positive control 8D3:GAA scFv) in mice (e.g., Tfrc^{hum/hum} knock-in mice) administered the molecule via HDD, when in anti-hTfR scFv:hGAA format; or delivers mature human GAA protein to the brain of humans administered said scFv:hGAA molecule;
Exhibits a ratio of about 0.44, 0.05, 1.13 or 0.60 (about 0.1-1.2) mature hGAA protein in brain parenchyma (normalized to that of positive control 8D3:GAA scFv) in mice (e.g., Tfrc^{hum/hum} knock-in mice) administered the molecule via HDD, when in anti-hTfR scFv:hGAA format; or delivers mature human GAA protein to the brain parenchyma of humans administered said scFv:hGAA molecule;
Exhibits a ratio of about 0.67, 1.80, 1.78 or 7.74 (about 1-2) mature hGAA protein in quadriceps (normalized to that of positive control 8D3:GAA scFv) in mice (e.g., Tfrc^{hum/hum} knock-in mice) administered the molecule via HDD, when in anti-hTfR scFv:hGAA format; or delivers mature human GAA protein to the quadricep or other muscle tissue of humans administered said scFv:hGAA molecule;
Exhibits a ratio of about 0.94, 0.49, 0.61 or 1.90 (about 0.1-1.2) mature hGAA protein in brain parenchyma (normalized to that of positive control 8D3:GAA scFv) in mice (e.g., Tfrc^{hum} knock-in mice) administered the molecule via AAV8 liver depot, when in anti-hTfR scFv:hGAA format; or delivers mature human GAA protein to the brain parenchyma of humans administered said scFv:hGAA molecule via viral, e.g., AAV, liver depot or parenterally delivered in protein scFv:hGAA fusion format;
Delivers mature hGAA protein to serum, liver, cerebrum, cerebellum, spinal cord, heart and/or quadricep in mice (e.g., Tfrc^{hum} knock-in mice) administered the molecule via AAV8 liver depot, when in anti-hTfR scFv:hGAA format; or delivers mature human GAA protein to the serum, liver, cerebrum, cerebellum, spinal cord, heart and/or quadricep of humans administered said scFv:hGAA molecule via viral, e.g., AAV, liver depot or parenterally delivered in protein scFv:hGAA fusion format;
Reduces glycogen stored in cerebrum, cerebellum, spinal cord, heart and/or quadricep in mice (e.g., Tfrc^{hum} knock-in mice) administered the molecule via AAV8 liver depot, when in anti-hTfR scFv:hGAA format; e.g., by at least 75% to greater than 95% or greater than 99%; or reduces glycogen stored in cerebrum, cerebellum, spinal cord, heart and/or quadricep of humans administered said scFv:hGAA molecule via viral, e.g., AAV, liver depot, or parenterally delivered in protein scFv:hGAA fusion format;
Reduces glycogen levels in tissues (e.g., cerebellum) of Gaa^{-/-}/Tfrc^{hum} mice treated with liver-depot AAV8 anti-hTFRC scfv:hGAA (e.g., 4e11 vg/kg AAV8) by at least about 90% (e.g., about 95% or more) relative to untreated Gaa^{-/-}/Tfrc^{hum} mice;
Reduces glycogen levels in tissues (e.g., quadricep) of Gaa^{-/-}/Tfrc^{hum} mice treated with liver-depot AAV8 anti-hTFRC scfv:hGAA (e.g., 4e11 vg/kg AAV8) by at least about 89% (e.g., about 90% or 91% or more) relative to untreated Gaa^{-/-}/Tfrc^{hum} mice; or of humans treated with the fusion, e.g., by parenteral deliver of the fusion protein;
Does not cause abnormal iron homeostasis when administered (e.g., by HDD or AAV8 episomal liver depot) to Tfrc^{hum} mice; e.g., wherein the mice maintain normal serum, heart, liver and/or spleen iron levels, normal total iron-binding capacity (TIBC), and/or normal hepcidin levels); or when administered to humans, e.g., by parenteral deliver of the fusion protein;
When chromosomally inserted (e.g., into the albumin gene locus) or delivered episomally to a subject (e.g., to a human or Gaa^{-/-}/Tfrc^{hum/hum} mouse), for example, in an AAV8 vector, DNA encoding the fusion causes expression of mature human GAA to serum, liver, cerebrum and/or quadricep; and/or
When chromosomally inserted (e.g., into the albumin gene locus) or delivered episomally (e.g., to a human or Gaa^{-/-}/Tfrc^{hum/hum} mouse), for example, in an AAV8 vector, DNA encoding the fusion reduces glycogen levels in the cerebrum and/or quadricep.
Tfrc^{hum} or Tfrc^{hum/hum} are homozygous knock-in mice.

The amino acid sequences of domains in anti-human transferrin receptor antigen-binding proteins of fusions disclosed herein are summarized below in Table 2. For example, anti-human transferrin receptor 1 antibodies and antigen-binding fragments thereof (e.g., scFvs and Fabs) comprising the HCVR and LCVR of the molecules in Table 2; or comprising the CDRs thereof, fused to a GAA polypeptide, are disclosed herein. In a specific example, the anti-human transferrin receptor 1 antibodies and antigen-binding fragments thereof (e.g., scFvs and Fabs) comprise the HCVR and LCVR of or comprise the CDRs of #23 or #25 in Table 2. In a specific example, the anti-human transferrin receptor 1 antibodies and antigen-binding fragments thereof (e.g., scFvs and Fabs) comprise the HCVR and LCVR of or comprise the CDRs of #23 in Table 2. In a specific example, the anti-human transferrin receptor 1 antibodies and antigen-binding fragments thereof (e.g., scFvs and Fabs) comprise the HCVR and LCVR of or comprise the CDRs of #25 in Table 2.

TABLE 2
Domains in Anti-hTfR Antibodies, Antigen-binding Fragments
(e.g., Fabs) or scFv Molecules in Fusion Proteins.

| | anti- | HC- | | | | | LC- | | | | |
|---|---|---|---|---|---|---|---|---|---|---|---|
| | hTfR | VR | HC-VR | | | | VR | LC-VR | | | |
| # | Molecule | NT | AA | HCDR1 | HCDR2 | HCDR3 | NT | AA | LCDR1 | LCDR2 | LCDR3 |
| 1 | 31874B | 170 | 171 or 680 | 172 | 173 | 174 | 175 | 176 | 177 | 178 | 179 |
| 2 | 31863B | 180 | 181 or 681 | 182 | 183 | 184 | 185 | 186 | 187 | 188 | 189 |
| 3 | 69348 | 190 | 191 or 682 | 192 | 193 | 194 | 195 | 196 | 197 | 198 | 199 |
| 4 | 69340 | 200 | 201 | 202 | 203 | 204 | 205 | 206 or 683 | 207 | 208 | 209 |
| 5 | 69331 | 210 | 211 | 212 | 213 | 214 | 215 | 216 or 684 | 217 | 218 | 219 |
| 6 | 69332 | 220 | 221 or 685 | 222 | 223 | 224 | 225 | 226 or 686 | 227 | 228 | 229 |
| 7 | 69326 | 230 | 231 or 687 | 232 | 233 | 234 | 235 | 236 or 688 | 237 | 238 | 239 |
| 8 | 69329 | 240 | 241 or 689 | 242 | 243 | 244 | 245 | 246 or 690 | 247 | 248 | 249 |
| 9 | 69323 | 250 | 251 | 252 | 253 | 254 | 255 | 256 | 257 | 258 | 259 |
| 10 | 69305 | 260 | 261 or 691 | 262 | 263 | 264 | 265 | 266 | 267 | 268 | 269 |
| 11 | 69307 | 270 | 271 | 272 | 273 | 274 | 275 | 276 | 277 | 278 | 279 |
| 12 | 12795B | 280 | 281 or 692 | 282 | 283 | 284 | 285 | 286 or 693 | 287 | 288 | 289 |
| 13 | 12798B | 290 | 291 | 292 | 293 | 294 | 295 | 296 | 297 | 298 | 299 |
| 14 | 12799B | 300 | 301 | 302 | 303 | 304 | 305 | 306 | 307 | 308 | 309 |
| 15 | 12801B | 310 | 311 or 694 | 312 | 313 | 314 | 315 | 316 or 695 | 317 | 318 | 319 |
| 16 | 12802B | 320 | 321 | 322 | 323 | 324 | 325 | 326 | 327 | 328 | 329 |
| 17 | 12808B | 330 | 331 or 696 | 332 | 333 | 334 | 335 | 336 | 337 | 338 | 339 |
| 18 | 12812B | 340 | 341 | 342 | 343 | 344 | 345 | 346 | 347 | 348 | 349 |
| 19 | 12816B | 350 | 351 or 697 | 352 | 353 | 354 | 355 | 356 or 698 | 357 | 358 | 359 |
| 20 | 12833B | 360 | 361 or 699 | 362 | 363 | 364 | 365 | 366 | 367 | 368 | 369 |
| 21 | 12834B | 370 | 371 or 700 | 372 | 373 | 374 | 375 | 376 | 377 | 378 | 379 |
| 22 | 12835B | 380 | 381 | 382 | 383 | 384 | 385 | 386 | 387 | 388 | 389 |
| 23 | 12847B | 390 | 391 | 392 | 393 | 394 | 395 | 396 | 397 | 398 | 399 |
| 24 | 12848B | 400 | 401 | 402 | 403 | 404 | 405 | 406 | 407 | 408 | 409 |
| 25 | 12843B | 410 | 411 | 412 | 413 | 414 | 415 | 416 | 417 | 418 | 419 |
| 26 | 12844B | 420 | 421 or 701 | 422 | 423 | 424 | 425 | 426 | 427 | 428 | 429 |
| 27 | 12845B | 430 | 431 | 432 | 433 | 434 | 435 | 436 | 437 | 438 | 439 |
| 28 | 12839B | 440 | 441 | 442 | 443 | 444 | 445 | 446 | 447 | 448 | 449 |
| 29 | 12841B | 450 | 451 | 452 | 453 | 454 | 455 | 456 | 457 | 458 | 459 |
| 30 | 12850B | 460 | 461 | 462 | 463 | 464 | 465 | 466 | 467 | 468 | 469 |
| 31 | 69261 | 470 | 471 or 702 | 472 | 473 | 474 | 475 | 476 or 632 | 477 | 478 | 479 |
| 32 | 69263 | 480 | 481 | 482 | 483 | 484 | 485 | 486 or 703 | 487 | 488 | 489 |

31874B
HCVR (V_{H}) Nucleotide Sequence
(SEQ ID NO: 170)
GAGGTGCAGCTGGTGGAGTCTGGGGGAGGCTTGGTACAGCCTGGGGGGTCCCTGAGACTCTCCTGTGCAGCCTCTGG
ATTCGCCTTTAGCAGCTATGCCATGACCTGGGTCCGACAGGCTCCAGGGAAGGGGCTGGAGTGGGTCTCAGTTATCA
GTGGTACTGGTGGTAGTACATACTACGCAGACTCCGTGAAGGGCCGGTTCACCATCTCCAGAGACAATTCCAAGAAC
ACGCTGTATCTACAAATGAACAGCCTGAGAGCCGAGGACACGGCCGTATATTACTGTGCGAAAGGGGGAGCAGCTCG
TAGAATGGAATACTTCCAGTACTGGGGCCAGGGCACCCTGGTCACCGTCTCCTCA
HCVR (V_{H}) Amino Acid Sequence
(SEQ ID NO: 171)
EVQLVESGGGLVQPGGSLRLSCAASGFAFSSYAMTWVRQAPGKGLEWVSVISGTGGSTYYADSVKGRFTISRDNSKN
TLYLQMNSLRAEDTAVYYCAKGGAARRMEYFQYWGQGTLVTVSS
or
(SEQ ID NO: 680)
EVQLVESGGGLVQPGGSLRLSCAASGFAFSSYAMTWVRQAPGKGLEWVSVISGTGGSTYYADSVKGRFTISRDNSKN
TLYLQMNSLRAEDTAVYYCAKGGAARRMEYFQYWGQGTTVTVSS
HCDR1:
(SEQ ID NO: 172)
GFAFSSYA
HCDR2:
(SEQ ID NO: 173)
ISGTGGST
HCDR3:
(SEQ ID NO: 174)
AKGGAARRMEYFQY
LCVR (V_{L}) Nucleotide Sequence
(SEQ ID NO: 175)
GACATCCAGATGACCCAGTCTCCATCCTCCCTGTCTGCATCTGTAGGAGACAGAGTCACCATCACTTGCCGGGCGAG
TCAGGGCATTAGCAATTATTTAGCCTGGTATCAGCAGAAACCAGGGAAAGTTCCTAACCTCCTTATCTATGCTGCAT
CCACTTTGCAATCAGGGGTCCCATCTCGATTCAGTGGCAGTGGATCTGGGACAGATTTCACTCTCACCATCAGCAGC
CTGCAGCCTGAAGATGTTGCAACTTATTACTGTCAAAAGTATAACAGTGCCCCTCTCACTTTCGGCGGAGGGACCAA
GGTGGAGATCAAA
LCVR (V_{L}) Amino Acid Sequence
(SEQ ID NO: 176)
DIQMTQSPSSLSASVGDRVTITCRASQGISNYLAWYQQKPGKVPNLLIYAASTLQSGVPSRFSGSGSGTDFTLTISS
LQPEDVATYYCQKYNSAPLTFGGGTKVEIK
LCDR1:
(SEQ ID NO: 177)
QGISNY
LCDR2:
(SEQ ID NO: 178)
AAS
LCDR3:
(SEQ ID NO: 179)
QKYNSAPLT
31863B
HCVR (V_{H}) Nucleotide Sequence
(SEQ ID NO: 180)
GAGGTGCAGCTGGTGGAGTCTGGGGGAGGCTTGGTACAGCCTGGGGGGTCCCTGAGACTCTCCTGTGCAGCCTCTGG
ATTCACCTTTAACAGCTATGCCATGACCTGGGTCCGCCAGGCTCCAGGGAAGGGGCTGGAGTGGGTCTCATTTATTG
GTGGTAGTACTGGTAACACATACTACGCAGGCTCCGTGAAGGGCCGGTTCACCATCTCCAGCGACAATTCCAAGAAG
ACGCTGTATCTGCAAATGAACAGCCTGAGAGCCGAGGACACGGCCGTATATTACTGTGCGAAAGGGGGAGCAGCTCG
TAGAATGGAATACTTCCAGCACTGGGGCCAGGGCACCCTGGTCACCGTCTCCTCA
HCVR (V_{H}) Amino Acid Sequence
(SEQ ID NO: 181)
EVQLVESGGGLVQPGGSLRLSCAASGFTFNSYAMTWVRQAPGKGLEWVSFIGGSTGNTYYAGSVKGRFTISSDNSKK
TLYLQMNSLRAEDTAVYYCAKGGAARRMEYFQHWGQGTLVTVSS
or
(SEQ ID NO: 681)
EVQLVESGGGLVQPGGSLRLSCAASGFTFNSYAMTWVRQAPGKGLEWVSFIGGSTGNTYYAGSVKGRFTISSDNSKK
TLYLQMNSLRAEDTAVYYCAKGGAARRMEYFQHWGQGTTVTVSS
HCDR1:
(SEQ ID NO: 182)
GFTFNSYA
HCDR2:
(SEQ ID NO: 183)
IGGSTGNT
HCDR3:
(SEQ ID NO: 184)
AKGGAARRMEYFQH
LCVR (V_{L}) Nucleotide Sequence
(SEQ ID NO: 185)
GACATCCAGATGACCCAGTCTCCATCCTCCCTGTCTGCATCTATAGGAGACAGAGTCACCATCACTTGCCGGGCGAG
TCAGGGCATTAGCAATTATTTAGCCTGGTATCAACAGAAACCAGGGAAAGTTCCTAAGCTCCTGATCTATGCTGCAT
CCACTTTGCAATCAGGGGTCCCATCTCGGTTCAGTGGCAGTGGATCTGGGACAGATTTCACTCTCACCATCAGCAGC
CTGCAGCCTGAAGATGTTGCAACTTATTACTGTCAAAACCATAACAGTGTCCCTCTCACTTTCGGCGGAGGGACCAA
GGTGGAGATCAAA
LCVR (V_{L}) Amino Acid Sequence
(SEQ ID NO: 186)
DIQMTQSPSSLSASIGDRVTITCRASQGISNYLAWYQQKPGKVPKLLIYAASTLQSGVPSRFSGSGSGTDFTLTISS
LQPEDVATYYCQNHNSVPLTFGGGTKVEIK
LCDR1:
(SEQ ID NO: 187)
QGISNY
LCDR2:
(SEQ ID NO: 188)
AAS
LCDR3:
(SEQ ID NO: 189)
QNHNSVPLT
69348
HCVR (V_{H}) Nucleotide Sequence
(SEQ ID NO: 190)
CAGGTGCAGCTGGTGGAGTCTGGGGGAGGCGTGGTCCAGCCTGGGAGGTCCCTGAGACTCTCCTGTGCAGCGTCTGG
ATTCACCTTCACTACCTATGGCATGCACTGGGTCCGCCAGGCTCCAGGCAAGGGGCTGGAGTGGGTGGCTGTTATAT
GGTATGATGGAAGTAATAAATATTATGGAGACTCCGTGAAGGGCCGATTCACCATCTCCAGAGACAATTCCAAGAAC
ACACTGTATCTGCAAATGAACAGCCTGAGAGTCGACGACACGGCTGTTTATTACTGTACGAGAACCCATGGCTATAC
CAGGTCGTCGGACGGTTTTGACTACTGGGGCCAGGGAACCCTGGTCACCGTCTCCTCA
HCVR (V_{H}) Amino Acid Sequence
(SEQ ID NO: 191)
QVQLVESGGGVVQPGRSLRLSCAASGFTFTTYGMHWVRQAPGKGLEWVAVIWYDGSNKYYGDSVKGRFTISRDNSKN
TLYLQMNSLRVDDTAVYYCTRTHGYTRSSDGFDYWGQGTLVTVSS
or
(SEQ ID NO: 682)
EVQLVESGGGVVQPGRSLRLSCAASGFTFTTYGMHWVRQAPGKGLEWVAVIWYDGSNKYYGDSVKGRFTISRDNSKN
TLYLQMNSLRVDDTAVYYCTRTHGYTRSSDGFDYWGQGTMVTVSS
HCDR1:
(SEQ ID NO: 192)
GFTFTTYG
HCDR2:
(SEQ ID NO: 193)
IWYDGSNK
HCDR3:
(SEQ ID NO: 194)
TRTHGYTRSSDGFDY
LCVR (V_{L}) Nucleotide Sequence
(SEQ ID NO: 195)
GACATCCAGATGACCCAGTCTCCATCCTCCCTGTCTGCATCTGTAGGAGACAGAGTCACCATCACTTGCCGGGCAAG
TCAGAGCATTAGAAATGTTTTAGGCTGGTTTCAGCAGAAACCAGGGAAAGCCCCTCAGCGCCTGATCTATGCTGCAT
CCAGTTTGCAAAGTGGGGTCCCATCAAGGTTCAGCGGCAGTGGATCTGGGACAGAATTCACTCTCACAATCAGCAGC
CTACAGCCTGAAGATTTTGCAACTTATTACTGTCTACAGCATAATTTTTACCCGCTCACTTTCGGCGGAGGGACCAA
GGTGGAGATCAAA
LCVR (V_{L}) Amino Acid Sequence
(SEQ ID NO: 196)
DIQMTQSPSSLSASVGDRVTITCRASQSIRNVLGWFQQKPGKAPQRLIYAASSLQSGVPSRFSGSGSGTEFTLTISS
LQPEDFATYYCLQHNFYPLTFGGGTKVEIK
LCDR1:
(SEQ ID NO: 197)
QSIRNV
LCDR2:
(SEQ ID NO: 198)
AAS
LCDR3:
(SEQ ID NO: 199)
LQHNFYPLT
69340
HCVR (V_{H}) Nucleotide Sequence
(SEQ ID NO: 200)
GAAGTGCAGCTGGTGGAGTCTGGGGGAGGCTTGGTACAGCCTGGCAGGTCCCTGAGACTCTCCTGTGCAGCCTCTGG
ATTCACCTTTGATGATAAAGCCATGCACTGGGTCCGGCAAGTTCCAGGGAAGGGCCTGGAATGGATCTCAGGTATTA
GTTGGAATAGTGGTACTATAGGCTATGCGGACTCTGTGAAGGGCCGATTCATCATCTCCAGAGACAACGCCAAGAAC
TCCCTGTATCTACAAATGAACAGTCTGAGAGCTGAGGACACGGCCTTGTATTACTGCGCAAAAGATGGAGATACCAG
TGGCTGGTACTGGTACGGTTTGGACGTCTGGGGCCAAGGGACCACGGTCACCGTCTCCTCA
HCVR (V_{H}) Amino Acid Sequence
(SEQ ID NO: 201)
EVQLVESGGGLVQPGRSLRLSCAASGFTFDDKAMHWVRQVPGKGLEWISGISWNSGTIGYADSVKGRFIISRDNAKN
SLYLQMNSLRAEDTALYYCAKDGDTSGWYWYGLDVWGQGTTVTVSS
HCDR1:
(SEQ ID NO: 202)
GFTFDDKA
HCDR2:
(SEQ ID NO: 203)
ISWNSGTI
HCDR3:
(SEQ ID NO: 204)
AKDGDTSGWYWYGLDV
LCVR (V_{L}) Nucleotide Sequence
(SEQ ID NO: 205)
GAAATTGTGTTGACACAGTCTCCTGCCACCCTGTCTTTGTCTCCAGGGGAAAGAGCCACCCTCTCCTGCAGGGCCAG
TCAGAGTGTTAGCAGCTACTTAGCCTGGTACCAACAGAAACCTGGCCAGGCTCCCAGGCTCCTCATCCATGATGTAT
CCAACAGGGCCACTGGCATCCCAGCCAGGTTCAGTGGCAGTGGGTCTGGGACAGACTTCACTCTCACCATCAGCAGT
CTAGAGCCTGAAGATTTTGTAGTTTATTACTGTCAGCAGCGTAGCGACTGGCCCATCACCTTCGGCCAAGGGACACG
ACTGGAGATTAAA
LCVR (V_{L}) Amino Acid Sequence
(SEQ ID NO: 206)
EIVLTQSPATLSLSPGERATLSCRASQSVSSYLAWYQQKPGQAPRLLIHDVSNRATGIPARFSGSGSGTDFTLTISS
LEPEDFVVYYCQQRSDWPITFGQGTRLEIK
or
(SEQ ID NO: 683)
DIVMTQSPATLSLSPGERATLSCRASQSVSSYLAWYQQKPGQAPRLLIHDVSNRATGIPARFSGSGSGTDFTLTISS
LEPEDFVVYYCQQRSDWPITFGQGTRLEIK
LCDR1:
(SEQ ID NO: 207)
QSVSSY
LCDR2:
(SEQ ID NO: 208)
DVS
LCDR3:
(SEQ ID NO: 209)
QQRSDWPIT
69331
HCVR (V_{H}) Nucleotide Sequence
(SEQ ID NO: 210)
CAGGTGCAGCTGGTGGAGTCTGGGGGAGGCGTGGTCCAGCCTGGGAGGTCCCTGAGACTCTCCTGTATAGCCTCTGG
ATTCACCTTCAGTGTCTATGGCATTCACTGGGTCCGCCAGGCTCCAGGCAAGGGGCTGGAGTGGATGGCAGTAATAT
CACATGATGGAAATATTAAACACTATGCAGACTCCGTGAAGGGCCGATTCACCATCTCCAGAGACAATTCCAAGAAC
ACGCTGTATCTTCAAATTAACAGCCTGAGAACTGAGGACACGGCTGTGTATTACTGTGCGAAAGATACCTGGAACTC
CCTTGATACTTTTGATATCTGGGGCCAAGGGACAATGGTCACCGTCTCTTCA
HCVR (V_{H}) Amino Acid Sequence
(SEQ ID NO: 211)
QVQLVESGGGVVQPGRSLRLSCIASGFTFSVYGIHWVRQAPGKGLEWMAVISHDGNIKHYADSVKGRFTISRDNSKN
TLYLQINSLRTEDTAVYYCAKDTWNSLDTFDIWGQGTMVTVSS
HCDR1:
(SEQ ID NO: 212)
GFTFSVYG
HCDR2:
(SEQ ID NO: 213)
ISHDGNIK
HCDR3:
(SEQ ID NO: 214)
AKDTWNSLDTFDI
LCVR (V_{L}) Nucleotide Sequence
(SEQ ID NO: 215)
GACATCCAGTTGACCCAGTCTCCATCCTCCCTGTCTGCATCTGTAGGAGACAGAGTCACCATCACTTGCTGGGCCAG
TCAGGGCATTAGCAGTTATTTAGCCTGGTATCAGCAAAAACCAGGGAAAGCCCCTAAGCTCCTGATCTATGCTGCAT
CCACTTTGCAAAGTGGGGTCCCATCAAGGTTCAGCGGCAGTGGATCTGGGACAGAATTCACTCTCACAATCAGCAGC
CTGCAGCCTGAAGATTTTGCAACTTATTACTGTCAACAGCTTAATAGTTACCCTCTCACTTTCGGCGGAGGGACCAA
GGTGGAGATCAAA
LCVR (V_{L}) Amino Acid Sequence
(SEQ ID NO: 216)
DIQLTQSPSSLSASVGDRVTITCWASQGISSYLAWYQQKPGKAPKLLIYAASTLQSGVPSRFSGSGSGTEFTLTISS
LQPEDFATYYCQQLNSYPLTFGGGTKVEIK
or
(SEQ ID NO: 684)
DIQMTQSPSSLSASVGDRVTITCWASQGISSYLAWYQQKPGKAPKLLIYAASTLQSGVPSRFSGSGSGTEFTLTISS
LQPEDFATYYCQQLNSYPLTFGGGTKVEIK
LCDR1:
(SEQ ID NO: 217)
QGISSY
LCDR2:
(SEQ ID NO: 218)
AAS
LCDR3:
(SEQ ID NO: 219)
QQLNSYPLT
69332
HCVR (V_{H}) Nucleotide Sequence
(SEQ ID NO: 220)
CAGGTCACCTTGAGGGAGTCTGGTCCCGCGCTGGTGAAACCCTCACAGACCCTCACACTGACCTGCACCTTCTCTGG
ATTCTCACTCAACACTTATGGGATGTTTGTGAGCTGGATCCGTCAGCCTCCAGGGAAGGCCCTAGAGTGGCTTGCAC
ACATTCATTGGGATGATGATAAATACTACAGCACATCTCTGAAGACCAGGCTCACCATCTCCAAGGACACCTCCAAA
AACCAGGTGGTCCTTACAATGACCAACATGGACCCTGTGGACACAGCCACGTATTATTGTGCACGGGGGCACAATAA
TTTGAACTACATCATCCACTGGGGCCAGGGAACCCTGGTCACCGTCTCCTCA
HCVR (V_{H}) Amino Acid Sequence
(SEQ ID NO: 221)
QVTLRESGPALVKPSQTLTLTCTFSGFSLNTYGMFVSWIRQPPGKALEWLAHIHWDDDKYYSTSLKTRLTISKDTSK
NQVVLTMTNMDPVDTATYYCARGHNNLNYIIHWGQGTLVTVSS
or
(SEQ ID NO: 685)
QVQLVESGPALVKPSQTLTLTCTFSGFSLNTYGMFVSWIRQPPGKALEWLAHIHWDDDKYYSTSLKTRLTISKDTSK
NQVVLTMTNMDPVDTATYYCARGHNNLNYIIHWGQGTLVTVSS
HCDR1:
(SEQ ID NO: 222)
GFSLNTYGMF
HCDR2:
(SEQ ID NO: 223)
IHWDDDK
HCDR3:
(SEQ ID NO: 224)
ARGHNNLNYIIH
LCVR (V_{L}) Nucleotide Sequence
(SEQ ID NO: 225)
GCCATCCAGATGACCCAGTCTCCATCCTCCCTGTCTGCATCTGTAGGAGACAGAGTCACCATCACTTGCCGGGCAAG
TCAGGGCATTAGAAATGATTTAGGCTGGTATCAGCAGAAACCAGGGAAAGCCCCTAAGCTCCTGATCTATGCTGCAT
CCACTTTACAAAGTGGGGTCCCATCAAGGTTCAGCGGCAGTGGATCTGGCACAGATTTCACTCTCACCATCAGCAGC
CTGCAGCCTGAAGATTTTGCAACTTATTACTGTCTACAAGATTACAATTACCCATTCACTTTCGGCCCTGGGACCAA
AGTGGATATCAAA
LCVR (V_{L}) Amino Acid Sequence
(SEQ ID NO: 226)
AIQMTQSPSSLSASVGDRVTITCRASQGIRNDLGWYQQKPGKAPKLLIYAASTLQSGVPSRFSGSGSGTDFTLTISS
LQPEDFATYYCLQDYNYPFTFGPGTKVDIK
or
(SEQ ID NO: 686)
DILMTQSPSSLSASVGDRVTITCRASQGIRNDLGWYQQKPGKAPKLLIYAASTLQSGVPSRFSGSGSGTDFTLTISS
LQPEDFATYYCLQDYNYPFTFGPGTKVEIK
LCDR1:
(SEQ ID NO: 227)
QGIRND
LCDR2:
(SEQ ID NO: 228)
AAS
LCDR3:
(SEQ ID NO: 229)
LQDYNYPFT
69326
HCVR (V_{H}) Nucleotide Sequence
(SEQ ID NO: 230)
GAGGTGCAGCTGGTGGAGTCTGGGGGAGGCTTGGTACAGCCTGGAGGGTCCCTGAGACTCTCCTGTGCAGTCTCTGG
ATTCATCTTCAGTAGTTATGAAATGAACTGGGTCCGCCAGGCTCCAGGGAAGGGGCTGGAGTGGGTTTCATACATTA
GTAGTAGTGGTAGTACCATATTCTACGCAGACTCTGTGAAGGGCCGATTCACCATCTCCAGAGACAACGCCAAGAAC
TCACTGTATCTGCAAATGAACAGCCTGAGAGCCGAGGACACGGCTGTTTATTACTGTGTGTCTGGAGTGGTCCTTTT
TGATGTCTGGGGCCAAGGGACAATGGTCACCGTCTCTTCA
HCVR (V_{H}) Amino Acid Sequence
(SEQ ID NO: 231)
EVQLVESGGGLVQPGGSLRLSCAVSGFIFSSYEMNWVRQAPGKGLEWVSYISSSGSTIFYADSVKGRFTISRDNAKN
SLYLQMNSLRAEDTAVYYCVSGVVLFDVWGQGTMVTVSS
or
(SEQ ID NO: 687)
QVQLVESGGGLVQPGGSLRLSCAVSGFIFSSYEMNWVRQAPGKGLEWVSYISSSGSTIFYADSVKGRFTISRDNAKN
SLYLQMNSLRAEDTAVYYCVSGVVLFDVWGQGTMVTVSS
HCDR1:
(SEQ ID NO: 232)
GFIFSSYE
HCDR2:
(SEQ ID NO: 233)
ISSSGSTI
HCDR3:
(SEQ ID NO: 234)
VSGVVLFDV
LCVR (V_{L}) Nucleotide Sequence
(SEQ ID NO: 235)
GAAATAGTGATGACGCAGTCTCCAGCCACCCTGTCTGTGTCTCCGGGGGAAAGAGCCACCCTCTCCTGCAGGGCCAG
TCAGAGTGTTAGCAGCAACTTTGCCTGGTACCAACAGAAACCTGGCCAGGCTCCCAGGCTCCTCATCTATAGTGCAT
CCTCCAGGGCCACTGGTATCCCAGTCAGGTTCAGTGGCAGTGGGTCTGGGACAGAGTTCACTCTCACCATCAGCAGC
CTGCAGTCTGAAGATTTTGCAGTTTATTACTGTCAGCAGTATAATATCTGGCCTCGGACGTTCGGCCAAGGGACCAA
GGTGGAAATCAAA
LCVR (V_{L}) Amino Acid Sequence
(SEQ ID NO: 236)
EIVMTQSPATLSVSPGERATLSCRASQSVSSNFAWYQQKPGQAPRLLIYSASSRATGIPVRFSGSGSGTEFTLTISS
LQSEDFAVYYCQQYNIWPRTFGQGTKVEIK
or
(SEQ ID NO: 688)
DIVMTQSPATLSVSPGERATLSCRASQSVSSNFAWYQQKPGQAPRLLIYSASSRATGIPVRFSGSGSGTEFTLTISS
LQSEDFAVYYCQQYNIWPRTFGQGTKVEIK
LCDR1:
(SEQ ID NO: 237)
QSVSSN
LCDR2:
(SEQ ID NO: 238)
SAS
LCDR3:
(SEQ ID NO: 239)
QQYNIWPRT
69329
HCVR (V_{H}) Nucleotide Sequence
(SEQ ID NO: 240)
GAGGTGCAGCTGGTGGAGTCTGGGGGAGGCTTGGTCCAGCCTGGGGGGTCCCTGAGACTCTCCTGTGCAGCCTCTGG
ATTCACCTTTAGTAACTATTGGATGACCTGGGTCCGCCAGGCTCCAGGGAAGGGGCTGGAGTGGGTGGCCAACATAA
AGGAAGATGGAAGTGAGAAAGACTATGTGGACTCTGTGAAGGGCCGATTCACCATCTCCAGAGACAACGCCAAGAAC
TCACTGTATCTGCAAATGAACAGCCTGAGAGGCGAGGACACGGCTGTGTATTACTGTGCGAGAGATGGGGAGCAGCT
CGTCGATTACTACTACTACTACGTTATGGACGTCTGGGGCCAAGGGACCACGGTCACCGTCTCCTCA
HCVR (V_{H}) Amino Acid Sequence
(SEQ ID NO: 241)
EVQLVESGGGLVQPGGSLRLSCAASGFTFSNYWMTWVRQAPGKGLEWVANIKEDGSEKDYVDSVKGRFTISRDNAKN
SLYLQMNSLRGEDTAVYYCARDGEQLVDYYYYYVMDVWGQGTTVTVSS
or
(SEQ ID NO: 689)
QVQLVESGGGLVQPGGSLRLSCAASGFTFSNYWMTWVRQAPGKGLEWVANIKEDGSEKDYVDSVKGRFTISRDNAKN
SLYLQMNSLRGEDTAVYYCARDGEQLVDYYYYYVMDVWGQGTTVTVSS
HCDR1:
(SEQ ID NO: 242)
GFTFSNYW
HCDR2:
(SEQ ID NO: 243)
IKEDGSEK
HCDR3:
(SEQ ID NO: 244)
ARDGEQLVDYYYYYVMDV
LCVR (V_{L}) Nucleotide Sequence
(SEQ ID NO: 245)
GACATCCAGATGACCCAGTCTCCATCTTCCGTGTCTGCATCTGTAGGAGACAGAGTCACCATCACTTGTCGGGCGAG
TCAGGGTATTAGCAGCTGGTTAGCCTGGTATCAGCAGAAACCAGGGAAAGCCCCTAAGCTCCTGATCTATGCTGCAT
CCAGTTTGCAAAGTGGGGTCCCATCAAGGTTCAGCGGCAGTGGATCTGGGACAGATTTCACTCTCACCATCAGCAGC
CTGCAGCCTGAAGATTTTGCAACTTACTATTGTCAAAAGGCTAACAGTTTCCCGTACACTTTTGGCCAGGGGACCAA
GCTGGAGATCAAA
LCVR (V_{L}) Amino Acid Sequence
(SEQ ID NO: 246)
DIQMTQSPSSVSASVGDRVTITCRASQGISSWLAWYQQKPGKAPKLLIYAASSLQSGVPSRFSGSGSGTDFTLTISS
LQPEDFATYYCQKANSFPYTFGQGTKLEIK
or
(SEQ ID NO: 690)
DIQMTQSPSSVSASVGDRVTITCRASQGISSWLAWYQQKPGKAPKLLIYAASSLQSGVPSRFSGSGSGTDFTLTISS
LQPEDFATYYCQKANSFPYTFGQGTKVEIK
LCDR1:
(SEQ ID NO: 247)
QGISSW
LCDR2:
(SEQ ID NO: 248)
AAS
LCDR3:
(SEQ ID NO: 249)
QKANSFPYT
69323 (REGN16816 anti-hTfR scFv:hGAA)
HCVR (V_{H}) Nucleotide Sequence
(SEQ ID NO: 250)
GAAGTGCAGCTGGTGGAGTCTGGGGGAGGCTTGGTACAGCCTGGCAGGTCCCTGAGACTCTCCTGTGCAGCCTCTGG
ATTCACCTTTGATGACTATGCCATGCACTGGGTCCGGCAAGCTCCAGGGAAGGGCCTGGAGTGGGTCTCAGGTATTA
GTTGGAATAGTGGTTACATAGGCTATGCGGACTCTGTGAAGGGCCGATTCACCATCTCCAGAGACAACGCCGAGAAC
TCCCTACATCTGCAAATGAACAGTCTGAGAGCTGAGGACACGGCCTTGTATTACTGTGCAAGAGGGGGATCTACTCT
GGTTCGGGGAGTTAAGGGAGGCTACTACGGTATGGACGTCTGGGGCCAAGGGACCACGGTCACCGTCTCCTCA
HCVR (V_{H}) Amino Acid Sequence
(SEQ ID NO: 251)
EVQLVESGGGLVQPGRSLRLSCAASGFTFDDYAMHWVRQAPGKGLEWVSGISWNSGYIGYADSVKGRFTISRDNAEN
SLHLQMNSLRAEDTALYYCARGGSTLVRGVKGGYYGMDVWGQGTTVTVSS
HCDR1:
(SEQ ID NO: 252)
GFTFDDYA
HCDR2:
(SEQ ID NO: 253)
ISWNSGYI
HCDR3:
(SEQ ID NO: 254)
ARGGSTLVRGVKGGYYGMDV
LCVR (V_{L}) Nucleotide Sequence
(SEQ ID NO: 255)
GACATCCAGATGACCCAGTCTCCATCCTCCCTGTCTGCATCTGTAGGAGACAGAGTCACCATCACTTGCCGGGCAAG
TCAGAGCATAAGTAGCTATTTAAATTGGTATCAGCAGAAACCAGGTAAAGCCCCTAAGGTCCTGATCTATGCTGCAT
CCAGTTTGCAAAGTGGGGTCCCATCAAGGTTCAGTGGCAGTGGATCTGGGACAGATTTCACTCTCACCATCAGCAGT
CTGCAACCTGAAGATTTTGCAACTTACTACTGTCAACAGAGTTACAGTATTCCGCTCACTTTCGGCGGAGGGACCAA
GGTGGAGATCAAA
LCVR (V_{L}) Amino Acid Sequence
(SEQ ID NO: 256)
DIQMTQSPSSLSASVGDRVTITCRASQSISSYLNWYQQKPGKAPKVLIYAASSLQSGVPSRFSGSGSGTDFTLTISS
LQPEDFATYYCQQSYSIPLTFGGGTKVEIK
LCDR1:
(SEQ ID NO: 257)
QSISSY
LCDR2:
(SEQ ID NO: 258)
AAS
LCDR3:
(SEQ ID NO: 259)
QQSYSIPLT
69305
HCVR (V_{H}) Nucleotide Sequence
(SEQ ID NO: 260)
CAGGTGCAGCTGGTGGAGTCTGGGGGAGGCGTGGTCCAGCCTGGGAGGTCCCTGAGACTCTCCTGTGCAGCGTCTGG
ATTCACCTTCAGTAGCTATGGCATGCACTGGGTCCGCCAGGCTCCAGGCAAGGGGCTGGAGTGGGTGGCAGTTATAT
GGTATGATGGAAGTAATAAATACTATGCAGACTCCGTGAAGGGCCGATTCACCATCTCCAGAGACATTTCCAAGAAC
ACGCTGTATCTGCAAATGAACAGCCTGAGAGCCGAGGACACGGCCGTATATTACTGTGCGGGTCAACTGGATCTCTT
CTTTGACTACTGGGGCCAGGGAACCCTGGTCACCGTCTCCTCA
HCVR (V_{H}) Amino Acid Sequence
(SEQ ID NO: 261)
QVQLVESGGGVVQPGRSLRLSCAASGFTFSSYGMHWVRQAPGKGLEWVAVIWYDGSNKYYADSVKGRFTISRDISKN
TLYLQMNSLRAEDTAVYYCAGQLDLFFDYWGQGTLVTVSS
or
(SEQ ID NO: 691)
EVQLVESGGGVVQPGRSLRLSCAASGFTFSSYGMHWVRQAPGKGLEWVAVIWYDGSNKYYADSVKGRFTISRDISKN
TLYLQMNSLRAEDTAVYYCAGQLDLFFDYWGQGTLVTVSS
HCDR1:
(SEQ ID NO: 262)
GFTFSSYG
HCDR2:
(SEQ ID NO: 263)
IWYDGSNK
HCDR3:
(SEQ ID NO: 264)
AGQLDLFFDY
LCVR (V_{L}) Nucleotide Sequence
(SEQ ID NO: 265)
GACATCCAGATGACCCAGTCTCCATCCTCCCTGTCTGCATCTGTAGGAGACAGAGTCACCATCACTTGCCGGGCAAG
TCAGAGCATTGACAGGTATTTAAATTGGTATCGGCAGAAACCAGGGAAAGCCCCTAAGCTCCTGATCTATACTACAT
CCAGTTTGCAAAGTGGGGTCCCATCAAGGTTCAGTGGCAGTGGATCTGGGACAGATTTCACTCTCACCCTCAGCAGT
CTGCAGCCTGAAGATTTTGCAACTTACTACTGTCAGCAGAGTTACAGTCCCCCGCTCACTTTCGGCGGAGGGACCAA
GGTGGAGATCAAA
LCVR (V_{L}) Amino Acid Sequence
(SEQ ID NO: 266)
DIQMTQSPSSLSASVGDRVTITCRASQSIDRYLNWYRQKPGKAPKLLIYTTSSLQSGVPSRFSGSGSGTDFTLTLSS
LQPEDFATYYCQQSYSPPLTFGGGTKVEIK
LCDR1:
(SEQ ID NO: 267)
QSIDRY
LCDR2:
(SEQ ID NO: 268)
TTS
LCDR3:
(SEQ ID NO: 269)
QQSYSPPLT
69307 (REGN16817 anti-hTfR scFv:hGAA)
HCVR (V_{H}) Nucleotide Sequence
(SEQ ID NO: 270)
GAGGTGCAGCTGGTGGAGTCTGGGGGAGGCTTGGTCCAGCCTGGGGGGTCCCTGAGACTCTCCTGTACAGCCTCTGG
ATTCACCTTTAGTAACTATTGGATGACCTGGGTCCGCCAGGCTCCAGGGAAGGGGCTGGAGTGGGTGGCCAACATAA
AGGAAGATGGAAGTGAGAAAGAGTATGTGGACTCTGTGAAGGGCCGGTTCACCATCTCCAGAGACAACGCCAAGAAT
TCACTGTATCTGCAAATGAACAGCCTGAGAGGCGAGGACACGGCTGTATATTACTGTGCGAGAGATGGGGAGCAGCT
CGTCGATTACTATTACTACTACGTTATGGACGTCTGGGGCCAAGGGACCACGGTCACCGTCTCCTCA
HCVR (V_{H}) Amino Acid Sequence
(SEQ ID NO: 271)
EVQLVESGGGLVQPGGSLRLSCTASGFTFSNYWMTWVRQAPGKGLEWVANIKEDGSEKEYVDSVKGRFTISRDNAKN
SLYLQMNSLRGEDTAVYYCARDGEQLVDYYYYYVMDVWGQGTTVTVSS
HCDR1:
(SEQ ID NO: 272)
GFTFSNYW
HCDR2:
(SEQ ID NO: 273)
IKEDGSEK
HCDR3:
(SEQ ID NO: 274)
ARDGEQLVDYYYYYVMDV
LCVR (V_{L}) Nucleotide Sequence
(SEQ ID NO: 275)
GACATCCAGATGACCCAGTCTCCATCTTCCGTGTCTGCATCTGTTGGAGACAGAGTCACCATCACTTGTCGGGCGAG
TCAGGGTATTAGCAGCTGGTTAGCCTGGTATCAGCAGAAACCAGGGAAAGCCCCTAAGCTCCTGATCTATGCTGCAT
CCAGTTTGCAAAGTGGGGTCCCATCAAGGTTCAGCGGCAGTGGATCTGGGACAGATTTCACTCTCACCATCAGCAGC
CTGCAGCCTGAAGATTTTGCAACTTACTATTGTCAAAAGGCTGACAGTCTCCCGTACGCTTTTGGCCAGGGGACCAA
GCTGGAGATCAAA
LCVR (V_{L}) Amino Acid Sequence
(SEQ ID NO: 276)
DIQMTQSPSSVSASVGDRVTITCRASQGISSWLAWYQQKPGKAPKLLIYAASSLQSGVPSRFSGSGSGTDFTLTISS
LQPEDFATYYCQKADSLPYAFGQGTKLEIK
LCDR1:
(SEQ ID NO: 277)
QGISSW
LCDR2:
(SEQ ID NO: 278)
AAS
LCDR3:
(SEQ ID NO: 279)
QKADSLPYA
12795B
HCVR (V_{H}) Nucleotide Sequence
(SEQ ID NO: 280)
GAGGTGCAGCTGGTGGAGTCTGGGGGAGGCTTGGTTCAGCCTGGGGGGTCCCTGAGACTCTCCTGTGCAACCTCTGG
ATTCACCTTTACCAGCTATGACATGAAGTGGGTCCGCCAGGCTCCAGGGCTGGGCCTGGAGTGGGTCTCAGCTATTA
GTGGTAGTGGTGGTAACACATACTACGCAGACTCCGTGAAGGGCCGGTTCACCATCTCCAGAGACAATTCCAGGAAC
ACGCTGTATCTGCAAATGAACAGCCTGAGAGCCGAGGACACGGCCGTATATTACTGTACGAGGTCCCATGACTTCGG
TGCCTTCGACTACTTTGACTACTGGGGCCAGGGAACCCTGGTCACCGTCTCCTCA
HCVR (V_{H}) Amino Acid Sequence
(SEQ ID NO: 281)
EVQLVESGGGLVQPGGSLRLSCATSGFTFTSYDMKWVRQAPGLGLEWVSAISGSGGNTYYADSVKGRFTISRDNSRN
TLYLQMNSLRAEDTAVYYCTRSHDFGAFDYFDYWGQGTLVTVSS
or
(SEQ ID NO: 692)
EVQLVQSGGGLVQPGGSLRLSCATSGFTFTSYDMKWVRQAPGLGLEWVSAISGSGGNTYYADSVKGRFTISRDNSRN
TLYLQMNSLRAEDTAVYYCTRSHDFGAFDYFDYWGQGTMVTVSS
HCDR1:
(SEQ ID NO: 282)
GFTFTSYD
HCDR2:
(SEQ ID NO: 283)
ISGSGGNT
HCDR3:
(SEQ ID NO: 284)
TRSHDFGAFDYFDY
LCVR (V_{L}) Nucleotide Sequence
(SEQ ID NO: 285)
GACATCCAGATGACCCAGTCTCCATCCTCCCTGTCTGCATCTGTGGGAGACAGAGTCACCATCACTTGCCGGGCAAG
TCAGGGCATTAGAGATCATTTTGGCTGGTATCAGCAGAAACCAGGGAAAGCCCCTAAGCGCCTGATCTATGCTGCAT
CCAGTTTGCACAGTGGGGTCCCATCAAGGTTCAGCGGCAGTGGATCTGGGACAGAATTCACTCTCACAATCAGCAGC
TTGCAGCCTGAAGATTTTGCAACCTATTACTGTCTACAGTATGATACTTACCCGCTCACTTTCGGCGGAGGGACCAA
GGTGGAGATCAAA
LCVR (V_{L}) Amino Acid Sequence
(SEQ ID NO: 286)
DIQMTQSPSSLSASVGDRVTITCRASQGIRDHFGWYQQKPGKAPKRLIYAASSLHSGVPSRFSGSGSGTEFTLTISS
LQPEDFATYYCLQYDTYPLTFGGGTKVEIK
or
(SEQ ID NO: 693)
DIQLTQSPSSLSASVGDRVTITCRASQGIRDHFGWYQQKPGKAPKRLIYAASSLHSGVPSRFSGSGSGTEFTLTISS
LQPEDFATYYCLQYDTYPLTFGGGTKVEIK
LCDR1:
(SEQ ID NO: 287)
QGIRDH
LCDR2:
(SEQ ID NO: 288)
AAS
LCDR3:
(SEQ ID NO: 289)
LQYDTYPLT
12798B (REGN17078 Fab; REGN17072 scFv; REGN16818 anti-hTfR scFv:hGAA)
HCVR (V_{H}) Nucleotide Sequence
(SEQ ID NO: 290)
GAAGTGCAGCTGGTGGAGTCTGGGGGAGACTTGGTACAGCCTGGCAGGTCCCTGAGACTCTCCTGTGCAGCCTCTGG
ATTCACCTTTGATGATTATGCCATGCACTGGGTCCGGCAAGCTCCAGGGAAGGGCCTGGAGTGGGTCTCAGGTATTA
GTTGGAATAGTGCTACCAGAGTCTATGCGGACTCTGTGAAGGGCCGATTCACCATCTCCAGAGACAACGCCAAGAAT
TTCCTGTATCTGCAAATGAACAGTCTGAGATCTGAGGACACGGCCTTGTATCACTGTGCAAAAGATATGGATATCTC
GCTAGGGTACTACGGTTTGGACGTCTGGGGCCAAGGGACCACGGTCACCGTCTCCTCA
HCVR (V_{H}) Amino Acid Sequence
(SEQ ID NO: 291)
EVQLVESGGDLVQPGRSLRLSCAASGFTFDDYAMHWVRQAPGKGLEWVSGISWNSATRVYADSVKGRFTISRDNAKN
FLYLQMNSLRSEDTALYHCAKDMDISLGYYGLDVWGQGTTVTVSS
HCDR1:
(SEQ ID NO: 292)
GFTFDDYA
HCDR2:
(SEQ ID NO: 293)
ISWNSATR
HCDR3:
(SEQ ID NO: 294)
AKDMDISLGYYGLDV
LCVR (V_{L}) Nucleotide Sequence
(SEQ ID NO: 295)
GAAATAGTGATGACGCAGTCTCCAGCCACCCTGTCTGTGTCTCCAGGGGAAAGAGCCACCCTCTCCTGCAGGGCCAG
TCAGACTGTTAGCAGCAACTTAGCCTGGTATCAGCAGAAACCTGGCCAGGCTCCCAGGCTCCTCATCTATGGTTCAT
CCTCCAGGGCCACTGGTATCCCAGCCAGGTTCAGTGGCAGTGGGTCTGGGACAGAGTTCACTCTCACCATCAGCAGC
CTGCAGTCTGAAGATTTTGCAGTTTATTACTGTCAGCAGTATAATAACTGGCCTCCCTACACTTTTGGCCAGGGGAC
CAAGCTGGAGATCAAA
LCVR (V_{L}) Amino Acid Sequence
(SEQ ID NO: 296)
EIVMTQSPATLSVSPGERATLSCRASQTVSSNLAWYQQKPGQAPRLLIYGSSSRATGIPARFSGSGSGTEFTLTISS
LQSEDFAVYYCQQYNNWPPYTFGQGTKLEIK
LCDR1:
(SEQ ID NO: 297)
QTVSSN
LCDR2:
(SEQ ID NO: 298)
GSS
LCDR3:
(SEQ ID NO: 299)
QQYNNWPPYT
12799B (REGN17079 Fab; REGN17073 scFv; REGN16819 anti-hTfR scFv:hGAA)
HCVR (V_{H}) Nucleotide Sequence
(SEQ ID NO: 300)
CAGATCACCTTGAAGGAGTCTGGTCCTACGCTGGTGAAACCCACACAGACCCTCACGCTGACCTGCACCTTCTCTGG
GTTCTCACTCAGCACTAGTGGAGTGGGTGTGGTCTGGATCCGTCAGCCCCCCGGAAAGGCCCTGGAGTGGCTTGCAC
TCATTTATTGGAATGATCATAAGCGGTACAGCCCATCTCTGGGGAGCAGGCTCACCATCACCAAGGACACCTCCAAA
AACCAGGTGGTCCTTACAATGACCAACATGGACCCTGTGGACACAGCCACATATTACTGTGCACACTACAGTGGGAG
CTATTCCTACTACTACTATGGTTTGGACGTCTGGGGCCAAGGGACCACGGTCACCGTCTCCTCA
HCVR (V_{H}) Amino Acid Sequence
(SEQ ID NO: 301)
QITLKESGPTLVKPTQTLTLTCTFSGFSLSTSGVGVVWIRQPPGKALEWLALIYWNDHKRYSPSLGSRLTITKDTSK
NQVVLTMTNMDPVDTATYYCAHYSGSYSYYYYGLDVWGQGTTVTVSS
HCDR1:
(SEQ ID NO: 302)
GFSLSTSGVG
HCDR2:
(SEQ ID NO: 303)
IYWNDHK
HCDR3:
(SEQ ID NO: 304)
AHYSGSYSYYYYGLDV
LCVR (V_{L}) Nucleotide Sequence
(SEQ ID NO: 305)
GACATCCAGATGACCCAGTCTCCATCTTCCGTGTCTGCATCTGTAGGAGACAGAGTCACCATCACTTGTCGGGCGAG
TCAGGGTATTGCCAGCTGGTTAGCCTGGTATCAGCAGAAACCAGGGAAAGCCCCTGAGCTCCTGATCTATGCTGCAT
CCAGTTTGCAAGGTGGGGTCCCATCAAGGTTCAGCGGCAGTGGATCTGGGACAGATTTCACTCTCACCATCAGCAGC
CTGCAGCCTGAAGATTTTGCAATTTACTATTGTCAACAGGCTAACTATTTCCCGTGGACGTTCGGCCAAGGGACCAA
GGTGGAAATCAAA
LCVR (V_{L}) Amino Acid Sequence
(SEQ ID NO: 306)
DIQMTQSPSSVSASVGDRVTITCRASQGIASWLAWYQQKPGKAPELLIYAASSLQGGVPSRFSGSGSGTDFTLTISS
LQPEDFAIYYCQQANYFPWTFGQGTKVEIK
LCDR1:
(SEQ ID NO: 307)
QGIASW
LCDR2:
(SEQ ID NO: 308)
AAS
LCDR3:
(SEQ ID NO: 309)
QQANYFPWT
12801B
HCVR (V_{H}) Nucleotide Sequence
(SEQ ID NO: 310)
GAGGTGCAGCTGTTGGAGTCTGGGGGAGCCTTGGTACAGCCTGGGGGGTCCCTGAGACTCTCCTGTGCAGCCTCTGG
ATTCACCTTTACCTCCTATGCCATGCACTGGGTCCGCCAGGCTCCAGGGAAGGGTCTGGAGTGGGTCTCATCTATTA
GAGGTAGTGGTGGTGGCACATACTCCGCAGACTCCGTGAAGGGCCGGTTCACCATCTCCAGAGACAATTCCAGGGAC
ACTCTATATCTGCAAATGAACAGTGTGAGAGCCGAGGACACGGCCGTTTATTACTGTGCGAGGTCCCATGACTACGG
TGCCTTCGACTTCTTTGACTACTGGGGCCAGGGAACCCTGGTCACCGTCTCCTCA
HCVR (V_{H}) Amino Acid Sequence
(SEQ ID NO: 311)
EVQLLESGGALVQPGGSLRLSCAASGFTFTSYAMHWVRQAPGKGLEWVSSIRGSGGGTYSADSVKGRFTISRDNSRD
TLYLQMNSVRAEDTAVYYCARSHDYGAFDFFDYWGQGTLVTVSS
or
(SEQ ID NO: 694)
EVQLLESGGALVQPGGSLRLSCAASGFTFTSYAMHWVRQAPGKGLEWVSSIRGSGGGTYSADSVKGRFTISRDNSRD
TLYLQMNSVRAEDTAVYYCARSHDYGAFDFFDYWGQGTTVTVSS
HCDR1:
(SEQ ID NO: 312)
GFTFTSYA
HCDR2:
(SEQ ID NO: 313)
IRGSGGGT
HCDR3:
(SEQ ID NO: 314)
ARSHDYGAFDFFDY
LCVR (V_{L}) Nucleotide Sequence
(SEQ ID NO: 315)
GACATCCAGATGACCCAGTCTCCATCCTCCCTGTCTGCATCTGTAGGAGACAGAGTCACCATCACTTGCCGGGCAAG
TCAGGGCATTAGAACTGATTTAGGCTGGTATCAGCAGAAACCAGGGAAAGCCCCTAAGCGCCTGATCTATGCTGCAT
CCAGTTTGCAAAGTGGGGTCCCATCAAGGTTCAGCGGCAGTGGATCTGGGACAGAATTCACTCTCACAATCAGCAGC
CTGCGGCCTGAAGATTTTGCAACTTTTTACTGTCTACAGTATAATAGTTACCCGCTCACTTTCGGCGGAGGGACCAA
GGTGGAGATCAAA
LCVR (V_{L}) Amino Acid Sequence
(SEQ ID NO: 316)
DIQMTQSPSSLSASVGDRVTITCRASQGIRTDLGWYQQKPGKAPKRLIYAASSLQSGVPSRFSGSGSGTEFTLTISS
LRPEDFATFYCLQYNSYPLTFGGGTKVEIK
or
(SEQ ID NO: 695)
DIQMTQSPSSLSASVGDRVTITCRASQGIRTDLGWYQQKPGKAPKRLIYAASSLQSGVPSRFSGSGSGTEFTLTISS
LRPEDFATFYCLQYNSYPLTFGGGTKVDIK
LCDR1:
(SEQ ID NO: 317)
QGIRTD
LCDR2:
(SEQ ID NO: 318)
AAS
LCDR3:
(SEQ ID NO: 319)
LQYNSYPLT
12802B (REGN16820 anti-hTfR scFv:hGAA)
HCVR (V_{H}) Nucleotide Sequence
(SEQ ID NO: 320)
CAGGTGCAGCTGGTGGAGTCTGGGGGAGGCTTGGTCAAGCCTGGAGGGTCCCTGAGACTCTCCTGTGCAGCCTCTGG
ATTCACCTTCAGTGACTACTTCATGAGCTGGATCCGCCAGGCTCCAGGGAAGGGGCTGGAGTGGGTTTCATACATTA
GTAGTACTGGTAGTACCATAAATTATGCAGACTCTGTGAAGGGCCGATTCACCATCTCCAGGGACAATGTCAAGAAT
TCACTGTATCTGCAAATGACCAGCCTGAGAGTCGAGGACACGGCCGTGTATTACTGTACGAGAGATAACTGGAACTA
TGAATACTGGGGCCAGGGAACCCTGGTCACCGTCTCCTCA
HCVR (V_{H}) Amino Acid Sequence
(SEQ ID NO: 321)
QVQLVESGGGLVKPGGSLRLSCAASGFTFSDYFMSWIRQAPGKGLEWVSYISSTGSTINYADSVKGRFTISRDNVKN
SLYLQMTSLRVEDTAVYYCTRDNWNYEYWGQGTLVTVSS
HCDR1:
(SEQ ID NO: 322)
GFTFSDYF
HCDR2:
(SEQ ID NO: 323)
ISSTGSTI
HCDR3:
(SEQ ID NO: 324)
TRDNWNYEY
LCVR (V_{L}) Nucleotide Sequence
(SEQ ID NO: 325)
GAAATAGTGATGACGCAGTCTCCAGCCACCCTGTCTGTGTCTCCAGGGGAAAGAGCCACCCTCTCCTGCAGGGCCAG
TCAGAGTGTTAGCATCAACTTAGCCTGGTACCAGCAGAAACCTGGCCAGGCTCCCAGGCTCCTCATCTTTGTTGCAT
CCACCAGGGCCACTGGTATCCCAGCCAGGTTCAGTGGCAGTGGGTCTGGGACAGAGTTCACTCTCACCATCAGCAGC
CTGCAGTCTGAAGATTTTGCAACTTATTACTGTCAGCAGTATGATATCTGGCCGTACACTTTTGGCCAGGGGACCAA
GCTGGAGATCAAA
LCVR (V_{L}) Amino Acid Sequence
(SEQ ID NO: 326)
EIVMTQSPATLSVSPGERATLSCRASQSVSINLAWYQQKPGQAPRLLIFVASTRATGIPARFSGSGSGTEFTLTISS
LQSEDFATYYCQQYDIWPYTFGQGTKLEIK
LCDR1:
(SEQ ID NO: 327)
QSVSIN
LCDR2:
(SEQ ID NO: 328)
VAS
LCDR3:
(SEQ ID NO: 329)
QQYDIWPYT
12808B
HCVR (V_{H}) Nucleotide Sequence
(SEQ ID NO: 330)
CAGCTGCAGCTGCAGGAGTCGGGCCCAGGACTGGTGAAGCCTTCGGAGACCCTGTCCCTCACCTGCACTGTGTCTGG
TGAATCCATCAGCAGTAATACTTACTACTGGGGCTGGATCCGCCAGCCCCCAGGGAAGGGGCTGGAATGGATTGGGA
GTATCGATTATAGTGGGACCACCAATTATAACCCGTCCCTCAAGAGTCGAGTCACCATATCCGTAGACACGTCCAGG
AATCACTTCTCCCTGAGGCTGAGGTCTGTGACCGCCGCAGACACGGCTGTGTATTACTGTGCGAGAGAGTGGGGAAA
CTACGGCTACTATTACGGTATGGACGTTTGGGGCCAAGGGACCACGGTCACCGTCTCCTCA
HCVR (V_{H}) Amino Acid Sequence
(SEQ ID NO: 331)
QLQLQESGPGLVKPSETLSLTCTVSGESISSNTYYWGWIRQPPGKGLEWIGSIDYSGTTNYNPSLKSRVTISVDTSR
NHFSLRLRSVTAADTAVYYCAREWGNYGYYYGMDVWGQGTTVTVSS
or
(SEQ ID NO: 696)
QVQLVESGPGLVKPSETLSLTCTVSGESISSNTYYWGWIRQPPGKGLEWIGSIDYSGTTNYNPSLKSRVTISVDTSR
NHFSLRLRSVTAADTAVYYCAREWGNYGYYYGMDVWGQGTTVTVSS
HCDR1:
(SEQ ID NO: 332)
GESISSNTYY
HCDR2:
(SEQ ID NO: 333)
IDYSGTT
HCDR3:
(SEQ ID NO: 334)
AREWGNYGYYYGMDV
LCVR (V_{L}) Nucleotide Sequence
(SEQ ID NO: 335)
GACATCCAGATGACCCAGTCTCCATCCTCCCTGTCTGCATCTGTAGGAGACAGAGTCACCATCAATTGCCGGGCAAG
TCAGGGCATTAGAAATGATTTAGGCTGGTATCAGCAGAAACCAGGGAAAGCCCCTAAGCGCCTGATCTATGCTGCAT
CCAGTTTGCAAAGTGGGGTCCCATTAAGGTTCAGTGGCAGTGGATCTGGGACAGAATTCACTCTCACAATCAACAAC
CTGCAGCCTGAAGATTTTGCAACTTATTACTGTCTATCGCATAATAGTTACCCGTGGACGTTCGGCCAAGGGACCAA
GGTGGAAATCAAA
LCVR (V_{L}) Amino Acid Sequence
(SEQ ID NO: 336)
DIQMTQSPSSLSASVGDRVTINCRASQGIRNDLGWYQQKPGKAPKRLIYAASSLQSGVPLRFSGSGSGTEFTLTINN
LQPEDFATYYCLSHNSYPWTFGQGTKVEIK
LCDR1:
(SEQ ID NO: 337)
QGIRND
LCDR2:
(SEQ ID NO: 338)
AAS
LCDR3:
(SEQ ID NO: 339)
LSHNSYPWT
12812B (REGN16821 anti-hTfR scFv:hGAA)
HCVR (V_{H}) Nucleotide Sequence
(SEQ ID NO: 340)
CAGGTGCAGCTGGTGCAGTCTGGGGCTGAGGTGAAGAAGCCTGGGTCCTCGGTGAGGGTCTCCTGCAAGGCTTCTAG
AGGCACCTTCAGCAGCTATGCTATCAGCTGGGTGCGACAGGCCCCTGGACAAGGCCTTGAGTGGATGGGAGGGATCA
TCCCCATCTTTGGTACAGCAAACTACGCACAGAAGTTCCTGGCCAGAGTCACGATTACCGCGGACGAATCCACGAGC
ACAGCCTACATGGAGCTGAGCAGCCTGAGATCTGAGGACACGGCCGTGTATTACTGTGCGAGAGAGAAGGGGTGGAA
CTACTTTGACTACTGGGGCCAGGGAACCCTGGTCACCGTCTCCTCA
HCVR (V_{H}) Amino Acid Sequence
(SEQ ID NO: 341)
QVQLVQSGAEVKKPGSSVRVSCKASRGTESSYAISWVRQAPGQGLEWMGGIIPIFGTANYAQKFLARVTITADESTS
TAYMELSSLRSEDTAVYYCAREKGWNYFDYWGQGTLVTVSS
HCDR1:
(SEQ ID NO: 342)
RGTFSSYA
HCDR2:
(SEQ ID NO: 343)
IIPIFGTA
HCDR3:
(SEQ ID NO: 344)
AREKGWNYFDY
LCVR (V_{L}) Nucleotide Sequence
(SEQ ID NO: 345)
GACATCCAGATGACCCAGTCTCCACCTTCCGTGTCTGCATCTGTAGGAGACAGAGTCACCATCACTTGTCGGGCGAG
TCAGGGTATTAGCAGCTGGTTAGCCTGGTATCAGCAGAAACCAGGGAAAGCCCCTAAACTCCTGATCTATGCTGCAT
CCAGTTTGCAAAGTGGGGTCCCATCAAGGTTCAGCGGCAGTGGATCTGGGACAGATTTCACTCTCACCATCAGCAGC
CTGCAGCCTGAAGATTTTGCAACTTACTATTGTCAACAGGCTAACAGTTTCCCTCGGACGTTCGGCCAAGGGACCAA
GGTGGAAATCAAA
LCVR (V_{L}) Amino Acid Sequence
(SEQ ID NO: 346)
DIQMTQSPPSVSASVGDRVTITCRASQGISSWLAWYQQKPGKAPKLLIYAASSLQSGVPSRFSGSGSGTDFTLTISS
LQPEDFATYYCQQANSFPRTFGQGTKVEIK
LCDR1:
(SEQ ID NO: 347)
QGISSW
LCDR2:
(SEQ ID NO: 348)
AAS
LCDR3:
(SEQ ID NO: 349)
QQANSFPRT
12816B
HCVR (V_{H}) Nucleotide Sequence
(SEQ ID NO: 350)
CAGGTGCAGCTGGTGGAGTCTGGGGGAGGCTTGGTCAAGCCTGGAGGGTCCCTGAGACTCTCCTGTGCAGCCTCTGG
ATTCACCTTCAGTGACTACTACATGAACTGGATCCGCCAGGCTCCAGGGAAGGGGCTGGAGTGGGTTTCATACATTA
GTAGTAGTGGGACTACCATATACTACGCAGACTCTGTGAAGGGCCGATTCACCATCTCCAGGGACAACGCCAAGAAA
TCACTGTATCTGGAGATGAACAGCCTCAGAGCCGAGGACACGGCCGTGTACTACTGTGCGAGAGAGGGGTACGGTAA
TGACTACTATTACTACGGTATAGACGTCTGGGGCCAAGGGACCACGGTCACCGTCTCCTCA
HCVR (V_{H}) Amino Acid Sequence
(SEQ ID NO: 351)
QVQLVESGGGLVKPGGSLRLSCAASGFTFSDYYMNWIRQAPGKGLEWVSYISSSGTTIYYADSVKGRFTISRDNAKK
SLYLEMNSLRAEDTAVYYCAREGYGNDYYYYGIDVWGQGTTVTVSS
or
(SEQ ID NO: 697)
EVQLVESGGGLVKPGGSLRLSCAASGFTFSDYYMNWIRQAPGKGLEWVSYISSSGTTIYYADSVKGRFTISRDNAKK
SLYLEMNSLRAEDTAVYYCAREGYGNDYYYYGIDVWGQGTTVTVSS
HCDR1:
(SEQ ID NO: 352)
GFTFSDYY
HCDR2:
(SEQ ID NO: 353)
ISSSGTTI
HCDR3:
(SEQ ID NO: 354)
AREGYGNDYYYYGIDV
LCVR (V_{L}) Nucleotide Sequence
(SEQ ID NO: 355)
GATATTGTGATGACTCAGTCTCCACTCTCCCTGCCCGTCACCCCTGGAGAGCCGGCCTCCATCTCCTGCAGGTCTAG
TCAGAGCCTCCTGCATGGTAATGGATACAACTATTTGACTTGGTACCTGCAGAAGCCAGGGCAGTCTCCACAGCTCC
TGATCTATTTGGGTTCTAATCGGGCCTCCGGGGTCCCTGACAGGTTCAGTGGCAGTGGATCAGGCACAGATTTTACA
CTGAAAATAAGCAGAGTGGAGGCTGAGGATGTTGGGGTTTATTACTGCATGCAAGCTCTACAAACTCCGTACACTTT
TGGCCAGGGGACCAAGCTGGAGATCAAA
LCVR (V_{L}) Amino Acid Sequence
(SEQ ID NO: 356)
DIVMTQSPLSLPVTPGEPASISCRSSQSLLHGNGYNYLTWYLQKPGQSPQLLIYLGSNRASGVPDRFSGSGSGTDFT
LKISRVEAEDVGVYYCMQALQTPYTFGQGTKLEIK
or
(SEQ ID NO: 698)
DIQLTQSPLSLPVTPGEPASISCRSSQSLLHGNGYNYLTWYLQKPGQSPQLLIYLGSNRASGVPDRFSGSGSGTDFT
LKISRVEAEDVGVYYCMQALQTPYTFGQGTKVEIK
LCDR1:
(SEQ ID NO: 357)
QSLLHGNGYNY
LCDR2:
(SEQ ID NO: 358)
LGS
LCDR3:
(SEQ ID NO: 359)
MQALQTPYT
12833B
HCVR (V_{H}) Nucleotide Sequence
(SEQ ID NO: 360)
CAGGTGCAGCTGGTGGAGTCTGGGGGAGGCGTGGTCCAGCCTGGGAGGTCCCTGAGACTCTCCTGTGCAGCCTCTGG
ATTCACCTTCAGTAGCTTTGGCATGCACTGGGTCCGCCAGGCTCCAGGCAAGGGGCTGGAGTGGGTGATATTTATAT
CATATGATGGAAGTGATAAATACTATGCAGACTCCGTGAAGGGCCGATTCGCCATCTCCAGAGACAGTTCCAAGAAC
ACGCTATATCTGCAAATGAACAGCCTGAGAGCTGAGGACACGGCTGTGTATTACTGTGCGAAAGAAAACGGTATTTT
GACTGATTCCTACGGTATGGACGTCTGGGGCCAAGGGACCACGGTCACCGTCTCCTCA
HCVR (V_{H}) Amino Acid Sequence
(SEQ ID NO: 361)
QVQLVESGGGVVQPGRSLRLSCAASGFTFSSFGMHWVRQAPGKGLEWVIFISYDGSDKYYADSVKGRFAISRDSSKN
TLYLQMNSLRAEDTAVYYCAKENGILTDSYGMDVWGQGTTVTVSS
or
(SEQ ID NO: 699)
EVQLVESGGGVVQPGRSLRLSCAASGFTFSSFGMHWVRQAPGKGLEWVIFISYDGSDKYYADSVKGRFAISRDSSKN
TLYLQMNSLRAEDTAVYYCAKENGILTDSYGMDVWGQGTTVTVSS
HCDR1:
(SEQ ID NO: 362)
GFTFSSFG
HCDR2:
(SEQ ID NO: 363)
ISYDGSDK
HCDR3:
(SEQ ID NO: 364)
AKENGILTDSYGMDV
LCVR (V_{L}) Nucleotide Sequence
(SEQ ID NO: 365)
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCAAAGTGGGGTCCCGTCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
LCVR (V_{L}) Amino Acid Sequence
(SEQ ID NO: 366)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIK
LCDR1:
(SEQ ID NO: 367)
QSISSY
LCDR2:
(SEQ ID NO: 368)
AAS
LCDR3:
(SEQ ID NO: 369)
QQSYSTPPIT
12834B
HCVR (V_{H}) Nucleotide Sequence
(SEQ ID NO: 370)
CAGGTTCAGCTGGTGCAGTCTGGAGCTGAGGTGAAGAAGCCTGGGGCCTCTGTGAAGGTCTCCTGCAAGGCTTCTGG
TTACACCTTTACCAGCTATGGTATCAGCTGGGTGCGACAGGCCCCTGGACAAGGGCTTGAGTGGATGGGATGGATCA
GTGTTTACCATGGTAACACAAACTATGCACAGAAGTTCCAGGGCAGAGTCACCATGACCACAGACACATCCACGAGC
ACAGCCTACATGGAGCTGAGGAGCCTGAGATCTGACGACACGGCCGTGTATTACTGTGCGAGAGAGGGGTATTACGA
TTTTTGGAGTGGTTATTACCCTTTTGACTACTGGGGCCAGGGAACCCTGGTCACCGTCTCCTCA
HCVR (V_{H}) Amino Acid Sequence
(SEQ ID NO: 371)
QVQLVQSGAEVKKPGASVKVSCKASGYTFTSYGISWVRQAPGQGLEWMGWISVYHGNTNYAQKFQGRVTMTTDTSTS
TAYMELRSLRSDDTAVYYCAREGYYDFWSGYYPFDYWGQGTLVTVSS
or
(SEQ ID NO: 700)
EVQLVESGAEVKKPGASVKVSCKASGYTFTSYGISWVRQAPGQGLEWMGWISVYHGNTNYAQKFQGRVTMTTDTSTS
TAYMELRSLRSDDTAVYYCAREGYYDFWSGYYPFDYWGQGTTVTVSS
HCDR1:
(SEQ ID NO: 372)
GYTFTSYG
HCDR2:
(SEQ ID NO: 373)
ISVYHGNT
HCDR3:
(SEQ ID NO: 374)
AREGYYDFWSGYYPFDY
LCVR (V_{L}) Nucleotide Sequence
(SEQ ID NO: 375)
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCAAAGTGGGGTCCCGTCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
LCVR (V_{L}) Amino Acid Sequence
(SEQ ID NO: 376)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIK
LCDR1:
(SEQ ID NO: 377)
QSISSY
LCDR2:
(SEQ ID NO: 378)
AAS
LCDR3:
(SEQ ID NO: 379)
QQSYSTPPIT
12835B
HCVR (V_{H}) Nucleotide Sequence
(SEQ ID NO: 380)
GAGGTGCAGCTGGTGGAGTCTGGGGGAGGCTTGATACAACCTGGAGGGTCCCTGAGACTCTCCTGTGAAGCCTCTGG
ATTCACCTTCAGAAATTATGAAATGAATTGGGTCCGCCAGGCTCCAGGGAAGGGGCTGGAGTGGGTTTCATATATTA
GTAGTAGTGGTAATATGAAAGACTACGCAGAGTCTGTGAAGGGCCGATTCACCATCTCCAGAGACAATGTCAAGAAT
TCACTGCAGCTGCAAATGAACAGCCTGAGAGTCGAGGACACGGCTGTTTATTACTGTGCGAGAGACGAGTTTCCTTA
CGGAATGGACGTCTGGGGCCAAGGGACCACGGTCACCGTCTCCTCA
HCVR (V_{H}) Amino Acid Sequence
(SEQ ID NO: 381)
EVQLVESGGGLIQPGGSLRLSCEASGFTFRNYEMNWVRQAPGKGLEWVSYISSSGNMKDYAESVKGRFTISRDNVKN
SLQLQMNSLRVEDTAVYYCARDEFPYGMDVWGQGTTVTVSS
HCDR1:
(SEQ ID NO: 382)
GFTFRNYE
HCDR2:
(SEQ ID NO: 383)
ISSSGNMK
HCDR3:
(SEQ ID NO: 384)
ARDEFPYGMDV
LCVR (V_{L}) Nucleotide Sequence
(SEQ ID NO: 385)
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCAAAGTGGGGTCCCGTCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
LCVR (V_{L}) Amino Acid Sequence
(SEQ ID NO: 386)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIK
LCDR1:
(SEQ ID NO: 387)
QSISSY
LCDR2:
(SEQ ID NO: 388)
AAS
LCDR3:
(SEQ ID NO: 389)
QQSYSTPPIT
12847B (REGN17083 anti-hTfR Fab; REGN17077 anti-hTfR scFv; REGN16826
anti-hTfR scFv:hGAA)
HCVR (V_{H}) Nucleotide Sequence
(SEQ ID NO: 390)
GAAGTGCAGCTGGTGGAGTCTGGGGGAGGCTTGGTTCAGCCTGGCAGGTCCCTGAGACTCTCCTGTGCAGCCTCTGG
ATTCACCTTTGATGATTATGCCATGAACTGGGTCCGGCAAGCTCCAGGGAAGGGCCTGGAGTGGGTCTCAGGTATTA
GTTGGAGTAGTGGTAGCATGGACTATGCGGACTCTGTGAAGGGCCGATTCACCATCTCCAGAGACAACGCCAAAAAC
TCCCTGTATCTGCAAATGAACAGTCTGAGAACTGAGGACACGGCCTTATATTACTGTGCAAAAGCTAGGGAAGTTGG
AGACTACTACGGTATGGACGTCTGGGGCCAAGGGACCACGGTCACCGTCTCCTCA
HCVR (V_{H}) Amino Acid Sequence
(SEQ ID NO: 391)
EVQLVESGGGLVQPGRSLRLSCAASGFTEDDYAMNWVRQAPGKGLEWVSGISWSSGSMDYADSVKGRFTISRDNAKN
SLYLQMNSLRTEDTALYYCAKAREVGDYYGMDVWGQGTTVTVSS
HCDR1:
(SEQ ID NO: 392)
GFTFDDYA
HCDR2:
(SEQ ID NO: 393)
ISWSSGSM
HCDR3:
(SEQ ID NO: 394)
AKAREVGDYYGMDV
LCVR (V_{L}) Nucleotide Sequence
(SEQ ID NO: 395)
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCAAAGTGGGGTCCCGTCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
LCVR (V_{L}) Amino Acid Sequence
(SEQ ID NO: 396)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIK
LCDR1:
(SEQ ID NO: 397)
QSISSY
LCDR2:
(SEQ ID NO: 398)
AAS
LCDR3:
(SEQ ID NO: 399)
QQSYSTPPIT
12848B (REGN16827 anti-hTfR scFv:hGAA)
HCVR (V_{H}) Nucleotide Sequence
(SEQ ID NO: 400)
GAAGTGCAGCTGGTGGAGTCTGGGGGAGGCTTGGTACAGCCTGGCAGGTCCCTGACACTCTCCTGTGCAGCCTCTGG
ATTCACCTTTGATAATTTTGGCATGCACTGGGTCCGGCAAGGTCCAGGGAAGGGCCTGGAATGGGTCTCAGGTCTTA
CTTGGAATAGTGGTGTCATAGGCTATGCGGACTCTGTGAAGGGCCGATTCACCATCTCCAGAGACAACGCCAAGAAC
TCCCTGTATCTGCAAATGAACAGTCTGAGACCTGAGGACACGGCCTTATATTACTGTGCAAAAGATATACGGAATTA
CGGCCCCTTTGACTACTGGGGCCAGGGAACCCTGGTCACCGTCTCCTCA
HCVR (V_{H}) Amino Acid Sequence
(SEQ ID NO: 401)
EVQLVESGGGLVQPGRSLTLSCAASGFTFDNFGMHWVRQGPGKGLEWVSGLTWNSGVIGYADSVKGRFTISRDNAKN
SLYLQMNSLRPEDTALYYCAKDIRNYGPFDYWGQGTLVTVSS
HCDR1:
(SEQ ID NO: 402)
GFTFDNFG
HCDR2:
(SEQ ID NO: 403)
LTWNSGVI
HCDR3:
(SEQ ID NO: 404)
AKDIRNYGPFDY
LCVR (V_{L}) Nucleotide Sequence
(SEQ ID NO: 405)
GAAATTGTGTTGACGCAGTCTCCAGGCACCCTGTCTTTGTCTCCAGGGGAAAGAGCCACCCTCTCCTGCAGGGCCAG
TCAGAGTGTTAGCAGCAGCTACTTAGCCTGGTACCAGCAGAAACCTGGCCAGGCTCCCAGGCTCCTCATCTATGGTG
CATCCAGCAGGGCCACTGGCATCCCAGACAGGTTCAGTGGCAGTGGGTCTGGGACAGACTTCACTCTCACCATCAGC
AGACTGGAGCCTGAAGATTTTGCAGTGTATTACTGTCAGCAGTATGGTAGCTCACCTTGGACGTTCGGCCAAGGGAC
CAAGGTGGAAATCAAA
LCVR (V_{L}) Amino Acid Sequence
(SEQ ID NO: 406)
EIVLTQSPGTLSLSPGERATLSCRASQSVSSSYLAWYQQKPGQAPRLLIYGASSRATGIPDRFSGSGSGTDFTLTIS
RLEPEDFAVYYCQQYGSSPWTFGQGTKVEIK
LCDR1:
(SEQ ID NO: 407)
QSVSSSY
LCDR2:
(SEQ ID NO: 408)
GAS
LCDR3:
(SEQ ID NO: 409)
QQYGSSPWT
12843B (REGN17075 anti-hTfR scFv; REGN16824 anti-hTfR scFv:hGAA;
REGN17081 anti-hTfR Fab)
HCVR (V_{H}) Nucleotide Sequence
(SEQ ID NO: 410)
GAGGTGCAGCTGGTGGAGTCTGGGGGAGGCTTAGTACAGCCTGGAGGGTCCCTAAGACTCTCCTGTGCAGCCTCTGG
ATTCACCTTCAATATTTTTGAAATGAACTGGGTCCGCCAGGCTCCAGGGAAGGGGCTGGAGTGGATTTCCTACATTA
GTAGTCGTGGAACTACCACATACTACGCAGACTCTGTGAGGGGCCGATTCACCATCTCCAGAGACAACGCCAAGAAC
TCACTGTATCTGCAAATGAACAGCCTGAGAGCCGAGGACACGGCTGTTTATTACTGTGCGAGAGATTATGAAGCAAC
AATCCCTTTTGACTTCTGGGGCCAGGGAACCCTGGTCACCGTCTCCTCA
HCVR (V_{H}) Amino Acid Sequence
(SEQ ID NO: 411)
EVQLVESGGGLVQPGGSLRLSCAASGFTFNIFEMNWVRQAPGKGLEWISYISSRGTTTYYADSVRGRFTISRDNAKN
SLYLQMNSLRAEDTAVYYCARDYEATIPFDFWGQGTLVTVSS
HCDR1:
(SEQ ID NO: 412)
GFTFNIFE
HCDR2:
(SEQ ID NO: 413)
ISSRGTTT
HCDR3:
(SEQ ID NO: 414)
ARDYEATIPFDF
LCVR (V_{L}) Nucleotide Sequence
(SEQ ID NO: 415)
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCAAAGTGGGGTCCCGTCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
LCVR (V_{L}) Amino Acid Sequence
(SEQ ID NO: 416)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIK
LCDR1:
(SEQ ID NO: 417)
QSISSY
LCDR2:
(SEQ ID NO: 418)
AAS
LCDR3:
(SEQ ID NO: 419)
QQSYSTPPIT
12844B
HCVR (V_{H}) Nucleotide Sequence
(SEQ ID NO: 420)
GAGGTGCAGCTGGTGGAGTCTGGGGGAAGTGTGGTACGGCCTGGGGGGTCCCTGAGACTCTCCTGTGAAGCCTCTGG
ATTCACCTTTGATGATTATGGCATGAGCTGGGTCCGCCAAGATCCAGGGAAGGGGCTGGAGTGGGTCTCTGGTATTA
ATTGGAATGGTGATAGAACAAATTATGCAGACTCTGTGAAGGGCCGATTCATCATTTCCAGAGACAACGCCAAGAAC
TCTGTGTATCTACAAATGAACAGTCTGAGAGCGGAGGACTCGGCCTTGTATCACTGTGCGAGAGATCAGGGACTCGG
AGTGGCAGCTACCCTTGACTACTGGGGCCAGGGAACCCTGGTCACCGTCTCCTCA
HCVR (V_{H}) Amino Acid Sequence
(SEQ ID NO: 421)
EVQLVESGGSVVRPGGSLRLSCEASGFTFDDYGMSWVRQDPGKGLEWVSGINWNGDRTNYADSVKGRFIISRDNAKN
SVYLQMNSLRAEDSALYHCARDQGLGVAATLDYWGQGTLVTVSS
or
(SEQ ID NO: 701)
EVQLVESGGSVVRPGGSLRLSCEASGFTFDDYGMSWVRQDPGKGLEWVSGINWNGDRTNYADSVKGRFIISRDNAKN
SVYLQMNSLRAEDSALYHCARDQGLGVAATLDYWGQGTMVTVSS
HCDR1:
(SEQ ID NO: 422)
GFTFDDYG
HCDR2:
(SEQ ID NO: 423)
INWNGDRT
HCDR3:
(SEQ ID NO: 424)
ARDQGLGVAATLDY
LCVR (V_{L}) Nucleotide Sequence
(SEQ ID NO: 425)
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCAAAGTGGGGTCCCGTCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
LCVR (V_{L}) Amino Acid Sequence
(SEQ ID NO: 426)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIK
LCDR1:
(SEQ ID NO: 427)
QSISSY
LCDR2:
(SEQ ID NO: 428)
AAS
LCDR3:
(SEQ ID NO: 429)
QQSYSTPPIT
12845B (REGN17082 Fab; REGN17076 scFv; REGN16825 anti-hTfR scFv:hGAA)
HCVR (V_{H}) Nucleotide Sequence
(SEQ ID NO: 430)
GAGGTGCAGCTGGTGGAGTCTGGGGGAGGCTTGGTACAGCCTGGAGGGTCCCTGAGACTCTCCTGTGCAGCCTCTGG
ATTCACCGTCAGTAATTATGAAATGAACTGGGTCCGCCAGGCTCCAGGGAAGGGGCTGGAGTGGGTTTCATACATTA
GTAGTAGTACCAGTAACATATACTACGCAGACTCTGTGAAGGGCCGATTCACCATCTCCAGAGACAACGCCGAGAAC
TCACTGTATCTGCAGATGAACAGCCTGAGAGTCGAGGACACGGCTGTTTATTACTGTGTGAGAGATGGGATTGTAGT
AGTTCCAGTTGGTCGTGGATACTACTATTACGGTTTGGACGTCTGGGGCCAAGGGACCACGGTCACCGTCTCCTCA
HCVR (V_{H}) Amino Acid Sequence
(SEQ ID NO: 431)
EVQLVESGGGLVQPGGSLRLSCAASGFTVSNYEMNWVRQAPGKGLEWVSYISSSTSNIYYADSVKGRFTISRDNAEN
SLYLQMNSLRVEDTAVYYCVRDGIVVVPVGRGYYYYGLDVWGQGTTVTVSS
HCDR1:
(SEQ ID NO: 432)
GFTVSNYE
HCDR2:
(SEQ ID NO: 433)
ISSSTSNI
HCDR3:
(SEQ ID NO: 434)
VRDGIVVVPVGRGYYYYGLDV
LCVR (V_{L}) Nucleotide Sequence
(SEQ ID NO: 435)
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCAAAGTGGGGTCCCGTCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
LCVR (V_{L}) Amino Acid Sequence
(SEQ ID NO: 436)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIK
LCDR1:
(SEQ ID NO: 437)
QSISSY
LCDR2:
(SEQ ID NO: 438)
AAS
LCDR3:
(SEQ ID NO: 439)
QQSYSTPPIT
12839B (REGN17080 Fab; REGN17074 scFv; REGN16822 anti-hTfR scFv:hGAA)
HCVR (V_{H}) Nucleotide Sequence
(SEQ ID NO: 440)
CAGGTGCAGCTGGTGGAGTCTGGGGGAGGCGTGGTCCAGCCTGGAAGGTCCCTGAGACTCTCCTGCGCAGCCTCTGG
ATTCCCCTTTAGTAATTATGTCATGTATTGGGTCCGCCAGGCTCCAGGCAAGGGGCTGGAGTGGGTGGCTCTTATTT
TTTTTGACGGAAAGAAAAACTATCATGCAGACTCCGTGAAGGGCCGATTCACCATAACCAGAGACAATTCCAAAAAT
ATGTTATATCTGCAAATGAACAGCCTGAGACCTGAGGACGCGGCTGTGTATTACTGTGCGAAAATCCATTGTCCTAA
TGGTGTATGTTACAAGGGGTATTACGGAATGGACGTCTGGGGCCAAGGGACCACGGTCACCGTCTCCTCA
HCVR (V_{H}) Amino Acid Sequence
(SEQ ID NO: 441)
QVQLVESGGGVVQPGRSLRLSCAASGFPFSNYVMYWVRQAPGKGLEWVALIFFDGKKNYHADSVKGRFTITRDNSKN
MLYLQMNSLRPEDAAVYYCAKIHCPNGVCYKGYYGMDVWGQGTTVTVSS
HCDR1:
(SEQ ID NO: 442)
GFPFSNYV
HCDR2:
(SEQ ID NO: 443)
IFFDGKKN
HCDR3:
(SEQ ID NO: 444)
AKIHCPNGVCYKGYYGMDV
LCVR (V_{L}) Nucleotide Sequence
(SEQ ID NO: 445)
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCAAAGTGGGGTCCCGTCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
LCVR (V_{L}) Amino Acid Sequence
(SEQ ID NO: 446)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIK
LCDR1:
(SEQ ID NO: 447)
QSISSY
LCDR2:
(SEQ ID NO: 448)
AAS
LCDR3:
(SEQ ID NO: 449)
QQSYSTPPIT
12841B (REGN16823 anti-hTfR scFv:hGAA)
HCVR (V_{H}) Nucleotide Sequence
(SEQ ID NO: 450)
GAGGTGCAGCTGGTGGAGTCTGGGGGAGGCTTGGTCCAGCCTGGGGGGTCCCTAAGACTCTCCTGTGCAGCCTCTGG
ATTCACCTTTAGTAACTATTGGATGAACTGGGTCCGCCAGGCTCCAGGGAAGGGACTGGAGTGGGTGGCCAATATAA
AAGAAGATGGAGGTAAGAAATTGTATGTGGACTCTGTGAAGGGCCGATTCACCATCTCCAGAGACAACGCCAAGAAC
TCACTGTTTCTGCAAATGAACAGCCTGAGAGCCGAGGACACGGCTGTGTATTATTGTGCGAGAGAAGATACAACTTT
GGTTGTGGACTACTACTACTACGGTATGGACGTCTGGGGCCAAGGGACCACGGTCACCGTCTCCTCA
HCVR (V_{H}) Amino Acid Sequence
(SEQ ID NO: 451)
EVQLVESGGGLVQPGGSLRLSCAASGFTFSNYWMNWVRQAPGKGLEWVANIKEDGGKKLYVDSVKGRFTISRDNAKN
SLFLQMNSLRAEDTAVYYCAREDTTLVVDYYYYGMDVWGQGTTVTVSS
HCDR1:
(SEQ ID NO: 452)
GFTFSNYW
HCDR2:
(SEQ ID NO: 453)
IKEDGGKK
HCDR3:
(SEQ ID NO: 454)
AREDTTLVVDYYYYGMDV
LCVR (V_{L}) Nucleotide Sequence
(SEQ ID NO: 455)
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCAAAGTGGGGTCCCGTCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
LCVR (V_{L}) Amino Acid Sequence
(SEQ ID NO: 456)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIK
LCDR1:
(SEQ ID NO: 457)
QSISSY
LCDR2:
(SEQ ID NO: 458)
AAS
LCDR3:
(SEQ ID NO: 459)
QQSYSTPPIT
12850B (REGN16828 anti-hTfR scFv:hGAA)
HCVR (V_{H}) Nucleotide Sequence
(SEQ ID NO: 460)
CAGGTCCAGCTGGTGCAGTCTGGGGCTGAGGTGAAGAAGCCTGGGTCCTCGGTGAAGGTCTCCTGCAAGGCTTCTGG
AGGCACCTTCAACACCTATGCTATCACCTGGGTGCGACAGGCCCCTGGACAAGGGCTTGAATGGATGGGGGGAATCA
TCCCTATCTCTGGCATAGCAGAGTACGCACAGAAGTTCCAGGGCAGAGTCACGATCACCACGGATGACTCCTCGACC
ACAGCCTACATGGAACTGAACAGTCTGAGATCTGAGGACACGGCCGTGTATTACTGTGCGAGCTGGAACTACGCACT
CTACTACTTCTACGGTATGGACGTCTGGGGCCGAGGGACCACGGTCACCGTCTCCTCA
HCVR (V_{H}) Amino Acid Sequence
(SEQ ID NO: 461)
QVQLVQSGAEVKKPGSSVKVSCKASGGTFNTYAITWVRQAPGQGLEWMGGIIPISGIAEYAQKFQGRVTITTDDSST
TAYMELNSLRSEDTAVYYCASWNYALYYFYGMDVWGRGTTVTVSS
HCDR1:
(SEQ ID NO: 462)
GGTFNTYA
HCDR2:
(SEQ ID NO: 463)
IIPISGIA
HCDR3:
(SEQ ID NO: 464)
ASWNYALYYFYGMDV
LCVR (V_{L}) Nucleotide Sequence
(SEQ ID NO: 465)
GAAATTGTGTTGACGCAGTCTCCAGGCACCCTGTCTTTGTCTCCAGGGGAAAGAGCCACCCTCTCCTGCAGGGCCAG
TCAGAGTGTTAGCAGCAGCTACTTAGCCTGGTACCAGCAGAAACCTGGCCAGGCTCCCAGGCTCCTCATCTATGGTG
CATCCAGCAGGGCCACTGGCATCCCAGACAGGTTCAGTGGCAGTGGGTCTGGGACAGACTTCACTCTCACCATCAGC
AGACTGGAGCCTGAAGATTTTGCAGTGTATTACTGTCAGCAGTATGGTAGCTCACCTTGGACGTTCGGCCAAGGGAC
CAAGGTGGAAATCAAA
LCVR (V_{L}) Amino Acid Sequence
(SEQ ID NO: 466)
EIVLTQSPGTLSLSPGERATLSCRASQSVSSSYLAWYQQKPGQAPRLLIYGASSRATGIPDRFSGSGSGTDFTLTIS
RLEPEDFAVYYCQQYGSSPWTFGQGTKVEIK
LCDR1:
(SEQ ID NO: 467)
QSVSSSY
LCDR2:
(SEQ ID NO: 468)
GAS
LCDR3:
(SEQ ID NO: 469)
QQYGSSPWT
69261
HCVR (V_{H}) Nucleotide Sequence
(SEQ ID NO: 470)
CAGGTGCAGCTGGTGGAGTCTGGGGGAGGCTTGGTCAAGCCTGGAGGGTCCCTGAGACTCTCCTGTGCAGCCTCTGG
ATTCACCTTCAGTGTCTATTACATGAACTGGATCCGCCAGGCTCCAGGGAAGGGCCTGGAGTGGGTTTCATACATTA
GTAGTAGTGGTAGTACCATATACTACGCAGACTCTGTGAAGGGCCGATTCACCATCTCCAGGGACAACGCCAAGAAC
TCACTGTATCTCCAAATGAACAGTCTGAGAGCCGAGGACACGGCCGTATATTACTGTGGGAGAGAAGGGTATAGTGG
GACTTATTCTTATTACGGTATGGACGTCTGGGGCCAAGGGACCACGGTCACCGTCTCCTCA
HCVR (V_{H}) Amino Acid Sequence
(SEQ ID NO: 471)
QVQLVESGGGLVKPGGSLRLSCAASGFTFSVYYMNWIRQAPGKGLEWVSYISSSGSTIYYADSVKGRFTISRDNAKN
SLYLQMNSLRAEDTAVYYCGREGYSGTYSYYGMDVWGQGTTVTVSS
or
(SEQ ID NO: 702)
EVQLVESGGGLVKPGGSLRLSCAASGFTFSVYYMNWIRQAPGKGLEWVSYISSSGSTIYYADSVKGRFTISRDNAKN
SLYLQMNSLRAEDTAVYYCGREGYSGTYSYYGMDVWGQGTTVTVSS
HCDR1:
(SEQ ID NO: 472)
GFTFSVYY
HCDR2:
(SEQ ID NO: 473)
ISSSGSTI
HCDR3:
(SEQ ID NO: 474)
GREGYSGTYSYYGMDV
LCVR (V_{L}) Nucleotide Sequence
(SEQ ID NO: 475)
GATATTGTGATGACTCAGTCTCCACTCTCCCTGCCCGTCACCCCTGGAGAGCCGGCCTCCATCTCCTGCAGGTCTAG
TCAGAGCCTCCTGCATAGTAATGGATACAACTATTTGGATTGGTACCTGCAGAAGCCAGGGCAGTCTCCACAGTTCC
TGATCTATTTGGGTTCTAATCGGGCCTCCGGGGTCCCTGACAGGTTCAGTGGCAGTGGATCAGGCACAGATTTTACA
CTGAAAATCAACAGAGTGGAGGCTGAGGATGTTGGGGTTTATTACTGCATGCAAGCTCTACAAACTCCGTACACTTT
TGGCCAGGGGACCAAGCTGGAGATCAAA
LCVR (V_{L}) Amino Acid Sequence
(SEQ ID NO: 476)
DIVMTQSPLSLPVTPGEPASISCRSSQSLLHSNGYNYLDWYLQKPGQSPQFLIYLGSNRASGVPDRFSGSGSGTDET
LKINRVEAEDVGVYYCMQALQTPYTFGQGTKLEIK
or
(SEQ ID NO: 632)
DIQLTQSPLSLPVTPGEPASISCRSSQSLLHSNGYNYLDWYLQKPGQSPQFLIYLGSNRASGVPDRFSGSGSGTDFT
LKINRVEAEDVGVYYCMQALQTPYTFGQGTKVEIK
LCDR1:
(SEQ ID NO: 477)
QSLLHSNGYNY
LCDR2:
(SEQ ID NO: 478)
LGS
LCDR3:
(SEQ ID NO: 479)
MQALQTPYT
69263
HCVR (V_{H}) Nucleotide Sequence
(SEQ ID NO: 480)
GAAGTGCAGCTGGTGGAGTCTGGGGGAGGGTTGGTACAGCCTGGCAGGTCCCTGAGACTCTCCTGTGCAGTCTCTGG
ATTCACCTTTGATGATTATGCCATGCACTGGGTCCGGCAAGCTCCAGGGAAGGGCCTGGAGTGGGTCTCAGGTATTA
GTTGGAATAGTGGTACCAGAGGATATGCGGACTCTGTGAAGGGCCGATTCACCATCTCCAGAGACAACGCCAAGAAC
TCCCTGTATCTGCAAATGAACAGTCTGAGAGGTGAGGACACGGCCTTGTATTACTGTGTAAAAGATATTACGATATC
CCCCAACTACTACGGTATGGACGTCTGGGGCCAAGGGACCACGGTCACCGTCTCCTCA
HCVR (V_{H}) Amino Acid Sequence
(SEQ ID NO: 481)
EVQLVESGGGLVQPGRSLRLSCAVSGFTEDDYAMHWVRQAPGKGLEWVSGISWNSGTRGYADSVKGRFTISRDNAKN
SLYLQMNSLRGEDTALYYCVKDITISPNYYGMDVWGQGTTVTVSS
HCDR1:
(SEQ ID NO: 482)
GFTFDDYA
HCDR2:
(SEQ ID NO: 483)
ISWNSGTR
HCDR3:
(SEQ ID NO: 484)
VKDITISPNYYGMDV
LCVR (V_{L}) Nucleotide Sequence
(SEQ ID NO: 485)
GACATCCAGATGACCCAGTCTCCATCCTCCCTGTCTGCATCTGTAGGAGACAGAGTCACCATCACTTGCCGGGCGAG
TCAGGACATTAGCCATTATTCAGCCTGGTATCAGCAGAAACCAGGGAAACTTCCTAACCTCCTGATCTATGCTGCAT
CCACTTTGCAATCAGGGGTCCCATCTCGGTTCAGTGGCAGTGGATCTGGGACAGATTTCTCTCTCACCACCAGCAGC
CTGCAGCCTGAAGATGTTGCAACTTATTACTGTCAAAAGTATAACAGTGTCCCTCTCACTTTCGGCGGAGGGACCAA
GGTGGAGATCAAA
LCVR (V_{L}) Amino Acid Sequence
(SEQ ID NO: 486)
DIQMTQSPSSLSASVGDRVTITCRASQDISHYSAWYQQKPGKLPNLLIYAASTLQSGVPSRFSGSGSGTDFSLTTSS
LQPEDVATYYCQKYNSVPLTFGGGTKVEIK
or
(SEQ ID NO: 703)
DIQLTQSPSSLSASVGDRVTITCRASQDISHYSAWYQQKPGKLPNLLIYAASTLQSGVPSRFSGSGSGTDFSLTTSS
LQPEDVATYYCQKYNSVPLTFGGRTKVEIK
LCDR1:
(SEQ ID NO: 487)
QDISHY
LCDR2:
(SEQ ID NO: 488)
AAS
LCDR3:
(SEQ ID NO: 489)
QKYNSVPLT

TABLE 3
Anti-hTfR scFv Molecules in Fusion Proteins.

| | SEQ | |
|---|---|---|
| Antibody | ID | |
| clone | NO | Amino acid sequence (Vk-3xG4S(SEQ ID NO: 616)-Vh) |
| 12795B | 492 | DIQMTQSPSSLSASVGDRVTITCRASQGIRDHFGWYQQKPGKAPKRLIYAASSLHSGVPSRFSGSGS |
| | | GTEFTLTISSLQPEDFATYYCLQYDTYPLTFGGGTKVEIKGGGGSGGGGSGGGGSEVQLVESGGGLV |
| | | QPGGSLRLSCATSGFTFTSYDMKWVRQAPGLGLEWVSAISGSGGNTYYADSVKGRFTISRDNSRNTL |
| | | YLQMNSLRAEDTAVYYCTRSHDFGAFDYFDYWGQGTLVTVSS |
| 12798B | 493 | EIVMTQSPATLSVSPGERATLSCRASQTVSSNLAWYQQKPGQAPRLLIYGSSSRATGIPARFSGSGS |
| | | GTEFTLTISSLQSEDFAVYYCQQYNNWPPYTFGQGTKLEIKGGGGSGGGGSGGGGSEVQLVESGGDL |
| | | VQPGRSLRLSCAASGFTFDDYAMHWVRQAPGKGLEWVSGISWNSATRVYADSVKGRFTISRDNAKNF |
| | | LYLQMNSLRSEDTALYHCAKDMDISLGYYGLDVWGQGTTVTVSS |
| 12799B | 494 | DIQMTQSPSSVSASVGDRVTITCRASQGIASWLAWYQQKPGKAPELLIYAASSLQGGVPSRFSGSGS |
| | | GTDFTLTISSLQPEDFAIYYCQQANYFPWTFGQGTKVEIKGGGGSGGGGSGGGGSQITLKESGPTLV |
| | | KPTQTLTLTCTFSGFSLSTSGVGVVWIRQPPGKALEWLALIYWNDHKRYSPSLGSRLTITKDTSKNQ |
| | | VVLTMTNMDPVDTATYYCAHYSGSYSYYYYGLDVWGQGTTVTVSS |
| 12801B | 495 | DIQMTQSPSSLSASVGDRVTITCRASQGIRTDLGWYQQKPGKAPKRLIYAASSLQSGVPSRFSGSGS |
| | | GTEFTLTISSLRPEDFATFYCLQYNSYPLTFGGGTKVEIKGGGGSGGGGSGGGGSEVQLLESGGALV |
| | | QPGGSLRLSCAASGFTFTSYAMHWVRQAPGKGLEWVSSIRGSGGGTYSADSVKGRFTISRDNSRDTL |
| | | YLQMNSVRAEDTAVYYCARSHDYGAFDFFDYWGQGTLVTVSS |
| 12802B | 496 | EIVMTQSPATLSVSPGERATLSCRASQSVSINLAWYQQKPGQAPRLLIFVASTRATGIPARFSGSGS |
| | | GTEFTLTISSLQSEDFATYYCQQYDIWPYTFGQGTKLEIKGGGGSGGGGSGGGGSQVQLVESGGGLV |
| | | KPGGSLRLSCAASGFTFSDYFMSWIRQAPGKGLEWVSYISSTGSTINYADSVKGRFTISRDNVKNSL |
| | | YLQMTSLRVEDTAVYYCTRDNWNYEYWGQGTLVTVSS |
| 12808B | 497 | DIQMTQSPSSLSASVGDRVTINCRASQGIRNDLGWYQQKPGKAPKRLIYAASSLQSGVPLRFSGSGS |
| | | GTEFTLTINNLQPEDFATYYCLSHNSYPWTFGQGTKVEIKGGGGSGGGGSGGGGSQLQLQESGPGLV |
| | | KPSETLSLTCTVSGESISSNTYYWGWIRQPPGKGLEWIGSIDYSGTTNYNPSLKSRVTISVDTSRNH |
| | | FSLRLRSVTAADTAVYYCAREWGNYGYYYGMDVWGQGTTVTVSS |
| 12812B | 498 | DIQMTQSPPSVSASVGDRVTITCRASQGISSWLAWYQQKPGKAPKLLIYAASSLQSGVPSRFSGSGS |
| | | GTDFTLTISSLQPEDFATYYCQQANSFPRTFGQGTKVEIKGGGGSGGGGSGGGGSQVQLVQSGAEVK |
| | | KPGSSVRVSCKASRGTFSSYAISWVRQAPGQGLEWMGGIIPIFGTANYAQKFLARVTITADESTSTA |
| | | YMELSSLRSEDTAVYYCAREKGWNYFDYWGQGTLVTVSS |
| 12816B | 499 | DIVMTQSPLSLPVTPGEPASISCRSSQSLLHGNGYNYLTWYLQKPGQSPQLLIYLGSNRASGVPDRF |
| | | SGSGSGTDFTLKISRVEAEDVGVYYCMQALQTPYTFGQGTKLEIKGGGGSGGGGSGGGGSQVQLVES |
| | | GGGLVKPGGSLRLSCAASGFTFSDYYMNWIRQAPGKGLEWVSYISSSGTTIYYADSVKGRFTISRDN |
| | | AKKSLYLEMNSLRAEDTAVYYCAREGYGNDYYYYGIDVWGQGTTVTVSS |
| 12833B | 500 | DIQMTQSPSSLSASVGDRVTITCRASQSISSYLNWYQQKPGKAPKLLIYAASSLQSGVPSRFSGSGS |
| | | GTDFTLTISSLQPEDFATYYCQQSYSTPPITFGQGTRLEIKGGGGSGGGGSGGGGSQVQLVESGGGV |
| | | VQPGRSLRLSCAASGFTFSSFGMHWVRQAPGKGLEWVIFISYDGSDKYYADSVKGRFAISRDSSKNT |
| | | LYLQMNSLRAEDTAVYYCAKENGILTDSYGMDVWGQGTTVTVSS |
| 12834B | 501 | DIQMTQSPSSLSASVGDRVTITCRASQSISSYLNWYQQKPGKAPKLLIYAASSLQSGVPSRFSGSGS |
| | | GTDFTLTISSLQPEDFATYYCQQSYSTPPITFGQGTRLEIKGGGGSGGGGSGGGGSQVQLVQSGAEV |
| | | KKPGASVKVSCKASGYTFTSYGISWVRQAPGQGLEWMGWISVYHGNTNYAQKFQGRVTMTTDTSTST |
| | | AYMELRSLRSDDTAVYYCAREGYYDFWSGYYPFDYWGQGTLVTVSS |
| 12835B | 502 | DIQMTQSPSSLSASVGDRVTITCRASQSISSYLNWYQQKPGKAPKLLIYAASSLQSGVPSRFSGSGS |
| | | GTDFTLTISSLQPEDFATYYCQQSYSTPPITFGQGTRLEIKGGGGSGGGGSGGGGSEVQLVESGGGL |
| | | IQPGGSLRLSCEASGFTFRNYEMNWVRQAPGKGLEWVSYISSSGNMKDYAESVKGRFTISRDNVKNS |
| | | LQLQMNSLRVEDTAVYYCARDEFPYGMDVWGQGTTVTVSS |
| 12839B | 503 | DIQMTQSPSSLSASVGDRVTITCRASQSISSYLNWYQQKPGKAPKLLIYAASSLQSGVPSRFSGSGS |
| | | GTDFTLTISSLQPEDFATYYCQQSYSTPPITFGQGTRLEIKGGGGSGGGGSGGGGSQVQLVESGGGV |
| | | VQPGRSLRLSCAASGFPFSNYVMYWVRQAPGKGLEWVALIFFDGKKNYHADSVKGRFTITRDNSKNM |
| | | LYLQMNSLRPEDAAVYYCAKIHCPNGVCYKGYYGMDVWGQGTTVTVSS |
| 12841B | 504 | DIQMTQSPSSLSASVGDRVTITCRASQSISSYLNWYQQKPGKAPKLLIYAASSLQSGVPSRFSGSGS |
| | | GTDFTLTISSLQPEDFATYYCQQSYSTPPITFGQGTRLEIKGGGGSGGGGSGGGGSEVQLVESGGGL |
| | | VQPGGSLRLSCAASGFTFSNYWMNWVRQAPGKGLEWVANIKEDGGKKLYVDSVKGRFTISRDNAKNS |
| | | LFLQMNSLRAEDTAVYYCAREDTTLVVDYYYYGMDVWGQGTTVTVSS |
| 12843B | 505 | DIQMTQSPSSLSASVGDRVTITCRASQSISSYLNWYQQKPGKAPKLLIYAASSLQSGVPSRFSGSGS |
| | | GTDFTLTISSLQPEDFATYYCQQSYSTPPITFGQGTRLEIKGGGGSGGGGSGGGGSEVQLVESGGGL |
| | | VQPGGSLRLSCAASGFTFNIFEMNWVRQAPGKGLEWISYISSRGTTTYYADSVRGRFTISRDNAKNS |
| | | LYLQMNSLRAEDTAVYYCARDYEATIPFDFWGQGTLVTVSS |
| 12844B | 506 | DIQMTQSPSSLSASVGDRVTITCRASQSISSYLNWYQQKPGKAPKLLIYAASSLQSGVPSRFSGSGS |
| | | GTDFTLTISSLQPEDFATYYCQQSYSTPPITFGQGTRLEIKGGGGSGGGGSGGGGSEVQLVESGGSV |
| | | VRPGGSLRLSCEASGFTFDDYGMSWVRQDPGKGLEWVSGINWNGDRTNYADSVKGRFIISRDNAKNS |
| | | VYLQMNSLRAEDSALYHCARDQGLGVAATLDYWGQGTLVTVSS |
| 12845B | 507 | DIQMTQSPSSLSASVGDRVTITCRASQSISSYLNWYQQKPGKAPKLLIYAASSLQSGVPSRFSGSGS |
| | | GTDFTLTISSLQPEDFATYYCQQSYSTPPITFGQGTRLEIKGGGGSGGGGSGGGGSEVQLVESGGGL |
| | | VQPGGSLRLSCAASGFTVSNYEMNWVRQAPGKGLEWVSYISSSTSNIYYADSVKGRFTISRDNAENS |
| | | LYLQMNSLRVEDTAVYYCVRDGIVVVPVGRGYYYYGLDVWGQGTTVTVSS |
| 12847B | 508 | DIQMTQSPSSLSASVGDRVTITCRASQSISSYLNWYQQKPGKAPKLLIYAASSLQSGVPSRFSGSGS |
| | | GTDFTLTISSLQPEDFATYYCQQSYSTPPITFGQGTRLEIKGGGGSGGGGSGGGGSEVQLVESGGGL |
| | | VQPGRSLRLSCAASGFTFDDYAMNWVRQAPGKGLEWVSGISWSSGSMDYADSVKGRFTISRDNAKNS |
| | | LYLQMNSLRTEDTALYYCAKAREVGDYYGMDVWGQGTTVTVSS |
| 12848B | 509 | EIVLTQSPGTLSLSPGERATLSCRASQSVSSSYLAWYQQKPGQAPRLLIYGASSRATGIPDRFSGSG |
| | | SGTDFTLTISRLEPEDFAVYYCQQYGSSPWTFGQGTKVEIKGGGGSGGGGSGGGGSEVQLVESGGGL |
| | | VQPGRSLTLSCAASGFTFDNFGMHWVRQGPGKGLEWVSGLTWNSGVIGYADSVKGRFTISRDNAKNS |
| | | LYLQMNSLRPEDTALYYCAKDIRNYGPFDYWGQGTLVTVSS |
| 12850B | 510 | EIVLTQSPGTLSLSPGERATLSCRASQSVSSSYLAWYQQKPGQAPRLLIYGASSRATGIPDRFSGSG |
| | | SGTDFTLTISRLEPEDFAVYYCQQYGSSPWTFGQGTKVEIKGGGGSGGGGSGGGGSQVQLVQSGAEV |
| | | KKPGSSVKVSCKASGGTFNTYAITWVRQAPGQGLEWMGGIIPISGIAEYAQKFQGRVTITTDDSSTT |
| | | AYMELNSLRSEDTAVYYCASWNYALYYFYGMDVWGRGTTVTVSS |
| 31863B | 511 | DIQMTQSPSSLSASIGDRVTITCRASQGISNYLAWYQQKPGKVPKLLIYAASTLQSGVPSRFSGSGS |
| | | GTDFTLTISSLQPEDVATYYCQNHNSVPLTFGGGTKVEIKGGGGSGGGGSGGGGSEVQLVESGGGLV |
| | | QPGGSLRLSCAASGFTFNSYAMTWVRQAPGKGLEWVSFIGGSTGNTYYAGSVKGRFTISSDNSKKTL |
| | | YLQMNSLRAEDTAVYYCAKGGAARRMEYFQHWGQGTLVTVSS |
| 31874B | 512 | DIQMTQSPSSLSASVGDRVTITCRASQGISNYLAWYQQKPGKVPNLLIYAASTLQSGVPSRFSGSGS |
| | | GTDFTLTISSLQPEDVATYYCQKYNSAPLTFGGGTKVEIKGGGGSGGGGSGGGGSEVQLVESGGGLV |
| | | QPGGSLRLSCAASGFAFSSYAMTWVRQAPGKGLEWVSVISGTGGSTYYADSVKGRFTISRDNSKNTL |
| | | YLQMNSLRAEDTAVYYCAKGGAARRMEYFQYWGQGTLVTVSS |
| 69261 | 513 | DIVMTQSPLSLPVTPGEPASISCRSSQSLLHSNGYNYLDWYLQKPGQSPQFLIYLGSNRASGVPDRF |
| | | SGSGSGTDFTLKINRVEAEDVGVYYCMQALQTPYTFGQGTKLEIKGGGGSGGGGSGGGGSQVQLVES |
| | | GGGLVKPGGSLRLSCAASGFTFSVYYMNWIRQAPGKGLEWVSYISSSGSTIYYADSVKGRFTISRDN |
| | | AKNSLYLQMNSLRAEDTAVYYCGREGYSGTYSYYGMDVWGQGTTVTVSS |
| 69263 | 514 | DIQMTQSPSSLSASVGDRVTITCRASQDISHYSAWYQQKPGKLPNLLIYAASTLQSGVPSRFSGSGS |
| | | GTDFSLTTSSLQPEDVATYYCQKYNSVPLTFGGGTKVEIKGGGGSGGGGSGGGGSEVQLVESGGGLV |
| | | QPGRSLRLSCAVSGFTFDDYAMHWVRQAPGKGLEWVSGISWNSGTRGYADSVKGRFTISRDNAKNSL |
| | | YLQMNSLRGEDTALYYCVKDITISPNYYGMDVWGQGTTVTVSS |
| 69305 | 515 | DIQMTQSPSSLSASVGDRVTITCRASQSIDRYLNWYRQKPGKAPKLLIYTTSSLQSGVPSRFSGSGS |
| | | GTDFTLTLSSLQPEDFATYYCQQSYSPPLTFGGGTKVEIKGGGGSGGGGSGGGGSQVQLVESGGGVV |
| | | QPGRSLRLSCAASGFTFSSYGMHWVRQAPGKGLEWVAVIWYDGSNKYYADSVKGRFTISRDISKNTL |
| | | YLQMNSLRAEDTAVYYCAGQLDLFFDYWGQGTLVTVSS |
| 69307 | 516 | DIQMTQSPSSVSASVGDRVTITCRASQGISSWLAWYQQKPGKAPKLLIYAASSLQSGVPSRFSGSGS |
| | | GTDFTLTISSLQPEDFATYYCQKADSLPYAFGQGTKLEIKGGGGSGGGGSGGGGSEVQLVESGGGLV |
| | | QPGGSLRLSCTASGFTFSNYWMTWVRQAPGKGLEWVANIKEDGSEKEYVDSVKGRFTISRDNAKNSL |
| | | YLQMNSLRGEDTAVYYCARDGEQLVDYYYYYVMDVWGQGTTVTVSS |
| 69323 | 517 | DIQMTQSPSSLSASVGDRVTITCRASQSISSYLNWYQQKPGKAPKVLIYAASSLQSGVPSRFSGSGS |
| | | GTDFTLTISSLQPEDFATYYCQQSYSIPLTFGGGTKVEIKGGGGSGGGGSGGGGSEVQLVESGGGLV |
| | | QPGRSLRLSCAASGFTFDDYAMHWVRQAPGKGLEWVSGISWNSGYIGYADSVKGRFTISRDNAENSL |
| | | HLQMNSLRAEDTALYYCARGGSTLVRGVKGGYYGMDVWGQGTTVTVSS |
| 69326 | 518 | EIVMTQSPATLSVSPGERATLSCRASQSVSSNFAWYQQKPGQAPRLLIYSASSRATGIPVRFSGSGS |
| | | GTEFTLTISSLQSEDFAVYYCQQYNIWPRTFGQGTKVEIKGGGGSGGGGSGGGGSEVQLVESGGGLV |
| | | QPGGSLRLSCAVSGFIFSSYEMNWVRQAPGKGLEWVSYISSSGSTIFYADSVKGRFTISRDNAKNSL |
| | | YLQMNSLRAEDTAVYYCVSGVVLFDVWGQGTMVTVSS |
| 69329 | 519 | DIQMTQSPSSVSASVGDRVTITCRASQGISSWLAWYQQKPGKAPKLLIYAASSLQSGVPSRFSGSGS |
| | | GTDFTLTISSLQPEDFATYYCQKANSFPYTFGQGTKLEIKGGGGSGGGGSGGGGSEVQLVESGGGLV |
| | | QPGGSLRLSCAASGFTFSNYWMTWVRQAPGKGLEWVANIKEDGSEKDYVDSVKGRFTISRDNAKNSL |
| | | YLQMNSLRGEDTAVYYCARDGEQLVDYYYYYVMDVWGQGTTVTVSS |
| 69331 | 520 | DIQLTQSPSSLSASVGDRVTITCWASQGISSYLAWYQQKPGKAPKLLIYAASTLQSGVPSRFSGSGS |
| | | GTEFTLTISSLQPEDFATYYCQQLNSYPLTFGGGTKVEIKGGGGSGGGGSGGGGSQVQLVESGGGVV |
| | | QPGRSLRLSCIASGFTFSVYGIHWVRQAPGKGLEWMAVISHDGNIKHYADSVKGRFTISRDNSKNTL |
| | | YLQINSLRTEDTAVYYCAKDTWNSLDTFDIWGQGTMVTVSS |
| 69332 | 521 | AIQMTQSPSSLSASVGDRVTITCRASQGIRNDLGWYQQKPGKAPKLLIYAASTLQSGVPSRFSGSGS |
| | | GTDFTLTISSLQPEDFATYYCLQDYNYPFTFGPGTKVDIKGGGGSGGGGSGGGGSQVTLRESGPALV |
| | | KPSQTLTLTCTFSGFSLNTYGMFVSWIRQPPGKALEWLAHIHWDDDKYYSTSLKTRLTISKDTSKNQ |
| | | VVLTMTNMDPVDTATYYCARGHNNLNYIIHWGQGTLVTVSS |
| 69340 | 522 | EIVLTQSPATLSLSPGERATLSCRASQSVSSYLAWYQQKPGQAPRLLIHDVSNRATGIPARFSGSGS |
| | | GTDFTLTISSLEPEDFVVYYCQQRSDWPITFGQGTRLEIKGGGGSGGGGSGGGGSEVQLVESGGGLV |
| | | QPGRSLRLSCAASGFTFDDKAMHWVRQVPGKGLEWISGISWNSGTIGYADSVKGRFIISRDNAKNSL |
| | | YLQMNSLRAEDTALYYCAKDGDTSGWYWYGLDVWGQGTTVTVSS |
| 69348 | 523 | DIQMTQSPSSLSASVGDRVTITCRASQSIRNVLGWFQQKPGKAPQRLIYAASSLQSGVPSRFSGSGS |
| | | GTEFTLTISSLQPEDFATYYCLQHNFYPLTFGGGTKVEIKGGGGSGGGGSGGGGSQVQLVESGGGVV |
| | | QPGRSLRLSCAASGFTFTTYGMHWVRQAPGKGLEWVAVIWYDGSNKYYGDSVKGRFTISRDNSKNTL |
| | | YLQMNSLRVDDTAVYYCTRTHGYTRSSDGFDYWGQGTLVTVSS |

Heavy and Light Chains of anti-hTfR Fabs in anti-hTfR:GAA Fusion Proteins:
(1) 31874B
Fab Light Chain
(SEQ ID NO: 540)
DIQMTQSPSSLSASVGDRVTITCRASQGISNYLAWYQQKPGKVPNLLIYAASTLQSGVPSRFSGSGSGTDFTLTISS
LQPEDVATYYCQKYNSAPLTFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 541)
EVQLVESGGGLVQPGGSLRLSCAASGFAFSSYAMTWVRQAPGKGLEWVSVISGTGGSTYYADSVKGRFTISRDNSKN
TLYLQMNSLRAEDTAVYYCAKGGAARRMEYFQYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTH
(2) 31863B
Fab Light Chain
(SEQ ID NO: 542)
DIQMTQSPSSLSASIGDRVTITCRASQGISNYLAWYQQKPGKVPKLLIYAASTLQSGVPSRFSGSGSGTDFTLTISS
LQPEDVATYYCQNHNSVPLTFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 543)
EVQLVESGGGLVQPGGSLRLSCAASGFTFNSYAMTWVRQAPGKGLEWVSFIGGSTGNTYYAGSVKGRFTISSDNSKK
TLYLQMNSLRAEDTAVYYCAKGGAARRMEYFQHWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTH
(3) 69348
Fab Light Chain
(SEQ ID NO: 544)
DIQMTQSPSSLSASVGDRVTITCRASQSIRNVLGWFQQKPGKAPQRLIYAASSLQSGVPSRFSGSGSGTEFTLTISS
LQPEDFATYYCLQHNFYPLTFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 545)
QVQLVESGGGVVQPGRSLRLSCAASGFTFTTYGMHWVRQAPGKGLEWVAVIWYDGSNKYYGDSVKGRFTISRDNSKN
TLYLQMNSLRVDDTAVYYCTRTHGYTRSSDGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
(4) 69340
Fab Light Chain
(SEQ ID NO: 546)
EIVLTQSPATLSLSPGERATLSCRASQSVSSYLAWYQQKPGQAPRLLIHDVSNRATGIPARFSGSGSGTDFTLTISS
LEPEDFVVYYCQQRSDWPITFGQGTRLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 547)
EVQLVESGGGLVQPGRSLRLSCAASGFTFDDKAMHWVRQVPGKGLEWISGISWNSGTIGYADSVKGRFIISRDNAKN
SLYLQMNSLRAEDTALYYCAKDGDTSGWYWYGLDVWGQGTTVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKTH
(5) 69331
Fab Light Chain
(SEQ ID NO: 548)
DIQLTQSPSSLSASVGDRVTITCWASQGISSYLAWYQQKPGKAPKLLIYAASTLQSGVPSRFSGSGSGTEFTLTISS
LQPEDFATYYCQQLNSYPLTFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 549)
QVQLVESGGGVVQPGRSLRLSCIASGFTFSVYGIHWVRQAPGKGLEWMAVISHDGNIKHYADSVKGRFTISRDNSKN
TLYLQINSLRTEDTAVYYCAKDTWNSLDTFDIWGQGTMVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTH
(6) 69332
Fab Light Chain
(SEQ ID NO: 550)
AIQMTQSPSSLSASVGDRVTITCRASQGIRNDLGWYQQKPGKAPKLLIYAASTLQSGVPSRFSGSGSGTDFTLTISS
LQPEDFATYYCLQDYNYPFTFGPGTKVDIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 551)
QVTLRESGPALVKPSQTLTLTCTFSGFSLNTYGMFVSWIRQPPGKALEWLAHIHWDDDKYYSTSLKTRLTISKDTSK
NQVVLTMTNMDPVDTATYYCARGHNNLNYIIHWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTH
(7) 69326
Fab Light Chain
(SEQ ID NO: 552)
EIVMTQSPATLSVSPGERATLSCRASQSVSSNFAWYQQKPGQAPRLLIYSASSRATGIPVRFSGSGSGTEFTLTISS
LQSEDFAVYYCQQYNIWPRTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 553)
EVQLVESGGGLVQPGGSLRLSCAVSGFIFSSYEMNWVRQAPGKGLEWVSYISSSGSTIFYADSVKGRFTISRDNAKN
SLYLQMNSLRAEDTAVYYCVSGVVLFDVWGQGTMVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSCDKTH
(8) 69329
Fab Light Chain
(SEQ ID NO: 554)
DIQMTQSPSSVSASVGDRVTITCRASQGISSWLAWYQQKPGKAPKLLIYAASSLQSGVPSRFSGSGSGTDFTLTISS
LQPEDFATYYCQKANSFPYTFGQGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 555)
EVQLVESGGGLVQPGGSLRLSCAASGFTFSNYWMTWVRQAPGKGLEWVANIKEDGSEKDYVDSVKGRFTISRDNAKN
SLYLQMNSLRGEDTAVYYCARDGEQLVDYYYYYVMDVWGQGTTVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCDKT
H
(9) 69323
Fab Light Chain
(SEQ ID NO: 556)
DIQMTQSPSSLSASVGDRVTITCRASQSISSYLNWYQQKPGKAPKVLIYAASSLQSGVPSRFSGSGSGTDFTLTISS
LQPEDFATYYCQQSYSIPLTFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 557)
EVQLVESGGGLVQPGRSLRLSCAASGFTFDDYAMHWVRQAPGKGLEWVSGISWNSGYIGYADSVKGRFTISRDNAEN
SLHLQMNSLRAEDTALYYCARGGSTLVRGVKGGYYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKKVEPKSCD
KTH
(10) 69305
Fab Light Chain
(SEQ ID NO: 558)
DIQMTQSPSSLSASVGDRVTITCRASQSIDRYLNWYRQKPGKAPKLLIYTTSSLQSGVPSRFSGSGSGTDFTLTLSS
LQPEDFATYYCQQSYSPPLTFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 559)
QVQLVESGGGVVQPGRSLRLSCAASGFTFSSYGMHWVRQAPGKGLEWVAVIWYDGSNKYYADSVKGRFTISRDISKN
TLYLQMNSLRAEDTAVYYCAGQLDLFFDYWGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKVDKKVEPKSCDKTH
(11) 69307
Fab Light Chain
(SEQ ID NO: 560)
DIQMTQSPSSVSASVGDRVTITCRASQGISSWLAWYQQKPGKAPKLLIYAASSLQSGVPSRFSGSGSGTDFTLTISS
LQPEDFATYYCQKADSLPYAFGQGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 561)
EVQLVESGGGLVQPGGSLRLSCTASGFTFSNYWMTWVRQAPGKGLEWVANIKEDGSEKEYVDSVKGRFTISRDNAKN
SLYLQMNSLRGEDTAVYYCARDGEQLVDYYYYYVMDVWGQGTTVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCDKT
H
(12) 12795B
Fab Light Chain
(SEQ ID NO: 562)
DIQMTQSPSSLSASVGDRVTITCRASQGIRDHFGWYQQKPGKAPKRLIYAASSLHSGVPSRFSGSGSGTEFTLTISS
LQPEDFATYYCLQYDTYPLTFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 563)
EVQLVESGGGLVQPGGSLRLSCATSGFTFTSYDMKWVRQAPGLGLEWVSAISGSGGNTYYADSVKGRFTISRDNSRN
TLYLQMNSLRAEDTAVYYCTRSHDFGAFDYFDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTH
(13) 12798B (REGN17078)
Fab Light Chain
(SEQ ID NO: 564)
EIVMTQSPATLSVSPGERATLSCRASQTVSSNLAWYQQKPGQAPRLLIYGSSSRATGIPARFSGSGSGTEFTLTISS
LQSEDFAVYYCQQYNNWPPYTFGQGTKLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC
Fab Heavy Chain
(SEQ ID NO: 565)
EVQLVESGGDLVQPGRSLRLSCAASGFTFDDYAMHWVRQAPGKGLEWVSGISWNSATRVYADSVKGRFTISRDNAKN
FLYLQMNSLRSEDTALYHCAKDMDISLGYYGLDVWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH;
or
(SEQ ID NO: 604)
EVQLVESGGDLVQPGRSLRLSCAASGFTFDDYAMHWVRQAPGKGLEWVSGISWNSATRVYADSVKGRFTISRDNAKN
FLYLQMNSLRSEDTALYHCAKDMDISLGYYGLDVWGQGTTVTVSSASTKGPSVFPLAPCSRSTSESTAALGCLVKDY
FPEPVTVSWNSGALTSGVHTFPAVLQSSGLYSLSSVVTVPSSSLGTKTYTCNVDHKPSNTKVDKRVESKYGPPLLQG
SG;
or
(SEQ ID NO: 633)
EVQLVESGGDLVQPGRSLRLSCAASGFTFDDYAMHWVRQAPGKGLEWVSGISWNSATRVYADSVKGRFTISRDNAKN
FLYLQMNSLRSEDTALYHCAKDMDISLGYYGLDVWGQGTTVTVSSASTKGPSVFPLAPCSRSTSESTAALGCLVKDY
FPEPVTVSWNSGALTSGVHTFPAVLQSSGLYSLSSVVTVPSSSLGTKTYTCNVDHKPSNTKVDKRVESKYGPP
(14) 12799B (REGN17079)
Fab Light Chain
(SEQ ID NO: 566)
DIQMTQSPSSVSASVGDRVTITCRASQGIASWLAWYQQKPGKAPELLIYAASSLQGGVPSRFSGSGSGTDFTLTISS
LQPEDFAIYYCQQANYFPWTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 567)
QITLKESGPTLVKPTQTLTLTCTFSGFSLSTSGVGVVWIRQPPGKALEWLALIYWNDHKRYSPSLGSRLTITKDTSK
NQVVLTMTNMDPVDTATYYCAHYSGSYSYYYYGLDVWGQGTTVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSCDKTH;
or
(SEQ ID NO: 605)
QITLKESGPTLVKPTQTLTLTCTFSGFSLSTSGVGVVWIRQPPGKALEWLALIYWNDHKRYSPSLGSRLTITKDTSK
NQVVLTMTNMDPVDTATYYCAHYSGSYSYYYYGLDVWGQGTTVTVSSASTKGPSVFPLAPCSRSTSESTAALGCLVK
DYFPEPVTVSWNSGALTSGVHTFPAVLQSSGLYSLSSVVTVPSSSLGTKTYTCNVDHKPSNTKVDKRVESKYGPPLL
QGSG;
or
(SEQ ID NO: 634)
QITLKESGPTLVKPTQTLTLTCTFSGFSLSTSGVGVVWIRQPPGKALEWLALIYWNDHKRYSPSLGSRLTITKDTSK
NQVVLTMTNMDPVDTATYYCAHYSGSYSYYYYGLDVWGQGTTVTVSSASTKGPSVFPLAPCSRSTSESTAALGCLVK
DYFPEPVTVSWNSGALTSGVHTFPAVLQSSGLYSLSSVVTVPSSSLGTKTYTCNVDHKPSNTKVDKRVESKYGPP
(15) 12801B
Fab Light Chain
(SEQ ID NO: 568)
DIQMTQSPSSLSASVGDRVTITCRASQGIRTDLGWYQQKPGKAPKRLIYAASSLQSGVPSRFSGSGSGTEFTLTISS
LRPEDFATFYCLQYNSYPLTFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 569)
EVQLLESGGALVQPGGSLRLSCAASGFTFTSYAMHWVRQAPGKGLEWVSSIRGSGGGTYSADSVKGRFTISRDNSRD
TLYLQMNSVRAEDTAVYYCARSHDYGAFDFFDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTH
(16) 12802B
Fab Light Chain
(SEQ ID NO: 570)
EIVMTQSPATLSVSPGERATLSCRASQSVSINLAWYQQKPGQAPRLLIFVASTRATGIPARFSGSGSGTEFTLTISS
LQSEDFATYYCQQYDIWPYTFGQGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 571)
QVQLVESGGGLVKPGGSLRLSCAASGFTFSDYFMSWIRQAPGKGLEWVSYISSTGSTINYADSVKGRFTISRDNVKN
SLYLQMTSLRVEDTAVYYCTRDNWNYEYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSCDKTH
(17) 12808B
Fab Light Chain
(SEQ ID NO: 572)
DIQMTQSPSSLSASVGDRVTINCRASQGIRNDLGWYQQKPGKAPKRLIYAASSLQSGVPLRFSGSGSGTEFTLTINN
LQPEDFATYYCLSHNSYPWTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 573)
QLQLQESGPGLVKPSETLSLTCTVSGESISSNTYYWGWIRQPPGKGLEWIGSIDYSGTTNYNPSLKSRVTISVDTSR
NHFSLRLRSVTAADTAVYYCAREWGNYGYYYGMDVWGQGTTVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKTH
(18) 12812B
Fab Light Chain
(SEQ ID NO: 574)
DIQMTQSPPSVSASVGDRVTITCRASQGISSWLAWYQQKPGKAPKLLIYAASSLQSGVPSRFSGSGSGTDFTLTISS
LQPEDFATYYCQQANSFPRTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 575)
QVQLVQSGAEVKKPGSSVRVSCKASRGTESSYAISWVRQAPGQGLEWMGGIIPIFGTANYAQKFLARVTITADESTS
TAYMELSSLRSEDTAVYYCAREKGWNYFDYWGQGTLVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKVDKKVEPKSCDKTH
(19) 12816B
Fab Light Chain
(SEQ ID NO: 576)
DIVMTQSPLSLPVTPGEPASISCRSSQSLLHGNGYNYLTWYLQKPGQSPQLLIYLGSNRASGVPDRFSGSGSGTDFT
LKISRVEAEDVGVYYCMQALQTPYTFGQGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGEC
Fab Heavy Chain
(SEQ ID NO: 577)
QVQLVESGGGLVKPGGSLRLSCAASGFTFSDYYMNWIRQAPGKGLEWVSYISSSGTTIYYADSVKGRFTISRDNAKK
SLYLEMNSLRAEDTAVYYCAREGYGNDYYYYGIDVWGQGTTVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKTH
(20) 12833B
Fab Light Chain
(SEQ ID NO: 578)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC
Fab Heavy Chain
(SEQ ID NO: 579)
QVQLVESGGGVVQPGRSLRLSCAASGFTFSSFGMHWVRQAPGKGLEWVIFISYDGSDKYYADSVKGRFAISRDSSKN
TLYLQMNSLRAEDTAVYYCAKENGILTDSYGMDVWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
(21) 12834B
Fab Light Chain
(SEQ ID NO: 580)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC
Fab Heavy Chain
(SEQ ID NO: 581)
QVQLVQSGAEVKKPGASVKVSCKASGYTFTSYGISWVRQAPGQGLEWMGWISVYHGNTNYAQKFQGRVTMTTDTSTS
TAYMELRSLRSDDTAVYYCAREGYYDFWSGYYPFDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSCDKTH
(22) 12835B
Fab Light Chain
(SEQ ID NO: 582)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC
Fab Heavy Chain
(SEQ ID NO: 583)
EVQLVESGGGLIQPGGSLRLSCEASGFTFRNYEMNWVRQAPGKGLEWVSYISSSGNMKDYAESVKGRFTISRDNVKN
SLQLQMNSLRVEDTAVYYCARDEFPYGMDVWGQGTTVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKVDKKVEPKSCDKTH
(23) 12847B (REGN17083)
Fab Light Chain
(SEQ ID NO: 584)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC
Fab Heavy Chain
(SEQ ID NO: 585)
EVQLVESGGGLVQPGRSLRLSCAASGFTFDDYAMNWVRQAPGKGLEWVSGISWSSGSMDYADSVKGRFTISRDNAKN
SLYLQMNSLRTEDTALYYCAKAREVGDYYGMDVWGQGTTVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTH;
or
(SEQ ID NO: 606)
EVQLVESGGGLVQPGRSLRLSCAASGFTFDDYAMNWVRQAPGKGLEWVSGISWSSGSMDYADSVKGRFTISRDNAKN
SLYLQMNSLRTEDTALYYCAKAREVGDYYGMDVWGQGTTVTVSSASTKGPSVFPLAPCSRSTSESTAALGCLVKDYF
PEPVTVSWNSGALTSGVHTFPAVLQSSGLYSLSSVVTVPSSSLGTKTYTCNVDHKPSNTKVDKRVESKYGPPLLQGS
G;
or
(SEQ ID NO: 635)
EVQLVESGGGLVQPGRSLRLSCAASGFTFDDYAMNWVRQAPGKGLEWVSGISWSSGSMDYADSVKGRFTISRDNAKN
SLYLQMNSLRTEDTALYYCAKAREVGDYYGMDVWGQGTTVTVSSASTKGPSVFPLAPCSRSTSESTAALGCLVKDYF
PEPVTVSWNSGALTSGVHTFPAVLQSSGLYSLSSVVTVPSSSLGTKTYTCNVDHKPSNTKVDKRVESKYGPP
(24) 12848B
Fab Light Chain
(SEQ ID NO: 586)
EIVLTQSPGTLSLSPGERATLSCRASQSVSSSYLAWYQQKPGQAPRLLIYGASSRATGIPDRFSGSGSGTDFTLTIS
RLEPEDFAVYYCQQYGSSPWTFGQGTKVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC
Fab Heavy Chain
(SEQ ID NO: 587)
EVQLVESGGGLVQPGRSLTLSCAASGFTFDNFGMHWVRQGPGKGLEWVSGLTWNSGVIGYADSVKGRFTISRDNAKN
SLYLQMNSLRPEDTALYYCAKDIRNYGPFDYWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVDKKVEPKSCDKTH
(25) 12843B (REGN17081)
Fab Light Chain
(SEQ ID NO: 588)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC
Fab Heavy Chain
(SEQ ID NO: 589)
EVQLVESGGGLVQPGGSLRLSCAASGFTFNIFEMNWVRQAPGKGLEWISYISSRGTTTYYADSVRGRFTISRDNAKN
SLYLQMNSLRAEDTAVYYCARDYEATIPFDFWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVDKKVEPKSCDKTH;
or
(SEQ ID NO: 607)
EVQLVESGGGLVQPGGSLRLSCAASGFTFNIFEMNWVRQAPGKGLEWISYISSRGTTTYYADSVRGRFTISRDNAKN
SLYLQMNSLRAEDTAVYYCARDYEATIPFDFWGQGTLVTVSSASTKGPSVFPLAPCSRSTSESTAALGCLVKDYFPE
PVTVSWNSGALTSGVHTFPAVLQSSGLYSLSSVVTVPSSSLGTKTYTCNVDHKPSNTKVDKRVESKYGPPLLQGSG;
or
(SEQ ID NO: 636)
EVQLVESGGGLVQPGGSLRLSCAASGFTFNIFEMNWVRQAPGKGLEWISYISSRGTTTYYADSVRGRFTISRDNAKN
SLYLQMNSLRAEDTAVYYCARDYEATIPFDFWGQGTLVTVSSASTKGPSVFPLAPCSRSTSESTAALGCLVKDYFPE
PVTVSWNSGALTSGVHTFPAVLQSSGLYSLSSVVTVPSSSLGTKTYTCNVDHKPSNTKVDKRVESKYGPP
(26) 12844B
Fab Light Chain
(SEQ ID NO: 590)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC
Fab Heavy Chain
(SEQ ID NO: 591)
EVQLVESGGSVVRPGGSLRLSCEASGFTFDDYGMSWVRQDPGKGLEWVSGINWNGDRTNYADSVKGRFIISRDNAKN
SVYLQMNSLRAEDSALYHCARDQGLGVAATLDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTH
(27) 12845B (REGN17082)
Fab Light Chain
(SEQ ID NO: 592)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC
Fab Heavy Chain
(SEQ ID NO: 593)
EVQLVESGGGLVQPGGSLRLSCAASGFTVSNYEMNWVRQAPGKGLEWVSYISSSTSNIYYADSVKGRFTISRDNAEN
SLYLQMNSLRVEDTAVYYCVRDGIVVVPVGRGYYYYGLDVWGQGTTVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVEPKSC
DKTH;
or
(SEQ ID NO: 608)
EVQLVESGGGLVQPGGSLRLSCAASGFTVSNYEMNWVRQAPGKGLEWVSYISSSTSNIYYADSVKGRFTISRDNAEN
SLYLQMNSLRVEDTAVYYCVRDGIVVVPVGRGYYYYGLDVWGQGTTVTVSSASTKGPSVFPLAPCSRSTSESTAALG
CLVKDYFPEPVTVSWNSGALTSGVHTFPAVLQSSGLYSLSSVVTVPSSSLGTKTYTCNVDHKPSNTKVDKRVESKYG
PPLLQGSG;
or
(SEQ ID NO: 637)
EVQLVESGGGLVQPGGSLRLSCAASGFTVSNYEMNWVRQAPGKGLEWVSYISSSTSNIYYADSVKGRFTISRDNAEN
SLYLQMNSLRVEDTAVYYCVRDGIVVVPVGRGYYYYGLDVWGQGTTVTVSSASTKGPSVFPLAPCSRSTSESTAALG
CLVKDYFPEPVTVSWNSGALTSGVHTFPAVLQSSGLYSLSSVVTVPSSSLGTKTYTCNVDHKPSNTKVDKRVESKYG
PP
(28) 12839B (REGN17080)
Fab Light Chain
(SEQ ID NO: 594)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC
Fab Heavy Chain
(SEQ ID NO: 595)
QVQLVESGGGVVQPGRSLRLSCAASGFPFSNYVMYWVRQAPGKGLEWVALIFFDGKKNYHADSVKGRFTITRDNSKN
MLYLQMNSLRPEDAAVYYCAKIHCPNGVCYKGYYGMDVWGQGTTVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPKSCDK
TH;
or
(SEQ ID NO: 609)
QVQLVESGGGVVQPGRSLRLSCAASGFPFSNYVMYWVRQAPGKGLEWVALIFFDGKKNYHADSVKGRFTITRDNSKN
MLYLQMNSLRPEDAAVYYCAKIHCPNGVCYKGYYGMDVWGQGTTVTVSSASTKGPSVFPLAPCSRSTSESTAALGCL
VKDYFPEPVTVSWNSGALTSGVHTFPAVLQSSGLYSLSSVVTVPSSSLGTKTYTCNVDHKPSNTKVDKRVESKYGPP
LLQGSG;
or
(SEQ ID NO: 638)
QVQLVESGGGVVQPGRSLRLSCAASGFPFSNYVMYWVRQAPGKGLEWVALIFFDGKKNYHADSVKGRFTITRDNSKN
MLYLQMNSLRPEDAAVYYCAKIHCPNGVCYKGYYGMDVWGQGTTVTVSSASTKGPSVFPLAPCSRSTSESTAALGCL
VKDYFPEPVTVSWNSGALTSGVHTFPAVLQSSGLYSLSSVVTVPSSSLGTKTYTCNVDHKPSNTKVDKRVESKYGPP
(29) H1H12841B
Fab Light Chain
(SEQ ID NO: 596)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC
Fab Heavy Chain
(SEQ ID NO: 597)
EVQLVESGGGLVQPGGSLRLSCAASGFTFSNYWMNWVRQAPGKGLEWVANIKEDGGKKLYVDSVKGRFTISRDNAKN
SLFLQMNSLRAEDTAVYYCAREDTTLVVDYYYYGMDVWGQGTTVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCDKT
H
(30) 12850B
Fab Light Chain
(SEQ ID NO: 598)
EIVLTQSPGTLSLSPGERATLSCRASQSVSSSYLAWYQQKPGQAPRLLIYGASSRATGIPDRFSGSGSGTDFTLTIS
RLEPEDFAVYYCQQYGSSPWTFGQGTKVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC
Fab Heavy Chain
(SEQ ID NO: 599)
QVQLVQSGAEVKKPGSSVKVSCKASGGTFNTYAITWVRQAPGQGLEWMGGIIPISGIAEYAQKFQGRVTITTDDSST
TAYMELNSLRSEDTAVYYCASWNYALYYFYGMDVWGRGTTVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
(31) 69261
Fab Light Chain
(SEQ ID NO: 600)
DIVMTQSPLSLPVTPGEPASISCRSSQSLLHSNGYNYLDWYLQKPGQSPQFLIYLGSNRASGVPDRFSGSGSGTDFT
LKINRVEAEDVGVYYCMQALQTPYTFGQGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGEC
Fab Heavy Chain
(SEQ ID NO: 601)
QVQLVESGGGLVKPGGSLRLSCAASGFTFSVYYMNWIRQAPGKGLEWVSYISSSGSTIYYADSVKGRFTISRDNAKN
SLYLQMNSLRAEDTAVYYCGREGYSGTYSYYGMDVWGQGTTVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKTH
(32) 69263
Fab Light Chain
(SEQ ID NO: 602)
DIQMTQSPSSLSASVGDRVTITCRASQDISHYSAWYQQKPGKLPNLLIYAASTLQSGVPSRFSGSGSGTDFSLTTSS
LQPEDVATYYCQKYNSVPLTFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 603)
EVQLVESGGGLVQPGRSLRLSCAVSGFTFDDYAMHWVRQAPGKGLEWVSGISWNSGTRGYADSVKGRFTISRDNAKN
SLYLQMNSLRGEDTALYYCVKDITISPNYYGMDVWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH

In an embodiment, an anti-TfR antigen-binding protein, e.g., antibody or antigen-binding fragment (which may be tethered to a payload) comprises an IgG1 heavy chain constant domain comprising the sequence set forth in SEQ ID NO: 840: ASTKGPSVFPLAPSSKSTSGGTAALGCLVKDYFPEPVTVSWNSGALTSGVHTFPAVLQSSGLYSLSSVVTVPSSSLG TQTYICNVNHKPSNTKVDKKVEPKSCDKTHTCPPCPAPELLGGPSVFLFPPKPKDTLMISRTPEVTCVVVDVSHEDP EVKFNWYVDGVEVHNAKTKPREEQYNSTYRVVSVLTVLHQDWLNGKEYKCKVSNKALPAPIEKTISKAKGQPREPQV YTLPPSRDELTKNQVSLTCLVKGFYPSDIAVEWESNGQPENNYKTTPPVLDSDGSFFLYSKLTVDKSRWQQGNVFSC SVMHEALHNHYTQKSLSLSPGK (see, e.g., sequences of Table 4, or variants thereof). In an embodiment, an antigen-binding protein, e.g., antibody or antigen-binding fragment, comprises a light chain constant domain, e.g., of the type kappa or lambda. In an embodiment, a V_{H} as set forth herein is linked to a human heavy chain constant domain (e.g., IgG) and a V_{L} as set forth herein is linked to a human light chain constant domain (e.g., kappa). The present disclosure includes antigen-binding proteins comprising the variable domains set forth herein, which are linked to a heavy and/or light chain constant domain, e.g., as set forth herein.

TABLE 4
Heavy Chain Full hIgG1 Sequences.

| Identifier | HC Full hIgG1 sequence |
|---|---|
| 31874B | EVQLVESGGGLVQPGGSLRLSCAASGFAFSSYAMTWVRQAPGKGLEWVSVISGTGGSTYYADSVKG |
| | RFTISRDNSKNTLYLQMNSLRAEDTAVYYCAKGGAARRMEYFQYWGQGTLVTVSSASTKGPSVFPL |
| | APSSKSTSGGTAALGCLVKDYFPEPVTVSWNSGALTSGVHTFPAVLQSSGLYSLSSVVTVPSSSLG |
| | TQTYICNVNHKPSNTKVDKKVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK (SEQ ID |
| | NO: 808) |
| 31863B | EVQLVESGGGLVQPGGSLRLSCAASGFTFNSYAMTWVRQAPGKGLEWVSFIGGSTGNTYYAGSVKG |
| | RFTISSDNSKKTLYLQMNSLRAEDTAVYYCAKGGAARRMEYFQHWGQGTLVTVSSASTKGPSVFPL |
| | APSSKSTSGGTAALGCLVKDYFPEPVTVSWNSGALTSGVHTFPAVLQSSGLYSLSSVVTVPSSSLG |
| | TQTYICNVNHKPSNTKVDKKVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK (SEQ ID |
| | NO: 809) |
| 69348 | QVQLVESGGGVVQPGRSLRLSCAASGFTFTTYGMHWVRQAPGKGLEWVAVIWYDGSNKYYGDSVKG |
| | RFTISRDNSKNTLYLQMNSLRVDDTAVYYCTRTHGYTRSSDGFDYWGQGTLVTVSSASTKGPSVFP |
| | LAPSSKSTSGGTAALGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK (SEQ ID |
| | NO: 810) |
| 69340 | EVQLVESGGGLVQPGRSLRLSCAASGFTFDDKAMHWVRQVPGKGLEWISGISWNSGTIGYADSVKG |
| | RFIISRDNAKNSLYLQMNSLRAEDTALYYCAKDGDTSGWYWYGLDVWGQGTTVTVSSASTKGPSVF |
| | PLAPSSKSTSGGTAALGCLVKDYFPEPVTVSWNSGALTSGVHTFPAVLQSSGLYSLSSVVTVPSSS |
| | LGTQTYICNVNHKPSNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQKSLSLSPGK (SEQ ID |
| | NO: 811) |
| 69331 | QVQLVESGGGVVQPGRSLRLSCIASGFTFSVYGIHWVRQAPGKGLEWMAVISHDGNIKHYADSVKG |
| | RFTISRDNSKNTLYLQINSLRTEDTAVYYCAKDTWNSLDTFDIWGQGTMVTVSSASTKGPSVFPLA |
| | PSSKSTSGGTAALGCLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPKPKDTLMISRTPEVTC |
| | VVVDVSHEDPEVKFNWYVDGVEVHNAKTKPREEQYNSTYRVVSVLTVLHQDWLNGKEYKCKVSNKA |
| | LPAPIEKTISKAKGQPREPQVYTLPPSRDELTKNQVSLTCLVKGFYPSDIAVEWESNGQPENNYKT |
| | TPPVLDSDGSFFLYSKLTVDKSRWQQGNVFSCSVMHEALHNHYTQKSLSLSPGK (SEQ ID NO: |
| | 812) |
| 69332 | QVTLRESGPALVKPSQTLTLTCTFSGFSLNTYGMFVSWIRQPPGKALEWLAHIHWDDDKYYSTSLK |
| | TRLTISKDTSKNQVVLTMTNMDPVDTATYYCARGHNNLNYIIHWGQGTLVTVSSASTKGPSVFPLA |
| | PSSKSTSGGTAALGCLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPKPKDTLMISRTPEVTC |
| | VVVDVSHEDPEVKFNWYVDGVEVHNAKTKPREEQYNSTYRVVSVLTVLHQDWLNGKEYKCKVSNKA |
| | LPAPIEKTISKAKGQPREPQVYTLPPSRDELTKNQVSLTCLVKGFYPSDIAVEWESNGQPENNYKT |
| | TPPVLDSDGSFFLYSKLTVDKSRWQQGNVFSCSVMHEALHNHYTQKSLSLSPGK (SEQ ID NO: |
| | 813) |
| 69326 | EVQLVESGGGLVQPGGSLRLSCAVSGFIFSSYEMNWVRQAPGKGLEWVSYISSSGSTIFYADSVKG |
| | RFTISRDNAKNSLYLQMNSLRAEDTAVYYCVSGVVLFDVWGQGTMVTVSSASTKGPSVFPLAPSSK |
| | STSGGTAALGCLVKDYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQDWLNGKEYKCKVSNKALPAP |
| | IEKTISKAKGQPREPQVYTLPPSRDELTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK (SEQ ID NO: |
| | 814) |
| 69329 | EVQLVESGGGLVQPGGSLRLSCAASGFTFSNYWMTWVRQAPGKGLEWVANIKEDGSEKDYVDSVKG |
| | RFTISRDNAKNSLYLQMNSLRGEDTAVYYCARDGEQLVDYYYYYVMDVWGQGTTVTVSSASTKGPS |
| | VFPLAPSSKSTSGGTAALGCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSLSPGK (SEQ |
| | ID NO: 815) |
| 69323 | EVQLVESGGGLVQPGRSLRLSCAASGFTFDDYAMHWVRQAPGKGLEWVSGISWNSGYIGYADSVKG |
| | RFTISRDNAENSLHLQMNSLRAEDTALYYCARGGSTLVRGVKGGYYGMDVWGQGTTVTVSSASTKG |
| | PSVFPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVVSVLTVLHQDWLNGKEYK |
| | CKVSNKALPAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGK (SEQ |
| | ID NO: 816) |
| 69305 | QVQLVESGGGVVQPGRSLRLSCAASGFTFSSYGMHWVRQAPGKGLEWVAVIWYDGSNKYYADSVKG |
| | RFTISRDISKNTLYLQMNSLRAEDTAVYYCAGQLDLFFDYWGQGTLVTVSSASTKGPSVFPLAPSS |
| | KSTSGGTAALGCLVKDYFPEPVTVSWNSGALTSGVHTFPAVLQSSGLYSLSSVVTVPSSSLGTQTY |
| | ICNVNHKPSNTKVDKKVEPKSCDKTHTCPPCPAPELLGGPSVFLFPPKPKDTLMISRTPEVTCVVV |
| | DVSHEDPEVKFNWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNGQPENNYKTTPP |
| | VLDSDGSFFLYSKLTVDKSRWQQGNVFSCSVMHEALHNHYTQKSLSLSPGK (SEQ ID NO: |
| | 817) |
| 69307 | EVQLVESGGGLVQPGGSLRLSCTASGFTFSNYWMTWVRQAPGKGLEWVANIKEDGSEKEYVDSVKG |
| | RFTISRDNAKNSLYLQMNSLRGEDTAVYYCARDGEQLVDYYYYYVMDVWGQGTTVTVSSASTKGPS |
| | VFPLAPSSKSTSGGTAALGCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSLSPGK (SEQ |
| | ID NO: 818) |
| 12795B | EVQLVESGGGLVQPGGSLRLSCATSGFTFTSYDMKWVRQAPGLGLEWVSAISGSGGNTYYADSVKG |
| | RFTISRDNSRNTLYLQMNSLRAEDTAVYYCTRSHDFGAFDYFDYWGQGTLVTVSSASTKGPSVFPL |
| | APSSKSTSGGTAALGCLVKDYFPEPVTVSWNSGALTSGVHTFPAVLQSSGLYSLSSVVTVPSSSLG |
| | TQTYICNVNHKPSNTKVDKKVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK (SEQ ID |
| | NO: 819) |
| 12798B | EVQLVESGGDLVQPGRSLRLSCAASGFTFDDYAMHWVRQAPGKGLEWVSGISWNSATRVYADSVKG |
| | RFTISRDNAKNFLYLQMNSLRSEDTALYHCAKDMDISLGYYGLDVWGQGTTVTVSSASTKGPSVFP |
| | LAPSSKSTSGGTAALGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK (SEQ ID |
| | NO: 820) |
| 12799B | QITLKESGPTLVKPTQTLTLTCTFSGFSLSTSGVGVVWIRQPPGKALEWLALIYWNDHKRYSPSLG |
| | SRLTITKDTSKNQVVLTMTNMDPVDTATYYCAHYSGSYSYYYYGLDVWGQGTTVTVSSASTKGPSV |
| | FPLAPSSKSTSGGTAALGCLVKDYFPEPVTVSWNSGALTSGVHTFPAVLQSSGLYSLSSVVTVPSS |
| | SLGTQTYICNVNHKPSNTKVDKKVEPKSCDKTHTCPPCPAPELLGGPSVFLFPPKPKDTLMISRTP |
| | EVTCVVVDVSHEDPEVKFNWYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQKSLSLSPGK (SEQ ID |
| | NO: 821) |
| 12801B | EVQLLESGGALVQPGGSLRLSCAASGFTFTSYAMHWVRQAPGKGLEWVSSIRGSGGGTYSADSVKG |
| | RFTISRDNSRDTLYLQMNSVRAEDTAVYYCARSHDYGAFDFFDYWGQGTLVTVSSASTKGPSVFPL |
| | APSSKSTSGGTAALGCLVKDYFPEPVTVSWNSGALTSGVHTFPAVLQSSGLYSLSSVVTVPSSSLG |
| | TQTYICNVNHKPSNTKVDKKVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK (SEQ ID |
| | NO: 822) |
| 12802B | QVQLVESGGGLVKPGGSLRLSCAASGFTFSDYFMSWIRQAPGKGLEWVSYISSTGSTINYADSVKG |
| | RFTISRDNVKNSLYLQMTSLRVEDTAVYYCTRDNWNYEYWGQGTLVTVSSASTKGPSVFPLAPSSK |
| | STSGGTAALGCLVKDYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQDWLNGKEYKCKVSNKALPAP |
| | IEKTISKAKGQPREPQVYTLPPSRDELTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK (SEQ ID NO: |
| | 823) |
| 12808B | QLQLQESGPGLVKPSETLSLTCTVSGESISSNTYYWGWIRQPPGKGLEWIGSIDYSGTTNYNPSLK |
| | SRVTISVDTSRNHFSLRLRSVTAADTAVYYCAREWGNYGYYYGMDVWGQGTTVTVSSASTKGPSVF |
| | PLAPSSKSTSGGTAALGCLVKDYFPEPVTVSWNSGALTSGVHTFPAVLQSSGLYSLSSVVTVPSSS |
| | LGTQTYICNVNHKPSNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQKSLSLSPGK (SEQ ID |
| | NO: 824) |
| 12812B | QVQLVQSGAEVKKPGSSVRVSCKASRGTFSSYAISWVRQAPGQGLEWMGGIIPIFGTANYAQKFLA |
| | RVTITADESTSTAYMELSSLRSEDTAVYYCAREKGWNYFDYWGQGTLVTVSSASTKGPSVFPLAPS |
| | SKSTSGGTAALGCLVKDYFPEPVTVSWNSGALTSGVHTFPAVLQSSGLYSLSSVVTVPSSSLGTQT |
| | YICNVNHKPSNTKVDKKVEPKSCDKTHTCPPCPAPELLGGPSVFLFPPKPKDTLMISRTPEVTCVV |
| | VDVSHEDPEVKFNWYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQPENNYKTTP |
| | PVLDSDGSFFLYSKLTVDKSRWQQGNVFSCSVMHEALHNHYTQKSLSLSPGK (SEQ ID NO: |
| | 825) |
| 12816B | QVQLVESGGGLVKPGGSLRLSCAASGFTFSDYYMNWIRQAPGKGLEWVSYISSSGTTIYYADSVKG |
| | RFTISRDNAKKSLYLEMNSLRAEDTAVYYCAREGYGNDYYYYGIDVWGQGTTVTVSSASTKGPSVF |
| | PLAPSSKSTSGGTAALGCLVKDYFPEPVTVSWNSGALTSGVHTFPAVLQSSGLYSLSSVVTVPSSS |
| | LGTQTYICNVNHKPSNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQKSLSLSPGK (SEQ ID |
| | NO: 826) |
| 12833B | QVQLVESGGGVVQPGRSLRLSCAASGFTFSSFGMHWVRQAPGKGLEWVIFISYDGSDKYYADSVKG |
| | RFAISRDSSKNTLYLQMNSLRAEDTAVYYCAKENGILTDSYGMDVWGQGTTVTVSSASTKGPSVFP |
| | LAPSSKSTSGGTAALGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPPKPKDTLMISRTPEV |
| | TCVVVDVSHEDPEVKENWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK (SEQ ID |
| | NO: 827) |
| 12834B | QVQLVQSGAEVKKPGASVKVSCKASGYTFTSYGISWVRQAPGQGLEWMGWISVYHGNTNYAQKFQG |
| | RVTMTTDTSTSTAYMELRSLRSDDTAVYYCAREGYYDFWSGYYPFDYWGQGTLVTVSSASTKGPSV |
| | FPLAPSSKSTSGGTAALGCLVKDYFPEPVTVSWNSGALTSGVHTFPAVLQSSGLYSLSSVVTVPSS |
| | SLGTQTYICNVNHKPSNTKVDKKVEPKSCDKTHTCPPCPAPELLGGPSVFLFPPKPKDTLMISRTP |
| | EVTCVVVDVSHEDPEVKFNWYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQKSLSLSPGK (SEQ ID |
| | NO: 828) |
| 12835B | EVQLVESGGGLIQPGGSLRLSCEASGFTFRNYEMNWVRQAPGKGLEWVSYISSSGNMKDYAESVKG |
| | RFTISRDNVKNSLQLQMNSLRVEDTAVYYCARDEFPYGMDVWGQGTTVTVSSASTKGPSVFPLAPS |
| | SKSTSGGTAALGCLVKDYFPEPVTVSWNSGALTSGVHTFPAVLQSSGLYSLSSVVTVPSSSLGTQT |
| | YICNVNHKPSNTKVDKKVEPKSCDKTHTCPPCPAPELLGGPSVFLFPPKPKDTLMISRTPEVTCVV |
| | VDVSHEDPEVKFNWYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQPENNYKTTP |
| | PVLDSDGSFFLYSKLTVDKSRWQQGNVFSCSVMHEALHNHYTQKSLSLSPGK (SEQ ID NO: |
| | 829) |
| 12847B | EVQLVESGGGLVQPGRSLRLSCAASGFTFDDYAMNWVRQAPGKGLEWVSGISWSSGSMDYADSVKG |
| | RFTISRDNAKNSLYLQMNSLRTEDTALYYCAKAREVGDYYGMDVWGQGTTVTVSSASTKGPSVFPL |
| | APSSKSTSGGTAALGCLVKDYFPEPVTVSWNSGALTSGVHTFPAVLQSSGLYSLSSVVTVPSSSLG |
| | TQTYICNVNHKPSNTKVDKKVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK (SEQ ID |
| | NO: 830) |
| 12848B | EVQLVESGGGLVQPGRSLTLSCAASGFTFDNFGMHWVRQGPGKGLEWVSGLTWNSGVIGYADSVKG |
| | RFTISRDNAKNSLYLQMNSLRPEDTALYYCAKDIRNYGPFDYWGQGTLVTVSSASTKGPSVFPLAP |
| | SSKSTSGGTAALGCLVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVSNKAL |
| | PAPIEKTISKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGK (SEQ ID NO: |
| | 831) |
| 12843B | EVQLVESGGGLVQPGGSLRLSCAASGFTFNIFEMNWVRQAPGKGLEWISYISSRGTTTYYADSVRG |
| | RFTISRDNAKNSLYLQMNSLRAEDTAVYYCARDYEATIPFDFWGQGTLVTVSSASTKGPSVFPLAP |
| | SSKSTSGGTAALGCLVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVSNKAL |
| | PAPIEKTISKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGK (SEQ ID NO: |
| | 832) |
| 12844B | EVQLVESGGSVVRPGGSLRLSCEASGFTFDDYGMSWVRQDPGKGLEWVSGINWNGDRTNYADSVKG |
| | RFIISRDNAKNSVYLQMNSLRAEDSALYHCARDQGLGVAATLDYWGQGTLVTVSSASTKGPSVFPL |
| | APSSKSTSGGTAALGCLVKDYFPEPVTVSWNSGALTSGVHTFPAVLQSSGLYSLSSVVTVPSSSLG |
| | TQTYICNVNHKPSNTKVDKKVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK (SEQ ID |
| | NO: 833) |
| 12845B | EVQLVESGGGLVQPGGSLRLSCAASGFTVSNYEMNWVRQAPGKGLEWVSYISSSTSNIYYADSVKG |
| | RFTISRDNAENSLYLQMNSLRVEDTAVYYCVRDGIVVVPVGRGYYYYGLDVWGQGTTVTVSSASTK |
| | GPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRVVSVLTVLHQDWLNGKEY |
| | KCKVSNKALPAPIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKSLSLSPGK |
| | (SEQ ID NO: 834) |
| 12839B | QVQLVESGGGVVQPGRSLRLSCAASGFPFSNYVMYWVRQAPGKGLEWVALIFFDGKKNYHADSVKG |
| | RFTITRDNSKNMLYLQMNSLRPEDAAVYYCAKIHCPNGVCYKGYYGMDVWGQGTTVTVSSASTKGP |
| | SVFPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVSVLTVLHQDWLNGKEYKC |
| | KVSNKALPAPIEKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLSLSPGK (SEQ |
| | ID NO: 835) |
| 12841B | EVQLVESGGGLVQPGGSLRLSCAASGFTFSNYWMNWVRQAPGKGLEWVANIKEDGGKKLYVDSVKG |
| | RFTISRDNAKNSLFLQMNSLRAEDTAVYYCAREDTTLVVDYYYYGMDVWGQGTTVTVSSASTKGPS |
| | VFPLAPSSKSTSGGTAALGCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSLSPGK (SEQ |
| | ID NO: 836) |
| 12850B | QVQLVQSGAEVKKPGSSVKVSCKASGGTFNTYAITWVRQAPGQGLEWMGGIIPISGIAEYAQKFQG |
| | RVTITTDDSSTTAYMELNSLRSEDTAVYYCASWNYALYYFYGMDVWGRGTTVTVSSASTKGPSVFP |
| | LAPSSKSTSGGTAALGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK (SEQ ID |
| | NO: 837) |
| 69261 | QVQLVESGGGLVKPGGSLRLSCAASGFTFSVYYMNWIRQAPGKGLEWVSYISSSGSTIYYADSVKG |
| | RFTISRDNAKNSLYLQMNSLRAEDTAVYYCGREGYSGTYSYYGMDVWGQGTTVTVSSASTKGPSVF |
| | PLAPSSKSTSGGTAALGCLVKDYFPEPVTVSWNSGALTSGVHTFPAVLQSSGLYSLSSVVTVPSSS |
| | LGTQTYICNVNHKPSNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQKSLSLSPGK (SEQ ID |
| | NO: 838) |
| 69263 | EVQLVESGGGLVQPGRSLRLSCAVSGFTFDDYAMHWVRQAPGKGLEWVSGISWNSGTRGYADSVKG |
| | RFTISRDNAKNSLYLQMNSLRGEDTALYYCVKDITISPNYYGMDVWGQGTTVTVSSASTKGPSVFP |
| | LAPSSKSTSGGTAALGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK (SEQ ID |
| | NO: 839) |

As discussed, an anti-hTfR:GAA scFv fusion protein (e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263) comprises an optional signal peptide, connected to an scFv (e.g., including a V_{L} and a V_{H} optionally connected by a linker), connected to an option linker, connected to a GAA polypeptide. For example, the optional signal peptide can be the signal peptide from Mus musculus Ror1 (e.g., consisting of the amino acids M4HRPRRRGTRPPPLALLAALLLAARGADA (SEQ ID NO: 610).

In a particular multidomain therapeutic protein, the TfR-binding delivery domain is an anti-TfR scFv. For example, the scFv can include a V_{L} and a V_{H} optionally connected by a linker.

In one example, the anti-hTfR antibody or antigen-binding fragment thereof or scFv can comprise: (i) a heavy chain variable region that comprises the HCDR1, HCDR2 and HCDR3 of a HCVR comprising the amino acid sequence set forth in SEQ ID NO: 171, 680, 181, 681, 191, 682, 201, 211, 221, 685, 231, 687, 241, 689, 251, 261, 691, 271, 281, 692, 291, 301, 311, 694, 321, 331, 696, 341, 351, 697, 361, 699, 371, 700, 381, 391, 401, 411, 421, 701, 431, 441, 451, 461, 471, 702, or 481; and/or (ii) a light chain variable region that comprises the LCDR1, LCDR2 and LCDR3 of a LCVR comprising the amino acid sequence set forth in SEQ ID NO: 176, 186, 196, 206, 683, 216, 684, 226, 686, 236, 688, 246, 690, 256, 266, 276, 286, 693, 296, 306, 316, 695, 326, 336, 346, 356, 698, 366, 376, 386, 396, 406, 416, 426, 436, 446, 456, 466, 476, 632, 486, or 703.

In another example, the anti-TfR antibody or antigen-binding fragment thereof or scFv can comprise: (1) a HCVR comprising the HCDR1, HCDR2 and HCDR3 of a HCVR that comprises the amino acid sequence set forth in SEQ ID NO: 171 or 680 (or a variant thereof); and a LCVR comprising the LCDR1, LCDR2 and LCDR3 of a LCVR that comprises the amino acid sequence set forth in SEQ ID NO: 176 (or a variant thereof); (2) a HCVR comprising the HCDR1, HCDR2 and HCDR3 of a HCVR that comprises the amino acid sequence set forth in SEQ ID NO: 181 or 681 (or a variant thereof); and a LCVR comprising the LCDR1, LCDR2 and LCDR3 of a LCVR that comprises the amino acid sequence set forth in SEQ ID NO: 186 (or a variant thereof); (3) a HCVR comprising the HCDR1, HCDR2 and HCDR3 of a HCVR that comprises the amino acid sequence set forth in SEQ ID NO: 191 or 682 (or a variant thereof); and a LCVR comprising the LCDR1, LCDR2 and LCDR3 of a LCVR that comprises the amino acid sequence set forth in SEQ ID NO: 196 (or a variant thereof); (4) a HCVR comprising the HCDR1, HCDR2 and HCDR3 of a HCVR that comprises the amino acid sequence set forth in SEQ ID NO: 201 (or a variant thereof); and a LCVR comprising the LCDR1, LCDR2 and LCDR3 of a LCVR that comprises the amino acid sequence set forth in SEQ ID NO: 206 or 683 (or a variant thereof); (5) a HCVR comprising the HCDR1, HCDR2 and HCDR3 of a HCVR that comprises the amino acid sequence set forth in SEQ ID NO: 211 (or a variant thereof); and a LCVR comprising the LCDR1, LCDR2 and LCDR3 of a LCVR that comprises the amino acid sequence set forth in SEQ ID NO: 216 or 684 (or a variant thereof); (6) a HCVR comprising the HCDR1, HCDR2 and HCDR3 of a HCVR that comprises the amino acid sequence set forth in SEQ ID NO: 221 or 685 (or a variant thereof); and a LCVR comprising the LCDR1, LCDR2 and LCDR3 of a LCVR that comprises the amino acid sequence set forth in SEQ ID NO: 226 or 686 (or a variant thereof); (7) a HCVR comprising the HCDR1, HCDR2 and HCDR3 of a HCVR that comprises the amino acid sequence set forth in SEQ ID NO: 231 or 687 (or a variant thereof); and a LCVR comprising the LCDR1, LCDR2 and LCDR3 of a LCVR that comprises the amino acid sequence set forth in SEQ ID NO: 236 or 688 (or a variant thereof); (8) a HCVR comprising the HCDR1, HCDR2 and HCDR3 of a HCVR that comprises the amino acid sequence set forth in SEQ ID NO: 241 or 689 (or a variant thereof); and a LCVR comprising the LCDR1, LCDR2 and LCDR3 of a LCVR that comprises the amino acid sequence set forth in SEQ ID NO: 246 or 690 (or a variant thereof); (9) a HCVR comprising the HCDR1, HCDR2 and HCDR3 of a HCVR that comprises the amino acid sequence set forth in SEQ ID NO: 251 (or a variant thereof); and a LCVR comprising the LCDR1, LCDR2 and LCDR3 of a LCVR that comprises the amino acid sequence set forth in SEQ ID NO: 256 (or a variant thereof); (10) a HCVR comprising the HCDR1, HCDR2 and HCDR3 of a HCVR that comprises the amino acid sequence set forth in SEQ ID NO: 261 or 691 (or a variant thereof); and a LCVR comprising the LCDR1, LCDR2 and LCDR3 of a LCVR that comprises the amino acid sequence set forth in SEQ ID NO: 266 (or a variant thereof); (11) a HCVR comprising the HCDR1, HCDR2 and HCDR3 of a HCVR that comprises the amino acid sequence set forth in SEQ ID NO: 271 (or a variant thereof); and a LCVR comprising the LCDR1, LCDR2 and LCDR3 of a LCVR that comprises the amino acid sequence set forth in SEQ ID NO: 276 (or a variant thereof); (12) a HCVR comprising the HCDR1, HCDR2 and HCDR3 of a HCVR that comprises the amino acid sequence set forth in SEQ ID NO: 281 or 692 (or a variant thereof); and a LCVR comprising the LCDR1, LCDR2 and LCDR3 of a LCVR that comprises the amino acid sequence set forth in SEQ ID NO: 286 or 693 (or a variant thereof); (13) a HCVR comprising the HCDR1, HCDR2 and HCDR3 of a HCVR that comprises the amino acid sequence set forth in SEQ ID NO: 291 (or a variant thereof); and a LCVR comprising the LCDR1, LCDR2 and LCDR3 of a LCVR that comprises the amino acid sequence set forth in SEQ ID NO: 296 (or a variant thereof); (14) a HCVR comprising the HCDR1, HCDR2 and HCDR3 of a HCVR that comprises the amino acid sequence set forth in SEQ ID NO: 301 (or a variant thereof); and a LCVR comprising the LCDR1, LCDR2 and LCDR3 of a LCVR that comprises the amino acid sequence set forth in SEQ ID NO: 306 (or a variant thereof); (15) a HCVR comprising the HCDR1, HCDR2 and HCDR3 of a HCVR that comprises the amino acid sequence set forth in SEQ ID NO: 311 or 694 (or a variant thereof); and a LCVR comprising the LCDR1, LCDR2 and LCDR3 of a LCVR that comprises the amino acid sequence set forth in SEQ ID NO: 316 or 695 (or a variant thereof); (16) a HCVR comprising the HCDR1, HCDR2 and HCDR3 of a HCVR that comprises the amino acid sequence set forth in SEQ ID NO: 321 (or a variant thereof); and a LCVR comprising the LCDR1, LCDR2 and LCDR3 of a LCVR that comprises the amino acid sequence set forth in SEQ ID NO: 326 (or a variant thereof); (17) a HCVR comprising the HCDR1, HCDR2 and HCDR3 of a HCVR that comprises the amino acid sequence set forth in SEQ ID NO: 331 or 696 (or a variant thereof); and a LCVR comprising the LCDR1, LCDR2 and LCDR3 of a LCVR that comprises the amino acid sequence set forth in SEQ ID NO: 336 (or a variant thereof); (18) a HCVR comprising the HCDR1, HCDR2 and HCDR3 of a HCVR that comprises the amino acid sequence set forth in SEQ ID NO: 341 (or a variant thereof); and a LCVR comprising the LCDR1, LCDR2 and LCDR3 of a LCVR that comprises the amino acid sequence set forth in SEQ ID NO: 346 (or a variant thereof); (19) a HCVR comprising the HCDR1, HCDR2 and HCDR3 of a HCVR that comprises the amino acid sequence set forth in SEQ ID NO: 351 or 697 (or a variant thereof); and a LCVR comprising the LCDR1, LCDR2 and LCDR3 of a LCVR that comprises the amino acid sequence set forth in SEQ ID NO: 356 or 698 (or a variant thereof); (20) a HCVR comprising the HCDR1, HCDR2 and HCDR3 of a HCVR that comprises the amino acid sequence set forth in SEQ ID NO: 361 or 699 (or a variant thereof); and a LCVR comprising the LCDR1, LCDR2 and LCDR3 of a LCVR that comprises the amino acid sequence set forth in SEQ ID NO: 366 (or a variant thereof); (21) a HCVR comprising the HCDR1, HCDR2 and HCDR3 of a HCVR that comprises the amino acid sequence set forth in SEQ ID NO: 371 or 700 (or a variant thereof); and a LCVR comprising the LCDR1, LCDR2 and LCDR3 of a LCVR that comprises the amino acid sequence set forth in SEQ ID NO: 376 (or a variant thereof); (22) a HCVR comprising the HCDR1, HCDR2 and HCDR3 of a HCVR that comprises the amino acid sequence set forth in SEQ ID NO: 381 (or a variant thereof); and a LCVR comprising the LCDR1, LCDR2 and LCDR3 of a LCVR that comprises the amino acid sequence set forth in SEQ ID NO: 386 (or a variant thereof); (23) a HCVR comprising the HCDR1, HCDR2 and HCDR3 of a HCVR that comprises the amino acid sequence set forth in SEQ ID NO: 391 (or a variant thereof); and a LCVR comprising the LCDR1, LCDR2 and LCDR3 of a LCVR that comprises the amino acid sequence set forth in SEQ ID NO: 396 (or a variant thereof); (24) a HCVR comprising the HCDR1, HCDR2 and HCDR3 of a HCVR that comprises the amino acid sequence set forth in SEQ ID NO: 401 (or a variant thereof); and a LCVR comprising the LCDR1, LCDR2 and LCDR3 of a LCVR that comprises the amino acid sequence set forth in SEQ ID NO: 406 (or a variant thereof); (25) a HCVR comprising the HCDR1, HCDR2 and HCDR3 of a HCVR that comprises the amino acid sequence set forth in SEQ ID NO: 411 (or a variant thereof); and a LCVR comprising the LCDR1, LCDR2 and LCDR3 of a LCVR that comprises the amino acid sequence set forth in SEQ ID NO: 416 (or a variant thereof); (26) a HCVR comprising the HCDR1, HCDR2 and HCDR3 of a HCVR that comprises the amino acid sequence set forth in SEQ ID NO: 421 or 701 (or a variant thereof); and a LCVR comprising the LCDR1, LCDR2 and LCDR3 of a LCVR that comprises the amino acid sequence set forth in SEQ ID NO: 426 (or a variant thereof); (27) a HCVR comprising the HCDR1, HCDR2 and HCDR3 of a HCVR that comprises the amino acid sequence set forth in SEQ ID NO: 431 (or a variant thereof); and a LCVR comprising the LCDR1, LCDR2 and LCDR3 of a LCVR that comprises the amino acid sequence set forth in SEQ ID NO: 436 (or a variant thereof); (28) a HCVR comprising the HCDR1, HCDR2 and HCDR3 of a HCVR that comprises the amino acid sequence set forth in SEQ ID NO: 441 (or a variant thereof); and a LCVR comprising the LCDR1, LCDR2 and LCDR3 of a LCVR that comprises the amino acid sequence set forth in SEQ ID NO: 446 (or a variant thereof); (29) a HCVR comprising the HCDR1, HCDR2 and HCDR3 of a HCVR that comprises the amino acid sequence set forth in SEQ ID NO: 451 (or a variant thereof); and a LCVR comprising the LCDR1, LCDR2 and LCDR3 of a LCVR that comprises the amino acid sequence set forth in SEQ ID NO: 456 (or a variant thereof); (30) a HCVR comprising the HCDR1, HCDR2 and HCDR3 of a HCVR that comprises the amino acid sequence set forth in SEQ ID NO: 461 (or a variant thereof); and a LCVR comprising the LCDR1, LCDR2 and LCDR3 of a LCVR that comprises the amino acid sequence set forth in SEQ ID NO: 466 (or a variant thereof); (31) a HCVR comprising the HCDR1, HCDR2 and HCDR3 of a HCVR that comprises the amino acid sequence set forth in SEQ ID NO: 471 or 702 (or a variant thereof); and a LCVR comprising the LCDR1, LCDR2 and LCDR3 of a LCVR that comprises the amino acid sequence set forth in SEQ ID NO: 476 or 632 (or a variant thereof); or (32) a HCVR comprising the HCDR1, HCDR2 and HCDR3 of a HCVR that comprises the amino acid sequence set forth in SEQ ID NO: 481 (or a variant thereof); and a LCVR comprising the LCDR1, LCDR2 and LCDR3 of a LCVR that comprises the amino acid sequence set forth in SEQ ID NO: 486 or 703 (or a variant thereof). A variant refers to a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein.

In another example, the anti-TfR antibody or antigen-binding fragment thereof or scFv can comprise: (23) a HCVR comprising the HCDR1, HCDR2 and HCDR3 of a HCVR that comprises the amino acid sequence set forth in SEQ ID NO: 391 (or a variant thereof); and a LCVR comprising the LCDR1, LCDR2 and LCDR3 of a LCVR that comprises the amino acid sequence set forth in SEQ ID NO: 396 (or a variant thereof); or (25) a HCVR comprising the HCDR1, HCDR2 and HCDR3 of a HCVR that comprises the amino acid sequence set forth in SEQ ID NO: 411 (or a variant thereof); and a LCVR comprising the LCDR1, LCDR2 and LCDR3 of a LCVR that comprises the amino acid sequence set forth in SEQ ID NO: 416 (or a variant thereof). In another example, the anti-TfR antibody or antigen-binding fragment thereof or scFv can comprise a HCVR comprising the HCDR1, HCDR2 and HCDR3 of a HCVR that comprises the amino acid sequence set forth in SEQ ID NO: 391 (or a variant thereof); and a LCVR comprising the LCDR1, LCDR2 and LCDR3 of a LCVR that comprises the amino acid sequence set forth in SEQ ID NO: 396 (or a variant thereof). In another example, the anti-TfR antibody or antigen-binding fragment thereof or scFv can comprise a HCVR comprising the HCDR1, HCDR2 and HCDR3 of a HCVR that comprises the amino acid sequence set forth in SEQ ID NO: 411 (or a variant thereof); and a LCVR comprising the LCDR1, LCDR2 and LCDR3 of a LCVR that comprises the amino acid sequence set forth in SEQ ID NO: 416 (or a variant thereof).

In another example, the anti-TfR antibody or antigen-binding fragment thereof or scFv can comprise: (a) a HCVR that comprises: an HCDR1 comprising the amino acid sequence set forth in SEQ ID NO: 172 (or a variant thereof), an HCDR2 comprising the amino acid sequence set forth in SEQ ID NO: 173 (or a variant thereof), and an HCDR3 comprising the amino acid sequence set forth in SEQ ID NO: 174 (or a variant thereof); and a LCVR that comprises: an LCDR1 comprising the amino acid sequence set forth in SEQ ID NO: 177 (or a variant thereof), an LCDR2 comprising the amino acid sequence set forth in SEQ ID NO: 178 (or a variant thereof), and an LCDR3 comprising the amino acid sequence set forth in SEQ ID NO: 179 (or a variant thereof); (b) a HCVR that comprises: an HCDR1 comprising the amino acid sequence set forth in SEQ ID NO: 182 (or a variant thereof), an HCDR2 comprising the amino acid sequence set forth in SEQ ID NO: 183 (or a variant thereof), and an HCDR3 comprising the amino acid sequence set forth in SEQ ID NO: 184 (or a variant thereof); and a LCVR that comprises: an LCDR1 comprising the amino acid sequence set forth in SEQ ID NO: 187 (or a variant thereof), an LCDR2 comprising the amino acid sequence set forth in SEQ ID NO: 188 (or a variant thereof), and an LCDR3 comprising the amino acid sequence set forth in SEQ ID NO: 189 (or a variant thereof); (c) a HCVR that comprises: an HCDR1 comprising the amino acid sequence set forth in SEQ ID NO: 192 (or a variant thereof), an HCDR2 comprising the amino acid sequence set forth in SEQ ID NO: 193 (or a variant thereof), and an HCDR3 comprising the amino acid sequence set forth in SEQ ID NO: 194 (or a variant thereof); and a LCVR that comprises: an LCDR1 comprising the amino acid sequence set forth in SEQ ID NO: 197 (or a variant thereof), an LCDR2 comprising the amino acid sequence set forth in SEQ ID NO: 198 (or a variant thereof), and an LCDR3 comprising the amino acid sequence set forth in SEQ ID NO: 199 (or a variant thereof); (d) a HCVR that comprises: an HCDR1 comprising the amino acid sequence set forth in SEQ ID NO: 202 (or a variant thereof), an HCDR2 comprising the amino acid sequence set forth in SEQ ID NO: 203 (or a variant thereof), and an HCDR3 comprising the amino acid sequence set forth in SEQ ID NO: 204 (or a variant thereof); and a LCVR that comprises: an LCDR1 comprising the amino acid sequence set forth in SEQ ID NO: 207 (or a variant thereof), an LCDR2 comprising the amino acid sequence set forth in SEQ ID NO: 208 (or a variant thereof), and an LCDR3 comprising the amino acid sequence set forth in SEQ ID NO: 209 (or a variant thereof); (e) a HCVR that comprises: an HCDR1 comprising the amino acid sequence set forth in SEQ ID NO: 212 (or a variant thereof), an HCDR2 comprising the amino acid sequence set forth in SEQ ID NO: 213 (or a variant thereof), and an HCDR3 comprising the amino acid sequence set forth in SEQ ID NO: 214 (or a variant thereof); and a LCVR that comprises: an LCDR1 comprising the amino acid sequence set forth in SEQ ID NO: 217 (or a variant thereof), an LCDR2 comprising the amino acid sequence set forth in SEQ ID NO: 218 (or a variant thereof), and an LCDR3 comprising the amino acid sequence set forth in SEQ ID NO: 219 (or a variant thereof); (f) a HCVR that comprises: an HCDR1 comprising the amino acid sequence set forth in SEQ ID NO: 222 (or a variant thereof), an HCDR2 comprising the amino acid sequence set forth in SEQ ID NO: 223 (or a variant thereof), and an HCDR3 comprising the amino acid sequence set forth in SEQ ID NO: 224 (or a variant thereof); and a LCVR that comprises: an LCDR1 comprising the amino acid sequence set forth in SEQ ID NO: 227 (or a variant thereof), an LCDR2 comprising the amino acid sequence set forth in SEQ ID NO: 228 (or a variant thereof), and an LCDR3 comprising the amino acid sequence set forth in SEQ ID NO: 229 (or a variant thereof); (g) a HCVR that comprises: an HCDR1 comprising the amino acid sequence set forth in SEQ ID NO: 232 (or a variant thereof), an HCDR2 comprising the amino acid sequence set forth in SEQ ID NO: 233 (or a variant thereof), and an HCDR3 comprising the amino acid sequence set forth in SEQ ID NO: 234 (or a variant thereof); and a LCVR that comprises: an LCDR1 comprising the amino acid sequence set forth in SEQ ID NO: 237 (or a variant thereof), an LCDR2 comprising the amino acid sequence set forth in SEQ ID NO: 238 (or a variant thereof), and an LCDR3 comprising the amino acid sequence set forth in SEQ ID NO: 239 (or a variant thereof); (h) a HCVR that comprises: an HCDR1 comprising the amino acid sequence set forth in SEQ ID NO: 242 (or a variant thereof), an HCDR2 comprising the amino acid sequence set forth in SEQ ID NO: 243 (or a variant thereof), and an HCDR3 comprising the amino acid sequence set forth in SEQ ID NO: 244 (or a variant thereof); and a LCVR that comprises: an LCDR1 comprising the amino acid sequence set forth in SEQ ID NO: 247 (or a variant thereof), an LCDR2 comprising the amino acid sequence set forth in SEQ ID NO: 248 (or a variant thereof), and an LCDR3 comprising the amino acid sequence set forth in SEQ ID NO: 249 (or a variant thereof); (i) a HCVR that comprises: an HCDR1 comprising the amino acid sequence set forth in SEQ ID NO: 252 (or a variant thereof), an HCDR2 comprising the amino acid sequence set forth in SEQ ID NO: 253 (or a variant thereof), and an HCDR3 comprising the amino acid sequence set forth in SEQ ID NO: 254 (or a variant thereof); and a LCVR that comprises: an LCDR1 comprising the amino acid sequence set forth in SEQ ID NO: 257 (or a variant thereof), an LCDR2 comprising the amino acid sequence set forth in SEQ ID NO: 258 (or a variant thereof), and an LCDR3 comprising the amino acid sequence set forth in SEQ ID NO: 259 (or a variant thereof); (j) a HCVR that comprises: an HCDR1 comprising the amino acid sequence set forth in SEQ ID NO: 262 (or a variant thereof), an HCDR2 comprising the amino acid sequence set forth in SEQ ID NO: 263 (or a variant thereof), and an HCDR3 comprising the amino acid sequence set forth in SEQ ID NO: 264 (or a variant thereof); and a LCVR that comprises: an LCDR1 comprising the amino acid sequence set forth in SEQ ID NO: 267 (or a variant thereof), an LCDR2 comprising the amino acid sequence set forth in SEQ ID NO: 268 (or a variant thereof), and an LCDR3 comprising the amino acid sequence set forth in SEQ ID NO: 269 (or a variant thereof); (k) a HCVR that comprises: an HCDR1 comprising the amino acid sequence set forth in SEQ ID NO: 272 (or a variant thereof), an HCDR2 comprising the amino acid sequence set forth in SEQ ID NO: 273 (or a variant thereof), and an HCDR3 comprising the amino acid sequence set forth in SEQ ID NO: 274 (or a variant thereof); and a LCVR that comprises: an LCDR1 comprising the amino acid sequence set forth in SEQ ID NO: 277 (or a variant thereof), an LCDR2 comprising the amino acid sequence set forth in SEQ ID NO: 278 (or a variant thereof), and an LCDR3 comprising the amino acid sequence set forth in SEQ ID NO: 279 (or a variant thereof); (1) a HCVR that comprises: an HCDR1 comprising the amino acid sequence set forth in SEQ ID NO: 282 (or a variant thereof), an HCDR2 comprising the amino acid sequence set forth in SEQ ID NO: 283 (or a variant thereof), and an HCDR3 comprising the amino acid sequence set forth in SEQ ID NO: 284 (or a variant thereof); and a LCVR that comprises: an LCDR1 comprising the amino acid sequence set forth in SEQ ID NO: 287 (or a variant thereof), an LCDR2 comprising the amino acid sequence set forth in SEQ ID NO: 288 (or a variant thereof), and an LCDR3 comprising the amino acid sequence set forth in SEQ ID NO: 289 (or a variant thereof); (m) a HCVR that comprises: an HCDR1 comprising the amino acid sequence set forth in SEQ ID NO: 292 (or a variant thereof), an HCDR2 comprising the amino acid sequence set forth in SEQ ID NO: 293 (or a variant thereof), and an HCDR3 comprising the amino acid sequence set forth in SEQ ID NO: 294 (or a variant thereof); and a LCVR that comprises: an LCDR1 comprising the amino acid sequence set forth in SEQ ID NO: 297 (or a variant thereof), an LCDR2 comprising the amino acid sequence set forth in SEQ ID NO: 298 (or a variant thereof), and an LCDR3 comprising the amino acid sequence set forth in SEQ ID NO: 299 (or a variant thereof); (n) a HCVR that comprises: an HCDR1 comprising the amino acid sequence set forth in SEQ ID NO: 302 (or a variant thereof), an HCDR2 comprising the amino acid sequence set forth in SEQ ID NO: 303 (or a variant thereof), and an HCDR3 comprising the amino acid sequence set forth in SEQ ID NO: 304 (or a variant thereof); and a LCVR that comprises: an LCDR1 comprising the amino acid sequence set forth in SEQ ID NO: 307 (or a variant thereof), an LCDR2 comprising the amino acid sequence set forth in SEQ ID NO: 308 (or a variant thereof), and an LCDR3 comprising the amino acid sequence set forth in SEQ ID NO: 309 (or a variant thereof); (o) a HCVR that comprises: an HCDR1 comprising the amino acid sequence set forth in SEQ ID NO: 312 (or a variant thereof), an HCDR2 comprising the amino acid sequence set forth in SEQ ID NO: 313 (or a variant thereof), and an HCDR3 comprising the amino acid sequence set forth in SEQ ID NO: 314 (or a variant thereof); and a LCVR that comprises: an LCDR1 comprising the amino acid sequence set forth in SEQ ID NO: 317 (or a variant thereof), an LCDR2 comprising the amino acid sequence set forth in SEQ ID NO: 318 (or a variant thereof), and an LCDR3 comprising the amino acid sequence set forth in SEQ ID NO: 319 (or a variant thereof); (p) a HCVR that comprises: an HCDR1 comprising the amino acid sequence set forth in SEQ ID NO: 322 (or a variant thereof), an HCDR2 comprising the amino acid sequence set forth in SEQ ID NO: 323 (or a variant thereof), and an HCDR3 comprising the amino acid sequence set forth in SEQ ID NO: 324 (or a variant thereof); and a LCVR that comprises: an LCDR1 comprising the amino acid sequence set forth in SEQ ID NO: 327 (or a variant thereof), an LCDR2 comprising the amino acid sequence set forth in SEQ ID NO: 328 (or a variant thereof), and an LCDR3 comprising the amino acid sequence set forth in SEQ ID NO: 329 (or a variant thereof); (q) a HCVR that comprises: an HCDR1 comprising the amino acid sequence set forth in SEQ ID NO: 332 (or a variant thereof), an HCDR2 comprising the amino acid sequence set forth in SEQ ID NO: 333 (or a variant thereof), and an HCDR3 comprising the amino acid sequence set forth in SEQ ID NO: 334 (or a variant thereof); and a LCVR that comprises: an LCDR1 comprising the amino acid sequence set forth in SEQ ID NO: 337 (or a variant thereof), an LCDR2 comprising the amino acid sequence set forth in SEQ ID NO: 338 (or a variant thereof), and an LCDR3 comprising the amino acid sequence set forth in SEQ ID NO: 339 (or a variant thereof); (r) a HCVR that comprises: an HCDR1 comprising the amino acid sequence set forth in SEQ ID NO: 342 (or a variant thereof), an HCDR2 comprising the amino acid sequence set forth in SEQ ID NO: 343 (or a variant thereof), and an HCDR3 comprising the amino acid sequence set forth in SEQ ID NO: 344 (or a variant thereof); and a LCVR that comprises: an LCDR1 comprising the amino acid sequence set forth in SEQ ID NO: 347 (or a variant thereof), an LCDR2 comprising the amino acid sequence set forth in SEQ ID NO: 348 (or a variant thereof), and an LCDR3 comprising the amino acid sequence set forth in SEQ ID NO: 349 (or a variant thereof); (s) a HCVR that comprises: an HCDR1 comprising the amino acid sequence set forth in SEQ ID NO: 352 (or a variant thereof), an HCDR2 comprising the amino acid sequence set forth in SEQ ID NO: 353 (or a variant thereof), and an HCDR3 comprising the amino acid sequence set forth in SEQ ID NO: 354 (or a variant thereof); and a LCVR that comprises: an LCDR1 comprising the amino acid sequence set forth in SEQ ID NO: 357 (or a variant thereof), an LCDR2 comprising the amino acid sequence set forth in SEQ ID NO: 358 (or a variant thereof), and an LCDR3 comprising the amino acid sequence set forth in SEQ ID NO: 359 (or a variant thereof); (t) a HCVR that comprises: an HCDR1 comprising the amino acid sequence set forth in SEQ ID NO: 362 (or a variant thereof), an HCDR2 comprising the amino acid sequence set forth in SEQ ID NO: 363 (or a variant thereof), and an HCDR3 comprising the amino acid sequence set forth in SEQ ID NO: 364 (or a variant thereof); and a LCVR that comprises: an LCDR1 comprising the amino acid sequence set forth in SEQ ID NO: 367 (or a variant thereof), an LCDR2 comprising the amino acid sequence set forth in SEQ ID NO: 368 (or a variant thereof), and an LCDR3 comprising the amino acid sequence set forth in SEQ ID NO: 369 (or a variant thereof); (u) a HCVR that comprises: an HCDR1 comprising the amino acid sequence set forth in SEQ ID NO: 372 (or a variant thereof), an HCDR2 comprising the amino acid sequence set forth in SEQ ID NO: 373 (or a variant thereof), and an HCDR3 comprising the amino acid sequence set forth in SEQ ID NO: 374 (or a variant thereof); and a LCVR that comprises: an LCDR1 comprising the amino acid sequence set forth in SEQ ID NO: 377 (or a variant thereof), an LCDR2 comprising the amino acid sequence set forth in SEQ ID NO: 378 (or a variant thereof), and an LCDR3 comprising the amino acid sequence set forth in SEQ ID NO: 379 (or a variant thereof); (v) a HCVR that comprises: an HCDR1 comprising the amino acid sequence set forth in SEQ ID NO: 382 (or a variant thereof), an HCDR2 comprising the amino acid sequence set forth in SEQ ID NO: 383 (or a variant thereof), and an HCDR3 comprising the amino acid sequence set forth in SEQ ID NO: 384 (or a variant thereof); and a LCVR that comprises: an LCDR1 comprising the amino acid sequence set forth in SEQ ID NO: 387 (or a variant thereof), an LCDR2 comprising the amino acid sequence set forth in SEQ ID NO: 388 (or a variant thereof), and an LCDR3 comprising the amino acid sequence set forth in SEQ ID NO: 389 (or a variant thereof); (w) a HCVR that comprises: an HCDR1 comprising the amino acid sequence set forth in SEQ ID NO: 392 (or a variant thereof), an HCDR2 comprising the amino acid sequence set forth in SEQ ID NO: 393 (or a variant thereof), and an HCDR3 comprising the amino acid sequence set forth in SEQ ID NO: 394 (or a variant thereof); and a LCVR that comprises: an LCDR1 comprising the amino acid sequence set forth in SEQ ID NO: 397 (or a variant thereof), an LCDR2 comprising the amino acid sequence set forth in SEQ ID NO: 398 (or a variant thereof), and an LCDR3 comprising the amino acid sequence set forth in SEQ ID NO: 399 (or a variant thereof); (x) a HCVR that comprises: an HCDR1 comprising the amino acid sequence set forth in SEQ ID NO: 402 (or a variant thereof), an HCDR2 comprising the amino acid sequence set forth in SEQ ID NO: 403 (or a variant thereof), and an HCDR3 comprising the amino acid sequence set forth in SEQ ID NO: 404 (or a variant thereof); and a LCVR that comprises: an LCDR1 comprising the amino acid sequence set forth in SEQ ID NO: 407 (or a variant thereof), an LCDR2 comprising the amino acid sequence set forth in SEQ ID NO: 408 (or a variant thereof), and an LCDR3 comprising the amino acid sequence set forth in SEQ ID NO: 409 (or a variant thereof); (y) a HCVR that comprises: an HCDR1 comprising the amino acid sequence set forth in SEQ ID NO: 412 (or a variant thereof), an HCDR2 comprising the amino acid sequence set forth in SEQ ID NO: 413 (or a variant thereof), and an HCDR3 comprising the amino acid sequence set forth in SEQ ID NO: 414 (or a variant thereof); and a LCVR that comprises: an LCDR1 comprising the amino acid sequence set forth in SEQ ID NO: 417 (or a variant thereof), an LCDR2 comprising the amino acid sequence set forth in SEQ ID NO: 418 (or a variant thereof), and an LCDR3 comprising the amino acid sequence set forth in SEQ ID NO: 419 (or a variant thereof); (z) a HCVR that comprises: an HCDR1 comprising the amino acid sequence set forth in SEQ ID NO: 422 (or a variant thereof), an HCDR2 comprising the amino acid sequence set forth in SEQ ID NO: 423 (or a variant thereof), and an HCDR3 comprising the amino acid sequence set forth in SEQ ID NO: 424 (or a variant thereof); and a LCVR that comprises: an LCDR1 comprising the amino acid sequence set forth in SEQ ID NO: 427 (or a variant thereof), an LCDR2 comprising the amino acid sequence set forth in SEQ ID NO: 428 (or a variant thereof), and an LCDR3 comprising the amino acid sequence set forth in SEQ ID NO: 429 (or a variant thereof); (aa) a HCVR that comprises: an HCDR1 comprising the amino acid sequence set forth in SEQ ID NO: 432 (or a variant thereof), an HCDR2 comprising the amino acid sequence set forth in SEQ ID NO: 433 (or a variant thereof), and an HCDR3 comprising the amino acid sequence set forth in SEQ ID NO: 434 (or a variant thereof); and a LCVR that comprises: an LCDR1 comprising the amino acid sequence set forth in SEQ ID NO: 437 (or a variant thereof), an LCDR2 comprising the amino acid sequence set forth in SEQ ID NO: 438 (or a variant thereof), and an LCDR3 comprising the amino acid sequence set forth in SEQ ID NO: 439 (or a variant thereof); (ab) a HCVR that comprises: an HCDR1 comprising the amino acid sequence set forth in SEQ ID NO: 442 (or a variant thereof), an HCDR2 comprising the amino acid sequence set forth in SEQ ID NO: 443 (or a variant thereof), and an HCDR3 comprising the amino acid sequence set forth in SEQ ID NO: 444 (or a variant thereof); and a LCVR that comprises: an LCDR1 comprising the amino acid sequence set forth in SEQ ID NO: 447 (or a variant thereof), an LCDR2 comprising the amino acid sequence set forth in SEQ ID NO: 448 (or a variant thereof), and an LCDR3 comprising the amino acid sequence set forth in SEQ ID NO: 449 (or a variant thereof); (ac) a HCVR that comprises: an HCDR1 comprising the amino acid sequence set forth in SEQ ID NO: 452 (or a variant thereof), an HCDR2 comprising the amino acid sequence set forth in SEQ ID NO: 453 (or a variant thereof), and an HCDR3 comprising the amino acid sequence set forth in SEQ ID NO: 454 (or a variant thereof); and a LCVR that comprises: an LCDR1 comprising the amino acid sequence set forth in SEQ ID NO: 457 (or a variant thereof), an LCDR2 comprising the amino acid sequence set forth in SEQ ID NO: 458 (or a variant thereof), and an LCDR3 comprising the amino acid sequence set forth in SEQ ID NO: 459 (or a variant thereof); (ad) a HCVR that comprises: an HCDR1 comprising the amino acid sequence set forth in SEQ ID NO: 462 (or a variant thereof), an HCDR2 comprising the amino acid sequence set forth in SEQ ID NO: 463 (or a variant thereof), and an HCDR3 comprising the amino acid sequence set forth in SEQ ID NO: 464 (or a variant thereof); and a LCVR that comprises: an LCDR1 comprising the amino acid sequence set forth in SEQ ID NO: 467 (or a variant thereof), an LCDR2 comprising the amino acid sequence set forth in SEQ ID NO: 468 (or a variant thereof), and an LCDR3 comprising the amino acid sequence set forth in SEQ ID NO: 469 (or a variant thereof); (ae) a HCVR that comprises: an HCDR1 comprising the amino acid sequence set forth in SEQ ID NO: 472 (or a variant thereof), an HCDR2 comprising the amino acid sequence set forth in SEQ ID NO: 473 (or a variant thereof), and an HCDR3 comprising the amino acid sequence set forth in SEQ ID NO: 474 (or a variant thereof); and a LCVR that comprises: an LCDR1 comprising the amino acid sequence set forth in SEQ ID NO: 477 (or a variant thereof), an LCDR2 comprising the amino acid sequence set forth in SEQ ID NO: 478 (or a variant thereof), and an LCDR3 comprising the amino acid sequence set forth in SEQ ID NO: 479 (or a variant thereof); and/or (af) a HCVR that comprises: an HCDR1 comprising the amino acid sequence set forth in SEQ ID NO: 482 (or a variant thereof), an HCDR2 comprising the amino acid sequence set forth in SEQ ID NO: 483 (or a variant thereof), and an HCDR3 comprising the amino acid sequence set forth in SEQ ID NO: 484 (or a variant thereof); and a LCVR that comprises: an LCDR1 comprising the amino acid sequence set forth in SEQ ID NO: 487 (or a variant thereof), an LCDR2 comprising the amino acid sequence set forth in SEQ ID NO: 488 (or a variant thereof), and an LCDR3 comprising the amino acid sequence set forth in SEQ ID NO: 489 (or a variant thereof). A variant refers to a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein.

In another example, the anti-TfR antibody or antigen-binding fragment thereof or scFv can comprise: (w) a HCVR that comprises: an HCDR1 comprising the amino acid sequence set forth in SEQ ID NO: 392 (or a variant thereof), an HCDR2 comprising the amino acid sequence set forth in SEQ ID NO: 393 (or a variant thereof), and an HCDR3 comprising the amino acid sequence set forth in SEQ ID NO: 394 (or a variant thereof); and a LCVR that comprises: an LCDR1 comprising the amino acid sequence set forth in SEQ ID NO: 397 (or a variant thereof), an LCDR2 comprising the amino acid sequence set forth in SEQ ID NO: 398 (or a variant thereof), and an LCDR3 comprising the amino acid sequence set forth in SEQ ID NO: 399 (or a variant thereof); or (y) a HCVR that comprises: an HCDR1 comprising the amino acid sequence set forth in SEQ ID NO: 412 (or a variant thereof), an HCDR2 comprising the amino acid sequence set forth in SEQ ID NO: 413 (or a variant thereof), and an HCDR3 comprising the amino acid sequence set forth in SEQ ID NO: 414 (or a variant thereof); and a LCVR that comprises: an LCDR1 comprising the amino acid sequence set forth in SEQ ID NO: 417 (or a variant thereof), an LCDR2 comprising the amino acid sequence set forth in SEQ ID NO: 418 (or a variant thereof), and an LCDR3 comprising the amino acid sequence set forth in SEQ ID NO: 419 (or a variant thereof). In another example, the anti-TfR antibody or antigen-binding fragment thereof or scFv can comprise a HCVR that comprises: an HCDR1 comprising the amino acid sequence set forth in SEQ ID NO: 392 (or a variant thereof), an HCDR2 comprising the amino acid sequence set forth in SEQ ID NO: 393 (or a variant thereof), and an HCDR3 comprising the amino acid sequence set forth in SEQ ID NO: 394 (or a variant thereof); and a LCVR that comprises: an LCDR1 comprising the amino acid sequence set forth in SEQ ID NO: 397 (or a variant thereof), an LCDR2 comprising the amino acid sequence set forth in SEQ ID NO: 398 (or a variant thereof), and an LCDR3 comprising the amino acid sequence set forth in SEQ ID NO: 399 (or a variant thereof). In another example, the anti-TfR antibody or antigen-binding fragment thereof or scFv can comprise a HCVR that comprises: an HCDR1 comprising the amino acid sequence set forth in SEQ ID NO: 412 (or a variant thereof), an HCDR2 comprising the amino acid sequence set forth in SEQ ID NO: 413 (or a variant thereof), and an HCDR3 comprising the amino acid sequence set forth in SEQ ID NO: 414 (or a variant thereof); and a LCVR that comprises: an LCDR1 comprising the amino acid sequence set forth in SEQ ID NO: 417 (or a variant thereof), an LCDR2 comprising the amino acid sequence set forth in SEQ ID NO: 418 (or a variant thereof), and an LCDR3 comprising the amino acid sequence set forth in SEQ ID NO: 419 (or a variant thereof).

In another example, the anti-TfR antibody or antigen-binding fragment thereof or scFv can comprise: (i) a HCVR that comprises the amino acid sequence set forth in SEQ ID NO: 171 or 680 (or a variant thereof); and a LCVR that comprises the amino acid sequence set forth in SEQ ID NO: 176 (or a variant thereof); (ii) a HCVR that comprises the amino acid sequence set forth in SEQ ID NO: 181 or 681 (or a variant thereof); and a LCVR that comprises the amino acid sequence set forth in SEQ ID NO: 186 (or a variant thereof); (iii) a HCVR that comprises the amino acid sequence set forth in SEQ ID NO: 191 or 682 (or a variant thereof); and a LCVR that comprises the amino acid sequence set forth in SEQ ID NO: 196 (or a variant thereof); (iv) a HCVR that comprises the amino acid sequence set forth in SEQ ID NO: 201 (or a variant thereof); and a LCVR that comprises the amino acid sequence set forth in SEQ ID NO: 206 or 683 (or a variant thereof); (v) a HCVR that comprises the amino acid sequence set forth in SEQ ID NO: 211 (or a variant thereof); and a LCVR that comprises the amino acid sequence set forth in SEQ ID NO: 216 or 684 (or a variant thereof); (vi) a HCVR that comprises the amino acid sequence set forth in SEQ ID NO: 221 or 685 (or a variant thereof); and a LCVR that comprises the amino acid sequence set forth in SEQ ID NO: 226 or 686 (or a variant thereof); (vii) a HCVR that comprises the amino acid sequence set forth in SEQ ID NO: 231 or 687 (or a variant thereof); and a LCVR that comprises the amino acid sequence set forth in SEQ ID NO: 236 or 688 (or a variant thereof); (viii) a HCVR that comprises the amino acid sequence set forth in SEQ ID NO: 241 or 689 (or a variant thereof); and a LCVR that comprises the amino acid sequence set forth in SEQ ID NO: 246 or 690 (or a variant thereof); (ix) a HCVR that comprises the amino acid sequence set forth in SEQ ID NO: 251 (or a variant thereof); and a LCVR that comprises the amino acid sequence set forth in SEQ ID NO: 256 (or a variant thereof); (x) a HCVR that comprises the amino acid sequence set forth in SEQ ID NO: 261 or 691 (or a variant thereof); and a LCVR that comprises the amino acid sequence set forth in SEQ ID NO: 266 (or a variant thereof); (xi) a HCVR that comprises the amino acid sequence set forth in SEQ ID NO: 271 (or a variant thereof); and a LCVR that comprises the amino acid sequence set forth in SEQ ID NO: 276 (or a variant thereof); (xii) a HCVR that comprises the amino acid sequence set forth in SEQ ID NO: 281 or 692 (or a variant thereof); and a LCVR that comprises the amino acid sequence set forth in SEQ ID NO: 286 or 693 (or a variant thereof); (xiii) a HCVR that comprises the amino acid sequence set forth in SEQ ID NO: 291 (or a variant thereof); and a LCVR that comprises the amino acid sequence set forth in SEQ ID NO: 296 (or a variant thereof); (xiv) a HCVR that comprises the amino acid sequence set forth in SEQ ID NO: 301 (or a variant thereof); and a LCVR that comprises the amino acid sequence set forth in SEQ ID NO: 306 (or a variant thereof); (xv) a HCVR that comprises the amino acid sequence set forth in SEQ ID NO: 311 or 694 (or a variant thereof); and a LCVR that comprises the amino acid sequence set forth in SEQ ID NO: 316 or 695 (or a variant thereof); (xvi) a HCVR that comprises the amino acid sequence set forth in SEQ ID NO: 321 (or a variant thereof); and a LCVR that comprises the amino acid sequence set forth in SEQ ID NO: 326 (or a variant thereof); (xvii) a HCVR that comprises the amino acid sequence set forth in SEQ ID NO: 331 or 696 (or a variant thereof); and a LCVR that comprises the amino acid sequence set forth in SEQ ID NO: 336 (or a variant thereof); (xviii) a HCVR that comprises the amino acid sequence set forth in SEQ ID NO: 341 (or a variant thereof); and a LCVR that comprises the amino acid sequence set forth in SEQ ID NO: 346 (or a variant thereof); (xix) a HCVR that comprises the amino acid sequence set forth in SEQ ID NO: 351 or 697 (or a variant thereof); and a LCVR that comprises the amino acid sequence set forth in SEQ ID NO: 356 or 698 (or a variant thereof); (xx) a HCVR that comprises the amino acid sequence set forth in SEQ ID NO: 361 or 699 (or a variant thereof); and a LCVR that comprises the amino acid sequence set forth in SEQ ID NO: 366 (or a variant thereof); (xxi) a HCVR that comprises the amino acid sequence set forth in SEQ ID NO: 371 or 700 (or a variant thereof); and a LCVR that comprises the amino acid sequence set forth in SEQ ID NO: 376 (or a variant thereof); (xxii) a HCVR that comprises the amino acid sequence set forth in SEQ ID NO: 381 (or a variant thereof); and a LCVR that comprises the amino acid sequence set forth in SEQ ID NO: 386 (or a variant thereof); (xxiii) a HCVR that comprises the amino acid sequence set forth in SEQ ID NO: 391 (or a variant thereof); and a LCVR that comprises the amino acid sequence set forth in SEQ ID NO: 396 (or a variant thereof); (xxiv) a HCVR that comprises the amino acid sequence set forth in SEQ ID NO: 401 (or a variant thereof); and a LCVR that comprises the amino acid sequence set forth in SEQ ID NO: 406 (or a variant thereof); (xxv) a HCVR that comprises the amino acid sequence set forth in SEQ ID NO: 411 (or a variant thereof); and a LCVR that comprises the amino acid sequence set forth in SEQ ID NO: 416 (or a variant thereof); (xxvi) a HCVR that comprises the amino acid sequence set forth in SEQ ID NO: 421 or 701 (or a variant thereof); and a LCVR that comprises the amino acid sequence set forth in SEQ ID NO: 426 (or a variant thereof); (xxvii) a HCVR that comprises the amino acid sequence set forth in SEQ ID NO: 431 (or a variant thereof); and a LCVR that comprises the amino acid sequence set forth in SEQ ID NO: 436 (or a variant thereof); (xxviii) a HCVR that comprises the amino acid sequence set forth in SEQ ID NO: 441 (or a variant thereof); and a LCVR that comprises the amino acid sequence set forth in SEQ ID NO: 446 (or a variant thereof); (xxix) a HCVR that comprises the amino acid sequence set forth in SEQ ID NO: 451 (or a variant thereof); and a LCVR that comprises the amino acid sequence set forth in SEQ ID NO: 456 (or a variant thereof); (xxx) a HCVR that comprises the amino acid sequence set forth in SEQ ID NO: 461 (or a variant thereof); and a LCVR that comprises the amino acid sequence set forth in SEQ ID NO: 466 (or a variant thereof); (xxxi) a HCVR that comprises the amino acid sequence set forth in SEQ ID NO: 471 or 702 (or a variant thereof); and a LCVR that comprises the amino acid sequence set forth in SEQ ID NO: 476 or 632 (or a variant thereof); and/or (xxxii) a HCVR that comprises the amino acid sequence set forth in SEQ ID NO: 481 (or a variant thereof); and a LCVR that comprises the amino acid sequence set forth in SEQ ID NO: 486 or 703 (or a variant thereof), optionally wherein the HCVR and LCVR are linked by a linker (e.g., that comprises an amino acid sequence, e.g., about 10 amino acids in length, for example, 1, 2, 3, 4, 5, 6, 7, 8, 8, or 10 repeats of Gly₄Ser (SEQ ID NO: 537). A variant refers to a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein.

In another example, the anti-TfR antibody or antigen-binding fragment thereof or scFv can comprise: (xxiii) a HCVR that comprises the amino acid sequence set forth in SEQ ID NO: 391 (or a variant thereof); and a LCVR that comprises the amino acid sequence set forth in SEQ ID NO: 396 (or a variant thereof); or (xxv) a HCVR that comprises the amino acid sequence set forth in SEQ ID NO: 411 (or a variant thereof); and a LCVR that comprises the amino acid sequence set forth in SEQ ID NO: 416 (or a variant thereof). In another example, the anti-TfR antibody or antigen-binding fragment thereof or scFv can comprise a HCVR that comprises the amino acid sequence set forth in SEQ ID NO: 391 (or a variant thereof); and a LCVR that comprises the amino acid sequence set forth in SEQ ID NO: 396 (or a variant thereof). In another example, the anti-TfR antibody or antigen-binding fragment thereof or scFv can comprise a HCVR that comprises the amino acid sequence set forth in SEQ ID NO: 411 (or a variant thereof); and a LCVR that comprises the amino acid sequence set forth in SEQ ID NO: 416 (or a variant thereof).

Examples of polynucleotides encoding anti-TfR antibodies or antigen-binding fragments thereof or scFvs are provided in Table 2 and include: (1) a polynucleotide encoding a HCVR that comprises the nucleotide sequence set forth in SEQ ID NO: 170, and a LCVR that comprises the nucleotide sequence set forth in SEQ ID NO: 175; (2) a polynucleotide encoding a HCVR that comprises the nucleotide sequence set forth in SEQ ID NO: 180, and a LCVR that comprises the nucleotide sequence set forth in SEQ ID NO: 185; (3) a polynucleotide encoding a HCVR that comprises the nucleotide sequence set forth in SEQ ID NO: 190, and a LCVR that comprises the nucleotide sequence set forth in SEQ ID NO: 195; (4) a polynucleotide encoding a HCVR that comprises the nucleotide sequence set forth in SEQ ID NO: 200, and a LCVR that comprises the nucleotide sequence set forth in SEQ ID NO: 205; (5) a polynucleotide encoding a HCVR that comprises the nucleotide sequence set forth in SEQ ID NO: 210, and a LCVR that comprises the nucleotide sequence set forth in SEQ ID NO: 215; (6) a polynucleotide encoding a HCVR that comprises the nucleotide sequence set forth in SEQ ID NO: 220, and a LCVR that comprises the nucleotide sequence set forth in SEQ ID NO: 225; (7) a polynucleotide encoding a HCVR that comprises the nucleotide sequence set forth in SEQ ID NO: 230, and a LCVR that comprises the nucleotide sequence set forth in SEQ ID NO: 235; (8) a polynucleotide encoding a HCVR that comprises the nucleotide sequence set forth in SEQ ID NO: 240, and a LCVR that comprises the nucleotide sequence set forth in SEQ ID NO: 245; (9) a polynucleotide encoding a HCVR that comprises the nucleotide sequence set forth in SEQ ID NO: 250, and a LCVR that comprises the nucleotide sequence set forth in SEQ ID NO: 255; (10) a polynucleotide encoding a HCVR that comprises the nucleotide sequence set forth in SEQ ID NO: 260, and a LCVR that comprises the nucleotide sequence set forth in SEQ ID NO: 265; (11) a polynucleotide encoding a HCVR that comprises the nucleotide sequence set forth in SEQ ID NO: 270, and a LCVR that comprises the nucleotide sequence set forth in SEQ ID NO: 275; (12) a polynucleotide encoding a HCVR that comprises the nucleotide sequence set forth in SEQ ID NO: 280, and a LCVR that comprises the nucleotide sequence set forth in SEQ ID NO: 285; (13) a polynucleotide encoding a HCVR that comprises the nucleotide sequence set forth in SEQ ID NO: 290, and a LCVR that comprises the nucleotide sequence set forth in SEQ ID NO: 295; (14) a polynucleotide encoding a HCVR that comprises the nucleotide sequence set forth in SEQ ID NO: 300, and a LCVR that comprises the nucleotide sequence set forth in SEQ ID NO: 305; (15) a polynucleotide encoding a HCVR that comprises the nucleotide sequence set forth in SEQ ID NO: 310, and a LCVR that comprises the nucleotide sequence set forth in SEQ ID NO: 315; (16) a polynucleotide encoding a HCVR that comprises the nucleotide sequence set forth in SEQ ID NO: 320, and a LCVR that comprises the nucleotide sequence set forth in SEQ ID NO: 325; (17) a polynucleotide encoding a HCVR that comprises the nucleotide sequence set forth in SEQ ID NO: 330, and a LCVR that comprises the nucleotide sequence set forth in SEQ ID NO: 335; (18) a polynucleotide encoding a HCVR that comprises the nucleotide sequence set forth in SEQ ID NO: 340, and a LCVR that comprises the nucleotide sequence set forth in SEQ ID NO: 345; (19) a polynucleotide encoding a HCVR that comprises the nucleotide sequence set forth in SEQ ID NO: 350, and a LCVR that comprises the nucleotide sequence set forth in SEQ ID NO: 355; (20) a polynucleotide encoding a HCVR that comprises the nucleotide sequence set forth in SEQ ID NO: 360, and a LCVR that comprises the nucleotide sequence set forth in SEQ ID NO: 365; (21) a polynucleotide encoding a HCVR that comprises the nucleotide sequence set forth in SEQ ID NO: 370, and a LCVR that comprises the nucleotide sequence set forth in SEQ ID NO: 375; (22) a polynucleotide encoding a HCVR that comprises the nucleotide sequence set forth in SEQ ID NO: 380, and a LCVR that comprises the nucleotide sequence set forth in SEQ ID NO: 385; (23) a polynucleotide encoding a HCVR that comprises the nucleotide sequence set forth in SEQ ID NO: 390, and a LCVR that comprises the nucleotide sequence set forth in SEQ ID NO: 395; (24) a polynucleotide encoding a HCVR that comprises the nucleotide sequence set forth in SEQ ID NO: 400, and a LCVR that comprises the nucleotide sequence set forth in SEQ ID NO: 405; (25) a polynucleotide encoding a HCVR that comprises the nucleotide sequence set forth in SEQ ID NO: 410, and a LCVR that comprises the nucleotide sequence set forth in SEQ ID NO: 415; (26) a polynucleotide encoding a HCVR that comprises the nucleotide sequence set forth in SEQ ID NO: 420, and a LCVR that comprises the nucleotide sequence set forth in SEQ ID NO: 425; (27) a polynucleotide encoding a HCVR that comprises the nucleotide sequence set forth in SEQ ID NO: 430, and a LCVR that comprises the nucleotide sequence set forth in SEQ ID NO: 435; (28) a polynucleotide encoding a HCVR that comprises the nucleotide sequence set forth in SEQ ID NO: 440, and a LCVR that comprises the nucleotide sequence set forth in SEQ ID NO: 445; (29) a polynucleotide encoding a HCVR that comprises the nucleotide sequence set forth in SEQ ID NO: 450, and a LCVR that comprises the nucleotide sequence set forth in SEQ ID NO: 455; (30) a polynucleotide encoding a HCVR that comprises the nucleotide sequence set forth in SEQ ID NO: 460, and a LCVR that comprises the nucleotide sequence set forth in SEQ ID NO: 465; (31) a polynucleotide encoding a HCVR that comprises the nucleotide sequence set forth in SEQ ID NO: 470, and a LCVR that comprises the nucleotide sequence set forth in SEQ ID NO: 475; or (32) a polynucleotide encoding a HCVR that comprises the nucleotide sequence set forth in SEQ ID NO: 480, and a LCVR that comprises the nucleotide sequence set forth in SEQ ID NO: 485, wherein the HCVR and LCVR are in either order.

In an embodiment, an anti-hTfR scFv, in V_{L}-(Gly₄Ser)₃(SEQ ID NO: 616)-V_{H} format (Gly₄Ser=SEQ ID NO: 537), comprises the amino acid sequence set forth in any one of SEQ ID NOS: 492-523. Also contemplated are such fusions that are in the format V_{H}-(Gly₄Ser)₃(SEQ ID NO: 616)-V_{L} (Gly₄Ser=SEQ ID NO: 537).

In an embodiment, the antigen-binding fragment comprises an scFv. In an embodiment, the scFv comprises the amino acid sequence set forth in SEQ ID NO: 508 (or a variant thereof) or comprises the amino acid sequence set forth in SEQ ID NO: 505 (or a variant thereof). In an embodiment, the scFv comprises the amino acid sequence set forth in SEQ ID NO: 508 (or a variant thereof). In an embodiment, the antigen-binding fragment comprises an scFv. In an embodiment, the scFv comprises the amino acid sequence set forth in SEQ ID NO: 505 (or a variant thereof).

In an embodiment, the TfR-binding delivery domain can be an scFv. In an embodiment, the anti-TfR scFv protein is (or the anti-TfR scFv protein comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 494, 503, 505, and 508 (and, e.g., retaining TfR-binding activity). Optionally, the anti-TfR scFv protein is (or the anti-TfR scFv protein comprises a sequence) at least 95%, at least 96%, at least 97%, at least 98%, at least 99%, at least 99.5%, or 100% identical to any one of SEQ ID NOS: 494, 503, 505, and 508 (and, e.g., retaining TfR-binding activity). Optionally, the anti-TfR scFv protein is (or the anti-TfR scFv protein comprises a sequence) at least 99%, at least 99.5%, or 100% identical to any one of SEQ ID NOS: 494, 503, 505, and 508 (and, e.g., retaining TfR-binding activity). Optionally, the anti-TfR scFv protein comprises the sequence set forth in any one of SEQ ID NOS: 494, 503, 505, and 508. Optionally, the anti-TfR scFv protein consists essentially of the sequence set forth in any one of SEQ ID NOS: 494, 503, 505, and 508. Optionally, the anti-TfR scFv protein consists of the sequence set forth in any one of SEQ ID NOS: 494, 503, 505, and 508.

In an embodiment, the anti-TfR scFv protein is (or the anti-TfR scFv protein comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 508 (and, e.g., retaining TfR-binding activity). Optionally, the anti-TfR scFv protein is (or the anti-TfR scFv protein comprises a sequence) at least 95%, at least 96%, at least 97%, at least 98%, at least 99%, at least 99.5%, or 100% identical to SEQ ID NO: 508 (and, e.g., retaining TfR-binding activity). Optionally, the anti-TfR scFv protein is (or the anti-TfR scFv protein comprises a sequence) at least 99%, at least 99.5%, or 100% identical to SEQ ID NO: 508 (and, e.g., retaining TfR-binding activity). Optionally, the anti-TfR scFv protein comprises the sequence set forth in SEQ ID NO: 508. Optionally, the anti-TfR scFv protein consists essentially of the sequence set forth in SEQ ID NO: 508. Optionally, the anti-TfR scFv protein consists of the sequence set forth in SEQ ID NO: 508.

In an embodiment, the anti-TfR scFv protein is (or the anti-TfR scFv protein comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 505 (and, e.g., retaining TfR-binding activity). Optionally, the anti-TfR scFv protein is (or the anti-TfR scFv protein comprises a sequence) at least 95%, at least 96%, at least 97%, at least 98%, at least 99%, at least 99.5%, or 100% identical to SEQ ID NO: 505 (and, e.g., retaining TfR-binding activity). Optionally, the anti-TfR scFv protein is (or the anti-TfR scFv protein comprises a sequence) at least 99%, at least 99.5%, or 100% identical to SEQ ID NO: 505 (and, e.g., retaining TfR-binding activity). Optionally, the anti-TfR scFv protein comprises the sequence set forth in SEQ ID NO: 505. Optionally, the anti-TfR scFv protein consists essentially of the sequence set forth in SEQ ID NO: 505. Optionally, the anti-TfR scFv protein consists of the sequence set forth in SEQ ID NO: 505.

In an embodiment, the multidomain therapeutic protein comprises the amino acid sequence set forth in SEQ ID NO: 737 (or a variant thereof) or comprises the amino acid sequence set forth in SEQ ID NO: 739 (or a variant thereof). In an embodiment, the multidomain therapeutic protein comprises the amino acid sequence set forth in SEQ ID NO: 737 (or a variant thereof). In an embodiment, the multidomain therapeutic protein comprises the amino acid sequence set forth in SEQ ID NO: 739 (or a variant thereof).

In another example, the TfR-binding delivery domain can be a Fab fragment (e.g., that binds specifically to human transferrin receptor). Fab fragments typically contain one complete light chain, VL and constant light domain, e.g., kappa (e.g., RTVAAPSVFIFPPSDEQLKSGTASVVCLLNNFYPREAKVQWKVDNALQSGNSQESVTEQDSKDS TYSLSSTLTLSKADYEKHKVYACEVTHQGLSSPVTKSFNRGEC (SEQ ID NO: 538)) and the V_{H} and IgG1 CH1 portion (e.g., ASTKGPSVFPLAPSSKSTSGGTAALGCLVKDYFPEPVTVSWNSGALTSGVHTFPAVLQSSGLYS LSSVVTVPSSSLGTQTYICNVNHKPSNTKVDKKVEPKSCDKTH (SEQ ID NO: 539)) or IgG4 CH1 (e.g., ASTKGPSVFPLAPCSRSTSESTAALGCLVKDYFPEPVTVSWNSGALTSGVHTFPAVLQSSGLYS LSSVVTVPSSSLGTKTYTCNVDHKPSNTKVDKRVESKYGPPLLQGSG (SEQ ID NO: 611) or ASTKGPSVFPLAPCSRSTSESTAALGCLVKDYFPEPVTVSWNSGALTSGVHTFPAVLQSSGLYS LSSVVTVPSSSLGTKTYTCNVDHKPSNTKVDKRVESKYGPP (SEQ ID NO: 631)) of one heavy chain. Fab fragment antibodies can be generated by papain digestion of whole IgG antibodies to remove the entire Fc fragment, including the hinge region. In some embodiments, a Fab protein can comprise a heavy chain upstream of a light chain. In some embodiments, a Fab protein can comprise a light chain upstream of a heavy chain. In one example, the antibody or antigen-binding fragment thereof or Fab protein can comprise: (1) a heavy chain variable region (HCVR) that comprises the amino acid sequence set forth in SEQ ID NO: 171 or 680, or a heavy chain variable region that includes HCDR1, HCDR2 and HCDR3 of such a HCVR-linked to the CH1 domain- and a light chain variable region (LCVR) that comprises the amino acid sequence set forth in SEQ ID NO: 176, or LCDR1, LCDR2 and LCDR3 of such a LCVR-linked to the CL domain; (2) a heavy chain variable region (HCVR) that comprises the amino acid sequence set forth in SEQ ID NO: 181 or 681, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 186, or LCDR1, LCDR2 and LCDR3 of such a LCVR-linked to the CL domain; (3) a heavy chain variable region (HCVR) that comprises the amino acid sequence set forth in SEQ ID NO: 191 or 682, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 196, or LCDR1, LCDR2 and LCDR3 of such a LCVR-linked to the CL domain; (4) a heavy chain variable region (HCVR) that comprises the amino acid sequence set forth in SEQ ID NO: 201, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 206 or 683, or LCDR1, LCDR2 and LCDR3 of such a LCVR-linked to the CL domain; (5) a heavy chain variable region (HCVR) that comprises the amino acid sequence set forth in SEQ ID NO: 211, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 216 or 684, or LCDR1, LCDR2 and LCDR3 of such a LCVR-linked to the CL domain; (6) a heavy chain variable region (HCVR) that comprises the amino acid sequence set forth in SEQ ID NO: 221 or 685, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 226 or 686, or LCDR1, LCDR2 and LCDR3 of such a LCVR-linked to the CL domain; (7) a heavy chain variable region (HCVR) that comprises the amino acid sequence set forth in SEQ ID NO: 231 or 687, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 236 or 688, or LCDR1, LCDR2 and LCDR3 of such a LCVR-linked to the CL domain; (8) a heavy chain variable region (HCVR) that comprises the amino acid sequence set forth in SEQ ID NO: 241 or 689, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 246 or 690, or LCDR1, LCDR2 and LCDR3 of such a LCVR-linked to the CL domain; (9) a heavy chain variable region (HCVR) that comprises the amino acid sequence set forth in SEQ ID NO: 251, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 256, or LCDR1, LCDR2 and LCDR3 of such a LCVR-linked to the CL domain; (10) a heavy chain variable region (HCVR) that comprises the amino acid sequence set forth in SEQ ID NO: 261 or 691, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 266, or LCDR1, LCDR2 and LCDR3 of such a LCVR-linked to the CL domain; (11) a heavy chain variable region (HCVR) that comprises the amino acid sequence set forth in SEQ ID NO: 271, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 276, or LCDR1, LCDR2 and LCDR3 of such a LCVR-linked to the CL domain; (12) a heavy chain variable region (HCVR) that comprises the amino acid sequence set forth in SEQ ID NO: 281 or 692, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 286 or 693, or LCDR1, LCDR2 and LCDR3 of such a LCVR-linked to the CL domain; (13) a heavy chain variable region (HCVR) that comprises the amino acid sequence set forth in SEQ ID NO: 291, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 296, or LCDR1, LCDR2 and LCDR3 of such a LCVR-linked to the CL domain; (14) a heavy chain variable region (HCVR) that comprises the amino acid sequence set forth in SEQ ID NO: 301, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 306, or LCDR1, LCDR2 and LCDR3 of such a LCVR-linked to the CL domain; (15) a heavy chain variable region (HCVR) that comprises the amino acid sequence set forth in SEQ ID NO: 311 or 694, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 316 or 695, or LCDR1, LCDR2 and LCDR3 of such a LCVR-linked to the CL domain; (16) a heavy chain variable region (HCVR) that comprises the amino acid sequence set forth in SEQ ID NO: 321, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 326, or LCDR1, LCDR2 and LCDR3 of such a LCVR-linked to the CL domain; (17) a heavy chain variable region (HCVR) that comprises the amino acid sequence set forth in SEQ ID NO: 331 or 696, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 336, or LCDR1, LCDR2 and LCDR3 of such a LCVR-linked to the CL domain; (18) a heavy chain variable region (HCVR) that comprises the amino acid sequence set forth in SEQ ID NO: 341, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 346, or LCDR1, LCDR2 and LCDR3 of such a LCVR-linked to the CL domain; (19) a heavy chain variable region (HCVR) that comprises the amino acid sequence set forth in SEQ ID NO: 351 or 697, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 356 or 698, or LCDR1, LCDR2 and LCDR3 of such a LCVR-linked to the CL domain; (20) a heavy chain variable region (HCVR) that comprises the amino acid sequence set forth in SEQ ID NO: 361 or 699, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 366, or LCDR1, LCDR2 and LCDR3 of such a LCVR-linked to the CL domain; (21) a heavy chain variable region (HCVR) that comprises the amino acid sequence set forth in SEQ ID NO: 371 or 700, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 376, or LCDR1, LCDR2 and LCDR3 of such a LCVR-linked to the CL domain; (22) a heavy chain variable region (HCVR) that comprises the amino acid sequence set forth in SEQ ID NO: 381, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 386, or LCDR1, LCDR2 and LCDR3 of such a LCVR-linked to the CL domain; (23) a heavy chain variable region (HCVR) that comprises the amino acid sequence set forth in SEQ ID NO: 391, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 396, or LCDR1, LCDR2 and LCDR3 of such a LCVR-linked to the CL domain; (24) a heavy chain variable region (HCVR) that comprises the amino acid sequence set forth in SEQ ID NO: 401, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 406, or LCDR1, LCDR2 and LCDR3 of such a LCVR-linked to the CL domain; (25) a heavy chain variable region (HCVR) that comprises the amino acid sequence set forth in SEQ ID NO: 411, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 416, or LCDR1, LCDR2 and LCDR3 of such a LCVR-linked to the CL domain; (26) a heavy chain variable region (HCVR) that comprises the amino acid sequence set forth in SEQ ID NO: 421 or 701, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 426, or LCDR1, LCDR2 and LCDR3 of such a LCVR-linked to the CL domain; (27) a heavy chain variable region (HCVR) that comprises the amino acid sequence set forth in SEQ ID NO: 431, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 436, or LCDR1, LCDR2 and LCDR3 of such a LCVR-linked to the CL domain; (28) a heavy chain variable region (HCVR) that comprises the amino acid sequence set forth in SEQ ID NO: 441, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 446, or LCDR1, LCDR2 and LCDR3 of such a LCVR-linked to the CL domain; (29) a heavy chain variable region (HCVR) that comprises the amino acid sequence set forth in SEQ ID NO: 451, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 456, or LCDR1, LCDR2 and LCDR3 of such a LCVR-linked to the CL domain; (30) a heavy chain variable region (HCVR) that comprises the amino acid sequence set forth in SEQ ID NO: 461, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 466, or LCDR1, LCDR2 and LCDR3 of such a LCVR-linked to the CL domain; (31) a heavy chain variable region (HCVR) that comprises the amino acid sequence set forth in SEQ ID NO: 471 or 702, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 476 or 632, or LCDR1, LCDR2 and LCDR3 of such a LCVR-linked to the CL domain; and/or (32) a heavy chain variable region (HCVR) that comprises the amino acid sequence set forth in SEQ ID NO: 481, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 486 or 703, or LCDR1, LCDR2 and LCDR3 of such a LCVR-linked to the CL domain. For example, the CH1 can be SEQ ID NO: 539 or 611.

In one example, the antibody or antigen-binding fragment thereof or Fab protein can comprise: (23) a heavy chain variable region (HCVR) that comprises the amino acid sequence set forth in SEQ ID NO: 391, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 396, or LCDR1, LCDR2 and LCDR3 of such a LCVR-linked to the CL domain; or (25) a heavy chain variable region (HCVR) that comprises the amino acid sequence set forth in SEQ ID NO: 411, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 416, or LCDR1, LCDR2 and LCDR3 of such a LCVR-linked to the CL domain. In one example, the Fab protein can comprise a heavy chain variable region (HCVR) that comprises the amino acid sequence set forth in SEQ ID NO: 391, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 396, or LCDR1, LCDR2 and LCDR3 of such a LCVR-linked to the CL domain. In one example, the Fab protein can comprise a heavy chain variable region (HCVR) that comprises the amino acid sequence set forth in SEQ ID NO: 411, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 416, or LCDR1, LCDR2 and LCDR3 of such a LCVR-linked to the CL domain.

In one example, the antibody or antigen-binding fragment thereof or Fab protein can comprise: (1) a light chain that comprises the amino acid sequence set forth in SEQ ID NO: 540 and a heavy chain that comprises the amino acid sequence set forth in SEQ ID NO: 541 (31874B); (2) a light chain that comprises the amino acid sequence set forth in SEQ ID NO: 542 and a heavy chain that comprises the amino acid sequence set forth in SEQ ID NO: 543 (31863B); (3) a light chain that comprises the amino acid sequence set forth in SEQ ID NO: 544 and a heavy chain that comprises the amino acid sequence set forth in SEQ ID NO: 545 (69348); (4) a light chain that comprises the amino acid sequence set forth in SEQ ID NO: 546 and a heavy chain that comprises the amino acid sequence set forth in SEQ ID NO: 547 (69340); (5) a light chain that comprises the amino acid sequence set forth in SEQ ID NO: 548 and a heavy chain that comprises the amino acid sequence set forth in SEQ ID NO: 549 (69331); (6) a light chain that comprises the amino acid sequence set forth in SEQ ID NO: 550 and a heavy chain that comprises the amino acid sequence set forth in SEQ ID NO: 551 (69332); (7) a light chain that comprises the amino acid sequence set forth in SEQ ID NO: 552 and a heavy chain that comprises the amino acid sequence set forth in SEQ ID NO: 553 (69326); (8) a light chain that comprises the amino acid sequence set forth in SEQ ID NO: 554 and a heavy chain that comprises the amino acid sequence set forth in SEQ ID NO: 555 (69329); (9) a light chain that comprises the amino acid sequence set forth in SEQ ID NO: 556 and a heavy chain that comprises the amino acid sequence set forth in SEQ ID NO: 557 (69323); (10) a light chain that comprises the amino acid sequence set forth in SEQ ID NO: 558 and a heavy chain that comprises the amino acid sequence set forth in SEQ ID NO: 559 (69305); (11) a light chain that comprises the amino acid sequence set forth in SEQ ID NO: 560 and a heavy chain that comprises the amino acid sequence set forth in SEQ ID NO: 561 (69307); (12) a light chain that comprises the amino acid sequence set forth in SEQ ID NO: 562 and a heavy chain that comprises the amino acid sequence set forth in SEQ ID NO: 563 (12795B); (13) a light chain that comprises the amino acid sequence set forth in SEQ ID NO: 564 and a heavy chain that comprises the amino acid sequence set forth in SEQ ID NO: 565 or SEQ ID NO: 604 or SEQ ID NO: 633 (12798B); (14) a light chain that comprises the amino acid sequence set forth in SEQ ID NO: 566 and a heavy chain that comprises the amino acid sequence set forth in SEQ ID NO: 567 or SEQ ID NO: 605 or SEQ ID NO: 634 (12799B); (15) a light chain that comprises the amino acid sequence set forth in SEQ ID NO: 568 and a heavy chain that comprises the amino acid sequence set forth in SEQ ID NO: 569 (12801B); (16) a light chain that comprises the amino acid sequence set forth in SEQ ID NO: 570 and a heavy chain that comprises the amino acid sequence set forth in SEQ ID NO: 571 (12802B); (17) a light chain that comprises the amino acid sequence set forth in SEQ ID NO: 572 and a heavy chain that comprises the amino acid sequence set forth in SEQ ID NO: 573 (12808B); (18) a light chain that comprises the amino acid sequence set forth in SEQ ID NO: 574 and a heavy chain that comprises the amino acid sequence set forth in SEQ ID NO: 575 (12812B); (19) a light chain that comprises the amino acid sequence set forth in SEQ ID NO: 576 and a heavy chain that comprises the amino acid sequence set forth in SEQ ID NO: 577 (12816B); (20) a light chain that comprises the amino acid sequence set forth in SEQ ID NO: 578 and a heavy chain that comprises the amino acid sequence set forth in SEQ ID NO: 579 (12833B); (21) a light chain that comprises the amino acid sequence set forth in SEQ ID NO: 580 and a heavy chain that comprises the amino acid sequence set forth in SEQ ID NO: 581 (12834B); (22) a light chain that comprises the amino acid sequence set forth in SEQ ID NO: 582 and a heavy chain that comprises the amino acid sequence set forth in SEQ ID NO: 583 (12835B); (23) a light chain that comprises the amino acid sequence set forth in SEQ ID NO: 584 and a heavy chain that comprises the amino acid sequence set forth in SEQ ID NO: 585 or SEQ ID NO: 606 or SEQ ID NO: 635 (12847B); (24) a light chain that comprises the amino acid sequence set forth in SEQ ID NO: 586 and a heavy chain that comprises the amino acid sequence set forth in SEQ ID NO: 587 (12848B); (25) a light chain that comprises the amino acid sequence set forth in SEQ ID NO: 588 and a heavy chain that comprises the amino acid sequence set forth in SEQ ID NO: 589 or SEQ ID NO: 607 or SEQ ID NO: 636 (12843B); (26) a light chain that comprises the amino acid sequence set forth in SEQ ID NO: 590 and a heavy chain that comprises the amino acid sequence set forth in SEQ ID NO: 591 (12844B); (27) a light chain that comprises the amino acid sequence set forth in SEQ ID NO: 592 and a heavy chain that comprises the amino acid sequence set forth in SEQ ID NO: 593 or SEQ ID NO: 608 or SEQ ID NO: 637 (12845B); (28) a light chain that comprises the amino acid sequence set forth in SEQ ID NO: 594 and a heavy chain that comprises the amino acid sequence set forth in SEQ ID NO: 595 or SEQ ID NO: 609 or SEQ ID NO: 638 (12839B); (29) a light chain that comprises the amino acid sequence set forth in SEQ ID NO: 596 and a heavy chain that comprises the amino acid sequence set forth in SEQ ID NO: 597 (12841B); (30) a light chain that comprises the amino acid sequence set forth in SEQ ID NO: 598 and a heavy chain that comprises the amino acid sequence set forth in SEQ ID NO: 599 (12850B); (31) a light chain that comprises the amino acid sequence set forth in SEQ ID NO: 600 and a heavy chain that comprises the amino acid sequence set forth in SEQ ID NO: 601 (69261); or (32) a light chain that comprises the amino acid sequence set forth in SEQ ID NO: 602 and a heavy chain that comprises the amino acid sequence set forth in SEQ ID NO: 603 (69263).

In one example, the antibody or antigen-binding fragment thereof or Fab protein can comprise: (23) a light chain that comprises the amino acid sequence set forth in SEQ ID NO: 584 and a heavy chain that comprises the amino acid sequence set forth in SEQ ID NO: 585 or SEQ ID NO: 606 or SEQ ID NO: 635 (12847B); or (25) a light chain that comprises the amino acid sequence set forth in SEQ ID NO: 588 and a heavy chain that comprises the amino acid sequence set forth in SEQ ID NO: 589 or SEQ ID NO: 607 or SEQ ID NO: 636 (12843B). In one example, the antibody or antigen-binding fragment thereof or Fab protein can comprise a light chain that comprises the amino acid sequence set forth in SEQ ID NO: 584 and a heavy chain that comprises the amino acid sequence set forth in SEQ ID NO: 585 or SEQ ID NO: 606 or SEQ ID NO: 635 (12847B). In one example, the antibody or antigen-binding fragment thereof or Fab protein can comprise a light chain that comprises the amino acid sequence set forth in SEQ ID NO: 584 and a heavy chain that comprises the amino acid sequence set forth in SEQ ID NO: 635 (12847B). In one example, the antibody or antigen-binding fragment thereof or Fab protein can comprise a light chain that comprises the amino acid sequence set forth in SEQ ID NO: 588 and a heavy chain that comprises the amino acid sequence set forth in SEQ ID NO: 589 or SEQ ID NO: 607 or SEQ ID NO: 636 (12843B).

In an embodiment, the antigen-binding fragment comprises a Fab protein. In an embodiment, the Fab protein comprises the amino acid sequences set forth in SEQ ID NO: 584 and 635 (or variants thereof) or comprises the amino acid sequences set forth in SEQ ID NO: 588 and 636 (or variants thereof). In an embodiment, the Fab protein comprises the amino acid sequences set forth in SEQ ID NO: 584 and 635 (or variants thereof). In an embodiment, the Fab protein comprises the amino acid sequences set forth in SEQ ID NO: 588 and 636 (or variants thereof).

“31874B”; “31863B”; “69348”; “69340”; “69331”; “69332”; “69326”; “69329”; “69323”; “69305”; “69307”; “12795B”; “12798B”; “12799B”; “12801B”; “12802B”; “12808B”; “12812B”; “12816B”; “12833B”; “12834B”; “12835B”; “12847B”; “12848B”; “12843B”; “12844B”; “12845B”; “12839B”; “12841B”; “12850B”; “69261”; and “69263” refer to anti-TfR:GAA fusion proteins, e.g., anti-TfR scFv:GAA or anti-TfR Fab:GAA, comprising a light chain variable region comprising the amino acid sequence set forth in SEQ ID NO: 176, 186, 196, 206, 683, 216, 684, 226, 686, 236, 688, 246, 690, 256, 266, 276, 286, 693, 296, 306, 316, 695, 326, 336, 346, 356, 698, 366, 376, 386, 396, 406, 416, 426, 436, 446, 456, 466, 476, 632, 486, or 703 (or a variant thereof), and a heavy chain variable region comprising the amino acid sequence set forth in SEQ ID NO: 171, 680, 181, 681, 191, 682, 201, 211, 221, 685, 231, 687, 241, 689, 251, 261, 691, 271, 281, 692, 291, 301, 311, 694, 321, 331, 696, 341, 351, 697, 361, 699, 371, 700, 381, 391, 401, 411, 421, 701, 431, 441, 451, 461, 471, 702, or 481 (or a variant thereof); which, in the case of an scFv, can be fused together (in either order), e.g., by a peptide linker (e.g., (G₄S)₃(SEQ ID NO: 616)) (G₄S=SEQ ID NO: 537), respectively; or that comprise a V_{H} that comprises the CDRs thereof (CDR-H1 (or a variant thereof), CDR-H2 (or a variant thereof) and CDR-H3 (or a variant thereof)) and/or a V_{L} that comprises the CDRs thereof (CDR-L1 (or a variant thereof), CDR-L2 (or a variant thereof) and CDR-L3 (or a variant thereof)), wherein the V_{H} fused to the V_{L} or the V_{L} fused to the V_{H}, in the case of an scFv, can be fused, e.g., by a peptide linker (e.g., (G₄S)₂(SEQ ID NO: 617)) (G₄S=SEQ ID NO: 537), to a GAA polypeptide.

In some embodiments, the anti-TfR antigen-binding protein described herein comprises a humanized antibody or antigen binding fragment thereof, murine antibody or antigen binding fragment thereof, chimeric antibody or antigen binding fragment thereof, monoclonal antibody or antigen binding fragment thereof (e.g., monovalent Fab′, divalent Fab2, F(ab)′3 fragments, single-chain variable fragment (scFv), bis-scFv, (scFv)2, diabody, bivalent antibody, one-armed antibody, minibody, nanobody, triabody, tetrabody, disulfide stabilized Fv protein (dsFv), single-domain antibody (sdAb), Ig NAR, camelid antibody or antigen binding fragment thereof, bispecific antibody or biding fragment thereof, (e.g., bisscFv, or a bi-specific T-cell engager (BiTE)), trispecific antibody (e.g., F(ab)′3 fragments or a triabody), or a chemically modified derivative thereof. In some embodiments, the anti-TfR antigen-binding protein can be bivalent. In some embodiments, the anti-TfR antigen-binding protein can be monovalent (e.g., one-arm antibody).

The term “humanized antibody,” as used herein, includes antibodies in which CDR sequences derived from the germline of another mammalian species, such as a mouse, have been grafted onto human framework sequences, or otherwise modified to increase their similarity to antibody variants produced naturally in humans.

In some cases, the anti-TfR antigen-binding protein is an antibody which comprises one or more mutations in a framework region, e.g., in the CH1 domain, CH2 domain, CH3 domain, hinge region, or a combination thereof. In some embodiments, the one or more mutations are to stabilize the antibody and/or to increase half-life. In some embodiments, the one or more mutations are to modulate Fc receptor interactions, to reduce or eliminate Fc effector functions such as FcγR, antibody-dependent cell-mediated cytotoxicity (ADCC), or complement-dependent cytotoxicity (CDC). In additional embodiments, the one or more mutations are to modulate glycosylation.

In some embodiments, one, two or more mutations (e.g., amino acid substitutions) are introduced into the Fc region of an antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody, such as serum half-life, complement fixation, Fc receptor binding and/or antigen-dependent cellular cytotoxicity. In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., PCT Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo. In some embodiments, the Fc region comprises a mutation at residue position L234, L235, or a combination thereof. In some embodiments, the mutations comprise L234 and L235. In some embodiments, the mutations comprise L234A and L235A.

The anti-TfR antibodies and antigen-binding fragments described herein may be modified after translation, e.g., glycosylated.

For example, antibodies and antigen-binding fragments described herein may be glycosylated (e.g., N-glycosylated and/or O-glycosylated) or aglycosylated. Typically, antibodies and antigen-binding fragments are glycosylated at the conserved residue N297 of the IgG Fc domain. Some antibodies and fragments include one or more additional glycosylation sites in a variable region. In an embodiment, the glycosylation site is in the following context: FN₂₉₇S or YN₂₉₇S.

In an embodiment, said glycosylation is any one or more of three different N-glycan types: high mannose, complex and/or hybrid that are found on IgGs with their respective linkage. Complex and hybrid types exist with core fucosylation, addition of a fucose residue to the innermost N-acetylglucosamine, and without core fucosylation.

In some cases, the anti-TfR antigen-binding protein is an aglycosylated antibody, i.e., an antibody that does not comprise a glycosylation sequence that might interfere with a transglutamination reaction, for instance an antibody that does not have a saccharide group at N180 and/or N297 on one or more heavy chains. In particular embodiments, an antibody heavy chain has an N180 mutation. In other words, the antibody is mutated to no longer have an asparagine residue at position 180 according to the EU numbering system as disclosed by Kabat et al. In particular embodiments, an antibody heavy chain has an N180Q mutation. In particular embodiments, an antibody heavy chain has an N297 mutation. In particular embodiments, an antibody heavy chain has an N297Q or an N297D mutation. Antibodies comprising such above-described mutations can be prepared by site-directed mutagenesis to remove or disable a glycosylation sequence or by site-directed mutagenesis to insert a glutamine residue at site apart from any interfering glycosylation site or any other interfering structure. Such antibodies also can be isolated from natural or artificial sources. Aglycosylated antibodies also include antibodies comprising a T299 or S298P or other mutations, or combinations of mutations that result in a lack of glycosylation.

In some cases, the antigen-binding protein is a deglycosylated antibody, i.e., an antibody in which a saccharide group at is removed to facilitate transglutaminase-mediated conjugation. Saccharides include, but are not limited to, N-linked oligosaccharides. In some embodiments, deglycosylation is performed at residue N180. In some embodiments, deglycosylation is performed at residue N297. In some embodiments, removal of saccharide groups is accomplished enzymatically, included but not limited to via PNGase.

In an embodiment, an antibody or fragment described herein is afucosylated.

The antibodies and antigen-binding fragments described herein may also be post-translationally modified in other ways including, for example: Glu or Gln cyclization at N-terminus; Loss of positive N-terminal charge; Lys variants at C-terminus; Deamidation (Asn to Asp); Isomerization (Asp to isoAsp); Deamidation (Gln to Glu); Oxidation (Cys, His, Met, Tyr, Trp); and/or Disulfide bond heterogeneity (Shuffling, thioether and trisulfide formation).

In some embodiments, an antibody disclosed herein Q295 which can be native to the antibody heavy chain sequence. In some embodiments, an antibody heavy chain disclosed herein may comprise Q295. In some embodiments, an antibody heavy chain disclosed herein may comprise Q295 and an amino acid substitution N297D.

According to certain embodiments of the present disclosure, anti-TfR antibodies and antigen-binding fragments are provided comprising an Fc domain comprising one or more mutations which enhance or diminish antibody binding to the FcRn receptor, e.g., at acidic pH as compared to neutral pH. For example, the present disclosure includes anti-TfR antibodies comprising a mutation in the CH2 or a CH3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal.

Non-limiting examples of such Fc modifications include, e.g., a modification at position:
250 (e.g., E or Q);
250 and 428 (e.g., L or F);
252 (e.g., L/Y/F/W or T),
254 (e.g., S or T), and/or
256 (e.g., S/R/Q/E/D or T);
   and/or a modification at position:
428 and/or 433 (e.g., H/L/R/S/P/Q or K), and/or
434 (e.g., A, W, H, F or Y);
   and/or a modification at position:
250 and/or 428;
   and/or a modification at position:
307 or 308 (e.g., 308F, V308F), and/or
434.

In an embodiment, the modification comprises:
a 428L (e.g., M428L) and 434S (e.g., N434S) modification;
a 428L, 259I (e.g., V259I), and 308F (e.g., V308F) modification;
a 433K (e.g., H433K) and a 434 (e.g., 434Y) modification;
a 252, 254, and 256 (e.g., 252Y, 254T, and 256E) modification;
a 250Q and 428L modification (e.g., T250Q and M428L); and/or
a 307 and/or 308 modification (e.g., 308F or 308P).

In yet another embodiment, the modification comprises a 265A (e.g., D265A) and/or a 297A (e.g., N297A) modification.

For example, the present disclosure includes anti-TfR antibodies comprising an Fc domain comprising one or more pairs or groups of mutations selected from the group consisting of:
250Q and 248L (e.g., T250Q and M248L);
252Y, 254T and 256E (e.g., M252Y, S254T and T256E);
2571 and 3111 (e.g., P2571 and Q3111);
2571 and 434H (e.g., P2571 and N434H);
376V and 434H (e.g., D376V and N434H);
307A, 380A and 434A (e.g., T307A, E380A and N434A);
428L and 434S (e.g., M428L and N434S); and
433K and 434F (e.g., H433K and N434F).

In an embodiment, the heavy chain constant domain is gamma4 comprising an S228P and/or S108P mutation. See Angal et al., A single amino acid substitution abolishes the heterogeneity of chimeric mouse/human (IgG4) antibody, Mol Immunol. 1993 January; 30(1):105-108.

All possible combinations of the foregoing Fc domain mutations, and other mutations within the antibody variable domains disclosed herein, are contemplated within the scope of the present disclosure.

The anti-TfR antibodies described herein may comprise a modified Fc domain having reduced effector function. As used herein, a “modified Fc domain having reduced effector function” means any Fc portion of an immunoglobulin that has been modified, mutated, truncated, etc., relative to a wild-type, naturally occurring Fc domain such that a molecule comprising the modified Fc exhibits a reduction in the severity or extent of at least one effect selected from the group consisting of cell killing (e.g., ADCC and/or CDC), complement activation, phagocytosis and opsonization, relative to a comparator molecule comprising the wild-type, naturally occurring version of the Fc portion. In certain embodiments, a “modified Fc domain having reduced effector function” is an Fc domain with reduced or attenuated binding to an Fc receptor (e.g., FcγR).

In certain embodiments, the modified Fc domain is a variant IgG1 Fc or a variant IgG4 Fc comprising a substitution in the hinge region. For example, a modified Fc for use in the context of the present disclosure may comprise a variant IgG1 Fc wherein at least one amino acid of the IgG1 Fc hinge region is replaced with the corresponding amino acid from the IgG2 Fc hinge region. Alternatively, a modified Fc for use in the context of the present disclosure may comprise a variant IgG4 Fc wherein at least one amino acid of the IgG4 Fc hinge region is replaced with the corresponding amino acid from the IgG2 Fc hinge region. Non-limiting, exemplary modified Fc regions that can be used in the context of the present disclosure are set forth in US Patent Application Publication No. 2014/0243504, the disclosure of which is hereby incorporated by reference in its entirety, as well as any functionally equivalent variants of the modified Fc regions set forth therein.

Also provided herein are antigen-binding proteins, antibodies or antigen-binding fragments, comprising a HCVR set forth herein and a chimeric heavy chain constant (CH) region, wherein the chimeric CH region comprises segments derived from the CH regions of more than one immunoglobulin isotype. For example, the antibodies of the disclosure may comprise a chimeric CH region comprising part or all of a CH2 domain derived from a human IgG1, human IgG2 or human IgG4 molecule, combined with part or all of a CH3 domain derived from a human IgG1, human IgG2 or human IgG4 molecule. According to certain embodiments, the antibodies provided herein comprise a chimeric CH region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric CH region as described herein may, in certain embodiments, exhibit modified Fc effector functions without adversely affecting the therapeutic or pharmacokinetic properties of the antibody. See, e.g., WO2014/022540.

Other modified Fc domains and Fc modifications that can be used in the context of the present disclosure include any of the modifications as set forth in US2014/0171623; U.S. Pat. No. 8,697,396; US2014/0134162; WO2014/043361, the disclosures of which are hereby incorporated by reference in their entireties. Methods of constructing antibodies or other antigen-binding fusion proteins comprising a modified Fc domain as described herein are known in the art.

In some embodiments, the anti-TfR antibodies and antigen-binding fragments described herein comprise an Fc domain comprising one or more mutations in the CH2 and/or CH3 regions that generate a separate TfR binding site.

In an embodiment, the CH2 region comprises one or more amino acid mutations, or a combination thereof, selected from the following: a) position 47 is Glu, Gly, Gln, Ser, Ala, Asn, Tyr, or Trp; position 49 is Ile, Val, Asp, Glu, Thr, Ala, or Tyr; position 56 is Asp, Pro, Met, Leu, Ala, Asn, or Phe; position 58 is Arg, Ser, Ala, or Gly; position 59 is Tyr, Trp, Arg, or Val; position 60 is Glu; position 61 is Trp or Tyr; position 62 is Gln, Tyr, His, Ile, Phe, Val, or Asp; and position 63 is Leu, Trp, Arg, Asn, Tyr, or Val; b) position 39 is Pro, Phe, Ala, Met, or Asp; position 40 is Gln, Pro, Arg, Lys, Ala, Ile, Leu, Glu, Asp, or Tyr; position 41 is Thr, Ser, Gly, Met, Val, Phe, Trp, or Leu; position 42 is Pro, Val, Ala, Thr, or Asp; position 43 is Pro, Val, or Phe; position 44 is Trp, Gln, Thr, or Glu; position 68 is Glu, Val, Thr, Leu, or Trp; position 70 is Tyr, His, Val, or Asp; position 71 is Thr, His, Gln, Arg, Asn, or Val; and position 72 is Tyr, Asn, Asp, Ser, or Pro; c) position 41 is Val or Asp; position 42 is Pro, Met, or Asp; position 43 is Pro or Trp; position 44 is Arg, Trp, Glu, or Thr; position 45 is Met, Tyr, or Trp; position 65 is Len or Trp; position 66 is Thr, Val, Ile, or Lys; position 67 is Ser, Lys, Ala, or Leu; position 69 is His, Leu, or Pro; and position 73 is Val or Trp; or d) position 45 is Trp, Val, Ile, or Ala; position 47 is Trp or Gly; position 49 is Tyr, Arg, or Glu; position 95 is Ser, Arg, or Gln; position 97 is Val, Ser, or Phe; position 99 is Ile, Ser, or Trp; position 102 is Trp, Thr, Ser, Arg, or Asp; position 103 is Trp; and position 104 is Ser, Lys, Arg, or Val; wherein the substitutions and the positions are determined with reference to amino acids 4-113 of

(SEQ ID NO: 807)
PCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKC
KVSNKALPAPIEKTISKAKGQPREPQVYTLPPSRDELTKNQVSLTCL
VKGFYPSDIAVEWESNGQPENNYKTTPPVLDSDGSFFLYSKLTVDKS
RWQQGNVFSCSVMHEALHNHYTQKSLSLSPGK.

In an embodiment, the CH3 region comprises one or more amino acid mutations, or a combination thereof, selected from the following: position 153 is Trp, Leu, or Glu; position 157 is Tyr or Phe; position 159 is Thr; position 160 is Glu; position 161 is Trp; position 162 is Ser, Ala, Val, or Asn; position 163 is Ser or Asn; position 186 is Thr or Ser; position 188 is Glu or Ser; position 189 is Glu; and position 194 is Phe; or b) position 118 is Phe or Ile; position 119 is Asp, Glu, Gly, Ala, or Lys; position 120 is Tyr, Met, Leu, Ile, or Asp; position 122 is Thr or Ala; position 210 is Gly; position 211 is Phe; position 212 is His, Tyr, Ser, or Phe; and position 213 is Asp; wherein the substitutions and the positions are determined with reference to amino acids 114-220 of SEQ ID NO: 807.

In some embodiments, the CH3 region comprises one or more mutations, or a combination thereof, selected from the following: position 384 is Leu, Tyr, Met, or Val; position 386 is Leu, Thr, His, or Pro; position 387 is Val, Pro, or an acidic amino acid; position 388 is Trp; position 389 is Val, Ser, or Ala; position 413 is Glu, Ala, Ser, Leu, Thr, or Pro; position 416 is Thr or an acidic amino acid; and position 421 is Trp, Tyr, His, or Phe, according to EU numbering. In an embodiment, the CH3 region comprises one or more amino acid mutations, or a combination thereof, selected from the following: a) position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe.

In some embodiments, the CH3 region comprises one or more mutations, or a combination thereof, selected from the following: a) Phe at position 382, Tyr at position 383, Asp at position 384, Asp at position 385, Ser at position 386, Lys at position 387, Len at position 388, Thr at position 389, Pro at position 419, Arg at position 420, Gly at position 421, Len at position 422, Ala at position 424, Glu at position 426, Tyr at position 438, Len at position 440, Gly at position 442, and Glu at position 443; b) Phe at position 382, Tyr at position 383, Gly at position 384, N at position 385, Ala at position 386, Lys at position 387, Thr at position 389, Len at position 422, Ala at position 424, Glu at position 426, Tyr at position 438, Len at position 440; c) Phe at position 382, Tyr at position 383, Glu at position 384, Ala at position 385, Lys at position 387, Len at position 388, Len at position 422, Ala at position 424, Glu at position 426, Tyr at position 438, Len at position 440; d) Phe at position 382, Glu at position 384, Ser at position 386, Lys at position 387, Thr at position 389, Len at position 422, Ala at position 424, Glu at position 426, Tyr at position 438, Len at position 440; e) Phe at position 382, Gly at position 384, Ala at position 385, Lys at position 387, Ser at position 389, Len at position 422, Ala at position 424, Glu at position 426, Tyr at position 438, Len at position 440; f) Phe at position 382, Gly at position 384, Ala at position 385, Lys at position 387, Len at position 388, Thr at position 389, Len at position 422, Ala at position 424, Glu at position 426, Tyr at position 438, Len at position 440; wherein the positions are determined according to EU numbering.

Additional mutations in CH2 and/or CH3 regions that can introduce non-native TfR binding sites into the antigen-binding proteins descried herein include those described in US Patent Application Publication Nos. 2020/0223935, 2020/0369746, 2021/0130485, 2022/0017634; and PCT Application Publications Nos. WO2023/279099, WO2023/114499 and WO2023/114510, which are incorporated herein by reference in their entireties.

The TfR-binding delivery domain coding sequences in the constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a TfR-binding delivery domain coding sequence in a construct disclosed herein has one or more cryptic splice sites mutated or removed. In another example, a TfR-binding delivery domain coding sequence in a construct disclosed herein has all identified cryptic splice sites mutated or removed. In another example, a TfR-binding delivery domain coding sequence in a construct disclosed herein has one or more CpG dinucleotides removed (i.e., is CpG depleted). In another example, a TfR-binding delivery domain coding sequence in a construct disclosed herein has all CpG dinucleotides removed (i.e., is fully CpG depleted). In another example, a TfR-binding delivery domain coding sequence in a construct disclosed herein is codon optimized (e.g., codon optimized for expression in a human or mammal). In a specific example, a CDTfR63-binding delivery domain coding sequence in a construct disclosed herein has one or more CpG dinucleotides removed (i.e., is CpG depleted) and has one or more cryptic splice sites mutated or removed. In another specific example, a TfR-binding delivery domain coding sequence in a construct disclosed herein has all CpG dinucleotides removed and has one or more or all identified cryptic splice sites mutated or removed. In another specific example, a TfR-binding delivery domain coding sequence in a construct disclosed herein has one or more CpG dinucleotides removed (i.e., is CpG depleted) and is codon optimized (e.g., codon optimized for expression in a human or mammal). In another specific example, a TfR-binding delivery domain coding sequence in a construct disclosed herein has all CpG dinucleotides removed (i.e., is fully CpG depleted) and is codon optimized (e.g., codon optimized for expression in a human or mammal).

Various anti-TfR scFv coding sequences are provided. In one example, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 492-523 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to any one of SEQ ID NOS: 492-523 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to any one of SEQ ID NOS: 492-523 (and, e.g., retaining TfR-binding activity). Optionally, the anti-TfR scFv coding sequence in the above examples encodes an anti-TfR scFv protein comprising the sequence set forth in any one of SEQ ID NOS: 492-523. Optionally, the anti-TfR scFv coding sequence in the above examples encodes an anti-TfR scFv protein consisting essentially of the sequence set forth in any one of SEQ ID NOS: 492-523. Optionally, the anti-TfR scFv coding sequence in the above examples encodes an anti-TfR scFv protein consisting of the sequence set forth in any one of SEQ ID NOS: 492-523.

Various anti-TfR scFv coding sequences are provided. In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 524-536. In another example, the anti-TfR scFv coding sequence is (or comprises a sequence) at least 95%, at least 96%, at least 97%, at least 98%, at least 99%, at least 99.5%, or 100% identical to any one of SEQ ID NOS: 524-536. In another example, the anti-TfR scFv coding sequence is (or comprises a sequence) at least 99%, at least 99.5%, or 100% identical to any one of SEQ ID NOS: 524-536. In another example, the anti-TfR scFv coding sequence comprises the sequence set forth in any one of SEQ ID NOS: 524-536. In another example, the anti-TfR scFv coding sequence consists essentially of the sequence set forth in any one of SEQ ID NOS: 524-536. In another example, the anti-TfR scFv coding sequence consists of the sequence set forth in any one of SEQ ID NOS: 524-536. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 494, 503, 505, and 508 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to any one of SEQ ID NOS: 494, 503, 505, and 508 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to any one of SEQ ID NOS: 494, 503, 505, and 508 (and, e.g., retaining TfR-binding activity). Optionally, the anti-TfR scFv coding sequence in the above examples encodes an anti-TfR scFv protein comprising the sequence set forth in any one of SEQ ID NOS: 494, 503, 505, and 508. Optionally, the anti-TfR scFv coding sequence in the above examples encodes an anti-TfR scFv protein consisting essentially of the sequence set forth in any one of SEQ ID NOS: 494, 503, 505, and 508. Optionally, the anti-TfR scFv coding sequence in the above examples encodes an anti-TfR scFv protein consisting of the sequence set forth in any one of SEQ ID NOS: 494, 503, 505, and 508.

Various anti-TfR scFv coding sequences are provided. In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 530-532 and 536. In another example, the anti-TfR scFv coding sequence is (or comprises a sequence) at least 95%, at least 96%, at least 97%, at least 98%, at least 99%, at least 99.5%, or 100% identical to any one of SEQ ID NOS: 530-532 and 536. In another example, the anti-TfR scFv coding sequence is (or comprises a sequence) at least 99%, at least 99.5%, or 100% identical to any one of SEQ ID NOS: 530-532 and 536. In another example, the anti-TfR scFv coding sequence comprises the sequence set forth in any one of SEQ ID NOS: 530-532 and 536. In another example, the anti-TfR scFv coding sequence consists essentially of the sequence set forth in any one of SEQ ID NOS: 530-532 and 536. In another example, the anti-TfR scFv coding sequence consists of the sequence set forth in any one of SEQ ID NOS: 530-532 and 536. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 508 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 508 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 508 (and, e.g., retaining TfR-binding activity). Optionally, the anti-TfR scFv coding sequence in the above examples encodes an anti-TfR scFv protein comprising the sequence set forth in SEQ ID NO: 508. Optionally, the anti-TfR scFv coding sequence in the above examples encodes an anti-TfR scFv protein consisting essentially of the sequence set forth in SEQ ID NO: 508. Optionally, the anti-TfR scFv coding sequence in the above examples encodes an anti-TfR scFv protein consisting of the sequence set forth in SEQ ID NO: 508.

Various codon optimized anti-TfR scFv coding sequences are provided. The anti-TfR scFv coding sequence can be, for example, CpG-depleted (e.g., fully CpG depleted) and/or codon optimized (e.g., CpG depleted (e.g., fully CpG-depleted) and codon optimized). In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 524-532. In another example, the anti-TfR scFv coding sequence is (or comprises a sequence) at least 95%, at least 96%, at least 97%, at least 98%, at least 99%, at least 99.5%, or 100% identical to any one of SEQ ID NOS: 524-532. In another example, the anti-TfR scFv coding sequence is (or comprises a sequence) at least 99%, at least 99.5%, or 100% identical to any one of SEQ ID NOS: 524-532. In another example, the anti-TfR scFv coding sequence comprises the sequence set forth in any one of SEQ ID NOS: 524-532. In another example, the anti-TfR scFv coding sequence consists essentially of the sequence set forth in any one of SEQ ID NOS: 524-532. In another example, the anti-TfR scFv coding sequence consists of the sequence set forth in any one of SEQ ID NOS: 524-532. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 494, 503, 505, and 508 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to any one of SEQ ID NOS: 494, 503, 505, and 508 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to any one of SEQ ID NOS: 494, 503, 505, and 508 (and, e.g., retaining TfR-binding activity). Optionally, the anti-TfR scFv coding sequence in the above examples encodes an anti-TfR scFv protein comprising the sequence set forth in any one of SEQ ID NOS: 494, 503, 505, and 508. Optionally, the anti-TfR scFv coding sequence in the above examples encodes an anti-TfR scFv protein consisting essentially of the sequence set forth in any one of SEQ ID NOS: 494, 503, 505, and 508. Optionally, the anti-TfR scFv coding sequence in the above examples encodes an anti-TfR scFv protein consisting of the sequence set forth in any one of SEQ ID NOS: 494, 503, 505, and 508.

Various codon optimized anti-TfR scFv coding sequences are provided. The anti-TfR scFv coding sequence can be, for example, CpG-depleted (e.g., fully CpG depleted) and/or codon optimized (e.g., CpG depleted (e.g., fully CpG-depleted) and codon optimized). In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 530-532. In another example, the anti-TfR scFv coding sequence is (or comprises a sequence) at least 95%, at least 96%, at least 97%, at least 98%, at least 99%, at least 99.5%, or 100% identical to any one of SEQ ID NOS: 530-532. In another example, the anti-TfR scFv coding sequence is (or comprises a sequence) at least 99%, at least 99.5%, or 100% identical to any one of SEQ ID NOS: 530-532. In another example, the anti-TfR scFv coding sequence comprises the sequence set forth in any one of SEQ ID NOS: 530-532. In another example, the anti-TfR scFv coding sequence consists essentially of the sequence set forth in any one of SEQ ID NOS: 530-532. In another example, the anti-TfR scFv coding sequence consists of the sequence set forth in any one of SEQ ID NOS: 530-532. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 508 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 508 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 508 (and, e.g., retaining TfR-binding activity). Optionally, the anti-TfR scFv coding sequence in the above examples encodes an anti-TfR scFv protein comprising the sequence set forth in SEQ ID NO: 508. Optionally, the anti-TfR scFv coding sequence in the above examples encodes an anti-TfR scFv protein consisting essentially of the sequence set forth in SEQ ID NO: 508. Optionally, the anti-TfR scFv coding sequence in the above examples encodes an anti-TfR scFv protein consisting of the sequence set forth in SEQ ID NO: 508.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 530. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 530 and encodes an anti-TfR scFv protein (or an anti-TfR scFv protein comprising a sequence) at least 99%, at least 99.5%, or 100% identical to SEQ ID NO: 508. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 530 and encodes an anti-TfR scFv protein comprising the sequence set forth in SEQ ID NO: 508. In another example, the anti-TfR scFv coding sequence is (or comprises a sequence) at least 95%, at least 96%, at least 97%, at least 98%, at least 99%, at least 99.5%, or 100% identical to SEQ ID NO: 530. In another example, the anti-TfR scFv coding sequence is (or comprises a sequence) at least 95%, at least 96%, at least 97%, at least 98%, at least 99%, at least 99.5%, or 100% identical to SEQ ID NO: 530 and encodes an anti-TfR scFv protein (or an anti-TfR scFv protein comprising a sequence) at least 99%, at least 99.5%, or 100% identical to SEQ ID NO: 508. In another example, the anti-TfR scFv coding sequence is (or comprises a sequence) at least 95%, at least 96%, at least 97%, at least 98%, at least 99%, at least 99.5%, or 100% identical to SEQ ID NO: 530 and encodes an anti-TfR scFv protein comprising the sequence set forth in SEQ ID NO: 508. In another example, the anti-TfR scFv coding sequence is (or comprises a sequence) at least 99%, at least 99.5%, or 100% identical to SEQ ID NO: 530. In another example, the anti-TfR scFv coding sequence is (or comprises a sequence) at least 99%, at least 99.5%, or 100% identical to SEQ ID NO: 530 and encodes an anti-TfR scFv protein (or an anti-TfR scFv protein comprising a sequence) at least 99%, at least 99.5%, or 100% identical to SEQ ID NO: 508. In another example, the anti-TfR scFv coding sequence is (or comprises a sequence) at least 99%, at least 99.5%, or 100% identical to SEQ ID NO: 530 and encodes an anti-TfR scFv protein comprising the sequence set forth in SEQ ID NO: 508. In another example, the anti-TfR scFv coding sequence comprises the sequence set forth in SEQ ID NO: 530. In another example, the anti-TfR scFv coding sequence consists essentially of the sequence set forth in SEQ ID NO: 530. In another example, the anti-TfR scFv coding sequence consists of the sequence set forth in SEQ ID NO: 530. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 508 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 508 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 508 (and, e.g., retaining TfR-binding activity). Optionally, the anti-TfR scFv coding sequence in the above examples encodes an anti-TfR scFv protein comprising the sequence set forth in SEQ ID NO: 508. Optionally, the anti-TfR scFv coding sequence in the above examples encodes an anti-TfR scFv protein consisting essentially of the sequence set forth in SEQ ID NO: 508. Optionally, the anti-TfR scFv coding sequence in the above examples encodes an anti-TfR scFv protein consisting of the sequence set forth in SEQ ID NO: 508.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 531. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 531 and encodes an anti-TfR scFv protein (or an anti-TfR scFv protein comprising a sequence) at least 99%, at least 99.5%, or 100% identical to SEQ ID NO: 508. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 531 and encodes an anti-TfR scFv protein comprising the sequence set forth in SEQ ID NO: 508. In another example, the anti-TfR scFv coding sequence is (or comprises a sequence) at least 95%, at least 96%, at least 97%, at least 98%, at least 99%, at least 99.5%, or 100% identical to SEQ ID NO: 531. In another example, the anti-TfR scFv coding sequence is (or comprises a sequence) at least 95%, at least 96%, at least 97%, at least 98%, at least 99%, at least 99.5%, or 100% identical to SEQ ID NO: 531 and encodes an anti-TfR scFv protein (or an anti-TfR scFv protein comprising a sequence) at least 99%, at least 99.5%, or 100% identical to SEQ ID NO: 508. In another example, the anti-TfR scFv coding sequence is (or comprises a sequence) at least 95%, at least 96%, at least 97%, at least 98%, at least 99%, at least 99.5%, or 100% identical to SEQ ID NO: 531 and encodes an anti-TfR scFv protein comprising the sequence set forth in SEQ ID NO: 508. In another example, the anti-TfR scFv coding sequence is (or comprises a sequence) at least 99%, at least 99.5%, or 100% identical to SEQ ID NO: 531. In another example, the anti-TfR scFv coding sequence is (or comprises a sequence) at least 99%, at least 99.5%, or 100% identical to SEQ ID NO: 531 and encodes an anti-TfR scFv protein (or an anti-TfR scFv protein comprising a sequence) at least 99%, at least 99.5%, or 100% identical to SEQ ID NO: 508. In another example, the anti-TfR scFv coding sequence is (or comprises a sequence) at least 99%, at least 99.5%, or 100% identical to SEQ ID NO: 531 and encodes an anti-TfR scFv protein comprising the sequence set forth in SEQ ID NO: 508. In another example, the anti-TfR scFv coding sequence comprises the sequence set forth in SEQ ID NO: 531. In another example, the anti-TfR scFv coding sequence consists essentially of the sequence set forth in SEQ ID NO: 531. In another example, the anti-TfR scFv coding sequence consists of the sequence set forth in SEQ ID NO: 531. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 508 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 508 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 508 (and, e.g., retaining TfR-binding activity). Optionally, the anti-TfR scFv coding sequence in the above examples encodes an anti-TfR scFv protein comprising the sequence set forth in SEQ ID NO: 508. Optionally, the anti-TfR scFv coding sequence in the above examples encodes an anti-TfR scFv protein consisting essentially of the sequence set forth in SEQ ID NO: 508. Optionally, the anti-TfR scFv coding sequence in the above examples encodes an anti-TfR scFv protein consisting of the sequence set forth in SEQ ID NO: 508.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 532. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 532 and encodes an anti-TfR scFv protein (or an anti-TfR scFv protein comprising a sequence) at least 99%, at least 99.5%, or 100% identical to SEQ ID NO: 508. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 532 and encodes an anti-TfR scFv protein comprising the sequence set forth in SEQ ID NO: 508. In another example, the anti-TfR scFv coding sequence is (or comprises a sequence) at least 95%, at least 96%, at least 97%, at least 98%, at least 99%, at least 99.5%, or 100% identical to SEQ ID NO: 532. In another example, the anti-TfR scFv coding sequence is (or comprises a sequence) at least 95%, at least 96%, at least 97%, at least 98%, at least 99%, at least 99.5%, or 100% identical to SEQ ID NO: 532 and encodes an anti-TfR scFv protein (or an anti-TfR scFv protein comprising a sequence) at least 99%, at least 99.5%, or 100% identical to SEQ ID NO: 508. In another example, the anti-TfR scFv coding sequence is (or comprises a sequence) at least 95%, at least 96%, at least 97%, at least 98%, at least 99%, at least 99.5%, or 100% identical to SEQ ID NO: 532 and encodes an anti-TfR scFv protein comprising the sequence set forth in SEQ ID NO: 508. In another example, the anti-TfR scFv coding sequence is (or comprises a sequence) at least 99%, at least 99.5%, or 100% identical to SEQ ID NO: 532. In another example, the anti-TfR scFv coding sequence is (or comprises a sequence) at least 99%, at least 99.5%, or 100% identical to SEQ ID NO: 532 and encodes an anti-TfR scFv protein (or an anti-TfR scFv protein comprising a sequence) at least 99%, at least 99.5%, or 100% identical to SEQ ID NO: 508. In another example, the anti-TfR scFv coding sequence is (or comprises a sequence) at least 99%, at least 99.5%, or 100% identical to SEQ ID NO: 532 and encodes an anti-TfR scFv protein comprising the sequence set forth in SEQ ID NO: 508. In another example, the anti-TfR scFv coding sequence comprises the sequence set forth in SEQ ID NO: 532. In another example, the anti-TfR scFv coding sequence consists essentially of the sequence set forth in SEQ ID NO: 532. In another example, the anti-TfR scFv coding sequence consists of the sequence set forth in SEQ ID NO: 532. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 508 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 508 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 508 (and, e.g., retaining TfR-binding activity). Optionally, the anti-TfR scFv coding sequence in the above examples encodes an anti-TfR scFv protein comprising the sequence set forth in SEQ ID NO: 508. Optionally, the anti-TfR scFv coding sequence in the above examples encodes an anti-TfR scFv protein consisting essentially of the sequence set forth in SEQ ID NO: 508. Optionally, the anti-TfR scFv coding sequence in the above examples encodes an anti-TfR scFv protein consisting of the sequence set forth in SEQ ID NO: 508.

Various codon optimized anti-TfR scFv coding sequences are provided. The anti-TfR scFv coding sequence can be, for example, CpG-depleted (e.g., fully CpG depleted) and/or codon optimized (e.g., CpG depleted (e.g., fully CpG-depleted) and codon optimized). In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 527-529. In another example, the anti-TfR scFv coding sequence is (or comprises a sequence) at least 95%, at least 96%, at least 97%, at least 98%, at least 99%, at least 99.5%, or 100% identical to any one of SEQ ID NOS: 527-529. In another example, the anti-TfR scFv coding sequence is (or comprises a sequence) at least 99%, at least 99.5%, or 100% identical to any one of SEQ ID NOS: 527-529. In another example, the anti-TfR scFv coding sequence comprises the sequence set forth in any one of SEQ ID NOS: 527-529. In another example, the anti-TfR scFv coding sequence consists essentially of the sequence set forth in any one of SEQ ID NOS: 527-529. In another example, the anti-TfR scFv coding sequence consists of the sequence set forth in any one of SEQ ID NOS: 527-529. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 505 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 505 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 505 (and, e.g., retaining TfR-binding activity). Optionally, the anti-TfR scFv coding sequence in the above examples encodes an anti-TfR scFv protein comprising the sequence set forth in SEQ ID NO: 505. Optionally, the anti-TfR scFv coding sequence in the above examples encodes an anti-TfR scFv protein consisting essentially of the sequence set forth in SEQ ID NO: 505. Optionally, the anti-TfR scFv coding sequence in the above examples encodes an anti-TfR scFv protein consisting of the sequence set forth in SEQ ID NO: 505.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 527. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 527 and encodes an anti-TfR scFv protein (or an anti-TfR scFv protein comprising a sequence) at least 99%, at least 99.5%, or 100% identical to SEQ ID NO: 505. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 527 and encodes an anti-TfR scFv protein comprising the sequence set forth in SEQ ID NO: 505. In another example, the anti-TfR scFv coding sequence is (or comprises a sequence) at least 95%, at least 96%, at least 97%, at least 98%, at least 99%, at least 99.5%, or 100% identical to SEQ ID NO: 527. In another example, the anti-TfR scFv coding sequence is (or comprises a sequence) at least 95%, at least 96%, at least 97%, at least 98%, at least 99%, at least 99.5%, or 100% identical to SEQ ID NO: 527 and encodes an anti-TfR scFv protein (or an anti-TfR scFv protein comprising a sequence) at least 99%, at least 99.5%, or 100% identical to SEQ ID NO: 505. In another example, the anti-TfR scFv coding sequence is (or comprises a sequence) at least 95%, at least 96%, at least 97%, at least 98%, at least 99%, at least 99.5%, or 100% identical to SEQ ID NO: 527 and encodes an anti-TfR scFv protein comprising the sequence set forth in SEQ ID NO: 505. In another example, the anti-TfR scFv coding sequence is (or comprises a sequence) at least 99%, at least 99.5%, or 100% identical to SEQ ID NO: 527. In another example, the anti-TfR scFv coding sequence is (or comprises a sequence) at least 99%, at least 99.5%, or 100% identical to SEQ ID NO: 527 and encodes an anti-TfR scFv protein (or an anti-TfR scFv protein comprising a sequence) at least 99%, at least 99.5%, or 100% identical to SEQ ID NO: 505. In another example, the anti-TfR scFv coding sequence is (or comprises a sequence) at least 99%, at least 99.5%, or 100% identical to SEQ ID NO: 527 and encodes an anti-TfR scFv protein comprising the sequence set forth in SEQ ID NO: 505. In another example, the anti-TfR scFv coding sequence comprises the sequence set forth in SEQ ID NO: 527. In another example, the anti-TfR scFv coding sequence consists essentially of the sequence set forth in SEQ ID NO: 527. In another example, the anti-TfR scFv coding sequence consists of the sequence set forth in SEQ ID NO: 527. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 505 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 505 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 505 (and, e.g., retaining TfR-binding activity). Optionally, the anti-TfR scFv coding sequence in the above examples encodes an anti-TfR scFv protein comprising the sequence set forth in SEQ ID NO: 505. Optionally, the anti-TfR scFv coding sequence in the above examples encodes an anti-TfR scFv protein consisting essentially of the sequence set forth in SEQ ID NO: 505. Optionally, the anti-TfR scFv coding sequence in the above examples encodes an anti-TfR scFv protein consisting of the sequence set forth in SEQ ID NO: 505.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 528. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 528 and encodes an anti-TfR scFv protein (or an anti-TfR scFv protein comprising a sequence) at least 99%, at least 99.5%, or 100% identical to SEQ ID NO: 505. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 528 and encodes an anti-TfR scFv protein comprising the sequence set forth in SEQ ID NO: 505. In another example, the anti-TfR scFv coding sequence is (or comprises a sequence) at least 95%, at least 96%, at least 97%, at least 98%, at least 99%, at least 99.5%, or 100% identical to SEQ ID NO: 528. In another example, the anti-TfR scFv coding sequence is (or comprises a sequence) at least 95%, at least 96%, at least 97%, at least 98%, at least 99%, at least 99.5%, or 100% identical to SEQ ID NO: 528 and encodes an anti-TfR scFv protein (or an anti-TfR scFv protein comprising a sequence) at least 99%, at least 99.5%, or 100% identical to SEQ ID NO: 505. In another example, the anti-TfR scFv coding sequence is (or comprises a sequence) at least 95%, at least 96%, at least 97%, at least 98%, at least 99%, at least 99.5%, or 100% identical to SEQ ID NO: 528 and encodes an anti-TfR scFv protein comprising the sequence set forth in SEQ ID NO: 505. In another example, the anti-TfR scFv coding sequence is (or comprises a sequence) at least 99%, at least 99.5%, or 100% identical to SEQ ID NO: 528. In another example, the anti-TfR scFv coding sequence is (or comprises a sequence) at least 99%, at least 99.5%, or 100% identical to SEQ ID NO: 528 and encodes an anti-TfR scFv protein (or an anti-TfR scFv protein comprising a sequence) at least 99%, at least 99.5%, or 100% identical to SEQ ID NO: 505. In another example, the anti-TfR scFv coding sequence is (or comprises a sequence) at least 99%, at least 99.5%, or 100% identical to SEQ ID NO: 528 and encodes an anti-TfR scFv protein comprising the sequence set forth in SEQ ID NO: 505. In another example, the anti-TfR scFv coding sequence comprises the sequence set forth in SEQ ID NO: 528. In another example, the anti-TfR scFv coding sequence consists essentially of the sequence set forth in SEQ ID NO: 528. In another example, the anti-TfR scFv coding sequence consists of the sequence set forth in SEQ ID NO: 528. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 505 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 505 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 505 (and, e.g., retaining TfR-binding activity). Optionally, the anti-TfR scFv coding sequence in the above examples encodes an anti-TfR scFv protein comprising the sequence set forth in SEQ ID NO: 505. Optionally, the anti-TfR scFv coding sequence in the above examples encodes an anti-TfR scFv protein consisting essentially of the sequence set forth in SEQ ID NO: 505. Optionally, the anti-TfR scFv coding sequence in the above examples encodes an anti-TfR scFv protein consisting of the sequence set forth in SEQ ID NO: 505.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 529. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 529 and encodes an anti-TfR scFv protein (or an anti-TfR scFv protein comprising a sequence) at least 99%, at least 99.5%, or 100% identical to SEQ ID NO: 505. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 529 and encodes an anti-TfR scFv protein comprising the sequence set forth in SEQ ID NO: 505. In another example, the anti-TfR scFv coding sequence is (or comprises a sequence) at least 95%, at least 96%, at least 97%, at least 98%, at least 99%, at least 99.5%, or 100% identical to SEQ ID NO: 529. In another example, the anti-TfR scFv coding sequence is (or comprises a sequence) at least 95%, at least 96%, at least 97%, at least 98%, at least 99%, at least 99.5%, or 100% identical to SEQ ID NO: 529 and encodes an anti-TfR scFv protein (or an anti-TfR scFv protein comprising a sequence) at least 99%, at least 99.5%, or 100% identical to SEQ ID NO: 505. In another example, the anti-TfR scFv coding sequence is (or comprises a sequence) at least 95%, at least 96%, at least 97%, at least 98%, at least 99%, at least 99.5%, or 100% identical to SEQ ID NO: 529 and encodes an anti-TfR scFv protein comprising the sequence set forth in SEQ ID NO: 505. In another example, the anti-TfR scFv coding sequence is (or comprises a sequence) at least 99%, at least 99.5%, or 100% identical to SEQ ID NO: 529. In another example, the anti-TfR scFv coding sequence is (or comprises a sequence) at least 99%, at least 99.5%, or 100% identical to SEQ ID NO: 529 and encodes an anti-TfR scFv protein (or an anti-TfR scFv protein comprising a sequence) at least 99%, at least 99.5%, or 100% identical to SEQ ID NO: 505. In another example, the anti-TfR scFv coding sequence is (or comprises a sequence) at least 99%, at least 99.5%, or 100% identical to SEQ ID NO: 529 and encodes an anti-TfR scFv protein comprising the sequence set forth in SEQ ID NO: 505. In another example, the anti-TfR scFv coding sequence comprises the sequence set forth in SEQ ID NO: 529. In another example, the anti-TfR scFv coding sequence consists essentially of the sequence set forth in SEQ ID NO: 529. In another example, the anti-TfR scFv coding sequence consists of the sequence set forth in SEQ ID NO: 529. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 505 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 505 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 505 (and, e.g., retaining TfR-binding activity). Optionally, the anti-TfR scFv coding sequence in the above examples encodes an anti-TfR scFv protein comprising the sequence set forth in SEQ ID NO: 505. Optionally, the anti-TfR scFv coding sequence in the above examples encodes an anti-TfR scFv protein consisting essentially of the sequence set forth in SEQ ID NO: 505. Optionally, the anti-TfR scFv coding sequence in the above examples encodes an anti-TfR scFv protein consisting of the sequence set forth in SEQ ID NO: 505.

Various codon optimized anti-TfR scFv coding sequences are provided. The anti-TfR scFv coding sequence can be, for example, CpG-depleted (e.g., fully CpG depleted) and/or codon optimized (e.g., CpG depleted (e.g., fully CpG-depleted) and codon optimized). In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 524-526. In another example, the anti-TfR scFv coding sequence is (or comprises a sequence) at least 95%, at least 96%, at least 97%, at least 98%, at least 99%, at least 99.5%, or 100% identical to any one of SEQ ID NOS: 524-526. In another example, the anti-TfR scFv coding sequence is (or comprises a sequence) at least 99%, at least 99.5%, or 100% identical to any one of SEQ ID NOS: 524-526. In another example, the anti-TfR scFv coding sequence comprises the sequence set forth in any one of SEQ ID NOS: 524-526. In another example, the anti-TfR scFv coding sequence consists essentially of the sequence set forth in any one of SEQ ID NOS: 524-526. In another example, the anti-TfR scFv coding sequence consists of the sequence set forth in any one of SEQ ID NOS: 524-526. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 494 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 494 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 494 (and, e.g., retaining TfR-binding activity). Optionally, the anti-TfR scFv coding sequence in the above examples encodes an anti-TfR scFv protein comprising the sequence set forth in SEQ ID NO: 494. Optionally, the anti-TfR scFv coding sequence in the above examples encodes an anti-TfR scFv protein consisting essentially of the sequence set forth in SEQ ID NO: 494. Optionally, the anti-TfR scFv coding sequence in the above examples encodes an anti-TfR scFv protein consisting of the sequence set forth in SEQ ID NO: 494.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 524. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 524 and encodes an anti-TfR scFv protein (or an anti-TfR scFv protein comprising a sequence) at least 99%, at least 99.5%, or 100% identical to SEQ ID NO: 494. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 524 and encodes an anti-TfR scFv protein comprising the sequence set forth in SEQ ID NO: 494. In another example, the anti-TfR scFv coding sequence is (or comprises a sequence) at least 95%, at least 96%, at least 97%, at least 98%, at least 99%, at least 99.5%, or 100% identical to SEQ ID NO: 524. In another example, the anti-TfR scFv coding sequence is (or comprises a sequence) at least 95%, at least 96%, at least 97%, at least 98%, at least 99%, at least 99.5%, or 100% identical to SEQ ID NO: 524 and encodes an anti-TfR scFv protein (or an anti-TfR scFv protein comprising a sequence) at least 99%, at least 99.5%, or 100% identical to SEQ ID NO: 494. In another example, the anti-TfR scFv coding sequence is (or comprises a sequence) at least 95%, at least 96%, at least 97%, at least 98%, at least 99%, at least 99.5%, or 100% identical to SEQ ID NO: 524 and encodes an anti-TfR scFv protein comprising the sequence set forth in SEQ ID NO: 494. In another example, the anti-TfR scFv coding sequence is (or comprises a sequence) at least 99%, at least 99.5%, or 100% identical to SEQ ID NO: 524. In another example, the anti-TfR scFv coding sequence is (or comprises a sequence) at least 99%, at least 99.5%, or 100% identical to SEQ ID NO: 524 and encodes an anti-TfR scFv protein (or an anti-TfR scFv protein comprising a sequence) at least 99%, at least 99.5%, or 100% identical to SEQ ID NO: 494. In another example, the anti-TfR scFv coding sequence is (or comprises a sequence) at least 99%, at least 99.5%, or 100% identical to SEQ ID NO: 524 and encodes an anti-TfR scFv protein comprising the sequence set forth in SEQ ID NO: 494. In another example, the anti-TfR scFv coding sequence comprises the sequence set forth in SEQ ID NO: 524. In another example, the anti-TfR scFv coding sequence consists essentially of the sequence set forth in SEQ ID NO: 524. In another example, the anti-TfR scFv coding sequence consists of the sequence set forth in SEQ ID NO: 524. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 494 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 494 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 494 (and, e.g., retaining TfR-binding activity). Optionally, the anti-TfR scFv coding sequence in the above examples encodes an anti-TfR scFv protein comprising the sequence set forth in SEQ ID NO: 494. Optionally, the anti-TfR scFv coding sequence in the above examples encodes an anti-TfR scFv protein consisting essentially of the sequence set forth in SEQ ID NO: 494. Optionally, the anti-TfR scFv coding sequence in the above examples encodes an anti-TfR scFv protein consisting of the sequence set forth in SEQ ID NO: 494.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 525. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 525 and encodes an anti-TfR scFv protein (or an anti-TfR scFv protein comprising a sequence) at least 99%, at least 99.5%, or 100% identical to SEQ ID NO: 494. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 525 and encodes an anti-TfR scFv protein comprising the sequence set forth in SEQ ID NO: 494. In another example, the anti-TfR scFv coding sequence is (or comprises a sequence) at least 95%, at least 96%, at least 97%, at least 98%, at least 99%, at least 99.5%, or 100% identical to SEQ ID NO: 525. In another example, the anti-TfR scFv coding sequence is (or comprises a sequence) at least 95%, at least 96%, at least 97%, at least 98%, at least 99%, at least 99.5%, or 100% identical to SEQ ID NO: 525 and encodes an anti-TfR scFv protein (or an anti-TfR scFv protein comprising a sequence) at least 99%, at least 99.5%, or 100% identical to SEQ ID NO: 494. In another example, the anti-TfR scFv coding sequence is (or comprises a sequence) at least 95%, at least 96%, at least 97%, at least 98%, at least 99%, at least 99.5%, or 100% identical to SEQ ID NO: 525 and encodes an anti-TfR scFv protein comprising the sequence set forth in SEQ ID NO: 494. In another example, the anti-TfR scFv coding sequence is (or comprises a sequence) at least 99%, at least 99.5%, or 100% identical to SEQ ID NO: 525. In another example, the anti-TfR scFv coding sequence is (or comprises a sequence) at least 99%, at least 99.5%, or 100% identical to SEQ ID NO: 525 and encodes an anti-TfR scFv protein (or an anti-TfR scFv protein comprising a sequence) at least 99%, at least 99.5%, or 100% identical to SEQ ID NO: 494. In another example, the anti-TfR scFv coding sequence is (or comprises a sequence) at least 99%, at least 99.5%, or 100% identical to SEQ ID NO: 525 and encodes an anti-TfR scFv protein comprising the sequence set forth in SEQ ID NO: 494. In another example, the anti-TfR scFv coding sequence comprises the sequence set forth in SEQ ID NO: 525. In another example, the anti-TfR scFv coding sequence consists essentially of the sequence set forth in SEQ ID NO: 525. In another example, the anti-TfR scFv coding sequence consists of the sequence set forth in SEQ ID NO: 525. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 494 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 494 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 494 (and, e.g., retaining TfR-binding activity). Optionally, the anti-TfR scFv coding sequence in the above examples encodes an anti-TfR scFv protein comprising the sequence set forth in SEQ ID NO: 494. Optionally, the anti-TfR scFv coding sequence in the above examples encodes an anti-TfR scFv protein consisting essentially of the sequence set forth in SEQ ID NO: 494. Optionally, the anti-TfR scFv coding sequence in the above examples encodes an anti-TfR scFv protein consisting of the sequence set forth in SEQ ID NO: 494.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 526. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 526 and encodes an anti-TfR scFv protein (or an anti-TfR scFv protein comprising a sequence) at least 99%, at least 99.5%, or 100% identical to SEQ ID NO: 494. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 526 and encodes an anti-TfR scFv protein comprising the sequence set forth in SEQ ID NO: 494. In another example, the anti-TfR scFv coding sequence is (or comprises a sequence) at least 95%, at least 96%, at least 97%, at least 98%, at least 99%, at least 99.5%, or 100% identical to SEQ ID NO: 526. In another example, the anti-TfR scFv coding sequence is (or comprises a sequence) at least 95%, at least 96%, at least 97%, at least 98%, at least 99%, at least 99.5%, or 100% identical to SEQ ID NO: 526 and encodes an anti-TfR scFv protein (or an anti-TfR scFv protein comprising a sequence) at least 99%, at least 99.5%, or 100% identical to SEQ ID NO: 494. In another example, the anti-TfR scFv coding sequence is (or comprises a sequence) at least 95%, at least 96%, at least 97%, at least 98%, at least 99%, at least 99.5%, or 100% identical to SEQ ID NO: 526 and encodes an anti-TfR scFv protein comprising the sequence set forth in SEQ ID NO: 494. In another example, the anti-TfR scFv coding sequence is (or comprises a sequence) at least 99%, at least 99.5%, or 100% identical to SEQ ID NO: 526. In another example, the anti-TfR scFv coding sequence is (or comprises a sequence) at least 99%, at least 99.5%, or 100% identical to SEQ ID NO: 526 and encodes an anti-TfR scFv protein (or an anti-TfR scFv protein comprising a sequence) at least 99%, at least 99.5%, or 100% identical to SEQ ID NO: 494. In another example, the anti-TfR scFv coding sequence is (or comprises a sequence) at least 99%, at least 99.5%, or 100% identical to SEQ ID NO: 526 and encodes an anti-TfR scFv protein comprising the sequence set forth in SEQ ID NO: 494. In another example, the anti-TfR scFv coding sequence comprises the sequence set forth in SEQ ID NO: 526. In another example, the anti-TfR scFv coding sequence consists essentially of the sequence set forth in SEQ ID NO: 526. In another example, the anti-TfR scFv coding sequence consists of the sequence set forth in SEQ ID NO: 526. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 494 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 494 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 494 (and, e.g., retaining TfR-binding activity). Optionally, the anti-TfR scFv coding sequence in the above examples encodes an anti-TfR scFv protein comprising the sequence set forth in SEQ ID NO: 494. Optionally, the anti-TfR scFv coding sequence in the above examples encodes an anti-TfR scFv protein consisting essentially of the sequence set forth in SEQ ID NO: 494. Optionally, the anti-TfR scFv coding sequence in the above examples encodes an anti-TfR scFv protein consisting of the sequence set forth in SEQ ID NO: 494.

When specific anti-TfR scFv or anti-TfR Fab or multidomain therapeutic protein nucleic acid constructs sequences are disclosed herein, they are meant to encompass the sequence disclosed or the reverse complement of the sequence. For example, if an anti-TfR scFv or anti-TfR Fab or multidomain therapeutic protein nucleic acid construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the anti-TfR scFv or anti-TfR Fab or multidomain therapeutic protein nucleic acid constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### (2) Bidirectional Constructs

The nucleic acid constructs disclosed herein can be bidirectional constructs. Such bidirectional constructs can allow for enhanced insertion and expression of encoded multidomain therapeutic protein. When used in combination with a nuclease agent (e.g., CRISPR/Cas system, zinc finger nuclease (ZFN) system; transcription activator-like effector nuclease (TALEN) system) as described herein, the bidirectionality of the nucleic acid construct allows the construct to be inserted in either direction (i.e., is not limited to insertion in one direction) within a target genomic locus or a cleavage site or target insertion site, allowing the expression of the multidomain therapeutic protein when inserted in either orientation, thereby enhancing expression efficiency.

A bidirectional construct as disclosed herein can comprise at least two nucleic acid segments, wherein a first segment comprises a first coding sequence for the multidomain therapeutic protein, and a second segment comprises the reverse complement of a second coding sequence for the multidomain therapeutic protein, or vice versa. However, other bidirectional constructs disclosed herein can comprise at least two nucleic acid segments, wherein the first segment comprises a coding sequence for a multidomain therapeutic protein, and the second segment comprises the reverse complement of a coding sequence for another protein, or vice versa. A reverse complement refers to a sequence that is a complement sequence of a reference sequence, wherein the complement sequence is written in the reverse orientation. For example, for a hypothetical sequence 5′-CTGGACCGA-3′, the perfect complement sequence is 3′-GACCTGGCT-5′, and the perfect reverse complement is written 5′-TCGGTCCAG-3′. A reverse complement sequence need not be perfect and may still encode the same polypeptide or a similar polypeptide as the reference sequence. Due to codon usage redundancy, a reverse complement can diverge from a reference sequence that encodes the same polypeptide. The coding sequences can optionally comprise one or more additional sequences, such as sequences encoding amino- or carboxy-terminal amino acid sequences such as a signal sequence, label sequence (e.g., HiBit), or heterologous functional sequence (e.g., nuclear localization sequence (NLS) or self-cleaving peptide) linked to the multidomain therapeutic protein or other protein.

When specific bidirectional construct sequences are disclosed herein, they are meant to encompass the sequence disclosed or the reverse complement of the sequence. For example, if a bidirectional construct disclosed herein consists of the hypothetical sequence 5′-CTGGACCGA-3′, it is also meant to encompass the reverse complement of that sequence (5′-TCGGTCCAG-3′). Likewise, when bidirectional construct elements are disclosed herein in a specific 5′ to 3′ order, they are also meant to encompass the reverse complement of the order of those elements. For example, if a bidirectional construct is disclosed herein that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. One reason for this is that, in many embodiments disclosed herein, the bidirectional constructs are part of a single-stranded recombinant AAV vector. Single-stranded AAV genomes are packaged as either sense (plus-stranded) or anti-sense (minus-stranded genomes), and single-stranded AAV genomes of + and -polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

When the at least two segments both encode a multidomain therapeutic protein, the at least two segments can encode the same multidomain therapeutic protein or multidomain therapeutic protein. The different multidomain therapeutic proteins can be at least about 90%, at least about 95%, at least about 96%, at least about 97%, at least about 98%, at least about 99%, or at least about 99.5% identical. Preferably, the two segments encode the same multidomain therapeutic protein (i.e., 100% identical).

Even when the two segments encode the same multidomain therapeutic protein, the coding sequence for the multidomain therapeutic protein in the first segment can differ from the coding sequence for the multidomain therapeutic protein in the second segment. In some bidirectional constructs, the codon usage in the first coding sequence is the same as the codon usage in the second coding sequence. In other bidirectional constructs, the second coding sequence adopts a different codon usage from the codon usage of the first coding sequence in order to reduce hairpin formation. One or both of the coding sequences can be codon-optimized for expression in a host cell. In some bidirectional constructs, only one of the coding sequences is codon-optimized. In some bidirectional constructs, the first coding sequence is codon-optimized. In some bidirectional constructs, the second coding sequence is codon-optimized. In some bidirectional constructs, both coding sequences are codon-optimized. For example, the second multidomain therapeutic protein coding sequence can be codon optimized or may use one or more alternative codons for one or more amino acids of the same multidomain therapeutic protein (i.e., same amino acid sequence) encoded by the multidomain therapeutic protein coding sequence in the first segment. An alternative codon as used herein refers to variations in codon usage for a given amino acid, and may or may not be a preferred or optimized codon (codon optimized) for a given expression system. Preferred codon usage, or codons that are well-tolerated in a given system of expression are known.

In one example, the second segment comprises a reverse complement of a multidomain therapeutic protein coding sequence that adopts different codon usage from that of the multidomain therapeutic protein coding sequence in the first segment in order to reduce hairpin formation. Such a reverse complement forms base pairs with fewer than all nucleotides of the coding sequence in the first segment, yet it optionally encodes the same polypeptide. In one example, the reverse complement sequence in the second segment is not substantially complementary (e.g., not more than 70% complementary) to the coding sequence in the first segment. In other cases, however, the second segment comprises a reverse complement sequence that is highly complementary (e.g., at least 90% complementary) to the coding sequence in the first segment.

The second segment can have any percentage of complementarity to the first segment. For example, the second segment sequence can have at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, or at least about 99% complementarity to the first segment. As another example, the second segment sequence can have less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, less than about 95%, less than about 97%, or less than about 99% complementarity to the first segment. The reverse complement of the second coding sequence can be, in some nucleic acid constructs, not substantially complementary (e.g., not more than 70% complementary) to the first coding sequence, not substantially complementary to a fragment of the first coding sequence, highly complementary (e.g., at least 90% complementary) to the first coding sequence, highly complementary to a fragment of the first coding sequence, about 50% to about 80% identical to the reverse complement of the first coding sequence, or about 60% to about 100% identical to the reverse complement of the first coding sequence.

The bidirectional constructs disclosed herein can be modified to include any suitable structural feature as needed for any particular use and/or that confers one or more desired function. For example, the bidirectional nucleic acid constructs disclosed herein need not comprise a homology arm and/or can be, for example, homology-independent donor constructs. Owing in part to the bidirectional function of the nucleic acid constructs, the bidirectional constructs can be inserted into a genomic locus in either direction as described herein to allow for efficient insertion and/or expression of the multidomain therapeutic protein.

In some cases, the bidirectional nucleic acid construct does not comprise a promoter that drives the expression of the multidomain therapeutic protein. For example, the expression of the multidomain therapeutic protein can be driven by a promoter of the host cell (e.g., the endogenous ALB promoter when the transgene is integrated into a host cell's ALB locus). In other cases, the bidirectional nucleic acid construct can comprise one or more promoters operably linked to the coding sequences for the multidomain therapeutic protein. That is, although not required for expression, the constructs disclosed herein may also include transcriptional or translational regulatory sequences such as promoters, enhancers, insulators, internal ribosome entry sites, additional sequences encoding peptides, and/or polyadenylation signals. Some bidirectional constructs can comprise a promoter that drives expression of the first multidomain therapeutic protein coding sequence and/or the reverse complement of a promoter that drives expression of the reverse complement of the second multidomain therapeutic protein coding sequence.

The bidirectional constructs disclosed herein can be modified to include or exclude any suitable structural feature as needed for any particular use and/or that confers one or more desired functions. For example, some bidirectional nucleic acid constructs disclosed herein do not comprise a homology arm. Owing in part to the bidirectional function of the nucleic acid construct, the bidirectional construct can be inserted into a genomic locus in either direction (orientation) as described herein to allow for efficient insertion and/or expression of a multidomain therapeutic protein.

The bidirectional constructs can, in some cases, comprise one or more (e.g., two) polyadenylation tail sequences or polyadenylation signal sequences. In some bidirectional constructs, the first segment can comprise a polyadenylation signal sequence. In some bidirectional constructs, the second segment can comprise a polyadenylation signal sequence. In some bidirectional constructs, the first segment can comprise a first polyadenylation signal sequence, and the second segment can comprise a second polyadenylation signal sequence (e.g., a reverse complement of a polyadenylation signal sequence). In some bidirectional constructs, the first segment can comprise a first polyadenylation signal sequence located 3′ of the first coding sequence. In some bidirectional constructs, the second segment can comprise a reverse complement of a second polyadenylation signal sequence located 5′ of the reverse complement of the second coding sequence. In some bidirectional constructs, the first segment can comprise a first polyadenylation signal sequence located 3′ of the first coding sequence, and the second segment can comprise a reverse complement of a second polyadenylation signal sequence located 5′ of the reverse complement of the second coding sequence. The first and second polyadenylation signal sequences can be the same or different. In one example, the first and second polyadenylation signals are different. In a specific example, the first polyadenylation signal is a simian virus 40 (SV40) late polyadenylation signal (or a variant thereof), and the second polyadenylation signal is a bovine growth hormone (BGH) polyadenylation signal (or a variant thereof), or vice versa. For example, one polyadenylation signal can be an SV40 polyadenylation signal, and the other polyadenylation signal can be a BGH polyadenylation signal. In a specific example, one polyadenylation signal can comprise, consist essentially of, or consist of SEQ ID NO: 161, and the other polyadenylation signal can comprise, consist essentially of, or consist of SEQ ID NO: 162.

In one example, either polyadenylation signal can comprise a BGH polyadenylation signal. For example, the BGH polyadenylation signal can comprise, consist essentially of, or consist of SEQ ID NO: 751. In another example, the polyadenylation signal can comprise an SV40 polyadenylation signal. For example, the SV40 polyadenylation signal can be a unidirectional SV40 late polyadenylation signal. For example, the transcription terminator sequences that are present in the “early” inverse orientation of SV40 can be mutated (e.g., by mutating the reverse strand AAUAAA sequences to AAUCAA). The SV40 polyA is bidirectional, but the polyadenylation in the “late” orientation is more efficient than the polyadenylation in the “early” orientation. For example, the unidirectional SV40 late polyadenylation signal can comprise, consist essentially of, or consist of SEQ ID NO: 752. In another example, a synthetic polyadenylation signal can be used. For example, the synthetic polyadenylation signal can comprise, consist essentially of, or consist of SEQ ID NO: 753. In another example, two or more polyadenylation signals can be used in combination. For example, the polyadenylation signal can comprise a combination of a BGH polyadenylation signal and an SV40 polyadenylation signal (e.g., an SV40 late polyadenylation signal, such as a unidirectional SV40 late polyadenylation signal). For example, the polyadenylation signal can comprise a combination of a BGH polyadenylation signal and a unidirectional SV40 late polyadenylation signal. For example, the BGH polyadenylation signal can comprise, consist essentially of, or consist of SEQ ID NO: 751, and the unidirectional SV40 late polyadenylation signal can comprise, consist essentially of, or consist of SEQ ID NO: 752. In a specific example, the BGH polyadenylation signal can be upstream (5′) of the SV40 polyadenylation signal (e.g., unidirectional SV40 late polyadenylation signal). For example, the combined polyadenylation signal can comprise the sequence set forth in SEQ ID NO: 795. In another example, the polyadenylation signal can comprise a combination of a BGH polyadenylation signal and a synthetic polyadenylation signal. For example, the BGH polyadenylation signal can comprise, consist essentially of, or consist of SEQ ID NO: 751, and the synthetic polyadenylation signal can comprise, consist essentially of, or consist of SEQ ID NO: 753.

In some embodiments, a stuffer sequence can be used to increase the time between when RNA polymerase transcribes the polyA to the time when it transcribes the next splice acceptor. For example, the stuffer sequence can be used between two different polyadenylation signals (e.g., between a BGH polyadenylation signal and a synthetic polyadenylation signal. For example, the stuffer sequence can comprise, consist essentially of, or consist of SEQ ID NO: 754.

In some embodiments, MAZ elements that cause polymerase pausing are used in combination with a polyadenylation signal (e.g., a BGH polyadenylation signal or an SV40 polyadenylation signal). For example, one or more (e.g., at least 1, at least 2, at least 3, at least 4, or about 1 to about 4, about 2 to about 4, about 3 to about 4, or 1, 2, 3, or 4) MAZ elements can be used in combination with a polyadenylation signal. For example, the MAZ element can comprise, consist essentially of, or consist of SEQ ID NO: 755.

In some embodiments, unidirectional SV40 late polyadenylation signals are used. The SV40 polyA is bidirectional, but the polyadenylation in the “late” orientation is more efficient than the polyadenylation in the “early” orientation. The unidirectional SV40 late polyadenylation signals described herein are positioned in the “late” orientation, with the polyadenylation signals present in the “early” orientation mutated or inactivated. In some embodiments, each instance of the sequence AATAAA in the reverse strand is mutated in the unidirectional SV40 late polyadenylation signal. For example, the two conserved AATAAA poly(A) signals present in the SV40 “early” poly(A) to AATCAA. In some embodiments, the unidirectional SV40 late polyadenylation signal is at least 95%, at least 96%, at least 97%, at least 98%, or at least 99% identical to the sequence set forth in SEQ ID NO: 752. In some embodiments, the unidirectional SV40 late polyadenylation signal comprises, consists essentially of, or consists of the sequence set forth in SEQ ID NO: 752.

The unidirectional SV40 late polyadenylation signals can be used in combination with (e.g., in tandem with) one or more additional polyadenylation signals. Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. For example, the unidirectional SV40 late polyadenylation signals can be used in combination with (e.g., in tandem with) a bovine growth hormone (BGH) polyadenylation signal, optionally wherein the BGH polyadenylation signal is upstream of (5′ of) the unidirectional SV40 late polyadenylation signal. In some embodiments, the BGH polyadenylation signal is at least 95%, at least 96%, at least 97%, at least 98%, or at least 99% identical to the sequence set forth in SEQ ID NO: 751. In some embodiments, the BGH polyadenylation signal comprises, consists essentially of, or consists of the sequence set forth in SEQ ID NO: 751. In some embodiments, the combination of the BGH polyadenylation signal and the unidirectional SV40 late polyadenylation signal is at least 95%, at least 96%, at least 97%, at least 98%, or at least 99% identical to the sequence set forth in SEQ ID NO: 795. In some embodiments, the combination of the BGH polyadenylation signal and the unidirectional SV40 late polyadenylation signal comprises, consists essentially of, or consists of the sequence set forth in SEQ ID NO: 795.

In some embodiments, a stuffer sequence can be used to increase the time between when RNA polymerase transcribes the polyA to the time when it transcribes the next splice acceptor. For example, the stuffer sequence can be used between two different polyadenylation signals (e.g., between a BGH polyadenylation signal and a synthetic polyadenylation signal. For example, the stuffer sequence can comprise, consist essentially of, or consist of SEQ ID NO: 754.

In some embodiments, MAZ elements that cause polymerase pausing are used in combination with a polyadenylation signal (e.g., a BGH polyadenylation signal or an SV40 polyadenylation signal). For example, one or more (e.g., at least 1, at least 2, at least 3, at least 4, or about 1 to about 4, about 2 to about 4, about 3 to about 4, or 1, 2, 3, or 4) MAZ elements can be used in combination with a polyadenylation signal. For example, the MAZ element can comprise, consist essentially of, or consist of SEQ ID NO: 755.

In some bidirectional constructs, both the first segment and the second segment comprise a polyadenylation tail sequence. Methods of designing a suitable polyadenylation tail sequence are known. For example, in some bidirectional constructs, one or both of the first and second segment comprises a polyadenylation tail sequence and/or a polyadenylation signal sequence downstream of an open reading frame (i.e., a polyadenylation tail sequence and/or a polyadenylation signal sequence 3′ of a coding sequence, or a reverse complement of a polyadenylation tail sequence and/or a polyadenylation signal sequence 5′ of a reverse complement of a coding sequence). The polyadenylation tail sequence can be encoded, for example, as a “poly-A” stretch downstream of the multidomain therapeutic protein coding sequence (or other protein coding sequence) in the first and/or second segment. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes. In some bidirectional constructs, a single bidirectional terminator can be used to terminate RNA polymerase transcription in either the sense or the antisense direction (i.e., to terminate RNA polymerase transcription from both the first segment and the second segment). Examples of bidirectional terminators include the ARO4, TRP1, TRP4, ADH1, CYC1, GAL1, GAL7, and GAL10 terminators.

The bidirectional constructs can, in some cases, comprise one or more (e.g., two) splice acceptor sites. In some bidirectional constructs, the first segment can comprise a splice acceptor site. In some bidirectional constructs, the second segment can comprise a splice acceptor site. In some bidirectional constructs, the first segment can comprise a first splice acceptor site, and the second segment can comprise a second splice acceptor site (e.g., a reverse complement of a splice acceptor site). In some bidirectional constructs, the first segment comprises a first splice acceptor site located 5′ of the first coding sequence. In some bidirectional constructs, the second segment comprises a reverse complement of a second splice acceptor site located 3′ of the reverse complement of the second coding sequence. In some bidirectional constructs, the first segment comprises a first splice acceptor site located 5′ of the first coding sequence, and the second segment comprises a reverse complement of a second splice acceptor site located 3′ of the reverse complement of the second coding sequence. The first and second splice acceptor sites can be the same or different. In a specific example, both splice acceptors are mouse Alb exon 2 splice acceptors. In a specific example, both splice acceptors can comprise, consist essentially of, or consist of SEQ ID NO: 163.

A bidirectional construct may comprise a first coding sequence that encodes a first coding sequence linked to a splice acceptor and a reverse complement of a second coding sequence operably linked to the reverse complement of a splice acceptor. The bidirectional constructs disclosed herein can also comprise a splice acceptor site on either or both ends of the construct, or splice acceptor sites in both the first segment and the second segment (e.g., a splice acceptor site 5′ of a coding sequence, or a reverse complement of a splice acceptor 3′ of a reverse complement of a coding sequence). The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a splice acceptor from a gene encoding the multidomain therapeutic protein. Additional suitable splice acceptor sites useful in eukaryotes, including artificial splice acceptors, are known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes. The splice acceptors used in a bidirectional construct may be the same or different. In a specific example, both splice acceptors are mouse Alb exon 2 splice acceptors.

The bidirectional constructs can be circular or linear. For example, a bidirectional construct can be linear. The first and second segments can be joined in a linear manner through a linker sequence. For example, the 5′ end of the second segment that comprises a reverse complement sequence can be linked to the 3′ end of the first segment. Alternatively, the 5′ end of the first segment can be linked to the 3′ end of the second segment that comprises a reverse complement sequence. The linker can be any suitable length. For example, the linker can be between about 5 to about 2000 nucleotides in length. As an example, the linker sequence can be about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 150, about 200, about 250, about 300, about 500, about 1000, about 1500, about 2000, or more nucleotides in length. Other structural elements in addition to, or instead of, a linker sequence, can also be inserted between the first and second segments.

The bidirectional constructs disclosed herein can be DNA or RNA, single-stranded, double-stranded, or partially single-stranded and partially double-stranded. For example, the constructs can be single- or double-stranded DNA. In some embodiments, the nucleic acid can be modified (e.g., using nucleoside analogs), as described herein. In a specific example, the bidirectional construct is single-stranded (e.g., single-stranded DNA).

The bidirectional constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Similarly, one or both ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

As disclosed in more detail herein, the bidirectional constructs disclosed herein can be introduced into a cell as part of a vector having additional sequences such as, for example, replication origins, promoters, and genes encoding antibiotic resistance. The constructs can be introduced as a naked nucleic acid, can be introduced as a nucleic acid complexed with an agent such as a liposome, polymer, or poloxamer, or can be delivered by viral vectors (e.g., adenovirus, AAV, herpesvirus, retrovirus, lentivirus).

In an exemplary bidirectional construct, the second segment is located 3′ of the first segment, the first multidomain therapeutic protein coding sequence and the second multidomain therapeutic protein coding sequence both encode the same multidomain therapeutic protein, the second multidomain therapeutic protein coding sequence adopts a different codon usage from the codon usage of the first multidomain therapeutic protein coding sequence, the first segment comprises a first polyadenylation signal sequence located 3′ of the first multidomain therapeutic protein coding sequence, the second segment comprises a reverse complement of a second polyadenylation signal sequence located 5′ of the reverse complement of the second multidomain therapeutic protein coding sequence, the first segment comprises a first splice acceptor site located 5′ of the first multidomain therapeutic protein coding sequence, the second segment comprises a reverse complement of a second splice acceptor site located 3′ of the reverse complement of the second multidomain therapeutic protein coding sequence, the nucleic acid construct does not comprise a promoter that drives expression of the first multidomain therapeutic protein or the second multidomain therapeutic protein, and optionally the nucleic acid construct does not comprise a homology arm.

### (3) Unidirectional Constructs

The nucleic acid constructs disclosed herein can be unidirectional constructs. When specific unidirectional construct sequences are disclosed herein, they are meant to encompass the sequence disclosed or the reverse complement of the sequence. For example, if a unidirectional construct disclosed herein consists of the hypothetical sequence 5′-CTGGACCGA-3′, it is also meant to encompass the reverse complement of that sequence (5′-TCGGTCCAG-3′). Likewise, when unidirectional construct elements are disclosed herein in a specific 5′ to 3′ order, they are also meant to encompass the reverse complement of the order of those elements. One reason for this is that, in many embodiments disclosed herein, the unidirectional constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

In the unidirectional constructs, the coding sequence for the multidomain therapeutic protein can be codon-optimized for expression in a host cell. For example, the coding sequence can be codon optimized or may use one or more alternative codons for one or more amino acids of the multidomain therapeutic protein (i.e., same amino acid sequence). An alternative codon as used herein refers to variations in codon usage for a given amino acid, and may or may not be a preferred or optimized codon (codon optimized) for a given expression system. Preferred codon usage, or codons that are well-tolerated in a given system of expression, are known.

The unidirectional constructs disclosed herein can be modified to include any suitable structural feature as needed for any particular use and/or that confers one or more desired functions. For example, the unidirectional nucleic acid constructs disclosed herein need not comprise a homology arm and/or can be, for example, homology-independent donor constructs.

In some cases, the unidirectional nucleic acid construct does not comprise a promoter that drives the expression of multidomain therapeutic protein. For example, the expression of the multidomain therapeutic protein can be driven by a promoter of the host cell (e.g., the endogenous ALB promoter when the transgene is integrated into a host cell's ALB locus). In other cases, the unidirectional nucleic acid construct can comprise one or more promoters operably linked to the coding sequence for the multidomain therapeutic protein. That is, although not required for expression, the constructs disclosed herein may also include transcriptional or translational regulatory sequences such as promoters, enhancers, insulators, internal ribosome entry sites, additional sequences encoding peptides, and/or polyadenylation signals. Some unidirectional constructs can comprise a promoter that drives expression of the coding sequence for the multidomain therapeutic protein.

The unidirectional constructs can, in some cases, comprise one or more polyadenylation tail sequences or polyadenylation signal sequences. Some unidirectional constructs can comprise a polyadenylation signal sequence located 3′ of the coding sequence for the multidomain therapeutic protein. In a specific example, the polyadenylation signal is a simian virus 40 (SV40) late polyadenylation signal (or a variant thereof). In another specific example, the polyadenylation signal is a bovine growth hormone (BGH) polyadenylation signal (or a variant thereof). In another specific example, the polyadenylation signal is a BGH polyadenylation signal. For example, the polyadenylation signal can be an SV40 polyadenylation signal or a BGH polyadenylation signal. In a specific example, the polyadenylation signal can comprise, consist essentially of, or consist of SEQ ID NO: 161. In another specific example, the polyadenylation signal can comprise, consist essentially of, or consist of SEQ ID NO: 162.

In one example, the polyadenylation signal can comprise a BGH polyadenylation signal. For example, the BGH polyadenylation signal can comprise, consist essentially of, or consist of SEQ ID NO: 751. In another example, the polyadenylation signal can comprise an SV40 polyadenylation signal. For example, the SV40 polyadenylation signal can be a unidirectional SV40 late polyadenylation signal. For example, the transcription terminator sequences that are present in the “early” inverse orientation of SV40 can be mutated (e.g., by mutating the reverse strand AAUAAA sequences to AAUCAA). The SV40 polyA is bidirectional, but the polyadenylation in the “late” orientation is more efficient than the polyadenylation in the “early” orientation. For example, the unidirectional SV40 late polyadenylation signal can comprise, consist essentially of, or consist of SEQ ID NO: 752. In another example, a synthetic polyadenylation signal can be used. For example, the synthetic polyadenylation signal can comprise, consist essentially of, or consist of SEQ ID NO: 753. In another example, two or more polyadenylation signals can be used in combination. For example, the polyadenylation signal can comprise a combination of a BGH polyadenylation signal and an SV40 polyadenylation signal (e.g., an SV40 late polyadenylation signal, such as a unidirectional SV40 late polyadenylation signal). For example, the polyadenylation signal can comprise a combination of a BGH polyadenylation signal and a unidirectional SV40 late polyadenylation signal. For example, the BGH polyadenylation signal can comprise, consist essentially of, or consist of SEQ ID NO: 751, and the unidirectional SV40 late polyadenylation signal can comprise, consist essentially of, or consist of SEQ ID NO: 752. In a specific example, the BGH polyadenylation signal can be upstream (5′) of the SV40 polyadenylation signal (e.g., unidirectional SV40 late polyadenylation signal). For example, the combined polyadenylation signal can comprise the sequence set forth in SEQ ID NO: 795. In another example, the polyadenylation signal can comprise a combination of a BGH polyadenylation signal and a synthetic polyadenylation signal. For example, the BGH polyadenylation signal can comprise, consist essentially of, or consist of SEQ ID NO: 751, and the synthetic polyadenylation signal can comprise, consist essentially of, or consist of SEQ ID NO: 753.

In some embodiments, a stuffer sequence can be used to increase the time between when RNA polymerase transcribes the polyA to the time when it transcribes the next splice acceptor. For example, the stuffer sequence can be used between two different polyadenylation signals (e.g., between a BGH polyadenylation signal and a synthetic polyadenylation signal. For example, the stuffer sequence can comprise, consist essentially of, or consist of SEQ ID NO: 754.

In some embodiments, MAZ elements that cause polymerase pausing are used in combination with a polyadenylation signal (e.g., a BGH polyadenylation signal or an SV40 polyadenylation signal). For example, one or more (e.g., at least 1, at least 2, at least 3, at least 4, or about 1 to about 4, about 2 to about 4, about 3 to about 4, or 1, 2, 3, or 4) MAZ elements can be used in combination with a polyadenylation signal. For example, the MAZ element can comprise, consist essentially of, or consist of SEQ ID NO: 755.

In some embodiments, unidirectional SV40 late polyadenylation signals are used. The SV40 polyA is bidirectional, but the polyadenylation in the “late” orientation is more efficient than the polyadenylation in the “early” orientation. The unidirectional SV40 late polyadenylation signals described herein are positioned in the “late” orientation, with the polyadenylation signals present in the “early” orientation mutated or inactivated. In some embodiments, each instance of the sequence AATAAA in the reverse strand is mutated in the unidirectional SV40 late polyadenylation signal. For example, the two conserved AATAAA poly(A) signals present in the SV40 “early” poly(A) to AATCAA. In some embodiments, the unidirectional SV40 late polyadenylation signal is at least 95%, at least 96%, at least 97%, at least 98%, or at least 99% identical to the sequence set forth in SEQ ID NO: 752. In some embodiments, the unidirectional SV40 late polyadenylation signal comprises, consists essentially of, or consists of the sequence set forth in SEQ ID NO: 752.

The unidirectional SV40 late polyadenylation signals can be used in combination with (e.g., in tandem with) one or more additional polyadenylation signals. Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. For example, the unidirectional SV40 late polyadenylation signals can be used in combination with (e.g., in tandem with) a bovine growth hormone (BGH) polyadenylation signal, optionally wherein the BGH polyadenylation signal is upstream of (5′ of) the unidirectional SV40 late polyadenylation signal. In some embodiments, the BGH polyadenylation signal is at least 95%, at least 96%, at least 97%, at least 98%, or at least 99% identical to the sequence set forth in SEQ ID NO: 751. In some embodiments, the BGH polyadenylation signal comprises, consists essentially of, or consists of the sequence set forth in SEQ ID NO: 751. In some embodiments, the combination of the BGH polyadenylation signal and the unidirectional SV40 late polyadenylation signal is at least 95%, at least 96%, at least 97%, at least 98%, or at least 99% identical to the sequence set forth in SEQ ID NO: 795. In some embodiments, the combination of the BGH polyadenylation signal and the unidirectional SV40 late polyadenylation signal comprises, consists essentially of, or consists of the sequence set forth in SEQ ID NO: 795.

In some embodiments, a stuffer sequence can be used to increase the time between when RNA polymerase transcribes the polyA to the time when it transcribes the next splice acceptor. For example, the stuffer sequence can be used between two different polyadenylation signals (e.g., between a BGH polyadenylation signal and a synthetic polyadenylation signal. For example, the stuffer sequence can comprise, consist essentially of, or consist of SEQ ID NO: 754.

In some embodiments, MAZ elements that cause polymerase pausing are used in combination with a polyadenylation signal (e.g., a BGH polyadenylation signal or an SV40 polyadenylation signal). For example, one or more (e.g., at least 1, at least 2, at least 3, at least 4, or about 1 to about 4, about 2 to about 4, about 3 to about 4, or 1, 2, 3, or 4) MAZ elements can be used in combination with a polyadenylation signal. For example, the MAZ element can comprise, consist essentially of, or consist of SEQ ID NO: 755.

Methods of designing a suitable polyadenylation tail sequence are known. For example, some unidirectional constructs comprise a polyadenylation tail sequence and/or a polyadenylation signal sequence downstream of an open reading frame (i.e., a polyadenylation tail sequence and/or a polyadenylation signal sequence 3′ of a coding sequence). The polyadenylation tail sequence can be encoded, for example, as a “poly-A” stretch downstream of the coding sequence for the multidomain therapeutic protein (or other protein coding sequence) in the first and/or second segment. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes.

The unidirectional constructs can, in some cases, comprise one or more splice acceptor sites. Some unidirectional constructs comprise a splice acceptor site located 5′ of the coding sequence for the multidomain therapeutic protein. In a specific example, the splice acceptor is a mouse Alb exon 2 splice acceptor. In a specific example, the splice acceptor can comprise, consist essentially of, or consist of SEQ ID NO: 163.

The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a splice acceptor from the gene encoding the multidomain therapeutic protein. Additional suitable splice acceptor sites useful in eukaryotes, including artificial splice acceptors, are known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes.

The unidirectional constructs can be circular or linear. For example, a unidirectional construct can be linear.

The unidirectional constructs disclosed herein can be DNA or RNA, single-stranded, double-stranded, or partially single-stranded and partially double-stranded. For example, the constructs can be single- or double-stranded DNA. In some embodiments, the nucleic acid can be modified (e.g., using nucleoside analogs), as described herein. In a specific example, the unidirectional construct is single-stranded (e.g., single-stranded DNA).

The unidirectional constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three JTRs or can comprise no more than two JTRs. Various methods of structural modifications are known.

Similarly, one or both ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

As disclosed in more detail herein, the unidirectional constructs disclosed herein can be introduced into a cell as part of a vector having additional sequences such as, for example, replication origins, promoters, and genes encoding antibiotic resistance. The constructs can be introduced as a naked nucleic acid, can be introduced as a nucleic acid complexed with an agent such as a liposome, polymer, or poloxamer, or can be delivered by viral vectors (e.g., adenovirus, AAV, herpesvirus, retrovirus, lentivirus).

In an exemplary unidirectional construct, the construct comprises a polyadenylation signal sequence located 3′ of the coding sequence for the multidomain therapeutic protein, the construct comprises a splice acceptor site located 5′ of the coding sequence for the multidomain therapeutic protein, and the nucleic acid construct does not comprise a promoter that drives expression of the multidomain therapeutic protein, and optionally the nucleic acid construct does not comprise a homology arm.

### (4) Multidomain Therapeutic Protein Nucleic Acid Constructs

The multidomain therapeutic protein nucleic acid constructs disclosed herein can be unidirectional constructs or bidirectional constructs. When specific construct sequences are disclosed herein, they are meant to encompass the sequence disclosed or the reverse complement of the sequence. For example, if a construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

In the nucleic acid constructs, the multidomain therapeutic protein coding sequence, the TfR-binding delivery domain or CD63-binding delivery domain coding sequence, and/or the GAA coding sequence can be codon-optimized for expression in a host cell. For example, the multidomain therapeutic protein coding sequence, the TfR-binding delivery domain or CD63-binding delivery domain coding sequence, and/or the GAA coding sequence can be codon optimized or may use one or more alternative codons for one or more amino acids of the protein (i.e., same amino acid sequence). An alternative codon as used herein refers to variations in codon usage for a given amino acid, and may or may not be a preferred or optimized codon (codon optimized) for a given expression system. Preferred codon usage, or codons that are well-tolerated in a given system of expression, are known.

The nucleic acid constructs disclosed herein can be modified to include any suitable structural feature as needed for any particular use and/or that confers one or more desired functions. For example, the nucleic acid constructs disclosed herein need not comprise a homology arm and/or can be, for example, homology-independent donor constructs.

In some cases, the nucleic acid construct does not comprise a promoter that drives the expression of the multidomain therapeutic protein. For example, the expression of the multidomain therapeutic protein can be driven by a promoter of the host cell (e.g., the endogenous ALB promoter when the transgene is integrated into a host cell's ALB locus). In other cases, the nucleic acid construct can comprise one or more promoters operably linked to the multidomain therapeutic protein coding sequence. That is, although not required for expression, the constructs disclosed herein may also include transcriptional or translational regulatory sequences such as promoters, enhancers, insulators, internal ribosome entry sites, additional sequences encoding peptides, and/or polyadenylation signals. Some nucleic acid constructs can comprise a promoter that drives expression of the multidomain therapeutic protein. For example, the promoter may be a liver-specific promoter. Examples of liver-specific promoters include TTR promoters, such as human or mouse TTR promoters. In one example, the construct may comprise a TTR promoter, such as a mouse TTR promoter or a human TTR promoter (e.g., the coding sequence for the multidomain therapeutic protein is operably linked to the TTR promoter). In one example, the construct may comprise a SERPINA1 enhancer, such as a mouse SERPINA1 enhancer or a human SERPINA1 enhancer (e.g., the coding sequence for the multidomain therapeutic protein is operably linked to the SERPINA1 enhancer). In one example, the construct may comprise a TTR promoter and a SERPINA1 enhancer, such as a human SERPINA1 enhancer and a mouse TTR promoter (e.g., the coding sequence for the multidomain therapeutic protein is operably linked to the SERPINA1 enhancer and the TTR promoter).

The nucleic acid constructs can, in some cases, comprise one or more polyadenylation tail sequences or polyadenylation signal sequences. Some nucleic acid constructs can comprise a polyadenylation signal sequence located 3′ of the multidomain therapeutic protein coding sequence. In a specific example, the polyadenylation signal is a simian virus 40 (SV40) late polyadenylation signal (or a variant thereof). In another specific example, the polyadenylation signal is a bovine growth hormone (BGH) polyadenylation signal (or a variant thereof). In another specific example, the polyadenylation signal is a CpG-depleted BGH polyadenylation signal. For example, the polyadenylation signal can be an SV40 polyadenylation signal or a CpG-depleted BGH polyadenylation signal. For example, the polyadenylation signal can comprise, consist essentially of, or consist of SEQ ID NO: 615, 169, or 161. In a specific example, the polyadenylation signal can comprise, consist essentially of, or consist of SEQ ID NO: 615. In a specific example, the polyadenylation signal can comprise, consist essentially of, or consist of SEQ ID NO: 169. In another specific example, the polyadenylation signal can comprise, consist essentially of, or consist of SEQ ID NO: 161. In another specific example, the polyadenylation signal can comprise, consist essentially of, or consist of SEQ ID NO: 162.

In one example, the polyadenylation signal can comprise a BGH polyadenylation signal. For example, the BGH polyadenylation signal can comprise, consist essentially of, or consist of SEQ ID NO: 751. In another example, the polyadenylation signal can comprise an SV40 polyadenylation signal. For example, the SV40 polyadenylation signal can be a unidirectional SV40 late polyadenylation signal. For example, the transcription terminator sequences that are present in the “early” inverse orientation of SV40 can be mutated (e.g., by mutating the reverse strand AAUAAA sequences to AAUCAA). The SV40 polyA is bidirectional, but the polyadenylation in the “late” orientation is more efficient than the polyadenylation in the “early” orientation. For example, the unidirectional SV40 late polyadenylation signal can comprise, consist essentially of, or consist of SEQ ID NO: 752. In another example, a synthetic polyadenylation signal can be used. For example, the synthetic polyadenylation signal can comprise, consist essentially of, or consist of SEQ ID NO: 753. In another example, two or more polyadenylation signals can be used in combination. For example, the polyadenylation signal can comprise a combination of a BGH polyadenylation signal and an SV40 polyadenylation signal (e.g., an SV40 late polyadenylation signal, such as a unidirectional SV40 late polyadenylation signal). For example, the polyadenylation signal can comprise a combination of a BGH polyadenylation signal and a unidirectional SV40 late polyadenylation signal. For example, the BGH polyadenylation signal can comprise, consist essentially of, or consist of SEQ ID NO: 751, and the unidirectional SV40 late polyadenylation signal can comprise, consist essentially of, or consist of SEQ ID NO: 752. In a specific example, the BGH polyadenylation signal can be upstream (5′) of the SV40 polyadenylation signal (e.g., unidirectional SV40 late polyadenylation signal). For example, the combined polyadenylation signal can comprise the sequence set forth in SEQ ID NO: 795. In another example, the polyadenylation signal can comprise a combination of a BGH polyadenylation signal and a synthetic polyadenylation signal. For example, the BGH polyadenylation signal can comprise, consist essentially of, or consist of SEQ ID NO: 751, and the synthetic polyadenylation signal can comprise, consist essentially of, or consist of SEQ ID NO: 753. In some embodiments, the nucleic acid construct is a unidirectional construct.

In some embodiments, a stuffer sequence can be used to increase the time between when RNA polymerase transcribes the polyA to the time when it transcribes the next splice acceptor. For example, the stuffer sequence can be used between two different polyadenylation signals (e.g., between a BGH polyadenylation signal and a synthetic polyadenylation signal. For example, the stuffer sequence can comprise, consist essentially of, or consist of SEQ ID NO: 754.

In some embodiments, MAZ elements that cause polymerase pausing are used in combination with a polyadenylation signal (e.g., a BGH polyadenylation signal or an SV40 polyadenylation signal). For example, one or more (e.g., at least 1, at least 2, at least 3, at least 4, or about 1 to about 4, about 2 to about 4, about 3 to about 4, or 1, 2, 3, or 4) MAZ elements can be used in combination with a polyadenylation signal. For example, the MAZ element can comprise, consist essentially of, or consist of SEQ ID NO: 755.

In some embodiments, unidirectional SV40 late polyadenylation signals are used. The SV40 polyA is bidirectional, but the polyadenylation in the “late” orientation is more efficient than the polyadenylation in the “early” orientation. The unidirectional SV40 late polyadenylation signals described herein are positioned in the “late” orientation, with the polyadenylation signals present in the “early” orientation mutated or inactivated. In some embodiments, each instance of the sequence AATAAA in the reverse strand is mutated in the unidirectional SV40 late polyadenylation signal. For example, the two conserved AATAAA poly(A) signals present in the SV40 “early” poly(A) to AATCAA. In some embodiments, the unidirectional SV40 late polyadenylation signal is at least 95%, at least 96%, at least 97%, at least 98%, or at least 99% identical to the sequence set forth in SEQ ID NO: 752. In some embodiments, the unidirectional SV40 late polyadenylation signal comprises, consists essentially of, or consists of the sequence set forth in SEQ ID NO: 752.

The unidirectional SV40 late polyadenylation signals can be used in combination with (e.g., in tandem with) one or more additional polyadenylation signals. Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. For example, the unidirectional SV40 late polyadenylation signals can be used in combination with (e.g., in tandem with) a bovine growth hormone (BGH) polyadenylation signal, optionally wherein the BGH polyadenylation signal is upstream of (5′ of) the unidirectional SV40 late polyadenylation signal. In some embodiments, the BGH polyadenylation signal is at least 95%, at least 96%, at least 97%, at least 98%, or at least 99% identical to the sequence set forth in SEQ ID NO: 751. In some embodiments, the BGH polyadenylation signal comprises, consists essentially of, or consists of the sequence set forth in SEQ ID NO: 751. In some embodiments, the combination of the BGH polyadenylation signal and the unidirectional SV40 late polyadenylation signal is at least 95%, at least 96%, at least 97%, at least 98%, or at least 99% identical to the sequence set forth in SEQ ID NO: 795. In some embodiments, the combination of the BGH polyadenylation signal and the unidirectional SV40 late polyadenylation signal comprises, consists essentially of, or consists of the sequence set forth in SEQ ID NO: 795.

In some embodiments, a stuffer sequence can be used to increase the time between when RNA polymerase transcribes the polyA to the time when it transcribes the next splice acceptor. For example, the stuffer sequence can be used between two different polyadenylation signals (e.g., between a BGH polyadenylation signal and a synthetic polyadenylation signal. For example, the stuffer sequence can comprise, consist essentially of, or consist of SEQ ID NO: 754.

In some embodiments, MAZ elements that cause polymerase pausing are used in combination with a polyadenylation signal (e.g., a BGH polyadenylation signal or an SV40 polyadenylation signal). For example, one or more (e.g., at least 1, at least 2, at least 3, at least 4, or about 1 to about 4, about 2 to about 4, about 3 to about 4, or 1, 2, 3, or 4) MAZ elements can be used in combination with a polyadenylation signal. For example, the MAZ element can comprise, consist essentially of, or consist of SEQ ID NO: 755.

Methods of designing a suitable polyadenylation tail sequence are known. For example, some nucleic acid constructs comprise a polyadenylation tail sequence and/or a polyadenylation signal sequence downstream of an open reading frame (i.e., a polyadenylation tail sequence and/or a polyadenylation signal sequence 3′ of a coding sequence). The polyadenylation tail sequence can be encoded, for example, as a “poly-A” stretch downstream of the multidomain therapeutic protein coding sequence (or other protein coding sequence) in the first and/or second segment. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes.

The nucleic acid constructs can, in some cases, comprise one or more splice acceptor sites. Some nucleic acid constructs comprise a splice acceptor site located 5′ of the multidomain therapeutic protein coding sequence. In a specific example, the splice acceptor is a mouse Alb exon 2 splice acceptor. In a specific example, the splice acceptor can comprise, consist essentially of, or consist of SEQ ID NO: 163.

The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a GAA splice acceptor. For example, such a splice acceptor can be derived from the human GAA gene. Alternatively, such a splice acceptor can be derived from the mouse GAA gene. Additional suitable splice acceptor sites useful in eukaryotes, including artificial splice acceptors, are known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes.

The nucleic acid constructs can be circular or linear. For example, a nucleic acid construct can be linear. The nucleic acid constructs disclosed herein can be DNA or RNA, single-stranded, double-stranded, or partially single-stranded and partially double-stranded. For example, the constructs can be single- or double-stranded DNA. In some embodiments, the nucleic acid can be modified (e.g., using nucleoside analogs), as described herein. In a specific example, the nucleic acid construct is single-stranded (e.g., single-stranded DNA).

The nucleic acid constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the nucleic acid constructs disclosed herein can comprise one, two, or three ITRs or can comprise no more than two ITRs. Various methods of structural modifications are known.

Similarly, one or both ends of the nucleic acid construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

As disclosed in more detail herein, the nucleic acid constructs disclosed herein can be introduced into a cell as part of a vector having additional sequences such as, for example, replication origins, promoters, and genes encoding antibiotic resistance. The nucleic acid constructs can be introduced as a naked nucleic acid, can be introduced as a nucleic acid complexed with an agent such as a liposome, polymer, or poloxamer, or can be delivered by viral vectors (e.g., adenovirus, AAV, herpesvirus, retrovirus, lentivirus).

The multidomain therapeutic protein coding sequence, the TfR-binding delivery domain or CD63-binding delivery domain coding sequence, and/or the GAA coding sequence in the nucleic acid constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a multidomain therapeutic protein coding sequence, a TfR-binding delivery domain or CD63-binding delivery domain coding sequence, and/or a GAA coding sequence in a nucleic acid construct disclosed herein has one or more cryptic splice sites mutated or removed. In another example, a multidomain therapeutic protein coding sequence, a TfR-binding delivery domain or CD63-binding delivery domain coding sequence, and/or a GAA coding sequence in a nucleic acid construct disclosed herein has all identified cryptic splice sites mutated or removed. In another example, a multidomain therapeutic protein coding sequence, a TfR-binding delivery domain or CD63-binding delivery domain coding sequence, and/or a GAA coding sequence in a nucleic acid construct disclosed herein has one or more CpG dinucleotides removed (i.e., is CpG depleted). In another example, a multidomain therapeutic protein coding sequence, a TfR-binding delivery domain or CD63-binding delivery domain coding sequence, and/or a GAA coding sequence in a nucleic acid construct disclosed herein has all CpG dinucleotides removed. In another example, a multidomain therapeutic protein coding sequence, a TfR-binding delivery domain or CD63-binding delivery domain coding sequence, and/or a GAA coding sequence in a nucleic acid construct disclosed herein is codon optimized (e.g., codon optimized for expression in a human or mammal). In a specific example, a multidomain therapeutic protein coding sequence, a TfR-binding delivery domain or CD63-binding delivery domain coding sequence, and/or a GAA coding sequence in a nucleic acid construct disclosed herein has one or more CpG dinucleotides removed (i.e., is CpG depleted) and has one or more cryptic splice sites mutated or removed. In another specific example, a multidomain therapeutic protein coding sequence, a TfR-binding delivery domain or CD63-binding delivery domain coding sequence, and/or a GAA coding sequence in a nucleic acid construct disclosed herein has all CpG dinucleotides removed and has one or more or all identified cryptic splice sites mutated or removed. In another specific example, a multidomain therapeutic protein coding sequence, a TfR-binding delivery domain or CD63-binding delivery domain coding sequence, and/or a GAA coding sequence in a nucleic acid construct disclosed herein has one or more CpG dinucleotides removed (i.e., is CpG depleted) and is codon optimized (e.g., codon optimized for expression in a human or mammal). In another specific example, a multidomain therapeutic protein coding sequence, a TfR-binding delivery domain or CD63-binding delivery domain coding sequence, and/or a GAA coding sequence in a nucleic acid construct disclosed herein has all CpG dinucleotides removed (i.e., is fully CpG depleted) and is codon optimized (e.g., codon optimized for expression in a human or mammal).

In an exemplary nucleic acid construct, the construct comprises a polyadenylation signal sequence located 3′ of the multidomain therapeutic protein coding sequence, the construct comprises a splice acceptor site located 5′ of the multidomain therapeutic protein coding sequence, and the nucleic acid construct comprises a promoter that drives expression of the multidomain therapeutic protein.

In an exemplary nucleic acid construct, the construct comprises a polyadenylation signal sequence located 3′ of the multidomain therapeutic protein coding sequence, and the nucleic acid construct comprises a promoter that drives expression of the multidomain therapeutic protein (e.g., for episomal gene expression).

In an exemplary nucleic acid construct, the construct comprises a polyadenylation signal sequence located 3′ of the multidomain therapeutic protein coding sequence, the construct comprises a splice acceptor site located 5′ of the multidomain therapeutic protein coding sequence, and the nucleic acid construct does not comprise a promoter that drives expression of the multidomain therapeutic protein, and optionally the nucleic acid construct does not comprise a homology arm.

In a specific example of a multidomain therapeutic protein nucleic acid construct, the encoded multidomain therapeutic protein can comprise SEQ ID NO: 746 or can be at least 90%, at least 91%, at least 92%, at least 93%, at least 94%, at least 95%, at least 96%, at least 97%, at least 98%, at least 99%, or at least 99.5% identical to SEQ ID NO: 746. In another specific example, the multidomain therapeutic protein can consist essentially of SEQ ID NO: 746. In another specific example, the multidomain therapeutic protein can consist of SEQ ID NO: 746.

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 745 or 671. In another example, the multidomain therapeutic protein coding sequence is (or comprises a sequence) at least 95%, at least 96%, at least 97%, at least 98%, at least 99%, at least 99.5%, or 100% identical to SEQ ID NO: 745 or 671. In another example, the multidomain therapeutic protein coding sequence is (or comprises a sequence) at least 99%, at least 99.5%, or 100% identical to SEQ ID NO: 745 or 671. In another example, the multidomain therapeutic protein coding sequence comprises the sequence set forth in SEQ ID NO: 745 or 671. In another example, the multidomain therapeutic protein coding sequence consists essentially of the sequence set forth in SEQ ID NO: 745 or 671. In another example, the multidomain therapeutic protein coding sequence consists of the sequence set forth in SEQ ID NO: 745 or 671. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 746 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 746 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 746 (and, e.g., retaining the activity of native GAA). Optionally, the multidomain therapeutic protein coding sequence in the above examples encodes a multidomain therapeutic protein comprising the sequence set forth in SEQ ID NO: 746. Optionally, the multidomain therapeutic protein coding sequence in the above examples encodes a multidomain therapeutic protein consisting essentially of the sequence set forth in SEQ ID NO: 746. Optionally, the multidomain therapeutic protein coding sequence in the above examples encodes a multidomain therapeutic protein consisting of the sequence set forth in SEQ ID NO: 746. In some embodiments, the nucleotide at position 1857 is a “G.” In some embodiments, the nucleotide at position 1860 is a “C.” In some embodiments, the nucleotide at position 3105 is a “G.” In some embodiments, the nucleotide at position 1857 is a “G,” the nucleotide at position 1860 is a “C,” and the nucleotide at position 3105 is a “G.”

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 745. In another example, the multidomain therapeutic protein coding sequence is (or comprises a sequence) at least 95%, at least 96%, at least 97%, at least 98%, at least 99%, at least 99.5%, or 100% identical to SEQ ID NO: 745. In another example, the multidomain therapeutic protein coding sequence is (or comprises a sequence) at least 99%, at least 99.5%, or 100% identical to SEQ ID NO: 745. In another example, the multidomain therapeutic protein coding sequence comprises the sequence set forth in SEQ ID NO: 745. In another example, the multidomain therapeutic protein coding sequence consists essentially of the sequence set forth in SEQ ID NO: 745. In another example, the multidomain therapeutic protein coding sequence consists of the sequence set forth in SEQ ID NO: 745. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 746 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 746 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 746 (and, e.g., retaining the activity of native GAA). Optionally, the multidomain therapeutic protein coding sequence in the above examples encodes a multidomain therapeutic protein comprising the sequence set forth in SEQ ID NO: 746. Optionally, the multidomain therapeutic protein coding sequence in the above examples encodes a multidomain therapeutic protein consisting essentially of the sequence set forth in SEQ ID NO: 746. Optionally, the multidomain therapeutic protein coding sequence in the above examples encodes a multidomain therapeutic protein consisting of the sequence set forth in SEQ ID NO: 746. In some embodiments, the nucleotide at position 1857 is a “G.” In some embodiments, the nucleotide at position 1860 is a “C.” In some embodiments, the nucleotide at position 3105 is a “G.” In some embodiments, the nucleotide at position 1857 is a “G,” the nucleotide at position 1860 is a “C,” and the nucleotide at position 3105 is a “G.”

The nucleic acid construct can comprise, for example, (1) a 5′ ITR (e.g., such as the one set forth in SEQ ID NO: 160), (2) a splice acceptor site (e.g., a mouse Alb exon 2 splice acceptor, such as the one set forth in SEQ ID NO: 163), (3) the multidomain therapeutic protein coding sequence, (4) a polyadenylation signal (e.g., a BGH polyadenylation signal, such as the one set forth in SEQ ID NO: 751, or a combination of a BGH polyadenylation signal and a unidirectional SV40 late polyadenylation signal, such as the ones set forth in SEQ ID NOS: 751 and 752, respectively), and (5) a 3′ ITR (e.g., such as the one set forth in SEQ ID NO: 160 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 764, 765, 780, or 781. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 764, 765, 780, or 781 and encodes a multidomain therapeutic protein (or a multidomain therapeutic protein comprising a sequence) at least 99%, at least 99.5%, or 100% identical to SEQ ID NO: 746. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 764, 765, 780, or 781 and encodes a multidomain therapeutic protein comprising the sequence set forth in SEQ ID NO: 746. In another example, the nucleic acid construct comprises a sequence at least 95%, at least 96%, at least 97%, at least 98%, at least 99%, at least 99.5%, or 100% identical to SEQ ID NO: 764, 765, 780, or 781. In another example, the nucleic acid construct comprises a sequence at least 95%, at least 96%, at least 97%, at least 98%, at least 99%, at least 99.5%, or 100% identical to SEQ ID NO: 764, 765, 780, or 781 and encodes a multidomain therapeutic protein (or a multidomain therapeutic protein comprising a sequence) at least 99%, at least 99.5%, or 100% identical to SEQ ID NO: 746. In another example, the nucleic acid construct comprises a sequence at least 95%, at least 96%, at least 97%, at least 98%, at least 99%, at least 99.5%, or 100% identical to SEQ ID NO: 764, 765, 780, or 781 and encodes a multidomain therapeutic protein comprising the sequence set forth in SEQ ID NO: 746. In another example, the nucleic acid construct comprises a sequence at least 99%, at least 99.5%, or 100% identical to SEQ ID NO: 764, 765, 780, or 781. In another example, the nucleic acid construct comprises a sequence at least 99%, at least 99.5%, or 100% identical to SEQ ID NO: 764, 765, 780, or 781 and encodes a multidomain therapeutic protein (or a multidomain therapeutic protein comprising a sequence) at least 99%, at least 99.5%, or 100% identical to SEQ ID NO: 746. In another example, the nucleic acid construct comprises a sequence at least 99%, at least 99.5%, or 100% identical to SEQ ID NO: 764, 765, 780, or 781 and encodes a multidomain therapeutic protein comprising the sequence set forth in SEQ ID NO: 746. In another example, the nucleic acid construct comprises the sequence set forth in SEQ ID NO: 764, 765, 780, or 781. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 746 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 746 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 746 (and, e.g., retaining the activity of native GAA). Optionally, the nucleic acid construct in the above examples encodes a multidomain therapeutic protein comprising the sequence set forth in SEQ ID NO: 746. Optionally, the nucleic acid construct in the above examples encodes a multidomain therapeutic protein consisting essentially of the sequence set forth in SEQ ID NO: 746. Optionally, the nucleic acid construct in the above examples encodes a multidomain therapeutic protein consisting of the sequence set forth in SEQ ID NO: 746.

The nucleic acid construct can comprise, for example, (1) a 5′ ITR (e.g., such as the one set forth in SEQ ID NO: 160), (2) a splice acceptor site (e.g., a mouse Alb exon 2 splice acceptor, such as the one set forth in SEQ ID NO: 163), (3) the multidomain therapeutic protein coding sequence, (4) a polyadenylation signal (e.g., a BGH polyadenylation signal, such as the one set forth in SEQ ID NO: 751, or a combination of a BGH polyadenylation signal and a unidirectional SV40 late polyadenylation signal, such as the ones set forth in SEQ ID NOS: 751 and 752, respectively), and (5) a 3′ ITR (e.g., such as the one set forth in SEQ ID NO: 160 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 764 or 780. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 764 or 780 and encodes a multidomain therapeutic protein (or a multidomain therapeutic protein comprising a sequence) at least 99%, at least 99.5%, or 100% identical to SEQ ID NO: 746. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 764 or 780 and encodes a multidomain therapeutic protein comprising the sequence set forth in SEQ ID NO: 746. In another example, the nucleic acid construct comprises a sequence at least 95%, at least 96%, at least 97%, at least 98%, at least 99%, at least 99.5%, or 100% identical to SEQ ID NO: 764 or 780. In another example, the nucleic acid construct comprises a sequence at least 95%, at least 96%, at least 97%, at least 98%, at least 99%, at least 99.5%, or 100% identical to SEQ ID NO: 764 or 780 and encodes a multidomain therapeutic protein (or a multidomain therapeutic protein comprising a sequence) at least 99%, at least 99.5%, or 100% identical to SEQ ID NO: 746. In another example, the nucleic acid construct comprises a sequence at least 95%, at least 96%, at least 97%, at least 98%, at least 99%, at least 99.5%, or 100% identical to SEQ ID NO: 764 or 780 and encodes a multidomain therapeutic protein comprising the sequence set forth in SEQ ID NO: 746. In another example, the nucleic acid construct comprises a sequence at least 99%, at least 99.5%, or 100% identical to SEQ ID NO: 764 or 780. In another example, the nucleic acid construct comprises a sequence at least 99%, at least 99.5%, or 100% identical to SEQ ID NO: 764 or 780 and encodes a multidomain therapeutic protein (or a multidomain therapeutic protein comprising a sequence) at least 99%, at least 99.5%, or 100% identical to SEQ ID NO: 746. In another example, the nucleic acid construct comprises a sequence at least 99%, at least 99.5%, or 100% identical to SEQ ID NO: 764 or 780 and encodes a multidomain therapeutic protein comprising the sequence set forth in SEQ ID NO: 746. In another example, the nucleic acid construct comprises the sequence set forth in SEQ ID NO: 764 or 780. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 746 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 746 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 746 (and, e.g., retaining the activity of native GAA). Optionally, the nucleic acid construct in the above examples encodes a multidomain therapeutic protein comprising the sequence set forth in SEQ ID NO: 746. Optionally, the nucleic acid construct in the above examples encodes a multidomain therapeutic protein consisting essentially of the sequence set forth in SEQ ID NO: 746. Optionally, the nucleic acid construct in the above examples encodes a multidomain therapeutic protein consisting of the sequence set forth in SEQ ID NO: 746.

In a specific example of a multidomain therapeutic protein nucleic acid construct, the encoded multidomain therapeutic protein can comprise SEQ ID NO: 733 or can be at least 90%, at least 91%, at least 92%, at least 93%, at least 94%, at least 95%, at least 96%, at least 97%, at least 98%, at least 99%, or at least 99.5% identical to SEQ ID NO: 733. In another specific example, the multidomain therapeutic protein can consist essentially of SEQ ID NO: 733. In another specific example, the multidomain therapeutic protein can consist of SEQ ID NO: 733.

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 756, 757, 758, and 736. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 756, 757, 758, and 736. In another example, the multidomain therapeutic protein coding sequence is (or comprises a sequence) at least 99%, at least 99.5%, or 100% identical to any one of SEQ ID NOS: 756, 757, 758, and 736. In another example, the multidomain therapeutic protein coding sequence comprises the sequence set forth in any one of SEQ ID NOS: 756, 757, 758, and 736. In another example, the multidomain therapeutic protein coding sequence consists essentially of the sequence set forth in any one of SEQ ID NOS: 756, 757, 758, and 736. In another example, the multidomain therapeutic protein coding sequence consists of the sequence set forth in any one of SEQ ID NOS: 756, 757, 758, and 736. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 733 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 733 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 733 (and, e.g., retaining the activity of native GAA). Optionally, the multidomain therapeutic protein coding sequence in the above examples encodes a multidomain therapeutic protein comprising the sequence set forth in SEQ ID NO: 733. Optionally, the multidomain therapeutic protein coding sequence in the above examples encodes a multidomain therapeutic protein consisting essentially of the sequence set forth in SEQ ID NO: 733. Optionally, the multidomain therapeutic protein coding sequence in the above examples encodes a multidomain therapeutic protein consisting of the sequence set forth in SEQ ID NO: 733. In some embodiments, the nucleotide at position 3 is an “A.” In some embodiments, the nucleotide at position 132 is an “A.” In some embodiments, the nucleotide at position 273 is a “T.” In some embodiments, the nucleotide at position 723 is a “G.” In some embodiments, the nucleotide at position 1830 is a “G.” In some embodiments, the nucleotide at position 1833 is a “C.” In some embodiments, the nucleotide at position 3078 is a “G.” In some embodiments, the nucleotide at position 3 is an “A,” the nucleotide at position 132 is an “A,” the nucleotide at position 273 is a “T,” the nucleotide at position 723 is a “G,” the nucleotide at position 1830 is a “G,” the nucleotide at position 1833 is a “C,” and the nucleotide at position 3078 is a “G.” In some embodiments, the nucleotide at position 273 is a “T,” the nucleotide at position 723 is a “G,” the nucleotide at position 1830 is a “G,” the nucleotide at position 1833 is a “C,” and the nucleotide at position 3078 is a “G.” In some embodiments, the nucleotide at position 1830 is a “G,” the nucleotide at position 1833 is a “C,” and the nucleotide at position 3078 is a “G.”

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 756. In another example, the multidomain therapeutic protein coding sequence is (or comprises a sequence) at least 95%, at least 96%, at least 97%, at least 98%, at least 99%, at least 99.5%, or 100% identical to SEQ ID NO: 756. In another example, the multidomain therapeutic protein coding sequence is (or comprises a sequence) at least 99%, at least 99.5%, or 100% identical to SEQ ID NO: 756. In another example, the multidomain therapeutic protein coding sequence comprises the sequence set forth in SEQ ID NO: 756. In another example, the multidomain therapeutic protein coding sequence consists essentially of the sequence set forth in SEQ ID NO: 756. In another example, the multidomain therapeutic protein coding sequence consists of the sequence set forth in SEQ ID NO: 756. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 733 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 733 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 733 (and, e.g., retaining the activity of native GAA). Optionally, the multidomain therapeutic protein coding sequence in the above examples encodes a multidomain therapeutic protein comprising the sequence set forth in SEQ ID NO: 733. Optionally, the multidomain therapeutic protein coding sequence in the above examples encodes a multidomain therapeutic protein consisting essentially of the sequence set forth in SEQ ID NO: 733. Optionally, the multidomain therapeutic protein coding sequence in the above examples encodes a multidomain therapeutic protein consisting of the sequence set forth in SEQ ID NO: 733. In some embodiments, the nucleotide at position 3 is an “A.” In some embodiments, the nucleotide at position 132 is an “A.” In some embodiments, the nucleotide at position 273 is a “T.” In some embodiments, the nucleotide at position 723 is a “G.” In some embodiments, the nucleotide at position 1830 is a “G.” In some embodiments, the nucleotide at position 1833 is a “C.” In some embodiments, the nucleotide at position 3078 is a “G.” In some embodiments, the nucleotide at position 3 is an “A,” the nucleotide at position 132 is an “A,” the nucleotide at position 273 is a “T,” the nucleotide at position 723 is a “G,” the nucleotide at position 1830 is a “G,” the nucleotide at position 1833 is a “C,” and the nucleotide at position 3078 is a “G.” In some embodiments, the nucleotide at position 273 is a “T,” the nucleotide at position 723 is a “G,” the nucleotide at position 1830 is a “G,” the nucleotide at position 1833 is a “C,” and the nucleotide at position 3078 is a “G.” In some embodiments, the nucleotide at position 1830 is a “G,” the nucleotide at position 1833 is a “C,” and the nucleotide at position 3078 is a “G.”

The nucleic acid construct can comprise, for example, (1) a 5′ ITR (e.g., such as the one set forth in SEQ ID NO: 160), (2) a splice acceptor site (e.g., a mouse Alb exon 2 splice acceptor, such as the one set forth in SEQ ID NO: 163), (3) the multidomain therapeutic protein coding sequence, (4) a polyadenylation signal (e.g., a BGH polyadenylation signal, such as the one set forth in SEQ ID NO: 751, or a combination of a BGH polyadenylation signal and a unidirectional SV40 late polyadenylation signal, such as the ones set forth in SEQ ID NOS: 751 and 752, respectively), and (5) a 3′ ITR (e.g., such as the one set forth in SEQ ID NO: 160 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 777, 778, 793, or 794. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 777, 778, 793, or 794 and encodes a multidomain therapeutic protein (or a multidomain therapeutic protein comprising a sequence) at least 99%, at least 99.5%, or 100% identical to SEQ ID NO: 733. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 777, 778, 793, or 794 and encodes a multidomain therapeutic protein comprising the sequence set forth in SEQ ID NO: 733. In another example, the nucleic acid construct comprises a sequence at least 95%, at least 96%, at least 97%, at least 98%, at least 99%, at least 99.5%, or 100% identical to SEQ ID NO: 777, 778, 793, or 794. In another example, the nucleic acid construct comprises a sequence at least 95%, at least 96%, at least 97%, at least 98%, at least 99%, at least 99.5%, or 100% identical to SEQ ID NO: 777, 778, 793, or 794 and encodes a multidomain therapeutic protein (or a multidomain therapeutic protein comprising a sequence) at least 99%, at least 99.5%, or 100% identical to SEQ ID NO: 733. In another example, the nucleic acid construct comprises a sequence at least 95%, at least 96%, at least 97%, at least 98%, at least 99%, at least 99.5%, or 100% identical to SEQ ID NO: 777, 778, 793, or 794 and encodes a multidomain therapeutic protein comprising the sequence set forth in SEQ ID NO: 733. In another example, the nucleic acid construct comprises a sequence at least 99%, at least 99.5%, or 100% identical to SEQ ID NO: 777, 778, 793, or 794. In another example, the nucleic acid construct comprises a sequence at least 99%, at least 99.5%, or 100% identical to SEQ ID NO: 777, 778, 793, or 794 and encodes a multidomain therapeutic protein (or a multidomain therapeutic protein comprising a sequence) at least 99%, at least 99.5%, or 100% identical to SEQ ID NO: 733. In another example, the nucleic acid construct comprises a sequence at least 99%, at least 99.5%, or 100% identical to SEQ ID NO: 777, 778, 793, or 794 and encodes a multidomain therapeutic protein comprising the sequence set forth in SEQ ID NO: 733. In another example, the nucleic acid construct comprises the sequence set forth in SEQ ID NO: 777, 778, 793, or 794. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 733 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 733 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 733 (and, e.g., retaining the activity of native GAA). Optionally, the nucleic acid construct in the above examples encodes a multidomain therapeutic protein comprising the sequence set forth in SEQ ID NO: 733. Optionally, the nucleic acid construct in the above examples encodes a multidomain therapeutic protein consisting essentially of the sequence set forth in SEQ ID NO: 733. Optionally, the nucleic acid construct in the above examples encodes a multidomain therapeutic protein consisting of the sequence set forth in SEQ ID NO: 733.

The nucleic acid construct can comprise, for example, (1) a 5′ ITR (e.g., such as the one set forth in SEQ ID NO: 160), (2) a splice acceptor site (e.g., a mouse Alb exon 2 splice acceptor, such as the one set forth in SEQ ID NO: 163), (3) the multidomain therapeutic protein coding sequence, (4) a polyadenylation signal (e.g., a BGH polyadenylation signal, such as the one set forth in SEQ ID NO: 751, or a combination of a BGH polyadenylation signal and a unidirectional SV40 late polyadenylation signal, such as the ones set forth in SEQ ID NOS: 751 and 752, respectively), and (5) a 3′ ITR (e.g., such as the one set forth in SEQ ID NO: 160 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 777 or 793. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 777 or 793 and encodes a multidomain therapeutic protein (or a multidomain therapeutic protein comprising a sequence) at least 99%, at least 99.5%, or 100% identical to SEQ ID NO: 733. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 777 or 793 and encodes a multidomain therapeutic protein comprising the sequence set forth in SEQ ID NO: 733. In another example, the nucleic acid construct comprises a sequence at least 95%, at least 96%, at least 97%, at least 98%, at least 99%, at least 99.5%, or 100% identical to SEQ ID NO: 777 or 793. In another example, the nucleic acid construct comprises a sequence at least 95%, at least 96%, at least 97%, at least 98%, at least 99%, at least 99.5%, or 100% identical to SEQ ID NO: 777 or 793 and encodes a multidomain therapeutic protein (or a multidomain therapeutic protein comprising a sequence) at least 99%, at least 99.5%, or 100% identical to SEQ ID NO: 733. In another example, the nucleic acid construct comprises a sequence at least 95%, at least 96%, at least 97%, at least 98%, at least 99%, at least 99.5%, or 100% identical to SEQ ID NO: 777 or 793 and encodes a multidomain therapeutic protein comprising the sequence set forth in SEQ ID NO: 733. In another example, the nucleic acid construct comprises a sequence at least 99%, at least 99.5%, or 100% identical to SEQ ID NO: 777 or 793. In another example, the nucleic acid construct comprises a sequence at least 99%, at least 99.5%, or 100% identical to SEQ ID NO: 777 or 793 and encodes a multidomain therapeutic protein (or a multidomain therapeutic protein comprising a sequence) at least 99%, at least 99.5%, or 100% identical to SEQ ID NO: 733. In another example, the nucleic acid construct comprises a sequence at least 99%, at least 99.5%, or 100% identical to SEQ ID NO: 777 or 793 and encodes a multidomain therapeutic protein comprising the sequence set forth in SEQ ID NO: 733. In another example, the nucleic acid construct comprises the sequence set forth in SEQ ID NO: 777 or 793. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 733 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 733 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 733 (and, e.g., retaining the activity of native GAA). Optionally, the nucleic acid construct in the above examples encodes a multidomain therapeutic protein comprising the sequence set forth in SEQ ID NO: 733. Optionally, the nucleic acid construct in the above examples encodes a multidomain therapeutic protein consisting essentially of the sequence set forth in SEQ ID NO: 733. Optionally, the nucleic acid construct in the above examples encodes a multidomain therapeutic protein consisting of the sequence set forth in SEQ ID NO: 733.

When specific multidomain therapeutic protein nucleic acid constructs sequences are disclosed herein, they are meant to encompass the sequence disclosed or the reverse complement of the sequence. For example, if a multidomain therapeutic protein nucleic acid construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the multidomain therapeutic protein nucleic acid constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### (5) Vectors

The nucleic acid constructs disclosed herein can be provided in a vector for expression or for integration into and expression from a target genomic locus. A vector can comprise additional sequences such as, for example, replication origins, promoters, and genes encoding antibiotic resistance. A vector can also comprise nuclease agent components as disclosed elsewhere herein. For example, a vector can comprise a nucleic acid construct encoding a multidomain therapeutic protein, a CRISPR/Cas system (nucleic acids encoding Cas protein and gRNA), one or more components of a CRISPR/Cas system, or a combination thereof (e.g., a nucleic acid construct and a gRNA). In some cases, a vector comprising a nucleic acid construct encoding a multidomain therapeutic protein does not comprise any components of the nuclease agents described herein (e.g., does not comprise a nucleic acid encoding a Cas protein and does not comprise a nucleic acid encoding a gRNA). Some such vectors comprise homology arms corresponding to target sites in the target genomic locus. Other such vectors do not comprise any homology arms.

Some vectors may be circular. Alternatively, the vector may be linear. The vector can be packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression or longer-lasting expression. Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹² to about 10¹⁶ vg/mL. Other exemplary viral titers (e.g., AAV titers) include about 10¹² to about 10¹⁶ vg/kg of body weight.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. Indeed, rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In therapeutic rAAV genomes, a gene expression cassette is placed between ITR sequences. Typically, rAAV genome cassettes comprise of a promoter to drive expression of a therapeutic transgene, followed by polyadenylation sequence. The ITRs flanking a rAAV expression cassette are usually derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

Some non-limiting examples of ITRs that can be used include ITRs comprising, consisting essentially of, or consisting of SEQ ID NO: 158, SEQ ID NO: 159, or SEQ ID NO: 160. Other examples of ITRs comprise one or more mutations compared to SEQ ID NO: 158, SEQ ID NO: 159, or SEQ ID NO: 160 and can be at least 90%, at least 91%, at least 92%, at least 93%, at least 94%, at least 95%, at least 96%, at least 97%, at least 98%, or at least 99% identical to SEQ ID NO: 158, SEQ ID NO: 159, or SEQ ID NO: 160. In some rAAV genomes disclosed herein, the nucleic acid construct is flanked on both sides by the same ITR (i.e., the ITR on the 5′ end, and the reverse complement of the ITR on the 3′ end, such as SEQ ID NO: 158 on the 5′ end and SEQ ID NO: 168 on the 3′ end, or SEQ ID NO: 159 on the 5′ end and SEQ ID NO: 613 on the 3′ end, or SEQ ID NO: 160 on the 5′ end and SEQ ID NO: 614 on the 3′ end). In one example, the ITR on each end can comprise, consist essentially of, or consist of SEQ ID NO: 158 (i.e., SEQ ID NO: 158 on the 5′ end, and the reverse complement on the 3′ end). In another example, the ITR on each end can comprise, consist essentially of, or consist of SEQ ID NO: 159 (i.e., SEQ ID NO: 159 on the 5′ end, and the reverse complement on the 3′ end). In one example, the ITR on at least one end comprises, consists essentially of, or consists of SEQ ID NO: 160. In one example, the ITR on the 5′ end comprises, consists essentially of, or consists of SEQ ID NO: 160. In one example, the ITR on the 3′ end comprises, consists essentially of, or consists of SEQ ID NO: 160. In one example, the ITR on each end can comprise, consist essentially of, or consist of SEQ ID NO: 160 (i.e., SEQ ID NO: 160 on the 5′ end, and the reverse complement on the 3′ end). In other rAAV genomes disclosed herein, the nucleic acid construct is flanked by different ITRs on each end. In one example, the ITR on one end comprises, consists essentially of, or consists of SEQ ID NO: 158, and the ITR on the other end comprises, consists essentially of, or consists of SEQ ID NO: 159. In another example, the ITR on one end comprises, consists essentially of, or consists of SEQ ID NO: 158, and the ITR on the other end comprises, consists essentially of, or consists of SEQ ID NO: 160. In one example, the ITR on one end comprises, consists essentially of, or consists of SEQ ID NO: 159, and the ITR on the other end comprises, consists essentially of, or consists of SEQ ID NO: 160.

The specific serotype of a recombinant AAV vector influences its in vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo. Several serotypes of rAAVs, including rAAV8, are capable of transducing the liver when delivered systemically in mice, NHPs and humans. See, e.g., Li et al. (2020) Nat. Rev. Genet. 21:255-272, herein incorporated by reference in its entirety for all purposes.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

When specific rAAVs comprising specific sequences (e.g., specific bidirectional construct sequences or specific unidirectional construct sequences) are disclosed herein, they are meant to encompass the sequence disclosed or the reverse complement of the sequence. For example, if a bidirectional or unidirectional construct disclosed herein consists of the hypothetical sequence 5′-CTGGACCGA-3′, it is also meant to encompass the reverse complement of that sequence (5′-TCGGTCCAG-3′). Likewise, when rAAVs comprising bidirectional or unidirectional construct elements in a specific 5′ to 3′ order are disclosed herein, they are also meant to encompass the reverse complement of the order of those elements. For example, if an rAAV is disclosed herein that comprises a bidirectional construct that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. An “AAV vector” as used herein refers to an AAV vector comprising a heterologous sequence not of AAV origin (i.e., a nucleic acid sequence heterologous to AAV), typically comprising a sequence encoding an exogenous polypeptide of interest (e.g., multidomain therapeutic protein). The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for liver tissue include AAV3B, AAV5, AAV6, AAV7, AAV8, AAV9, AAVrh.74, and AAVhu.37, and particularly AAV8. In a specific example, the AAV vector comprising the nucleic acid construct can be recombinant AAV8 (rAAV8). A rAAV8 vector as described herein is one in which the capsid is from AAV8. For example, an AAV vector using ITRs from AAV2 and a capsid of AAV8 is considered herein to be a rAAV8 vector.

Tropism can be further refined through pseudotyping, which is the mixing of a capsid and a genome from different viral serotypes. For example, AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

### B. Nuclease Agents and CRISPR/Cas Systems

The methods and compositions disclosed herein can utilize nuclease agents such as Clustered Regularly Interspersed Short Palindromic Repeats (CRISPR)/CRISPR-associated (Cas) systems, zinc finger nuclease (ZFN) systems, or Transcription Activator-Like Effector Nuclease (TALEN) systems or components of such systems to modify a target genomic locus in a target gene such as a safe harbor gene (e.g., ALB) for insertion of a nucleic acid construct as disclosed herein. Generally, the nuclease agents involve the use of engineered cleavage systems to induce a double strand break or a nick (i.e., a single strand break) in a nuclease target site. Cleavage or nicking can occur through the use of specific nucleases such as engineered ZFNs, TALENs, or CRISPR/Cas systems with an engineered guide RNA to guide specific cleavage or nicking of the nuclease target site. Any nuclease agent that induces a nick or double-strand break at a desired target sequence can be used in the methods and compositions disclosed herein. The nuclease agent can be used to create a site of insertion at a desired locus (target gene) within a host genome, at which site the nucleic acid construct is inserted to express the multidomain therapeutic protein.

In one example, the nuclease agent is a CRISPR/Cas system. In another example, the nuclease agent comprises one or more ZFNs. In yet another example, the nuclease agent comprises one or more TALENs. In a specific example, the CRISPR/Cas systems or components of such systems target an ALB gene or locus (e.g., ALB genomic locus) within a cell, or intron 1 of an ALB gene or locus within a cell. In a more specific example, the CRISPR/Cas systems or components of such systems target a human ALB gene or locus or intron 1 of a human ALB gene or locus within a cell.

CRISPR/Cas systems include transcripts and other elements involved in the expression of, or directing the activity of, Cas genes. A CRISPR/Cas system can be, for example, a type I, a type II, a type III system, or a type V system (e.g., subtype V-A or subtype V-B). The methods and compositions disclosed herein can employ CRISPR/Cas systems by utilizing CRISPR complexes (comprising a guide RNA (gRNA) complexed with a Cas protein) for site-directed binding or cleavage of nucleic acids. A CRISPR/Cas system targeting an ALB gene or locus comprises a Cas protein (or a nucleic acid encoding the Cas protein) and one or more guide RNAs (or DNAs encoding the one or more guide RNAs), with each of the one or more guide RNAs targeting a different guide RNA target sequence in the target genomic locus (e.g., ALB gene or locus).

CRISPR/Cas systems used in the compositions and methods disclosed herein can be non-naturally occurring. A non-naturally occurring system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together, employ a Cas protein that does not occur naturally, or employ a gRNA that does not occur naturally.

### (1) Target Genomic Loci and Albumin (ALB)

Any target genomic locus capable of expressing a gene can be used, such as a safe harbor locus (safe harbor gene, such as ALB) or an endogenous GAA locus. The nucleic acid construct can be integrated into any part of the target genomic locus. For example, the nucleic acid construct can be inserted into an intron or an exon of a target genomic locus or can replace one or more introns and/or exons of a target genomic locus. In a specific example, the nucleic acid construct can be integrated into an intron of the target genomic locus, such as the first intron of the target genomic locus (e.g., ALB intron 1). See, e.g., WO 2020/082042, US 2020/0270617, WO 2020/082041, US 2020/0268906, WO 2020/082046, and US 2020/0289628, each of which is herein incorporated by reference in its entirety for all purposes. Constructs integrated into a target genomic locus can be operably linked to an endogenous promoter at the target genomic locus (e.g., the endogenous ALB promoter).

Interactions between integrated exogenous DNA and a host genome can limit the reliability and safety of integration and can lead to overt phenotypic effects that are not due to the targeted genetic modification but are instead due to unintended effects of the integration on surrounding endogenous genes. For example, randomly inserted transgenes can be subject to position effects and silencing, making their expression unreliable and unpredictable. Likewise, integration of exogenous DNA into a chromosomal locus can affect surrounding endogenous genes and chromatin, thereby altering cell behavior and phenotypes. Safe harbor loci include chromosomal loci where transgenes or other exogenous nucleic acid inserts can be stably and reliably expressed in all tissues of interest without overtly altering cell behavior or phenotype (i.e., without any deleterious effects on the host cell). See, e.g., Sadelain et al. (2012) Nat. Rev. Cancer 12:51-58, herein incorporated by reference in its entirety for all purposes. For example, the safe harbor locus can be one in which expression of the inserted gene sequence is not perturbed by any read-through expression from neighboring genes. For example, safe harbor loci can include chromosomal loci where exogenous DNA can integrate and function in a predictable manner without adversely affecting endogenous gene structure or expression. Safe harbor loci can include extragenic regions or intragenic regions such as, for example, loci within genes that are non-essential, dispensable, or able to be disrupted without overt phenotypic consequences.

Such safe harbor loci can offer an open chromatin configuration in all tissues and can be ubiquitously expressed during embryonic development and in adults. See, e.g., Zambrowicz et al. (1997) Proc. Natl. Acad. Sci. U.S.A. 94:3789-3794, herein incorporated by reference in its entirety for all purposes. In addition, the safe harbor loci can be targeted with high efficiency, and safe harbor loci can be disrupted with no overt phenotype. Examples of safe harbor loci include ALB, CCR5, HPRT, AAVS1, and Rosa26. See, e.g., U.S. Pat. Nos. 7,888,121; 7,972,854; 7,914,796; 7,951,925; 8,110,379; 8,409,861; 8,586,526; and US Patent Publication Nos. 2003/0232410; 2005/0208489; 2005/0026157; 2006/0063231; 2008/0159996; 2010/00218264; 2012/0017290; 2011/0265198; 2013/0137104; 2013/0122591; 2013/0177983; 2013/0177960; and 2013/0122591, each of which is herein incorporated by reference in its entirety for all purposes. Other examples of target genomic loci include an ALB locus, a EESYR locus, a SARS locus, position 188,083,272 of human chromosome 1 or its non-human mammalian orthologue, position 3,046,320 of human chromosome 10 or its non-human mammalian orthologue, position 67, 328,980 of human chromosome 17 or its non-human mammalian orthologue, an adeno-associated virus site 1 (AAVS1) on chromosome, a naturally occurring site of integration of AAV virus on human chromosome 19 or its non-human mammalian orthologue, a chemokine receptor 5 (CCR5) gene, a chemokine receptor gene encoding an HIV-1 coreceptor, or a mouse Rosa26 locus or its non-murine mammalian orthologue.

In a specific example, a safe harbor locus is a locus within the genome wherein a gene may be inserted without significant deleterious effects on the host cell such as a hepatocyte (e.g., without causing apoptosis, necrosis, and/or senescence, or without causing more than 5%, 10%, 15%, 20%, 25%, 30%, or 40% apoptosis, necrosis, and/or senescence as compared to a control population of cells). The safe harbor locus can allow overexpression of an exogenous gene without significant deleterious effects on the host cell such as a hepatocyte (e.g., without causing apoptosis, necrosis, and/or senescence, or without causing more than 5%, 10%, 15%, 20%, 25%, 30%, or 40% apoptosis, necrosis, and/or senescence as compared to a control population of cells). A desirable safe harbor locus may be one in which expression of the inserted gene sequence is not perturbed by read-through expression from neighboring genes. The safe harbor may be a human safe harbor (e.g., for a liver tissue or hepatocyte host cell).

In a specific example, the target genomic locus is an ALB locus, such as intron 1 of an ALB locus. In a more specific example, the target genomic locus is a human ALB locus, such as intron 1 of a human ALB locus (e.g., SEQ ID NO: 4).

### (2) Cas Proteins

Cas proteins generally comprise at least one RNA recognition or binding domain that can interact with guide RNAs. Cas proteins can also comprise nuclease domains (e.g., DNase domains or Rnase domains), DNA-binding domains, helicase domains, protein-protein interaction domains, dimerization domains, and other domains. Some such domains (e.g., Dnase domains) can be from a native Cas protein. Other such domains can be added to make a modified Cas protein. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18^{th} base pair from the PAM sequence on the non-targeted strand and after the 23^{rd} base on the targeted strand. A Cas protein can have full cleavage activity to create a double-strand break at a target genomic locus (e.g., a double-strand break with blunt ends), or it can be a nickase that creates a single-strand break at a target genomic locus.

Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

An exemplary Cas protein is a Cas9 protein or a protein derived from a Cas9 protein. Cas9 proteins are from a type II CRISPR/Cas system and typically share four key motifs with a conserved architecture. Motifs 1, 2, and 4 are RuvC-like motifs, and motif 3 is an HNH motif. Exemplary Cas9 proteins are from Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Cantidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Neisseria meningitidis, or Campylobacter jejuni. Additional examples of the Cas9 family members are described in WO 2014/131833, herein incorporated by reference in its entirety for all purposes. Cas9 from S. pyogenes (SpCas9) (e.g., assigned UniProt accession number Q99ZW2) is an exemplary Cas9 protein. An exemplary SpCas9 protein sequence is set forth in SEQ ID NO: 8 (encoded by the DNA sequence set forth in SEQ ID NO: 9). An exemplary SpCas9 mRNA (cDNA) sequence is set forth in SEQ ID NO: 10. Smaller Cas9 proteins (e.g., Cas9 proteins whose coding sequences are compatible with the maximum AAV packaging capacity when combined with a guide RNA coding sequence and regulatory elements for the Cas9 and guide RNA, such as SaCas9 and CjCas9 and Nme2Cas9) are other exemplary Cas9 proteins. For example, Cas9 from S. aureus (SaCas9) (e.g., assigned UniProt accession number J7RUA5) is another exemplary Cas9 protein. Likewise, Cas9 from Campylobacter jejuni (CjCas9) (e.g., assigned UniProt accession number Q0P897) is another exemplary Cas9 protein. See, e.g., Kim et al. (2017) Nat. Commun. 8:14500, herein incorporated by reference in its entirety for all purposes. SaCas9 is smaller than SpCas9, and CjCas9 is smaller than both SaCas9 and SpCas9. Cas9 from Neisseria meningitidis (Nme2Cas9) is another exemplary Cas9 protein. See, e.g., Edraki et al. (2019) Mol. Cell 73(4):714-726, herein incorporated by reference in its entirety for all purposes. Cas9 proteins from Streptococcus thermophilus (e.g., Streptococcus thermophilus LMD-9 Cas9 encoded by the CRISPR1 locus (St1Cas9) or Streptococcus thermophilus Cas9 from the CRISPR3 locus (St3Cas9)) are other exemplary Cas9 proteins. Cas9 from Francisella novicida (FnCas9) or the RHA Francisella novicida Cas9 variant that recognizes an alternative PAM (E1369R/E1449H/R1556A substitutions) are other exemplary Cas9 proteins. These and other exemplary Cas9 proteins are reviewed, e.g., in Cebrian-Serrano and Davies (2017) Mamm. Genome 28(7):247-261, herein incorporated by reference in its entirety for all purposes. Examples of Cas9 coding sequences, Cas9 mRNAs, and Cas9 protein sequences are provided in WO 2013/176772, WO 2014/065596, WO 2016/106121, WO 2019/067910, WO 2020/082042, US 2020/0270617, WO 2020/082041, US 2020/0268906, WO 2020/082046, and US 2020/0289628, each of which is herein incorporated by reference in its entirety for all purposes. Specific examples of ORFs and Cas9 amino acid sequences are provided in Table 30 at paragraph [0449] WO 2019/067910, and specific examples of Cas9 mRNAs and ORFs are provided in paragraphs [0214]-[0234] of WO 2019/067910. See also WO 2020/082046 A2 (pp. 84-85) and Table 24 in WO 2020/069296, each of which is herein incorporated by reference in its entirety for all purposes. An exemplary SpCas9 protein sequence comprises, consists essentially of, or consists of SEQ ID NO: 11. An exemplary SpCas9 mRNA sequence encoding that SpCas9 protein sequence comprises, consists essentially of, or consists of SEQ ID NO: 12. Another exemplary SpCas9 mRNA sequence encoding that SpCas9 protein sequence comprises, consists essentially of, or consists of SEQ ID NO: 1. Another exemplary SpCas9 mRNA sequence encoding that SpCas9 protein sequence comprises SEQ ID NO: 2. An exemplary SpCas9 coding sequence comprises, consists essentially of, or consists of SEQ ID NO: 3.

Another example of a Cas protein is a Cpf1 (CRISPR from Prevotella and Francisella 1) protein. Cpf1 is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in Cas9 proteins, and the RuvC-like domain is contiguous in the Cpf1 sequence, in contrast to Cas9 where it contains long inserts including the HNH domain. See, e.g., Zetsche et al. (2015) Cell 163(3):759-771, herein incorporated by reference in its entirety for all purposes. Exemplary Cpf1 proteins are from Francisella tularensis 1, Francisella tularensis subsp. Novicida, Prevotella albensis, Lachnospiraceae bacterium MC20171, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Cantidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. Cpf1 from Francisella novicida U112 (FnCpf1; assigned UniProt accession number AOQ7Q2) is an exemplary Cpf1 protein.

Another example of a Cas protein is CasX (Cas12e). CasX is an RNA-guided DNA endonuclease that generates a staggered double-strand break in DNA. CasX is less than 1000 amino acids in size. Exemplary CasX proteins are from Deltaproteobacteria (DpbCasX or DpbCas12e) and Planctomycetes (PlmCasX or PlmCas12e). Like Cpf1, CasX uses a single RuvC active site for DNA cleavage. See, e.g., Liu et al. (2019) Nature 566(7743):218-223, herein incorporated by reference in its entirety for all purposes.

Another example of a Cas protein is CasΦ (CasPhi or Cas12j), which is uniquely found in bacteriophages. CasΦ is less than 1000 amino acids in size (e.g., 700-800 amino acids). CasΦ cleavage generates staggered 5′ overhangs. A single RuvC active site in CasΦ is capable of crRNA processing and DNA cutting. See, e.g., Pausch et al. (2020) Science 369(6501):333-337, herein incorporated by reference in its entirety for all purposes.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

One example of a modified Cas protein is the modified SpCas9-HF1 protein, which is a high-fidelity variant of Streptococcus pyogenes Cas9 harboring alterations (N497A/R661A/Q695A/Q926A) designed to reduce non-specific DNA contacts. See, e.g., Kleinstiver et al. (2016) Nature 529(7587):490-495, herein incorporated by reference in its entirety for all purposes. Another example of a modified Cas protein is the modified eSpCas9 variant (K848A/K1003A/R1060A) designed to reduce off-target effects. See, e.g., Slaymaker et al. (2016) Science 351(6268):84-88, herein incorporated by reference in its entirety for all purposes. Other SpCas9 variants include K855A and K810A/K1003A/R1060A. These and other modified Cas proteins are reviewed, e.g., in Cebrian-Serrano and Davies (2017) Mamm. Genome 28(7):247-261, herein incorporated by reference in its entirety for all purposes. Another example of a modified Cas9 protein is xCas9, which is a SpCas9 variant that can recognize an expanded range of PAM sequences. See, e.g., Hu et al. (2018) Nature 556:57-63, herein incorporated by reference in its entirety for all purposes.

Cas proteins can be modified to increase or decrease one or more of nucleic acid binding affinity, nucleic acid binding specificity, and enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of or a property of the Cas protein.

Cas proteins can comprise at least one nuclease domain, such as a Dnase domain. For example, a wild type Cpf1 protein generally comprises a RuvC-like domain that cleaves both strands of target DNA, perhaps in a dimeric configuration. Likewise, CasX and CasΦ D generally comprise a single RuvC-like domain that cleaves both strands of a target DNA. Cas proteins can also comprise at least two nuclease domains, such as Dnase domains. For example, a wild type Cas9 protein generally comprises a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337(6096):816-821, herein incorporated by reference in its entirety for all purposes.

One or more of the nuclease domains can be deleted or mutated so that they are no longer functional or have reduced nuclease activity. For example, if one of the nuclease domains is deleted or mutated in a Cas9 protein, the resulting Cas9 protein can be referred to as a nickase and can generate a single-strand break within a double-stranded target DNA but not a double-strand break (i.e., it can cleave the complementary strand or the non-complementary strand, but not both). If none of the nuclease domains is deleted or mutated in a Cas9 protein, the Cas9 protein will retain double-strand-break-inducing activity. An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839), H840A (histidine to alanine at amino acid position 840), or N863A (asparagine to alanine at amino acid position N863) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Res. 39(21):9275-9282 and WO 2013/141680, each of which is herein incorporated by reference in its entirety for all purposes. Such mutations can be generated using methods such as site-directed mutagenesis, PCR-mediated mutagenesis, or total gene synthesis. Examples of other mutations creating nickases can be found, for example, in WO 2013/176772 and WO 2013/142578, each of which is herein incorporated by reference in its entirety for all purposes.

Examples of inactivating mutations in the catalytic domains of xCas9 are the same as those described above for SpCas9. Examples of inactivating mutations in the catalytic domains of Staphylococcus aureus Cas9 proteins are also known. For example, the Staphylococcus aureus Cas9 enzyme (SaCas9) may comprise a substitution at position N580 (e.g., N580A substitution) or a substitution at position D10 (e.g., D10A substitution) to generate a Cas nickase. See, e.g., WO 2016/106236, herein incorporated by reference in its entirety for all purposes. Examples of inactivating mutations in the catalytic domains of Nme2Cas9 are also known (e.g., D16A or H588A). Examples of inactivating mutations in the catalytic domains of St1Cas9 are also known (e.g., D9A, D598A, H599A, or N622A). Examples of inactivating mutations in the catalytic domains of St3Cas9 are also known (e.g., D10A or N870A). Examples of inactivating mutations in the catalytic domains of CjCas9 are also known (e.g., combination of D8A or H559A). Examples of inactivating mutations in the catalytic domains of FnCas9 and RHA FnCas9 are also known (e.g., N995A).

Examples of inactivating mutations in the catalytic domains of Cpf1 proteins are also known. With reference to Cpf1 proteins from Francisella novicida U112 (FnCpf1), Acidaminococcus sp. BV3L6 (AsCpf1), Lachnospiraceae bacterium ND2006 (LbCpf1), and Moraxella bovoculi 237 (MbCpf1 Cpf1), such mutations can include mutations at positions 908, 993, or 1263 of AsCpf1 or corresponding positions in Cpf1 orthologs, or positions 832, 925, 947, or 1180 of LbCpf1 or corresponding positions in Cpf1 orthologs. Such mutations can include, for example, one or more of mutations D908A, E993A, and D1263A of AsCpf1 or corresponding mutations in Cpf1 orthologs, or D832A, E925A, D947A, and D1180A of LbCpf1 or corresponding mutations in Cpf1 orthologs. See, e.g., US 2016/0208243, herein incorporated by reference in its entirety for all purposes.

Examples of inactivating mutations in the catalytic domains of CasX proteins are also known. With reference to CasX proteins from Deltaproteobacteria, D672A, E769A, and D935A (individually or in combination) or corresponding positions in other CasX orthologs are inactivating. See, e.g., Liu et al. (2019) Nature 566(7743):218-223, herein incorporated by reference in its entirety for all purposes.

Examples of inactivating mutations in the catalytic domains of CasΦ D proteins are also known. For example, D371A and D394A, alone or in combination, are inactivating mutations. See, e.g., Pausch et al. (2020) Science 369(6501):333-337, herein incorporated by reference in its entirety for all purposes.

Cas proteins can also be operably linked to heterologous polypeptides as fusion proteins. For example, a Cas protein can be fused to a cleavage domain. See WO 2014/089290, herein incorporated by reference in its entirety for all purposesCas proteins can also be fused to a heterologous polypeptide providing increased or decreased stability. The fused domain or heterologous polypeptide can be located at the N-terminus, the C-terminus, or internally within the Cas protein.

As one example, a Cas protein can be fused to one or more heterologous polypeptides that provide for subcellular localization. Such heterologous polypeptides can include, for example, one or more nuclear localization signals (NLS) such as the monopartite SV40 NLS and/or a bipartite alpha-importin NLS for targeting to the nucleus, a mitochondrial localization signal for targeting to the mitochondria, an ER retention signal, and the like. See, e.g., Lange et al. (2007) J. Biol. Chem. 282(8):5101-5105, herein incorporated by reference in its entirety for all purposes. Such subcellular localization signals can be located at the N-terminus, the C-terminus, or anywhere within the Cas protein. An NLS can comprise a stretch of basic amino acids, and can be a monopartite sequence or a bipartite sequence. Optionally, a Cas protein can comprise two or more NLSs, including an NLS (e.g., an alpha-importin NLS or a monopartite NLS) at the N-terminus and an NLS (e.g., an SV40 NLS or a bipartite NLS) at the C-terminus. A Cas protein can also comprise two or more NLSs at the N-terminus and/or two or more NLSs at the C-terminus.

A Cas protein may, for example, be fused with 1-10 NLSs (e.g., fused with 1-5 NLSs or fused with one NLS. Where one NLS is used, the NLS may be linked at the N-terminus or the C-terminus of the Cas protein sequence. It may also be inserted within the Cas protein sequence. Alternatively, the Cas protein may be fused with more than one NLS. For example, the Cas protein may be fused with 2, 3, 4, or 5 NLSs. In a specific example, the Cas protein may be fused with two NLSs. In certain circumstances, the two NLSs may be the same (e.g., two SV40 NLSs) or different. For example, the Cas protein can be fused to two SV40 NLS sequences linked at the carboxy terminus. Alternatively, the Cas protein may be fused with two NLSs, one linked at the N-terminus and one at the C-terminus. In other examples, the Cas protein may be fused with 3 NLSs or with no NLS. The NLS may be a monopartite sequence, such as, e.g., the SV40 NLS, PKKKRKV (SEQ ID NO: 13) or PKKKRRV (SEQ ID NO: 14). The NLS may be a bipartite sequence, such as the NLS of nucleoplasmin, KRPAATKKAGQAKKKK (SEQ ID NO: 15). In a specific example, a single PKKKRKV (SEQ ID NO: 13) NLS may be linked at the C-terminus of the Cas protein. One or more linkers are optionally included at the fusion site.

Cas proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, e.g., WO 2014/089290 and WO 2013/176772, each of which is herein incorporated by reference in its entirety for all purposes. The cell-penetrating domain can be located at the N-terminus, the C-terminus, or anywhere within the Cas protein.

Cas proteins can also be operably linked to a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreen1), yellow fluorescent proteins (e.g., YFP, eYFP, Citrine, Venus, YPet, PhiYFP, ZsYellow1), blue fluorescent proteins (e.g., eBFP, eBFP2, Azurite, mKalamal, GFPuv, Sapphire, T-sapphire), cyan fluorescent proteins (e.g., eCFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, S1, T7, V5, VSV-G, histidine (His), biotin carboxyl carrier protein (BCCP), and calmodulin.

Cas proteins can also be tethered to labeled nucleic acids. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification), or can be achieved through one or more intervening linkers or adapter molecules such as streptavidin or aptamers. See, e.g., Pierce et al. (2005) Mini Rev. Med. Chem. 5(1):41-55; Duckworth et al. (2007) Angew. Chem. Int. Ed. Engl. 46(46):8819-8822; Schaeffer and Dixon (2009) Australian J. Chem. 62(10):1328-1332; Goodman et al. (2009) Chembiochem. 10(9):1551-1557; and Khatwani et al. (2012) Bioorg. Med. Chem. 20(14):4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid can be tethered to the C-terminus, the N-terminus, or to an internal region within the Cas protein. In one example, the labeled nucleic acid is tethered to the C-terminus or the N-terminus of the Cas protein. Likewise, the Cas protein can be tethered to the 5′ end, the 3′ end, or to an internal region within the labeled nucleic acid. That is, the labeled nucleic acid can be tethered in any orientation and polarity. For example, the Cas protein can be tethered to the 5′ end or the 3′ end of the labeled nucleic acid.

Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism. For example, the nucleic acid encoding the Cas protein can be modified to substitute codons having a higher frequency of usage in a bacterial cell, a yeast cell, a human cell, a non-human cell, a mammalian cell, a rodent cell, a mouse cell, a rat cell, or any other host cell of interest, as compared to the naturally occurring polynucleotide sequence. When a nucleic acid encoding the Cas protein is introduced into the cell, the Cas protein can be transiently, conditionally, or constitutively expressed in the cell.

Nucleic acids encoding Cas proteins can be stably integrated in the genome of a cell and operably linked to a promoter active in the cell. Alternatively, nucleic acids encoding Cas proteins can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding a gRNA. Alternatively, it can be in a vector or plasmid that is separate from the vector comprising the DNA encoding the gRNA. Promoters that can be used in an expression construct include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allow for the generation of compact expression cassettes to facilitate delivery. In preferred embodiments, promotors are accepted by regulatory authorities for use in humans. In certain embodiments, promotors drive expression in a liver cell.

Different promoters can be used to drive Cas expression or Cas9 expression. In some methods, small promoters are used so that the Cas or Cas9 coding sequence can fit into an AAV construct. For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., AAV2-mediated delivery, AAV5-mediated delivery, AAV8-mediated delivery, or AAV7m8-mediated delivery). For example, the nuclease agent can be CRISPR/Cas9, and a Cas9 mRNA and a gRNA targeting an intron 1 of an endogenous human ALB locus can be delivered via LNP-mediated delivery or AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

Cas proteins provided as mRNAs can be modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. mRNA encoding Cas proteins can also be capped. The cap can be, for example, a cap 1 structure in which the +1 ribonucleotide is methylated at the 2′O position of the ribose. The capping can, for example, give superior activity in vivo (e.g., by mimicking a natural cap), can result in a natural structure that reduce stimulation of the innate immune system of the host (e.g., can reduce activation of pattern recognition receptors in the innate immune system). mRNA encoding Cas proteins can also be polyadenylated (to comprise a poly(A) tail). mRNA encoding Cas proteins can also be modified to include pseudouridine (e.g., can be fully substituted with pseudouridine). As another example, capped and polyadenylated Cas mRNA containing N1-methyl-pseudouridine can be used. mRNA encoding Cas proteins can also be modified to include N1-methyl-pseudouridine (e.g., can be fully substituted with N1-methyl-pseudouridine). As another example, Cas mRNA fully substituted with pseudouridine can be used (i.e., all standard uracil residues are replaced with pseudouridine, a uridine isomer in which the uracil is attached with a carbon-carbon bond rather than nitrogen-carbon). As another example, Cas mRNA fully substituted with N1-methyl-pseudouridine can be used (i.e., all standard uracil residues are replaced with N1-methyl-pseudouridine). Likewise, Cas mRNAs can be modified by depletion of uridine using synonymous codons. For example, capped and polyadenylated Cas mRNA fully substituted with pseudouridine can be used. For example, capped and polyadenylated Cas mRNA fully substituted with N1-methyl-pseudouridine can be used.

Cas mRNAs can comprise a modified uridine at least at one, a plurality of, or all uridine positions. The modified uridine can be a uridine modified at the 5 position (e.g., with a halogen, methyl, or ethyl). The modified uridine can be a pseudouridine modified at the 1 position (e.g., with a halogen, methyl, or ethyl). The modified uridine can be, for example, pseudouridine, N1-methyl-pseudouridine, 5-methoxyuridine, 5-iodouridine, or a combination thereof. In some examples, the modified uridine is 5-methoxyuridine. In some examples, the modified uridine is 5-iodouridine. In some examples, the modified uridine is pseudouridine. In some examples, the modified uridine is N1-methyl-pseudouridine. In some examples, the modified uridine is a combination of pseudouridine and N1-methyl-pseudouridine. In some examples, the modified uridine is a combination of pseudouridine and 5-methoxyuridine. In some examples, the modified uridine is a combination of N1-methyl pseudouridine and 5-methoxyuridine. In some examples, the modified uridine is a combination of 5-iodouridine and N1-methyl-pseudouridine. In some examples, the modified uridine is a combination of pseudouridine and 5-iodouridine. In some examples, the modified uridine is a combination of 5-iodouridine and 5-methoxyuridine.

Cas mRNAs disclosed herein can also comprise a 5′ cap, such as a Cap0, Cap1, or Cap2. A 5′ cap is generally a 7-methylguanine ribonucleotide (which may be further modified, e.g., with respect to ARCA) linked through a 5′-triphosphate to the 5′ position of the first nucleotide of the 5′-to-3′ chain of the mRNA (i.e., the first cap-proximal nucleotide). In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc. Natl. Acad. Sci. U.S.A. 111(33):12025-30 and Abbas et al. (2017) Proc. Natl. Acad. Sci. U.S.A. 114(11):E2106-E2115, each of which is herein incorporated by reference in its entirety for all purposes. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as non-self by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al. (2001) RNA 7:1486-1495, herein incorporated by reference in its entirety for all purposes.

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively.

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo and Moss (1990) Proc. Natl. Acad. Sci. U.S.A. 87:4023-4027 and Mao and Shuman (1994) J. Biol. Chem. 269:24472-24479, each of which is herein incorporated by reference in its entirety for all purposes.

Cas mRNAs can further comprise a poly-adenylated (poly-A or poly(A) or poly-adenine) tail. The poly-A tail can, for example, comprise at least 20, at least 30, at least 40, at least 50, at least 60, at least 70, at least 80, at least 90, or at least 100 adenines, and optionally up to 300 adenines. For example, the poly-A tail can comprise 95, 96, 97, 98, 99, or 100 adenine nucleotides.

### (3) Guide RNAs

A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” (also called “guide sequence”) and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. A guide RNA can refer to either a CRISPR RNA (crRNA) or the combination of a crRNA and a trans-activating CRISPR RNA (tracrRNA). The crRNA and tracrRNA can be associated as a single RNA molecule (single guide RNA or sgRNA) or in two separate RNA molecules (dual guide RNA or dgRNA). For Cas9, for example, a single-guide RNA can comprise a crRNA fused to a tracrRNA (e.g., via a linker). For Cpf1 and CasΦ, for example, only a crRNA is needed to achieve binding to a target sequence. The terms “guide RNA” and “gRNA” include both double-molecule (i.e., modular) gRNAs and single-molecule gRNAs. In some of the methods and compositions disclosed herein, a gRNA is a S. pyogenes Cas9 gRNA or an equivalent thereof. In some of the methods and compositions disclosed herein, a gRNA is a S. aureus Cas9 gRNA or an equivalent thereof.

An exemplary two-molecule gRNA comprises a crRNA-like (“CRISPR RNA” or “targeter-RNA” or “crRNA” or “crRNA repeat”) molecule and a corresponding tracrRNA-like (“trans-activating CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA. An example of a crRNA tail (e.g., for use with S. pyogenes Cas9), located downstream (3′) of the DNA-targeting segment, comprises, consists essentially of, or consists of GUUUUAGAGCUAUGCU (SEQ ID NO: 16) or GUUUUAGAGCUAUGCUGUUUUG (SEQ ID NO: 17). Any of the DNA-targeting segments disclosed herein can be joined to the 5′ end of SEQ ID NO: 16 or 17 to form a crRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. Examples of tracrRNA sequences (e.g., for use with S. pyogenes Cas9) comprise, consist essentially of, or consist of any one of

(SEQ ID NO: 18)
AGCAUAGCAAGUUAAAAUAAGGCUAGUCCGUUAUCAACUUGAAAAAGU
GGCACCGAGUCGGUGCUUU,
(SEQ ID NO: 19)
AAACAGCAUAGCAAGUUAAAAUAAGGCUAGUCCGUUAUCAACUUGAAA
AAGUGGCACCGAGUCGGUGCUUUU,
or
(SEQ ID NO: 20)
GUUGGAACCAUUCAAAACAGCAUAGCAAGUUAAAAUAAGGCUAGUCCG
UUAUCAACUUGAAAAAGUGGCACCGAGUCGGUGC.

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to the complementary strand of a target DNA. If used for modification within a cell, the exact sequence of a given crRNA or tracrRNA molecule can be designed to be specific to the species in which the RNA molecules will be used. See, e.g., Mali et al. (2013) Science 339(6121):823-826; Jinek et al. (2012) Science 337(6096):816-821; Hwang et al. (2013) Nat. Biotechnol. 31(3):227-229; Jiang et al. (2013) Nat. Biotechnol. 31(3):233-239; and Cong et al. (2013) Science 339(6121):819-823, each of which is herein incorporated by reference in its entirety for all purposes.

The DNA-targeting segment (crRNA) of a given gRNA comprises a nucleotide sequence that is complementary to a sequence on the complementary strand of the target DNA, as described in more detail below. The DNA-targeting segment of a gRNA interacts with the target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have, for example, a length of at least about 12, at least about 15, at least about 17, at least about 18, at least about 19, at least about 20, at least about 25, at least about 30, at least about 35, or at least about 40 nucleotides. Such DNA-targeting segments can have, for example, a length from about 12 to about 100, from about 12 to about 80, from about 12 to about 50, from about 12 to about 40, from about 12 to about 30, from about 12 to about 25, or from about 12 to about 20 nucleotides. For example, the DNA targeting segment can be from about 15 to about 25 nucleotides (e.g., from about 17 to about 20 nucleotides, or about 17, 18, 19, or 20 nucleotides). See, e.g., US 2016/0024523, herein incorporated by reference in its entirety for all purposes. For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpf1, a typical DNA-targeting segment is at least 16 nucleotides in length or at least 18 nucleotides in length.

In one example, the DNA-targeting segment can be about 20 nucleotides in length. However, shorter and longer sequences can also be used for the targeting segment (e.g., 15-25 nucleotides in length, such as 15, 16, 17, 18, 19, 20, 21, 22, 23, 24, or 25 nucleotides in length). The degree of identity between the DNA-targeting segment and the corresponding guide RNA target sequence (or degree of complementarity between the DNA-targeting segment and the other strand of the guide RNA target sequence) can be, for example, about 75%, about 80%, about 85%, about 90%, about 95%, or 100%. The DNA-targeting segment and the corresponding guide RNA target sequence can contain one or more mismatches. For example, the DNA-targeting segment of the guide RNA and the corresponding guide RNA target sequence can contain 1-4, 1-3, 1-2, 1, 2, 3, or 4 mismatches (e.g., where the total length of the guide RNA target sequence is at least 17, at least 18, at least 19, or at least 20 or more nucleotides). For example, the DNA-targeting segment of the guide RNA and the corresponding guide RNA target sequence can contain 1-4, 1-3, 1-2, 1, 2, 3, or 4 mismatches where the total length of the guide RNA target sequence 20 nucleotides.

As one example, a guide RNA targeting intron 1 of a human ALB gene can comprise a DNA-targeting segment (i.e., guide sequence) comprising, consisting essentially of, or consisting of the sequence (DNA-targeting segment) set forth in any one of SEQ ID NOS: 30-61. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 30-61. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in any one of SEQ ID NOS: 30-61. Alternatively, a guide RNA targeting intron 1 of a human ALB gene can comprise a DNA-targeting segment that is at least 90% or at least 95% identical to the sequence (DNA-targeting segment) set forth in any one of SEQ ID NOS: 30-61. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 30-61. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 30-61. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 30-61. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 30-61.

As another example, a guide RNA targeting intron 1 of a human ALB gene can comprise a DNA-targeting segment (i.e., guide sequence) comprising, consisting essentially of, or consisting of the sequence (DNA-targeting segment) set forth in any one of SEQ ID NOS: 36, 30, 33, and 41. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 36, 30, 33, and 41. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in any one of SEQ ID NOS: 36, 30, 33, and 41. Alternatively, a guide RNA targeting intron 1 of a human ALB gene can comprise a DNA-targeting segment that is at least 90% or at least 95% identical to the sequence (DNA-targeting segment) set forth in any one of SEQ ID NOS: 36, 30, 33, and 41. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 36, 30, 33, and 41. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 36, 30, 33, and 41. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 36, 30, 33, and 41. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 36, 30, 33, and 41.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 36. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 36. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in SEQ ID NO: 36. Alternatively, a guide RNA targeting intron 1 of a human ALB gene can comprise a DNA-targeting segment that is at least 90% or at least 95% identical to the sequence (DNA-targeting segment) set forth in SEQ ID NO: 36. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 36. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 36. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 36. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 36.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 30. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 30. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in SEQ ID NO: 30. Alternatively, a guide RNA targeting intron 1 of a human ALB gene can comprise a DNA-targeting segment that is at least 90% or at least 95% identical to the sequence (DNA-targeting segment) set forth in SEQ ID NO: 30. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 30. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 30. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 30. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 30.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 33. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 33. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in SEQ ID NO: 33. Alternatively, a guide RNA targeting intron 1 of a human ALB gene can comprise a DNA-targeting segment that is at least 90% or at least 95% identical to the sequence (DNA-targeting segment) set forth in SEQ ID NO: 33. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 33. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 33. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 33. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 33.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 41. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 41. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in SEQ ID NO: 41. Alternatively, a guide RNA targeting intron 1 of a human ALB gene can comprise a DNA-targeting segment that is at least 90% or at least 95% identical to the sequence (DNA-targeting segment) set forth in SEQ ID NO: 41. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 41. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 41. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 41. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 41.

TABLE 5
Human ALB Intron 1 Guide Sequences.

| Guide Sequence | SEQ ID NO: |
|---|---|
| GAGCAACCUCACUCUUGUCU | 30 |
| AUGCAUUUGUUUCAAAAUAU | 31 |
| UGCAUUUGUUUCAAAAUAUU | 32 |
| AUUUAUGAGAUCAACAGCAC | 33 |
| GAUCAACAGCACAGGUUUUG | 34 |
| UUAAAUAAAGCAUAGUGCAA | 35 |
| UAAAGCAUAGUGCAAUGGAU | 36 |
| UAGUGCAAUGGAUAGGUCUU | 37 |
| UACUAAAACUUUAUUUUACU | 38 |
| AAAGUUGAACAAUAGAAAAA | 39 |
| AAUGCAUAAUCUAAGUCAAA | 40 |
| UAAUAAAAUUCAAACAUCCU | 41 |
| GCAUCUUUAAAGAAUUAUUU | 42 |
| UUUGGCAUUUAUUUCUAAAA | 43 |
| UGUAUUUGUGAAGUCUUACA | 44 |
| UCCUAGGUAAAAAAAAAAAA | 45 |
| UAAUUUUCUUUUGCGCACUA | 46 |
| UGACUGAAACUUCACAGAAU | 47 |
| GACUGAAACUUCACAGAAUA | 48 |
| UUCAUUUUAGUCUGUCUUCU | 49 |
| AUUAUCUAAGUUUGAAUAUA | 50 |
| AAUUUUUAAAAUAGUAUUCU | 51 |
| UGAAUUAUUCUUCUGUUUAA | 52 |
| AUCAUCCUGAGUUUUUCUGU | 53 |
| UUACUAAAACUUUAUUUUAC | 54 |
| ACCUUUUUUUUUUUUUACCU | 55 |
| AGUGCAAUGGAUAGGUCUUU | 56 |
| UGAUUCCUACAGAAAAACUC | 57 |
| UGGGCAAGGGAAGAAAAAAA | 58 |
| CCUCACUCUUGUCUGGGCAA | 59 |
| ACCUCACUCUUGUCUGGGCA | 60 |
| UGAGCAACCUCACUCUUGUC | 61 |

TABLE 6
Human ALB Intron 1 sgRNA Sequences.

| Full Sequence | Full Sequence Modified |
|---|---|
| GAGCAACCUCACUCUUGUCUGUUU | mG*mA*mG*CAACCUCACUCUUGUCUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 62) | U*mU*mU (SEQ ID NO: 94) |
| AUGCAUUUGUUUCAAAAUAUGUUU | mA*mU*mG*CAUUUGUUUCAAAAUAUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 63) | U*mU*mU (SEQ ID NO: 95) |
| UGCAUUUGUUUCAAAAUAUUGUUU | mU*mG*mC*AUUUGUUUCAAAAUAUUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 64) | U*mU*mU (SEQ ID NO: 96) |
| AUUUAUGAGAUCAACAGCACGUUU | mA*mU*mU*UAUGAGAUCAACAGCACGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 65) | U*mU*mU (SEQ ID NO: 97) |
| GAUCAACAGCACAGGUUUUGGUUU | mG*mA*mU*CAACAGCACAGGUUUUGGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 66) | U*mU*mU (SEQ ID NO: 98) |
| UUAAAUAAAGCAUAGUGCAAGUUU | mU*mU*mA*AAUAAAGCAUAGUGCAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 67) | U*mU*mU (SEQ ID NO: 99) |
| UAAAGCAUAGUGCAAUGGAUGUUU | mU*mA*mA*AGCAUAGUGCAAUGGAUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 68) | U*mU*mU (SEQ ID NO: 100) |
| UAGUGCAAUGGAUAGGUCUUGUUU | mU*mA*mG*UGCAAUGGAUAGGUCUUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 69) | U*mU*mU (SEQ ID NO: 101) |
| UACUAAAACUUUAUUUUACUGUUU | mU*mA*mC*UAAAACUUUAUUUUACUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 70) | U*mU*mU (SEQ ID NO: 102) |
| AAAGUUGAACAAUAGAAAAAGUUU | mA*mA*mA*GUUGAACAAUAGAAAAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 71) | U*mU*mU (SEQ ID NO: 103) |
| AAUGCAUAAUCUAAGUCAAAGUUU | mA*mA*mU*GCAUAAUCUAAGUCAAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 72) | U*mU*mU (SEQ ID NO: 104) |
| UAAUAAAAUUCAAACAUCCUGUUU | mU*mA*mA*UAAAAUUCAAACAUCCUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 73) | U*mU*mU (SEQ ID NO: 105) |
| GCAUCUUUAAAGAAUUAUUUGUUU | mG*mC*mA*UCUUUAAAGAAUUAUUUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 74) | U*mU*mU (SEQ ID NO: 106) |
| UUUGGCAUUUAUUUCUAAAAGUUU | mU*mU*mU*GGCAUUUAUUUCUAAAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 75) | U*mU*mU (SEQ ID NO: 107) |
| UGUAUUUGUGAAGUCUUACAGUUU | mU*mG*mU*AUUUGUGAAGUCUUACAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 76) | U*mU*mU (SEQ ID NO: 108) |
| UCCUAGGUAAAAAAAAAAAAGUUU | mU*mC*mC*UAGGUAAAAAAAAAAAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 77) | U*mU*mU (SEQ ID NO: 109) |
| UAAUUUUCUUUUGCGCACUAGUUU | mU*mA*mA*UUUUCUUUUGCGCACUAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 78) | U*mU*mU (SEQ ID NO: 110) |
| UGACUGAAACUUCACAGAAUGUUU | mU*mG*mA*CUGAAACUUCACAGAAUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 79) | U*mU*mU (SEQ ID NO: 111) |
| GACUGAAACUUCACAGAAUAGUUU | mG*mA*mC*UGAAACUUCACAGAAUAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 80) | U*mU*mU (SEQ ID NO: 112) |
| UUCAUUUUAGUCUGUCUUCUGUUU | mU*mU*mC*AUUUUAGUCUGUCUUCUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 81) | U*mU*mU (SEQ ID NO: 113) |
| AUUAUCUAAGUUUGAAUAUAGUUU | mA*mU*mU*AUCUAAGUUUGAAUAUAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 82) | U*mU*mU (SEQ ID NO: 114) |
| AAUUUUUAAAAUAGUAUUCUGUUU | mA*mA*mU*UUUUAAAAUAGUAUUCUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 83) | U*mU*mU (SEQ ID NO: 115) |
| UGAAUUAUUCUUCUGUUUAAGUUU | mU*mG*mA*AUUAUUCUUCUGUUUAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 84) | U*mU*mU (SEQ ID NO: 116) |
| AUCAUCCUGAGUUUUUCUGUGUUU | mA*mU*mC*AUCCUGAGUUUUUCUGUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 85) | U*mU*mU (SEQ ID NO: 117) |
| UUACUAAAACUUUAUUUUACGUUU | mU*mU*mA*CUAAAACUUUAUUUUACGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 86) | U*mU*mU (SEQ ID NO: 118) |
| ACCUUUUUUUUUUUUUACCUGUUU | mA*mC*mC*UUUUUUUUUUUUUACCUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 87) | U*mU*mU (SEQ ID NO: 119) |
| AGUGCAAUGGAUAGGUCUUUGUUU | mA*mG*mU*GCAAUGGAUAGGUCUUUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 88) | U*mU*mU (SEQ ID NO: 120) |
| UGAUUCCUACAGAAAAACUCGUUU | mU*mG*mA*UUCCUACAGAAAAACUCGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 89) | U*mU*mU (SEQ ID NO: 121) |
| UGGGCAAGGGAAGAAAAAAAGUUU | mU*mG*mG*GCAAGGGAAGAAAAAAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 90) | U*mU*mU (SEQ ID NO: 122) |
| CCUCACUCUUGUCUGGGCAAGUUU | mC*mC*mU*CACUCUUGUCUGGGCAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 91) | U*mU*mU (SEQ ID NO: 123) |
| ACCUCACUCUUGUCUGGGCAGUUU | mA*mC*mC*UCACUCUUGUCUGGGCAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 92) | U*mU*mU (SEQ ID NO: 124) |
| UGAGCAACCUCACUCUUGUCGUUU | mU*mG*mA*GCAACCUCACUCUUGUCGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 93) | U*mU*mU (SEQ ID NO: 125) |

TABLE 7
Mouse Alb Intron 1 Guide Sequences.

| Guide Sequence | SEQ ID NO: |
|---|---|
| CACUCUUGUCUGUGGAAACA | 164 |

TABLE 8
Mouse Alb Intron 1 sgRNA Sequences.

| Full Sequence | Full Sequence Modified |
|---|---|
| CACUCUUGUCUGUGGAAACAGUUU | mC*mA*mC*UCUUGUCUGUGGA |
| UAGAGCUAGAAAUAGCAAGUUAAA | AACAGUUUUAGAmGmCmUmAmG |
| AUAAGGCUAGUCCGUUAUCAACUU | mAmAmAmUmAmGmCAAGUUAAA |
| GAAAAAGUGGCACCGAGUCGGUGC | AUAAGGCUAGUCCGUUAUCAmA |
| UUUU (SEQ ID NO: 166) | mCmUmUmGmAmAmAmAmAmGmU |
| | mGmGmCmAmCmCmGmAmGmUmC |
| | mGmGmUmGmCmU*mU*mU*mU |
| | (SEQ ID NO: 167) |

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise, consist essentially of, or consist of all or a portion of a wild type tracrRNA sequence (e.g., about or more than about 20, 26, 32, 45, 48, 54, 63, 67, 85, or more nucleotides of a wild type tracrRNA sequence). Examples of wild type tracrRNA sequences from S. pyogenes include 171-nucleotide, 89-nucleotide, 75-nucleotide, and 65-nucleotide versions. See, e.g., Deltcheva et al. (2011) Nature 471(7340):602-607; WO 2014/093661, each of which is herein incorporated by reference in its entirety for all purposes. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA. See U.S. Pat. No. 8,697,359, herein incorporated by reference in its entirety for all purposes.

The percent complementarity between the DNA-targeting segment of the guide RNA and the complementary strand of the target DNA can be at least 60% (e.g., at least 65%, at least 70%, at least 75%, at least 80%, at least 85%, at least 90%, at least 95%, or 100%). The percent complementarity between the DNA-targeting segment and the complementary strand of the target DNA can be at least 60% over about 20 contiguous nucleotides. As an example, the percent complementarity between the DNA-targeting segment and the complementary strand of the target DNA can be 100% over the 14 contiguous nucleotides at the 5′ end of the complementary strand of the target DNA and as low as 0% over the remainder. In such a case, the DNA-targeting segment can be considered to be 14 nucleotides in length. As another example, the percent complementarity between the DNA-targeting segment and the complementary strand of the target DNA can be 100% over the seven contiguous nucleotides at the 5′ end of the complementary strand of the target DNA and as low as 0% over the remainder. In such a case, the DNA-targeting segment can be considered to be 7 nucleotides in length. In some guide RNAs, at least 17 nucleotides within the DNA-targeting segment are complementary to the complementary strand of the target DNA. For example, the DNA-targeting segment can be 20 nucleotides in length and can comprise 1, 2, or 3 mismatches with the complementary strand of the target DNA. In one example, the mismatches are not adjacent to the region of the complementary strand corresponding to the protospacer adjacent motif (PAM) sequence (i.e., the reverse complement of the PAM sequence) (e.g., the mismatches are in the 5′ end of the DNA-targeting segment of the guide RNA, or the mismatches are at least 2, 3, 4, 5, 6, 7, 8, 9, 10, 11, 12, 13, 14, 15, 16, 17, 18, or 19 base pairs away from the region of the complementary strand corresponding to the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Single-guide RNAs can comprise a DNA-targeting segment and a scaffold sequence (i.e., the protein-binding or Cas-binding sequence of the guide RNA). For example, such guide RNAs can have a 5′ DNA-targeting segment joined to a 3′ scaffold sequence. Exemplary scaffold sequences (e.g., for use with S. pyogenes Cas9) comprise, consist essentially of, or consist of: GUUUUAGAGCUAGAAAUAGCAAGUUAAAAUAAGGCUAGUCCGUUAUCAACUUGA AAAAGUGGCACCGAGUCGGUGCU (version 1; SEQ ID NO: 21); GUUGGAACCAUUCAAAACAGCAUAGCAAGUUAAAAUAAGGCUAGUCCGUUAUCA ACUUGAAAAAGUGGCACCGAGUCGGUGC (version 2; SEQ ID NO: 22); GUUUUAGAGCUAGAAAUAGCAAGUUAAAAUAAGGCUAGUCCGUUAUCAACUUGA AAAAGUGGCACCGAGUCGGUGC (version 3; SEQ ID NO: 23); and GUUUAAGAGCUAUGCUGGAAACAGCAUAGCAAGUUUAAAUAAGGCUAGUCCGUU AUCAACUUGAAAAAGUGGCACCGAGUCGGUGC (version 4; SEQ ID NO: 24); GUUUUAGAGCUAGAAAUAGCAAGUUAAAAUAAGGCUAGUCCGUUAUCAACUUGA AAAAGUGGCACCGAGUCGGUGCUUUUUUU (version 5; SEQ ID NO: 25); GUUUUAGAGCUAGAAAUAGCAAGUUAAAAUAAGGCUAGUCCGUUAUCAACUUGA AAAAGUGGCACCGAGUCGGUGCUUUU (version 6; SEQ ID NO: 26); GUUUAAGAGCUAUGCUGGAAACAGCAUAGCAAGUUUAAAUAAGGCUAGUCCGUU AUCAACUUGAAAAAGUGGCACCGAGUCGGUGCUUUUUU (version 7; SEQ ID NO: 27); or GUUUUAGAGCUAGAAAUAGCAAGUUAAAAUAAGGCUAGUCCGUUAUCAACUUGG CACCGAGUCGGUGC (version 8; SEQ ID NO: 28). In some guide sgRNAs, the four terminal U residues of version 6 are not present. In some sgRNAs, only 1, 2, or 3 of the four terminal U residues of version 6 are present. Guide RNAs targeting any of the guide RNA target sequences disclosed herein can include, for example, a DNA-targeting segment on the 5′ end of the guide RNA fused to any of the exemplary guide RNA scaffold sequences on the 3′ end of the guide RNA. That is, any of the DNA-targeting segments disclosed herein can be joined to the 5′ end of any one of the above scaffold sequences to form a single guide RNA (chimeric guide RNA).

Guide RNAs can include modifications or sequences that provide for additional desirable features (e.g., modified or regulated stability; subcellular targeting; tracking with a fluorescent label; a binding site for a protein or protein complex; and the like). That is, guide RNAs can include one or more modified nucleosides or nucleotides, or one or more non-naturally and/or naturally occurring components or configurations that are used instead of or in addition to the canonical A, G, C, and U residues. Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof. Other examples of modifications include engineered stem loop duplex structures, engineered bulge regions, engineered hairpins 3′ of the stem loop duplex structure, or any combination thereof. See, e.g., US 2015/0376586, herein incorporated by reference in its entirety for all purposes. A bulge can be an unpaired region of nucleotides within the duplex made up of the crRNA-like region and the minimum tracrRNA-like region. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

Guide RNAs can comprise modified nucleosides and modified nucleotides including, for example, one or more of the following: (1) alteration or replacement of one or both of the non-linking phosphate oxygens and/or of one or more of the linking phosphate oxygens in the phosphodiester backbone linkage (an exemplary backbone modification); (2) alteration or replacement of a constituent of the ribose sugar such as alteration or replacement of the 2′ hydroxyl on the ribose sugar (an exemplary sugar modification); (3) replacement (e.g., wholesale replacement) of the phosphate moiety with dephospho linkers (an exemplary backbone modification); (4) modification or replacement of a naturally occurring nucleobase, including with a non-canonical nucleobase (an exemplary base modification); (5) replacement or modification of the ribose-phosphate backbone (an exemplary backbone modification); (6) modification of the 3′ end or 5′ end of the oligonucleotide (e.g., removal, modification or replacement of a terminal phosphate group or conjugation of a moiety, cap, or linker (such 3′ or 5′ cap modifications may comprise a sugar and/or backbone modification); and (7) modification or replacement of the sugar (an exemplary sugar modification). Other possible guide RNA modifications include modifications of or replacement of uracils or poly-uracil tracts. See, e.g., WO 2015/048577 and US 2016/0237455, each of which is herein incorporated by reference in its entirety for all purposes. Similar modifications can be made to Cas-encoding nucleic acids, such as Cas mRNAs. For example, Cas mRNAs can be modified by depletion of uridine using synonymous codons.

Chemical modifications such at hose listed above can be combined to provide modified gRNAs and/or mRNAs comprising residues (nucleosides and nucleotides) that can have two, three, four, or more modifications. For example, a modified residue can have a modified sugar and a modified nucleobase. In one example, every base of a gRNA is modified (e.g., all bases have a modified phosphate group, such as a phosphorothioate group). For example, all or substantially all of the phosphate groups of a gRNA can be replaced with phosphorothioate groups. Alternatively, or additionally, a modified gRNA can comprise at least one modified residue at or near the 5′ end. Alternatively, or additionally, a modified gRNA can comprise at least one modified residue at or near the 3′ end.

Some gRNAs comprise one, two, three or more modified residues. For example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the positions in a modified gRNA can be modified nucleosides or nucleotides.

Unmodified nucleic acids can be prone to degradation. Exogenous nucleic acids can also induce an innate immune response. Modifications can help introduce stability and reduce immunogenicity. Some gRNAs described herein can contain one or more modified nucleosides or nucleotides to introduce stability toward intracellular or serum-based nucleases. Some modified gRNAs described herein can exhibit a reduced innate immune response when introduced into a population of cells.

The gRNAs disclosed herein can comprise a backbone modification in which the phosphate group of a modified residue can be modified by replacing one or more of the oxygens with a different substituent. The modification can include the wholesale replacement of an unmodified phosphate moiety with a modified phosphate group as described herein. Backbone modifications of the phosphate backbone can also include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (Rp) or the “S” configuration (Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group (a sugar modification). For example, the 2′ hydroxyl group (OH) can be modified (e.g., replaced with a number of different oxy or deoxy substituents. Modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O),CH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). The 2′ hydroxyl group modification can be 2′-O-Me. Likewise, the 2′ hydroxyl group modification can be a 2′-fluoro modification, which replaces the 2′ hydroxyl group with a fluoride. The 2′ hydroxyl group modification can include locked nucleic acids (LNA) in which the 2′ hydroxyl can be connected, e.g., by a C1-6 alkylene or C1.6 heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)n-amino, (wherein amino can be, e.g., NH₂; alkylamino, dialkylamino, heterocyclyl, arylamino, diarylamino, heteroarylamino, or diheteroarylamino, ethylenediamine, or polyamino). The 2′ hydroxyl group modification can include unlocked nucleic acids (UNA) in which the ribose ring lacks the C2′—C3′ bond. The 2′ hydroxyl group modification can include the methoxyethyl group (MOE), (OCH₂CH₂OCH₃, e.g., a PEG derivative).

Deoxy 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH2; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form (e.g., L-nucleosides).

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In a dual guide RNA, each of the crRNA and the tracrRNA can contain modifications. Such modifications may be at one or both ends of the crRNA and/or tracrRNA. In a sgRNA, one or more residues at one or both ends of the sgRNA may be chemically modified, and/or internal nucleosides may be modified, and/or the entire sgRNA may be chemically modified. Some gRNAs comprise a 5′ end modification. Some gRNAs comprise a 3′ end modification.

The guide RNAs disclosed herein can comprise one of the modification patterns disclosed in WO 2018/107028 A1, herein incorporated by reference in its entirety for all purposes. The guide RNAs disclosed herein can also comprise one of the structures/modification patterns disclosed in US 2017/0114334, herein incorporated by reference in its entirety for all purposes. The guide RNAs disclosed herein can also comprise one of the structures/modification patterns disclosed in WO 2017/136794, WO 2017/004279, US 2018/0187186, or US 2019/0048338, each of which is herein incorporated by reference in its entirety for all purposes.

As one example, nucleotides at the 5′ or 3′ end of a guide RNA can include phosphorothioate linkages (e.g., the bases can have a modified phosphate group that is a phosphorothioate group). For example, a guide RNA can include phosphorothioate linkages between the 2, 3, or 4 terminal nucleotides at the 5′ or 3′ end of the guide RNA. As another example, nucleotides at the 5′ and/or 3′ end of a guide RNA can have 2′-O-methyl modifications. For example, a guide RNA can include 2′-O-methyl modifications at the 2, 3, or 4 terminal nucleotides at the 5′ and/or 3′ end of the guide RNA (e.g., the 5′ end). See, e.g., WO 2017/173054 A1 and Finn et al. (2018) Cell Rep. 22(9):2227-2235, each of which is herein incorporated by reference in its entirety for all purposes. Other possible modifications are described in more detail elsewhere herein. In a specific example, a guide RNA includes 2′-O-methyl analogs and 3′ phosphorothioate internucleotide linkages at the first three 5′ and 3′ terminal RNA residues. Such chemical modifications can, for example, provide greater stability and protection from exonucleases to guide RNAs, allowing them to persist within cells for longer than unmodified guide RNAs. Such chemical modifications can also, for example, protect against innate intracellular immune responses that can actively degrade RNA or trigger immune cascades that lead to cell death.

As one example, any of the guide RNAs described herein can comprise at least one modification. In one example, the at least one modification comprises a 2′-O-methyl (2′-O-Me) modified nucleotide, a phosphorothioate (PS) bond between nucleotides, a 2′-fluoro (2′-F) modified nucleotide, or a combination thereof. For example, the at least one modification can comprise a 2′-O-methyl (2′-O-Me) modified nucleotide. Alternatively, or additionally, the at least one modification can comprise a phosphorothioate (PS) bond between nucleotides. Alternatively, or additionally, the at least one modification can comprise a 2′-fluoro (2′-F) modified nucleotide. In one example, a guide RNA described herein comprises one or more 2′-O-methyl (2′-O-Me) modified nucleotides and one or more phosphorothioate (PS) bonds between nucleotides.

The modifications can occur anywhere in the guide RNA. As one example, the guide RNA comprises a modification at one or more of the first five nucleotides at the 5′ end of the guide RNA, the guide RNA comprises a modification at one or more of the last five nucleotides of the 3′ end of the guide RNA, or a combination thereof. For example, the guide RNA can comprise phosphorothioate bonds between the first four nucleotides of the guide RNA, phosphorothioate bonds between the last four nucleotides of the guide RNA, or a combination thereof. Alternatively, or additionally, the guide RNA can comprise 2′-O-Me modified nucleotides at the first three nucleotides at the 5′ end of the guide RNA, can comprise 2′-O-Me modified nucleotides at the last three nucleotides at the 3′ end of the guide RNA, or a combination thereof.

In one example, a modified gRNA can comprise the following sequence: mN*mN*mN*NNNNNNNNNNNNNNNNNGUUUUAGAmGmCmUmAmGmAmAmAmUmA mGmCAAGUUAAAAUAAGGCUAGUCCGUUAUCAmAmCmUmUmGmAmAmAmAmAm GmUmGmGmCmAmCmCmGmAmGmUmCmGmGmUmGmCmU*mU*mU*mU (SEQ ID NO: 29), where “N” may be any natural or non-natural nucleotide. For example, the totality of N residues comprise a human ALB intron 1 DNA-targeting segment as described herein (e.g., the sequence set forth in SEQ ID NO: 29, wherein the N residues are replaced with the DNA-targeting segment of any one of SEQ ID NOS: 30-61, the DNA-targeting segment of any one of SEQ ID NOS: 36, 30, 33, and 41, or the DNA-targeting segment of SEQ ID NO: 36. For example, a modified gRNA can comprise the sequence set forth in any one of SEQ ID NOS: 94-125, the sequence set forth in any one of SEQ ID NOS: 100, 94, 97, and 105, or the sequence set forth in SEQ ID NO: 100 in Table 6. The terms “mA,” “mC,” “mU,” and “mG” denote a nucleotide (A, C, U, and G, respectively) that has been modified with 2′-O-Me. The symbol “*” depicts a phosphorothioate modification. In certain embodiments, A, C, G, U, and N independently denote a ribose sugar, i.e., 2′-OH. In certain embodiments in the context of a modified sequence, A, C, G, U, and N denote a ribose sugar, i.e., 2′-OH. A phosphorothioate linkage or bond refers to a bond where a sulfur is substituted for one nonbridging phosphate oxygen in a phosphodiester linkage, for example, in the bonds between nucleotides bases. When phosphorothioates are used to generate oligonucleotides, the modified oligonucleotides may also be referred to as S-oligos. The terms A*, C*, U*, or G* denote a nucleotide that is linked to the next (e.g., 3′) nucleotide with a phosphorothioate bond. The terms “mA*,” “mC*,” “mU*,” and “mG*” denote a nucleotide (A, C, U, and G, respectively) that has been substituted with 2′-O-Me and that is linked to the next (e.g., 3′) nucleotide with a phosphorothioate bond.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Abasic nucleotides refer to those which lack nitrogenous bases. Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In one example, one or more of the first three, four, or five nucleotides at the 5′ terminus, and one or more of the last three, four, or five nucleotides at the 3′ terminus are modified. The modification can be, for example, a 2′-O-Me, 2′-F, inverted abasic nucleotide, phosphorothioate bond, or other nucleotide modification well known to increase stability and/or performance.

In another example, the first four nucleotides at the 5′ terminus, and the last four nucleotides at the 3′ terminus can be linked with phosphorothioate bonds.

In another example, the first three nucleotides at the 5′ terminus, and the last three nucleotides at the 3′ terminus can comprise a 2′-O-methyl (2′-O-Me) modified nucleotide. In another example, the first three nucleotides at the 5′ terminus, and the last three nucleotides at the 3′ terminus comprise a 2′-fluoro (2′-F) modified nucleotide. In another example, the first three nucleotides at the 5′ terminus, and the last three nucleotides at the 3′ terminus comprise an inverted abasic nucleotide.

Guide RNAs can be provided in any form. For example, the gRNA can be provided in the form of RNA, either as two molecules (separate crRNA and tracrRNA) or as one molecule (sgRNA), and optionally in the form of a complex with a Cas protein. The gRNA can also be provided in the form of DNA encoding the gRNA. The DNA encoding the gRNA can encode a single RNA molecule (sgRNA) or separate RNA molecules (e.g., separate crRNA and tracrRNA). In the latter case, the DNA encoding the gRNA can be provided as one DNA molecule or as separate DNA molecules encoding the crRNA and tracrRNA, respectively.

When a gRNA is provided in the form of DNA, the gRNA can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in a vector comprising a heterologous nucleic acid, such as a nucleic acid encoding a Cas protein. Alternatively, it can be in a vector or a plasmid that is separate from the vector comprising the nucleic acid encoding the Cas protein. Promoters that can be used in such expression constructs include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Such promoters can also be, for example, bidirectional promoters. Specific examples of suitable promoters include an RNA polymerase III promoter, such as a human U6 promoter, a rat U6 polymerase III promoter, or a mouse U6 polymerase III promoter.

Alternatively, gRNAs can be prepared by various other methods. For example, gRNAs can be prepared by in vitro transcription using, for example, T7 RNA polymerase (see, e.g., WO 2014/089290 and WO 2014/065596, each of which is herein incorporated by reference in its entirety for all purposes). Guide RNAs can also be a synthetically produced molecule prepared by chemical synthesis. For example, a guide RNA can be chemically synthesized to include 2′-O-methyl analogs and 3′ phosphorothioate internucleotide linkages at the first three 5′ and 3′ terminal RNA residues.

Guide RNAs (or nucleic acids encoding guide RNAs) can be in compositions comprising one or more guide RNAs (e.g., 1, 2, 3, 4, or more guide RNAs) and a carrier increasing the stability of the guide RNA (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules. Such compositions can further comprise a Cas protein, such as a Cas9 protein, or a nucleic acid encoding a Cas protein.

As one example, a guide RNA targeting intron 1 of a human ALB gene can comprise, consist essentially of, or consist of the sequence set forth in any one of SEQ ID NOS: 62-125. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in any one of SEQ ID NOS: 62-125. Alternatively, a guide RNA targeting intron 1 of a human ALB gene can comprise, consist essentially of, or consist of a sequence that is at least 90% or at least 95% identical to the DNA-targeting segment set forth in any one of SEQ ID NOS: 62-125. Alternatively, a guide RNA targeting intron 1 of a human ALB gene can comprise, consist essentially of, or consist of a sequence that differs by no more than 3, no more than 2, or no more than 1 nucleotide from the sequence set forth in any one of SEQ ID NOS: 62-125.

As another example, a guide RNA targeting intron 1 of a human ALB gene can comprise, consist essentially of, or consist of the sequence set forth in any one of SEQ ID NOS: 68, 100, 62, 94, 65, 97, 73, and 105. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in any one of SEQ ID NOS: 68, 100, 62, 94, 65, 97, 73, and 105. Alternatively, a guide RNA targeting intron 1 of a human ALB gene can comprise, consist essentially of, or consist of a sequence that is at least 90% or at least 95% identical to the DNA-targeting segment set forth in any one of SEQ ID NOS: 68, 100, 62, 94, 65, 97, 73, and 105. Alternatively, a guide RNA targeting intron 1 of a human ALB gene can comprise, consist essentially of, or consist of a sequence that differs by no more than 3, no more than 2, or no more than 1 nucleotide from the sequence set forth in any one of SEQ ID NOS: 68, 100, 62, 94, 65, 97, 73, and 105.

As another example, a guide RNA targeting intron 1 of a human ALB gene can comprise, consist essentially of, or consist of the sequence set forth in SEQ ID NO: 68 or 100. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in SEQ ID NO: 68 or 100. Alternatively, a guide RNA targeting intron 1 of a human ALB gene can comprise, consist essentially of, or consist of a sequence that is at least 90% or at least 95% identical to the DNA-targeting segment set forth in SEQ ID NO: 68 or 100. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 68 or 100.

As another example, a guide RNA targeting intron 1 of a human ALB gene can comprise, consist essentially of, or consist of the sequence set forth in SEQ ID NO: 62 or 94. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in SEQ ID NO: 62 or 94. Alternatively, a guide RNA targeting intron 1 of a human ALB gene can comprise, consist essentially of, or consist of a sequence that is at least 90% or at least 95% identical to the DNA-targeting segment set forth in SEQ ID NO: 62 or 94. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 62 or 94.

As another example, a guide RNA targeting intron 1 of a human ALB gene can comprise, consist essentially of, or consist of the sequence set forth in SEQ ID NO: 65 or 97. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in SEQ ID NO: 65 or 97. Alternatively, a guide RNA targeting intron 1 of a human ALB gene can comprise, consist essentially of, or consist of a sequence that is at least 90% or at least 95% identical to the DNA-targeting segment set forth in SEQ ID NO: 65 or 97. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 65 or 97.

As another example, a guide RNA targeting intron 1 of a human ALB gene can comprise, consist essentially of, or consist of the sequence set forth in SEQ ID NO: 73 or 105. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in SEQ ID NO: 73 or 105. Alternatively, a guide RNA targeting intron 1 of a human ALB gene can comprise, consist essentially of, or consist of a sequence that is at least 90% or at least 95% identical to the DNA-targeting segment set forth in SEQ ID NO: 73 or 105. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 73 or 105.

### (4) Guide RNA Target Sequences

Target DNAs for guide RNAs include nucleic acid sequences present in a DNA to which a DNA-targeting segment of a gRNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001), herein incorporated by reference in its entirety for all purposes). The strand of the target DNA that is complementary to and hybridizes with the gRNA can be called the “complementary strand,” and the strand of the target DNA that is complementary to the “complementary strand” (and is therefore not complementary to the Cas protein or gRNA) can be called “noncomplementary strand” or “template strand.”

The target DNA includes both the sequence on the complementary strand to which the guide RNA hybridizes and the corresponding sequence on the non-complementary strand (e.g., adjacent to the protospacer adjacent motif (PAM)). The term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand. A guide RNA is designed to have complementarity to the complementary strand of a target DNA, where hybridization between the DNA-targeting segment of the guide RNA and the complementary strand of the target DNA promotes the formation of a CRISPR complex. Full complementarity is not necessarily required, provided that there is sufficient complementarity to cause hybridization and promote formation of a CRISPR complex. If a guide RNA is referred to herein as targeting a guide RNA target sequence, what is meant is that the guide RNA hybridizes to the complementary strand sequence of the target DNA that is the reverse complement of the guide RNA target sequence on the non-complementary strand.

A target DNA or guide RNA target sequence can comprise any polynucleotide, and can be located, for example, in the nucleus or cytoplasm of a cell or within an organelle of a cell, such as a mitochondrion or chloroplast. A target DNA or guide RNA target sequence can be any nucleic acid sequence endogenous or exogenous to a cell. The guide RNA target sequence can be a sequence coding a gene product (e.g., a protein) or a non-coding sequence (e.g., a regulatory sequence) or can include both.

Site-specific binding and cleavage of a target DNA by a Cas protein can occur at locations determined by both (i) base-pairing complementarity between the guide RNA and the complementary strand of the target DNA and (ii) a short motif, called the protospacer adjacent motif (PAM), in the non-complementary strand of the target DNA. The PAM can flank the guide RNA target sequence. Optionally, the guide RNA target sequence can be flanked on the 3′ end by the PAM (e.g., for Cas9). Alternatively, the guide RNA target sequence can be flanked on the 5′ end by the PAM (e.g., for Cpf1). For example, the cleavage site of Cas proteins can be about 1 to about 10 or about 2 to about 5 base pairs (e.g., 3 base pairs) upstream or downstream of the PAM sequence (e.g., within the guide RNA target sequence). In the case of SpCas9, the PAM sequence (i.e., on the non-complementary strand) can be 5′-N₁GG-3′, where N₁ is any DNA nucleotide, and where the PAM is immediately 3′ of the guide RNA target sequence on the non-complementary strand of the target DNA. As such, the sequence corresponding to the PAM on the complementary strand (i.e., the reverse complement) would be 5′-CCN₂-3′, where N₂ is any DNA nucleotide and is immediately 5′ of the sequence to which the DNA-targeting segment of the guide RNA hybridizes on the complementary strand of the target DNA. In some such cases, N₁ and N₂ can be complementary and the N₁—N₂ base pair can be any base pair (e.g., N₁=C and N₂=G; N₁=G and N₂=C; N₁=A and N₂=T; or N₁=T, and N₂=A). In the case of Cas9 from S. aureus, the PAM can be NNGRRT or NNGRR, where N can A, G, C, or T, and R can be G or A. In the case of Cas9 from C. jejuni, the PAM can be, for example, NNNNACAC or NNNNRYAC, where N can be A, G, C, or T, and R can be G or A. In some cases (e.g., for FnCpf1), the PAM sequence can be upstream of the 5′ end and have the sequence 5′-TTN-3′. In the case of DpbCasX, the PAM can have the sequence 5′-TTCN-3′. In the case of CasΦ, the PAM can have the sequence 5′-TBN-3′, wherein B is G, T, or C.

An example of a guide RNA target sequence is a 20-nucleotide DNA sequence immediately preceding an NGG motif recognized by an SpCas9 protein. For example, two examples of guide RNA target sequences plus PAMs are GN₁₉NGG (SEQ ID NO: 5) or N₂₀NGG (SEQ ID NO: 6). See, e.g., WO 2014/165825, herein incorporated by reference in its entirety for all purposes. The guanine at the 5′ end can facilitate transcription by RNA polymerase in cells. Other examples of guide RNA target sequences plus PAMs can include two guanine nucleotides at the 5′ end (e.g., GGN₂₀NGG; SEQ ID NO: 7) to facilitate efficient transcription by T7 polymerase in vitro. See, e.g., WO 2014/065596, herein incorporated by reference in its entirety for all purposes. Other guide RNA target sequences plus PAMs can have between 4-22 nucleotides in length of SEQ ID NOS: 5-7, including the 5′ G or GG and the 3′ GG or NGG. Yet other guide RNA target sequences plus PAMs can have between 14 and 20 nucleotides in length of SEQ ID NOS: 5-7.

Formation of a CRISPR complex hybridized to a target DNA can result in cleavage of one or both strands of the target DNA within or near the region corresponding to the guide RNA target sequence (i.e., the guide RNA target sequence on the non-complementary strand of the target DNA and the reverse complement on the complementary strand to which the guide RNA hybridizes). For example, the cleavage site can be within the guide RNA target sequence (e.g., at a defined location relative to the PAM sequence). The “cleavage site” includes the position of a target DNA at which a Cas protein produces a single-strand break or a double-strand break. The cleavage site can be on only one strand (e.g., when a nickase is used) or on both strands of a double-stranded DNA. Cleavage sites can be at the same position on both strands (producing blunt ends; e.g., Cas9)) or can be at different sites on each strand (producing staggered ends (i.e., overhangs); e.g., Cpf1). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA target sequence or cleavage site of the nickase on the first strand is separated from the guide RNA target sequence or cleavage site of the nickase on the second strand by at least 2, 3, 4, 5, 6, 7, 8, 9, 10, 15, 20, 25, 30, 40, 50, 75, 100, 250, 500, or 1,000 base pairs.

The guide RNA target sequence can also be selected to minimize off-target modification or avoid off-target effects (e.g., by avoiding two or fewer mismatches to off-target genomic sequences).

As one example, a guide RNA targeting intron 1 of a human ALB gene can target the guide RNA target sequence set forth in any one of SEQ ID NOS: 126-157. As another example, a guide RNA targeting intron 1 of a human ALB gene can target at least 17, at least 18, at least 19, or at least 20 contiguous nucleotides of the guide RNA target sequence set forth in any one of SEQ ID NOS: 126-157.

As another example, a guide RNA targeting intron 1 of a human ALB gene can target the guide RNA target sequence set forth in any one of SEQ ID NOS: 132, 126, 129, and 137. As another example, a guide RNA targeting intron 1 of a human ALB gene can target at least 17, at least 18, at least 19, or at least 20 contiguous nucleotides of the guide RNA target sequence set forth in any one of SEQ ID NOS: 132, 126, 129, and 137.

As another example, a guide RNA targeting intron 1 of a human ALB gene can target the guide RNA target sequence set forth in SEQ ID NO: 132. As another example, a guide RNA targeting intron 1 of a human ALB gene can target at least 17, at least 18, at least 19, or at least 20 contiguous nucleotides of the guide RNA target sequence set forth in SEQ ID NO: 132.

As another example, a guide RNA targeting intron 1 of a human ALB gene can target the guide RNA target sequence set forth in SEQ ID NO: 126. As another example, a guide RNA targeting intron 1 of a human ALB gene can target at least 17, at least 18, at least 19, or at least 20 contiguous nucleotides of the guide RNA target sequence set forth in SEQ ID NO: 126.

As another example, a guide RNA targeting intron 1 of a human ALB gene can target the guide RNA target sequence set forth in SEQ ID NO: 129. As another example, a guide RNA targeting intron 1 of a human ALB gene can target at least 17, at least 18, at least 19, or at least 20 contiguous nucleotides of the guide RNA target sequence set forth in SEQ ID NO: 129.

As another example, a guide RNA targeting intron 1 of a human ALB gene can target the guide RNA target sequence set forth in SEQ ID NO: 137. As another example, a guide RNA targeting intron 1 of a human ALB gene can target at least 17, at least 18, at least 19, or at least 20 contiguous nucleotides of the guide RNA target sequence set forth in SEQ ID NO: 137.

TABLE 9
Human ALB Intron 1 Guide RNA Target
Sequences.

| Guide RNA Target Sequence | SEQ ID NO: |
|---|---|
| GAGCAACCTCACTCTTGTCT | 126 |
| ATGCATTTGTTTCAAAATAT | 127 |
| TGCATTTGTTTCAAAATATT | 128 |
| ATTTATGAGATCAACAGCAC | 129 |
| GATCAACAGCACAGGTTTTG | 130 |
| TTAAATAAAGCATAGTGCAA | 131 |
| TAAAGCATAGTGCAATGGAT | 132 |
| TAGTGCAATGGATAGGTCTT | 133 |
| TACTAAAACTTTATTTTACT | 134 |
| AAAGTTGAACAATAGAAAAA | 135 |
| AATGCATAATCTAAGTCAAA | 136 |
| TAATAAAATTCAAACATCCT | 137 |
| GCATCTTTAAAGAATTATTT | 138 |
| TTTGGCATTTATTTCTAAAA | 139 |
| TGTATTTGTGAAGTCTTACA | 140 |
| TCCTAGGTAAAAAAAAAAAA | 141 |
| TAATTTTCTTTTGCGCACTA | 142 |
| TGACTGAAACTTCACAGAAT | 143 |
| GACTGAAACTTCACAGAATA | 144 |
| TTCATTTTAGTCTGTCTTCT | 145 |
| ATTATCTAAGTTTGAATATA | 146 |
| AATTTTTAAAATAGTATTCT | 147 |
| TGAATTATTCTTCTGTTTAA | 148 |
| ATCATCCTGAGTTTTTCTGT | 149 |
| TTACTAAAACTTTATTTTAC | 150 |
| ACCTTTTTTTTTTTTTACCT | 151 |
| AGTGCAATGGATAGGTCTTT | 152 |
| TGATTCCTACAGAAAAACTC | 153 |
| TGGGCAAGGGAAGAAAAAAA | 154 |
| CCTCACTCTTGTCTGGGCAA | 155 |
| ACCTCACTCTTGTCTGGGCA | 156 |
| TGAGCAACCTCACTCTTGTC | 157 |

TABLE 10
Mouse Alb Intron 1 Guide RNA Target
Sequences.

| Guide RNA Target Sequence | SEQ ID NO: |
|---|---|
| CACTCTTGTCTGTGGAAACA | 165 |

### (5) Lipid Nanoparticles Comprising Nuclease Agents

Lipid nanoparticles comprising the nuclease agents (e.g., CRISPR/Cas systems) are also provided. The lipid nanoparticles can alternatively or additionally comprise a nucleic acid construct encoding a multidomain therapeutic protein as disclosed herein. For example, the lipid nanoparticles can comprise a nuclease agent (e.g., CRISPR/Cas system), can comprise a nucleic acid construct encoding a multidomain therapeutic protein, or can comprise both a nuclease agent (e.g., a CRISPR/Cas system) and a nucleic acid construct encoding a multidomain therapeutic protein. Regarding CRISPR/Cas systems, the lipid nanoparticles can comprise the Cas protein in any form (e.g., protein, DNA, or mRNA) and/or can comprise the guide RNA(s) in any form (e.g., DNA or RNA). In one example, the lipid nanoparticles comprise the Cas protein in the form of mRNA (e.g., a modified RNA as described herein) and the guide RNA(s) in the form of RNA (e.g., a modified guide RNA as disclosed herein). As another example, the lipid nanoparticles can comprise the Cas protein in the form of protein and the guide RNA(s) in the form of RNA). In a specific example, the guide RNA and the Cas protein are each introduced in the form of RNA via LNP-mediated delivery in the same LNP. As discussed in more detail elsewhere herein, one or more of the RNAs can be modified. For example, guide RNAs can be modified to comprise one or more stabilizing end modifications at the 5′ end and/or the 3′ end. Such modifications can include, for example, one or more phosphorothioate linkages at the 5′ end and/or the 3′ end and/or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. As another example, Cas mRNA modifications can include substitution with N1-methyl-pseudouridine (e.g., fully substituted with N1-methyl-pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as distearoylphosphatidylcholine or 1,2-distearoyl-sn-glycero-3-phosphocholine (DSPC). In another example, the other components can comprise a helper lipid such as cholesterol, an optional neutral lipid such as DSPC, and a stealth lipid such as S010, 5024, 5027, 5031, or 5033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include a nucleic acid construct encoding a multidomain therapeutic protein as described elsewhere herein. In certain LNPs, the cargo can include an mRNA encoding a Cas nuclease, such as Cas9, a guide RNA or a nucleic acid encoding a guide RNA, and a nucleic acid construct encoding a multidomain therapeutic protein. In some LNPs, the lipid component comprises an amine lipid such as a biodegradable, ionizable lipid. In some instances, the lipid component comprises biodegradable, ionizable lipid, cholesterol, DSPC, and PEG-DMG. For example, Cas9 mRNA and gRNA can be delivered to cells and animals utilizing lipid formulations comprising ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z, 12Z)-octadeca-9,12-dienoate), cholesterol, DSPC, and PEG2k-DMG.

In some examples, the LNPs comprise cationic lipids. In some examples, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., WO 2019/067992, WO 2017/173054, WO 2015/095340, and WO 2014/136086, each of which is herein incorporated by reference in its entirety for all purposes. In some examples, the LNPs comprise molar ratios of a cationic lipid amine to RNA phosphate (N:P) of about 4.5, about 5.0, about 5.5, about 6.0, or about 6.5. In some examples, the terms cationic and ionizable in the context of LNP lipids are interchangeable (e.g., wherein ionizable lipids are cationic depending on the pH).

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235 and WO 2017/173054 A1, each of which is herein incorporated by reference in its entirety for all purposes. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Another example of a suitable lipid is Lipid C, which is 2-((4-(((3-(dimethylamino)propoxy)carbonyl)oxy)hexadecanoyl)oxy)propane-1,3-diyl(9Z,9′Z,12Z,12′Z)-bis(octadeca-9,12-dienoate). Another example of a suitable lipid is Lipid D, which is 3-(((3-(dimethylamino)propoxy)carbonyl)oxy)-13-(octanoyloxy)tridecyl 3-octylundecanoate. Other suitable lipids include heptatriaconta-6,9,28,31-tetraen-19-yl 4-(dimethylamino)butanoate (also known as [(6Z,9Z,28Z,31Z)-heptatriaconta-6,9,28,31-tetraen-19-yl]4-(dimethylamino)butanoate or Dlin-MC3-DMA (MC3))).

Some such lipids suitable for use in the LNPs described herein are biodegradable in vivo.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral lipids function to stabilize and improve processing of the LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine or 1,2-distearoyl-sn-glycero-3-phosphocholine (DSPC), phosphocholine (DOPC), dimyristoylphosphatidylcholine (DMPC), phosphatidylcholine (PLPC), 1,2-diarachidonoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, 1-stearoyl-2-oleoyl-sn-glycero-3-phosphocholine (SOPC), and combinations thereof. For example, the neutral phospholipid may be selected from the group consisting of distearoylphosphatidylcholine (DSPC) and dimyristoyl phosphatidyl ethanolamine (DMPE).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the LNP. Suitable stealth lipids include lipids having a hydrophilic head group linked to a lipid moiety.

The hydrophilic head group of a stealth lipid can comprise, for example, a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids, and poly N-(2-hydroxypropyl)methacrylamide. The term PEG means any polyethylene glycol or other polyalkylene ether polymer. In certain LNP formulations, the PEG, is a PEG-2K, also termed PEG 2000, which has an average molecular weight of about 2,000 daltons. See, e.g., WO 2017/173054 A1, herein incorporated by reference in its entirety for all purposes.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

As one example, the stealth lipid may be selected from PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMPE),or 1,2-dimyristoyl-rac-glycero-3-methylpolyoxyethylene glycol-2000 (PEG2k-DMG), 1,2-distearoyl-sn-glycero-3-phosphoethanolamine-N-[methoxy(polyethylene glycol)-2000] (PEG2k-DSPE), 1,2-distearoyl-sn-glycerol, methoxypoly ethylene glycol (PEG2k-DSG), poly(ethylene glycol)-2000-dimethacrylate (PEG2k-DMA), and 1,2-distearyloxypropyl-3-amine-N-[methoxy(polyethylene glycol)-2000] (PEG2k-DSA). In one particular example, the stealth lipid may be PEG2k-DMG.

In some embodiments, the PEG lipid includes a glycerol group. In some embodiments, the PEG lipid includes a dimyristoylglycerol (DMG) group. In some embodiments, the PEG lipid comprises PEG2k. In some embodiments, the PEG lipid is a PEG-DMG. In some embodiments, the PEG lipid is a PEG2k-DMG. In some embodiments, the PEG lipid is 1,2-dimyristoyl-rac-glycero-3-methoxypolyethylene glycol-2000. In some embodiments, the PEG2k-DMG is 1,2-dimyristoyl-rac-glycero-3-methoxypolyethylene glycol-2000.

The LNPs can comprise different respective molar ratios of the component lipids in the formulation. The mol-% of the CCD lipid may be, for example, from about 30 mol-% to about 60 mol-%. The mol-% of the helper lipid may be, for example, from about 30 mol-% to about 60 mol-%. The mol-% of the neutral lipid may be, for example, from about 1 mol-% to about 20 mol-%. The mol-% of the stealth lipid may be, for example, from about 1 mol-% to about 10 mol-%

The LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100. The N/P ratio can also be from about 4 to about 6.

In some LNPs, the cargo can comprise Cas mRNA (e.g., Cas9 mRNA) and gRNA. The Cas mRNA and gRNAs can be in different ratios. For example, the LNP formulation can include a ratio of Cas mRNA to gRNA nucleic acid ranging from about 25:1 to about 1:25. Alternatively, the LNP formulation can include a ratio of Cas mRNA to gRNA nucleic acid of from about 2:1 to about 1:2. In specific examples, the ratio of Cas mRNA to gRNA can be about 2:1.

In some LNPs, the cargo can comprise a nucleic acid construct encoding a multidomain therapeutic protein and gRNA. The nucleic acid construct encoding a multidomain therapeutic protein and gRNAs can be in different ratios. For example, the LNP formulation can include a ratio of nucleic acid construct to gRNA nucleic acid ranging from about 25:1 to about 1:25.

A specific example of a suitable LNP has a nitrogen-to-phosphate (N/P) ratio of about 4.5 and contains biodegradable cationic lipid, cholesterol, DSPC, and PEG2k-DMG in an about 45:44:9:2 molar ratio (about 45:about 44:about 9:about 2). The biodegradable cationic lipid can be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235, herein incorporated by reference in its entirety for all purposes. The Cas9 mRNA can be in an about 1:1 (about 1:about 1) ratio by weight to the guide RNA. Another specific example of a suitable LNP contains Dlin-MC3-DMA (MC3), cholesterol, DSPC, and PEG-DMG in an about 50:38.5:10:1.5 molar ratio (about 50:about 38.5:about 10:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP has a nitrogen-to-phosphate (N/P) ratio of about 6 and contains biodegradable cationic lipid, cholesterol, DSPC, and PEG2k-DMG in an about 50:38:9:3 molar ratio (about 50:about 38:about 9:about 3). The biodegradable cationic lipid can be Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 (about 2:about 1) ratio by weight to the guide RNA.

Another specific example of a suitable LNP has a nitrogen-to-phosphate (N/P) ratio of about 3 and contains a cationic lipid, a structural lipid, cholesterol (e.g., cholesterol (ovine) (Avanti 700000)), and PEG2k-DMG (e.g., PEG-DMG 2000 (NOF America-SUNBRIGHT® GM-020(DMG-PEG)) in an about 50:10:38.5:1.5 ratio (about 50:about 10:about 38.5:about 1.5) or an about 47:10:42:1 ratio (about 47:about 10:about 42:about 1). The structural lipid can be, for example, DSPC (e.g., DSPC (Avanti 850365)), SOPC, DOPC, or DOPE. The cationic/ionizable lipid can be, for example, Dlin-MC3-DMA (e.g., Dlin-MC3-DMA (Biofine International)). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP contains Dlin-MC3-DMA, DSPC, cholesterol, and a PEG lipid in an about 45:9:44:2 ratio (about 45:about 9:about 44:about 2). Another specific example of a suitable LNP contains Dlin-MC3-DMA, DOPE, cholesterol, and PEG lipid or PEG DMG in an about 50:10:39:1 ratio (about 50:about 10:about 39:about 1). Another specific example of a suitable LNP has Dlin-MC3-DMA, DSPC, cholesterol, and PEG2k-DMG at an about 55:10:32.5:2.5 ratio (about 55:about 10:about 32.5:about 2.5). Another specific example of a suitable LNP has Dlin-MC3-DMA, DSPC, cholesterol, and PEG-DMG in an about 50:10:38.5:1.5 ratio (about 50:about 10:about 38.5:about 1.5). Another specific example of a suitable LNP has Dlin-MC3-DMA, DSPC, cholesterol, and PEG-DMG in an about 50:10:38.5:1.5 ratio (about 50:about 10:about 38.5:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Other examples of suitable LNPs can be found, e.g., in WO 2019/067992, WO 2020/082042, US 2020/0270617, WO 2020/082041, US 2020/0268906, WO 2020/082046 (see, e.g., pp. 85-86), and US 2020/0289628, each of which is herein incorporated by reference in its entirety for all purposes.

### (6) Vectors Comprising Nuclease Agents

The nuclease agents disclosed herein (e.g., ZFN, TALEN, or CRISPR/Cas) can be provided in a vector for expression. A vector can comprise additional sequences such as, for example, replication origins, promoters, and genes encoding antibiotic resistance.

Some vectors may be circular. Alternatively, the vector may be linear. The vector can be in the packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

Introduction of nucleic acids can also be accomplished by virus-mediated delivery, such as AAV-mediated delivery or lentivirus-mediated delivery. The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹² to about 10¹⁶ vg/mL. Other exemplary viral titers (e.g., AAV titers) include about 10¹² to about 10¹⁶ vg/kg of body weight.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. Indeed, rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In therapeutic rAAV genomes, a gene expression cassette is placed between ITR sequences. Typically, rAAV genome cassettes comprise of a promoter to drive expression of a therapeutic transgene, followed by polyadenylation sequence. The ITRs flanking a rAAV expression cassette are usually derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

Some non-limiting examples of ITRs that can be used include ITRs comprising, consisting essentially of, or consisting of SEQ ID NO: 158, SEQ ID NO: 159, or SEQ ID NO: 160. Other examples of ITRs comprise one or more mutations compared to SEQ ID NO: 158, SEQ ID NO: 159, or SEQ ID NO: 160 and can be at least 90%, at least 91%, at least 92%, at least 93%, at least 94%, at least 95%, at least 96%, at least 97%, at least 98%, or at least 99% identical to SEQ ID NO: 158, SEQ ID NO: 159, or SEQ ID NO: 160. In some rAAV genomes disclosed herein, the nucleic acid encoding the nuclease agent (or component thereof) is flanked on both sides by the same ITR (i.e., the ITR on the 5′ end, and the reverse complement of the ITR on the 3′ end, such as SEQ ID NO: 158 on the 5′ end and SEQ ID NO: 168 on the 3′ end, or SEQ ID NO: 159 on the 5′ end and SEQ ID NO: 613 on the 3′ end, or SEQ ID NO: 160 on the 5′ end and SEQ ID NO: 614 on the 3′ end). In one example, the ITR on each end can comprise, consist essentially of, or consist of SEQ ID NO: 158 (i.e., SEQ ID NO: 158 on the 5′ end, and the reverse complement on the 3′ end). In another example, the ITR on each end can comprise, consist essentially of, or consist of SEQ ID NO: 159 (i.e., SEQ ID NO: 159 on the 5′ end, and the reverse complement on the 3′ end). In one example, the ITR on at least one end comprises, consists essentially of, or consists of SEQ ID NO: 160. In one example, the ITR on the 5′ end comprises, consists essentially of, or consists of SEQ ID NO: 160. In one example, the ITR on the 3′ end comprises, consists essentially of, or consists of SEQ ID NO: 160. In one example, the ITR on each end can comprise, consist essentially of, or consist of SEQ ID NO: 160 (i.e., SEQ ID NO: 160 on the 5′ end, and the reverse complement on the 3′ end). In one example, the ITR on each end can comprise, consist essentially of, or consist of SEQ ID NO: 160. In other rAAV genomes disclosed herein, the nucleic acid encoding the nuclease agent (or component thereof) is flanked by different ITRs on each end. In one example, the ITR on one end comprises, consists essentially of, or consists of SEQ ID NO: 158, and the ITR on the other end comprises, consists essentially of, or consists of SEQ ID NO: 159. In another example, the ITR on one end comprises, consists essentially of, or consists of SEQ ID NO: 158, and the ITR on the other end comprises, consists essentially of, or consists of SEQ ID NO: 160. In one example, the ITR on one end comprises, consists essentially of, or consists of SEQ ID NO: 159, and the ITR on the other end comprises, consists essentially of, or consists of SEQ ID NO: 160.

The specific serotype of a recombinant AAV vector influences its in vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo. Several serotypes of rAAVs, including rAAV8, are capable of transducing the liver when delivered systemically in mice, NHPs and humans. See, e.g., Li et al. (2020) Nat. Rev. Genet. 21:255-272, herein incorporated by reference in its entirety for all purposes.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

When specific rAAVs comprising specific sequences (e.g., specific bidirectional construct sequences or specific unidirectional construct sequences) are disclosed herein, they are meant to encompass the sequence disclosed or the reverse complement of the sequence. For example, if a bidirectional or unidirectional construct disclosed herein consists of the hypothetical sequence 5′-CTGGACCGA-3′, it is also meant to encompass the reverse complement of that sequence (5′-TCGGTCCAG-3′). Likewise, when rAAVs comprising bidirectional or unidirectional construct elements in a specific 5′ to 3′ order are disclosed herein, they are also meant to encompass the reverse complement of the order of those elements. For example, if an rAAV is disclosed herein that comprises a bidirectional construct that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. A “AAV vector” as used herein refers to an AAV vector comprising a heterologous sequence not of AAV origin (i.e., a nucleic acid sequence heterologous to AAV), typically comprising a sequence encoding an exogenous polypeptide of interest (e.g., multidomain therapeutic protein). The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for liver tissue include AAV3B, AAV5, AAV6, AAV7, AAV8, AAV9, AAVrh.74, and AAVhu.37, and particularly AAV8. In a specific example, the AAV vector comprising the nucleic acid construct can be recombinant AAV8 (rAAV8). A rAAV8 vector as described herein is one in which the capsid is from AAV8. For example, an AAV vector using ITRs from AAV2 and a capsid of AAV8 is considered herein to be a rAAV8 vector.

Tropism can be further refined through pseudotyping, which is the mixing of a capsid and a genome from different viral serotypes. For example, AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

In certain AAVs, the cargo can include nucleic acids encoding one or more guide RNAs (e.g., DNA encoding a guide RNA, or DNA encoding two or more guide RNAs). In certain AAVs, the cargo can include a nucleic acid (e.g., DNA) encoding a Cas nuclease, such as Cas9, and DNA encoding one or more guide RNAs (e.g., DNA encoding a guide RNA, or DNA encoding two or more guide RNAs). In certain AAVs, the cargo can include a nucleic acid construct encoding a multidomain therapeutic protein. In certain AAVs, the cargo can include a nucleic acid (e.g., DNA) encoding a Cas nuclease, such as Cas9, a DNA encoding a guide RNA (or multiple guide RNAs), and a nucleic acid construct encoding a multidomain therapeutic protein.

For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., rAAV8-mediated delivery). For example, a Cas9 mRNA and a gRNA can be delivered via LNP-mediated delivery, or DNA encoding Cas9 and DNA encoding a gRNA can be delivered via AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

### C. Cells or Animals or Genomes

Cells or animals (i.e., subjects) comprising any of the above compositions (e.g., multidomain therapeutic protein, nucleic acid construct encoding a multidomain therapeutic protein, nuclease agents, vectors, lipid nanoparticles, or any combination thereof) are also provided herein. Such cells or animals (or genomes) can be produced by the methods disclosed herein. For example, the cells or animals can comprise any of the multidomain therapeutic proteins described herein, any of the nucleic acid constructs encoding a multidomain therapeutic protein described herein, any of the nuclease agents disclosed herein, or both. Such cells or animals (or genomes) can be neonatal cells or animals (or genomes). Alternatively, such cells or animals (or genomes) can be non-neonatal cells or animals (or genomes).

A neonatal subject (e.g., animal) can be a human subject up to or under the age of 1 year (52 weeks), preferably up to or under the age of 24 weeks, more preferably up to or under the age of 12 weeks, more preferably up to or under the age of 8 weeks, and even more preferably up to or under the age of 4 weeks. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals.

Neonatal cells can be cells of any neonatal subject. For example, they can be of a human subject up to or under the age of 1 year (52 weeks), preferably up to or under the age of 24 weeks, more preferably up to or under the age of 12 weeks, more preferably up to or under the age of 8 weeks, and even more preferably up to or under the age of 4 weeks. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals.

In some such cells or animals or genomes, a nucleic acid construct encoding a multidomain therapeutic protein can be genomically integrated at a target genomic locus, such as a safe harbor locus (e.g., an ALB locus or a human ALB locus, such as intron 1 of an ALB locus or a human ALB locus). In some such cells, animals, or genomes, the multidomain therapeutic protein encoded by the nucleic acid construct is expressed in the cell, animal, or genome. For example, if the nucleic acid construct encoding a multidomain therapeutic protein is integrated into an ALB locus (e.g., intron 1 of a human ALB locus), the multidomain therapeutic protein can be expressed from the ALB locus. The coding sequence for the multidomain therapeutic protein can be operably linked to an endogenous promoter at the target genomic locus upon integration into the target genomic locus, or it can be operably linked to an exogenous promoter present in the nucleic acid construct. If the nucleic acid construct is a bidirectional nucleic acid construct disclosed herein, the genome, cell, or animal can express the first multidomain therapeutic protein or can express the second multidomain therapeutic protein. In some genomes, cells, or animals, the target genomic locus is an ALB locus. For example, the nucleic acid construct can be genomically integrated in intron 1 of the endogenous ALB locus. Endogenous ALB exon 1 can then splice into the coding sequence for the multidomain therapeutic protein in the nucleic acid construct. In some cells, the percentage of unintended transcripts from the target genomic locus containing comprising the integrated nucleic acid construct or coding sequence for the multidomain therapeutic protein is less than about 10%, less than about 9%, less than about 8%, less than about 7%, less than about 6%, less than about 5%, less than about 4%, less than about 3%, less than about 2%, or less than about 1%. In some cells, the percentage of unintended transcripts from the target genomic locus containing comprising the inserted nucleic acid construct or coding sequence for the multidomain therapeutic protein is less than about 5%. In some cells, the percentage of unintended transcripts from the target genomic locus containing comprising the inserted nucleic acid construct or coding sequence for the multidomain therapeutic protein is less than about 4%. In some cells, the percentage of unintended transcripts from the target genomic locus containing comprising the inserted nucleic acid construct or coding sequence for the multidomain therapeutic protein is less than about 3%. In some cells, the percentage of unintended transcripts from the target genomic locus containing comprising the inserted nucleic acid construct or coding sequence for the multidomain therapeutic protein is less than about 2%. In some cells, the percentage of unintended transcripts from the target genomic locus containing comprising the inserted nucleic acid construct or coding sequence for the multidomain therapeutic protein is less than about 1%. The percentage of unintended transcripts means the percentage of all transcripts from the target genomic locus with the inserted nucleic acid construct or coding sequence for the multidomain therapeutic protein that are unintended transcripts and not the intended transcript from the nucleic acid construct being inserted (e.g., transcripts formed by splicing from cryptic splice donors or into cryptic splice acceptors).

The target genomic locus at which the nucleic acid construct is stably integrated can be heterozygous for the nucleic acid construct encoding a multidomain therapeutic protein or homozygous for the nucleic acid construct encoding a multidomain therapeutic protein. A diploid organism has two alleles at each genetic locus. Each pair of alleles represents the genotype of a specific genetic locus. Genotypes are described as homozygous if there are two identical alleles at a particular locus and as heterozygous if the two alleles differ.

The cells, animals, or genomes can be from any suitable species, such as eukaryotic cells or eukaryotes, or mammalian cells or mammals (e.g., non-human mammalian cells or non-human mammals, or human cells or humans). A mammal can be, for example, a non-human mammal, a human, a rodent, a rat, a mouse, or a hamster. Other non-human mammals include, for example, non-human primates, e.g., monkeys and apes. The term “non-human” excludes humans. Examples include, but are not limited to, human cells/humans, rodent cells/rodents, mouse cells/mice, rat cells/rats, and non-human primate cells/non-human primates. In a specific example, the cell is a human cell or the animal is a human. Likewise, cells can be any suitable type of cell. In a specific example, the cell is a liver cell such as a hepatocyte (e.g., a human liver cell or human hepatocyte).

The cells can be isolated cells (e.g., in vitro), ex vivo cells, or can be in vivo within an animal (i.e., in a subject). The cells can be mitotically competent cells or mitotically-inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell. For example, the cells can be liver cells, such as hepatocytes (e.g., mouse, non-human primate, or human hepatocytes).

The cells provided herein can be normal, healthy cells, or can be diseased or mutant-bearing cells. For example, the cells can have a GAA deficiency or can be from a subject with a GAA deficiency. For example, the cells can have a GAA deficiency, can carry a mutation that results in a GAA deficiency, or can be from a subject with a GAA deficiency carrying a mutation that results in a GAA deficiency, or Pompe disease. In some embodiments, the cells are of a neonatal subject.

The cells provided herein can be dividing cells (e.g., actively dividing cells). Alternatively, the cells provided herein can be non-dividing cells.

### III. Therapeutic Methods and Methods for Introducing, Integrating, or Expressing a Nucleic Acid Encoding a Multidomain Therapeutic Protein in Cells or Subjects

The multidomain therapeutic protein nucleic acid constructs and compositions disclosed herein can be used in methods of introducing a nucleic acid construct encoding a multidomain therapeutic protein into a cell or a population of cells or a subject (e.g., in a cell or population of cells in a subject), methods of inserting or integrating a nucleic acid encoding a multidomain therapeutic protein into a target genomic locus in a cell or a population of cells or a subject (e.g., in a cell or population of cells in a subject), methods of expressing a multidomain therapeutic protein in a cell or a population of cells or a subject (e.g., in a cell or population of cells in a subject), methods of reducing glycogen accumulation in a cell or a population of cells or a tissue in a subject (e.g., in a cell or population of cells in a subject), methods of treating Pompe disease or GAA deficiency in a subject, and methods or preventing or reducing the onset of a sign or symptom of Pompe disease or GAA deficiency in a subject. In some methods, the percentage of unintended transcripts from the target genomic locus containing comprising the inserted nucleic acid construct or coding sequence for the multidomain therapeutic protein is less than about 10%, less than about 9%, less than about 8%, less than about 7%, less than about 6%, less than about 5%, less than about 4%, less than about 3%, less than about 2%, or less than about 1%. In some methods, the percentage of unintended transcripts from the target genomic locus containing comprising the inserted nucleic acid construct or coding sequence for the multidomain therapeutic protein is less than about 5%. In some methods, the percentage of unintended transcripts from the target genomic locus containing comprising the inserted nucleic acid construct or coding sequence for the multidomain therapeutic protein is less than about 4%. In some methods, the percentage of unintended transcripts from the target genomic locus containing comprising the inserted nucleic acid construct or coding sequence for the multidomain therapeutic protein is less than about 3%. In some methods, the percentage of unintended transcripts from the target genomic locus containing comprising the inserted nucleic acid construct or coding sequence for the multidomain therapeutic protein is less than about 2%. In some methods, the percentage of unintended transcripts from the target genomic locus containing comprising the inserted nucleic acid construct or coding sequence for the multidomain therapeutic protein is less than about 1%. The percentage of unintended transcripts means the percentage of all transcripts from the target genomic locus with the inserted nucleic acid construct or coding sequence for the multidomain therapeutic protein that are unintended transcripts and not the intended transcript from the nucleic acid construct being inserted (e.g., transcripts formed by splicing from cryptic splice donors or into cryptic splice acceptors).

The multidomain therapeutic protein compositions disclosed herein (e.g., multidomain therapeutic protein nucleic acid constructs, or multidomain therapeutic protein nucleic acid constructs in combination with the nuclease agents (e.g., CRISPR/Cas systems)) are useful for the treatment of GAA deficiency or Pompe disease and/or ameliorating at least one symptom associated with GAA deficiency or Pompe disease (e.g., as compared to a control, untreated subject). The multidomain therapeutic protein compositions disclosed herein (e.g., multidomain therapeutic protein nucleic acid constructs, or multidomain therapeutic protein nucleic acid constructs in combination with the nuclease agents (e.g., CRISPR/Cas systems)) are also useful for preventing or reducing the onset of a sign or symptom of GAA deficiency or Pompe disease (e.g., as compared to a control, untreated subject). Likewise, the compositions disclosed herein can be used for the preparation of a pharmaceutical composition or medicament for treating a subject having GAA deficiency or Pompe disease.

With respect to GAA deficiency or Pompe disease, the terms “treat,” “treated,” “treating,” and “treatment,” include the administration of the multidomain therapeutic domain nucleic acid constructs disclosed herein (e.g., together with a nuclease agent disclosed herein) to subjects to prevent or delay the onset of the symptoms, complications, or biochemical indicia of GAA deficiency or Pompe disease, alleviating the symptoms or arresting or inhibiting further development of GAA deficiency or Pompe disease. Treatment may be prophylactic (to prevent or delay the onset of GAA deficiency or Pompe disease, or to prevent the manifestation of clinical or subclinical symptoms thereof) or therapeutic suppression or alleviation of symptoms after the manifestation of GAA deficiency or Pompe disease.

GAA deficiency refers expression and/or activity levels of GAA being lower in the subject (e.g., neonatal subject) than normal GAA expression and/or activity levels, such that the normal functions of GAA are not fully carried out in the subject (e.g., resulting in Pompe disease). Pompe disease is also known as acid maltase deficiency, acid maltase deficiency disease, alpha-1,4-glucosidase deficiency, AMD, deficiency of alpha-glucosidase, GAA deficiency, glycogen storage disease type II, glycogenosis type II, GSD II, GSD2, and Pompe's disease.

Pompe disease is an inherited disorder caused by the buildup of glycogen in the body's cells. The accumulation of glycogen in certain organs and tissues, especially muscles, impairs their ability to function normally. Different types of Pompe disease differ in severity and the age at which they appear. These types are known as infantile-onset Pompe disease (classic infantile-onset, and non-classic infantile-onset) and late-onset Pompe disease. Subjects with late-onset Pompe disease have higher GAA enzyme levels than are found in infantile-onset forms of the disease, but generally less than 40 percent of normal enzyme activity. Classic infantile-onset Pompe disease patients typically have less than 1 percent of GAA enzyme activity, while those with non-classic forms usually have less than 10 percent.

The classic form of infantile-onset Pompe disease begins within a few months of birth. Some phenotypes, such as cardiomyopathies, can be present at birth. Infants with this disorder typically experience muscle weakness (myopathy), poor muscle tone (hypotonia), an enlarged liver (hepatomegaly), and heart defects. Affected infants may also fail to gain weight and grow at the expected rate (failure to thrive) and have breathing problems. If untreated, this form of Pompe disease leads to death from heart failure in the first year of life.

The non-classic form of infantile-onset Pompe disease usually appears by age 1. It is characterized by delayed motor skills (such as rolling over and sitting) and progressive muscle weakness. The heart may be abnormally large (cardiomegaly), but affected individuals usually do not experience heart failure. The muscle weakness in this disorder leads to serious breathing problems, and most children with non-classic infantile-onset Pompe disease live only into early childhood.

The late-onset type of Pompe disease may not become apparent until later in childhood, adolescence, or adulthood. Late-onset Pompe disease is usually milder than the infantile-onset forms of this disorder and is less likely to involve the heart. Most individuals with late-onset Pompe disease experience progressive muscle weakness, especially in the legs and the trunk, including the muscles that control breathing. As the disorder progresses, breathing problems can lead to respiratory failure.

Mutations in the GAA gene can cause Pompe disease. The GAA gene encodes an enzyme called alpha-glucosidase. This enzyme is active in lysosomes. The enzyme normally breaks down glycogen into glucose. Mutations in the GAA gene prevent acid alpha-glucosidase from breaking down glycogen effectively, which allows this sugar to build up to toxic levels in lysosomes. This buildup damages organs and tissues throughout the body, particularly the muscles, leading to the progressive signs and symptoms of Pompe disease.

Since this is a genetic condition, the people who get this disease inherit it from a parent. It is common, however, that neither parent shows any symptoms. The disease is rare. In the United States, only 1 person in 40,000 is affected by Pompe disease. It can affect both males and females of all ethnic groups.

Symptoms of Pompe disease can be different, depending on when the disease makes itself present. For classic type, infant symptoms can include the following: weak muscles, poor muscle tone, enlarged liver, failure to gain weight and grow at the expected rate (failure to thrive), trouble breathing, feeding problems, infections in the respiratory system, and problems with hearing. For non-classic type, infant symptoms can include the following: motor skills delayed (such as rolling over and sitting), muscles get steadily weaker, abnormally large heart, and breathing problems. For late-onset type, symptoms can include the following: the legs and the trunk get steadily weaker, breathing problems, enlarged heart, increasing difficulty in walking, muscle pain over a large area, loss of the ability to exercise, falling often, frequent lung infections, shortness of breath when the person pushes himself or herself, headaches in the morning, becoming tired during the day, losing weight, cannot swallow as easily as before, irregular heartbeat, increased difficulty hearing, and higher levels of creatine kinase.

Pathology in Pompe disease can begin long before subjects present with symptoms. Pompe disease can be diagnosed by taking a blood sample, and enzymes in the blood are studied and counted. Confirmation can be made via DNA testing. For example, GAA enzyme activity can be measured by flow-injection tandem mass spectrometry, and full sequencing of the GAA gene is performed in newborns with low GAA enzyme activity. See, e.g., Ficicioglu et al. (2020) Int. J. Neonatal Screen. 6(4):89, Tang et al. (2020) Int. J. Neonatal Screen. 6(1):9, and Klug et al. (2020) Int. J. Neonatal Screen 6(1):11, each of which is herein incorporated by reference in its entirety for all purposes. GAA activity can be assessed by any known method. For example, to assess GAA activity (or deficiencies of activity), blood-based assays can measure GAA activity in dried blood spots or fresh blood. GAA activity can also be measured in fibroblasts from a skin biopsy or muscle biopsy. Other secondary measures can be measuring urine glucose tetrasaccharides by mass spectrometry. These can be combined with genetic analyses to diagnose in infantile and late onset Pompe disease. Asymptomatic subjects can be considered to have Pompe disease if diagnosed by genetic screening. For example, a subject described herein is considered to have Pompe disease, even if they are asymptomatic, if they have reduced GAA activity and a pathogenic GAA variant or mutation. Pathogenic GAA mutations and variants associated with Pompe disease are known. See, e.g., Ficicioglu et al. (2020) Int. J. Neonatal Screen. 6(4):89, Tang et al. (2020) Int. J. Neonatal Screen. 6(1):9, and Klug et al. (2020) Int. J. Neonatal Screen 6(1):11, each of which is herein incorporated by reference in its entirety for all purposes.

As is the case for several other lysosomal diseases, Pompe disease is currently treated by enzyme replacement therapy (ERT). Recombinant human GAA is delivered by intravenous infusion into patients every other week. While ERT has been successful in treating the cardiac manifestations of Pompe disease, skeletal muscle and the central nervous system (CNS) remain minimally treated by ERT.

The cells or populations of cells can be neonatal cells or populations of neonatal cells, and the subject can be neonatal subjects in some methods of introducing a nucleic acid construct encoding a multidomain therapeutic protein into a cell or a population of cells or a subject (e.g., in a cell or population of cells in a subject), methods of inserting or integrating a nucleic acid encoding the multidomain therapeutic protein into a target genomic locus in a cell or a population of cells or a subject (e.g., in a cell or population of cells in a subject), methods of expressing a multidomain therapeutic protein in a cell or a population of cells or a subject (e.g., in a cell or population of cells in a subject), methods of reducing glycogen accumulation in a cell or a population of cells or a tissue in a subject (e.g., in a cell or population of cells in a subject), and methods of treating Pompe disease or GAA deficiency in a subject. A neonatal subject can be a human subject up to or under the age of 1 year (52 weeks), preferably up to or under the age of 24 weeks, more preferably up to or under the age of 12 weeks, more preferably up to or under the age of 8 weeks, and even more preferably up to or under the age of 4 weeks. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals. As used herein, a “neonatal cell” is a cell of a neonatal subject, and a population of neonatal cells is a population of cells of a neonatal subject. In other methods, the cells or populations of cells are not neonatal cells and are not populations of neonatal cells, and the subjects are not neonatal subjects.

In one example, provided herein are methods of introducing a nucleic acid encoding a multidomain therapeutic protein into a cell or a population of cells or a subject in need thereof (e.g., in a cell or a population of cells in the subject). The cells or populations of cells can be neonatal cells or populations of neonatal cells, and the subject can be neonatal subjects in some methods. In other methods, the cells or populations of cells are not neonatal cells and are not populations of neonatal cells, and the subjects are not neonatal subjects. Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the cell. The multidomain therapeutic protein nucleic acid construct can be administered together with a nuclease agent described herein, or can be administered alone. For example, the multidomain therapeutic protein nucleic acid construct can be one that expresses the multidomain therapeutic protein without being integrated into target genomic locus (e.g., an episomal vector or an expression vector in which the coding sequence for the multidomain therapeutic protein is operably linked to a promoter). In some methods, the multidomain therapeutic protein nucleic acid construct can be administered together with a nuclease agent described herein (e.g., simultaneously or sequentially in any order). The nuclease agent can cleave a nuclease target sequence within a target genomic locus (e.g., target gene), the nucleic acid encoding the multidomain therapeutic protein can be inserted into the target genomic locus to create a modified target genomic locus, and the multidomain therapeutic protein can be expressed from the modified target genomic locus. The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence, the nucleic acid encoding the multidomain therapeutic protein can be inserted into the ALB gene to create a modified ALB gene, and multidomain therapeutic protein can be expressed from the modified ALB gene.

In another example, provided herein are methods of expressing a multidomain therapeutic protein in a cell or a population of cells or a subject in need thereof (e.g., in a cell or a population of cells in the subject). The cells or populations of cells can be neonatal cells or populations of neonatal cells, and the subject can be neonatal subjects in some methods. In other methods, the cells or populations of cells are not neonatal cells and are not populations of neonatal cells, and the subjects are not neonatal subjects. Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the cell. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of the multidomain therapeutic protein without integration into a target genomic locus). In some methods, the multidomain therapeutic protein nucleic acid construct can be administered together with a nuclease agent described herein (e.g., simultaneously or sequentially in any order). The nuclease agent can cleave a nuclease target sequence within a target genomic locus (e.g., target gene), the nucleic acid encoding the multidomain therapeutic protein can be inserted into the target genomic locus to create a modified target genomic locus, and multidomain therapeutic protein can be expressed from the modified target genomic locus. The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence, the nucleic acid encoding the multidomain therapeutic protein can be inserted into the ALB gene to create a modified ALB gene, and multidomain therapeutic protein can be expressed from the modified ALB gene.

In another example, provided herein are methods of inserting or integrating a nucleic acid encoding the multidomain therapeutic protein into a target genomic locus in a cell or a population of cells or a subject in need thereof (e.g., in a cell or a population of cells in the subject). The cells or populations of cells can be neonatal cells or populations of neonatal cells, and the subject can be neonatal subjects in some methods. In other methods, the cells or populations of cells are not neonatal cells and are not populations of neonatal cells, and the subjects are not neonatal subjects. Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the cell. In some methods, the multidomain therapeutic protein nucleic acid construct or composition comprising the multidomain therapeutic protein nucleic acid construct can be administered together with a nuclease agent described herein (e.g., simultaneously or sequentially in any order). The nuclease agent can cleave a nuclease target sequence within a target genomic locus (e.g., target gene), the nucleic acid encoding the multidomain therapeutic protein can be inserted into the target genomic locus to create a modified target genomic locus, and the multidomain therapeutic protein can be expressed from the modified target genomic locus. The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence, the nucleic acid encoding the multidomain therapeutic protein can be inserted into the ALB gene to create a modified ALB gene, and multidomain therapeutic protein can be expressed from the modified ALB gene.

In any of the above methods, the cells can be from any suitable species, such as eukaryotic cells or mammalian cells (e.g., non-human mammalian cells or human cells). A mammal can be, for example, a non-human mammal, a human, a rodent, a rat, a mouse, or a hamster. Other non-human mammals include, for example, non-human primates, e.g., monkeys and apes. The term “non-human” excludes humans. Specific examples include, but are not limited to, human cells, rodent cells, mouse cells, rat cells, and non-human primate cells. In a specific example, the cell is a human cell. Likewise, cells can be any suitable type of cell. In a specific example, the cell is a liver cell such as a hepatocyte (e.g., a human liver cell or human hepatocyte). The cells can be neonatal cells, or they can be non-neonatal cells.

The cells can be isolated cells (e.g., in vitro), ex vivo cells, or can be in vivo within an animal (i.e., in a subject). In a specific example, the cell is in vivo (e.g., in a subject having a GAA deficiency or Pompe disease). The cells can be mitotically competent cells or mitotically-inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell. For example, the cells can be liver cells, such as hepatocytes (e.g., mouse, non-human primate, or human hepatocytes).

The cells provided herein can be normal, healthy cells, or can be diseased or mutant-bearing cells. For example, the cells can have a GAA deficiency or can be from a subject with GAA deficiency or Pompe disease.

Also provided are methods of treating a lysosomal alpha-glucosidase (GAA) deficiency in a subject in need thereof (e.g., a subject with Pompe disease). The Pompe disease can be any type of Pompe disease (e.g., infantile-onset Pompe disease (classic infantile-onset or non-classic infantile-onset) or late-onset Pompe disease). For example, the subject can have infantile-onset Pompe disease (e.g., classical infantile-onset Pompe disease). Pompe disease is described in more detail elsewhere herein. In some methods, the expressed multidomain therapeutic protein is delivered to and internalized by skeletal muscle, liver, or heart tissue in the subject. In some methods, the expressed multidomain therapeutic protein is delivered to and internalized by skeletal muscle or heart tissue in the subject. In some methods, the expressed multidomain therapeutic protein is delivered to and internalized by skeletal muscle tissue in the subject. In some methods, the expressed multidomain therapeutic protein is delivered to and internalized by liver tissue in the subject. In some methods, the expressed multidomain therapeutic protein is delivered to and internalized by heart tissue in the subject. In some methods, the expressed multidomain therapeutic protein is delivered to and internalized by skeletal muscle, liver, and heart tissue in the subject. In some methods, the expressed multidomain therapeutic protein is delivered to and internalized by skeletal muscle and heart tissue in the subject. In some methods, the expressed multidomain therapeutic protein is delivered to and internalized by skeletal muscle, liver, heart, or central nervous system tissue in the subject. In some methods, the expressed multidomain therapeutic protein is delivered to and internalized by skeletal muscle, heart, or central nervous system tissue in the subject. In some methods, the expressed multidomain therapeutic protein is delivered to and internalized by skeletal muscle tissue in the subject. In some methods, the expressed multidomain therapeutic protein is delivered to and internalized by liver tissue in the subject. In some methods, the expressed multidomain therapeutic protein is delivered to and internalized by heart tissue in the subject. In some methods, the expressed multidomain therapeutic protein is delivered to and internalized by central nervous system tissue in the subject. In some methods, the expressed multidomain therapeutic protein is delivered to and internalized by skeletal muscle, liver, heart, and central nervous system tissue in the subject. In some methods, the expressed multidomain therapeutic protein is delivered to and internalized by skeletal muscle, heart, and central nervous system tissue in the subject. In some methods, the method reduces glycogen accumulation in skeletal muscle, liver, or heart tissue in the subject. In some methods, the method reduces glycogen accumulation in skeletal muscle or heart tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. For example, glycogen accumulation can be reduced in skeletal muscle, liver, and heart tissue in the subject. For example, glycogen accumulation can be reduced in skeletal muscle and heart tissue in the subject. In some methods, the method reduces glycogen accumulation in skeletal muscle, liver, heart, or central nervous system tissue in the subject. In some methods, the method reduces glycogen accumulation in skeletal muscle, heart, or central nervous system tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. In some methods, the method reduces glycogen accumulation in central nervous system tissue in the subject. For example, glycogen accumulation can be reduced in skeletal muscle, liver, heart, and central nervous system tissue in the subject. For example, glycogen accumulation can be reduced in skeletal muscle, heart, and central nervous system tissue in the subject. In some cases, glycogen levels are reduced to wild type levels. In some cases, glycogen levels in skeletal muscle, heart, and/or central nervous system tissue are reduced to levels comparable to wild type levels at the same age. In some methods, the method improves muscle strength in the subject (e.g., restores muscle strength to wild type levels). In some methods, the method prevents loss of muscle strength in the subject compared to a control. In some methods, the method results in the subject having muscle strength comparable to wild type levels at the same age. Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject such that a therapeutically effective level of multidomain therapeutic protein or GAA expression or a therapeutically effective level of circulating multidomain therapeutic protein or GAA is achieved in the subject. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The nuclease agent can cleave a nuclease target sequence within a target genomic locus (e.g., target gene), the nucleic acid encoding the multidomain therapeutic protein can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid encoding the multidomain therapeutic protein can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

Also provided are methods of reducing glycogen accumulation in a cell or a population of cells or a tissue in a subject in need thereof (e.g., a subject with Pompe disease). Similarly, provided are methods of reducing glycogen accumulation in a cell or a population of cells. The Pompe disease can be any type of Pompe disease (e.g., infantile-onset Pompe disease (classic infantile-onset or non-classic infantile-onset) or late-onset Pompe disease). For example, the subject can have infantile-onset Pompe disease (e.g., classical infantile-onset Pompe disease). Pompe disease is described in more detail elsewhere herein. In some methods, the method reduces glycogen accumulation in skeletal muscle, liver, or heart tissue in the subject. In some methods, the method reduces glycogen accumulation in skeletal muscle or heart tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. For example, glycogen accumulation can be reduced in skeletal muscle, liver, and heart tissue in the subject. For example, glycogen accumulation can be reduced in skeletal muscle and heart tissue in the subject. In some methods, the method reduces glycogen accumulation in skeletal muscle, liver, heart, or central nervous system tissue in the subject. In some methods, the method reduces glycogen accumulation in skeletal muscle, heart, or central nervous system tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. In some methods, the method reduces glycogen accumulation in central nervous system tissue in the subject. For example, glycogen accumulation can be reduced in skeletal muscle, liver, heart, and central nervous system tissue in the subject. For example, glycogen accumulation can be reduced in skeletal muscle, heart, and central nervous system tissue in the subject. In some cases, glycogen levels are reduced to wild type levels. In some cases, glycogen levels in skeletal muscle, heart, and/or central nervous system tissue are reduced to levels comparable to wild type levels at the same age. In some methods, the method improves muscle strength in the subject (e.g., restores muscle strength to wild type levels). In some methods, the method prevents loss of muscle strength in the subject compared to a control. In some methods, the method results in the subject having muscle strength comparable to wild type levels at the same age. Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject such that a therapeutically effective level of multidomain therapeutic protein or GAA expression or a therapeutically effective level of circulating multidomain therapeutic protein or GAA is achieved in the subject. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The nuclease agent can cleave a nuclease target sequence within a target genomic locus (e.g., target gene), the nucleic acid encoding the multidomain therapeutic protein can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid encoding the multidomain therapeutic protein can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

Also provided are methods of treating Pompe disease in a subject. The Pompe disease can be any type of Pompe disease (e.g., infantile-onset Pompe disease (classic infantile-onset or non-classic infantile-onset) or late-onset Pompe disease). For example, the subject can have infantile-onset Pompe disease (e.g., classical infantile-onset Pompe disease). Pompe disease is described in more detail elsewhere herein. In some methods, the method reduces glycogen accumulation in skeletal muscle, liver, or heart tissue in the subject. In some methods, the method reduces glycogen accumulation in skeletal muscle or heart tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. For example, glycogen accumulation can be reduced in skeletal muscle, liver, and heart tissue in the subject. For example, glycogen accumulation can be reduced in skeletal muscle and heart tissue in the subject. In some methods, the method reduces glycogen accumulation in skeletal muscle, liver, heart, or central nervous system tissue in the subject. In some methods, the method reduces glycogen accumulation in skeletal muscle, heart, or central nervous system tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. In some methods, the method reduces glycogen accumulation in central nervous system tissue in the subject. For example, glycogen accumulation can be reduced in skeletal muscle, liver, heart, and central nervous system tissue in the subject. For example, glycogen accumulation can be reduced in skeletal muscle, heart, and central nervous system tissue in the subject. In some cases, glycogen levels are reduced to wild type levels. In some cases, glycogen levels in skeletal muscle, heart, and/or central nervous system tissue are reduced to levels comparable to wild type levels at the same age. In some methods, the method improves muscle strength in the subject (e.g., restores muscle strength to wild type levels). In some methods, the method prevents loss of muscle strength in the subject compared to a control. In some methods, the method results in the subject having muscle strength comparable to wild type levels at the same age. Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject such that a therapeutically effective level of multidomain therapeutic protein or GAA expression or a therapeutically effective level of circulating multidomain therapeutic protein or GAA is achieved in the subject. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The nuclease agent can cleave a nuclease target sequence within a target genomic locus (e.g., target gene), the nucleic acid encoding the multidomain therapeutic protein can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid encoding the multidomain therapeutic protein can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

Treatment refers to any administration or application of a therapeutic for disease or disorder in a subject, and includes inhibiting the disease, arresting its development, relieving one or more symptoms of the disease, curing the disease, or preventing reoccurrence of one or more symptoms of the disease. For example, treatment of Pompe disease may comprise alleviating symptoms of Pompe disease. Pompe disease is described in detail above and refers to a disorder caused by a missing or defective GAA gene or GAA polypeptide. The defective GAA gene or GAA polypeptide can result in reduced GAA expression and/or activity of GAA.

Also provided are methods of preventing or reducing the onset of a sign or symptom of Pompe disease in a subject (e.g., as compared to an untreated, control subject). By preventing is meant the sign or symptom of the Pompe disease never becomes present. Such signs and symptoms are well-known and are described in more detail elsewhere herein. The Pompe disease can be any type of Pompe disease (e.g., infantile-onset Pompe disease (classic infantile-onset or non-classic infantile-onset) or late-onset Pompe disease). For example, Pompe disease can be infantile-onset Pompe disease (e.g., classical infantile-onset Pompe disease). Pompe disease is described in more detail elsewhere herein. In some methods, the method prevents or reduces glycogen accumulation in skeletal muscle, liver, or heart tissue in the subject. In some methods, the method prevents or reduces glycogen accumulation in skeletal muscle or heart tissue in the subject. In some methods, the method prevents or reduces glycogen accumulation in skeletal muscle tissue in the subject. In some methods, the method prevents or reduces glycogen accumulation in liver tissue in the subject. In some methods, the method prevents or reduces glycogen accumulation in heart tissue in the subject. For example, glycogen accumulation can be prevented or reduced in skeletal muscle, liver, and heart tissue in the subject. For example, glycogen accumulation can be prevented or reduced in skeletal muscle and heart tissue in the subject. In some methods, the method prevents or reduces glycogen accumulation in skeletal muscle, liver, heart, or central nervous system tissue in the subject. In some methods, the method prevents or reduces glycogen accumulation in skeletal muscle, heart, or central nervous system tissue in the subject. In some methods, the method prevents or reduces glycogen accumulation in skeletal muscle tissue in the subject. In some methods, the method prevents or reduces glycogen accumulation in liver tissue in the subject. In some methods, the method prevents or reduces glycogen accumulation in heart tissue in the subject. In some methods, the method prevents or reduces glycogen accumulation in central nervous system tissue in the subject. For example, glycogen accumulation can be prevented or reduced in skeletal muscle, liver, heart, and central nervous system tissue in the subject. For example, the onset of glycogen accumulation can be prevented or reduced in skeletal muscle, heart, and central nervous system tissue in the subject. Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject such that a therapeutically effective level of multidomain therapeutic protein or GAA expression or a therapeutically effective level of circulating multidomain therapeutic protein or GAA is achieved in the subject. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The nuclease agent can cleave a nuclease target sequence within a target genomic locus (e.g., target gene), the nucleic acid encoding the multidomain therapeutic protein can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid encoding the multidomain therapeutic protein can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

In some methods, a therapeutically effective amount of the multidomain therapeutic protein nucleic acid construct or the composition comprising the multidomain therapeutic protein nucleic acid construct or the combination of the multidomain therapeutic protein nucleic acid construct and the nuclease agent (e.g., CRISPR/Cas system) is administered to the subject. A therapeutically effective amount is an amount that produces the desired effect for which it is administered. The exact amount will depend on the purpose of the treatment, and will be ascertainable by one skilled in the art using known techniques. See, e.g., Lloyd (1999) The Art, Science and Technology of Pharmaceutical Compounding. In a specific example, serum levels of at least about 2 μg/mL or at least about 5 μg/mL of the multidomain therapeutic protein are considered therapeutically effective and correspond to complete correction of glycogen storage in muscles.

Therapeutic or pharmaceutical compositions comprising the compositions disclosed herein can be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. See also Powell et al. “Compendium of excipients for parenteral formulations” PDA (1998) J. Pharm. Sci. Technol. 52:238-311. In certain embodiments, the pharmaceutical compositions are non-pyrogenic.

The compositions disclosed herein may be administered to relieve or prevent or decrease the severity of one or more of the symptoms of GAA deficiency or Pompe disease. Such symptoms are described in more detail elsewhere herein.

The subject in any of the above methods can be one in need of amelioration or treatment of GAA deficiency or Pompe disease. The subject in any of the above methods can be from any suitable species, such as a eukaryote or a mammal. A mammal can be, for example, a non-human mammal, a human, a rodent, a rat, a mouse, or a hamster. Other non-human mammals include, for example, non-human primates, e.g., monkeys and apes. The term “non-human” excludes humans. Specific examples of suitable species include, but are not limited to, humans, rodents, mice, rats, and non-human primates. In a specific example, the subject is a human. The subject in some methods can be a neonatal subject. In other methods, the subject is not a neonatal subject.

In methods in which a nucleic acid encoding the multidomain therapeutic protein is genomically integrated, any target genomic locus capable of expressing a gene can be used, such as a safe harbor locus (safe harbor gene) or an endogenous GAA locus. Such loci are described in more detail elsewhere herein. In a specific example, the target genomic locus can be an endogenous ALB locus, such as an endogenous human ALB locus. For example, the nucleic acid encoding the multidomain therapeutic protein can be genomically integrated in intron 1 of the endogenous ALB locus. Endogenous ALB exon 1 can then splice into the coding sequence for the multidomain therapeutic protein in the nucleic acid construct.

Targeted insertion of the nucleic acid encoding the multidomain therapeutic protein into a target genomic locus, and particularly an endogenous ALB locus, offers multiple advantages. Such methods result in stable modification to allow for stable, long-term expression of the multidomain therapeutic protein coding sequence. With respect to the ALB locus, such methods are able to utilize the endogenous ALB promoter and regulatory regions to achieve therapeutically effective levels of expression. For example, the multidomain therapeutic protein coding sequence in the nucleic acid construct can comprise a promoterless gene, and the inserted nucleic acid can be operably linked to an endogenous promoter in the target genomic locus (e.g., ALB locus). Use of an endogenous promoter is advantageous because it obviates the need for inclusion of a promoter in the nucleic acid construct, allowing packaging of larger transgenes that may not normally package efficiently (e.g., in AAV). Alternatively, the multidomain therapeutic protein coding sequence in the nucleic acid construct can be operably linked to an exogenous promoter in the nucleic acid construct. Examples of types of promoters that can be used are disclosed elsewhere herein.

Optionally, some or all of the endogenous gene (e.g., endogenous ALB gene) at the target genomic locus can be expressed upon insertion of the nucleic acid encoding the multidomain therapeutic protein from the nucleic acid construct. Alternatively, in some methods, none of the endogenous gene at the target genomic locus is expressed. As one example, the modified target genomic locus (e.g., modified ALB locus) after integration of the nucleic acid encoding the multidomain therapeutic protein can encode a chimeric protein comprising an endogenous secretion signal (e.g., albumin secretion signal) and the multidomain therapeutic protein encoded by the nucleic acid construct. In another example, the first intron of an ALB locus can be targeted. The secretion signal peptide of ALB is encoded by exon 1 of the ALB gene. In such a scenario, a promoterless cassette bearing a splice acceptor and the multidomain therapeutic protein coding sequence will support expression and secretion of the multidomain therapeutic protein. Splicing between endogenous ALB exon 1 and the integrated multidomain therapeutic protein coding sequence creates a chimeric mRNA and protein including the endogenous ALB sequence encoded by exon 1 operably linked to the multidomain therapeutic protein sequence encoded by the integrated nucleic acid.

The nucleic acid encoding the multidomain therapeutic protein can be inserted into the target genomic locus by any means, including homologous recombination (HR) and non-homologous end joining (NHEJ) as described elsewhere herein. In a specific example, the nucleic acid encoding the multidomain therapeutic protein is inserted by NHEJ (e.g., does not comprise a homology arm and is inserted by NHEJ).

In another specific example, the nucleic acid encoding the multidomain therapeutic protein can be inserted via homology-independent targeted integration (e.g., directional homology-independent targeted integration). For example, the multidomain therapeutic protein coding sequence in the nucleic acid construct can be flanked on each side by a target site for a nuclease agent (e.g., the same target site as in the target genomic locus, and the same nuclease agent being used to cleave the target site in the target genomic locus). The nuclease agent can then cleave the target sites flanking the multidomain therapeutic protein coding sequence. In a specific example, the nucleic acid construct is delivered AAV-mediated delivery, and cleavage of the target sites flanking the multidomain therapeutic protein coding sequence can remove the inverted terminal repeats (ITRs) of the AAV. Removal of the ITRs can make it easier to assess successful targeting, because presence of the ITRs can hamper sequencing efforts due to the repeated sequences. In some methods, the target site in the target genomic locus (e.g., a gRNA target sequence including the flanking protospacer adjacent motif) is no longer present if the multidomain therapeutic protein coding sequence is inserted into the target genomic locus in the correct orientation but it is reformed if the multidomain therapeutic protein coding sequence is inserted into the target genomic locus in the opposite orientation. This can help ensure that the multidomain therapeutic protein coding sequence is inserted in the correct orientation for expression.

In any of the above methods, the multidomain therapeutic protein nucleic acid construct can be administered simultaneously with the nuclease agent (e.g., CRISPR/Cas system) or not simultaneously (e.g., sequentially in any combination). For example, in a method comprising administering a composition comprising the multidomain therapeutic protein nucleic acid construct and a nuclease agent, they can be administered separately. For example, the multidomain therapeutic protein nucleic acid construct can be administered prior to the nuclease agent, subsequent to the nuclease agent, or at the same time as the nuclease agent. Any suitable methods of administering nucleic acid constructs and nuclease agents to cells can be used, particularly methods of administering to the liver, and examples of such methods are described in more detail elsewhere herein. In methods of treatment or in methods of targeting a cell in vivo in a subject, the nucleic acid encoding the multidomain therapeutic protein can be inserted in particular types of cells in the subject. The method and vehicle for introducing the multidomain therapeutic protein nucleic acid construct and/or the nuclease agent into the subject can affect which types of cells in the subject are targeted. In some methods, for example, the nucleic acid encoding the multidomain therapeutic protein is inserted into a target genomic locus (e.g., an endogenous ALB locus) in liver cells, such as hepatocytes. Methods and vehicles for introducing such constructs and nuclease agents into the subject (including methods and vehicles that target the liver or hepatocytes, such as lipid nanoparticle-mediated delivery and AAV-mediated delivery (e.g., rAAV8-mediated delivery) and intravenous injection), are disclosed in more detail elsewhere herein.

In methods in which a composition comprising a nucleic acid construct (or vector or LNP) and a nuclease agent is administered (i.e., in methods in which a nucleic acid construct (or vector or LNP) and a nuclease agent are both administered), the nucleic acid construct and the nuclease agent can be administered simultaneously. Alternatively, the nucleic acid construct and the nuclease agent can be administered sequentially in any order. For example, the nucleic acid construct can be administered after the nuclease agent, or the nuclease agent can be administered after the nucleic acid construct. For example, the nuclease agent can be administered about 1 hour to about 48 hours, about 1 hour to about 24 hours, about 1 hour to about 12 hours, about 1 hour to about 6 hours, about 1 hour to about 2 hours, about 2 hours to about 48 hours, about 2 hours to about 24 hours, about 2 hours to about 12 hours, about 2 hours to about 6 hours, about 3 hours to about 48 hours, about 6 hours to about 48 hours, about 12 hours to about 48 hours, or about 24 hours to about 48 hours prior to or subsequent to administration of the nucleic acid construct.

In one example, the nucleic acid construct is administered about 4 hours, about 8 hours, about 12 hours, about 18 hours, about 1 day, about 2 days, about 3 days, about 4 days, about 5 days, about 6 days, or about 1 week prior to administering the nuclease agent. In another example, the nucleic acid construct is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to administering the nuclease agent. In another example, the nucleic acid construct is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to administering the nuclease agent.

In one example, the nucleic acid construct is administered about 4 hours, about 8 hours, about 12 hours, about 18 hours, about 1 day, about 2 days, about 3 days, about 4 days, about 5 days, about 6 days, or about 1 week after administering the nuclease agent. In another example, the nucleic acid construct is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week after administering the nuclease agent. In another example, the nucleic acid construct is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days after administering the nuclease agent.

In any of the above methods, the multidomain therapeutic protein nucleic acid construct and the nuclease agent (e.g., CRISPR/Cas system) can be administered using any suitable delivery system and known method. The nuclease agent components and multidomain therapeutic protein nucleic acid construct (e.g., the guide RNA, Cas protein, and multidomain therapeutic protein nucleic acid construct) can be delivered individually or together in any combination, using the same or different delivery methods as appropriate.

In methods in which a CRISPR/Cas system is used, a guide RNA can be introduced into or administered to a subject or cell, for example, in the form of an RNA (e.g., in vitro transcribed RNA, such as the modified guide RNAs disclosed herein) or in the form of a DNA encoding the guide RNA. When introduced in the form of a DNA, the DNA encoding a guide RNA can be operably linked to a promoter active in the cell or in a cell in the subject. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be introduced into a subject or cell in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)), such as a modified mRNA as disclosed herein, or DNA). Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or a subject, the Cas protein can be transiently, conditionally, or constitutively expressed in the cell or in a cell in the subject.

In one example, the Cas protein is introduced in the form of an mRNA (e.g., a modified mRNA as disclosed herein), and the guide RNA is introduced in the form of RNA such as a modified gRNA as disclosed herein (e.g., together within the same lipid nanoparticle). Guide RNAs can be modified as disclosed elsewhere herein. Likewise, Cas mRNAs can be modified as disclosed elsewhere herein.

In methods in which a nucleic acid encoding the multidomain therapeutic protein is inserted following cleavage by a gene-editing system (e.g., a Cas protein), the gene-editing system (e.g., Cas protein) can cleave the target genomic locus to create a single-strand break (nick) or double-strand break, and the cleaved or nicked locus can be repaired by insertion of the nucleic acid encoding the multidomain therapeutic protein via non-homologous end joining (NHEJ)-mediated insertion or homology-directed repair. Optionally, repair with the multidomain therapeutic protein nucleic acid construct removes or disrupts the guide RNA target sequence(s) so that alleles that have been targeted cannot be re-targeted by the CRISPR/Cas reagents.

As explained in more detail elsewhere herein, the multidomain therapeutic protein nucleic acid constructs can comprise deoxyribonucleic acid (DNA) or ribonucleic acid (RNA), they can be single-stranded or double-stranded, and they can be in linear or circular form. The multidomain therapeutic protein nucleic acid constructs can be naked nucleic acids or can be delivered by viruses, such as AAV. In a specific example, the multidomain therapeutic protein nucleic acid construct can be delivered via AAV and can be capable of insertion into the target genomic locus (e.g., a safe harbor gene, an ALB gene, or intron 1 of an ALB gene) by non-homologous end joining (e.g., the multidomain therapeutic protein nucleic acid construct can be one that does not comprise a homology arm).

Some nucleic acids encoding the multidomain therapeutic protein are capable of insertion by non-homologous end joining. In some cases, such multidomain therapeutic protein nucleic acid constructs do not comprise a homology arm. For example, such nucleic acids encoding the multidomain therapeutic protein constructs can be inserted into a blunt end double-strand break following cleavage with a Cas protein. In a specific example, the multidomain therapeutic protein nucleic acid construct can be delivered via AAV and can be capable of insertion by non-homologous end joining (e.g., the multidomain therapeutic protein nucleic acid construct can be one that does not comprise a homology arm).

In another example, the nucleic acid encoding the multidomain therapeutic protein can be inserted via homology-independent targeted integration. For example, the multidomain therapeutic protein nucleic acid construct can be flanked on each side by a guide RNA target sequence (e.g., the same target site as in the target genomic locus, and the CRISPR/Cas reagent (Cas protein and guide RNA) being used to cleave the target site in the target genomic locus). The Cas protein can then cleave the target sites flanking the nucleic acid insert. In a specific example, the multidomain therapeutic protein nucleic acid construct is delivered AAV-mediated delivery, and cleavage of the target sites flanking the nucleic acid insert can remove the inverted terminal repeats (ITRs) of the AAV. In some methods, the target site in the target genomic locus (e.g., a guide RNA target sequence including the flanking protospacer adjacent motif) is no longer present if the nucleic acid insert is inserted into the target genomic locus in the correct orientation but it is reformed if the nucleic acid insert is inserted into the target genomic locus in the opposite orientation.

The methods disclosed herein can comprise introducing or administering into a subject (e.g., an animal or mammal, such as a human) or cell a multidomain therapeutic protein nucleic acid construct and optionally a nuclease agent such as CRISPR/Cas reagents, including in the form of nucleic acids (e.g., DNA or RNA), proteins, or nucleic-acid-protein complexes. “Introducing” or “administering” includes presenting to the cell or subject the molecule(s) (e.g., nucleic acid(s) or protein(s)) in such a manner that it gains access to the interior of the cell or to the interior of cells within the subject. The introducing can be accomplished by any means, and two or more of the components (e.g., two of the components, or all of the components) can be introduced into the cell or subject simultaneously or sequentially in any combination. For example, a Cas protein can be introduced into a cell or subject before introduction of a guide RNA, or it can be introduced following introduction of the guide RNA. As another example, a multidomain therapeutic protein nucleic acid construct can be introduced prior to the introduction of a Cas protein and a guide RNA, or it can be introduced following introduction of the Cas protein and the guide RNA (e.g., the multidomain therapeutic protein nucleic acid construct can be administered about 1, 2, 3, 4, 8, 12, 24, 36, 48, or 72 hours before or after introduction of the Cas protein and the guide RNA). See, e.g., US 2015/0240263 and US 2015/0110762, each of which is herein incorporated by reference in its entirety for all purposes. In addition, two or more of the components can be introduced into the cell or subject by the same delivery method or different delivery methods. Similarly, two or more of the components can be introduced into a subject by the same route of administration or different routes of administration.

A guide RNA can be introduced into a subject or cell, for example, in the form of an RNA (e.g., in vitro transcribed RNA) or in the form of a DNA encoding the guide RNA. Guide RNAs can be modified as disclosed elsewhere herein. When introduced in the form of a DNA, the DNA encoding a guide RNA can be operably linked to a promoter active in the cell or in a cell in the subject. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Cas RNAs can be modified as disclosed elsewhere herein. Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or a subject, the Cas protein can be transiently, conditionally, or constitutively expressed in the cell or in a cell in the subject.

Nucleic acids encoding Cas proteins or guide RNAs can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding one or more gRNAs. Alternatively, it can be in a vector or plasmid that is separate from the vector comprising the DNA encoding one or more gRNAs. Suitable promoters that can be used in an expression construct include promoters active, for example, in one or more of a eukaryotic cell, a human cell, a non-human cell, a mammalian cell, a non-human mammalian cell, a rodent cell, a mouse cell, a rat cell, a hamster cell, a pluripotent cell, an embryonic stem (ES) cell, an adult stem cell, a developmentally restricted progenitor cell, an induced pluripotent stem (iPS) cell, or a one-cell stage embryo. For example, a suitable promoter can be active in a liver cell such as a hepatocyte. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allows for the generation of compact expression cassettes to facilitate delivery. In preferred embodiments, promotors are accepted by regulatory authorities for use in humans. In certain embodiments, promotors drive expression in a liver cell.

Molecules (e.g., Cas proteins or guide RNAs or nucleic acids encoding) introduced into the subject or cell can be provided in compositions comprising a carrier increasing the stability of the introduced molecules (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules.

Various methods and compositions are provided herein to allow for introduction of molecule (e.g., a nucleic acid or protein) into a cell or subject. Methods for introducing molecules into various cell types are known and include, for example, stable transfection methods, transient transfection methods, and virus-mediated methods.

Transfection protocols as well as protocols for introducing molecules into cells may vary. Non-limiting transfection methods include chemical-based transfection methods using liposomes; nanoparticles; calcium phosphate (Graham et al. (1973) Virology 52 (2): 456-67, Bacchetti et al. (1977) Proc. Natl. Acad. Sci. U.S.A. 74 (4):1590-4, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, sonoporation, and optical transfection. Particle-based transfection includes the use of a gene gun, or magnet-assisted transfection (Bertram (2006) Current Pharmaceutical Biotechnology 7, 277-28). Viral methods can also be used for transfection.

Introduction of nucleic acids or proteins into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In one example, nucleofection is performed using the LONZA® NUCLEOFECTOR™ system.

Introduction of molecules (e.g., nucleic acids or proteins) into a cell (e.g., a zygote) can also be accomplished by microinjection. In zygotes (i.e., one-cell stage embryos), microinjection can be into the maternal and/or paternal pronucleus or into the cytoplasm. If the microinjection is into only one pronucleus, the paternal pronucleus is preferable due to its larger size. Microinjection of an mRNA is preferably into the cytoplasm (e.g., to deliver mRNA directly to the translation machinery), while microinjection of a Cas protein or a polynucleotide encoding a Cas protein or encoding an RNA is preferable into the nucleus/pronucleus. Alternatively, microinjection can be carried out by injection into both the nucleus/pronucleus and the cytoplasm: a needle can first be introduced into the nucleus/pronucleus and a first amount can be injected, and while removing the needle from the one-cell stage embryo a second amount can be injected into the cytoplasm. If a Cas protein is injected into the cytoplasm, the Cas protein preferably comprises a nuclear localization signal to ensure delivery to the nucleus/pronucleus. Methods for carrying out microinjection are well known. See, e.g., Nagy et al. (Nagy A, Gertsenstein M, Vintersten K, Behringer R., 2003, Manipulating the Mouse Embryo. Cold Spring Harbor, New York: Cold Spring Harbor Laboratory Press); see also Meyer et al. (2010) Proc. Natl. Acad. Sci. U.S.A. 107:15022-15026 and Meyer et al. (2012) Proc. Natl. Acad. Sci. U.S.A. 109:9354-9359, each of which is herein incorporated by reference in its entirety for all purposes.

Other methods for introducing molecules (e.g., nucleic acid or proteins) into a cell or subject can include, for example, vector delivery, particle-mediated delivery, exosome-mediated delivery, lipid-nanoparticle-mediated delivery, cell-penetrating-peptide-mediated delivery, or implantable-device-mediated delivery. As specific examples, a nucleic acid or protein can be introduced into a cell or subject in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to a subject include hydrodynamic delivery, virus-mediated delivery (e.g., adeno-associated virus (AAV)-mediated delivery), and lipid-nanoparticle-mediated delivery.

Introduction of nucleic acids and proteins into cells or subjects can be accomplished by hydrodynamic delivery (HDD). For gene delivery to parenchymal cells, only essential DNA sequences need to be injected via a selected blood vessel, eliminating safety concerns associated with current viral and synthetic vectors. When injected into the bloodstream, DNA is capable of reaching cells in the different tissues accessible to the blood. Hydrodynamic delivery employs the force generated by the rapid injection of a large volume of solution into the incompressible blood in the circulation to overcome the physical barriers of endothelium and cell membranes that prevent large and membrane-impermeable compounds from entering parenchymal cells. In addition to the delivery of DNA, this method is useful for the efficient intracellular delivery of RNA, proteins, and other small compounds in vivo. See, e.g., Bonamassa et al. (2011) Pharm. Res. 28(4):694-701, herein incorporated by reference in its entirety for all purposes.

Introduction of nucleic acids can also be accomplished by virus-mediated delivery, such as AAV-mediated delivery or lentivirus-mediated delivery. Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression or longer-lasting expression. Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹² to about 10¹⁶ vg/mL. Other exemplary viral titers (e.g., AAV titers) include about 10¹² to about 10¹⁶ vg/kg of body weight.

Introduction of nucleic acids and proteins can also be accomplished by lipid nanoparticle (LNP)-mediated delivery. For example, LNP-mediated delivery can be used to deliver a combination of Cas mRNA and guide RNA or a combination of Cas protein and guide RNA. LNP-mediated delivery can be used to deliver a guide RNA in the form of RNA. In a specific example, the guide RNA and the Cas protein are each introduced in the form of RNA via LNP-mediated delivery in the same LNP. As discussed in more detail elsewhere herein, one or more of the RNAs can be modified. For example, guide RNAs can be modified to comprise one or more stabilizing end modifications at the 5′ end and/or the 3′ end. Such modifications can include, for example, one or more phosphorothioate linkages at the 5′ end and/or the 3′ end or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. As another example, Cas mRNA modifications can include substitution with N1-methyl-pseudouridine (e.g., fully substituted with N1-methyl-pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, 5024, 5027, 5031, or 5033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include a multidomain therapeutic protein nucleic acid construct. In certain LNPs, the cargo can include an mRNA encoding a Cas nuclease, such as Cas9, a guide RNA or a nucleic acid encoding a guide RNA, and a multidomain therapeutic protein nucleic acid construct. LNPs for use in the methods are described in more detail elsewhere herein.

The mode of delivery can be selected to decrease immunogenicity. For example, a Cas protein and a gRNA may be delivered by different modes (e.g., bi-modal delivery). These different modes may confer different pharmacodynamics or pharmacokinetic properties on the subject delivered molecule (e.g., Cas or nucleic acid encoding, gRNA or nucleic acid encoding, or multidomain therapeutic protein nucleic acid construct). For example, the different modes can result in different tissue distribution, different half-life, or different temporal distribution. Some modes of delivery (e.g., delivery of a nucleic acid vector that persists in a cell by autonomous replication or genomic integration) result in more persistent expression and presence of the molecule, whereas other modes of delivery are transient and less persistent (e.g., delivery of an RNA or a protein). Delivery of Cas proteins in a more transient manner, for example, as mRNA or protein, can ensure that the Cas/gRNA complex is only present and active for a short period of time and can reduce immunogenicity caused by peptides from the bacterially-derived Cas enzyme being displayed on the surface of the cell by MHC molecules. Such transient delivery can also reduce the possibility of off-target modifications.

Administration in vivo can be by any suitable route including, for example, systemic routes of administration such as parenteral administration, e.g., intravenous, subcutaneous, intra-arterial, or intramuscular. In a specific example, administration in vivo is intravenous.

Compositions comprising the guide RNAs and/or Cas proteins (or nucleic acids encoding the guide RNAs and/or Cas proteins) can be formulated using one or more physiologically and pharmaceutically acceptable carriers, diluents, excipients or auxiliaries. The formulation can depend on the route of administration chosen. Pharmaceutically acceptable means that the carrier, diluent, excipient, or auxiliary is compatible with the other ingredients of the formulation and not substantially deleterious to the recipient thereof. In a specific example, the route of administration and/or formulation or chosen for delivery to the liver (e.g., hepatocytes).

The methods disclosed herein can increase multidomain therapeutic protein or GAA protein levels and/or multidomain therapeutic protein or GAA activity levels in a cell or subject (e.g., circulating, serum, or plasma levels in a subject) and can comprise measuring multidomain therapeutic protein or GAA protein levels and/or multidomain therapeutic protein or GAA activity levels in a cell or subject (e.g., circulating, serum, or plasma levels in a subject). In one example, the methods result in increased expression of the multidomain therapeutic protein in the subject compared to a method comprising administering an episomal expression vector encoding the multidomain therapeutic protein or compared to a method in which a nucleic acid construct with a different polyadenylation signal is used. For example, the methods can result in increased serum levels of the multidomain therapeutic protein in the subject compared to a method comprising administering an episomal expression vector encoding the multidomain therapeutic protein or compared to a method in which a nucleic acid construct with a different polyadenylation signal is used. The methods can also result in increased multidomain therapeutic protein activity or GAA activity in the subject compared to a method comprising administering an episomal expression vector encoding the multidomain therapeutic protein or compared to a method in which a nucleic acid construct with a different polyadenylation signal is used. Levels of circulating multidomain therapeutic protein or GAA activity can be measured by using well-known methods.

In some methods, GAA activity and/or expression levels in a subject or in a target tissue (e.g., a target tissue in the central nervous system) are increased to about or at least about 2%, about or at least about 10%, about or at least about 25%, about or at least about 40%, about or at least about 50%, about or at least about 60%, about or at least about 70%, about or at least about 75%, about or at least about 80%, about or at least about 90%, or at least about 100%, or more, of normal level. In some methods, GAA activity and/or expression levels in a subject or in a target tissue (e.g., a target tissue in the central nervous system) are increased to about or at least about 40%, about or at least about 50%, about or at least about 60%, about or at least about 70%, about or at least about 75%, about or at least about 80%, about or at least about 90%, or at least about 100%, or more, of normal level. In certain embodiments, the level of expression or activity is measured in a cell or tissue in which a sign or symptom of the GAA loss of function is present. For example, when the loss of function results in muscle dysfunction, the level or activity of the multidomain therapeutic protein or GAA is measured in a muscle cell. It is understood that depending on the exogenous protein, the level of activity of the multidomain therapeutic protein may not compare 1:1 with a native GAA protein based on weight. In such embodiment, the relative activity of the multidomain therapeutic protein and the native GAA can be compared. In certain embodiments, the loss of function is nearly complete such that a relative activity cannot be determined. In certain embodiments, the comparison is made to an appropriate control subject. Selection of an appropriate control subject is within the ability of those of skill in the art. In certain embodiments, the level of expression is sufficient to treat at least one sign or symptom resulting from the loss of function of the GAA. GAA activity can be assessed by any known method. For example, to assess GAA activity (or deficiencies of activity), blood-based assays can measure GAA activity in dried blood spots or fresh blood. GAA activity can also be measured in fibroblasts from a skin biopsy or muscle biopsy. Other secondary measures can be measuring urine glucose tetrasaccharides by mass spectrometry.

In some methods, circulating multidomain therapeutic protein levels (i.e., serum levels) are about or at least about 0.5, about or at least about 1, about or at least about 2, about or at least about 3, about or at least about 4, about or at least about 5, about or at least about 6, about or at least about 7, about or at least about 8, about or at least about 9, or about or at least about 10 μg/mL. In some methods, multidomain therapeutic protein levels are at least about 1 μg/mL or about 1 μg/mL. In some methods, multidomain therapeutic protein levels are at least about 2 μg/mL or about 2 μg/mL. In some methods, multidomain therapeutic protein levels are at least about 3 μg/mL or about 3 μg/mL. In some methods, multidomain therapeutic protein levels are at least about 5 μg/mL or about 5 μg/mL. In some methods, multidomain therapeutic protein levels are about 1 μg/mL to about 30 μg/mL, about 2 μg/mL to about 30 μg/mL, about 3 μg/mL to about 30 μg/mL, about 4 μg/mL to about 30 μg/mL, about 5 μg/mL to about 30 μg/mL, about 1 μg/mL to about 20 μg/mL, about 2 μg/mL to about 20 μg/mL, about 3 μg/mL to about 20 μg/mL, about 4 μg/mL to about 20 μg/mL, about 5 μg/mL to about 20 μg/mL. For example, the method can result in multidomain therapeutic protein levels of about 2 μg/mL to about 30 μg/mL or 2 μg/mL to about 20 μg/mL. For example, the method can result in multidomain therapeutic protein levels of about 3 μg/mL to about 30 μg/mL or 3 μg/mL to about 20 μg/mL. For example, the method can result in multidomain therapeutic protein levels of about 5 μg/mL to about 30 μg/mL or 5 μg/mL to about 20 μg/mL. In some embodiments, the recited expression levels are at least 1 month after administration. In some embodiments, the recited expression levels are at least 2 months after administration. In some embodiments, the recited expression levels are at least 3 months after administration. In some embodiments, the recited expression levels are at least 4 months after administration. In some embodiments, the recited expression levels are at least 5 months after administration. In some embodiments, the recited expression levels are at least 6 months after administration. In some embodiments, the recited expression levels are at least 9 months after administration. In some embodiments, the recited expression levels are at least 12 months after administration.

In some methods, the method increases expression and/or activity of GAA or the multidomain therapeutic protein over the subject's baseline expression and/or activity (i.e., expression and/or activity prior to administration). In some methods, the method increases expression and/or activity of GAA over the subject's baseline expression and/or activity (i.e., expression and/or activity prior to administration. In some methods, GAA activity and/or GAA expression or serum levels in a subject are increased by about or at least about 10%, about or at least about 25%, about or at least about 50%, about or at least about 75%, or about or at least about 100%, or more, as compared to the subject's GAA expression or serum levels and/or activity (e.g., GAA activity) before administration (i.e., the subject's baseline levels). It is understood that depending on the multidomain therapeutic protein, the level of activity of the multidomain therapeutic protein may not compare 1:1 with a native protein based on weight. In such embodiment, the relative activity of the multidomain therapeutic protein and the native GAA can be compared. In certain embodiments, the loss of function is nearly complete such that a relative activity cannot be determined. In certain embodiments, the level of expression is sufficient to treat at least one sign or symptom resulting from the loss of function of the GAA.

In some methods, the method increases expression and/or activity of the multidomain therapeutic protein over the cell's baseline expression and/or activity (i.e., expression and/or activity prior to administration). In some methods, the method increases expression and/or activity of GAA over the cell's baseline expression and/or activity (i.e., expression and/or activity prior to administration. In some methods, GAA activity and/or expression levels in a cell or population of cells (e.g., liver cells, or hepatocytes) are increased by about or at least about 10%, about or at least about 25%, about or at least about 50%, about or at least about 75%, about or at least about 100%, or more, as compared to the GAA activity and/or expression levels before administration (i.e., the subject's baseline levels). It is understood that depending on the multidomain therapeutic protein, the level of activity of the multidomain therapeutic protein may not compare 1:1 with a native GAA protein based on weight. In such embodiment, the relative activity of the multidomain therapeutic protein and the native GAA protein can be compared. In certain embodiments, the GAA loss of function is nearly complete such that a relative activity cannot be determined. In certain embodiments, the level of expression is sufficient to treat at least one sign or symptom resulting from the loss of function of the GAA.

In a specific example, the GAA activity levels in a subject or in a target tissue (e.g., a target tissue in the central nervous system) are increased to no more than about 500%, no more than about 400%, no more than about 300%, no more than about 250%, no more than about 200%, or no more than about 150% of normal GAA activity levels.

In a specific example, the GAA activity levels in the subject or in a target tissue (e.g., a target tissue in the central nervous system) are increased to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels. In a specific example, the GAA activity levels in the subject or in a target tissue (e.g., a target tissue in the central nervous system) are increased to at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels.

In a specific example, a subject has infantile-onset Pompe disease (e.g., classic infantile-onset Pompe disease), and the GAA activity levels in the subject are increased to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels. In a specific example, a subject has infantile-onset Pompe disease (e.g., classic infantile-onset Pompe disease), and the GAA activity levels in the subject are increased to at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels.

In a specific example, a subject has infantile-onset Pompe disease (e.g., classic or non-classic infantile-onset Pompe disease), and the GAA activity levels in the subject are increased to at least about 2% at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels (e.g., at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels). In a specific example, a subject has infantile-onset Pompe disease (e.g., classic or non-classic infantile-onset Pompe disease), and the GAA activity levels in the subject are increased to at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels.

In a specific example, a subject has late-onset Pompe disease, and the GAA activity levels in the subject are increased to at least about 2% at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels (e.g., at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels).

In a specific example, a subject has infantile-onset Pompe disease (e.g., classic infantile-onset Pompe disease), and the GAA activity levels in the subject are increased to more than about 1%, more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels. In a specific example, a subject has infantile-onset Pompe disease (e.g., classic infantile-onset Pompe disease), and the GAA activity levels in the subject are increased to more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels

In a specific example, a subject has infantile-onset Pompe disease (e.g., classic or non-classic infantile-onset Pompe disease), and the GAA activity levels in the subject are increased to more than about 2% more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels (e.g., more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels). In a specific example, a subject has infantile-onset Pompe disease (e.g., classic or non-classic infantile-onset Pompe disease), and the GAA activity levels in the subject are increased to more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels.

In a specific example, a subject has late-onset Pompe disease, and the GAA activity levels in the subject are increased to more than about 2% more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels (e.g., more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels).

In some methods, the method results in increased expression of the multidomain therapeutic protein in the subject (e.g., neonatal subject) compared to a method comprising administering an episomal expression vector encoding the multidomain therapeutic protein in a control subject or compared to a method in which a nucleic acid construct with a different polyadenylation signal is used. In some methods, the method results in increased serum levels of the multidomain therapeutic protein in the subject (e.g., neonatal subject) compared to a method comprising administering an episomal expression vector encoding the multidomain therapeutic protein to a control subject or compared to a method in which a nucleic acid construct with a different polyadenylation signal is used.

In some methods, the method increases expression or activity of the multidomain therapeutic protein or GAA over the subject's (e.g., neonatal subject's) baseline expression or activity of the multidomain therapeutic protein or GAA (i.e., any percent change in expression that is larger than typical error bars). In some methods, the method results in expression of the multidomain therapeutic protein or GAA at a detectable level above zero, e.g., at a statistically significant level, a clinically relevant level.

Some methods comprise achieving a durable or sustained effect in a human, such as an at least at least 8 weeks, at least 24 weeks, for example, at least 1 year, or optionally at least 2 year effect, and in some embodiments, at least 3 year, at least 4 year, or at least 5 year effect. Some methods comprise achieving the therapeutic effect in a human in a durable and sustained manner, such as an at least 8 weeks, at least 24 weeks, for example, at least 1 year, or optionally at least 2 year effect, and in some embodiments, at least 3 year, at least 4 year, or at least 5 year effect. In some methods, the increased multidomain therapeutic protein or GAA activity and/or expression level in a human is stable for at least at least 8 weeks, at least 24 weeks, for example, at least 1 year, optionally at least 2 years, and in some embodiments, at least 3 years, at least 4 years, or at least 5 years. In some methods, a steady-state activity and/or level of multidomain therapeutic protein or GAA in a human is achieved by at least 7 days, at least 14 days, or at least 28 days, optionally at least 56 days, at least 80 days, or at least 96 days. In additional methods, the method comprises maintaining multidomain therapeutic protein or GAA activity and/or levels after a single dose in a human for at least 8 weeks, at least 16 weeks, or at least 24 week, or in some embodiments at least 1 year, or at least 2 years, optionally at least 3 years, at least 4 years, or at least 5 years. For example, expression of the multidomain therapeutic protein or GAA can be sustained in the human subject for at least about 8 weeks, at least about 12 weeks, at least about 24 weeks, in certain embodiments, at least about 1 year, or at least about 2 years after treatment, and in some embodiments, at least 3 years, at least 4 years, or at least 5 years after treatment. Likewise, activity of the multidomain therapeutic protein or GAA can be sustained in the human subject for at least about 8 weeks, at least about 12 weeks, at least about 24 weeks, in certain embodiments for at least about 1 year, or at least about 2 years after treatment, and in some embodiments, at least 3 years, at least 4 years, or at least 5 years after treatment. In some methods, expression or activity of the multidomain therapeutic protein or GAA is maintained at a level higher than the expression or activity of the multidomain therapeutic protein or GAA prior to treatment (i.e., the subject's baseline). In some methods, expression or activity of the multidomain therapeutic protein or GAA is considered sustained if it is maintained at a therapeutically effective level of expression or activity. Relative durations, in other organisms, are understood based, e.g., on life span and developmental stages, are covered within the disclosure above. In some methods, expression or activity of the multidomain therapeutic protein or GAA is considered “sustained” if the expression or activity in a human at six months after administration, one year after administration, or two years after administration, the expression or activity is at least 50% of the expression or activity of the peak level of expression or activity measured for that subject. In certain embodiments, at six months, e.g., at 24 weeks to 28 weeks, after administration the expression or activity is at least 50%, 55%, 60%, 65%, 70%, 75% or 80% of the expression or activity of the peak level of expression or activity measured for that subject. In certain embodiments, at one year, i.e., about 12 months, e.g., at 11-13 months, after administration the expression or activity is at least 50%, 55%, 60%, 65%, 70%, 75% or 80% of the expression or activity of the peak level of expression or activity measured for that subject. In certain embodiments, at two years, i.e., about 24 months, e.g., at 23-25 months, after administration the expression or activity is at least 50%, 55%, 60%, 65%, 70%, 75% or 80% of the expression or activity of the peak level of expression or activity measured for that subject. In certain embodiments, at six months after administration the expression or activity is at least 50%, preferably at least 60% of the expression or activity of the peak level of expression or activity measured for that subject. In certain embodiments, at one year after administration the expression or activity is at least 50%, preferably at least 60% of the expression or activity of the peak level of expression or activity measured for that subject. In certain embodiments, at two years after administration the expression or activity is at least 50%, preferably at least 60% of the expression or activity of the peak level of expression or activity measured for that subject. In preferred embodiments, the subject has routine monitoring of expression or activity levels of the polypeptide, e.g., weekly, monthly, particularly early after administration, e.g., within the first six months. Periodic measurements may establish that the effect on expression or activity is sustained at, e.g., 6 months after administration, one year after administration, or two years after administration. In some methods in neonatal subjects, the expression of the multidomain therapeutic protein or GAA is sustained when the neonatal subject becomes an adult. In some methods, the expression of the multidomain therapeutic protein or GAA is sustained for the lifetime of the subject or neonatal subject.

In some methods, the expression or activity of the multidomain therapeutic protein is at least 50% of the expression or activity of the multidomain therapeutic protein at a peak level of expression measured for the subject at 24 weeks after the administering. In some methods, the expression or activity of the multidomain therapeutic protein is at least 50% of the expression or activity of the multidomain therapeutic protein at a peak level of expression measured for the subject at one year after the administering. In some methods, the expression or activity of the multidomain therapeutic protein is at least 60% of the expression or activity of the multidomain therapeutic protein at a peak level of expression measured for the subject at 24 weeks after the administering. In some methods, the expression or activity of the multidomain therapeutic protein is at least 50% of the expression or activity of the multidomain therapeutic protein at a peak level of expression measured for the subject at two years after the administering. In some methods, the expression or activity of the multidomain therapeutic protein is at least 60% of the expression or activity of the multidomain therapeutic protein at a peak level of expression measured for the subject at 2 years after the administering. In some methods, the expression or activity of the multidomain therapeutic protein is at least 60% of the expression or activity of the multidomain therapeutic protein at a peak level of expression measured for the subject at 24 weeks after the administering.

In some methods involving insertion into an ALB locus, the subject's circulating albumin levels or cell's albumin levels are normal. Such methods may comprise maintaining the subject's circulating albumin levels or the cell's albumin levels within +5%, +10%, +15%, ±20%, or ±50% of normal circulating albumin levels or normal albumin levels. In some methods, the subject's or cell's albumin levels are unchanged as compared to the albumin levels of untreated individuals by at least week 4, at least week 8, at least week 12, or at least week 20. In some methods, the subject's or cell's albumin levels transiently drop and then return to normal levels. In particular, the methods may comprise detecting no significant alterations in levels of plasma albumin.

In some methods, the method further comprises assessing preexisting anti-GAA immunity in a subject prior to administering any of the nucleic acid constructs described herein. For example, such methods could comprise assessing immunogenicity using a total antibody (TAb) immune assay or a neutralizing antibody (NAb) assay. In some methods, the subject has not previously been administered recombinant GAA protein.

In some methods, the method further comprises assessing preexisting anti-AAV (e.g., anti-AAV8) immunity in a subject prior to administering any of the nucleic acid constructs described herein. For example, such methods could comprise assessing immunogenicity using a total antibody (TAb) immune assay or a neutralizing antibody (NAb) assay. See, e.g., Manno et al. (2006) Nat. Med. 12(3):342-347, Kruzik et al. (2019) Mol. Ther. Methods Clin. Dev. 14:126-133, and Weber (2021) Front. Immunol. 12:658399, each of which is herein incorporated by reference in its entirety for all purposes. In some embodiments, TAb assays look for antibodies that bind to the AAV vector, whereas NAb assays assess whether the antibodies that are present stop the AAV vector from transducing target cells. With TAb assays, the drug product or an empty capsid can be used to capture the antibodies; NAb assays can require a reporter vector (e.g., a version of the AAV vector encoding luciferase).

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanting, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 11
Description of Sequences.

| SEQ ID | | |
|---|---|---|
| NO | Type | Description |
| 1 | RNA | Cas9 mRNA |
| 2 | RNA | Cas9 mRNA CDS |
| 3 | DNA | Cas9 CDS |
| 4 | DNA | Human ALB Intron 1 |
| 5 | DNA | Guide RNA Target Sequence Plus PAM v1 |
| 6 | DNA | Guide RNA Target Sequence Plus PAM v2 |
| 7 | DNA | Guide RNA Target Sequence Plus PAM v3 |
| 8 | Protein | SpCas9 Protein V1 |
| 9 | DNA | SpCas9 DNA V1 |
| 10 | DNA | SpCas9 mRNA (cDNA) |
| 11 | Protein | SpCas9 Protein V2 |
| 12 | RNA | SpCas9 mRNA V2 |
| 13 | Protein | SV40 NLS v1 |
| 14 | Protein | SV40 NLS v2 |
| 15 | Protein | Nucleoplasmin NLS |
| 16 | RNA | crRNA Tail v1 |
| 17 | RNA | crRNA Tail v2 |
| 18 | RNA | TracrRNA v1 |
| 19 | RNA | TracrRNA v2 |
| 20 | RNA | TracrRNA v3 |
| 21 | RNA | gRNA Scaffold v1 |
| 22 | RNA | gRNA Scaffold v2 |
| 23 | RNA | gRNA Scaffold v3 |
| 24 | RNA | gRNA Scaffold v4 |
| 25 | RNA | gRNA Scaffold v5 |
| 26 | RNA | gRNA Scaffold v6 |
| 27 | RNA | gRNA Scaffold v7 |
| 28 | RNA | gRNA Scaffold v8 |
| 29 | RNA | Modified gRNA Scaffold |
| 30-61 | RNA | Human ALB Intron 1 Guide Sequences |
| 62-125 | RNA | Human ALB Intron 1 sgRNA Sequences |
| 126-157 | DNA | Human ALB Intron 1 Guide RNA Target Sequences |
| 158 | DNA | ITR 145 |
| 159 | DNA | ITR 141 |
| 160 | DNA | ITR 130 |
| 161 | DNA | SV40 polyA |
| 162 | DNA | bGH polyA |
| 163 | DNA | Mouse Alb exon 2 Splice Acceptor |
| 164 | RNA | Mouse Alb Intron 1 Guide Sequence g666 |
| 165 | DNA | Mouse Alb Intron 1 Guide RNA Target Sequence g666 |
| 166-167 | RNA | Mouse Alb Intron 1 sgRNA Sequences g666 |
| 168 | DNA | ITR 145 Reverse Complement |
| 169 | DNA | SV40 polyA v2 |
| 170-491 | DNA & Protein | Domains in Anti-hTfR Antibodies, Antigen-Binding Fragments or scFv Molecules |
| 492-523 | Protein | Anti-hTfR scFvs |
| 524 | DNA | 12799 GA 0 anti-TfR scFv |
| 525 | DNA | 12799 GS 0 anti-TfR scFv |
| 526 | DNA | 12799 GS Ov2 anti-TfR scFv |
| 527 | DNA | 12843 GA 0 anti-TfR scFv |
| 528 | DNA | 12843 GS 0 anti-TfR scFv |
| 529 | DNA | 12843 GS Ov2 anti-TfR scFv |
| 530 | DNA | 12847 GA 0 anti-TfR scFv |
| 531 | DNA | 12847 GS 0 anti-TfR scFv |
| 532 | DNA | 12847 GS Ov2 anti-TfR scFv |
| 533 | DNA | 12799 anti-TfR scFv |
| 534 | DNA | 12839 anti-TfR scFv |
| 535 | DNA | 12843 anti-TfR scFv |
| 536 | DNA | 12847 anti-TfR scFv |
| 537 | Protein | Linker |
| 538 | Protein | Kappa Constant Light Domain |
| 539 | Protein | IgG1 CH1 Heavy Domain |
| 540-609 | Protein | Fab Heavy and Light Chains |
| 610 | Protein | Mus musculus Ror1 Signal Peptide |
| 611 | Protein | IgG4 CH1 Heavy Domain |
| 612 | Protein | Optional N-Terminal Sequence |
| 613 | DNA | ITR 141 Reverse Complement |
| 614 | DNA | ITR 130 Reverse Complement |
| 615 | DNA | SV40 polyA V3 |
| 616 | Protein | 3X G4S Linker |
| 617 | Protein | 2X G4S Linker |
| 618-622 | DNA | 3X G4S Linker Coding Sequences |
| 623-629 | DNA | 2X G4S Linker Coding Sequences |
| 630 | DNA | 1X G4S Linker Coding Sequence |
| 631-638 | Protein | Additional Anti-hTfR Antibody Sequences |
| 639-641 | Protein | Sequences from Examples |
| 642-643 | DNA | Sequences from Examples |
| 644-647 | Protein | Sequences from Examples |
| 648-679 | DNA | Sequences from Examples |
| 680-703 | Protein | Additional Anti-hTfR Antibody Sequences |
| 704-723 | Protein | TfR Epitopes |
| 724 | Protein | Human GAA Protein (NP_000143.2) |
| 725 | DNA | Human GAA cDNA/mRNA (NM_000152.5) |
| 726 | DNA | Human GAA CDS (CCDS32760.1) |
| 727 | Protein | Human GAA (70-952) Protein |
| 728 | DNA | Human GAA (70-952) CDS |
| 729 | DNA | Human GAA (70-952) CDS - GA-0 |
| 730 | Protein | 12450 anti-CD63 scFv |
| 731 | DNA | 12450 anti-CD63 scFv CDS |
| 732 | DNA | 12450 anti-CD63 scFv CDS - GA-0 |
| 733 | Protein | 12450 anti-CD63 scFv:GAA (70-952) Fusion Protein |
| 734 | DNA | 12450 anti-CD63 scFv:GAA (70-952) Fusion Protein CDS |
| 735 | DNA | 12450 anti-CD63 scFv:GAA (70-952) Fusion Protein CDS - DC-0 |
| 736 | DNA | 12450 anti-CD63 scFv:GAA (70-952) Fusion Protein CDS - GA-0 |
| 737 | DNA | 12450 anti-CD63 scFv:GAA (70-952) Fusion Protein CDS - GS-0 |
| 738 | DNA | 12450 anti-CD63 scFv:GAA (70-952) Fusion Protein CDS - GS-0v2 |
| 739 | DNA | 12450 anti-CD63 scFv:GAA (70-952) Fusion Protein CDS - GS-1 |
| 740 | DNA | 12450 anti-CD63 scFv:GAA (70-952) Fusion Protein CDS - GS-44 |
| 741 | DNA | 12450 anti-CD63 scFv:GAA (70-952) Fusion Protein CDS - GS-50 |
| 742 | DNA | 12450 anti-CD63 scFv:GAA (70-952) Fusion Protein CDS - RE-8 |
| 743 | DNA | Glycine-Encoding Sequence v1 |
| 744 | DNA | Glycine-Encoding Sequence v2 |
| 745 | DNA | 2xFix 12847 anti-TfR:GAA Coding Sequence |
| 746 | Protein | 12847 anti-TfR:GAA Fusion Protein |
| 747 | DNA | 3xG4S Linker Coding Sequence |
| 748 | DNA | 1xG4S Linker Coding Sequence |
| 749 | DNA | GAA (70-952) Coding Sequence [3078_GT_>_GG] |
| 750 | DNA | GAA (70-952) Coding Sequence [1830_GGTGGT_>_GGGGGC] |
| | | [3078_GT_>_GG] |
| 751 | DNA | bGH polyA v2 |
| 752 | DNA | SV40_late_polyA w/Reverse Strand AAUAAAs Mutated to AAUCAAs |
| 753 | DNA | Synthetic polyA (SPA) |
| 754 | DNA | Stuffer Sequence |
| 755 | DNA | MAZ Element |
| 756 | DNA | 6xFix anti-CD63:GAA Coding Sequence |
| 757 | DNA | 4xFix anti-CD63:GAA Coding Sequence [274_G > T] 723_T > G |
| | | 1830_GGT_GGT_>_GGG_GGC 3078_GT > GG] |
| 758 | DNA | 1xFix anti-CD63:GAA Coding Sequence [3078_GT > GG] |
| 759 | DNA | 6xFix anti-CD63 scFv Coding Sequence |
| 760 | DNA | 4xFix anti-CD63 scFv Coding Sequence |
| 761 | Protein | Anti-CD63 scFv VH |
| 762 | Protein | Anti-CD63 scFv VK |
| 763 | DNA | VVT874/VVT1251 - TfR Original Template pMID27199 ITR to ITR |
| 764 | DNA | VVT1125/VVT1261 - pAAV-12847GAA(GS0v2)-2Xfix-bGH.sv40LuniPA |
| 765 | DNA | VVT1129/VVT1262 - pAAV-12847GAA(GS0v2)-2Xfix-bGHpA |
| 766 | DNA | VVT1126 - pAAV-12847GAA(GS0v2)-bGHpA swap |
| 767 | DNA | VVT1254/VVT1252 - CD63 Original Template pMID27863 ITR to ITR |
| 768 | DNA | VVT1121 - pAAV-CD63GAA-4Xfix-bGH.sv40LuniPA |
| 769 | DNA | VVT1122 - pAAV-CD63GAA-4Xfix-bGHpA |
| 770 | DNA | VVT1123 - pAAV-CD63GAA-3078fix-bGH.sv40LuniPA (1 CpG) |
| 771 | DNA | VVT1118 - pAAV-CD63GAA-3078fix-bGHpA |
| 772 | DNA | VVT1124 - pAAV-CD63GAA-bGHpA swap |
| 773 | DNA | VVT1119 - pAAV-CD63GAA-bGHpA.SPA swap |
| 774 | DNA | VVT1120 - pAAV-CD63GAA-bGHpA.stuffer.SPA |
| 775 | DNA | VVT1138 - pAAV-CD63GAA-bGHpA-MAZ |
| 776 | DNA | VVT1127 - pAAV-CD63GAA-SV40E-MAZ |
| 777 | DNA | VVT1128/VVT1263 - pAAV-CD63GAA-6Xfix-bGH.sv40LuniPA |
| 778 | DNA | VVT1139/VVT1264 - pAAV-CD63GAA-6Xfix-bGHpA |
| 779 | DNA | VVT874/VVT1251 - TfR Original Template pMID27199 - no ITRs |
| 780 | DNA | VVT1125VVT/1261 - pAAV-12847GAA(GS0v2)-2Xfix-bGH.sv40LuniPA - no |
| | | ITRs |
| 781 | DNA | VVT1129/VVT1262 - pAAV-12847GAA(GS0v2)-2Xfix-bGHpA - no ITRs |
| 782 | DNA | VVT1126 - pAAV-12847GAA(GS0v2)-bGHpA swap - no ITRs |
| 783 | DNA | VVT1254/VVT1252 - CD63 Original Template pMID27863 - no ITRs |
| 784 | DNA | VVT1121 - pAAV-CD63GAA-4Xfix-bGH.sv40LuniPA - no ITRs |
| 785 | DNA | VVT1122 - pAAV-CD63GAA-4Xfix-bGHpA - no ITRs |
| 786 | DNA | VVT1123 - pAAV-CD63GAA-3078fix-bGH.sv40LuniPA (1 CpG) - no ITRs |
| 787 | DNA | VVT1118 - pAAV-CD63GAA-3078fix-bGHpA - no ITRs |
| 788 | DNA | VVT1124 - pAAV-CD63GAA-bGHpA swap - no ITRs |
| 789 | DNA | VVT1119 - pAAV-CD63GAA-bGHpA.SPA swap - no ITRs |
| 790 | DNA | VVT1120 - pAAV-CD63GAA-bGHpA.stuffer.SPA - no ITRs |
| 791 | DNA | VVT1138 - pAAV-CD63GAA-bGHpA-MAZ - no ITRs |
| 792 | DNA | VVT1127 - pAAV-CD63GAA-SV40E-MAZ - no ITRs |
| 793 | DNA | VVT1128/VVT1263 - pAAV-CD63GAA-6Xfix-bGH.sv40LuniPA - no ITRs |
| 794 | DNA | VVT1139/VVT1264 - pAAV-CD63GAA-6Xfix-bGHpA - no ITRs |
| 795 | DNA | Combined BGH and Unidirectional SV40 Late Polyadenylation Signal |
| 796-806 | Protein | TfR Epitopes |
| 807 | Protein | Heavy Chain Constant Domain |
| 808-839 | Protein | HC Full hlgG1 Sequences |
| 840 | Protein | IgG1 Heavy Chain Constant Domain |
| 841 | DNA | 2XH4 Linker CDS |
| 842 | Protein | 2XH4 Linker |

### EXAMPLES

### Example 1. Development of System for Insertion into Albumin Locus in Liver

A system for nuclease-mediated insertion (e.g., CRISPR/Cas) of a transgene into a specific locus (e.g., albumin intron 1) was developed to produce durable expression of the transgene, including when administered to neonates. Exemplary components of the system, including those used in subsequent examples, are described in more detail below.

### Single Guide RNA Design and Selection

The ALB locus was selected as the insertion site for the DNA templates. A list of single guide RNAs (sgRNAs) was generated that target human ALB intron 1. See Table 12. Cantidate sgRNAs were synthesized and formulated into lipid nanoparticles (LNPs) with Cas9 mRNA for evaluation in vitro and in vivo.

TABLE 12
Human ALB Intron 1 Guide RNAs.

| | SEQ ID | SEQ ID | SEQ ID | SEQ ID |
|---|---|---|---|---|
| | NO (DNA- | NO (Un- | NO | NO (Guide |
| Guide | Targeting | modified | (Modified | RNA Target |
| RNA | Segment) | sgRNA) | sgRNA) | Sequence) |
| G009844 | 30 | 62 | 94 | 126 |
| G009851 | 31 | 63 | 95 | 127 |
| G009852 | 32 | 64 | 96 | 128 |
| G009857 | 33 | 65 | 97 | 129 |
| G009858 | 34 | 66 | 98 | 130 |
| G009859 | 35 | 67 | 99 | 131 |
| G009860 | 36 | 68 | 100 | 132 |
| G009861 | 37 | 69 | 101 | 133 |
| G009866 | 38 | 70 | 102 | 134 |
| G009867 | 39 | 71 | 103 | 135 |
| G009868 | 40 | 72 | 104 | 136 |
| G009874 | 41 | 73 | 105 | 137 |
| G012747 | 42 | 74 | 106 | 138 |
| G012748 | 43 | 75 | 107 | 139 |
| G012749 | 44 | 76 | 108 | 140 |
| G012750 | 45 | 77 | 109 | 141 |
| G012751 | 46 | 78 | 110 | 142 |
| G012752 | 47 | 79 | 111 | 143 |
| G012753 | 48 | 80 | 112 | 144 |
| G012754 | 49 | 81 | 113 | 145 |
| G012755 | 50 | 82 | 114 | 146 |
| G012756 | 51 | 83 | 115 | 147 |
| G012757 | 52 | 84 | 116 | 148 |
| G012758 | 53 | 85 | 117 | 149 |
| G012759 | 54 | 86 | 118 | 150 |
| G012760 | 55 | 87 | 119 | 151 |
| G012761 | 56 | 88 | 120 | 152 |
| G012762 | 57 | 89 | 121 | 153 |
| G012763 | 58 | 90 | 122 | 154 |
| G012764 | 59 | 91 | 123 | 155 |
| G012765 | 60 | 92 | 124 | 156 |
| G012766 | 61 | 93 | 125 | 157 |

LNPs were first screened in primary human hepatocytes (PHH) using a bidirectional nanoluc-encoding AAV insertion template as a reporter. LNPs that supported targeted insertion of nanoluc were identified by measuring nanoluc protein secreted into the supernatant of PHH cultures. Cantidates that passed initial PHH screening were then tested for their ability to support in vivo gene insertion. Top cantidates from in vivo studies were functionally evaluated for off-target cutting.

LNP-g9860, which is formulated with ALB-targeting sgRNA 9860, described in more detail below, was selected based on supporting robust transgene expression levels across multiple platforms (primary human and non-human primate hepatocytes, ALB humanized mice, and non-human primates), lack of confirmed off-target sites, translation across species, lack of common human SNPs in the target site, low variability of transgene expression within groups, and performance across a dose range. The target site of sgRNA 9860 is conserved in cynomolgus monkeys. LNP-g9860 had no detectable off-target sites in the human genome (targeted amplicon sequencing performed in two lots of primary human hepatocytes at saturating levels of editing failed to validate any locus other than on-target at ALB) and supported transgene expression via insertion in primary human and non-human primate hepatocytes, ALB humanized mice, and non-human primates.

### LNP-g9860

LNP-g9860 was developed for use in targeting human ALB intron 1. LNP-g9860 is a lipid nanoparticle that includes a sgRNA of 100 nucleotides in length (g9860) and Cas9-encoding mRNA, each of which is described further below, encapsulated in an LNP comprised of four different lipids. The Cas9 protein, expressed from the Cas9 mRNA, is directed to cleave the DNA when sgRNA 9860 binds to the targeted complementary DNA sequence associated with a PAM. The composition of the LNP is summarized in Table 13. LNP-g9860 comprises four lipids at the following molar ratios: 50 mol % Lipid A, 9 mol % DSPC, 38 mol % cholesterol, and 3 mol % PEG2k-DMG and is formulated in aqueous buffer composed of 50 mM Tris-HCl, 45 mM NaCl, 5% (w/v) sucrose, at pH 7.4. The N:P ratio is about 6, and the gRNA:Cas9 mRNA ratio is about 1:2 by weight.

TABLE 13
Lipid Nanoparticle (LNP-g9860) Composition.

| Component | Description |
|---|---|
| Active Pharmaceutical Components | Cas9 mRNA |
| | sgRNA (gRNA9860) |
| Lipid Excipients | Lipid A: (9Z,12Z)-3-((4,4- |
| | bis(octyloxy)butanoyl)oxy)-2-((((3- |
| | (diethylamino)propoxy)carbonyl)oxy)methyl)propyl |
| | octadeca-9,12-dienoate |
| | Cholesterol |
| | DSPC |
| | PEG2K-DMG |
| Other Excipients | Tris, NaCl, Sucrose |
| | WFI |

Single guide RNA. The single guide RNA (sgRNA 9860) used in LNP-g9860 is a 100-mer oligonucleotide containing a 20-nucleotide sequence that is complementary to the target region in intron 1 of the human ALB gene. The target sequence recognized by g9860 is conserved in the cynomolgus monkey mfAlb gene intron 1. The sequence for g9860 is set forth in SEQ ID NOs: 68 and 100. Chemical modifications are incorporated into the 100-mer during synthesis, which include phosphorothioate (PS) linkages at the 5′- and 3′-end of the sgRNA and 2′-O-methyl modifications to some of the sugars of the RNA.

Cas9 mRNA. The Cas9 messenger RNA (mRNA) used in LNP-g9860 is based on the Cas9 protein sequence from Streptococcus pyogenes. The Cas9-encoding mRNA (SEQ ID NO: 1, with a coding sequence (CDS) set forth in SEQ ID NO: 2), is approximately 4400 nucleotides in length. The sequence contains a 5′ cap, a 5′ untranslated region (UTR), an open reading frame (ORF) encoding the Cas9 protein, a 3′ UTR, and a polyA tail. The 5′ cap is generated co-transcriptionally by use of a synthetic cap analogue structure, known as anti-reverse cap analogue (ARCA). The uracils in the mRNA sequence have been completely replaced by a modified N¹ methylpseudouridine during the in vitro transcription. The 5′ end of the mRNA has a synthetic cap analog structure. The poly-A tail is approximately 100 nucleotides.

### LNP-g666

LNP-g666 was developed for use in targeting mouse Alb intron 1. LNP-g666 is the same as LNP-g9860, except human-albumin-targeting g9860 is replaced with g666, a guide RNA targeting mouse albumin intron 1. The sequence for g666 is set forth in SEQ ID NOS: 166 and 167.

rAAV8 Vector

A recombinant AAV8 (rAAV8) vector was developed to carry the DNA insertion templates. The rAAV8 vector carrying the DNA insertion templates is a non-replicating vector that is an AAV-based vector derived from AAV serotype 8. The genome is a single-stranded deoxyribonucleic acid (DNA), comprising inverted terminal repeats (ITR) at each end. The ITRs flank the promoterless insertion template. The AAV ITRs flanking the cassette were derived from AAV2. The DNA insertion templates delivered by rAAV8 vector can be designed as promoterless templates, thus relying on the targeted ALB locus promoter for expression.

### Example 2. Development of Insertion System and Reagents for Treatment of Pompe Disease

A system for nuclease-mediated insertion (e.g., CRISPR/Cas) of an anti-CD63:GAA transgene or an anti-TfR:GAA transgene into a specific locus (e.g., albumin intron 1) was developed to produce durable expression of anti-CD63:GAA or anti-TfR:GAA, including when administered to neonates.

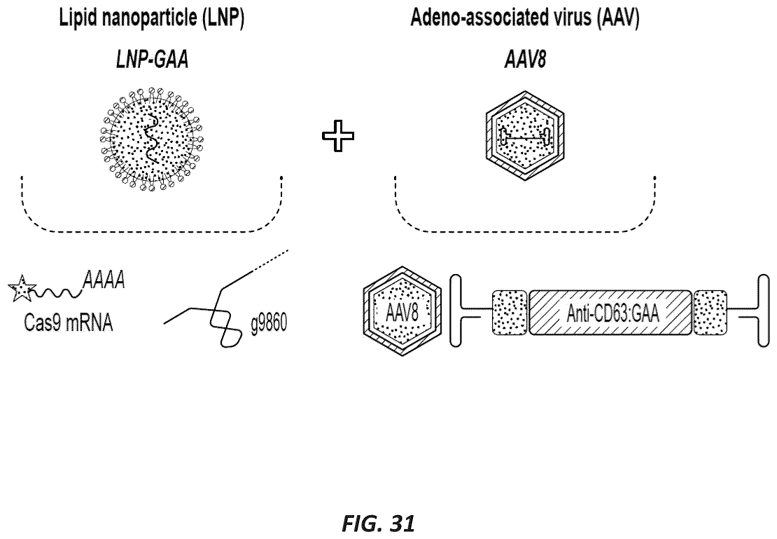

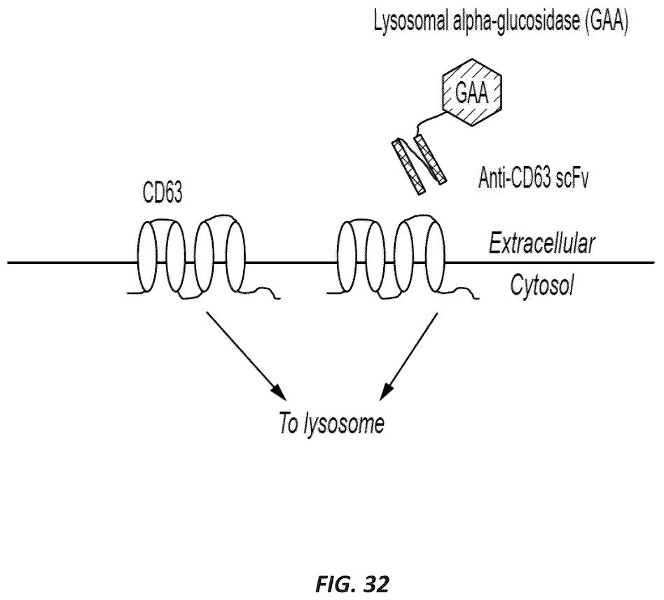

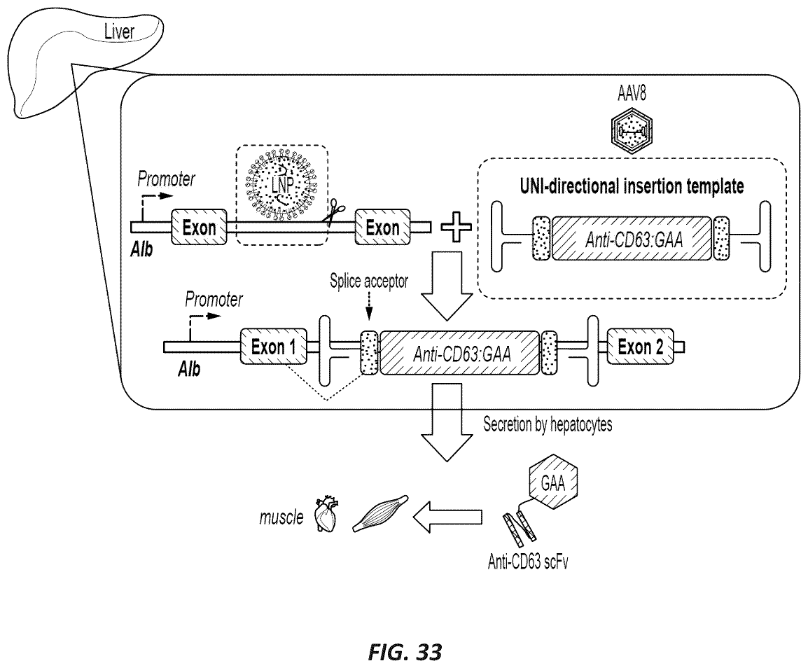

Exemplary components of the system for insertion for anti-CD63:GAA, including those used in subsequent examples, are described in more detail below. See FIGS. 31-33. The anti-CD63:GAA DNA template in the working examples described below is brought into the liver by a recombinant AAV8 vector, and the CRISPR/Cas9 RNA components (Cas9 mRNA and sgRNA) are delivered to the liver by LNP-mediated delivery (FIGS. 31 and 33). The anti-CD63:GAA protein produced by the liver is targeted to lysosomes in the muscle by targeting CD63, which is a rapidly internalizing protein highly expressed in the muscle. See FIG. 32. Single guide RNA, LNP-g9860, Cas9 mRNA, and LNP-g666 design and selection were as described in Example 1.

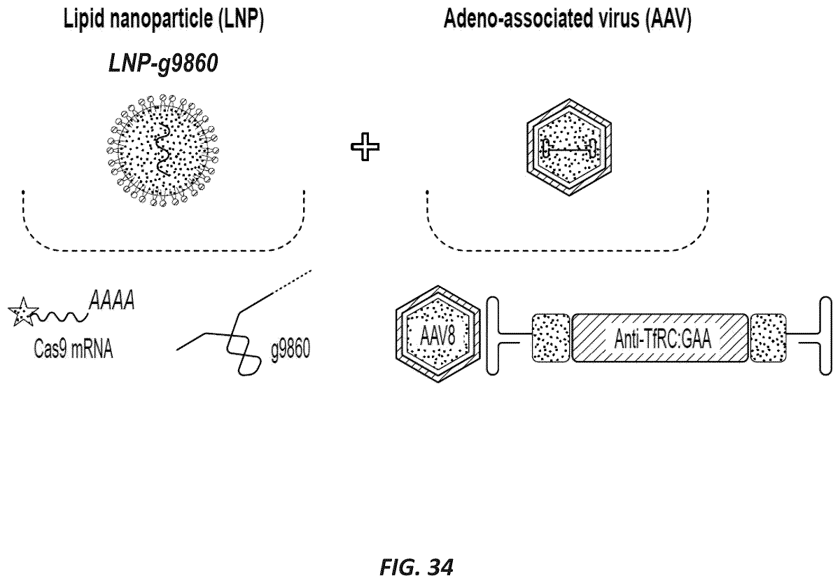

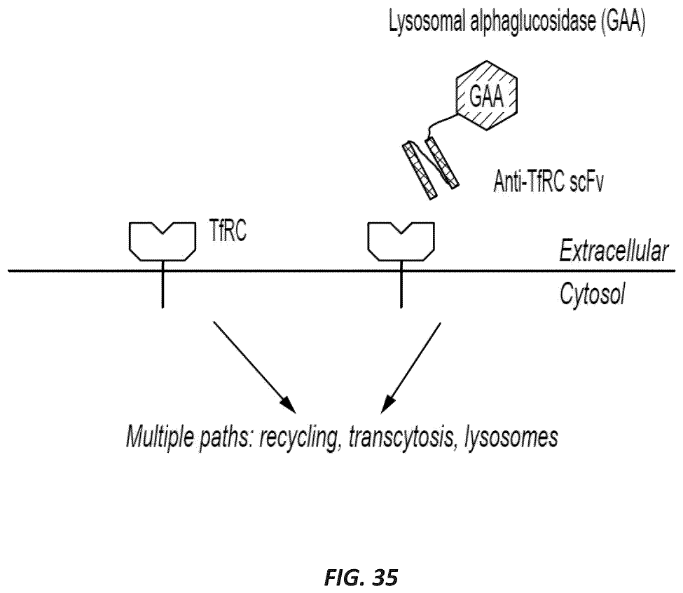

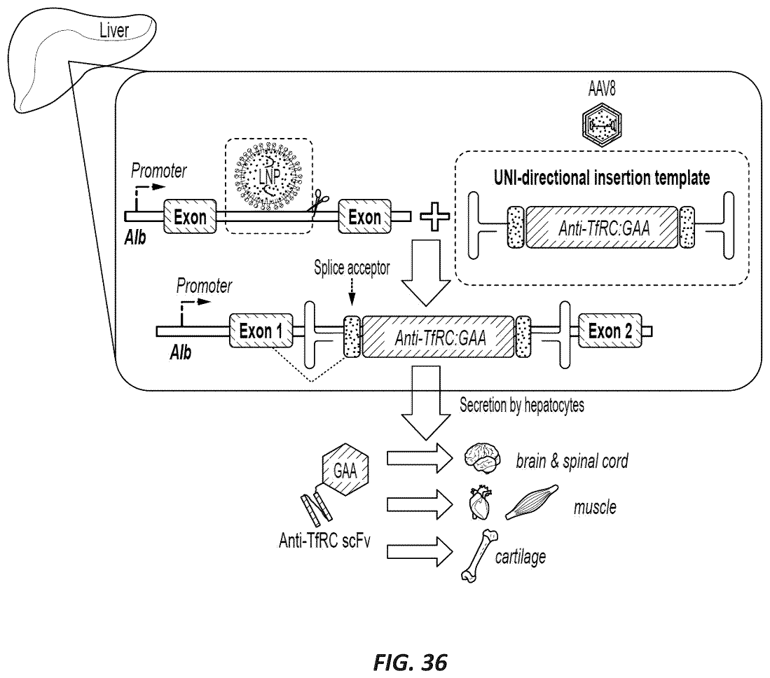

Exemplary components of the system for anti-TfR:GAA, including those used in subsequent examples, are described in more detail below. See FIGS. 34-36. The anti-TfR:GAA DNA templates in the working examples described below are brought into the liver by a recombinant AAV8 vector, and the CRISPR/Cas9 RNA components (Cas9 mRNA and sgRNA) are delivered to the liver by LNP-mediated delivery (FIGS. 34 and 36). The anti-TfR:GAA protein produced by the liver is targeted the muscle and CNS by targeting TfR, which is expressed in muscle and on brain endothelial cells. Transcytosis of TfR in these cells enables blood-brain-barrier crossing. See FIG. 35. Single guide RNA, LNP-g9860, Cas9 mRNA, and LNP-g666 design and selection were as described in Example 1.

### DNA Template Design and Selection

We engineered a DNA template for insertion of a nucleic encoding anti-CD63:GAA fusions in which the C-terminus of a single-chain fragment variable (scFv) is fused to the N-terminus of amino acids 70-952 of GAA with a glycine-serine linker. The GAA (70-952) sequence is set forth in SEQ ID NO: 727. The fusion protein encoded by the DNA template is set forth in SEQ ID NO: 733. A splice acceptor site is encoded upstream of the anti-CD63:GAA transgene, and a polyadenylation sequence is encoded downstream of the anti-CD63:GAA transgene. The splice acceptor sequence at the 5′ end of the transgene was derived from mouse Alb exon 2 splice acceptor. The polyadenylation sequence at the 3′ end of the transgene was derived from simian virus 40 (SV40).

We engineered DNA templates for insertion of a nucleic encoding anti-TfR:GAA fusions in which the C-terminus of a single-chain fragment variable (scFv) is fused to the N-terminus of amino acids 70-952 of GAA with a glycine-serine linker. The GAA (70-952) sequence is set forth in SEQ ID NO: 727. A splice acceptor site is encoded upstream of the anti-TfR:GAA transgene, and a polyadenylation sequence is encoded downstream of the anti-TfR:GAA transgene. The splice acceptor sequence at the 5′ end of the transgene was derived from mouse Alb exon 2 splice acceptor. The polyadenylation sequence at the 3′ end of the transgene was derived from simian virus 40 (SV40).

rAAV8 Vector

A recombinant AAV8 (rAAV8) vector was developed to carry the DNA insertion templates. The rAAV8 vector carrying the anti-CD63:GAA DNA template (REGV044) is a non-replicating vector that is an AAV-based vector derived from AAV serotype 8. The genome is a single-stranded deoxyribonucleic acid (DNA), comprising inverted terminal repeats (ITR) at each end. The ITRs flank the anti-CD63:GAA promoterless insertion template. The AAV ITRs flanking the cassette were derived from AAV2. The anti-CD63:GAA DNA template delivered by rAAV8 vector was designed as a promoterless template, thus relying on the targeted ALB locus promoter for expression.

The rAAV8 vector carrying the anti-TfR:GAA DNA template is a non-replicating vector that is an AAV-based vector derived from AAV serotype 8. The genome is a single-stranded deoxyribonucleic acid (DNA), comprising inverted terminal repeats (ITR) at each end. The ITRs flank the anti-TfR:GAA promoterless insertion template. The AAV ITRs flanking the cassette were derived from AAV2. The anti-TfR:GAA DNA template delivered by rAAV8 vector was designed as a promoterless template, thus relying on the targeted ALB locus promoter for expression.

### Example 3. Generation, Selection and Characterization of Immunoglobulin Molecules

Anti-human transferrin receptor (hTfR) antibodies were generated and screened for the ability to bind hTfR and for lack of strong blocking of human transferrin-hTfR binding.

Anti-hTfR Generation. VelocImmune mice were immunized with a recombinant protein comprising human transferrin receptor extracellular domain fused at N-terminus to a 6-His tag (referred to as human 6×His-TfR) as immunogen via subcutaneous footpad injection with Alum:CpG adjuvant. Mice bleeds were collected prior to the initial immunization injection and post-boost injections, and the immune sera were subjected to antibody titer determination using a human TfR specific enzyme-linked immunosorbent assay (ELISA). In this assay serum samples in serial dilutions were added to the immunogen coated plates and plate-bound mouse IgG were detected using an HRP-conjugated anti-mouse IgG antibody. Titer of a tested serum sample is defined as the extrapolated dilution factor of the sample that produces a binding signal two times of the signal of the buffer alone control sample. The mice with optimal anti-TfR antibody titers were selected and subjected to a final boost 3-5 days prior to euthanasia and splenocytes from these mice were harvested and subject to antibody isolation using B cell sorting technology (BST).

TfR specific antibodies of isolated antibodies were isolated and characterized. Two hundred and fourteen TfR-binding antibodies were cloned into single chain fragment variables (scFvs) in complementary orientations with either the variable heavy chain followed by the variable light chain (V_{H}-V_{K}), or the variable light chain followed by the variable heavy chain (V_{K}-V_{H}), and as fragment antigen-binding regions (Fabs). Conditioned media of CHO cell culture containing the scFvs or Fabs were tested for the ability to bind hTfR proteins and hTfR-expressing cells.

### Example 4. Binding Kinetics of 32 Anti-hTfR Primary Supernatants from CHO

Biacore binding kinetics assays were conducted for the interaction of 32 anti-human TfR IgG1 monoclonal antibodies from CHO supernatants with TfR reagents at 25° C.

TABLE 14
Monoclonal Antibody Clones Tested

| mAb# | AbID# | Source |
|---|---|---|
| 1 | 12795B | primary supernatant |
| 2 | 12798B | primary supernatant |
| 3 | 12799B | primary supernatant |
| 4 | 12801B | primary supernatant |
| 5 | 12802B | primary supernatant |
| 6 | 12808B | primary supernatant |
| 7 | 12812B | primary supernatant |
| 8 | 12834B | primary supernatant |
| 9 | 12835B | primary supernatant |
| 10 | 12839B | primary supernatant |
| 11 | 12841B | primary supernatant |
| 12 | 12843B | primary supernatant |
| 13 | 12844B | primary supernatant |
| 14 | 12845B | primary supernatant |
| 15 | 12847B | primary supernatant |
| 16 | 12848B | primary supernatant |
| 17 | 12850B | primary supernatant |
| 18 | 31863B | primary supernatant |
| 19 | 31874B | primary supernatant |
| 20 | 12816B | primary supernatant |
| 21 | 12833B | primary supernatant |
| 22 | 69261 | primary supernatant |
| 23 | 69263 | primary supernatant |
| 24 | 69305 | primary supernatant |
| 25 | 69307 | primary supernatant |
| 26 | 69323 | primary supernatant |
| 27 | 69326 | primary supernatant |
| 28 | 69329 | primary supernatant |
| 29 | 69331 | primary supernatant |
| 30 | 69332 | primary supernatant |
| 31 | 69340 | primary supernatant |
| 32 | 69348 | primary supernatant |
| 33 | REGN1945 | |

Reagents used:
REGN2431 (hmm.hTfRC; 79210 g/mol molecular weight), having the amino acid sequence:

(SEQ ID NO: 639)
HHHHHHEQKLISEEDLGGEQKLISEEDLCKGVEPKTECERLAGTESPVRE
EPGEDFPAARRLYWDDLKRKLSEKLDSTDFTGTIKLLNENSYVPREAGSQ
KDENLALYVENQFREFKLSKVWRDQHFVKIQVKDSAQNSVIIVDKNGRLV
YLVENPGGYVAYSKAATVTGKLVHANFGTKKDFEDLYTPVNGSIVIVRAG
KITFAEKVANAESLNAIGVLIYMDQTKFPIVNAELSFFGHAHLGTGDPYT
PGFPSFNHTQFPPSRSSGLPNIPVQTISRAAAEKLFGNMEGDCPSDWKTD
STCRMVTSESKNVKLTVSNVLKEIKILNIFGVIKGFVEPDHYVVVGAQRD
AWGPGAAKSGVGTALLLKLAQMFSDMVLKDGFQPSRSIIFASWSAGDFGS
VGATEWLEGYLSSLHLKAFTYINLDKAVLGTSNFKVSASPLLYTLIEKTM
QNVKHPVTGQFLYQDSNWASKVEKLTLDNAAFPFLAYSGIPAVSFCFCED
TDYPYLGTTMDTYKELIERIPELNKVARAAAEVAGQFVIKLTHDVELNLD
YERYNSQLLSFVRDLNQYRADIKEMGLSLQWLYSARGDFFRATSRLTTDF
GNAEKTDRFVMKKLNDRVMRVEYHFLSPYVSPKESPFRHVFWGSGSHTLP
ALLENLKLRKQNNGAFNETLFRNQLALATWTIQGAANALSGDVWDIDNE
F.

REGN2054 (mf TFRC ecto-mmh; 78500 g/mol molecular weight): monomeric monkey (cyno) Tfrc ectodomain (amino acids C89-F760,
   Accession #:XP_045243212.1) with a c-terminal myc-myc-hexahistidine tag containing a GG linker (underlined) between the 2 myc epitope sequences

(EQKLISEEDLGGEQKLISEEDLHHHHHH(SEQ ID NO: 640)).

Equilibrium dissociation constants (K_{D}) for the interaction of anti-TfR monoclonal antibodies with human and Fascicularis monkey TfR ecto domain recombinant proteins were determined using a real-time surface plasmon resonance (SPR) based Biacore S200 biosensor. All binding studies were performed in 10 mM HEPES, 150 mM NaCl, 3 mM EDTA, and 0.05% v/v surfactant Tween-20, pH 7.4 (HBS-EP) running buffer at 37° C. The Biacore CM5 sensor surface was first derivatized by amine coupling with a monoclonal mouse anti-human Fc antibody (REGN2567) followed by a step to capture anti-TfR monoclonal antibodies in CHO conditioned media. Human TfR extracellular domain expressed with a C-terminal myc-myc-hexahistidine tag (hTFR-mmh; REGN2431) or monkey TfR extracellular domain expressed with a C-terminal myc-myc-hexahistidine tag (mfTFR-mmh; REGN2054) at concentrations of 100 nM in HBS-EP running buffer were injected at a flow rate of 50 PL/min for 2 minutes. The dissociation of TfR bound to anti-TfR monoclonal antibodies was monitored for 3 minutes in HBS-EP running buffer. At the end of each cycle, the anti-TfR monoclonal antibodies capture surface was regenerated using a 12-sec injection of 20 mM H₃PO₄. The association rate (ka) and dissociation rate (kd) were determined by fitting the real-time binding sensorgrams to a 1:1 binding model with mass transport limitation using Scrubber 2.0c software. The dissociation equilibrium constant (K_{D}) and dissociative half-life (t½) were calculated from the kinetic rate constants as:

$${K}_{D} ( M ) = \frac{Kd}{ka} ,$$

$$and$$

$$t \frac{1}{2} ( min ) = \frac{ln ( 2 )}{60 * kd}$$

The equilibrium binding constant and the kinetic binding constants are summarized in Table 15 and Table 16 for human TfR and monkey TfR, respectively. At 25° C., anti-TfR monoclonal antibodies bound to hTfR-mmh with K_{D} values ranging from 65.6 μM to 41 nM, as shown in Table 15. Anti-TFR monoclonal antibodies bound to mfTFR-mmTh with K_{D} values ranging from 1.16 nM to 20.5 nM, as shown in Table 16.

Results are set forth below.

TABLE 15
Equilibrium and kinetic binding parameters for the interaction of hTFR-
mmh with anti-TfR monoclonal antibodies (bivalent IgG) at 25° C.

| | mAb | | | | | |
|---|---|---|---|---|---|---|
| | Capture | 100 nM Ag | kₐ | k_{d} | K_{D} | t½ |
| Molecule | Level (RU) | Bound (RU) | (1/Ms) | (1/s) | (M) | (min) |
| 12795B | 3344 | 883 | 1.22E+05 | <1.00E−05 | 8.23E−11 | >1155^{#} |
| 12798B | 4552 | 1151 | 1.63E+05 | 6.33E−05 | 3.87E−10 | 182.6 |
| 12799B | 2388 | 699 | 7.76E+04 | 6.62E−05 | 8.51E−10 | 174.5 |
| 12801B | 4720 | 598 | 8.19E+04 | 4.19E−05 | 5.13E−10 | 276.0 |
| 12802B | 2828 | 903 | 1.23E+05 | 2.92E−05 | 2.36E−10 | 395.4 |
| 12808B | 4336 | 964 | 1.19E+05 | 1.07E−04 | 8.94E−10 | 108.5 |
| 12812B | 2222 | 11 | 4.33E+05 | 1.31E−03 | 3.02E−09 | 8.8 |
| 12834B | 3837 | 11 | 6.00E+05 | 4.39E−03 | 7.00E−09 | 2.6 |
| 12835B | 3276 | 1146 | 1.34E+05 | 4.17E−04 | 3.11E−09 | 27.7 |
| 12839B | 5192 | 660 | 1.18E+05 | 5.33E−05 | 4.48E−10 | 216.9 |
| 12841B | 2895 | 1151 | 1.73E+05 | 1.16E−04 | 6.70E−10 | 99.8 |
| 12843B | 3080 | 854 | 9.42E+04 | 1.68E−04 | 1.78E−09 | 68.8 |
| 12844B | 2869 | 946 | 1.31E+05 | 1.22E−04 | 9.35E−10 | 95.0 |
| 12845B | 2911 | 1104 | 1.68E+05 | 1.99E−04 | 1.18E−09 | 57.9 |
| 12847B | 4425 | 895 | 1.05E+05 | 1.71E−04 | 1.62E−09 | 67.6 |
| 12848B | 4530 | 823 | 1.01E+05 | 4.99E−05 | 4.94E−10 | 231.6 |
| 12850B | 2990 | 725 | 8.37E+04 | 1.76E−05 | 2.15E−10 | 656.8 |
| 31863B | 5490 | 1083 | 1.52E+05 | <1.00E−05 | 6.56E−11 | >1155^{#} |
| 31874B | 5594 | 904 | 1.38E+05 | 1.15E−04 | 8.36E−10 | 100.5 |
| 12816B | 3377 | 357 | 5.79E+04 | 3.36E−04 | 5.80E−09 | 34.3 |
| 12833B | 3972 | 409 | 7.20E+04 | 2.52E−04 | 3.51E−09 | 45.9 |
| 69261 | 3546 | 462 | 7.00E+04 | 8.08E−05 | 1.16E−09 | 142.9 |
| 69263 | 180 | 150 | 1.77E+05 | 1.15E−03 | 6.50E−09 | 10.0 |
| 69305 | 2726 | 6 | NB* | NB* | NB* | NB* |
| 69307 | 2349 | 1285 | 1.90E+05 | 4.26E−05 | 2.26E−10 | 271.1 |
| 69323 | 4506 | 465 | 7.34E+04 | 1.80E−04 | 2.45E−09 | 64.1 |
| 69326 | 1709 | 680 | 1.15E+05 | 2.56E−04 | 2.22E−09 | 45.1 |
| 69329 | 1573 | 1100 | 1.92E+05 | 1.86E−04 | 9.70E−10 | 62.2 |
| 69331 | 4617 | 87 | 1.14E+05 | 4.66E−03 | 4.10E−08 | 2.5 |
| 69332 | 3915 | 9 | NB* | NB* | NB* | NB* |
| 69340 | 3226 | 999 | 1.44E+05 | 6.23E−05 | 4.29E−10 | 185.4 |
| 69348 | 2848 | 680 | 1.06E+05 | 1.72E−04 | 1.62E−09 | 67.2 |
| REGN1945 | 4011 | 6 | NB | NB | NB | NB |

^{#}indicates no dissociation was observed under the current experimental conditions and the kd value was manually fixed at 1.00E−05 s⁻¹ while fitting the real time binding sensorgrams.
*NB indicates that no binding was observed under the current experimental conditions.

TABLE 16
Equilibrium and kinetic binding parameters for the interaction of mfTfR-
mmh with anti-TfR monoclonal antibodies (bivalent IgG) at 25° C.

| | mAb | | | | | |
|---|---|---|---|---|---|---|
| | Capture | 100 nM Ag | kₐ | k_{d} | K_{D} | t½ |
| Molecule | Level (RU) | Bound (RU) | (1/Ms) | (1/s) | (M) | (min) |
| 12795B | 3334 | 364 | 6.10E+04 | 7.13E−05 | 1.16E−09 | 162.0 |
| 12798B | 4542 | 508 | 7.90E+04 | 1.29E−03 | 1.63E−08 | 9.0 |
| 12799B | 2384 | 651 | 8.64E+04 | 1.62E−04 | 1.88E−09 | 71.2 |
| 12801B | 4684 | 159 | 6.40E+04 | 6.53E−04 | 1.02E−08 | 17.7 |
| 12802B | 2819 | 464 | 7.05E+04 | 5.29E−04 | 7.51E−09 | 21.8 |
| 12808B | 4329 | 626 | 7.85E+04 | 4.54E−04 | 5.78E−09 | 25.5 |
| 12812B | 2220 | 1 | NB* | NB* | NB* | NB* |
| 12834B | 3820 | 5 | NB* | NB* | NB* | NB* |
| 12835B | 3262 | 254 | 9.51E+04 | 1.68E−03 | 1.77E−08 | 6.9 |
| 12839B | 5170 | 11 | NB* | NB* | NB* | NB* |
| 12841B | 2888 | 4 | NB* | NB* | NB* | NB* |
| 12843B | 3072 | 399 | 7.90E+04 | 1.18E−03 | 1.50E−08 | 9.8 |
| 12844B | 2857 | 437 | 7.59E+04 | 7.35E−04 | 9.68E−09 | 15.7 |
| 12845B | 2906 | 13 | NB* | NB* | NB* | NB* |
| 12847B | 4420 | 702 | 9.92E+04 | 3.33E−04 | 3.36E−09 | 34.7 |
| 12848B | 4524 | 261 | 5.29E+04 | 9.62E−04 | 1.82E−08 | 12.0 |
| 12850B | 2985 | 87 | 1.01E+05 | 1.57E−03 | 1.55E−08 | 7.3 |
| 31863B | 5480 | 145 | 1.63E+05 | 2.98E−03 | 1.83E−08 | 3.9 |
| 31874B | 5557 | 26 | 6.91E+05 | 9.96E−03 | 1.44E−08 | 1.2 |
| 12816B | 3376 | 315 | 6.52E+04 | 4.85E−04 | 7.44E−09 | 23.8 |
| 12833B | 3966 | 346 | 6.92E+04 | 4.86E−04 | 7.02E−09 | 23.8 |
| 69261 | 3537 | 331 | 6.83E+04 | 4.03E−04 | 5.90E−09 | 28.7 |
| 69263 | 181 | 77 | 1.97E+05 | 3.78E−03 | 1.92E−08 | 3.1 |
| 69305 | 2725 | −7 | NB* | NB* | NB* | NB* |
| 69307 | 2344 | 3 | NB* | NB* | NB* | NB* |
| 69323 | 4500 | 24 | 5.58E+05 | 1.06E−02 | 1.90E−08 | 1.1 |
| 69326 | 1707 | 9 | NB* | NB* | NB* | NB* |
| 69329 | 1571 | 8 | NB* | NB* | NB* | NB* |
| 69331 | 4611 | 26 | 4.89E+05 | 1.00E−02 | 2.05E−08 | 1.2 |
| 69332 | 3897 | 1 | NB* | NB* | NB* | NB* |
| 69340 | 3219 | 634 | 8.92E+04 | 7.23E−04 | 8.11E−09 | 16.0 |
| 69348 | 2851 | 433 | 9.60E+04 | 4.61E−04 | 4.80E−09 | 25.1 |
| REGN1945 | 4009 | 4 | NB | NB | NB | NB |

*NB indicates that no binding was observed under the current experimental conditions.

### Example 5. Anti-TfR Antibodies Blocking Human TfRC Monomer Binding to Human Holo-Transferrin by ELISA

An ELISA-based blocking assay was developed to determine the ability of anti-Transferrin Receptor (TfR) antibodies to block the binding of human Transferrin Receptor to human holo-transferrin ligand.

TABLE 17
Reagents

| Reagent | Source |
|---|---|
| Human Transferrin polyclonal goat IgG antibody | R&D Systems |
| Human Holo-Transferrin protein | R&D Systems |
| His-myc-myc-hTFRC ecto | Regeneron |
| HRP conjugated c-Myc polyclonal rabbit IgG antibody | Novus Biologicals |
| 1X PBS | Irvine Scientific |
| 1X PBST (0.05% tween-20 in PBS) | Sigma |
| BSA: albumin solution from bovine serum, 30% | Sigma |
| 3-3′,5-5′-tetramethylbenzidine (TMB) Substrate A | BD Biosciences |
| 3-3′,5-5′-tetramethylbenzidine (TMB) Substrate B | BD Biosciences |
| 2N Sulfuric Acid | VWR |
| Reacti-Bind 96-well plates corner notch | ThermoFisher Scientific |
| VWR 96-Well Deep Well Plates 0.5 ML | VWR |
| Aquamax Plate Washer 2000 | Molecular Devices |
| VICTOR ™ X4 Multilabel Plate Reader | PerkinElmer |

The human Transferrin Receptor recombinant protein, hTFRC, used in the experiment was comprised of hTfR extracellular domain (amino acids C89-F760) expressed with an N-terminal 6-Histidine-myc-myc tag (Hmm.hTfrc (REGN2431): Monomeric human Tfrc ectodomain (amino acids C89-F760, Accession #: NP_001121620.1) with an N-terminal hexahistidine-myc-myc-tag containing a GG linker (underlined) between the 2 myc epitope sequences (HHHHHHEQKLISEEDLGGEQKLISEEDL) (amino acids 1-28 of SEQ ID NO: 641)). The human holo-transferrin ligand protein (holo-Tf) isolated from human plasma was purchased from R&D Systems.

In the blocking assay, the anti-human Transferrin goat IgG polyclonal antibody (anti-hTf pAb) was passively absorbed at a concentration of 2 micrograms/mL in PBS on a 96-well microtiter plate overnight at 4° C. Nonspecific binding sites were subsequently blocked using a 0.5% (w/v) solution of BSA in PBS for 1 hour at room temperature. To the same plate, human holo-Tf was then added at a concentration of 1 micrograms/mL in PBS+0.5% BSA for 2 hours at room temperature. In a separate set of 96-well microtiter plates, solutions of 300 μM Hmm-hTFRC were mixed with TFRC antibody supernatants at 2-fold dilution. After a 1-hour incubation, the mixtures were transferred to the human holo-Tf microtiter plates. After another hour incubation at room temperature, plates were washed, and plate-bound Hmm-hTFRC was detected with horseradish peroxidase (HRP) conjugated rabbit anti-Myc polyclonal antibody. The plates were developed using TMB substrate solution according to the manufacturer's recommended procedure and absorbance at 450 nm was measured on a Victor™ Multilabel Plate Reader.

Percent blocking for the tested anti-TfR antibodies was calculated using the formula below:

$$\% Blocking = 100 - [ \frac{( Test antibody - Buffer alone without Hmm - hTFRC )}{( Hmm - hTFRC alone - Buffer alone without Hmm - hTFRC )} ] \times 100$$

Antibodies that blocked binding of Hmm-hTFRC to human holo-Tf equal or more than 50% were classified as blockers.

The ability of the anti-TfR antibody to block human TFRC binding to human holo-Tf was evaluated using an ELISA-based blocking assay. In this assay, a fixed concentration of Hmm-hTFRC was pre-incubated with anti-TfR antibody containing supernatant before binding to plate immobilized human holo-Tf protein, and the plate-bound Hmm-hTFRC was detected with HRP-conjugated c-Myc specific rabbit polyclonal antibodies.

Thirty-two anti-TfR antibodies cloned into single chain fragment variables (scFvs) in complementary orientations with either the variable heavy chain followed by the variable light chain (V_{H}-VK), or the variable light chain followed by the variable heavy chain (V_{K}-V_{H}) and also as fragment antigen-binding regions (Fabs). All ninety-six anti-TfR antibody supernatants were tested for the ability to block human TFRC binding to human holo-Tf. Ninety-four anti-TfR antibody supernatants showed no or low blocking activity with percentage blocking ranging from 0% to 45%, and these antibodies (Fabs or scFvs formats) were classified as non-blockers (Table 18). Only two Fab supernatants had blocking activity greater than 50%, with % blocking values of 64% and 78% respectively.

TABLE 18
Summary of Anti-TfR scFv and Fab Supernatants Ability to
Block Human TFRC binding to Immobilized Human Holo-Tf

| | Blocking of Hmm-hTFRC Binding to Human | | |
|---|---|---|---|
| | Holo-Tf, % Blocking | | |
| AbPID | Fab Format | scFv (V_{K}-V_{H}) Format | scFv (V_{H}-V_{K}) Format |
| 12795B | 44 | 23 | 45 |
| 12798B | 10 | 10 | 0 |
| 12799B | 5 | 10 | 26 |
| 12801B | 45 | 27 | 37 |
| 12802B | 15 | 17 | 11 |
| 12808B | 16 | 18 | 19 |
| 12812B | 14 | 12 | 19 |
| 12816B | 14 | 14 | 16 |
| 12833B | 64 | 40 | 22 |
| 12834B | −2 | 11 | −3 |
| 12835B | 78 | 37 | 45 |
| 12839B | 13 | 6 | 23 |
| 12841B | 29 | 1 | 10 |
| 12843B | 10 | 8 | 17 |
| 12844B | 20 | 10 | 12 |
| 12845B | 11 | 3 | 18 |
| 12847B | 3 | 13 | 11 |
| 12848B | 13 | 9 | 19 |
| 12850B | 18 | 8 | 10 |
| 31863B | 24 | 7 | 13 |
| 31874B | 16 | −1 | 14 |
| 69261 | 11 | 16 | 19 |
| 69263 | 14 | 4 | 14 |
| 69305 | 3 | 5 | 3 |
| 69307 | 12 | 12 | 9 |
| 69323 | 12 | 17 | 7 |
| 69326 | −2 | 12 | 18 |
| 69329 | 8 | 19 | 25 |
| 69331 | 9 | 13 | 7 |
| 69332 | 18 | 22 | 6 |
| 69340 | 3 | 13 | 6 |
| 69348 | 40 | 16 | 0 |

### Example 6. Anti-TFRC:GAA Gene Therapy

In this example, the ability of various anti-TFRC molecules to cross the blood-brain barrier and localize to the parenchyma of the brain was evaluated. Delivery of the molecules via episomal AAV liver depot was also evaluated along with rescue of the glycogen storage phenotype in various tissues.

### In Vivo Screening of Anti-hTFRC Scfv by HDD

To further evaluate the anti-human TFRC antibodies that were screened for binding in vitro, in vivo mouse studies in Tfrc^{hum/hum} knock-in mice were performed to evaluate blood-brain-barrier (BBB) crossing. This screen of 31 antibodies revealed 11 that had mature hGAA protein in brain homogenate detected by Western blot.

### GAA Fusions by Hydrodynamic Delivery (HDD)

Human TFRC knock-in mice were injected with DNA plasmids expressing the various anti-hTFRC antibodies in the anti-hTFRC scfv:2×G₄S(SEQ ID NO: 617):hGAA format under the liver-specific mouse TTR promoter. Mice received 50 μg of DNA in 0.9% sterile saline diluted to 10% of the mouse's body weight (0.1 mL/g body weight). Forty-eight hours post-injection, tissues were dissected from mice immediately after sacrifice by CO₂ asphyxiation, snap frozen in liquid nitrogen, and stored at −80° C.

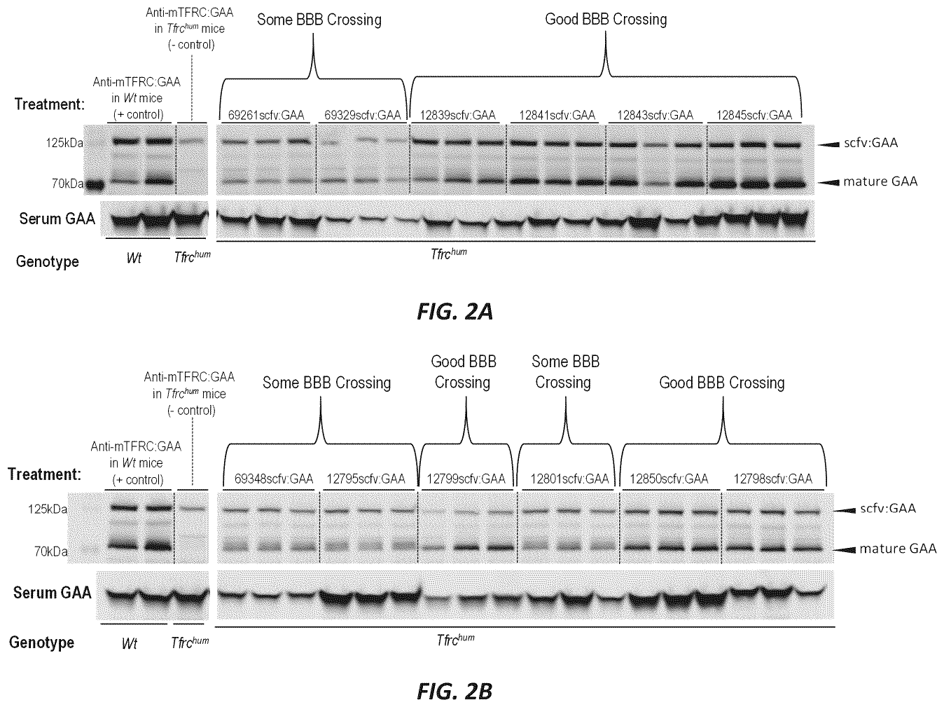

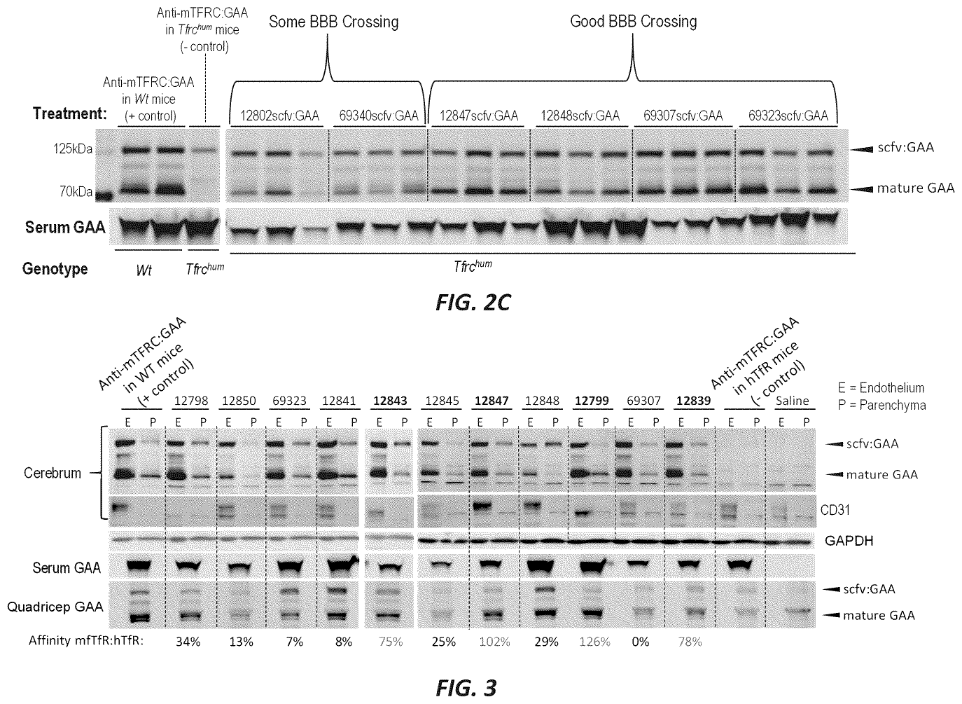

### Western Blot: (FIGS. 2A-2C)

Tissue lysates were prepared by lysis in RIPA buffer with protease inhibitors (1861282, Thermo Fisher, Waltham, MA, USA). Tissue lysates were homogenized with a bead homogenizer (FastPrep5, MP Biomedicals, Santa Ana, CA, USA). Cells or tissue lysates were run on SDS-PAGE gels using the Novex system (LifeTech Thermo, XPO4200BOX, LC2675, LC3675, LC2676). Gels were transferred to low-fluorescence polyvinylidene fluoridev (PVDF) membrane (IPFLO7810, LI-COR, Lincoln, NE, USA) and stained with Revert 700 Total Protein Stain (TPS; 926-11010 LI-COR, Lincoln, NE, USA), followed by blocking with Odyssey blocking buffer (927-500000, LI-COR, Lincoln, NE, USA) in Tris buffer saline with 0.1% Tween 20 and staining with antibodies against GAA (ab137068, Abcam, Cambridge, MA, USA), or anti-GAPDH (ab9484, Abcam, Cambridge, MA, USA) and the appropriate secondary (926-32213 or 925-68070, LI-COR, Lincoln, NE, USA). Blots were imaged with a LI-COR Odyssey CLx.

Protein band intensity was quantified in LI-COR Image Studio software. The quantification of the mature 77 kDa GAA band for each sample was determined by first normalizing to the lane's TPS signal, then normalizing to GAA levels in the serum (loading control and liver expression control, respectively). Values were then compared to the positive control group anti-mouse TFRC scfv:hGAA in Wt mice, and negative control group anti-mTFRC scfv:hGAA in Tfrc^{hum/hum} mice (FIGS. 2A-2C, Table 19).

TABLE 19
Quantification of mature hGAA protein in brain homogenate
from mice treated HDD with anti-hTFRC scfv:hGAA plasmids

| | | Mature hGAA protein in brain |
|---|---|---|
| Treatment group | Genotype | (normalized to positive control) |
| anti-mTFRCscfv:hGAA | Wt | 1.00 ± 0.43* |
| (positive control) | | |
| anti-mTFRCscfv:hGAA | Tfrc^{hum/hum} | 0.02 ± 0.03 |
| (negative control) | | |
| 69261scfv:hGAA | Tfrc^{hum/hum} | 0.67 ± 0.50 |
| 69307scfv:hGAA | Tfrc^{hum/hum} | 1.08 ± 0.19 |
| 69323scfv:hGAA | Tfrc^{hum/hum} | 0.91 ± 0.46 |
| 69329scfv:hGAA | Tfrc^{hum/hum} | 0.65 ± 0.13 |
| 69340scfv:hGAA | Tfrc^{hum/hum} | 0.55 ± 0.58 |
| 69348scfv:hGAA | Tfrc^{hum/hum} | 0.50 ± 0.05 |
| 12795scfv:hGAA | Tfrc^{hum/hum} | 0.27 ± 0.20 |
| 12798scfv:hGAA | Tfrc^{hum/hum} | 0.72 ± 0.42 |
| 12799scfv:hGAA | Tfrc^{hum/hum} | 1.05 ± 0.51* |
| 12801scfv:hGAA | Tfrc^{hum/hum} | 0.49 ± 0.18 |
| 12802scfv:hGAA | Tfrc^{hum/hum} | 0.29 ± 0.27 |
| 12839scfv:hGAA | Tfrc^{hum/hum} | 1.29 ± 0.27** |
| 12841scfv:hGAA | Tfrc^{hum/hum} | 1.72 ± 0.06*** |
| 12843scfv:hGAA | Tfrc^{hum/hum} | 1.79 ± 0.85*** |
| 12845scfv:hGAA | Tfrc^{hum/hum} | 3.08 ± 0.92*** |
| 12847scfv:hGAA | Tfrc^{hum/hum} | 1.24 ± 0.30 |
| 12848scfv:hGAA | Tfrc^{hum/hum} | 0.59 ± 0.16 |
| 12850scfv:hGAA | Tfrc^{hum/hum} | 0.47 ± 0.05 |

Data quantified from western blot as arbitrary units (FIGS. 2A-2C).
All values are mean ± SD, n = 3-6 per group.
One Way ANOVA vs. negative control anti-mTFRC scfv:hGAA in Tfrc^{hum/hum} mice;
*p < 0.05;
**p < 0.005;
***p < 0.0001

The control anti-mTRFC that was conjugated to GAA was 8D3 scFv. The 8D3 scFv has the heavy chain amino acid sequence:

(SEQ ID NO: 490)
EVQLVESGGGLVQPGNSLTLSCVASGFTFSNYGMHWIRQAPKKGLEWIAM
IYYDSSKMNYADTVKGRFTISRDNSKNTLYLEMNSLRSEDTAMYYCAVPT
SHYVVDVWGQGVSVTVSS,

and the light chain amino acid sequence:

(SEQ ID NO: 491)
DIQMTQSPASLSASLEEIVTITCQASQDIGNWLAWYQQKPGKSPQLLIYG
ATSLADGVPSRFSGSRSGTQFSLKISRVQVEDIGIYYCLQAYNTPWTFGG
GTKLELK.

Cap1llary Depletion of Brain Samples Following HDD of Anti-hTFRC Scfv:hGAA Plasmids

Anti-hTFRC scfv:hGAA molecules from Table 19 were tested in a secondary screen in Tfrc^{hum} mice to determine whether hGAA was present in the brain parenchyma, and not trapped in the BBB endothelial cells. Four molecules (12799, 12839, 12843, and 12847) identified in screen as being present in parenchyma based on mature hGAA in the parenchyma fraction on Western blot, as well as high affinity to cyno TFRC.

Animals were treated HDD as detailed above. Forty-eight hours post-injection, mice were perfused with 30 mL 0.9% saline immediately after sacrifice by CO₂ asphyxiation. A 2 mm coronal slice of cerebrum was taken between bregma and −2 mm bregma and placed in 700 uL physiological buffer (10 mM HEPES, 4 mM KCl, 2.8 mM CaCl₂, 1 mM MgSO₄, 1 mM NaH₂PO₄, 10 mM D-glucose in 0.9% saline pH 7.4) on ice. Brain slices were gently homogenized on ice with a glass dounce homogenizer. An equivalent volume of 26% dextran (MW 70,000 Da) in physiological buffer was added (final 13% dextran) and homogenized 10 more strokes. Parenchyma (supernatant) and endothelial (pellet) fractions were separated by centrifugation at 5,400g for 15 min at 4° C. Anti-hGAA western blot was performed on fractions as detailed above (FIG. 3, Table 20). Blots were also probed with anti-CD31 endothelial marker (Abcam ab182982).

TABLE 20
Quantification of mature hGAA protein in brain parenchyma fractions and BBB
endothelial fractions of mice treated HDD with anti-hTFRC scfv:hGAA plasmids

| | | Mature hGAA protein | Mature hGAA protein | Affinity to |
|---|---|---|---|---|
| | | in brain parenchyma | in brain endothelium | mfTFRC (% |
| | | (normalized to positive | (normalized to positive | of hTFRC |
| Treatment group | Genotype | control) | control) | binding) |
| anti- | Wt | 1.00 | 5.82 | ND |
| mTFRCscfv:hGAA | | | | |
| (positive control) | | | | |
| anti- | Tfrc^{hum/hum} | 0.00 | 0.01 | ND |
| mTFRCscfv:hGAA | | | | |
| (negative control) | | | | |
| 69307scfv:hGAA | Tfrc^{hum/hum} | 1.24 | 10.73 | 0% |
| 69323scfv:hGAA | Tfrc^{hum/hum} | 0.62 | 4.18 | 7% |
| 12798scfv:hGAA | Tfrc^{hum/hum} | 0.91 | 8.37 | 34% |
| 12799scfv:hGAA | Tfrc^{hum/hum} | 0.44 | 3.99 | 126% |
| 12839scfv:hGAA | Tfrc^{hum/hum} | 0.55 | 0.84 | 78% |
| 12841scfv:hGAA | Tfrc^{hum/hum} | 0.78 | 4.23 | 8% |
| 12843scfv:hGAA | Tfrc^{hum/hum} | 1.13 | 12.99 | 75% |
| 12845scfv:hGAA | Tfrc^{hum/hum} | 2.04 | 13.06 | 25% |
| 12847scfv:hGAA | Tfrc^{hum/hum} | 0.60 | 4.96 | 102% |
| 12848scfv:hGAA | Tfrc^{hum/hum} | 0.17 | 1.24 | 29% |
| 12850scfv:hGAA | Tfrc^{hum/hum} | 0.22 | 2.25 | 13% |

hGAA protein quantified from western blot as arbitrary units (FIG. 3).
n = 1 per group.
Affinity to cynomolgus macaque TFRC Luminex data, calculated as percent of binding to hTFRC: (mfTFRC binding ÷ hTFRC binding) × 100

TABLE 21
Quantification of hGAA protein in quadricep of mice
treated HDD with anti-hTFRC scfv:hGAA plasmids

| | | hGAA protein in quadricep |
|---|---|---|
| Treatment group | Genotype | (normalized to positive control) |
| Saline (vehicle) | Tfrc^{hum/hum} | 0.38 ± 0.25 |
| anti-mTFRCscfv:hGAA | Wt | 1.07 ± 0.27 |
| (positive control) | | |
| anti-mTFRCscfv:hGAA | Tfrc^{hum/hum} | 0.56 ± 0.17 |
| (negative control) | | |
| 69307scfv:hGAA | Tfrc^{hum/hum} | 0.58 ± 0.18 |
| 69323scfv:hGAA | Tfrc^{hum/hum} | 1.10 ± 0.19 |
| 12798scfv:hGAA | Tfrc^{hum/hum} | 1.33 ± 0.56 |
| 12799scfv:hGAA | Tfrc^{hum/hum} | 0.67 ± 0.18 |
| 12839scfv:hGAA | Tfrc^{hum/hum} | 1.80 ± 0.18 |
| 12841scfv:hGAA | Tfrc^{hum/hum} | 1.15 ± 0.12 |
| 12843scfv:hGAA | Tfrc^{hum/hum} | 1.78 ± 0.43 |
| 12845scfv:hGAA | Tfrc^{hum/hum} | 1.70 ± 1.33 |
| 12847scfv:hGAA | Tfrc^{hum/hum} | 7.74 ± 9.42 |
| 12848scfv:hGAA | Tfrc^{hum/hum} | 0.82 ± 0.18 |
| 12850scfv:hGAA | Tfrc^{hum/hum} | 0.76 ± 0.34 |

Cap1llary Depletion of Mouse Brain Samples Following Liver-Depot AAV8 Anti-hTFRC Scfv:hGAA Treatment

To confirm our HDD screen findings in a more long-term treatment model, we treated Tfrc^{hum} mice with anti-hTFRC scfv:hGAA_molecules delivered as episomal liver depot AAV8 anti-hTFRC scfv:GAA under the TTR promoter. We found that all 4 molecules (12799, 12843, 12847 and 12839) delivered mature hGAA to the brain parenchyma when delivered as AAV8.

### AAV Production and In Vivo Transduction

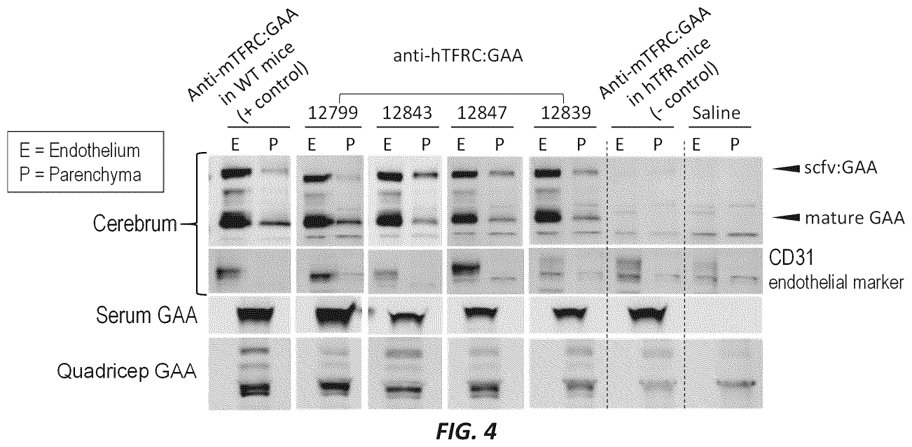

Recombinant AAV8 (AAV2/8) was produced in HEK293 cells. Cells were transfected with three plasmids encoding adenovirus helper genes, AAV8 rep and cap genes, and recombinant AAV genomes containing transgenes flanked by AAV2 inverted terminal repeats (ITRs). On day 5, cells and medium were collected, centrifuged, and processed for AAV purification. Cell pellets were lysed by freeze-thaw and cleared by centrifugation. Processed cell lysates and medium were overlaid onto iodixanol gradients columns and centrifuged in an ultracentrifuge. Virus fractions were removed from the interface between the 40% and 60% iodixanol solutions and exchanged into 1×PBS with desalting columns. AAV vg (vg=viral genomes) were quantified by ddPCR. AAVs were diluted in PBS+0.001% F-68 Pluronic immediately prior to injection. Tfrc^{hum} mice were dosed with 3e12 vg/kg body weight in a volume of ˜100 uL. Mice were sacrificed 4 weeks post injection and capillary depletion and western blotting were performed as described above (FIG. 4, Table 22).

TABLE 22
Quantification of mature hGAA protein in brain parenchyma fractions and BBB endothelial
fractions of mice treated with liver-depot AAV8 anti-hTFRC scfv:hGAA

| | | Mature hGAA protein in brain | Mature hGAA protein in brain |
|---|---|---|---|
| | | parenchyma (normalized to | endothelium (normalized to |
| Treatment group | Genotype | positive control) | positive control) |
| anti- | Wt | 1.00 | 1.00 |
| mTFRCscfv:hGAA | | | |
| (positive control) | | | |
| anti- | Tfrc^{hum/hum} | 0.02 | 0.01 |
| mTFRCscfv:hGAA | | | |
| (negative control) | | | |
| 12799scfv:hGAA | Tfrc^{hum/hum} | 0.94 | 0.94 |
| 12839scfv:hGAA | Tfrc^{hum/hum} | 0.49 | 0.62 |
| 12843scfv:hGAA | Tfrc^{hum/hum} | 0.61 | 0.63 |
| 12847scfv:hGAA | Tfrc^{hum/hum} | 1.90 | 1.33 |

Data quantified from western blot as arbitrary units (FIG. 4).
n = 1 per group

Rescue of Glycogen Storage Phenotype in Gaa^{-/-}/Tfrc^{hum} Mice with AAV8 Episomal Liver Depot Anti-hTFRC Scfv:GAA

Anti-hTFRC scfv:GAA molecules in Pompe disease model mice were tested to determine whether anti-hTFRCscfv:GAA rescued the glycogen storage phenotype. The molecules, 12839, 12843, 12847, normalized glycogen to Wt levels. (12799 not tested).

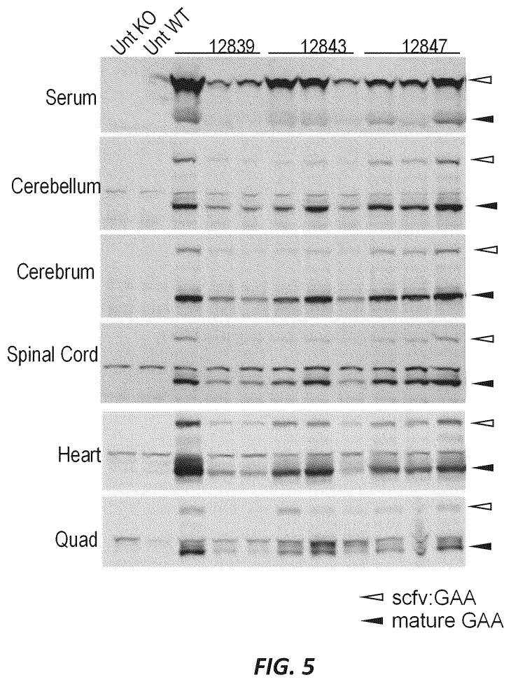

AAV production and in vivo transduction were performed as above. Gaa^{-/-}/Tfrc^{hum}mice were dosed with 2e12 vg/kg AAV8. Tissues were harvested 4 weeks post-injection and flash-frozen as above. hGAA Western blot was performed as above (FIG. 5, Table 23).

### Glycogen Quantification

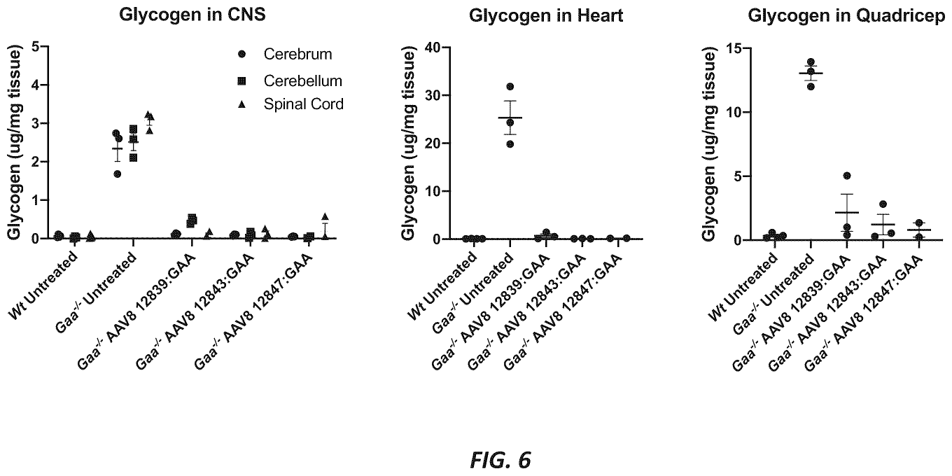

Tissues were dissected from mice immediately after sacrifice by CO₂ asphyxiation, snap frozen in liquid nitrogen, and stored at −80° C. Tissues were lysed on a benchtop homogenizer with stainless steel beads in distilled water for glycogen measurements or RIPA buffer for protein analyses. Glycogen analysis lysates were boiled and centrifuged to clear debris. Glycogen measurements were performed fluorometrically with a commercial kit according to manufacturer's instructions (K646, BioVision, Milpitas, CA, USA). See Table 24 and FIG. 6.

TABLE 23
Quantification of hGAA protein in tissues of Gaa^{-/-}/Tfrc^{hum} mice treated with liver-depot AAV8 anti-hTFRC scfv:hGAA

| Treatment group | n | Serum * | Liver * | Cerebrum ** | Cerebellum ** | Spinal Cord ** | Heart ** | Quadricep ** |
|---|---|---|---|---|---|---|---|---|
| Gaa^{-/-} Untreated | 1 | 0.00 | 0.02 | 0.00 | 0.00 | 0.00 | 0.02 | 0.01 |
| Gaa^{-/-} 12839scfv:hGAA | 3 | 2.42 ± 2.41 | 1.63 ± 0.96 | 0.14 ± 0.12 | 0.13 ± 0.12 | 0.19 ± 0.19 | 0.53 ± 0.52 | 0.14 ± 0.16 |
| Gaa^{-/-} 12843scfv:hGAA | 3 | 2.07 ± 1.35 | 2.23 ± 0.08 | 0.17 ± 0.07 | 0.11 ± 0.05 | 0.17 ± 0.09 | 0.49 ± 0.31 | 0.18 ± 0.06 |
| Gaa^{-/-} 12847scfv:hGAA | 3 | 1.56 ± 0.71 | 1.40 ± 0.13 | 0.25 ± 0.04 | 0.21 ± 0.09 | 0.42 ± 0.19 | 0.58 ± 0.17 | 0.19 ± 0.08 |

Data quantified from western blot as arbitrary units (FIG. 5).
All values are mean ± SD, n = 1-3 per group.
* Total hGAA protein;
** Mature hGAA protein

TABLE 24
Quantification of glycogen in tissues of Gaa^{-/-}/
Tfrc^{hum} mice treated with liver-depot AAV8 anti-hTFRC scfv:hGAA

| Treatment group | Cerebrum | Cerebellum | Spinal Cord | Heart | Quadricep |
|---|---|---|---|---|---|
| Wt Untreated | 0.06 ± 0.04* | 0.01 ± 0.04* | 0.05 ± 0.05* | 0.08 ± 0.02* | 0.34 ± 0.19* |
| Gaa−/− Untreated | 2.34 ± 0.58 | 2.51 ± 0.38 | 3.08 ± 0.23 | 25.30 ± 6.06 | 13.05 ± 0.98 |
| Gaa−/− 12839scfv:hGAA | 0.11 ± 0.03* | 0.46 ± 0.08* | 0.08 ± 0.10* | 0.68 ± 0.68* | 2.15 ± 2.52* |
| Gaa−/− 12843scfv:hGAA | 0.09 ± 0.02* | 0.09 ± 0.08* | 0.13 ± 0.13* | 0.09 ± 0.01* | 1.22 ± 1.39* |
| Gaa−/− 12847scfv:hGAA | 0.05 ± 0.01* | 0.02 ± 0.03* | 0.20 ± 0.33* | 0.11 ± 0.11* | 0.80 ± 0.79* |

All values are glycogen ug/mg tissue, mean ± SD, n = 3-4 per group.
One Way ANOVA
*p < 0.0001 vs. Gaa^{-/-} Untreated group

Rescue of Glycogen Storage in Brain and Muscle in Gaa^{-/-}/Tfrc^{hum} Mice with AAV8 Episomal Liver Depot Anti-hTFRC Scfv:GAA

Anti-hTFRCscfv:GAA molecules, 12799, 12843, and 12847, were tested in Pompe disease model mice to determine whether they rescued the glycogen storage phenotype. Histology was performed on brain and muscle sections to visualize glycogen in the tissues. All 3 molecules reduced glycogen staining in the brain and muscle.

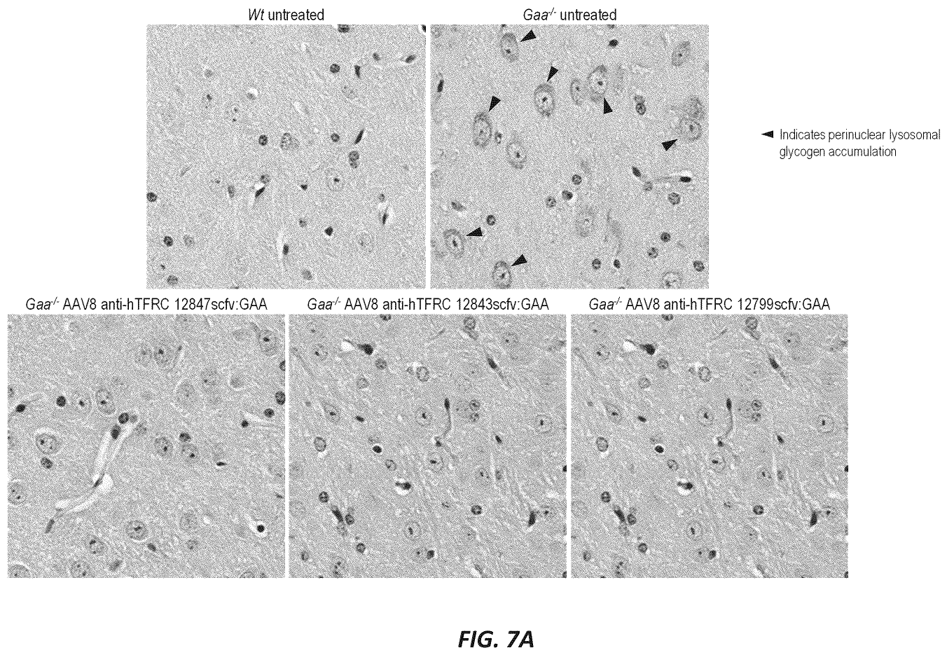

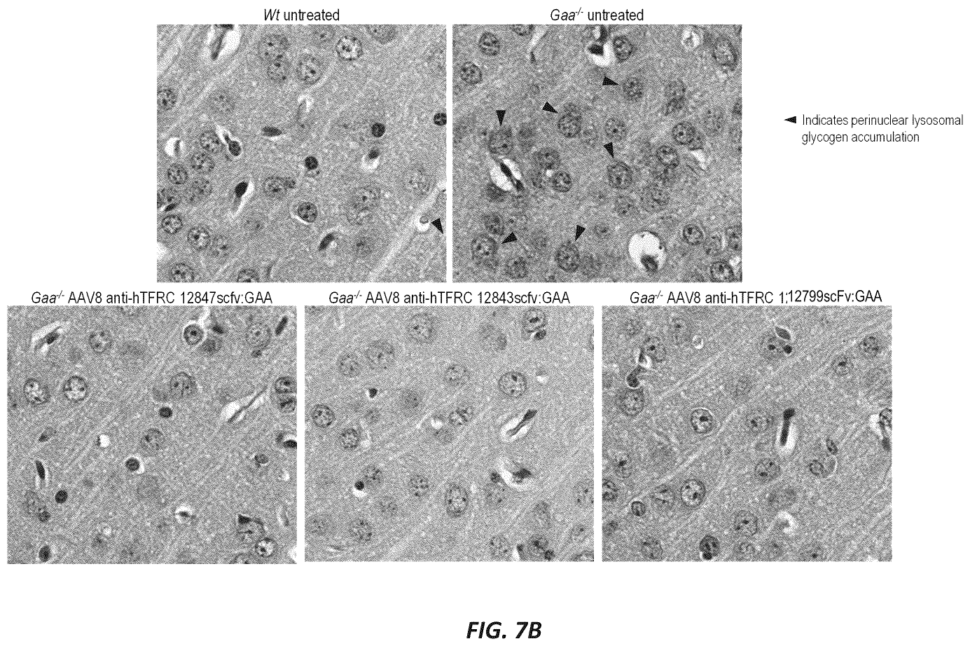

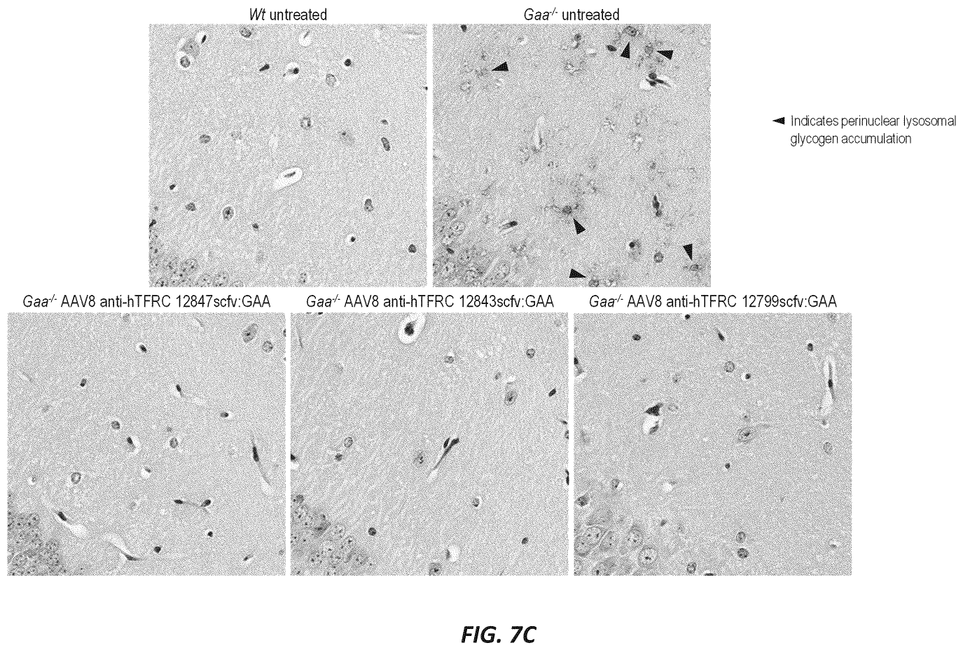

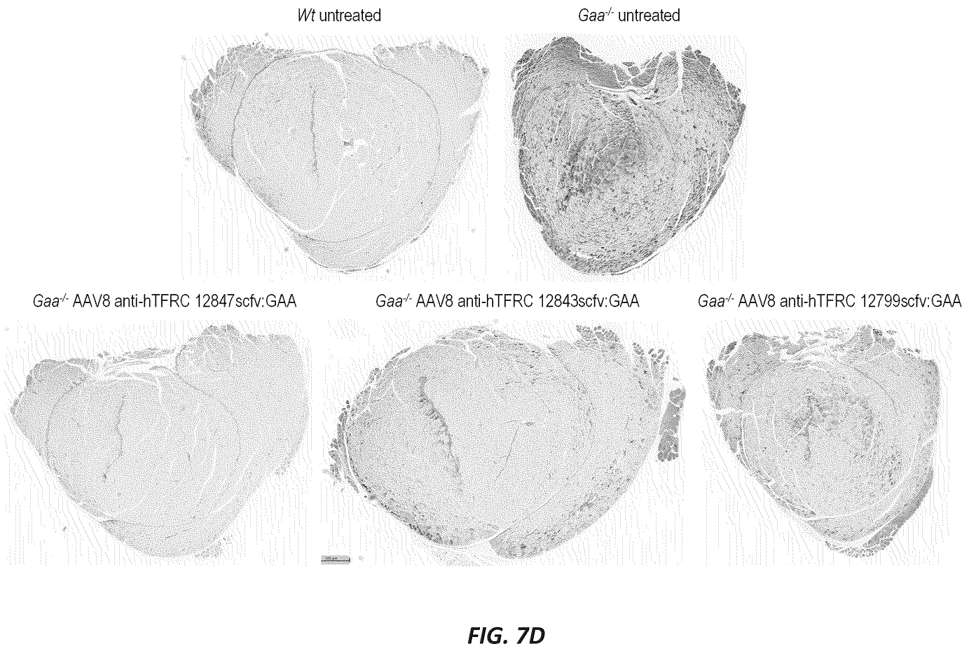

AAV production and in vivo transduction were performed as above. Three month old Gaa^{-/-}/Tfrc^{hum} mice were dosed with 4e11 vg/kg AAV8. Four weeks post-injection, tissues were frozen for glycogen analysis as above (Table 25). For histology, animals were perfused with saline (0.9% NaCl), and tissues were drop-fixed overnight in 10% Normal Buffered Formalin. Tissues were washed 3× in PBS and stored in PBS/0.01% sodium azide until embedding. Tissues were embedded in paraffin and 5 um sections were cut from brain (coronal, −2 mm bregma) and quadricep (fiber cross-section). Sections were stained with Periodic Acid-Schiff and Hematoxylin using standard protocols (FIGS. 7A-7D).

TABLE 25
Quantification of glycogen in tissues of Gaa^{-/-}/
Tfrc^{hum} mice treated with liver-depot AAV8 anti-hTFRC scfv:hGAA

| Treatment group | Cerebellum | Quadricep |
|---|---|---|
| Wt Untreated | 0.02 ± 0.03* | 0.55 ± 0.10* |
| Gaa−/− Untreated | 1.91 ± 0.26 | 12.19 ± 3.02 |
| Gaa−/− 12799scfv:hGAA | 0.10 ± 0.06* | 1.34 ± 0.9* |
| Gaa−/− 12843scfv:hGAA | 0.09 ± 0.06* | 1.09 ± 1.27* |
| Gaa−/− 12847scfv:hGAA | 0.07 ± 0.06* | 0.72 ± 0.64* |

All values are glycogen ug/mg tissue, mean ± SD, n = 5-8 per group.
One Way ANOVA
*p < 0.0001 vs. Gaa^{-/-} Untreated group

### Example 7. Iron Assay

This Example evaluated the effect of anti-TfR antigen-binding proteins on iron homeostasis in mice.

### Validating TFRC Expression in Tfrc^{hum} Mice and Assessing Iron Homeostasis

To validate that Tfrc^{hum} mice expressed TFRC at physiological levels and had normal iron homeostasis, we compared Tfrc^{hum} mice t0 Wt mice and quantified expression of TFRC in tissues, serum markers, tissue iron content, and transferrin in tissues. Overall, TFRC expression and iron homeostasis was normal in the Tfrc^{hum} mice.

Six month old Wt mice (11 males, 4 females) and Tfrc^{hum} mice (10 males, 8 females) were analyzed in this experiment. Tissues were dissected from mice immediately after sacrifice by CO₂ asphyxiation, snap frozen in liquid nitrogen, and stored at −80° C.

Tfrc RNA Quantification by qPCR

Total RNA was isolated from tissues with Trizol following manufacturer protocol (ThermoFisher 15596026). Tfrc RNA was quantified by Tagman qPCR (ThermoFisher) following standard protocols using universal primers to exon 1 that amplify from both Wt and Tfrc^{hum} mice (GCTGCATTGCGGACTGTAGA (SEQ ID NO: 642)/TCCATCATTCTCAGCTGCTACAA (SEQ ID NO: 643)). ΔΔCT values were calculated relative to the Wt male group. Data in Table 26.

### Serum Assays

Blood was collected from mice by cardiac puncture immediately following CO₂ asphyxiation and serum was separated using serum separator tubes (BD Biosciences, 365967). Serum iron and Total Iron Binding Content (TIBC) were quantified using standard protocols. Serum hepcidin was quantified by ELISA kit (Intrinsic Life Sciences SKU HMC-001). Data in Table 27.

### Tissue Iron Content

Wet tissue was weighed to achieve uniformity and then dried for 72 hours in an open tube at 56° C. Tissue was then placed in digestion buffer (10% Tricloroacetic acid and 37% HCL) and heated at 65° C. for 48 hours. To assay iron content, the supernatant was placed in a 96 well plate and incubated in a color development solution (Thioglycolic acid, bathophenanthroline acid and sodium acetate). Absorbance was read on a Spectramax i3 by Molecular Devices and Graph Pad Prism was used to interpolate the sample absorbance values read against a standard curve to calculate iron content in the whole piece of tissue. Iron content was then calculated based on dry weight. Data in Table 28.

### Transferrin ELISA

All tissues were homogenized using a Fastprep-24 5G from MP Biomedicals. Prior to homogenization, tissues were placed in RIPA buffer with phosphatase and HALT protease inhibitors (ThermoFisher), homogenized with their organ specific protocol and then centrifuged to pellet debris. The supernatant was collected and assayed for total protein using a Pierce BCA Protein Assay Kit. Absorbance was measured on a Spectramax i3 by Molecular Devices. Once total protein was measured, all samples were diluted to match the least concentrated sample so loading would be uniform for the ELISA. Kits obtained from Abcam were used to measure the presence of total transferrin in tissue homogenate (Abcam ab157724). Plates were run in accordance with the supplied protocol using the provided reagents and absorbance was read on a Spectramax i3 by Molecular Devices. Graph Pad Prism was used to interpolate the sample absorbance values read against a standard curve. Data in Table 29.

TABLE 26
Tfrc RNA quantification in untreated Wt and Tfrc^{hum} mice

| Genotype | Sex | Liver Tfrc | Quadricep Tfrc | Brain Tfrc |
|---|---|---|---|---|
| Wt | M | 1.02 ± 0.21 | 1.10 ± 0.53 | 1.02 ± 0.21 |
| Wt | F | 1.11 ± 0.64 | 0.60 ± 0.17 | 1.03 ± 0.13 |
| Tfrc^{hum} | M | 1.14 ± 0.28 | 1.02 ± 0.39 | 1.86 ± 0.35* |
| Tfrc^{hum} | F | 0.75 ± 0.22 | 0.43 ± 0.93 | 1.88 ± 0.25* |

All values are ΔΔCT vs. Wt males group, mean ± SD, n = 4-11 per group.
One Way ANOVA
*p < 0.001 vs. Wt sex-matched group

TABLE 27
Serum iron markers in untread Wt and Tfrc^{hum} mice

| Genotype | Sex | Serum Iron ug/dL | Serum TIBC ug/dL | Serum Hepcidin ng/mL |
|---|---|---|---|---|
| Wt | M | 146.73 ± 20.30 | 360.18 ± 27.02 | 416.73 ± 133.04 |
| Wt | F | 125.50 ± 9.04 | 342.25 ± 22.25 | 436.35 ± 143.28 |
| Tfrc^{hum} | M | 173.00 ± 12.77* | 351.20 ± 21.94 | 415.86 ± 101.27 |
| Tfrc^{hum} | F | 156.75 ± 14.18* | 353.50 ± 17.03 | 523.30 ± 175.70 |

All values are mean ± SD, n = 4-11 per group.
All values are within normal physiological range.
One Way ANOVA
*p < 0.05 vs. Wt sex-matched group

TABLE 28
Tissue iron quantification in untread Wt and Tfrc^{hum} mice

| Genotype | Sex | Liver | Spleen |
|---|---|---|---|
| Wt | M | 307.03 ± 32.74 | 1666.38 ± 239.18 |
| Wt | F | 507.45 ± 110.45 | 1833.12 ± 173.36 |
| Tfrc^{hum} | M | 300.00 ± 33.77 | 1818.44 ± 276.86 |
| Tfrc^{hum} | F | 638.46 ± 139.03 | 1695.96 ± 140.02 |

All values are ug/g dry tissue, mean ± SD, n = 4-11 per group.
Values are non-significant (One Way ANOVA vs. Wt sex-matched group).

TABLE 29
Transferrin protein in untreated Wt and Tfrc^{hum} mice (ELISA)

| Genotype | Sex | Serum | Liver | Cerebrum |
|---|---|---|---|---|
| Wt | M | 1191.28 ± 137.03 | 32.61 ± 9.87 | 7.35 ± 1.30 |
| Wt | F | 1270.81 ± 138.42 | 27.01 ± 13.22 | 6.33 ± 0.93 |
| Tfrc^{hum} | M | 1251.40 ± 113.59 | 32.97 ± 7.26 | 7.92 ± 1.63 |
| Tfrc^{hum} | F | 1425.89 ± 290.77 | 40.17 ± 8.22 | 8.26 ± 2.08 |

All values are ug/mL homogenate normalized to protein content; mean ± SD, n = 4-11 per group.
Values are non-significant (One Way ANOVA vs. Wt sex-matched group).

Rescue of glycogen storage in brain and muscle in Gaa^{-/-}/Tfrc^{hum} mice with AAV8 episomal liver depot anti-hTFRC scfv:GAA

We tested the anti-hTFRC scfv:GAA leads 12799, 12843, and 12847 in Pompe disease model mice to determine whether anti-hTFRC scfv:GAA rescued the glycogen storage phenotype (glycogen data in other data package). Here we also tested whether treatment with anti-TFRCscfv:GAA leads altered iron homeostasis (Tables 30, 31, and 32). We found that 4-week treatment did not affect iron homeostasis with any of the leads.

TABLE 30
Serum iron markers in Gaa^{-/-}/Tfrc^{hum} mice treated
with AAV8 episomal liver depot anti-hTFRC scfv:GAA

| | Serum iron | Serum TIBC | Serum Hepcidin |
|---|---|---|---|
| Treatment group | ug/dL | ug/dL | ng/mL |
| Wt Untreated | 203.83 ± 29.49 | 334.33 ± 17.83 | 265.89 ± 60.71 |
| Gaa^{-/-} | 196.50 ± 25.15 | 326.50 ± 34.39 | 329.19 ± 124.11 |
| Untreated | | | |
| Gaa^{-/-} | 188.50 ± 32.83 | 319.14 ± 28.20 | 341.25 ± 104.87 |
| 12799scfv:hGAA | | | |
| Gaa^{-/-} | 163.63 ± 28.27 | 275.88 ± 65.67 | 298.47 ± 104.60 |
| 12843scfv:hGAA | | | |
| Gaa^{-/-} | 159.29 ± 19.09 | 323.00 ± 24.82 | 387.47 ± 69.56 |
| 12847scfv:hGAA | | | |

All values are mean ± SD, n = 5-8 per group.
All values are non-significant (One Way ANOVA)

TABLE 31
Tissue iron quantification in Gaa^{-/-}/Tfrc^{hum} mice treated
with AAV8 episomal liver depot anti-hTFRC scfv:GAA

| Treatment group | Liver | Heart | Spleen |
|---|---|---|---|
| Wt Untreated | 228.12 ± 37.65 | 349.78 ± 27.98 | 893.68 ± 216.93 |
| Gaa^{-/-} | 260.59 ± 49.54 | 355.82 ± 48.43 | 1258.57 ± 600.35 |
| Untreated | | | |
| Gaa^{-/-} | 285.07 ± 67.17 | 350.44 ± 51.70 | 1251.36 ± 628.45 |
| 12799scfv:hGAA | | | |
| Gaa^{-/-} | 279.64 ± 41.89 | 360.78 ± 37.34 | 906.81 ± 280.82 |
| 12843scfv:hGAA | | | |
| Gaa^{-/-} | 336.33 ± 85.74 | 391.67 ± 58.36 | 1773.74 ± 374.26 |
| 12847scfv:hGAA | | | |

All values are ug/g dry tissue, mean ± SD, n = 5-8 per group.
All values are non-significant (One Way ANOVA).

TABLE 32
Transferrin protein in Gaa^{-/-}/Tfrc^{hum} mice treated with
AAV8 episomal liver depot anti-hTFRC scfv:GAA (ELISA)

| Treatment group | Liver | Spleen | Cerebrum |
|---|---|---|---|
| Wt Untreated | 19.82 ± 4.73 | 3.17 ± 1.46 | 10.69 ± 1.05 |
| Gaa^{-/-} Untreated | 14.71 ± 7.37 | 6.36 ± 2.59 | 12.54 ± 2.07 |
| Gaa^{-/-} 12799scfv:hGAA | 16.66 ± 6.99 | 5.87 ± 2.48 | 10.34 ± 1.49 |
| Gaa^{-/-} 12843scfv:hGAA | 14.16 ± 5.93 | 5.67 ± 1.95 | 11.19 ± 2.56 |
| Gaa^{-/-} 12847scfv:hGAA | 13.81 ± 3.04 | 5.70 ± 1.30 | 13.72 ± 1.87 |

All values are ug/mL homogenate normalized to protein content; mean ± SD, n = 5-8 per group.
Values are non-significant (One Way ANOVA vs. Gaa^{-/-} Untreated group).

### Example 8. Insertion Anti-hTFRC:GAA Gene Therapy in Mice

### mAb Clone IDs

H1H12847B in scfv:GAA format (REGN16826)
12450NVH in scfv:GAA format (comparator, REGN5534)
   Insertion of Anti-hTFRC 12847Scfv:GAA in Gaa^{-/-}/Tfrch^{hum/hum} Mice

We tested our lead anti-hTFRC 12847scfv:GAA in Pompe disease model mice by albumin insertion to determine whether we could replicate the results we saw with episomal AAV8 liver depot expression. Albumin insertion of 12847scfv:GAA delivered mature hGAA protein to the brain and muscle, and rescued the glycogen storage phenotype in Gaa^{-/-}/Tfrc^{hum/hum} mice. These data were produced with the native 12847scfv:GAA sequence that is not optimized.

We compared 12847scfv:GAA to the muscle-targeted anti-hCD63scfv:GAA in Gaa^{-/-}/Cd63^{hum} mice.

### AAV Production

A promoterless AAV genome plasmid was created with the 12847scfv:GAA sequence and the mouse albumin exon 1 splice acceptor site at the 3′ end. Recombinant AAV8 (AAV2/8) was produced in HEK293 cells. Cells were transfected with three plasmids encoding adenovirus helper genes, AAV8 rep and cap genes, and recombinant AAV genomes containing transgenes flanked by AAV2 inverted terminal repeats (ITRs). On day 5, cells and medium were collected, centrifuged, and processed for AAV purification. Cell pellets were lysed by freeze-thaw and cleared by centrifugation. Processed cell lysates and medium were overlaid onto iodixanol gradients columns and centrifuged in an ultracentrifuge. Virus fractions were removed from the interface between the 40% and 60% iodixanol solutions and exchanged into 1×PBS with desalting columns. AAV vg were quantified by ddPCR.

In Vivo CRISPR/Cas9 Insertion into the Albumin Locus

Three month old Gaa^{-/-}/Tfrc^{hum/hum} mice were dosed via tail vein injection with 3e12 vg/kg AAV8 12847scfv:GAA and 3 mg/kg LNP gRNA/Cas9 mRNA diluted in PBS+0.001% F-68 Pluronic. Mice were sacrificed 3 weeks post injection. Negative control mice received insertion AAV8 without LNP. Positive control mice were dosed with 4e11 vg/kg episomal liver depot AAV8 12847scfv:GAA under the TTR promoter (phenotype rescue data previously shown). Tissues were dissected from mice immediately after sacrifice by CO₂ asphyxiation, snap frozen in liquid nitrogen, and stored at −80° C. Blood was collected from mice by cardiac puncture immediately following CO₂ asphyxiation and serum was separated using serum separator tubes (BD Biosciences, 365967).

TABLE 33
Treatment groups and controls

| | Geno- | |
|---|---|---|
| Treatment group | type | Function |
| Wt Untreated | Tfrc^{hum} | Normal untreated mouse control |
| Gaa^{-/-} untreated | Gaa^{-/-}/ | Untreated Pompe disease mouse |
| | Tfrc^{hum} | |
| Gaa^{-/-} insertion | Gaa^{-/-}/ | Negative control for insertion |
| AAV only | Tfrc^{hum} | (no Cas9/gRNA delivered) |
| Gaa^{-/-} episomal | Gaa^{-/-}/ | Positive control, previously |
| AAV8 TTR | Tfrc^{hum} | shown rescue of glycogen storage |
| 12847scfv:hGAA | | phenotype |
| Gaa^{-/-} insertion | Gaa^{-/-}/ | Experimental insertion group |
| 12847scfv:hGAA | Tfrc^{hum} | |
| Gaa^{-/-} untreated | Gaa^{-/-}/ | Untreated Pompe disease mouse |
| | Cd63^{hum} | (CD63 humanized) |
| Gaa^{-/-} insertion | Ga^{-/-}/ | Negative control for BBB-crossing |
| anti-CD63scfv:hGAA | Cd63^{hum} | (muscle targeted) |

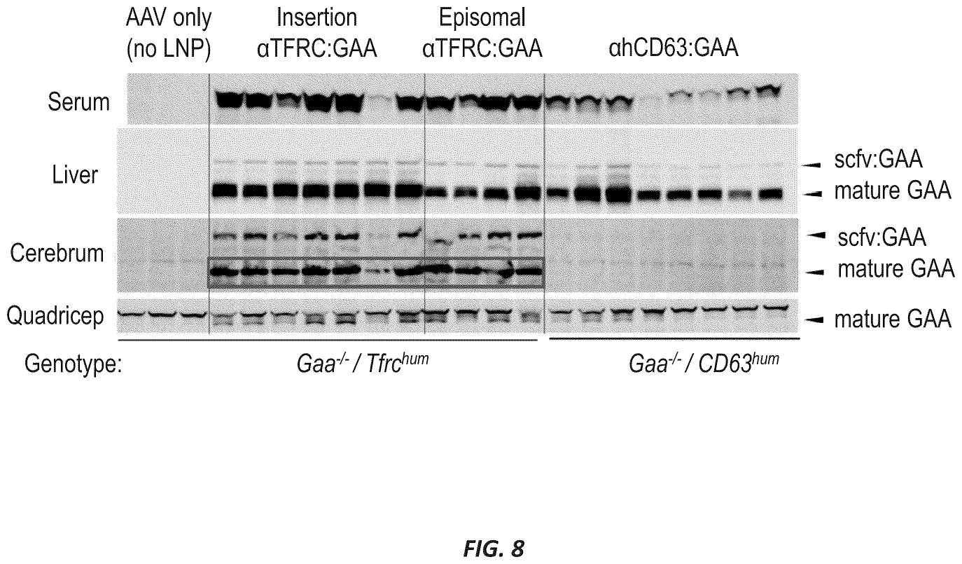

### Western Blot: (Table 34, FIG. 8)

Tissue lysates were prepared by lysis in RIPA buffer with protease inhibitors (1861282, Thermo Fisher, Waltham, MA, USA). Tissue lysates were homogenized with a bead homogenizer (FastPrep5, MP Biomedicals, Santa Ana, CA, USA). Cells or tissue lysates were run on SDS-PAGE gels using the Novex system (LifeTech Thermo, XPO4200BOX, LC2675, LC3675, LC2676). Gels were transferred to low-fluorescence polyvinylidene fluoridev (PVDF) membrane (IPFLO7810, LI-COR, Lincoln, NE, USA) and stained with Revert 700 Total Protein Stain (TPS; 926-11010 LI-COR, Lincoln, NE, USA), followed by blocking with Odyssey blocking buffer (927-500000, LI-COR, Lincoln, NE, USA) in Tris buffer saline with 0.1% Tween 20 and staining with antibodies against GAA (ab137068, Abcam, Cambridge, MA, USA), or anti-GAPDH (ab9484, Abcam, Cambridge, MA, USA) and the appropriate secondary (926-32213 or 925-68070, LI-COR, Lincoln, NE, USA). Blots were imaged with a LI-COR Odyssey CLx.

Protein band intensity was quantified in LI-COR Image Studio software. The quantification of the mature 77 kDa GAA band for each sample was determined by normalizing to the lane's TPS signal (loading control).

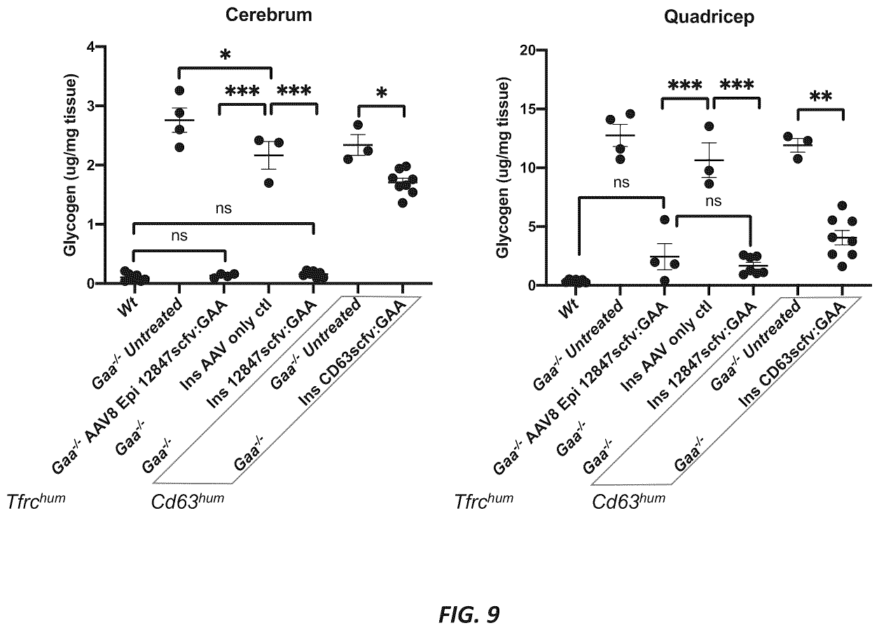

### Glycogen Quantification: (Table 35, FIG. 9)

Tissues were dissected from mice immediately after sacrifice by CO₂ asphyxiation, snap frozen in liquid nitrogen, and stored at −80° C. Tissues were lysed on a benchtop homogenizer with stainless steel beads in distilled water for glycogen measurements or RIPA buffer for protein analyses. Glycogen analysis lysates were boiled and centrifuged to clear debris. Glycogen measurements were performed fluorometrically with a commercial kit according to manufacturer's instructions (K646, BioVision, Milpitas, CA, USA).

TABLE 34
Quantification of hGAA protein in tissues of Gaa^{-/-}/
Tfrc^{hum/hum} mice treated with insertion anti-hTFRC 12847scfv:hGAA

| | Liver | Serum | Cerebrum | Quadricep |
|---|---|---|---|---|
| | total | total | mature | mature |
| Treatment group | hGAA | hGAA | hGAA | hGAA |
| Gaa^{-/-} insertion AAV only negative control | 0.02 ± 0.003 | 0.03 ± 0.02 | 0.002 ± 0.001 | 0.006 ± 0.002 |
| Gaa^{-/-} episomal AAV8 TTR 12847scfv:hGAA | 2.35 ± 0.72 | 3.65 ± 2.09 | 0.49 ± 0.20^{§§} | 0.148 ± 0.043^{§§} |
| Gaa^{-/-} insertion 12847scfv:hGAA | 4.31 ± 0.87* | 3.47 ± 2.37 | 0.57 ± 0.26^{§§} | 0.141 ± 0.062^{§§} |
| Gaa^{-/-} insertion anti-CD63scfv:hGAA | 2.67 ± 1.04* | 0.93 ± 0.55* | 0.01 ± 0.003 | 0.060 ± 0.037 |

All values are arbitrary units, mean ± SD, n = 3-8 per group.
One Way ANOVA
*p < 0.05 vs. Gaa^{-/-} episomal AAV8 TTR 12847scfv:GAA group;
^{§§}p < 0.001 vs. AAV only negative control group.

TABLE 35
Quantification of glycogen in tissues of Gaa^{-/-}/Tfrc^{hum/hum}
mice treated with insertion anti-hTFRC 12847scfv:hGAA

| Treatment group | Cerebrum | Quadricep |
|---|---|---|
| Wt untreated | 0.10 ± 0.07 | 0.37 ± 0.13 |
| Gaa^{-/-}/Tfrc^{hum} untreated (Tfrc^{hum}) | 2.76 ± 0.41 | 12.75 ± 1.88 |
| Gaa^{-/-}/Tfrc^{hum} insertion AAV only | 2.17 ± 0.40* | 10.64 ± 2.56 |
| Gaa^{-/-}/Tfrc^{hum} episomal AAV8 TTR 12847scfv:hGAA | 0.13 ± 0.03***^{§} | 2.44 ± 2.21***^{§} |
| Gaa^{-/-}/Tfrc^{hum} insertion 12847scfv:hGAA | 0.16 ± 0.05***^{§} | 1.67 ± 0.76***^{§} |
| Gaa^{-/-}/Cd63^{hum} untreated | 2.34 ± 0.30 | 11.91 ± 1.01 |
| Gaa^{-/-}/Cd63^{hum} insertion anti-CD63scfv:hGAA | 1.71 ± 0.20* | 4.06 ± 0.13** |

All values are glycogen ug/mg tissue, mean ± SD, n = 3-8 per group.
One Way ANOVA
*p < 0.01 vs. Gaa^{-/-}/Cd63^{hum} untreated group;
**p < 0.001 vs. Gaa^{-/-}/Cd63^{hum} untreated group;
***p < 0.0001 vs. Gaa^{-/-}/Tfrc^{hum/hum} untreated group;
^{§}non-significant vs. Wt untreated group.

### Example 9. Anti-hTFRC:GAA Gene Insertion in Cynomolgus Monkeys

### mAb Clone IDs

H1H12847B in scfv:GAA format (REGN16826)
12450NVH in scfv:GAA format (comparator, REGN5534)

### Insertion of Anti-hTFRC 12847Scfv:GAA in Cynomolgus Monkeys

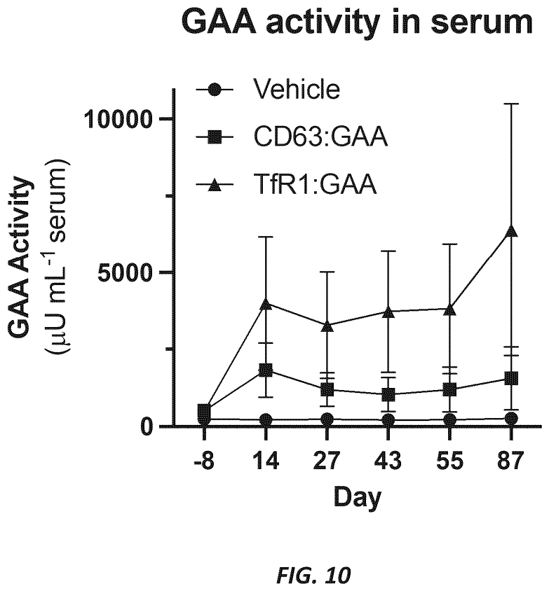

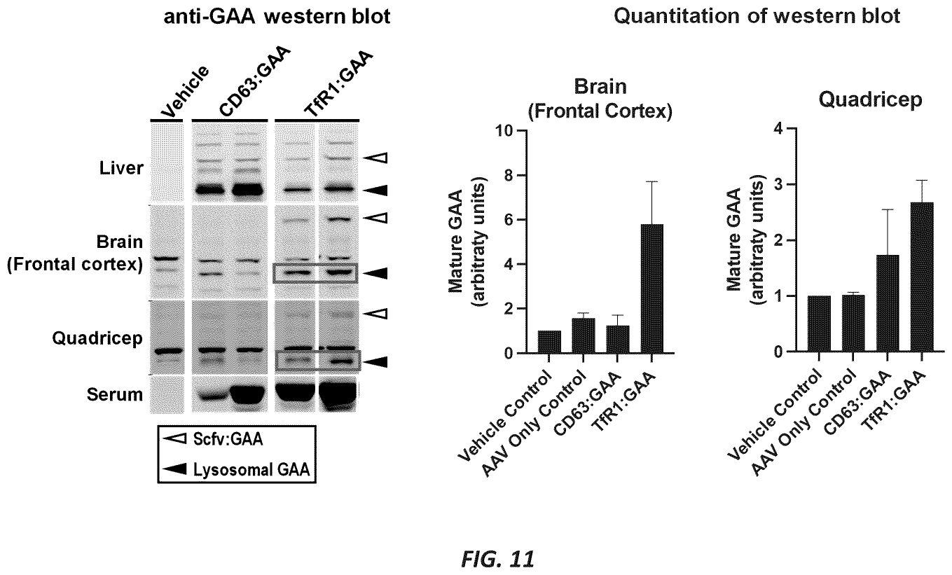

We tested our lead anti-hTFRC 12847scfv:GAA in cynomolgus monkeys by albumin insertion to determine whether we could replicate the results we saw in mice. We compared 12847scfv:GAA to the muscle-targeted anti-hCD63scfv:GAA in cynomolgus monkeys. As shown in FIGS. 10-11, serum GAA activity corresponded to serum GAA protein levels. As shown in FIG. 11, albumin insertion of 12847scfv:GAA delivered mature hGAA protein to the brain (frontal cortex) and muscle (quadricep).

Albumin insertion of anti-hCD63scfv:GAA or 12847scfv:GAA resulted in similar serum GAA levels with two different gRNAs, regardless of what gRNA was used (data not shown). Insertion did not negatively affect serum iron panel or creatinine (data not shown).

### AAV Production

A promoterless AAV genome plasmid was created with the 12847scfv:GAA sequence and the mouse albumin exon 1 splice acceptor site at the 3′ end. Recombinant AAV8 (AAV2/8) was produced in HEK293 cells. Cells were transfected with three plasmids encoding adenovirus helper genes, AAV8 rep and cap genes, and recombinant AAV genomes containing transgenes flanked by AAV2 inverted terminal repeats (ITRs). On day 5, cells and medium were collected, centrifuged, and processed for AAV purification. Cell pellets were lysed by freeze-thaw and cleared by centrifugation. Processed cell lysates and medium were overlaid onto iodixanol gradients columns and centrifuged in an ultracentrifuge. Virus fractions were removed from the interface between the 40% and 60% iodixanol solutions and exchanged into 1×PBS with desalting columns. AAV vg were quantified by ddPCR.

In Vivo CRISPR1Cas9 Insertion into the Albumin Locus

Cynomolgus monkeys age 2-3 years were dosed intravenously with 1.5e13 vg/kg AAV8 12847scfv:GAA (or anti-CD63scfv:GAA) and 3 mg/kg LNP gRNA/Cas9 mRNA. Negative control monkeys received insertion AAV8 without LNP or vehicle control only. Serum and flash-frozen tissues were collected 90 days post-injection.

### GAA Activity in Serum: (FIG. 10)

Serum was collected prior to dosing and at indicated timepoints post-injection. GAA activity in the serum was quantified using Lysosomal alpha-Glucosidase Activity Assay Kit (Abcam ab252887). Serum GAA activity in CD63scfv:GAA and 12847scfv:GAA treated animals was above the vehicle controls and activity was similar between the treatment groups. Serum GAA activity corresponded with liver GAA protein expression and serum GAA protein levels (FIG. 11).

### Western Blot: (FIG. 11)

Tissue lysates were prepared by lysis in RIPA buffer with protease inhibitors (1861282, Thermo Fisher, Waltham, MA, USA). Tissue lysates were homogenized with a bead homogenizer (FastPrep5, MP Biomedicals, Santa Ana, CA, USA). Cells or tissue lysates were run on SDS-PAGE gels using the Novex system (LifeTech Thermo, XP04200BOX, LC2675, LC3675, LC2676). Gels were transferred to low-fluorescence polyvinylidene fluoridev (PVDF) membrane (IPFL07810, LI-COR, Lincoln, NE, USA) and stained with Revert 700 Total Protein Stain (TPS; 926-11010 LI-COR, Lincoln, NE, USA), followed by blocking with Odyssey blocking buffer (927-500000, LI-COR, Lincoln, NE, USA) in Tris buffer saline with 0.1% Tween 20 and staining with antibodies against GAA (ab137068, Abcam, Cambridge, MA, USA), or anti-GAPDH (ab9484, Abcam, Cambridge, MA, USA) and the appropriate secondary (926-32213 or 925-68070, LI-COR, Lincoln, NE, USA). Blots were imaged with a LI-COR Odyssey CLx.

Protein band intensity was quantified in LI-COR Image Studio software. The quantification of the mature 77 kDa GAA band for each sample was determined by normalizing to the lane's TPS signal (loading control).

### Example 10. Optimized Anti-TfR:GAA DNA Templates

Optimized anti-TfR:GAA templates were designed and generated. To select a development cantidate, several versions of the four cantidate anti-TfR:GAA insertion templates were generated in which the nucleotide sequence encoding the anti-TfR:GAA is modified (e.g., by depleting CpGs). Tables 36 and 37 list the different versions of anti-TfR:GAA inserts designed. Each of the anti-TfR:GAA inserts in Table 37 use the optimized GAA sequence set forth in SEQ ID NO: 649.

TABLE 36
Anti-TfR:GAA Inserts for Insertion Cassettes.

| Anti-TfR:GAA Insert | CpGs | SEQ ID NO (optimized GAA) |
|---|---|---|
| 12799 - DC | 0 | 651 |
| 12799 - GS | 0 | 652 |
| 12799 - 1^{st} generation | 160 | 648 |
| 12839 - DC | 0 | 653 |
| 12839 - GS | 0 | 654 |
| 12839 - 1^{st} generation | 163 | 648 |
| 12843 - DC | 0 | 655 |
| 12843 - GS | 0 | 656 |
| 12843 - 1^{st} generation | 160 | 648 |
| 12847 - DC | 0 | 657 |
| 12847 - GS | 0 | 658 |
| 12847 - 1^{st} generation | 160 | 648 |

TABLE 37
Anti-TfR:GAA Inserts for Insertion Cassettes.

| Anti-TfR:GAA Insert | CpGs in Transgene | SEQ ID NO |
|---|---|---|
| 12799 1^{st} generation | 160 | 659 |
| 12799 GA 0 | 0 | 663 |
| 12799 GS 0 | 0 | 664 |
| 12799 GS 0v2 | 0 | 665 |
| 12843 1^{st} generation | 160 | 661 |
| 12843 GA 0 | 0 | 666 |
| 12843 GS 0 | 0 | 667 |
| 12843 GS 0v2 | 0 | 668 |
| 12847 1^{st} generation | 160 | 662 |
| 12847 GA 0 | 0 | 669 |
| 12847 GS 0 | 0 | 670 |
| 12847 GS 0v2 | 0 | 671 |

Peripheral blood mononuclear cells (PBMCs) are isolated from human blood. Plasmacytoid dendritic cells (pDCs) are enriched and combined with pBMCs (1e4 pDCs+1e5 PBMCs per well). The cells are incubated for 16-18 hours with AAV or control CpG-oligodeoxynucleotides (ODNs). The supernatants are harvested, and an IFNα ELISA is performed. This assay assesses whether CpG-depleted anti-TfR:GAA sequences exhibit reduced IFN—I responses in a primary human plasmacytoid DC-based assay as compared to non-CpG-depleted sequences.

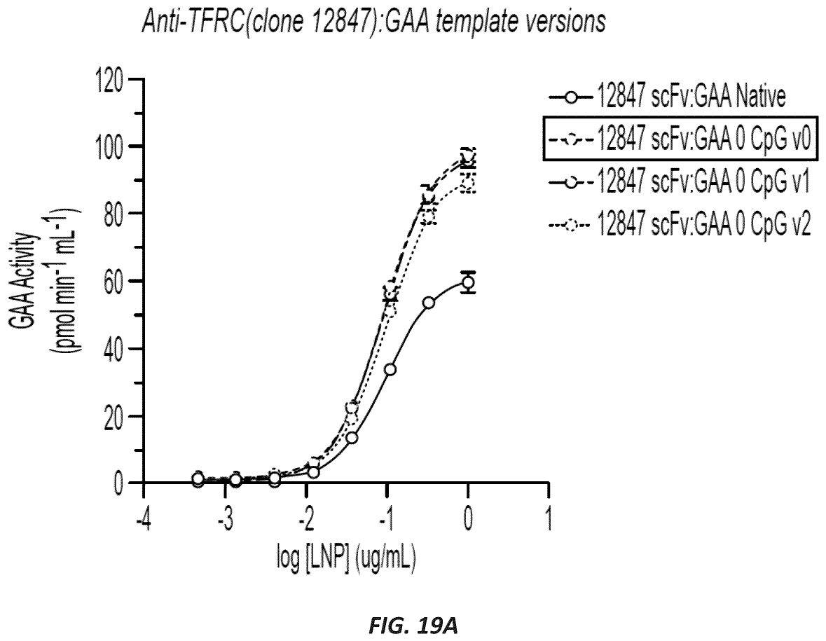

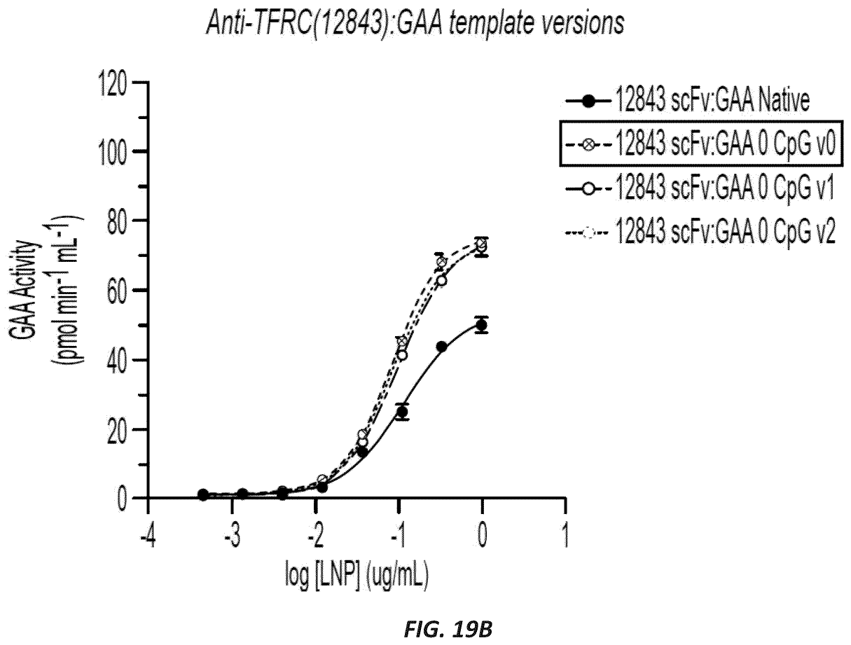

Activity of the various 12847 optimized anti-TfR:GAA templates (SEQ ID NOS: 675-678 or 669-671 (coding sequences)) and 12843 optimized anti-TfR:GAA templates (SEQ ID NOS: 672-674 or 666-668 (coding sequences)) was tested in a primary human hepatocyte assay. AAV templates were packaged into AAV2 viruses. Primary human hepatocytes were grown in 96-well plates and administered the AAV containing the template DNA and LNP-g9860 at fixed MOI (6e4) with LNP dose titration. Supernatants were collected 7 days post-dosing and stored at −80 degrees Celsius. Supernatants were thawed and GAA activity in the supernatants was measured using a 4-methylumbelliferone-based fluorometric assay (K690, BioVision, Milpitas, CA, USA) as a measurement of amount of enzymatically active GAA produced and secreted from the cells. As shown in FIGS. 19A-19B, all CpG-depleted anti-TfR:GAA templates exhibited increased GAA activity in primary human hepatocyte supernatant compared to the native anti-TfR:GAA templates.

Activity of the optimized templates is tested in a primary human hepatocyte assay. AAV templates are packaged into AAV2 viruses. Primary human hepatocytes are grown in 96-well plates and administered the AAV containing the template DNA and LNP-g9860 at fixed LNP concentration with AAV dose titration. Supernatants are collected 7 days post-dosing and stored at −80 degrees Celsius. Supernatants are thawed and GAA activity in the supernatants is measured using a 4-methylumbelliferone-based fluorometric assay (K690, BioVision, Milpitas, CA, USA) as a measurement of amount of enzymatically active GAA produced and secreted from the cells.

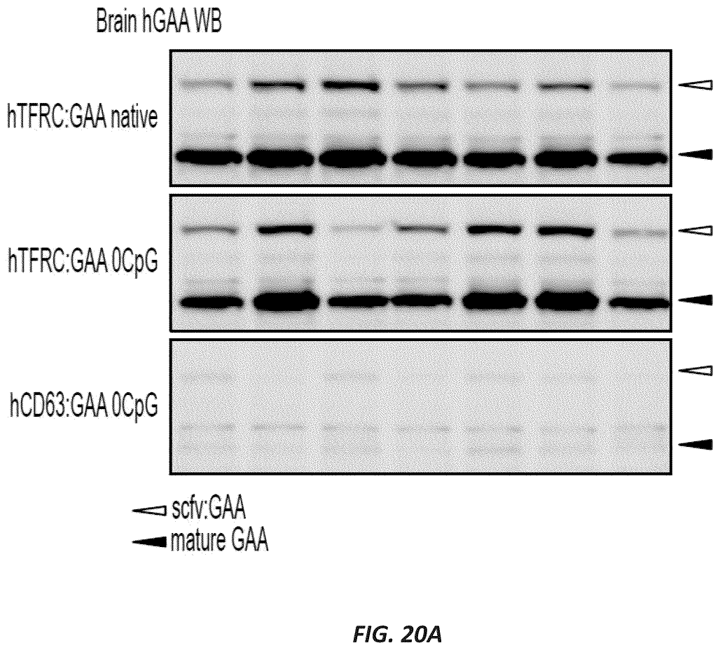

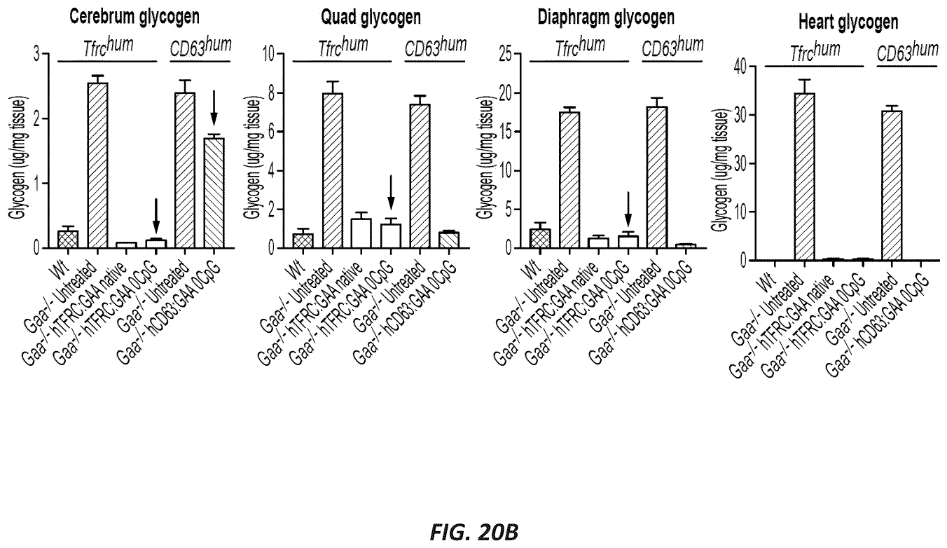

Activity of the 12847 scFv:GAA 0 CpG v0 optimized template (SEQ ID NO: 676 or 669 (coding sequence)) was then validated in the PD mouse model, Gaa^{-/-};Tfrc^{hu/hu} as described in Example 6. Three-month old mice (Gaa^{-/-};Tfrc^{hu/hu} mice and Gaa^{-/-};CD63^{hu/hu} mice) were dosed intravenously with 3 mg/kg LNP-g9860 and 3ev12 vg/kg AAV8 anti-TfR:GAA templates (native or 12847 0 CpG v0) and optimized anti-CD63:GAA template (GA 0 CpG anti-CD63:GAA template; SEQ ID NO: 679 or 650 (coding sequence)), respectively. Western blots for GAA (scFv:GAA and mature GAA) were done as in Example 6 and confirmed delivery of GAA to the brain (cerebrum) following albumin insertion of the native anti-TfR:GAA template or the 0 CpG anti-TfR:GAA template (FIG. 20A). Glycogen quantification in cerebrum, quadriceps, diaphragm, and heart was also done as in Example 6 and confirmed that albumin insertion of the 0 CpG anti-TfR:GAA templates retained TfR binding and GAA activity in vivo and that the CpG depleted sequence was as effective as the native sequence at rescuing the glycogen storage phenotype in Gaa^{-/-};Tfrc^{hu/hu} mice (FIG. 20B and Table 38).

TABLE 38
Quantification of glycogen in tissues of Gaa^{-/-}/Tfrc^{hum}
mice treated with anti-hTfRscfv:hGAA insertion templates.

| Treatment group | Cerebrum | Diaphragm | Heart | Quadricep |
|---|---|---|---|---|
| hTFRC Wt | 0.256 ± 0.175* | 2.44 ± 1.97* | 0.042 ± 0.022* | 0.724 ± 0.611* |
| hTFRC Gaa−/− Untreated | 2.56 ± 0.237 | 17.54 ± 1.72 | 34.56 ± 6.36 | 7.96 ± 1.38 |
| hTFRC Gaa^{-/-} 12847:GAA native | 0.076 ± 0.022* | 1.30 ± 0.973* | 0.405 ± 0.351* | 1.48 ± 0.867* |
| hTFRC Gaa^{-/-}12847:GAA 0 CpG | 0.114 ± 0.049* | 1.55 ± 1.60* | 0.35 ± .372* | 1.21 ± 0.821* |
| hCD63 Gaa^{-/-} Untreated | 2.39 ± 0.432 | 18.30 ± 2.62 | 30.88 ± 2.451 | 7.37 ± 1.134 |
| hCD63 Gaa^{-/-} CD63:GAA 0 CpG | 1.68 ± 0.220* | 0.544 ± 0.294* | 0.0634 ± 0.045* | 0.803 ± 0.297* |

All values are glycogen μg/mg tissue, mean ± SD, n = 5-8 per group.
One Way ANOVA
*p < 0.0001 vs. hTFRC Gaa^{-/-} Untreated group.

### Example 11. Epitope Mapping for Transferrin (TfR) Antibodies

Hydrogen-Deuterium Exchange Mass Spectrometry (HDX-MS) was performed to delineate regions in mouse and human Transferrin (m/hTfR) involved in binding of anti-Transferrin Receptor (TfR) antibodies. The anti-TfR monoclonal antibodies tested are described in Table 39. The reagents used and corresponding lot numbers are set forth in Table 40.

TABLE 39
Monoclonal Antibody Clones Tested

| REGN# | AbPID | Lot # |
|---|---|---|
| REGN17507 | H1H12798B | L1 |
| REGN17508 | H1H12799B | L1 |
| REGN17509 | H1H12835B | L1 |
| REGN17510 | H1H12839B | L1 |
| REGN17511 | H1H12841B | L1 |
| REGN17512 | H1H12843B | L1 |
| REGN17513 | H1H12845B | L1 |
| REGN17514 | H1H12847B | L1 |
| REGN17515 | H1H12848B | L1 |
| REGN17516 | H1H12850B | L1 |
| REGN17517 | H1H31874B | L1 |

TABLE 40
Reagents Used and Lot Numbers

| REGN# | Lot # | Description |
|---|---|---|
| REGN2120 | 03-121015 | hTfR(C89-F763).mmh |
| REGN2431 | L1 | hmm.hTfR(C89-F763) |

A general description of the HDX-MS method is set forth in, e.g., Ehring (1999) Analytical Biochemistry 267(2):252-259; and Engen and Smith (2001) Anal. Chem. 73:256A-265A. The experiment was performed on a customized HDX automation system (NovaBioAssays, MA) coupled to a Q Exactive HF mass spectrometer (Thermno Fisher Scientific, MA).

PBS-D₂O buffer was prepared by dissolving one PBS tablet in 100 mL 99.9% D₂O to form solution of 10 mM sodium phosphate, 137 mM NaCl, 3 mM KCl, pD 7.0 (equivalent to pH 7.4 at 25° C.). To initiate deuterium exchange, 10 μL of protein sample (hTfR alone, or hTfR in mixture with either of the monoclonal mAbs listed above, see, e.g., Table 39) was diluted with 90 μL PBS-D₂O buffer. After 5 minutes or 10 minutes, deuterium exchange was quenched by adding 100 μL quenching buffer (0.5 M TCEP, 4 M guanidine hydrochloride, pH 2.08) followed by 90 second incubation at 20° C. The quenched samples were digested by online pepsin/protease XIII column (NovaBioAssays, MA) at room temperature with 100 μL/min 0.1% formic acid in water. Peptic peptides were trapped by an ACQUITY UPLC Peptide BEH C18 VanGuard Pre-column (2.1×5 mm, Waters, MA) and further separated by an ACQUITY UPLC Peptide BEH C18 column (2.1×50 mm, Waters, MA) at −5° C., using 10-minute or 15-minute gradients with 0.1% formic acid in water and 0.1% formic acid in acetonitrile as mobile phases at 200 μL/min. Eluted peptides were analyzed by the mass spectrometer in LC-MS/MS or LC-MS mode.

A set of non-deuterated samples was prepared in PBS—H₂O buffer and analyzed with the method described above to identify peptide sequences and determine peptide masses without deuterium exchange. The LC-MS/MS data of non-deuterated samples were searched against a database containing sequences of hTfR, pepsin and protease XIII using the Byonic search engine (Protein Metrics, CA) with parameters for non-specific enzymatic digestion. The identified peptide list was then imported into the HDExaminer software (Sierra Analytics, CA) together with LC-MS data from all deuterated samples to calculate the deuterium uptake percentage (D %) of individual peptides from hTfR. Differences in deuterium uptake were calculated as ΔD %=D % of hTfR-mAb−D % of hTfR. Differences were considered significant if ΔD %<−5% (equivalent to |ΔD|>5% and ΔD %<0, averaged from 2 replicates). Mass spectra of peptides showing significant differences were examined manually to ensure that correct isotopic patterns were used for D % calculations by the software.

Two TfR protein constructs were used in HDX-MS experiments by reason of reagent availability and antibody specificity: hTfR(C89-F763).mmh, and hmm.hTfR(C89-F763). HDX data were obtained on 88%-95% of amino acids in hTfR with mmh tag. The numerical range provided before each amino acid sequence in the list below indicates the amino acid (aa) residue positions in hTfR which are protected by the indicated antibody. These amino acid residue positions are indicative of antibody binding sites on hTfR and does not provide residue-level contacts between them. Due to the nature of HDX-MS technique, the regions obtained by HDX-MS may be larger or smaller than actual contacts determined by high-resolution structural studies such as X-ray crystallography and cryogenic electron microscopy methods.

REGN17507 (H1H12798B) protects the following
regions in hTfR:
146-167
(SEQ ID NO: 704)
LLNENSYVPREAGSQKDENLAL;
281-295
(SEQ ID NO: 705)
IYMDQTKFPIVNAEL;
and
572-576
(SEQ ID NO: 706)
TYKEL.
REGN17508 (H1H12799B) protects the following
regions in hTfR:
128-146
(SEQ ID NO: 707)
KRKLSEKLDSTDFTGTIKL;
503-522
(SEQ ID NO: 708)
YTLIEKTMQNVKHPVTGQFL;
and
576-592
(SEQ ID NO: 709)
LIERIPELNKVARAAAE.
REGN17509 (H1H12835B) protects the following
region in hTfR:
147-165
(SEQ ID NO: 710)
LNENSYVPREAGSQKDENL.
REGN17510 (H1H12839B) protects the following
region in hTfR:
238-246
(SEQ ID NO: 711)
GTKKDFEDL.
REGN17511 (H1H12841B) protects the following
region in hTfR:
199-224
(SEQ ID NO: 712)
SVIIVDKNGRLVYLVENPGGYVAYSK.
REGN17512 (H1H12843B) protects the following
regions in hTfR:
146-164
(SEQ ID NO: 713)
LLNENSYVPREAGSQKDEN;
284-295
(SEQ ID NO: 714)
DQTKFPIVNAEL;
and
572-585
(SEQ ID NO: 715)
TYKELIERIPELNK.
REGN17513 (H1H12845B) protects the following
region in hTfR:
199-222
(SEQ ID NO: 716)
SVIIVDKNGRLVYLVENPGGYVAY.
REGN17514 (H1H12847B) protects the following
regions in hTfR:
146-164
(SEQ ID NO: 713)
LLNENSYVPREAGSQKDEN;
and
572-585
(SEQ ID NO: 715)
TYKELIERIPELNK.
REGN17515 (H1H12848B) protects the following
regions in hTfR:
281-295
(SEQ ID NO: 705)
IYMDQTKFPIVNAEL;
and
346-365
(SEQ ID NO: 717)
FGNMEGDCPSDWKTDSTCRM.
REGN17516 (H1H12850B) protects the following
regions in hTfR:
146-167
(SEQ ID NO: 704)
LLNENSYVPREAGSQKDENLAL;
212-232
(SEQ ID NO: 719)
LVENPGGYVAYSKAATVTGKL;
281-297
(SEQ ID NO: 720)
IYMDQTKFPIVNAELSF;
337-345
(SEQ ID NO: 721)
ISRAAAEKL;
366-383
(SEQ ID NO: 722)
VTSESKNVKLTVSNVLKE;
and
557-572
(SEQ ID NO: 723)
FCEDTDYPYLGTTMDT
REGN17517 (H1H31874B) protects the following
region in hTfR:
243-246
(SEQ ID NO: 718)
FEDL.

The minimal amino acid sequence in hTfR which is protected by the above-listed anti-TfR antibodies (i.e., the minimal epitope sequence), numerical range indicating the amino acid (aa) residue positions in hTfR which are protected each antibody, as well as the conformational or linear nature of each minimal epitope are described in Table 41. Each of the minimal epitopes is bound by its corresponding antibody at one or more amino acid residues within the minimal epitope sequence.

TABLE 41
Minimal epitope sequences in hTfR protected by
anti-TfR antibodies.

| | | | Amino | | |
|---|---|---|---|---|---|
| | Epi- | | acid | Amino | SEQ |
| Antibody | tope | | residue | acid | ID |
| ID | No. | Class | positions | sequence | NO |
| REGN17507 | 1 | conforma- | 146-149 | LLNE | 796 |
| (H1H12798B) | | tional | | | |
| REGN17507 | 2 | conforma- | 572-576 | TYKEL | 706 |
| (H1H12798B) | | tional | | | |
| REGN17508 | 1 | conforma- | 136-143 | DSTDFTGT | 797 |
| (H1H12799B) | | tional | | | |
| REGN17508 | 2 | conforma- | 513-521 | VKHPVTGQF | 798 |
| (H1H12799B) | | tional | | | |
| REGN17508 | 3 | conforma- | 577-583 | IERIPEL | 799 |
| (H1H12799B) | | tional | | | |
| REGN17509 | 1 | linear | 147-164 | LNENSYVPRE | 800 |
| (H1H12835B) | | | | AGSQKDEN | |
| REGN17510 | 1 | linear | 243-246 | FEDL | 718 |
| (H1H12839B) | | | | | |
| REGN17511 | 1 | linear | 202-209 | IVDKNGRL | 801 |
| (H1H12841B) | | | | | |
| REGN17512 | 1 | conforma- | 146-149 | LLNE | 796 |
| (H1H12843B) | | tional | | | |
| REGN17512 | 2 | conforma- | 572-576 | TYKEL | 706 |
| (H1H12843B) | | tional | | | |
| REGN17513 | 1 | linear | 202-211 | IVDKNGRLVY | 802 |
| (H1H12845B) | | | | | |
| REGN17514 | 1 | conforma- | 146-149 | LLNE | 796 |
| (H1H12847B) | | tional | | | |
| REGN17514 | 2 | conforma- | 572-576 | TYKEL | 706 |
| (H1H12847B) | | tional | | | |
| REGN17515 | 1 | linear | 284-288 | DQTKF | 803 |
| (H1H12848B) | | | | | |
| REGN17516 | 1 | conforma- | 212-218 | LVENPGGY | 804 |
| (H1H12850B) | | tional | | | |
| REGN17516 | 2 | conforma- | 289-297 | PIVNAELSF | 805 |
| (H1H12850B) | | tional | | | |
| REGN17516 | 3 | conforma- | 564-572 | PYLGTTMDT | 806 |
| (H1H12850B) | | tional | | | |
| REGN17517 | 1 | linear | 243-246 | FEDL | 718 |
| (H1H31874B) | | | | | |

The extracellular unit of hTfR is structurally categorized into three domains, the helical, protease-like and apical domains (PDB 1SUV).

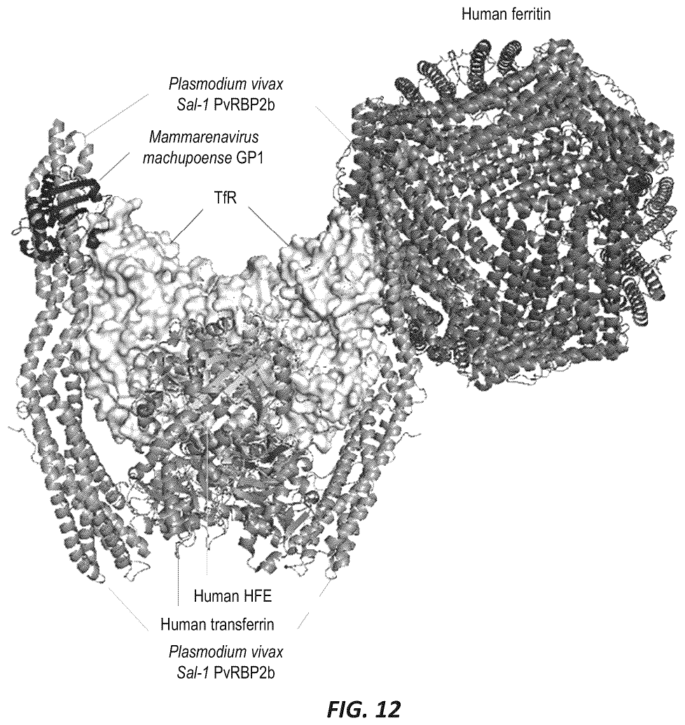

Structural studies of TfR in complex with a variety of molecules that have identified TfR binding sites, including Mammarenavirus machupoense GP1 protein (PDB 3KAS), canine parvovirus (PDB 2NSU), human ferritin (PDB 6GSR), Plasmodium vivax Sal-1 PvRBP2b (PDB 6D04), human HFE protein (PDB 1DE4), human transferrin (PDB 1SUV), etc. FIG. 12 shows the interactions of the above-listed molecules superimposed on a single TfR molecule.

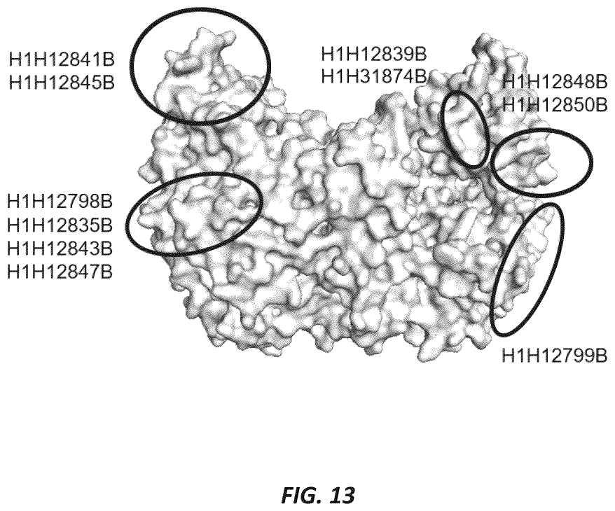

HDX protections for the antibodies tested in HDX-MS experiments can be assigned to 5 regions in TfR (PDB 1SUV) as depicted in FIG. 13.

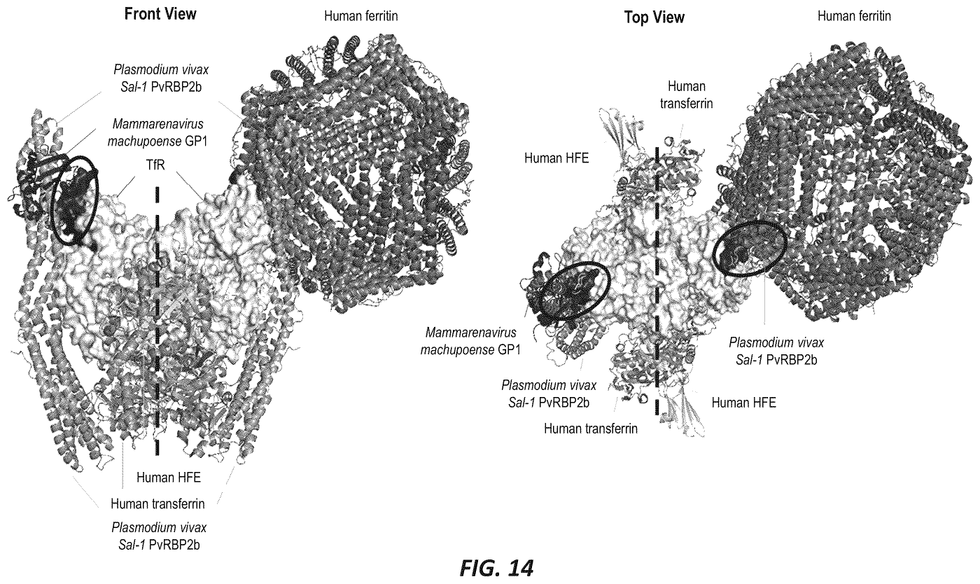

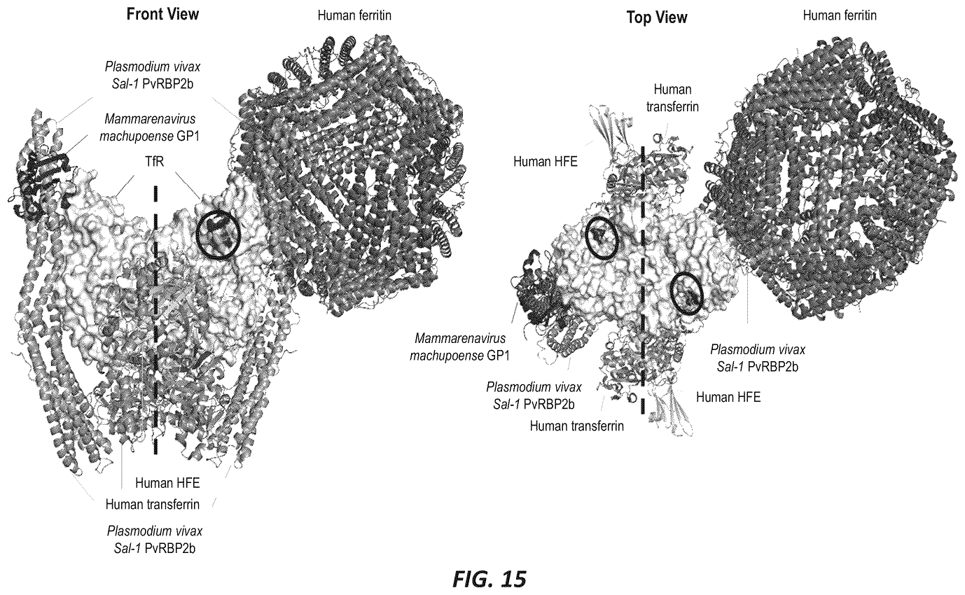

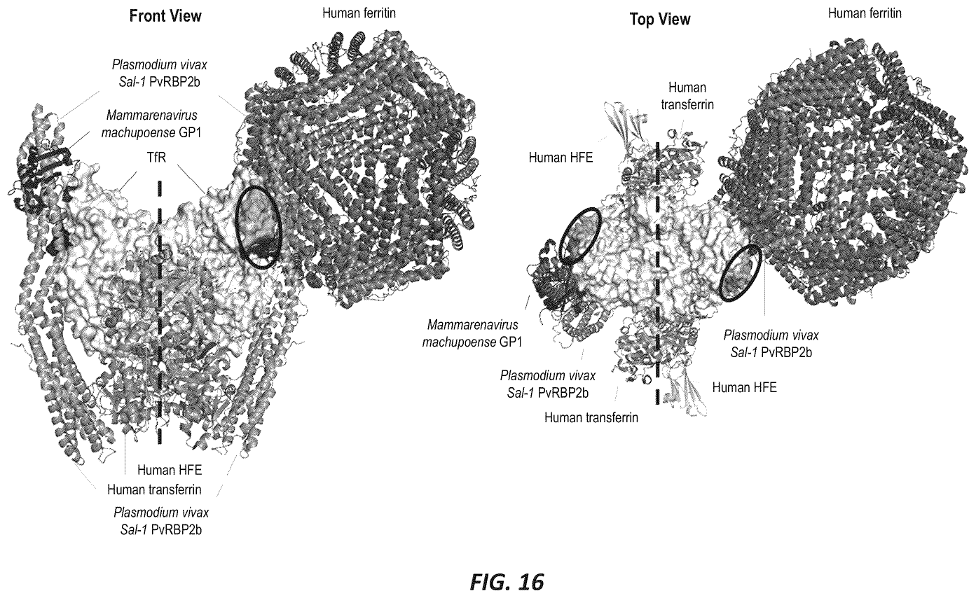

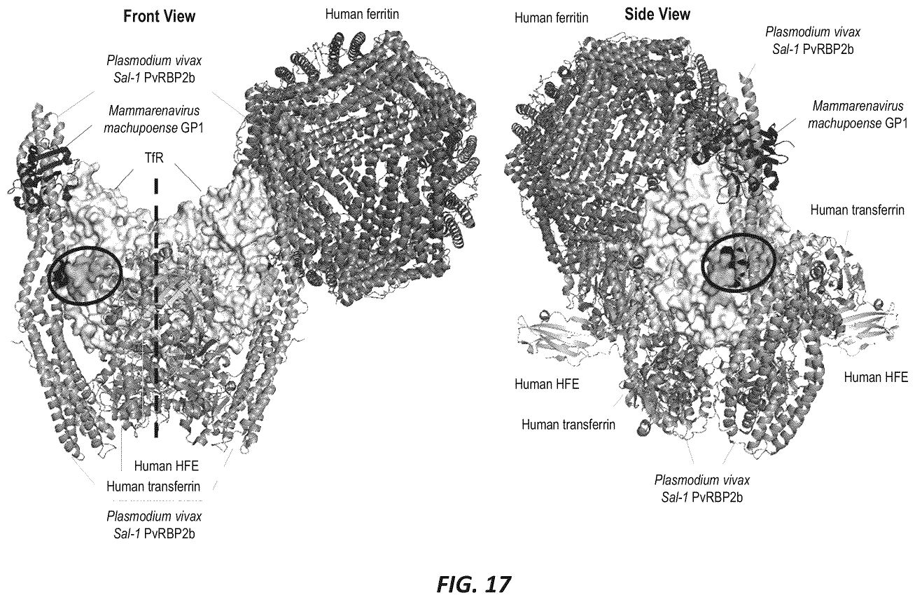

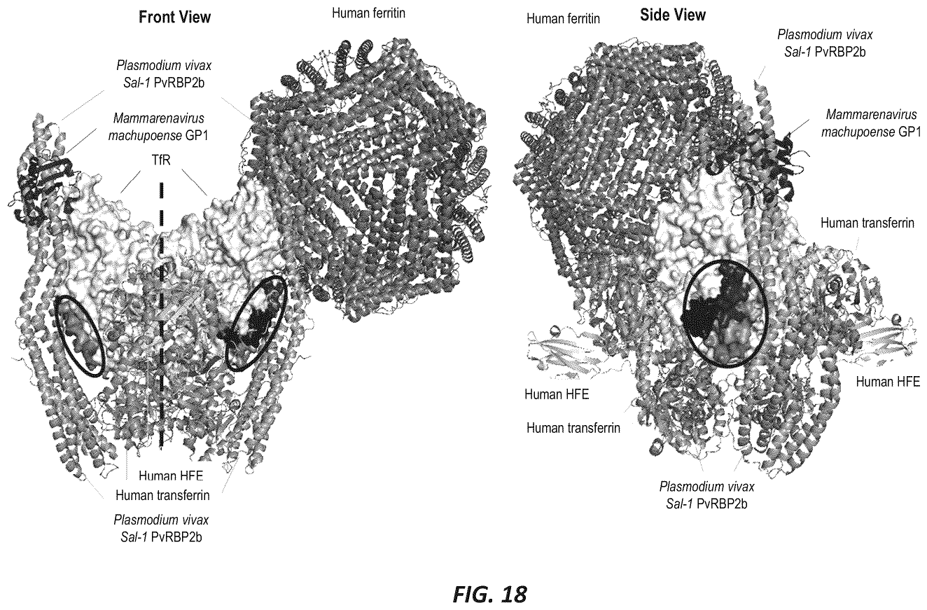

Tabulated summaries of data of the present Example are described in Table 42 to Table 46. FIGS. 14-18 correspond to the tables below.

TABLE 42
Antibodies that show HDX protections in TfR
apical domain and overlap with Mammarenavirus
machupoense GP1, canine parvovirus, human
ferritin, and plasmodium vivax Sal-1 PvRBP2b
binding sites.

| | | | m/hTfR | |
|---|---|---|---|---|
| | | | residues | |
| | | | with | |
| | | | significant | |
| | | | changes in | Sequence |
| Antibody | REGN# | Antigen | deuterium % | coverage |
| H1H12841B | REGN17511 | hTfR.mmh | 199-224 | ~92.9% |
| | | | SVIIVDKNGRLVY | |
| | | | LVENPGGYVAYSK | |
| | | | (SEQ ID NO: | |
| | | | 712) | |
| H1H12845B | REGN17513 | hmm.hTfR | 199-222 | ~88.1% |
| | | | SVIIVDKNGRLVY | |
| | | | LVENPGGYVAY | |
| | | | (SEQ ID NO: | |
| | | | 716) | |

TABLE 43
Antibodies with HDX protections in TfR apical
domain that are not shared by other TfR
binding partners listed in Table 42.

| | | | m/hTfR | |
|---|---|---|---|---|
| | | | residues | |
| | | | with | |
| | | | significant | |
| | | | changes in | Sequence |
| Antibody | REGN# | Antigen | deuterium % | coverage |
| H1H31874B | REGN17517 | hTfR.mmh | 243-246 | ~92.9% |
| | | | FEDL | |
| | | | (SEQ ID | |
| | | | NO: 718) | |
| H1H12839B | REGN17510 | hmm.hTfR | 238-246 | ~88.3% |
| | | | GTKKDFEDL | |
| | | | (SEQ ID | |
| | | | NO: 711) | |

TABLE 44
Antibodies with HDX protections in TfR apical
domain that share binding sites with human
ferritin and plasmodium vivax Sal-1 PvRBP2b.

| | | | m/hTfR | |
|---|---|---|---|---|
| | | | residues | |
| | | | with | |
| | | | significant | |
| | | | changes in | Sequence |
| Antibody | REGN# | Antigen | deuterium % | coverage |
| H1H12848B | REGN17515 | hTfR.mmh | 281-295 | ~92.9% |
| | | | IYMDQTKFPIVN | |
| | | | AEL | |
| | | | (SEQ ID | |
| | | | NO: 705) | |
| | | | 346-365 | |
| | | | FGNMEGDCPSDW | |
| | | | KTDSTCRM | |
| | | | (SEQ ID | |
| | | | NO: 717) | |
| H1H12850B | REGN17516 | hTfR.mmh | 146-167 | ~92.9% |
| | | | LLNENSYVPREA | |
| | | | GSQKDENLAL | |
| | | | (SEQ ID | |
| | | | NO: 704) | |
| | | | 212-232 | |
| | | | LVENPGGYVAYS | |
| | | | KAATVTGKL | |
| | | | (SEQ ID | |
| | | | NO: 719) | |
| | | | 281-297 | |
| | | | IYMDQTKFPIVN | |
| | | | AELSF | |
| | | | (SEQ ID | |
| | | | NO: 720) | |
| | | | 337-345 | |
| | | | ISRAAAEKL | |
| | | | (SEQ ID | |
| | | | NO: 721) | |
| | | | 366-383 | |
| | | | VTSESKNVKLTV | |
| | | | SNVLKE | |
| | | | (SEQ ID | |
| | | | NO: 722) | |
| | | | 557-572 | |
| | | | FCEDTDYPYLGT | |
| | | | TMDT | |
| | | | (SEQ ID | |
| | | | NO: 723) | |

TABLE 45
Antibodies with HDX protections in TfR
protease-like domain and share binding
sites with plasmodium vivax Sal-1 PvRBP2b.

| | | | m/hTfR | |
|---|---|---|---|---|
| | | | residues | |
| | | | with | |
| | | | significant | |
| | | | changes in | Sequence |
| Antibody | REGN# | Antigen | deuterium % | coverage |
| H1H12798B | REGN17507 | hmm.hTfR | 146-167 | ~87.0% |
| | | | LLNENSYVPREA | |
| | | | GSQKDENLAL | |
| | | | (SEQ ID | |
| | | | NO: 704) | |
| | | | 281-295 | |
| | | | IYMDQTKFPIVN | |
| | | | AEL | |
| | | | (SEQ ID | |
| | | | NO: 705) | |
| | | | 572-576 | |
| | | | TYKEL | |
| | | | (SEQ ID | |
| | | | NO: 706) | |
| H1H12843B | REGN17512 | hmm.hTfR | 146-164 | ~88.3% |
| | | | LLNENSYVPREA | |
| | | | GSQKDEN | |
| | | | (SEQ ID | |
| | | | NO: 713) | |
| | | | 284-295 | |
| | | | DQTKFPIVNAEL | |
| | | | (SEQ ID | |
| | | | NO: 714) | |
| | | | 572-585 | |
| | | | TYKELIERIPEL | |
| | | | NK | |
| | | | (SEQ ID | |
| | | | NO: 715) | |
| H1H12847B | REGN17514 | hmm.hTfR | 146-164 | ~88.1% |
| | | | LLNENSYVPREA | |
| | | | GSQKDEN | |
| | | | (SEQ ID | |
| | | | NO: 713) | |
| | | | 572-585 | |
| | | | TYKELIERIPEL | |
| | | | NK | |
| | | | (SEQ ID | |
| | | | NO: 715) | |
| H1H12835B | REGN17509 | hTfR.mmh | 147-165 | ~92.9% |
| | | | LNENSYVPREAG | |
| | | | SQKDENL | |
| | | | (SEQ ID | |
| | | | NO: 710) | |

TABLE 46
Antibodies with HDX protections in TfR
protease-like domain. This region is not
utilized by other TfR interacting molecules
listed in Table 45.

| | | | m/hTfR | |
|---|---|---|---|---|
| | | | residues | |
| | | | with | |
| | | | significant | |
| | | | changes in | Sequence |
| Antibody | REGN# | Antigen | deuterium % | coverage |
| H1H12799B | REGN17508 | hmm.hTfR | 128-146 | ~88.7% |
| | | | KRKLSEKLDSTD | |
| | | | FTGTIKL | |
| | | | (SEQ ID | |
| | | | NO: 707) | |
| | | | 503-522 | |
| | | | YTLIEKTMQNVK | |
| | | | HPVTGQFL | |
| | | | (SEQ ID | |
| | | | NO: 708) | |
| | | | 576-592 | |
| | | | LIERIPELNKVA | |
| | | | RAAAE | |
| | | | (SEQ ID | |
| | | | NO: 709) | |

### REFERENCES

1. Ehring (1999) Analytical Biochemistry 267(2):252-259
2. Engen and Smith (2001) Anal. Chem. 73:256A-265A

### Example 12. Durable Alpha-Glucosidase (GAA) Expression after Insertion of Anti-CD63:GAA DNA Template in Neonatal Mice

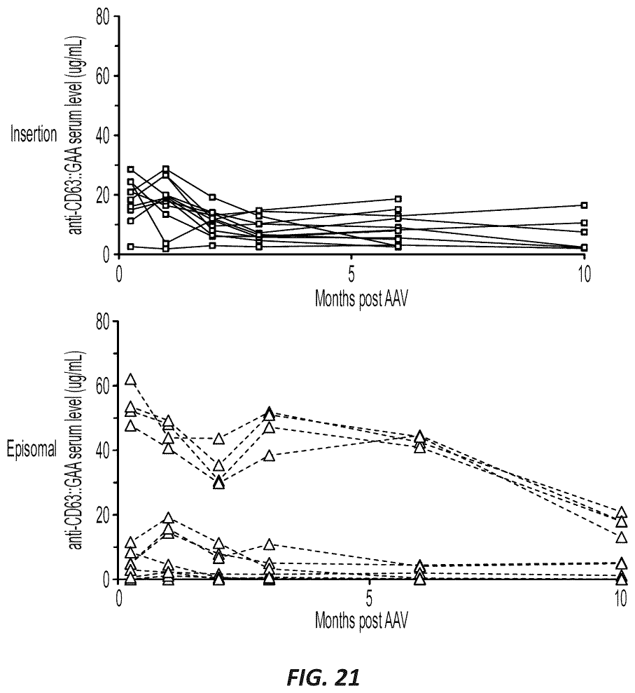

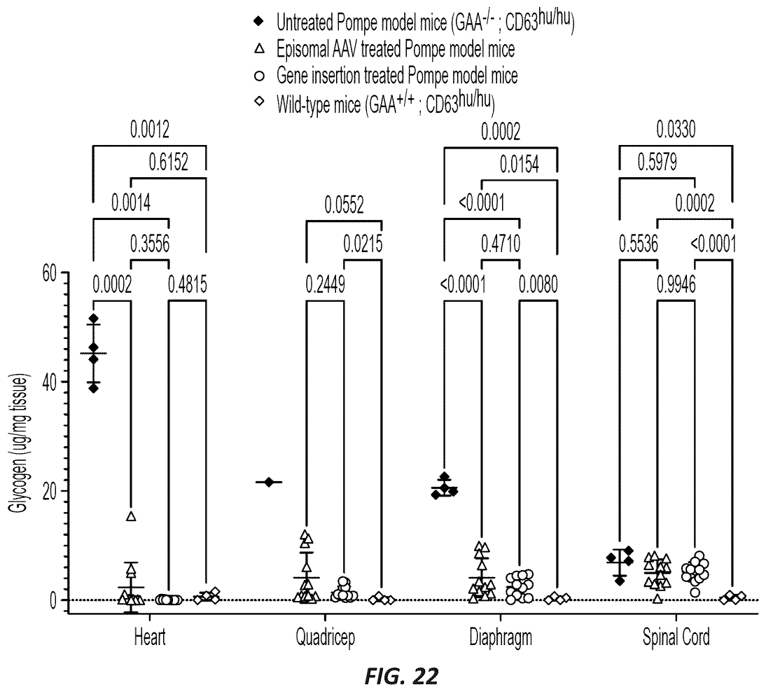

We next engineered a DNA template for insertion of a nucleic encoding anti-CD63:GAA fusions in which the C-terminus of an anti-CD63 single-chain fragment variable (scFv) is fused to the N-terminus of GAA with a glycine-serine linker (described above). We tested the anti-CD63:GAA insertion template in a Pompe disease (PD) mouse model, Gaa^{-/-};Cd63^{hu/hu}, where Gaa was replaced by LacZ and the protein-coding region of the Cd63 locus was replaced with its human counterpart. Adult (2-month old) male and female Gaa^{-/-};Cd63^{hu/hu} mice (62.5% C57BL/6, 37.5% 129Sv) were dosed intravenously with the following: (1) 4e12 vg/kg recombinant AAV8 encoding anti-CD63:GAA (REGV042); or (2) 1 mg/kg LNP-g666 and 1.2e13 vg/kg recombinant AAV8 anti-CD63:GAA insertion template (REGV044). REGV042 is an episomal AAV that uses a hSerpinal enhancer and a mTTR promoter to give hepatocyte-specific expression of anti-CD63:GAA, which further includes a human albumin signal peptide. The anti-CD63:GAA coding sequences were identical in REGV042 and REGV044 and are set forth in SEQ ID NO: 734. Untreated Gaa^{-/-};Cd63^{hu/hu} mice and wild type mice were used as controls. Blood was collected and serum prepared at 7 days, 30 days, 2 months, 3 months, 6 months, and 10 months post-administration, and tissues were collected at 10 months post-administration. Anti-CD63:GAA serum levels were quantified using a plate-based sandwich ELISA that detects the scFv portion of the molecule. Anti-CD63:GAA purified protein was used as a protein standard for quantification. Data are shown in FIG. 21 and Tables 47-48. At 10 months post-administration, animals were sacrificed, and glycogen levels were quantified in muscle tissue lysates of the sacrificed animals. Tissues were dissected from mice immediately after sacrifice by CO₂ asphyxiation, snap frozen in liquid nitrogen, and stored at −80° C. Tissues were lysed on a benchtop homogenizer with stainless steel beads in distilled water for glycogen measurements or RIPA buffer for protein analyses. Glycogen analysis lysates were boiled and centrifuged to clear debris. Glycogen measurements were performed fluorometrically with a commercial kit according to manufacturer's instructions (K646, BioVision, Milpitas, CA, USA). As shown in FIG. 22 and Tables 49-51, glycogen was significantly reduced to near wild type levels in both the episomal group and the insertion group in heart, quadricep, and diaphragm in adult mice.

TABLE 47
Serum Levels of Anti-CD63:GAA in μg/mL in Insertion Adult Group.

| Months | M1 | M2 | M3 | M4 | M5 | F1 | F2 | F3 | F4 | F5 | F6 | F7 |
|---|---|---|---|---|---|---|---|---|---|---|---|---|
| 0.25 | 21.06 | 2.6 | 14.93 | 24.37 | 24.39 | 16.08 | 11.21 | 18.35 | 28.54 | 20.94 | | |
| 1 | 16.43 | 1.85 | 17.92 | 13.45 | 3.7 | 18.88 | 19.37 | 26.6 | 19.8 | 28.72 | 26.63 | 19.97 |
| 2 | 13.15 | 2.95 | 13.82 | 6.06 | 11.51 | 6.45 | 9.5 | 8.05 | 11.61 | 19.2 | 12.07 | 14.09 |
| 3 | 14.49 | 2.53 | 7.22 | 6.04 | 5.75 | 4.62 | 10.19 | 5.75 | 14.8 | 12.93 | 6.61 | 10.26 |
| 6 | 12.92 | 3.13 | 12.08 | 8.18 | 5.54 | 2.48 | 9.08 | 8 | 18.66 | 2.78 | 5.03 | 15.15 |
| 10 | 16.5 | 2 | 7.5 | 10.6 | 2.3 | | 2.4 | | | | | |

*Cells without data were due to lost samples post-collection.

TABLE 48
Serum Levels of Anti-CD63:GAA in μg/mL in Episomal Adult Group.

| Months | M1 | M2 | M3 | M4 | M5 | M6 | M7 | F1 | F2 | F3 | F4 | F5 |
|---|---|---|---|---|---|---|---|---|---|---|---|---|
| 0.25 | 62.13 | 52.18 | 47.7 | 53.55 | 4.9 | 4.85 | 2.92 | 11.55 | 0 | 0 | 8.37 | 0.6 |
| 1 | 43.85 | 48.12 | 40.68 | 49.22 | 14.41 | 15.74 | 2.21 | 19.18 | 0 | 1.09 | 4.57 | 2.25 |
| 2 | 43.68 | 30.59 | 29.79 | 35.37 | 7.88 | 6.71 | 1.67 | 11.27 | 0 | 0.51 | 0.54 | 0 |
| 3 | 51.86 | 47.18 | 38.41 | 50.98 | 5.05 | 10.89 | 1.54 | 3.26 | 0 | 0 | 0.19 | 0.6 |
| 6 | 42.49 | 40.95 | 44.62 | 44.2 | 4.42 | 4.03 | 1.93 | 0.35 | 0 | 0 | 0.17 | 0.17 |
| 10 | 20.9 | 17.9 | 18 | 13.1 | 5.2 | 4.9 | 1.2 | 0.1 | 0 | 0 | 0 | 0 |

TABLE 49
Glycogen Levels in Insertion Adult Group.

| | M1 | M2 | M3 | M4 | M5 | F1 | F2 | F3 | F4 | F5 | F6 | F7 |
|---|---|---|---|---|---|---|---|---|---|---|---|---|
| Heart | 0.02 | 0.02 | 0.02 | 0.01 | 0.22 | 0.02 | 0.02 | 0.19 | 0.04 | 0.02 | 0.04 | 0.02 |
| Quadricep | 0.43 | 3.24 | 0.47 | 0.79 | 2.12 | 0.5 | 2.32 | 0.82 | 1.06 | 3.45 | 0.83 | 0.89 |
| Diaphragm | 2.85 | 1.51 | 0.34 | 2.24 | 4.75 | 0.37 | 4.06 | 0.9 | 4.47 | 4.56 | 2.86 | 0.04 |
| Spinal Cord | 4.28 | 8.11 | 5.82 | 1.38 | 3.41 | 5.46 | 6.68 | 5.63 | 4 | 4.62 | 5.57 | 7.02 |

TABLE 50
Glycogen Levels in Episomal Adult Group.

| | M1 | M2 | M3 | M4 | M5 | M6 | M7 | F1 | F2 | F3 | F4 | F5 |
|---|---|---|---|---|---|---|---|---|---|---|---|---|
| Heart | 0.18 | 0.13 | 0.22 | 0.02 | 0.18 | 0.02 | 0 | 1.04 | 15.39 | 4.99 | 5.74 | 0.13 |
| Quadricep | 0.87 | 0.54 | 0.72 | 0.27 | 0.71 | 0.85 | 2.78 | 6.09 | 12.05 | 10.46 | 11.31 | 2.89 |
| Diaphragm | 2.39 | 1.62 | 3.41 | 1.22 | 2.13 | 0.32 | 0.75 | 6.41 | 9.94 | 9.7 | 8.58 | 2.89 |
| Spinal Cord | 0.3 | 2.63 | 3.37 | 3.19 | 2.96 | 5.99 | 6.1 | 4.66 | 6.46 | 7.9 | 8.15 | 7.67 |

TABLE 51
Glycogen Levels in Control Adult Groups.

| | Untreated Pompe mice (GAA−/−; CD63 hu/hu) | | | | Wild-type (GAA+/+ CD63hu/hu) | | | |
|---|---|---|---|---|---|---|---|---|
| | M1 | M2 | F1 | F2 | M1 | M2 | F1 | F2 |
| Heart | 51.59 | 44.11 | 46.31 | 38.79 | 0.12 | 0.85 | 1.57 | 0.02 |
| Quadricep | 21.63 | | | | 0 | 0.74 | 0 | 0 |
| Diaphragm | 20.55 | 19.89 | 19.29 | 22.63 | 0 | 0.7 | 0.42 | 0.02 |
| Spinal Cord | 9.05 | 3.5 | 7.17 | 7.78 | 0 | 0.77 | 0.96 | 0.05 |

*Cells without data were due to experimental error.

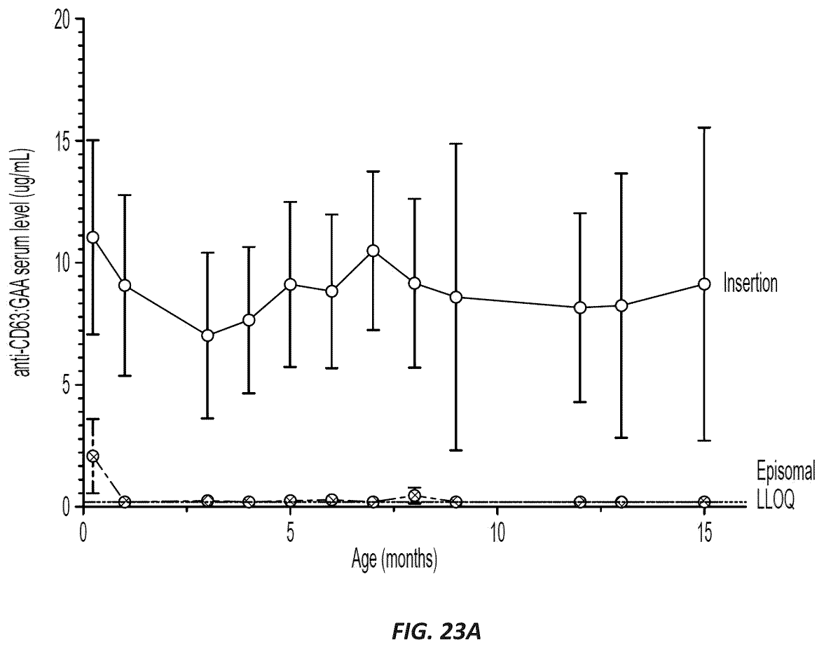

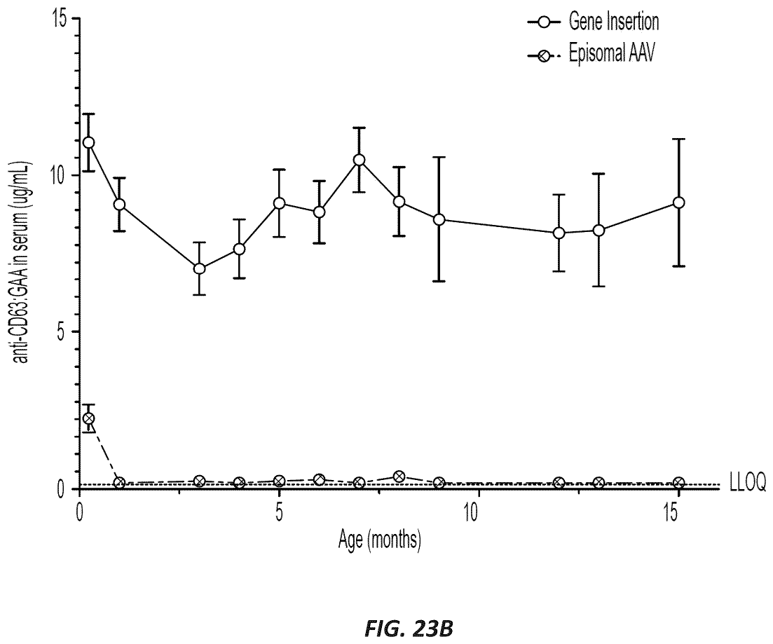

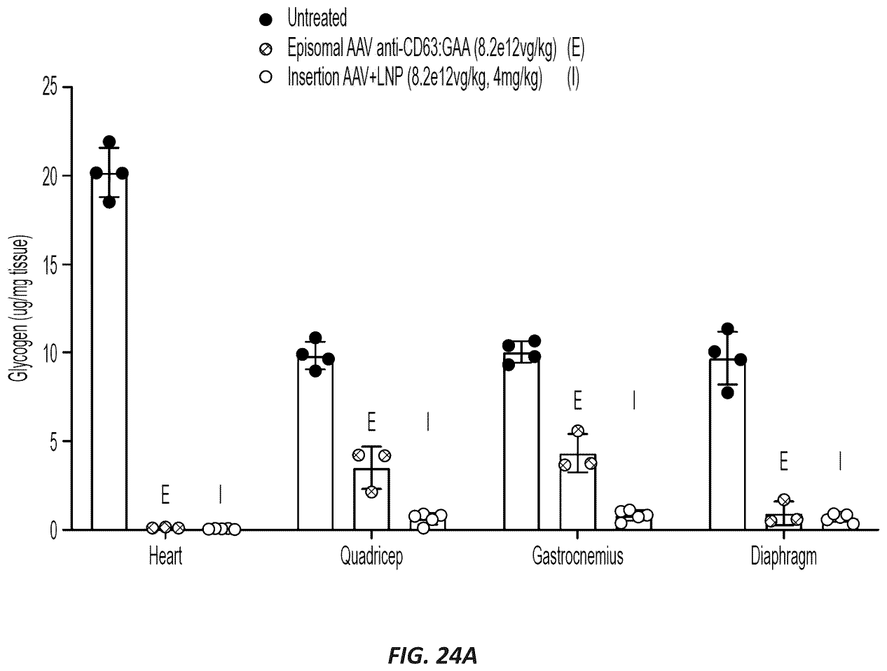

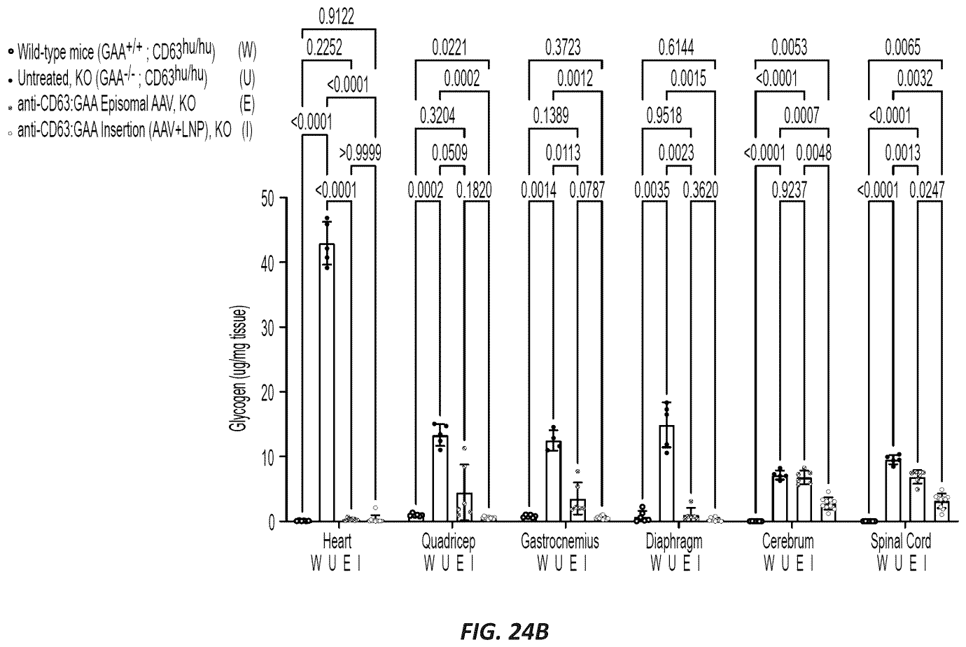

Similar experiments were then performed in which neonatal Gaa^{-/-};Cd63^{hu/hu} mice (62.5% C57BL/6, 37.5% 129Sv) were dosed intravenously at P1 with the following: (1) 8.2e12 vg/kg recombinant AAV8 encoding anti-CD63:GAA (REGV042); or (2) 4 mg/kg LNP-g666, and 8.2e12 vg/kg recombinant AAV8 anti-CD63:GAA insertion template (REGV044). Untreated Gaa^{-/-};Cd63^{hu/hu} mice and wild type mice were used as controls. Blood was collected and serum prepared at 7 days, 30 days, 2 months, 3 months, 4 months, 5 months, 6 months, 7 months, 8 months, 9 months, 12 months, 13 months, and 15 months, and tissues were collected at 3 months and 15 months post-administration. As shown in FIGS. 23A-23B and Tables 52-53, in contrast to what was observed when adult mice were dosed, the serum anti-CD63:GAA levels were stable over the 15-month time course in the insertion group, but the episomal group started out lower and dropped off to below the lower limit of quantification using the serum ELISA assay within 1 month when neonatal mice were dosed. Similarly, as shown in FIG. 24A and Table 54, glycogen storage at 3 months was normalized to wild type levels in heart, quadricep, gastrocnemius, and diaphragm in the insertion group, but not in the episomal group. Likewise, as shown in FIG. 24B and Table 55, glycogen storage at 15 months was normalized to wild type levels in heart, quadricep, gastrocnemius, and diaphragm in the insertion group, and glycogen storage was partially corrected in CNS tissues in the insertion group but not the episomal group.

TABLE 52
Serum Anti-CD63:GAA Levels (μg/mL) in Neonatal Mice with Insertion Group.

| | Insertion (AAV + LNP) | | | | | | | | | | | | | | | | | | |
|---|---|---|---|---|---|---|---|---|---|---|---|---|---|---|---|---|---|---|---|
| Months | * | * | ** | ** | ** | | | | | | | | ** | | | | * | * | * |
| 0.23 | 18.3 | 14.4 | 15.9 | 12.2 | 12.8 | 8.7 | 5.9 | 15.1 | 9.5 | 14.6 | 8.2 | 11.6 | 5.7 | 8.4 | 14 | 13.7 | 8.8 | 8.6 | 3.4 |
| 1 | 13.2 | 14.8 | 13.9 | 9 | 10.3 | 5.4 | 3.9 | 11.1 | 6.1 | 11.2 | 8 | 9.9 | 4.7 | 9.4 | 11.5 | 8.7 | 10.2 | 11.1 | 0 |
| 3 | 10.2 | 10.9 | | | | 3.5 | 2.3 | 10.3 | 5.8 | 7.2 | 6.9 | 8.2 | 2.7 | 7.9 | 14.7 | 5.5 | 7.2 | 6.2 | 2.8 |
| 4 | | | | | | 5.2 | 2.8 | 8.7 | 7.2 | 8.7 | 6.8 | 8.4 | | 7.4 | 14.5 | 6.8 | | | |
| 5 | | | | | | 7.1 | 4.1 | 12.1 | 6.6 | 10.2 | 8.1 | 9 | | 8.5 | 16.6 | 8.8 | | | |
| 6 | | | | | | 6.9 | 4.2 | 14.8 | 7 | 10.2 | 7 | 7.9 | | 9.2 | 13.1 | 8 | | | |
| 7 | | | | | | 8.9 | 4.9 | 17.5 | 11.2 | 10.4 | 9.9 | 10 | | 9.1 | 13.4 | 9.6 | | | |
| 8 | | | | | | 8.5 | 4.4 | 17 | 6 | 8.9 | 8.4 | 8.5 | | 8.1 | 12.5 | 9.3 | | | |
| 9 | | | | | | 5.9 | 3.1 | 10.7 | 5.2 | 25.4 | 7.5 | 5.9 | | 6.5 | 9.4 | 6.2 | | | |
| 12 | | | | | | 6.7 | 3.6 | 10.3 | 6.8 | 17.8 | 6.8 | 7.3 | | 6.7 | 9.7 | 5.9 | | | |
| 13 | | | | | | 4.6 | 3.7 | 17.4 | 4.2 | 17.0 | N/A | 6.1 | | 5.3 | 10.3 | 5.6 | | | |
| 15 | | | | | | 5.1 | 3.8 | 18.4 | 4.0 | 18.3 | 7.6 | 3.9 | | 5.5 | 18.0 | 6.6 | | | |

* Mouse sacrificed at 3 months for 3-month glycogen assay
** Mouse died

TABLE 53
Serum Anti-CD63:GAA Levels (μg/mL)
in Neonatal Mice with Episomal Group.

| | Episomal AAV | | | | | | | | | | |
|---|---|---|---|---|---|---|---|---|---|---|---|
| Months | * | * | * | ** | | | ** | | | | |
| 0.23 | 0 | 0 | 1.9 | 0 | 2.2 | 4.1 | 2.5 | 2.7 | 3.7 | 3.4 | 3.6 |
| 1 | 0 | 0 | 0 | 0 | 0 | 0 | 0 | 0 | 0 | 0 | 0 |
| 3 | 0 | 0 | 0 | 0 | 0 | 0 | 0 | 0.3 | 0.4 | 0.5 | 0 |
| 4 | | | | 0 | 0 | 0 | 0 | 0 | 0 | 0 | 0 |
| 5 | | | | | 0 | 0 | 0 | 0 | 0 | 0.6 | 0 |
| 6 | | | | | 0 | 0 | 0 | 0 | 0.5 | 0.6 | 0 |
| 7 | | | | | 0 | 0 | 0 | 0 | 0 | 0 | 0 |
| 8 | | | | | 0 | 0.5 | 0 | 0 | 0.5 | 1 | 0 |
| 9 | | | | | 0 | 0 | 0 | 0 | 0 | 0 | 0 |
| 12 | | | | | 0 | 0 | 0 | 0 | 0 | 0 | 0 |
| 13 | | | | | 0 | 0 | 0 | 0 | 0 | 0 | 0 |
| 15 | | | | | 0 | 0 | | 0 | 0 | 0 | 0 |

* Mouse sacrificed

TABLE 54
Glycogen Levels (μg/mg Tissue) in Neonatal Mice.

| | Untreated KO | | | | Episomal AAV | | | Gene Insertion | | | | |
|---|---|---|---|---|---|---|---|---|---|---|---|---|
| Heart | 20.16 | 18.52 | 20.14 | 21.92 | 0.16 | 0.1 | 0.1 | 0.08 | 0.06 | 0.08 | 0.04 | 0.02 |
| Quadricep | 8.96 | 10.83 | 9.89 | 9.63 | 4.18 | 2.13 | 4.21 | 0.89 | 0.77 | 0.82 | 0.1 | 0.58 |
| Gastrocnemius | 10.39 | 10.65 | 9.31 | 9.77 | 5.58 | 3.66 | 3.75 | 1.04 | 0.83 | 1.12 | 0.38 | 0.73 |
| Diaphragm | 10.05 | 7.73 | 11.33 | 9.6 | 0.5 | 0.59 | 1.7 | 0.57 | 0.7 | 0.85 | 0.34 | 0.9 |

TABLE 55
Glycogen Levels (μg/mg Tissue) in Neonatal Mice.

| | WT | | | | | | | | | |
|---|---|---|---|---|---|---|---|---|---|---|
| Heart | 0.18 | 0.03 | 0.09 | 0.12 | 0.04 | 0.04 | | | | |
| Quadricep | 0.95 | 0.77 | 0.86 | 1.01 | 1.35 | 0.85 | | | | |
| Gastrocnemius | 0.8 | 0.68 | 0.61 | 1.02 | 1.06 | 0.53 | | | | |
| Diaphragm | 0.6 | 0.12 | 2.24 | 1.25 | 0.22 | 0.03 | | | | |
| Cerebrum | 0.02664 | 0 | 0 | 0.04436 | 0 | 0.00255 | | | | |
| Spinal Cord | 0.01999 | 0 | 0 | 0 | 0.026 | 0 | | | | |
| | Untreated | | | | | | | | | |
| Heart | 40.73 | 42.15 | 45.87 | 46.84 | 39.15 | | | | | |
| Quadricep | 12.42 | 11.02 | 14.71 | 15.05 | 13.43 | | | | | |
| Gastrocnemius | * | 10.99 | 11.6 | 14.56 | 12.86 | | | | | |
| Diaphragm | 16.49 | 10.57 | 18.33 | 17.3 | 11.87 | | | | | |
| Cerebrum | 7.28 | 6.26 | 7.02 | 6.98 | 8.23 | | | | | |
| Spinal Cord | 8.46 | 10.34 | 9.37 | 9.94 | 9.49 | | | | | |
| | P1 episomal | | | | | | | | | |
| Heart | 0.42 | 0.17 | 0.69 | 0.21 | 0.09 | 0.09 | | | | |
| Quadricep | 11.27 | 8.47 | 1.85 | 2.72 | 1.43 | 0.96 | | | | |
| Gastrocnemius | 7.73 | 5.36 | 2.02 | 2.18 | 2.03 | 1.84 | | | | |
| Diaphragm | 3.09 | 0.8 | 0.74 | 0.76 | 0.33 | 0.42 | | | | |
| Cerebrum | 8.26 | 5.63 | 7.25 | 7.48 | 5.87 | 6.33 | | | | |
| Spinal Cord | 6.42 | 4.93 | 7.46 | 7.13 | 7.61 | 7.55 | | | | |
| | P1 insertion | | | | | | | | | |
| Heart | 0.04 | 0.12 | 0.13 | 0.06 | 0.25 | 2.1 | 0 | 0.05 | 0.04 | 0.07 |
| Quadricep | 0.6 | 0.64 | 0.68 | 0.54 | 0.58 | 0.61 | 0.27 | 0.85 | 0.35 | 0.5 |
| Gastrocnemius | 0.43 | 0.65 | 0.51 | 0.51 | 0.46 | 0.9 | 0.27 | 1.06 | 0.24 | 0.43 |
| Diaphragm | 0.16 | 0.29 | 0.08 | 0.12 | 0.39 | 0.77 | 0 | 0 | 0.59 | 0.12 |
| Cerebrum | 3.09 | 4.59 | 1.18 | 3.06 | 1.87 | 2.42 | 3.33 | 2.2 | 2.39 | 3.29 |
| Spinal Cord | 3.61 | 4.89 | 1.88 | 3.55 | 1.03 | 3.79 | 3.39 | 3.72 | 2.23 | 3.42 |

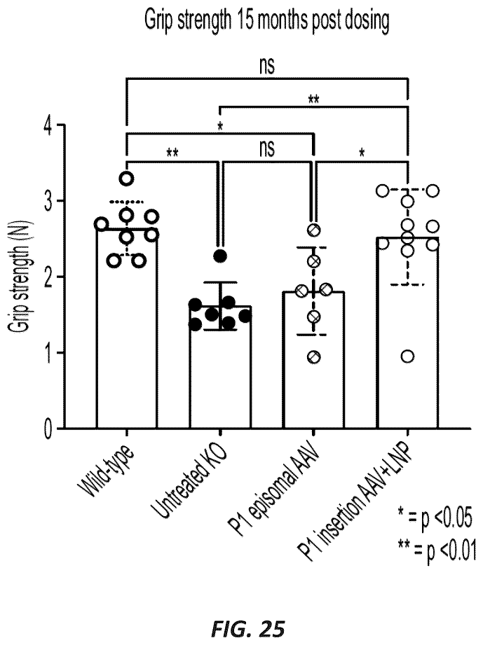

To assess whether the improved glycogen reduction observed with the insertion template in neonatal mice translated into improved muscle function, the mice were tested on grip strength apparatuses at 15 months post-administration. Limb grip strength was measured with a force meter (Columbus Instruments, Columbus, OH, USA). All tests were performed in triplicate. Mice treated with the insertion template showed significantly improved performance compared to mice treated with the episomal construct on the grip strength test. In fact, the grip strength in the insertion group tracked closely with that of wild type mice at 15 months post-treatment, whereas there was no difference in the grip strength in the episomal group tracked compared to the untreated group. See FIG. 25 and Table 56. These results show that, in neonatal mice, the insertion approach shows vastly improved durability of expression compared to the episomal approach, and better substrate reduction, indicating that insertion is the superior approach for pediatric indications.

TABLE 56
Grip Strength (Newtons) in Neonatal Mice.

| Wild-type | Untreated KO | P1 insertion AAV + LNP | P1 episomal AAV |
|---|---|---|---|
| 2.21 | 1.39 | 2.44 | 1.83 |
| 2.21 | 1.63 | 2.34 | 1.47 |
| 2.52 | 1.48 | 2.51 | 0.94 |
| 2.55 | 1.66 | 2.68 | 1.81 |
| 2.81 | 1.37 | 3.13 | 2.2 |
| 2.69 | 2.27 | 0.95 | 2.61 |
| 3.29 | 1.5 | 2.99 | |
| 2.79 | | 2.42 | |
| | | 2.66 | |
| | | 3.13 | |

An experiment was performed in which 4-month old Gaa^{-/-};Alb^{hu/hu} mice (n=3) were dosed intravenously with 7.5e10 vg/mouse recombinant AAV8 anti-CD63:GAA insertion template (REGV044) and 1 mg/kg LNP-g9860 in order to validate that anti-CD63:GAA can be inserted into mice humanized for albumin using human albumin gRNA. Blood was collected and serum prepared at 7 days, 14 days, 35 days, and 60 days post-administration. GAA serum levels up to ˜3 μg/mL were observed and were maintained over the time course (data not shown), confirming that anti-CD63:GAA can be inserted into mice humanized for albumin using human albumin gRNA.

In summary, the combination of the highly precise and targeted CRISPR/Cas9 technology delivered by LNP and the anti-CD63:GAA DNA template delivered by the selected rAAV8 vector allows for long-term expression of anti-CD63:GAA protein from hepatocytes and delivery to muscle cells affected in PD, potentially providing a life-long effective treatment to PD patients, including neonatal patients.

These results show that, in neonatal mice, the insertion approach shows vastly improved durability of expression compared to the episomal approach, indicating that insertion is the superior approach in neonatal subjects.

### Example 13. Optimized Anti-CD63:GAA DNA Templates

Optimized anti-CD63:GAA templates were generated to develop a lead for non-human primate (NHP) studies. To select a development cantidate, several versions of the insertion cassette were generated in which the nucleotide sequence encoding the anti-CD63:GAA was modified (e.g., by depleting CpGs). Table 57 lists the different versions of anti-CD63:GAA inserts designed. In addition, the GS 50 construct was further optimized to remove the six activating CpG motifs but leaving the 44 non-activating CpG motifs (GS 44; SEQ ID NO: 740). The GS 0 construct was further optimized (GS 0v2; SEQ ID NO: 738) by removing an AATAA polyA, removing a cryptic splice acceptor, and replacing a glycine encoding GGGGGGGGGGG (SEQ ID NO: 743) with GGAGGAGGTGG (SEQ ID NO: 744).

TABLE 57
Anti-CD63:GAA Inserts for Insertion Cassettes.

| Anti-CD63:GAA Insert | Transgene CpG Content | SEQ ID NO |
|---|---|---|
| First generation | 181 | 734 |
| GS 0 | 0 | 737 |
| GS 0v2 | 0 | 738 |
| DC 0 | 0 | 735 |
| GS 1 | 1 | 739 |
| RE 8 | 8 | 742 |
| GS 50 | 50 | 741 |
| GS 44 | 44 | 740 |
| GA 0 | 0 | 736 |

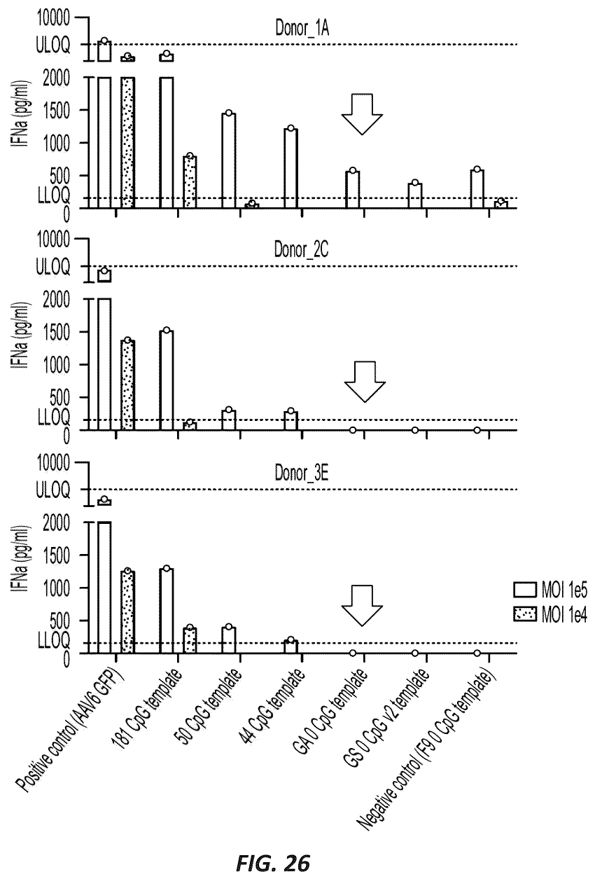

Peripheral blood mononuclear cells (PBMCs) were isolated from human blood. Plasmacytoid dendritic cells (pDCs) were enriched and combined with pBMCs (1e4 pDCs+1e5 PBMCs per well). The cells were incubated for 16-18 hours with AAV6 or control CpG-oligodeoxynucleotides (ODNs). The supernatants were harvested, and an IFNα ELISA was performed. This assay assessed whether CpG-depleted anti-CD63:GAA sequences exhibited reduced IFN—I responses in a primary human plasmacytoid DC-based assay as compared to non-CpG-depleted sequences. The tested samples, including a positive control AAV6-GFP and a negative control CpG-depleted (0 CpG F9 transgene) template, are shown in Table 58. The results are shown in FIG. 26.

TABLE 58
AAV6 Anti-CD63:GAA Templates Tested
for IFNα response in pDC/AAV assay.

| | Transgene CpG | TOTAL CpG |
|---|---|---|
| Genome | content | content w/ITRs |
| AAV6 GFP | 99 | 175 |
| (SFFV_eGFP_WPRE) | | |
| First generation | 181 | 213 |
| GS 50 | 50 | 82 |
| GS 44 | 44 | 75 |
| GA 0 | 0 | 32 |
| GS 0 v2 | 0 | 32 |
| F9 0 CpG template | 0 | 33 |

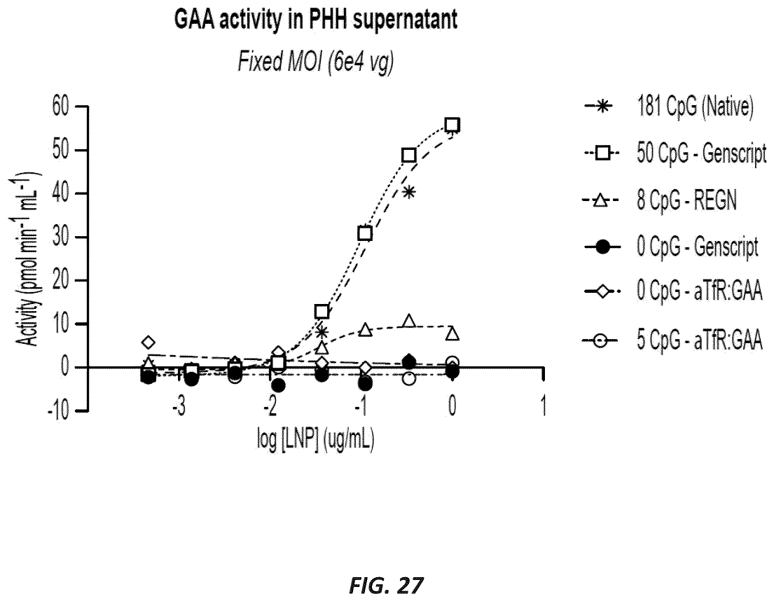

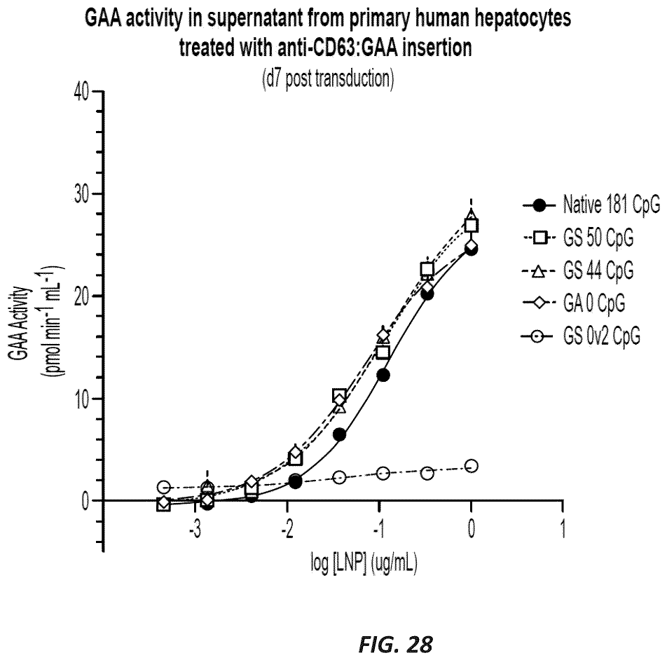

Activity of the optimized templates was tested in a primary human hepatocyte assay. AAV templates were packaged into AAV2 viruses. Primary human hepatocytes were grown in 96-well plates and administered the AAV (AAV2, AAV6, or AAV8) containing the template DNA and LNP-g9860 at either fixed MOI with LNP dose titration or fixed LNP concentration with AAV dose titration. Supernatants were collected 7 days post-dosing and stored at −80 degrees Celsius. Supernatants were thawed and GAA activity in the supernatants was measured using a 4-methylumbelliferone-based fluorometric assay (K690, BioVision, Milpitas, CA, USA) as a measurement of amount of enzymatically active GAA produced and secreted from the cells. A first batch of optimized anti-CD63:GAA and anti-TfR:GAA templates were tested using AAV2 as shown in Table 59 and FIG. 27. The 50 CpG anti-CD63:GAA template expressed similarly to the native template, but the initial templates with fewer CpGs did not express well. Additional optimized anti-CD63:GAA templates were tested using AAV6 as shown in Table 60 and FIG. 28. The GA 0 CpG anti-CD63:GAA template maintained expression compared to the native high CpG template in the in vitro primary human hepatocyte insertion assay. Similar results were observed using AAV8 instead of AAV6 (data not shown). The GA 0 CpG anti-CD63:GAA template produced functional GAA in the primary human hepatocyte assay at levels similar to the native while also producing reduced IFNα responses in the pDC/AAV assay.

TABLE 59
AAV2 Anti-CD63:GAA and Anti-TfR:GAA Templates
Tested for GAA Activity in PHH Supernatant.

| Genome | Transgene CpG content |
|---|---|
| First generation | 181 |
| GS 50 | 50 |
| RE 8 | 8 |
| GS 0 | 0 |
| 12837scfv-GAA-1 0 CpG 27 | 0 |
| 12837scfv-GAA-2 5 CpG 27 | 5 |

TABLE 60
AAV6 Anti-CD63:GAA Templates Tested
for GAA Activity in PHH Supernatant.

| Genome | Transgene CpG content |
|---|---|
| First generation | 181 |
| GS 50 | 50 |
| GS 44 | 8 |
| GA 0 | 0 |
| GS 0v2 | 0 |

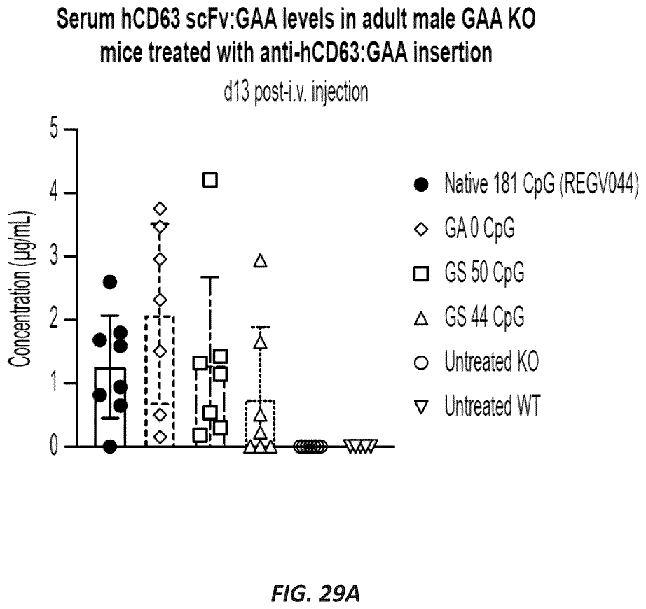

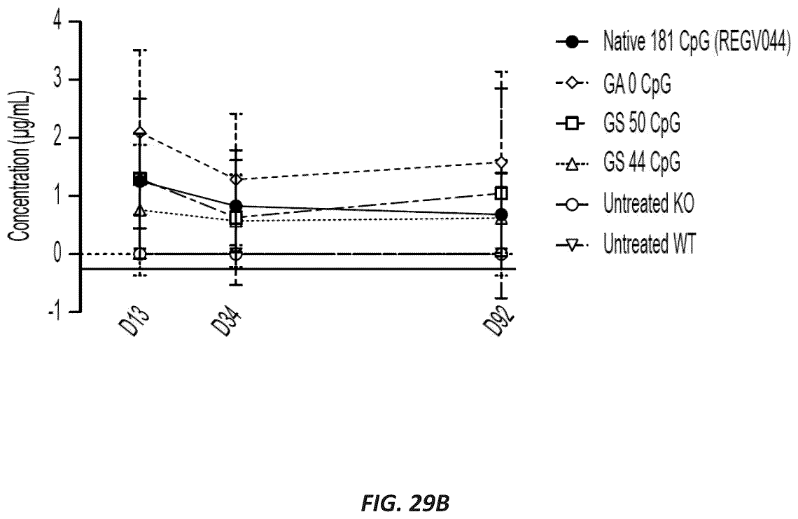

Expression of the optimized templates was validated following insertion in Gaa^{-/-} mice as described in Example 12. Adult mice were dosed intravenously at with 1.97e12 vg/kg recombinant AAV8 anti-CD63:GAA insertion template (REGV044) and 1 mg/kg LNP-g666. The recombinant AAV8 templates tested included the native 181 CpG anti-CD63:GAA insertion template (REGV044), the GA 0 CpG template, the GSa50 CpG template, and the GS 44 CpG template. Untreated Gaa^{-/-} mice and wild type mice were used as controls. Blood was collected and serum prepared at 13 days, 34 days, and 92 days post-administration. As shown in FIG. 29A, the GA 0 CpG template showed similar serum expression in vivo in adult Gaa^{-/-} mice compared to the native high CpG template. As shown in FIG. 29B, the serum expression levels were consistent over three time points.

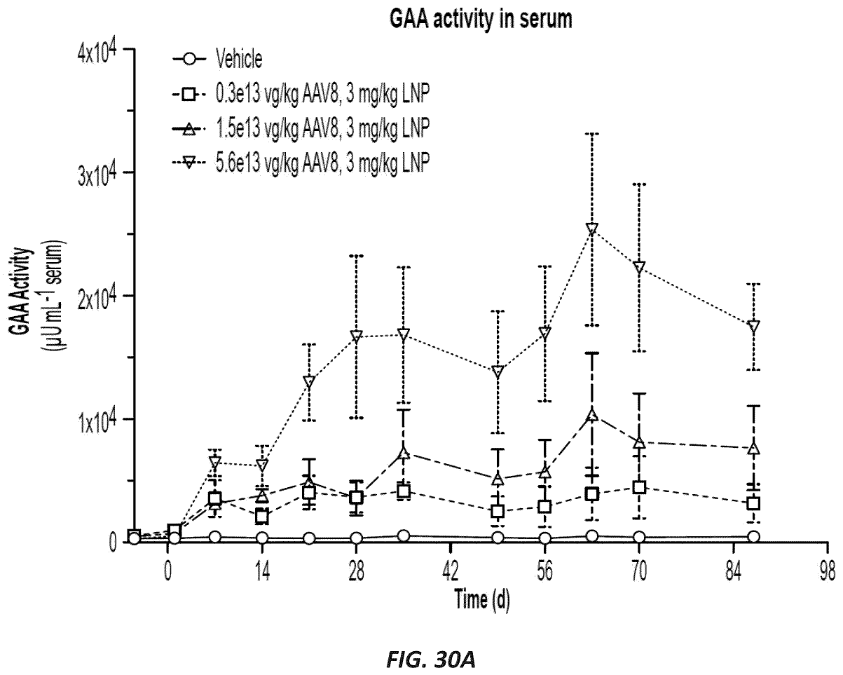

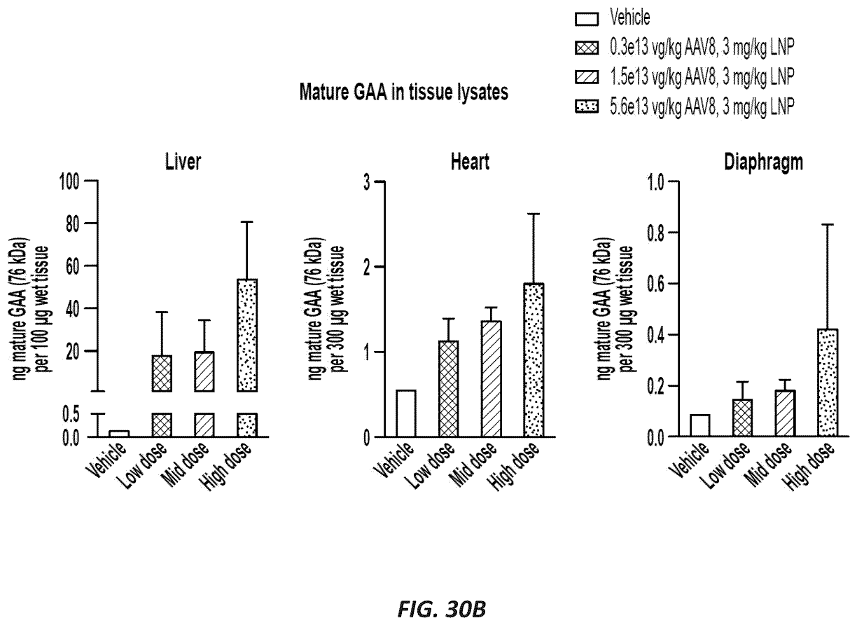

Expression of the GA 0 CpG anti-CD63:GAA template (SEQ ID NOS: 650 and 679) was evaluated following insertion in non-human primates. Two-year old cynomolgus macaques were administered recombinant AAV8 containing the CpG depleted anti-CD63:GAA template and an LNP-g9860 targeting the cynomolgus albumin intron 1. Three different recombinant AAV8 doses were used (0.3e13 vg/kg, 1.5e13 vg/kg, and 5.6e13 vg/kg) with a 3 mg/kg LNP dose. N=1 in the vehicle control group, and N=3 in the dosed groups. Serum GAA activity was measured using a fluorometric substrate assay in the monkeys through the course of the study. An AAV-dose-dependent increase in serum GAA activity levels was observed over time in the groups (FIG. 30A). This indicated successful insertion of the anti-CD63:GAA transgene into the albumin locus and resultant expression and secretion of the transgene in the animals. Tissues were collected at sacrifice (Day 89) and probed by western blot for presence of a 76 kDa lysosomal form of GAA. A dose-dependent increase of lysosomal GAA in the tissues was observed in the heart and diaphragm (FIG. 30B), which indicated delivery of the liver-derived anti-CD63:GAA protein to the distal tissues.

Activity of the optimized templates is validated in the PD mouse model, Gaa^{-/-};Cd63^{hu/hu}, as described in Example 4.

Expression of the optimized templates is evaluated in non-human primates. Two LNP doses and two gRNAs are tested. Specifically, expression is evaluated by administering 1 or 3 mg/kg of LNP-g9860 as described in Example 1 and 1.5e13 vg/kg of rAAV8 comprising each optimized template. An LNP with gRNA9844 is also used. Expression is analyzed over a 12-week study. Tissues are also collected for analysis of biodistribution of GAA, and GAA activity is assessed in collected tissues.

### Example 14. Optimized Anti-CD63:GAA and Anti-TfR:GAA DNA Templates to Minimize Mis-Splicing

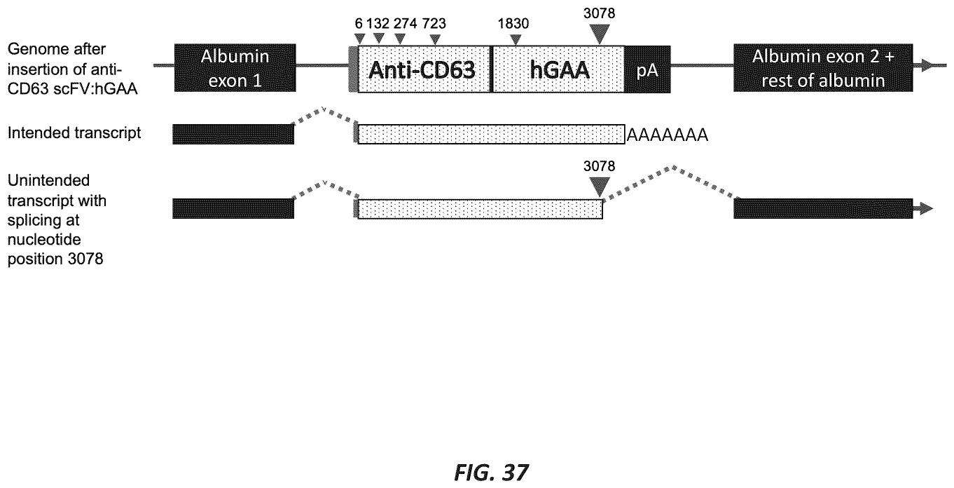

To take advantage of the endogenous promoter and other regulatory elements in the albumin locus, the anti-CD63:GAA and anti-TfR:GAA DNA templates described herein do not contain a promoter itself but instead contain a splice acceptor. This adds a margin of safety, because if the DNA template were to randomly integrate or integrate in an off-target manner, there is no promoter present to potentially affect the regulation and expression of neighboring genes. In the system described herein, the guide RNA targets Cas9 to cut at a site in albumin intron 1, thus directing insertion of the DNA template to that site via non-homologous end joining. When transcription occurs at an albumin locus containing a gene insertion, transcription proceeds through albumin 5′ UTR, exon 1, the 5′ part of intron 1, the insertion sequence, and through a polyadenylation (polyA) sequence that is included at the 3′ end of the DNA insertion template. The RNA polymerase will then continue to transcribe the next several hundred to few thousand bases while the RNA cleavage and polyadenylation machinery acts on the polyA site. If this next several hundred to few thousand bases contains a splice acceptor site, it is possible that splicing can occur to this downstream splice acceptor site, cutting out the polyA. If splicing occurs from the splice donor at the end of albumin exon 1 to the 5′ of albumin exon 2, this creates a normal albumin transcript. However, if there are cryptic splice donors in the inserted DNA template, some portion of the transcript may be mis-spliced from a cryptic splice donor in the middle of the transcript to a downstream splice acceptor (such as the one at the 5′ end of albumin exon 2). See FIG. 37. Indeed, we found these mis-spliced events in a subset of transcripts when we sequenced liver lysates or cultured hepatocytes containing gene insertions of first generation, codon-optimized and CpG depleted anti-CD63:GAA or anti-TfR:GAA templates. For example, cryptic splice donors introduced by codon optimization and CpG depletion combined with weak transcript termination allowed mis-splicing into albumin exon 2. As shown in the intended transcript in FIG. 37, the nascent transcript should be cleaved and polyadenylated the polyadenylation sequence (pA). However, transcription may continue into ALB exon 2, and splicing machinery may get to the transcript before transcript cleavage/polyadenylation and splice from a cryptic splice donor to the ALB exon 2 splice acceptor as shown with the unintended transcript.

Sequences of these mis-spliced transcripts revealed several cryptic splice donors (i.e., sequences that function as splice donors in some fraction of transcripts but do not have a strong splice donor consensus sequence) within the sequence when the lead codon-optimized anti-CD63:GAA insertion template (coding sequence set forth in SEQ ID NO: 650 and construct set forth in SEQ ID NO: 783; construct VVT1254) or lead anti-TfR:GAA insertion template (coding sequence set forth in SEQ ID NO: 669 and construct set forth in SEQ ID NO: 779; construct VVT874) was inserted into the albumin locus.

The splicing pattern of ALB-anti-CD63:GAA fusion transcripts was evaluated by short-read RNA sequencing of liver samples from cynomolgus monkeys following administration of construct VVT1254 and LNP-g9860. Sequencing reads from all samples in the experiment were combined for analysis, and nucleotide positions with unintended transcripts at percentages equal to or greater than 1.0% were identified (FIG. 37, Table 61 (the GT or GC splice donor motifs and CAG splice acceptor motif in the nucleotide sequence are underlined)). The unintended transcripts identified were formed by splicing from cryptic splice donors in anti-CD63:GAA at nucleotide positions 132, 274, 723, 1830, or 3078. Position numbering is based on the last nucleotide present in the transgene before splicing out, where position 1 is the first nucleotide following the mouse Alb exon 2 splice acceptor in the anti-CD63:GAA nucleic acid constructs. The majority of these unintended transcripts were spliced from cryptic donor sites into the native splice acceptor preceding ALB exon 2. The unintended transcripts identified were also formed by splicing from the native splice donor following ALB exon 1 into the cryptic splice acceptor in anti-CD63: GAA at nucleotide position 6.

TABLE 61
Cryptic Splice Donors and Acceptors Identified
in ALB-anti-CD63:GAA Fusion Transcripts in
Cynomolgus Monkeys.

| | | | | % |
|---|---|---|---|---|
| | | | | Unintended |
| Nucleotide | | | | Transcripts |
| Position | | Cryptic | Location | at Position |
| in anti- | Splice | Site | in | in Cynomolgus |
| CD63:GAA | Type | Sequence | Transgene | Monkeys |
| 6 | Acceptor | CTTCAGGTC | anti-CD63 | 1.1% |
| | | | scFv | |
| 132 | Donor | AAAGGCCTG | anti-CD63 | 2.4% |
| | | | scFv | |
| 274 | Donor | CAGTGTACT | anti-CD63 | 1.8% |
| | | | scFv | |
| 723 | Donor | AAAGGTGGT | anti-CD63 | 1.8% |
| | | | scFv | |
| 1830 | Donor | CAAGGTGGT | hGAA | 1.1% |
| 3078 | Donor | AAAGGTGGA | hGAA | 13.8% |

Consistent with this, high molecular weight bands were observed in an anti-GAA western blot on NHP liver lysates from lead anti-CD63:GAA template-treated NHPs that were not observed in studies with the native, non-codon-optimized insertion template (data not shown).

The splicing pattern of ALB-anti-TfR:GAA fusion transcripts was evaluated by short-read RNA sequencing of primary human hepatocytes (PHH) from xenografted mouse livers (Phoenix Bio) following administration of construct VVT874 and LNP-g9860. Nucleotide positions with unintended transcripts at percentages equal to or greater than 1.0% were identified (Table 62 (the GT or GC splice donor motifs in the nucleotide sequence are underlined)). The unintended transcripts identified were formed by splicing from cryptic splice donors in anti-TfR:GAA at nucleotide positions 1857, 2331, or 3105. Position numbering is based on the last nucleotide present in the transgene before splicing out, where position 1 is the first nucleotide following the mouse Alb exon 2 splice acceptor in the anti-CD63:GAA nucleic acid constructs. The majority of these unintended transcripts were spliced from cryptic donor sites into the native splice acceptor preceding ALB exon 2. The cryptic splice donors in anti-TfR:GAA at nucleotide positions 1857 and 3105 correspond to the identified cryptic splice donors in anti-CD63:GAA at nucleotide positions 1830 and 3078.

TABLE 62
Cryptic Splice Donors Identified in ALB-anti-
TfR: GAA Fusion Transcripts in Primary Human
Hepatocytes.

| Nu- | Cor- | | | | |
|---|---|---|---|---|---|
| cleo- | respond- | | | | % |
| tide | ing | | | | Unin- |
| Posi- | Nucleo- | | | | tended |
| tion | tide | | | Loca- | Trans- |
| in | Position | | | tion | cripts |
| anti- | in anti- | | Cryptic | in | at Po- |
| TfR: | CD63: | Splice | Site | Trans- | sition |
| GAA | GAA | Type | Sequence | gene | in PHH |
| 1857 | 1830 | Donor | CAAGGTGGT | hGAA | 1.0% |
| 2331 | 2304 | Donor | AGAGGCACC | hGAA | 1.0% |
| 3105 | 3078 | Donor | AAAGGTGGA | hGAA | 37.5% |

To minimize mis-splicing, we applied a two-pronged approach: (1) identifying cryptic splice donors functionally via RNA sequencing (RNA-Seq), rather than prediction based on consensus sequences, and introducing synonymous mutations that disrupt the critical “GU” nucleotide pair that forms the core of the splice donor sequence, and (2) adding additional elements which have the net effect of increasing the time from when the polyA is transcribed to when RNA polymerase reaches the next splice acceptor site. More details on these approaches individually are below; we have also used the approaches in combination and found an additive effect.

Approach 1: While we screened for consensus splice donor sequences during the codon optimization and CpG removal process in Examples 10 and 13, when we examined transcripts by RNA-Seq we surprisingly found that a significant percentage of total transcripts (20-30%) were mis-spliced from a cryptic splice donor site in the cDNA for anti-CD63:GAA and anti-TfR:GAA, depending on the cell type tested. By RNA-Seq, we were able to determine the critical GU pair that facilitated mis-splicing at these sites and introduce synonymous mutations that eliminated mis-splicing from those positions.

Approach 2: We introduced either concatenated polyA signals (bovine growth hormone (BGH) and SV40), MAZ elements (SEQ ID NO: 755) which cause polymerase pausing, or additional stuffer sequence (SEQ ID NO: 754) to increase the time between when RNA polymerase transcribes the polyA to the time when it transcribes the next splice acceptor. We also tested the BGH polyA (SEQ ID NO: 751) and synthetic polyA (SPA) (SEQ ID NO: 753) sequences individually. The SV40 polyA is bidirectional, but the polyadenylation in the “late” orientation is more efficient than the polyadenylation in the “early” orientation. The SV40 polyA element is in the “early” orientation in the lead codon-optimized anti-CD63:GAA and anti-TfR:GAA templates. This orientation allows transcription to proceed through polyA, allowing cryptic splice donor sites within transgene to be utilized. This might be corrected by inverting the pA to the “late” orientation, using BGHpA, adding additional tandem polyAs, etc. There is a weak splice donor motif at position 3078 that is consistently detected in the RNA-Seq data as contributing to the largest fraction of observed unintended splicing products. This site can be mutated. Notably, to concatenate SV40 “Late” polyA with BGH, we mutated the transcription terminator sequences that are present in the “early” inverse orientation of SV40, thus making this version of the SV40 polyA unidirectional rather than bidirectional (“Sv40LuniPA” (SEQ ID NO: 752)). Thus, if our DNA insertion template is inserted into the genome in the non-functional “reverse” orientation, transcription should proceed straight through the entire albumin locus and the non-functional insertion should be spliced out along with the first intron, as there are no transcription terminator sequences present in the “reverse” orientation. The particular polyA sequences used were chosen as they contained no CpG dinucleotides.

As shown below, by combining both approaches, we were able to reduce the number of unintended, mis-spliced transcripts from >20% to <1% of total transcripts. This may improve protein expression, reduce potentially immunogenic fusion peptides from forming, and reduce ER stress and the unfolded protein response in the cells producing the protein product. In addition, as shown below, no loss of GAA activity was observed from any splice site or polyA modification. The constructs tested are shown in Table 63.

TABLE 63
Anti-CD63:GAA and Anti-TfR:GAA Templates Modified to Reduce Cryptic Splicing.

| Template | Comments | Status |
|---|---|---|
| VVT1125 - pAAV- | 1830 & 3078 GAA cryptic splice site corrections | Top performer in |
| TfR1GAA(GS0v2)-2Xfix- | Original PolyA replaced w/tandem bGH + SV40-late | PHH for GAA |
| bGH.sv40LuniPA (SEQ ID NO: | pA w/reverse strand AAUAAAs mutated | activity assay |
| 764) | | |
| VVT1126 - pAAV- | Original PolyA replaced w/bGHpA | |
| TfR1GAA(GS0v2)-bGHpA swap | | |
| (SEQ ID NO: 766) | | |
| VVT1129 - pAAV- | 1830 & 3078 GAA cryptic splice site corrections | Top performer in |
| TfR1GAA(GS0v2)-2Xfix-bGHpA | Original SV40-early PolyA replaced with bGHpA | PHH for GAA |
| (SEQ ID NO: 765) | | activity assay |
| VVT1118 - pAAV-CD63GAA- | Mutate position 3078 & insert bGHpA | |
| 3078fix-bGHpA (SEQ ID NO: 771) | | |
| VVT1119 - pAAV-CD63GAA- | Original PolyA replaced w/tandem bGHpA + SPA | |
| bGHpA.SPA swap (SEQ ID NO: | | |
| 773) | | |
| VVT1120 - pAAV-CD63GAA- | Original PolyA replaced w/tandem bGHpA + SPA w/ | |
| bGHpA.stuffer.SPA (SEQ ID NO: | intervening stuffer sequence | |
| 774) | | |
| VVT1121 - pAAV-CD63GAA- | CD63:GAA w/4 cryptic splice site corrections | |
| 4Xfix-bGH.sv40LuniPA (SEQ ID | (274, 723, 1830, 3078) | |
| NO: 768) | Original SV40-early pA replaced w/tandem bGH + | |
| | SV40-late pA w/reverse strand AAUAAAs mutated | |
| VVT1122 - pAAV-CD63GAA- | CD63:GAA w/4 cryptic splice site corrections | |
| 4Xfix-bGHpA (SEQ ID NO: 769) | Original SV40-early pA replaced w/bGHpA | |
| VVT1128 - pAAV-CD63GAA- | CD63:GAA w/6 cryptic splice site corrections | Top performer in |
| 6Xfix-bGH.sv40LuniPA (SEQ ID | Original SV40-early pA replaced w/tandem bGH + | PHH for GAA |
| NO: 777) | SV40-late pA w/reverse strand AAUAAAs mutated | activity assay |
| VVT1139 - pAAV-CD63GAA- | CD63:GAA w/6 cryptic splice site corrections | Top performer in |
| 6Xfix-bGHpA (SEQ ID NO: 778) | Original SV40-early pA replaced w/bGHpA | PHH for GAA |
| | | activity assay |
| VVT1123 - pAAV-CD63GAA- | 3078 cryptic splice site correction | |
| 3078fix-bGH.sv40LuniPA (1 CpG) | Original SV40-early pA replaced w/tandem bGH + | |
| (SEQ ID NO: 770) | SV40-late pA w/reverse strand AAUAAAs mutated | |
| VVT1124 - pAAV-CD63GAA- | Original PolyA replaced w/bGHpA | Worst performer |
| bGHpA swap (SEQ ID NO: 772) | | in PHH for GAA |
| | | activity assay |
| VVT1127 - pAAV-CD63GAA- | MAZ repeats added after original SV40-early polyA | |
| SV40E-MAZ (SEQ ID NO: 776) | to promote transcription termination | |
| VVT1138 - pAAV-CD63GAA- | Original PolyA replaced w/bGHpA followed by | |
| bGHpA-MAZ (SEQ ID NO: 775) | MAZ repeats | |

The splicing pattern of ALB-anti-CD63: GAA fusion transcripts was evaluated by short-read RNA sequencing of primary human hepatocytes (PHH) from xenografted mouse livers (Phoenix Bio) following administration of one of constructs VVT1119-VVT1127 or VVT1138-VVT1139 and LNP-g9860. Cryptic splicing at previously identified sites in anti-CD63:GAA (nucleotide positions 6, 132, 274, 723, 1830, and 3078) were evaluated, and an additional cryptic splice donor site at nucleotide position 2934 occurring in multiple samples was identified (Table 64). Mutating cryptic splice sequences eliminated cryptic splicing at those positions. Mutating cryptic splice sequences also resulted in shifting cryptic splicing activity to a previously minimal site (i.e., position 2934) in constructs VVT1122 and VVT1139, and the amount of cryptic splicing that removed was much larger than the amount shifted. Comparing construct VVT1119 to construct VVT1124, adding synthetic polyA sequence decreased cryptic splicing, and comparing construct VVT1120 to construct VVT1119, stuffer and synthetic polyA sequence further decreased cryptic splicing. Comparing construct VVT1138 to construct VVT1124, adding MAZ element decreased cryptic splicing. Comparing construct VVT1123 to construct VVT1118 and comparing construct VVT1121 to construct VVT1122, adding SV40Late in tandem with bGH decreased cryptic splicing compared to bGH alone.

TABLE 64
Percentages of Unintended Transcript in ALB-anti-CD63:GAA Fusion Transcripts Following Administration
of AAV Packaged with anti-CD63:GAA Redesigns and LNP-g9860 in Primary Human Hepatocytes.

| | # Cryptic | | | | | | | | | Est. | Est. |
|---|---|---|---|---|---|---|---|---|---|---|---|
| | Sites | PolyA | Nucleotide Position in anti-CD63:GAA | | | | | | | Intended | Unintended |
| Template | Removed | Sequence | 6 | 132 | 274 | 723 | 1830 | 2934 | 3078 | Transcript** | Transcripts** |
| VVT1254 | 0 | SV40Early | 1.1% | 2.4% | 1.8% | 1.8% | 1.1% | 0.1% | 13.8% | 77.9% | 22.1% |
| VVT1124 | 0 | bGH | 0.7% | 0.1% | 0.2% | 6.2% | 1.3% | 0.0% | 26.1% | 65.4% | 34.6% |
| VVT1119 | 0 | bGH + | 0.1% | 0.3% | 0.3% | 1.9% | 0.4% | 0.0% | 15.8% | 81.2% | 18.8% |
| | | synthetic | | | | | | | | | |
| VVT1120 | 0 | bGH + stuffer + | 0.3% | 0.1% | 0.2% | 3.1% | 1.3% | 0.3% | 3.7% | 91.0% | 9.0% |
| | | synthetic | | | | | | | | | |
| VVT1138 | 0 | bGH + MAZ | 0.7% | 0.4% | 0.2% | 1.9% | 0.6% | 0.1% | 5.7% | 90.4% | 9.6% |
| | | element | | | | | | | | | |
| VVT1127 | 0 | SV40Early + | 0.6% | 0.4% | 0.3% | 1.7% | 1.3% | 0.0% | 10.2% | 85.5% | 14.5% |
| | | MAZ element | | | | | | | | | |
| VVT1118 | 1 | bGH | 0.0% | 0.6% | 0.3% | 3.2% | 1.2% | 0.3% | 0.0% | 94.4% | 5.6% |
| VVT1123 | 1 | bGH + | 0.3% | 0.1% | 0.0% | 2.1% | 1.0% | 0.3% | 0.0% | 96.2% | 3.8% |
| | | SV40Late | | | | | | | | | |
| VVT1122 | 4 | bGH | 0.4% | 0.1% | 0.0% | 0.0% | 0.0% | 1.5% | 0.0% | 98.0% | 2.0% |
| VVT1121 | 4 | bGH + | 0.0% | 0.3% | 0.0% | 0.0% | 0.0% | 0.2% | 0.0% | 99.5% | 0.5% |
| | | SV40Late | | | | | | | | | |
| VVT1139 | 6 | bGH | 0.0% | 0.0% | 0.0% | 0.0% | 0.0% | 1.6% | 0.0% | 98.4% | 1.6% |
| VVT1128 | 6 | bGH + | 0.0% | 0.0% | 0.0% | 0.0% | 0.0% | 0.2% | 0.0% | 99.8% | 0.2% |
| | | SV40Late | | | | | | | | | |

Positions with bolded numbers indicate positions where cryptic splice was mutated in transgene template redesigns.
Templates had either 0, 1 (position 3078), 4 (positions 274, 723, 1830, and 3078), or 6 (6, 132, 274, 723, 1830, and 3078) cryptic sites mutated. Data for construct VVT1254 in cynomolgus monkeys is shown here for comparison.
**Calculated using only positions listed in table.

The splicing pattern of ALB-anti-TfR: GAA fusion transcripts was evaluated by short-read RNA sequencing of primary human hepatocytes (PHH) from xenografted mouse livers (Phoenix Bio) following administration of one of constructs VVT1125, VVT1126, or VVT1129 and LNP-g9860. Cryptic splicing at previously identified sites in anti-TfR: GAA (nucleotide positions 1857 and 3015) and also in anti-TfR: GAA redesigns (nucleotide position 2961) were evaluated (Table 65). Mutating cryptic splice sequences eliminated cryptic splicing at those positions. Comparing construct VVT1125 to construct VVT1129, adding SV40Late in tandem with bGH decreased cryptic splicing compared to bGH alone.

TABLE 65
Percentages of Unintended Transcript in ALB-anti-TfR:GAA Fusion Transcripts Following Administration
of AAV Packaged with anti-TfR:GAA Redesigns and LNP-g9860 in Primary Human Hepatocytes.

| | # | | Nucleotide Position in anti-TfR:GAA | | | | |
|---|---|---|---|---|---|---|---|
| | Cryptic | | (Corresponding Position in anti-CD63:GAA) | | | Estimated | Estimated |
| | Sites | PolyA | 1857 | 2961 | 3105 | Intended | Unintended |
| Template | Removed | Sequence | (1830) | (2934) | (3078) | Transcripts** | Transcripts** |
| VVT1126 | 0 | bGH | 1.3% | 0.4% | 19.1% | 79.2% | 20.8% |
| VVT1129 | 2 | bGH | 0.0% | 0.7% | 0.0% | 99.3% | 0.7% |
| VVT1125 | 2 | bGH + | 0.1% | 0.0% | 0.0% | 99.9% | 0.1% |
| | | SV40Late | | | | | |

Positions with bolded numbers indicate positions where cryptic splice was mutated in transgene template redesigns.
Templates had either 0 or 2 (positions 1857 and 3105) cryptic sites mutated.
**Calculated using only positions listed in table.

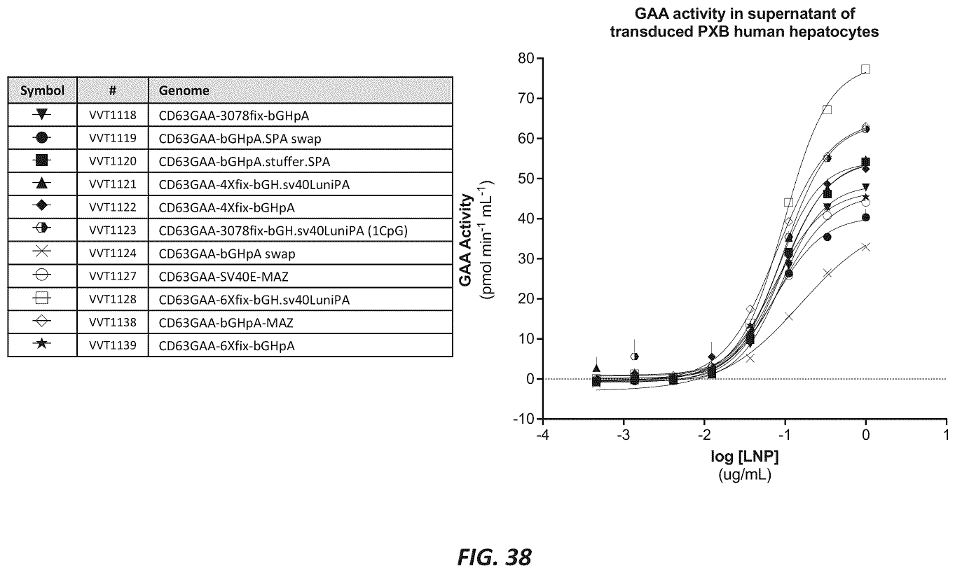

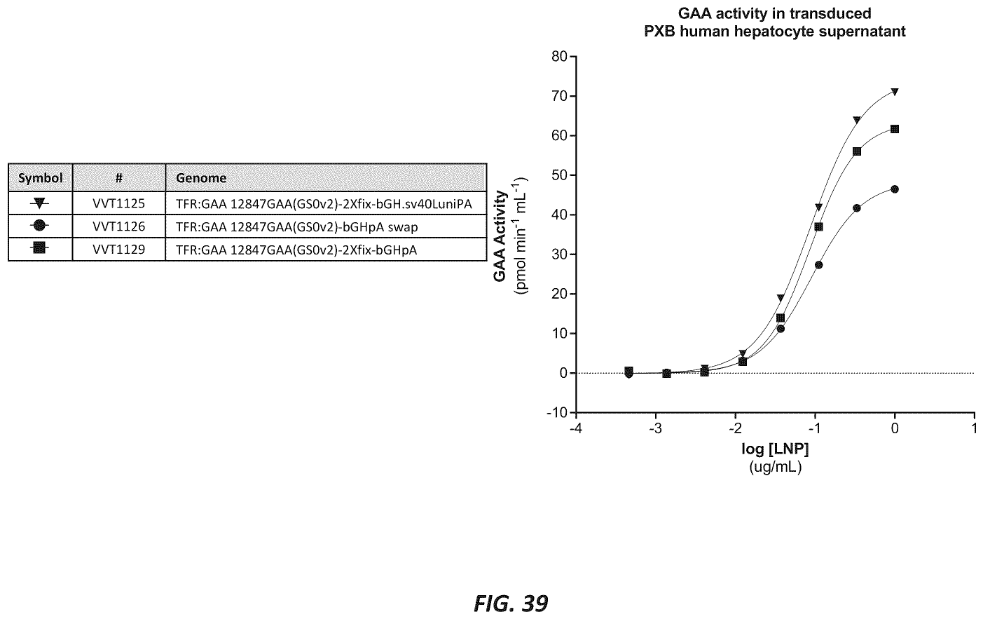

To test the impact of altering cryptic splice sites and polyA sequences on transcript splicing and gene expression following gene insertion, we evaluated gene insertion-mediated expression in human hepatocytes in vitro. Specifically, various sequence-optimized insertion DNA templates for both anti-CD63:GAA or anti-TfR:GAA were first packaged into AAV2. Next, primary human hepatocytes from xenografted mouse livers (Phoenix Bio) were seeded into 96-well plates and treated with AAV2 viruses at a fixed MOI of 6e4, plus variable levels of LNP-g9860. After incubating the cells for 7 days at 37° C., 5% CO₂, supernatants were collected and evaluated for GAA activity using a fluorometric Lysosomal alpha-Glucosidase Activity Assay Kit (Abcam), according to the manufacturer's instructions. We found that removing cryptic splice sites and strengthening polyA-mediated termination of transcription had no negative impact on expression from anti-CD63:GAA (FIG. 38) and anti-TfR:GAA insertion templates (FIG. 39). Engineering anti-CD63:GAA and anti-TfR:GAA insertion templates to remove cryptic splice sites and strengthen polyA sequences resulted in equivalent or enhanced GAA secretion versus unmodified templates.

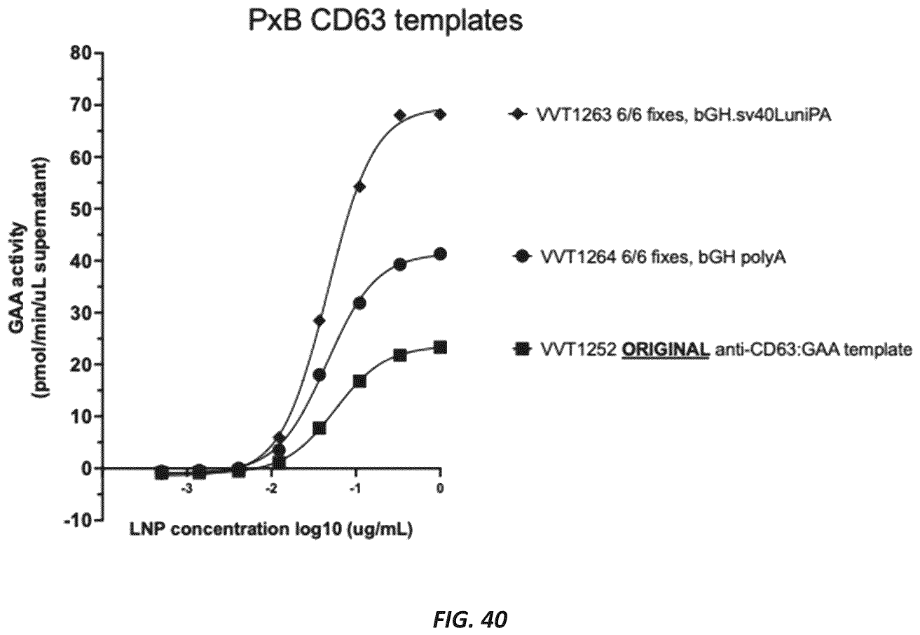

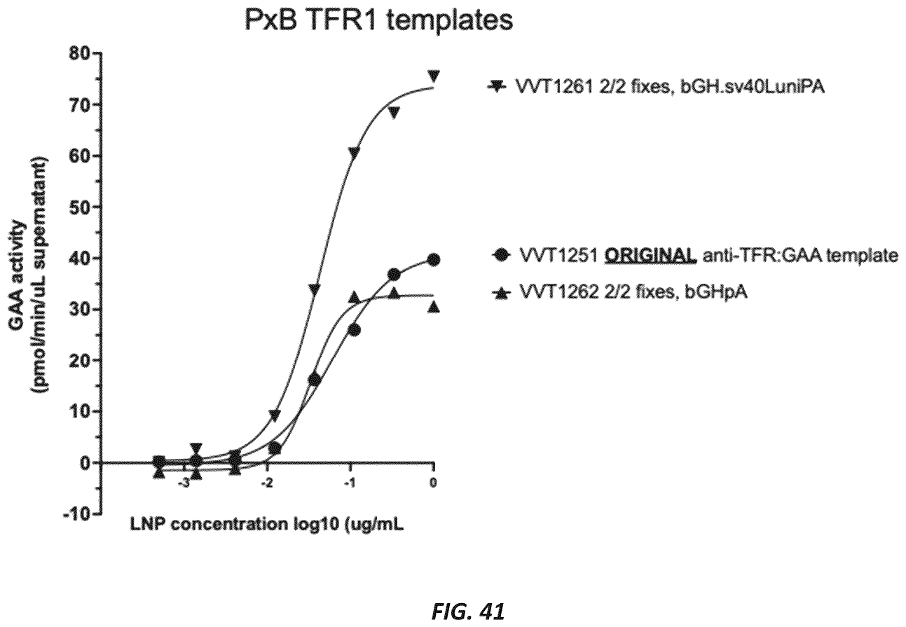

The experiment was repeated using the top cantidates from the first screen in addition to the original codon-optimized anti-CD63:GAA and anti-TFR:GAA insertion templates in which the cryptic splicing was observed. See Table 66. Western blots on lysates is done to assay for loss/gain of translated splice-fusions of GAA. The secreted GAA activity assay was repeated to confirm the results from FIGS. 38-39. The secreted GAA activity assay was performed on supernatants 7 days after gene insertion of AAV template and titrated amounts of LNP. Addition of the unidirectional SV40 late polyA (sv40LuniPA) sequence (VVT1261, VVT1263) improved expression/secretion of GAA compared to the original templates VVT1251 and VVT1262. See FIGS. 40 and 41. Unexpectedly, we observed fold-level increases in the level of GAA activity achievable when combining synonymous substitutions at cryptic 5′ splice sites with a tandem poly(A) sequence comprised of bovine growth hormone poly(A) and this unidirectional variant of SV40 poly(A) in the “late” orientation (collectively referred to as bGH-SV40Luni). Mutation of splice sites showed improvement in GAA expression/secretion in the anti-CD63:GAA template in this experiment. RNA is extracted from the cell lysates, and RNAseq is performed. The decrease in aberrant GAA-albumin splice product transcripts is confirmed by this analysis. Western blots on lysates are probed for GAA and albumin. The new templates show a decrease in band intensity in sizes corresponding to GAA-albumin splice fusions (˜180 kDa), with increased band intensity in the correctly spliced anti-CD63:GAA or anti-TFR:GAA product (˜123 kDa).

TABLE 66
Anti-CD63:GAA and Anti-TfR:GAA Templates for Second PHH Experiment.

| Construct | Description |
|---|---|
| VVT1251 - pINT ITR130 12847scfv GA 0 CpG GAA | Original construct |
| SV40 pA (SEQ ID NO: 763) | |
| VVT1261 - AAV2 pAAV-TfRIGAA(GS0v2)-2Xfix- | 1830 & 3078 cryptic splice sites in GAA corrected & |
| bGH.sv40LuniPA (SEQ ID NO: 764) | original polyA replaced w/tandem bGH + SV40-late |
| | pA w/reverse strand AAUAAAs mutated |
| VVT1262 - AAV2 pAAV-TfRIGAA(GS0v2)-2Xfix- | 1830 & 3078 GAA cryptic splice site corrections & |
| bGHpA (SEQ ID NO: 765 | original SV40-early poly A replaced w/bGHpA |
| VVT1252 - pVPC_CD63GAA130 bp (SEQ ID NO: | Original construct |
| 767) | |
| VVT1263 - AAV2 pAAV-CD63GAA-6Xfix- | 6 cryptic splice site corrections & original SV40-early |
| bGH.sv40LuniPA (SEQ ID NO: 777) | pA replaced w/tandem bGH + SV40-late pA w/ |
| | reverse strand AAUAAAs mutated |
| VVT1264 - AAV2 pAAV-CD63GAA-6Xfix-bGHpA | 6 cryptic splice site corrections & original SV40-early |
| (SEQ ID NO: 778) | pA replaced w/bGHpA |

Recombinant AAV8 viral vectors are designed for in vivo validation in Gaa^{-/-}/Tfrc^{hum} mice or Gaa^{-/-}/Cd63^{hum} mice, and the constructs are validated in the mice. The constructs tested in vivo are shown in Table 67.

TABLE 67
Anti-CD63:GAA and Anti-TfR:GAA Templates for In Vivo Experiment.

| Construct | Description |
|---|---|
| pINT ITR130 anti-TfR1 GA 0 CpG GAA SV40 pA | Original construct |
| (SEQ ID NO: 763) | |
| pVPC.anti-CD63:GAA 130 bp (SEQ ID NO: 767) | Original construct |
| AAV8.anti- | 1830 & 3078 GAA cryptic splice sites corrected |
| TfR1:GAA(GS0v2).2Xfix.bGH.SV40LuniPA (SEQ ID | Original pA replaced w/tandem bGH + SV40-late pA |
| NO: 764) | w/reverse strand AAUAAAs mutated |
| AAV8 pAAV-anti-TfR1:GAA(GS0v2)-bGHpA swap | Original PolyA replaced w/bGHpA |
| (SEQ ID NO: 766) | |
| AAV8.anti-TfR1:GAA(GS0v2).2Xfix.bGHpA (SEQ | 1830 & 3078 GAA cryptic splice sites corrected |
| ID NO: 765) | Original SV40-early pA replaced w/bGHpA |
| AAV8.anti-CD63:GAA.6Xfix.bGH.SV40LuniPA | 6 cryptic splice site corrections & original SV40-early |
| (SEQ ID NO: 777) | pA replaced w/tandem bGH + SV40-late pA w/ |
| | reverse strand AAUAAAs mutated |
| AAV8.anti-CD63:GAA.6Xfix.bGHpA (SEQ ID NO: | 6 cryptic splice site corrections & original SV40-early |
| 778) | pA replaced w/bGHpA |

### Example 15. Anti-TfR:GAA Template Validation in Tfrc^{hum/hum};Gaa^{-/-} Mice

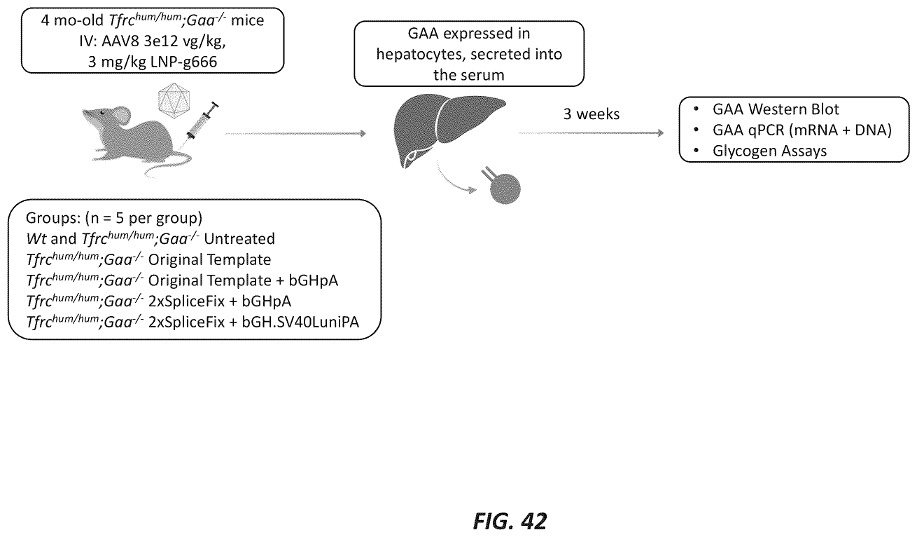

To confirm that the optimized anti-TfR:GAA DNA templates designed to minimize mis-splicing did not affect GAA activity or phenotype rescue in disease model mice, we treated 4-month-old Tfrc^{hum/hum}/Gaa^{-/-} mice with 3 mg/kg LNP-g666 and 3e12 vg/kg AAV8 delivering each of the templates shown in FIG. 42 (AAV8 anti-TfR1 GA OCpG GAA SV40 pA (SEQ ID NO: 763; original template); AAV8 anti-TfR1:GAA(GS0v2)-bGHpA swap (SEQ ID NO: 766; Original template+bGHpA); AAV8.anti-TfR1:GAA(GS0v2).2×fix.bGHpA (SEQ ID NO: 765; 2×SpliceFix+bGHpA); AAV8.anti-TfR1:GAA(GS0v2).2×fix.bGH.SV40LuniPA (SEQ ID NO: 764; 2×SpliceFix+bGH.SV40LuniPA; 5 animals per group). Controls were untreated Tfrc^{hum/hum};Gaa^{-/-} and Tfrc^{hum/hum};Gaa^{+/+} mice. We harvested tissues at 3 weeks post-injection snap frozen in LN2, for GAA western blot and glycogen quantification (methods the same as the previous examples).

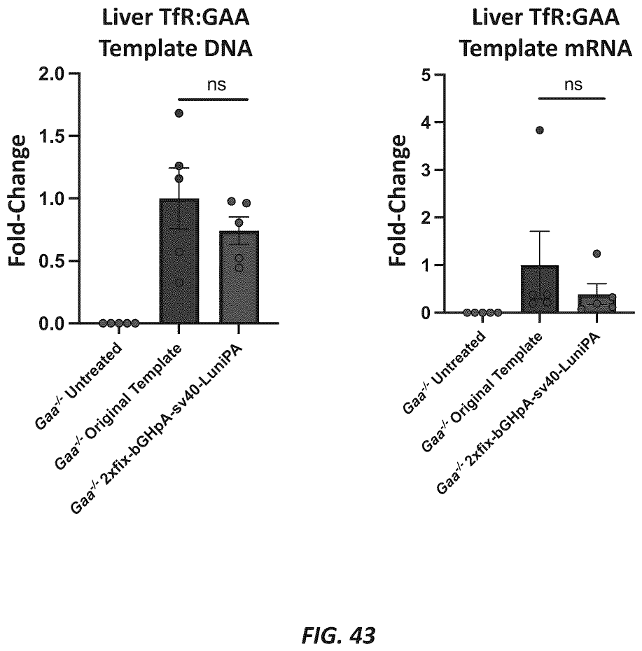

RT-qPCR. Transgene anti-TfR:GAA DNA in liver nucleotide preps was quantified by Tagman using standard protocols. There was no statistically significant difference between the original template and the new edited templates in DNA template delivery to the liver or anti-TfR:GAA mRNA expression. See FIG. 43.

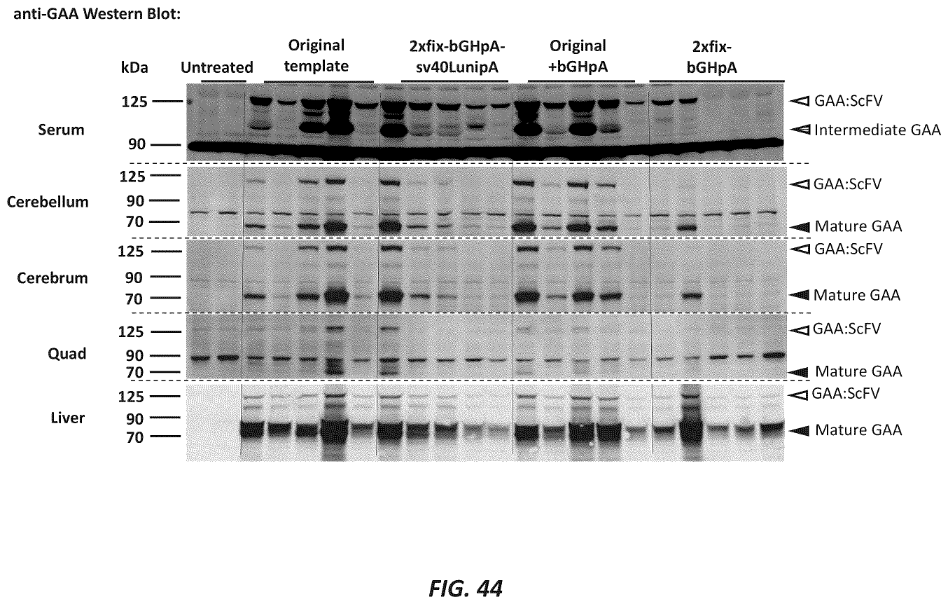

Western blot. Tissue lysates were prepared by lysis in RIPA buffer with protease inhibitors (1861282, Thermo Fisher, Waltham, MA, USA). Tissue lysates were homogenized with a bead homogenizer (FastPrep5, MP Biomedicals, Santa Ana, CA, USA). Cells or tissue lysates were run on SDS-PAGE gels using the Novex system (LifeTech Thermo, XP04200BOX, LC2675, LC3675, LC2676). Gels were transferred to low-fluorescence polyvinylidene fluoridev (PVDF) membrane (IPFL07810, LI-COR, Lincoln, NE, USA) and stained with Revert 700 Total Protein Stain (TPS; 926-11010 LI-COR, Lincoln, NE, USA), followed by blocking with Odyssey blocking buffer (927-500000, LI-COR, Lincoln, NE, USA) in Tris buffer saline with 0.1% Tween 20 and staining with antibodies against GAA (ab137068, Abcam, Cambridge, MA, USA), or anti-GAPDH (ab9484, Abcam, Cambridge, MA, USA) and the appropriate secondary (926-32213 or 925-68070, LI-COR, Lincoln, NE, USA). Blots were imaged with a LI-COR Odyssey CLx. Protein band intensity was quantified in LI-COR Image Studio software. The quantification of the mature 77 kDa GAA band for each sample was determined by normalizing to the lane's TPS signal (loading control). As shown in FIG. 44 (showing expression in serum cerebellum, cerebrum, quad, and liver), the 2×fix-bGHpA-sv40LunipA template was as effective at delivering GAA to the brain as the original lead template. There was some variability in the expression between animals, but overall, the amount of anti-TfR:GAA protein in the serum correlated with the amount of GAA delivered to the brain. There was no statistically significant difference between the original template and the new edited templates in anti-TfR:GAA protein expression. Anti-TfR:GAA delivery to target tissues was also the same across treatment groups.

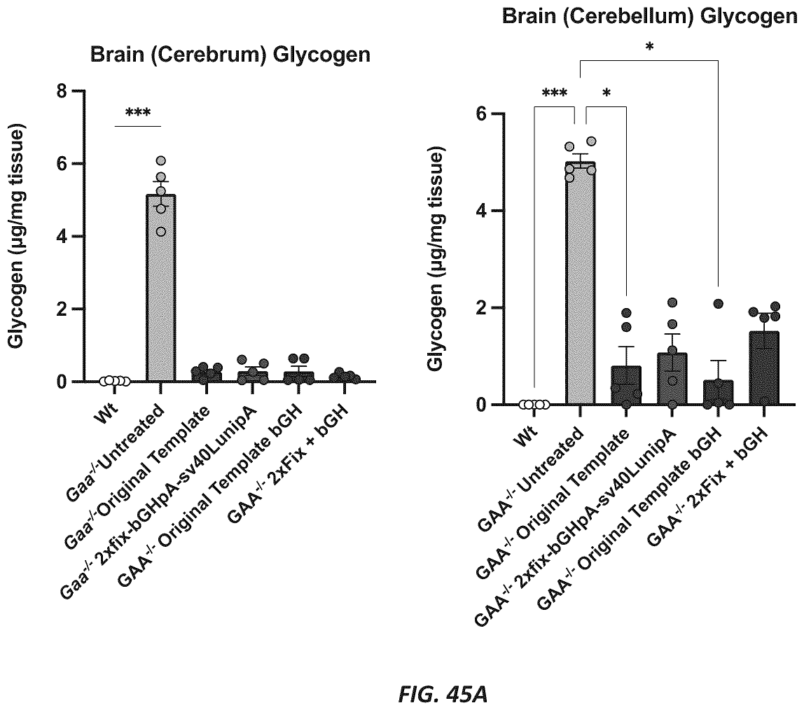

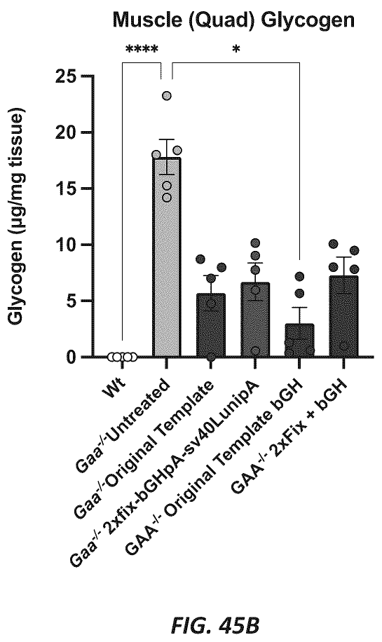

Glycogen quantification. Tissue lysates were prepared by lysis in water with protease inhibitors (1861282, Thermo Fisher, Waltham, MA, USA). Tissue lysates were homogenized with a bead homogenizer (FastPrep5, MP Biomedicals, Santa Ana, CA, USA). Glycogen was quantified using Glycogen Assay Kit (Abam ab65620) as per manufacturer's protocol. As shown in FIG. 45A-45B, the 2×fix-bGHpA-sv40LunipA template was as effective as the original lead template at rescuing the glycogen storage phenotype in the brain (cerebrum, cerebellum) and muscle (quad) of Gaa^{-/-};Tfrc^{hu/hu} mice.

### Example 16. Anti-TfR:GAA Template Validation in Albumin Humanized Mice

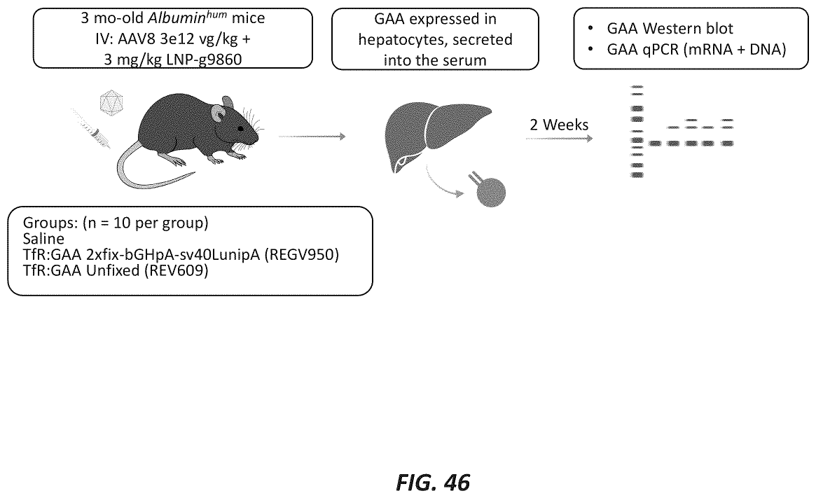

The optimized anti-TfR:GAA DNA templates designed to minimize mis-splicing were tested in humanized albumin mice to assess whether editing two cryptic splice sites in the GAA sequence prevented cryptic splicing to albumin exon 2 and to assess whether a tandem bGHpA+SV40LuniPA increases likelihood of transcript termination. The experimental setup is shown in FIG. 46. Three-month-old Alb^{hum/hum} mice were treated intravenously with 3 mg/kg LNP-g9860 3e12 vg/kg of either REGV609 (original template with cryptic splice sites) or REGV950 (new lead template with 2 fixed splice sites and bGHpA+SV40LuniPA) (i.e., AAV8 anti-TfR1 GA OCpG GAA SV40 pA (SEQ ID NO: 763; original template); AAV8.anti-TfR1:GAA(GS0v2).2×fix.bGH.SV40LuniPA (SEQ ID NO: 764; 2×SpliceFix+bGH.SV40LuniPA)). Controls were saline-injected Alb^{hum/hum} mice. Tissues and serum were harvested and flash-frozen in LN2 at 2 weeks post-injection.

For western blot, tissue lysates were prepared by lysis in RIPA buffer with protease inhibitors (1861282, Thermo Fisher, Waltham, MA, USA). Tissue lysates were homogenized with a bead homogenizer (FastPrep5, MP Biomedicals, Santa Ana, CA, USA). Cells or tissue lysates were run on SDS-PAGE gels using the Novex system (LifeTech Thermo, XP04200BOX, LC2675, LC3675, LC2676). Gels were transferred to low-fluorescence polyvinylidene fluoridev (PVDF) membrane (IPFL07810, LI-COR, Lincoln, NE, USA) and stained with Revert 700 Total Protein Stain (TPS; 926-11010 LI-COR, Lincoln, NE, USA), followed by blocking with Odyssey blocking buffer (927-500000, LI-COR, Lincoln, NE, USA) in Tris buffer saline with 0.1% Tween 20 and staining with antibodies against GAA (ab137068, Abcam, Cambridge, MA, USA), or anti-GAPDH (ab9484, Abcam, Cambridge, MA, USA) and the appropriate secondary (926-32213 or 925-68070, LI-COR, Lincoln, NE, USA). Blots were imaged with a LI-COR Odyssey CLx. Protein band intensity was quantified in LI-COR Image Studio software. The background-corrected intensity of indicated GAA protein bands were quantified using ImageStudio software (Liborio). Individual bands were divided by the Total Protein Stain signal to normalize the signal for protein loading. Fold change per group was calculated compared to the REGV609 group, such that the average of the REGV609 group is equal to 1. For RT-qPCR, transgene anti-TfR:GAA DNA in liver nucleotide preps was quantified by Taqman using standard protocols.

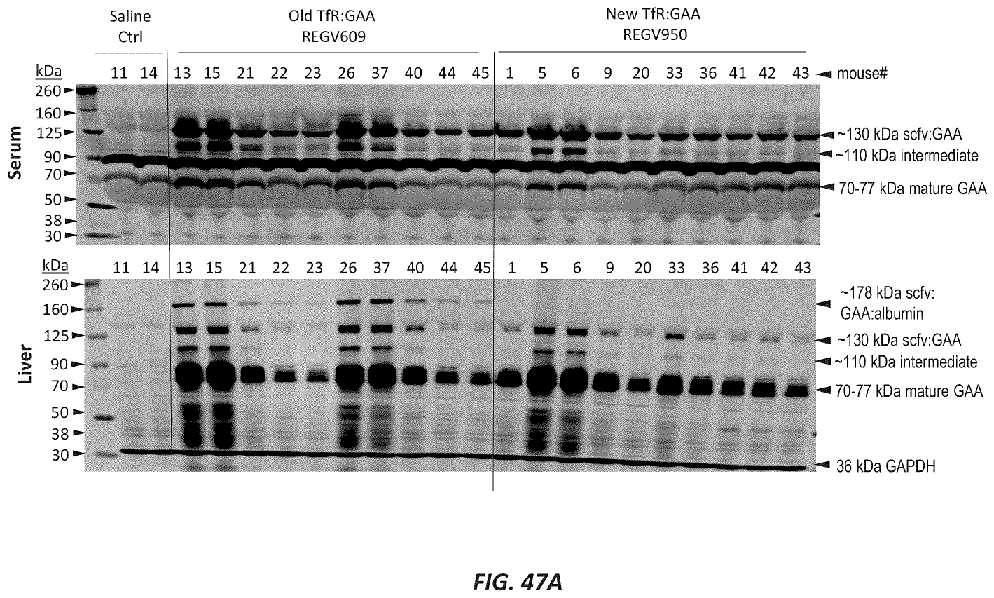

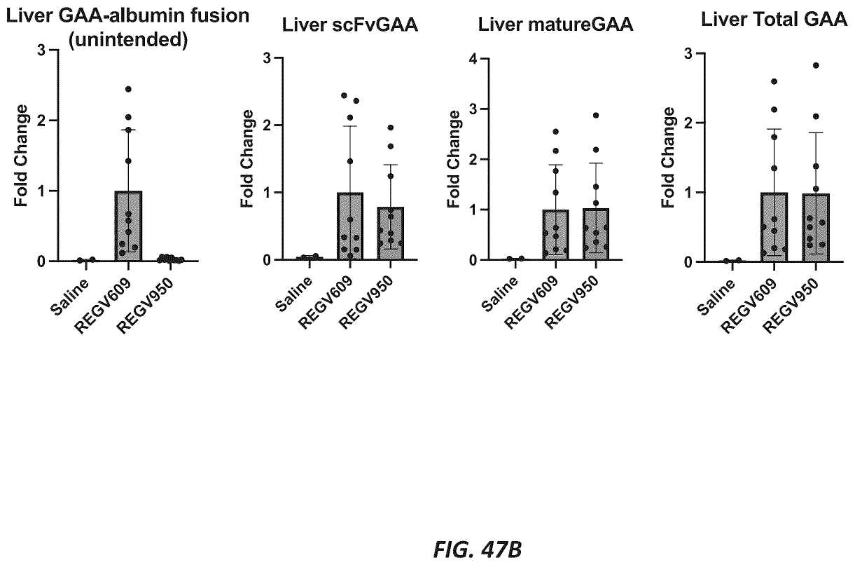

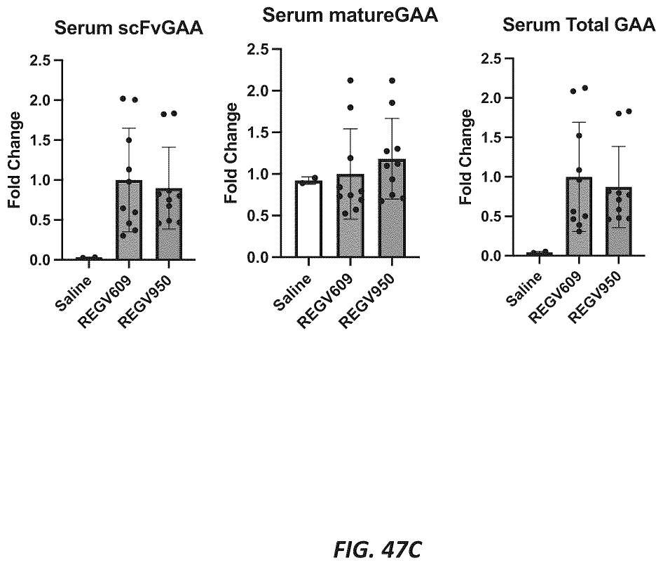

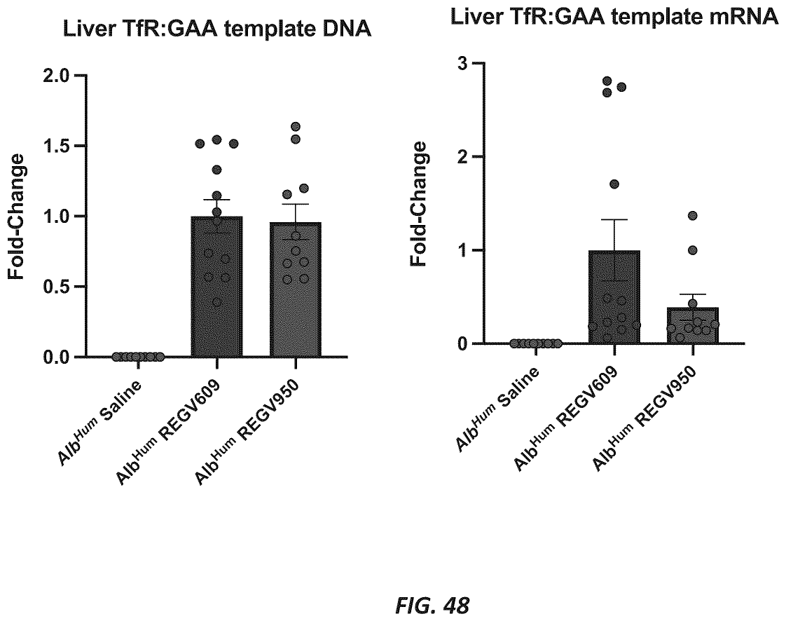

The new lead REGV950 template did not express the unintended anti-TfR:GAA-albumin fusion protein in the liver, as seen by an absence of the 178 kDa band on the western blot, while the original REGV609 template did express this unintended product. See FIG. 47A. All other processed forms of GAA protein were unchanged between the two treatment groups. See FIGS. 47B-47C. These results are consistent with the previous RNAseq findings both in vitro and in vivo. There was no statistically significant difference between the original template and the new edited templates in DNA template delivery to the liver or anti-TfR:GAA mRNA expression. See FIG. 48.

1. A composition comprising a nucleic acid construct comprising a coding sequence for a multidomain therapeutic protein comprising a delivery domain fused to a lysosomal alpha-glucosidase polypeptide, wherein the lysosomal alpha-glucosidase coding sequence is CpG-depleted relative to a wild type lysosomal alpha-glucosidase coding sequence, wherein the delivery domain is a TfR-binding delivery domain.

2.-80. (canceled)

81. The composition of claim 1 in combination with a nuclease agent that targets a nuclease target site in a target genomic locus.

82.-108. (canceled)

109. A cell comprising the composition of claim 1.

110.-113. (canceled)

114. A method of inserting a nucleic acid encoding a multidomain therapeutic protein comprising a delivery domain fused to a lysosomal alpha-glucosidase into a target genomic locus in a cell or a population of cells, comprising administering to the cell or the population of cells the composition of claim 81,
wherein the nuclease agent cleaves the nuclease target site in the target genomic locus, and the nucleic acid construct or the nucleic acid encoding the multidomain therapeutic protein is inserted into the target genomic locus.

115. (canceled)

116. A method of expressing a multidomain therapeutic protein comprising a delivery domain fused to a lysosomal alpha-glucosidase in a cell or a population of cells, comprising administering to the cell or the population of cells the composition of claim 1,
wherein the coding sequence for the multidomain therapeutic protein is operably linked to a promoter in the nucleic acid construct and is expressed in the cell or population of cells.

117. A method of expressing a multidomain therapeutic protein comprising a delivery domain fused to a lysosomal alpha-glucosidase from a target genomic locus in a cell or a population of cells, comprising administering to the cell or the population of cells the composition of claim 81,
wherein the nuclease agent cleaves the nuclease target site in the target genomic locus, the nucleic acid construct or the coding sequence for the multidomain therapeutic protein is inserted into the target genomic locus to create a modified target genomic locus, and the multidomain therapeutic protein comprising the delivery domain fused to the lysosomal alpha-glucosidase is expressed from the modified target genomic locus.

118.-125. (canceled)

126. A method of inserting a nucleic acid encoding a multidomain therapeutic protein comprising a delivery domain fused to a lysosomal alpha-glucosidase into a target genomic locus in a cell in a subject, comprising administering to the subject the composition of any one of claims 81-108 claim 81,
wherein the nuclease agent cleaves the nuclease target site in the target genomic locus, and the nucleic acid construct or the nucleic acid encoding the multidomain therapeutic protein is inserted into the target genomic locus.

127. (canceled)

128. A method of expressing a multidomain therapeutic protein comprising a delivery domain fused to a lysosomal alpha-glucosidase protein in a cell in a subject, comprising administering to the subject the composition of claim 1,
wherein the coding sequence for the multidomain therapeutic protein is operably linked to a promoter in the nucleic acid construct and is expressed in the cell.

129. A method of expressing a multidomain therapeutic protein comprising a delivery domain fused to a lysosomal alpha-glucosidase protein from a target genomic locus in a cell in a subject, comprising administering to the subject the composition of claim 81,
wherein the nuclease agent cleaves the nuclease target site in the target genomic locus, the nucleic acid construct or the coding sequence for the multidomain therapeutic protein is inserted into the target genomic locus to create a modified target genomic locus, and the multidomain therapeutic protein comprising the delivery domain fused to the lysosomal alpha-glucosidase is expressed from the modified target genomic locus.

130.-136. (canceled)

137. A method of treating a lysosomal alpha-glucosidase deficiency in a subject in need thereof, comprising administering to the subject the composition of claim 1,
wherein the coding sequence for the multidomain therapeutic protein is operably linked to a promoter in the nucleic acid construct and is expressed in the subject.

138. A method of treating a lysosomal alpha-glucosidase deficiency in a subject in need thereof, comprising administering to the subject the composition of claim 81,
wherein the nuclease agent cleaves the nuclease target site in the target genomic locus, the nucleic acid construct or the coding sequence for the multidomain therapeutic protein is inserted into the target genomic locus to create a modified target genomic locus, and the multidomain therapeutic protein comprising the delivery domain fused to the lysosomal alpha-glucosidase is expressed from the modified target genomic locus.

139. (canceled)

140. A method of reducing glycogen accumulation in a tissue in a subject in need thereof, comprising administering to the subject the composition of claim 1,
wherein the coding sequence for the multidomain therapeutic protein is operably linked to a promoter in the nucleic acid construct and is expressed in the subject and reduces glycogen accumulation in the tissue.

141. A method of reducing glycogen accumulation in a tissue in a subject in need thereof, comprising administering to the subject the composition of claim 81,
wherein the nuclease agent cleaves the nuclease target site in the target genomic locus, the nucleic acid construct or the coding sequence for the multidomain therapeutic protein is inserted into the target genomic locus to create a modified target genomic locus, and the multidomain therapeutic protein comprising the delivery domain fused to the lysosomal alpha-glucosidase is expressed from the modified target genomic locus and reduces glycogen accumulation in the tissue.

142. (canceled)

143. (canceled)

144. A method of treating Pompe disease in a subject in need thereof, comprising administering to the subject the composition of claim 1,
wherein the coding sequence for the multidomain therapeutic protein is operably linked to a promoter in the nucleic acid construct and is expressed in the subject, thereby treating the Pompe disease.

145. A method of treating Pompe disease in a subject in need thereof, comprising administering to the subject the composition of claim 81,
wherein the nuclease agent cleaves the nuclease target site in the target genomic locus, the nucleic acid construct or the coding sequence for the multidomain therapeutic protein is inserted into the target genomic locus to create a modified target genomic locus, and the multidomain therapeutic protein comprising the delivery domain fused to the lysosomal alpha-glucosidase is expressed from the modified target genomic locus, thereby treating the Pompe disease.

146.-155. (canceled)

156. A method of preventing or reducing the onset of a sign or symptom of Pompe disease in a subject in need thereof, comprising administering to the subject the composition of claim 1,
wherein the coding sequence for the multidomain therapeutic protein is operably linked to a promoter in the nucleic acid construct and is expressed in the subject, thereby preventing or reducing the onset of a sign or symptom of the Pompe disease in the subject.

157. A method of preventing or reducing the onset of a sign or symptom of Pompe disease in a subject in need thereof, comprising administering to the subject the composition of claim 81,
wherein the nuclease agent cleaves the nuclease target site, the nucleic acid construct or the coding sequence for the multidomain therapeutic protein is inserted into the target genomic locus to create a modified target genomic locus, and the multidomain therapeutic protein comprising the delivery domain fused to the lysosomal alpha-glucosidase is expressed from the modified target genomic locus, thereby preventing or reducing the onset of a sign or symptom of the Pompe disease in the subject.

158.-188. (canceled)

189. A composition comprising a nucleic acid construct comprising a coding sequence for a multidomain therapeutic protein comprising a delivery domain fused to a lysosomal alpha-glucosidase polypeptide, wherein the lysosomal alpha-glucosidase coding sequence is CpG-depleted relative to a wild type lysosomal alpha-glucosidase coding sequence, wherein the delivery domain is a CD63-binding delivery domain.

190. The composition of claim 189 in combination with a nuclease agent that targets a nuclease target site in a target genomic locus.

191. A cell comprising the composition of claim 189.

192. A method of inserting a nucleic acid encoding a multidomain therapeutic protein comprising a delivery domain fused to a lysosomal alpha-glucosidase into a target genomic locus in a cell or a population of cells, comprising administering to the cell or the population of cells the composition of claim 190,
wherein the nuclease agent cleaves the nuclease target site in the target genomic locus, and the nucleic acid construct or the nucleic acid encoding the multidomain therapeutic protein is inserted into the target genomic locus.

193. A method of expressing a multidomain therapeutic protein comprising a delivery domain fused to a lysosomal alpha-glucosidase in a cell or a population of cells, comprising administering to the cell or the population of cells the composition of claim 189,
wherein the coding sequence for the multidomain therapeutic protein is operably linked to a promoter in the nucleic acid construct and is expressed in the cell or population of cells.

194. A method of expressing a multidomain therapeutic protein comprising a delivery domain fused to a lysosomal alpha-glucosidase from a target genomic locus in a cell or a population of cells, comprising administering to the cell or the population of cells the composition of claim 190,
wherein the nuclease agent cleaves the nuclease target site in the target genomic locus, the nucleic acid construct or the coding sequence for the multidomain therapeutic protein is inserted into the target genomic locus to create a modified target genomic locus, and the multidomain therapeutic protein comprising the delivery domain fused to the lysosomal alpha-glucosidase is expressed from the modified target genomic locus.

195. A method of inserting a nucleic acid encoding a multidomain therapeutic protein comprising a delivery domain fused to a lysosomal alpha-glucosidase into a target genomic locus in a cell in a subject, comprising administering to the subject the composition of claim 190,
wherein the nuclease agent cleaves the nuclease target site in the target genomic locus, and the nucleic acid construct or the nucleic acid encoding the multidomain therapeutic protein is inserted into the target genomic locus.

196. A method of expressing a multidomain therapeutic protein comprising a delivery domain fused to a lysosomal alpha-glucosidase protein in a cell in a subject, comprising administering to the subject the composition of claim 189,
wherein the coding sequence for the multidomain therapeutic protein is operably linked to a promoter in the nucleic acid construct and is expressed in the cell.

197. A method of expressing a multidomain therapeutic protein comprising a delivery domain fused to a lysosomal alpha-glucosidase protein from a target genomic locus in a cell in a subject, comprising administering to the subject the composition of claim 190,
wherein the nuclease agent cleaves the nuclease target site in the target genomic locus, the nucleic acid construct or the coding sequence for the multidomain therapeutic protein is inserted into the target genomic locus to create a modified target genomic locus, and the multidomain therapeutic protein comprising the delivery domain fused to the lysosomal alpha-glucosidase is expressed from the modified target genomic locus.

198. A method of treating a lysosomal alpha-glucosidase deficiency in a subject in need thereof, comprising administering to the subject the composition of claim 189,
wherein the coding sequence for the multidomain therapeutic protein is operably linked to a promoter in the nucleic acid construct and is expressed in the subject.

199. A method of treating a lysosomal alpha-glucosidase deficiency in a subject in need thereof, comprising administering to the subject the composition of claim 190,
wherein the nuclease agent cleaves the nuclease target site in the target genomic locus, the nucleic acid construct or the coding sequence for the multidomain therapeutic protein is inserted into the target genomic locus to create a modified target genomic locus, and the multidomain therapeutic protein comprising the delivery domain fused to the lysosomal alpha-glucosidase is expressed from the modified target genomic locus.

200. A method of reducing glycogen accumulation in a tissue in a subject in need thereof, comprising administering to the subject the composition of claim 189,
wherein the coding sequence for the multidomain therapeutic protein is operably linked to a promoter in the nucleic acid construct and is expressed in the subject and reduces glycogen accumulation in the tissue.

201. A method of reducing glycogen accumulation in a tissue in a subject in need thereof, comprising administering to the subject the composition of claim 190,
wherein the nuclease agent cleaves the nuclease target site in the target genomic locus, the nucleic acid construct or the coding sequence for the multidomain therapeutic protein is inserted into the target genomic locus to create a modified target genomic locus, and the multidomain therapeutic protein comprising the delivery domain fused to the lysosomal alpha-glucosidase is expressed from the modified target genomic locus and reduces glycogen accumulation in the tissue.

202. A method of treating Pompe disease in a subject in need thereof, comprising administering to the subject the composition of claim 189,
wherein the coding sequence for the multidomain therapeutic protein is operably linked to a promoter in the nucleic acid construct and is expressed in the subject, thereby treating the Pompe disease.

203. A method of treating Pompe disease in a subject in need thereof, comprising administering to the subject the composition of claim 190,
wherein the nuclease agent cleaves the nuclease target site in the target genomic locus, the nucleic acid construct or the coding sequence for the multidomain therapeutic protein is inserted into the target genomic locus to create a modified target genomic locus, and the multidomain therapeutic protein comprising the delivery domain fused to the lysosomal alpha-glucosidase is expressed from the modified target genomic locus, thereby treating the Pompe disease.

204. A method of preventing or reducing the onset of a sign or symptom of Pompe disease in a subject in need thereof, comprising administering to the subject the composition of claim 189,
wherein the coding sequence for the multidomain therapeutic protein is operably linked to a promoter in the nucleic acid construct and is expressed in the subject, thereby preventing or reducing the onset of a sign or symptom of the Pompe disease in the subject.

205. A method of preventing or reducing the onset of a sign or symptom of Pompe disease in a subject in need thereof, comprising administering to the subject the composition of claim 190,
wherein the nuclease agent cleaves the nuclease target site, the nucleic acid construct or the coding sequence for the multidomain therapeutic protein is inserted into the target genomic locus to create a modified target genomic locus, and the multidomain therapeutic protein comprising the delivery domain fused to the lysosomal alpha-glucosidase is expressed from the modified target genomic locus, thereby preventing or reducing the onset of a sign or symptom of the Pompe disease in the subject.
